(19) United States
(12) Patent Application Publication
(10) Pub. No.: US 2025/0295695 A1
(43) Pub. Date: Sep. 25, 2025

(54) COMPOSITIONS AND METHODS FOR MEDIATING EPITOPE ENGINEERING

(71) Applicant: VOR BIOPHARMA INC., Cambridge, MA (US)

(72) Inventors: Abhinav Dhall, Cambridge, MA (US); John Lydeard, Cambridge, MA (US); Tirtha Chakraborty, Cambridge, MA (US)

(73) Assignee: VOR BIOPHARMA INC., Cambridge, MA (US)

(21) Appl. No.: 18/853,868

(22) PCT Filed: Apr. 4, 2023
(86) PCT No.: PCT/US2023/065333

§ 371 Date: Oct. 3, 2024

Related U.S. Application Data
(60) Provisional application No. 63/327,266, filed on Apr. 4, 2022.
(60) Provisional application No. 63/424,085, filed on Nov. 9, 2022.

(51) Int. Cl. A61K 35/17; A61K 40/11; A61K 40/42; C07K 14/705; C07K 14/715; C07K 14/725; C07K 16/28; C12N 5/0789; C12N 9/22; C12N 15/11; C12N 15/90

(52) U.S. Cl. CPC A61K 35/17; A61K 40/11; A61K 40/4211; A61K 40/4217; A61K 40/4222; A61K 40/4224; C07K 14/7051; C07K 14/70517; C07K 14/70521; C07K 14/70596; C07K 14/7155; C07K 16/2866; C07K 16/2896; C12N 5/0647; C12N 9/226; C12N 15/111; C12N 15/907; A61K 2239/13; A61K 2239/17; A61K 2239/21; A61K 2239/22; A61K 2239/48; C07K 2317/24; C07K 2317/622; C12N 2310/20; C12N 2510/00

(57) ABSTRACT

Provided herein are compositions and methods for genetically engineering a cell (e.g., a hematopoietic cell) to modify a gene encoding a lineage-specific cell-surface antigen to modify an epitope of the lineage-specific cell-surface antigen recognized by an agent. Also provided are methods involving administering such genetically engineered cells to a subject, such as a subject having a hematopoietic malignancy, as well as the genetically engineered cells themselves.

### RELATED APPLICATIONS

The application claims the benefit under 35 U.S.C. 119 (e) of U.S. Provisional Application No. 63/327,266 filed on Apr. 4, 2022, and U.S. Provisional Application No. 63/424,085 filed on Nov. 9, 2022, each of which is incorporated by reference in its entirety.

### REFERENCE TO AN ELECTRONIC SEQUENCE LISTING

The contents of the electronic sequence listing (V029170014WO00-SEQ-CEW.xml; Size: 570,220 bytes; and Date of Creation: Apr. 3, 2023) is herein incorporated by reference in its entirety.

### BACKGROUND

When a subject is administered an immunotherapy targeting an antigen associated with a disease or condition, e.g., an anti-cancer CAR-T therapy, the therapy can deplete not only the pathological cells intended to be targeted, but also non-pathological cells that may express the targeted antigen. This “on-target, off-disease” effect has been reported for some CAR-T therapeutics, e.g., those targeting CD19 or CD33. If the targeted antigen is expressed on the surface of cells required for survival of the subject, or on the surface of cells the depletion of which is of significant detriment to the health of the subject, the subject may not be able to receive the immunotherapy, or may have to face severe side effects once administered such a therapy.

### SUMMARY

Aspects of the present disclosure describe compositions, methods, strategies, and treatment modalities that address the detrimental on-target, off-disease effects of certain immunotherapeutic approaches, e.g., of immunotherapeutics comprising lymphocyte effector cells targeting a specific antigen in a subject in need thereof, such as CAR-T cells or CAR-NK cells. Some aspects of this disclosure provide compositions, methods, strategies, and treatment modalities related to modifying an epitope of a lineage-specific cell-surface antigen on a hematopoietic cell such that binding of an agent that specifically binds said lineage-specific cell-surface antigen is decreased or eliminated. In some embodiments, the modification of the epitope does not alter (e.g., impair) the function of the lineage-specific cell-surface antigen. In some embodiments, hematopoietic cells comprising an epitope-modified lineage-specific cell-surface antigen are provided that are characterized by decreased or eliminated binding by the agent (e.g., an immunotherapeutic agent such as a CAR-T cells or CAR-NK cells) to the modified epitope. In some embodiments, administration of such a hematopoietic cell comprising an epitope-modified lineage-specific cell-surface antigen, e.g., in combination with the agent, can decrease or mitigate detrimental on-target, off-disease effects in a subject. Some aspects of this disclosure provide compositions and methods for genetic modification (or gene editing) of cells using homology-directed repair (HDR). In some embodiments, methods and compositions described herein combine sequence-specificity (e.g., of a CRISPR/Cas system) with HDR-mediated gene editing, enabling targeted integration of sequences from a template polynucleotide at a target sequence specified by homology of portions of a template polynucleotide to the target sequence. In some embodiments, methods and compositions utilizing HDR described herein are characterized by a high editing efficiency and a high rate of survival and/or high viability in the resulting edited cell populations, e.g., in populations of edited human hematopoietic cells, such as, for example, human hematopoietic stem cells. Some aspects of this disclosure provide the benefits of utilizing high efficiency HDR editing to achieve targeted epitope editing and produce modified lineage-specific cell-surface antigens, e.g., that retain functionality, but exhibit reduced or eliminated binding to immunotherapeutic agents targeting the antigen.

Accordingly, some aspects of the present disclosure provides a genetically engineered hematopoietic cell, or descendant thereof, comprising a genomic modification in a gene encoding a lineage-specific cell-surface antigen, wherein the genomic modification alters the amino acid sequence of an epitope that is recognized by an agent that specifically binds the lineage-specific cell-surface antigen resulting in a modified lineage-specific cell-surface antigen, and wherein the modified lineage-specific cell-surface antigen is characterized by reduced binding or no binding of the agent.

In some embodiments, the genomic modification alters 1, 2, 3, 4, or 5 amino acid residues of the lineage-specific cell-surface antigen. In some embodiments, the genomic modification alters no more than 1, no more than 2, no more than 3, no more than 4, or no more than 5 amino acid residues of the lineage-specific cell-surface antigen. In some embodiments, the genomic modification results in a deletion, a substitution, an insertion, or an inversion of one or more amino acid residues, or a combination thereof. In some embodiments, the genomic modification results in a substitution of one or more amino acid residues.

In some embodiments, the epitope is characterized by an endogenous post-translational modification. In some embodiments, the endogenous post-translation modification is a glycosylation.

In some embodiments, the agent is an immunotherapeutic agent. In some embodiments, the immunotherapeutic agent comprises an antibody or an antigen-binding fragment thereof. In some embodiments, the modified lineage-specific cell-surface antigen is not recognized by the agent. In some embodiments, the modified lineage-specific cell-surface antigen is recognized by a second agent that specifically binds to a different region of the lineage-specific cell-surface antigen than the epitope recognized by the first agent.

In some embodiments, the genomic modification does not substantially alter the function of the lineage-specific cell-surface antigen. In some embodiments, the genomic modification does not substantially alter the expression of the lineage-specific cell-surface antigen. In some embodiments, the genomic modification does not substantially alter the viability or growth of the cell. In some embodiments, the hematopoietic cell, or descendant thereof retains the capacity to differentiate normally compared to a reference population of hematopoietic cells, optionally a population of hematopoietic cells not comprising the genomic modification.

In some embodiments, the hematopoietic cell is a hematopoietic stem cell (HSC). In some embodiments, the hematopoietic cell is a CD34+ cell. In some embodiments, the hematopoietic cell is obtained from bone marrow, blood, umbilical cord, or peripheral blood mononuclear cells (PBMCs). In some embodiments, the hematopoietic cell is human.

In some embodiments, the lineage-specific cell-surface antigen is selected from the group consisting of CD123, CD47, CD34, CD38, CD19, CD33, CLL-1, CD30, CD5, CD6, CD7, EMR2, and BCMA. In some embodiments, the lineage-specific cell-surface antigen is CD123. In some embodiments, the lineage-specific cell-surface antigen is CD38. In some embodiments, the lineage-specific cell-surface antigen is CD19. In some embodiments, the lineage-specific cell-surface antigen is EMR2. In some embodiments, the lineage-specific cell-surface antigen is CD5. In some embodiments, the lineage-specific cell-surface antigen is CD47. In some embodiments, the lineage-specific cell-surface antigen is CD34.

In some embodiments, the epitope is encoded by exon 3 and/or exon 4 of the gene encoding CD123. In some embodiments, the epitope is a region of CD123 bound by murine anti-CD123 antibody 7G3, a humanized variant thereof (e.g., antibody CSL-362), or talacotuzumab. In some embodiments, the agent comprises murine anti-CD123 antibody 7G3, a humanized variant thereof (e.g., antibody CSL-362), or talacotuzumab. In some embodiments, the epitope comprises 1, 2, 3, 4, or 5 of the amino acids at positions 51, 59, 61, 82, or 84 of a wildtype gene encoding CD123. In some embodiments, the genomic modification results in a deletion, a substitution, an insertion, or an inversion of one or more of the amino acids at positions 51, 59, 61, 82, or 84 of a wildtype gene encoding CD123 or at corresponding positions in a homologous CD123 gene. In some embodiments, the genomic modification results in a substitution of one or more (e.g., 1, 2, 3, 4, or all) of the amino acids at positions 51, 59, 61, 82, or 84 of a wildtype gene encoding CD123 or at corresponding positions in a homologous CD123 gene. In some embodiments, the one or more substitutions are conservative substitutions. In some embodiments, the genomic modification results in a substitution of the amino acid at position 51 of a wildtype gene encoding CD123 or at a corresponding position in a homologous CD123 gene. In some embodiments, the genomic modification results in a substitution of a lysine for glutamic acid at position 51 of a wildtype gene encoding CD123 or at a corresponding position in a homologous CD123 gene.

In some embodiments, the epitope is encoded by exon 7 of the gene encoding CD38. In some embodiments, the epitope is a region of CD38 bound by murine anti-CD38 antibody HB7, a humanized variant thereof, or daratumumab. In some embodiments, the agent comprises murine anti-CD38 antibody HB7, a humanized variant thereof, or daratumumab. In some embodiments, the epitope comprises 1, 2, 3, 4, or 5 of the amino acids at positions 270-274 of a wildtype gene encoding CD38. In some embodiments, the genomic modification results in a deletion, a substitution, an insertion, or an inversion of one or more of the amino acids at positions 270-274 of a wildtype gene encoding CD38 or at corresponding positions in a homologous CD38 gene. In some embodiments, the genomic modification results in a substitution of one or more (e.g., 1, 2, 3, 4, or all) of the amino acids at positions 270-274 of a wildtype gene encoding CD38 or at corresponding positions in a homologous CD38 gene. In some embodiments, the one or more substitutions are conservative substitutions. In some embodiments, the genomic modification results in a substitution of the amino acid at position 272 of a wildtype gene encoding CD38 or at a corresponding position in a homologous CD38 gene. In some embodiments, the genomic modification results in a substitution of an arginine, histidine, or alanine for glutamine at position 272 of a wildtype gene encoding CD38 or at a corresponding position in a homologous CD38 gene.

In some embodiments, the epitope is encoded by exon 2 or exon 4 of CD19. In some embodiments, the epitope is a region of CD19 bound by anti-CD19 antibody B43, anti-CD19 antibody FMC63, or a fragment thereof. In some embodiments, the agent comprises anti-CD19 antibody B43, anti-CD19 antibody FMC63, tafasitamab, loncastuximab, blinatumomab, or fragments thereof. In some embodiments, the epitope comprises 1, 2, 3, 4, 5, 6, 7, 8, 9, 10, 11, 12, 13, 14, 15, 16, 17, 18, 19, or 20 of the amino acids at positions 216-224 or 218-238 of a wildtype gene encoding CD19. In some embodiments, the genomic modification results in a deletion, a substitution, an insertion, or an inversion of one or more of the amino acids at positions 163, 164, 216-224 or 218-238 of a wildtype gene encoding CD19 or at corresponding positions in a homologous CD19 gene. In some embodiments, the genomic modification results in a substitution of one or more (e.g., 1, 2, 3, 4, 5, 6, 7, 8, 9, 10, 11, 12, 13, 14, 15, 16, 17, 18, 19, 20, or more, e.g., all) of the amino acids at positions 163, 164, 216-224 or 218-238 of a wildtype gene encoding CD19 or at corresponding positions in a homologous CD19 gene. In some embodiments, the one or more substitutions are conservative substitutions. In some embodiments, the genomic modification results in a substitution of the amino acid at position 163 of a wildtype gene encoding CD19 or at a corresponding position in a homologous CD19 gene. In some embodiments, the genomic modification results in a substitution of a cysteine or a leucine at the amino acid at position 163 of a wildtype gene encoding CD19 or at a corresponding position in a homologous CD19 gene. In some embodiments, the genomic modification results in a substitution of the amino acid at position 163 and 220 of a wildtype gene encoding CD19 or at a corresponding position in a homologous CD19 gene. In some embodiments, the genomic modification results in a substitution of the amino acid at position 163 and 164 of a wildtype gene encoding CD19 or at a corresponding position in a homologous CD19 gene. In some embodiments, the genomic modification results in a substitution of the amino acid at position 163 and 164 of a wildtype gene encoding CD19 or at a corresponding position in a homologous CD19 gene, wherein the substitution of the amino acid at position 163 is a cysteine or a leucine and the substitution of the amino acid at position 164 is a phenylalanine. In some embodiments, the genomic modification results in a substitution of a phenylalanine at the amino acid at position 164 of a wildtype gene encoding CD19 or at a corresponding position in a homologous CD19 gene.

In some embodiments, the epitope comprises 1, 2, 3, 4, 5, or 6 of the amino acids at positions 124, 132, 146, 292, 294, 295, 296, 298, 299, 303, 304, 305, 306, 307, 308, 312, 318, 320, 328, 329, 331, 332, 335, 340, 347, 527, or 708 of a wildtype gene encoding EMR2. In some embodiments, the genomic modification results in a deletion, a substitution, an insertion, or an inversion of one or more of the amino acids at positions 124, 132, 146, 292, 294, 295, 296, 298, 299, 303, 304, 305, 306, 307, 308, 312, 318, 328, 329, 331, 332, 335, 340, 347, 527, or 708 of a wildtype gene encoding EMR2 or at corresponding positions in a homologous EMR2 gene.

In some embodiments, the epitope is a region of CD47 bound by anti-CD47 antibody B6H12, anti-CD47 antibody 2D3, or fragments thereof. In some embodiments, the agent comprises anti-CD47 antibody B6H12, anti-CD47 antibody 2D3, Ligufalimab, or fragments thereof. In some embodiments, the epitope comprises 1, 2, 3, 4, 5, or 6 of the amino acids at positions 117-122 of a wildtype gene encoding CD47. In some embodiments, the epitope comprises 1, 2, 3, or 4 of the amino acids at positions 47, 49, 52-55 or 117-122 of a wildtype gene encoding CD47. In some embodiments, the genomic modification results in a deletion, a substitution, an insertion, or an inversion of one or more of the amino acids at positions 31, 47, 49, 52-55, 117-122, or 124 of a wildtype gene encoding CD47 or at corresponding positions in a homologous CD47 gene. In some embodiments, the one or more substitutions are conservative substitutions. In some embodiments, the genomic modification results in a substitution of one or more of the amino acids at positions 31, 47, 49, 52-55 117-122, or 124 of a wildtype gene encoding CD47 or at a corresponding position in a homologous CD47 gene. In some embodiments, the genomic modification results in a substitution of the amino acid at position 49 of a wildtype gene encoding CD47 or at a corresponding position in a homologous CD47 gene. In some embodiments, the genomic modification results in a substitution of a histidine at the amino acid at position 4, an arginine at the amino acid at position 49, a proline at the amino acid at position 49, an alanine at the amino acid at position 52, an alanine at the amino acid at position 53, a proline at the amino acid at position 53, an alanine at the amino acid at position 120, or a lysine at the amino acid at position 124 of a wildtype gene encoding CD47 or at a corresponding position in a homologous CD47 gene.

In some embodiments, the epitope is a region of CD34 bound by anti-CD34 antibody QBend10, anti-CD34 antibody 561, or fragments thereof. In some embodiments, the genomic modification results in a deletion, a substitution, an insertion, or an inversion of one or more of the amino acids at positions 42, 45, 46, 47, 49, 50, 51, 54, or 55 of a wildtype gene encoding CD34 or at corresponding positions in a homologous CD34 gene. In some embodiments, the one or more substitutions are conservative substitutions. In some embodiments, the genomic modification results in a substitution of one or more of the amino acids at positions 42, 45, 46, 47, 49, 50, 51, 54, or 55 of a wildtype gene encoding CD34 or at corresponding positions in a homologous CD34 gene. In some embodiments, the genomic modification results in a substitution of an alanine at the amino acid at any one or more of positions 45, 46, 50, 51, 54, 55 of a wildtype gene encoding CD34 or at a corresponding position in a homologous CD34 gene. In some embodiments, the genomic modification results in a substitution of phenylalanine at the amino acid of position 46, lysine at the amino acid of position 47, glutamic acid at the amino acid position 47, phenylalanine at amino acid position 49, or serine at amino acid position 49 of a wildtype gene encoding CD34 or at a corresponding position in a homologous CD34 gene.

In another aspect, the disclosure is directed to a method, comprising administering to a subject in need thereof a population of genetically engineered hematopoietic cells, or descendants thereof, described herein. In some embodiments, a method of the disclosure further comprises administering an effective amount of the agent that specifically binds the lineage-specific cell-surface antigen. In some embodiments, the subject has a hematopoietic malignancy.

In some embodiments, the agent is a single-chain antibody fragment (scFv). In some embodiments, the agent is an antibody or an antibody-drug conjugate (ADC). In some embodiments, the agent is an immune cell expressing a chimeric antigen receptor that comprises the antigen-binding fragment.

In some embodiments, the immune cells are T cells. In some embodiments, the T cells express CD3, CD4, and/or CD8.

In some embodiments, the chimeric antigen receptor further comprises: a hinge domain, a transmembrane domain, at least one co-stimulatory domain, a cytoplasmic signaling domain, or a combination thereof. In some embodiments, the chimeric antigen receptor comprises at least one co-stimulatory signaling domain, which is derived from a co-stimulatory receptor selected from the group consisting of CD27, CD28, 4-1BB, OX40, CD30, ICOS, CD2, CD7, LIGHT, NKG2C, B7-H3, GITR, HVEM, and a combination thereof. In some embodiments, the chimeric antigen receptor comprises a cytoplasmic signaling domain, which is from CD3ζ. In some embodiments, the chimeric antigen receptor comprises a hinge domain, which is from CD8α or CD28.

In some embodiments, the agent comprises: murine anti-CD123 antibody 7G3, a humanized variant thereof (e.g., antibody CSL-362), or talacotuzumab; murine anti-CD38 antibody HB7, a humanized variant thereof, or daratumumab; B43; blinatumomab; FMC63, or HIB19; or anti-CD47 antibody B6H12 or 2D3; or anti-CD34 antibody QBend10 or 561; or anti-CD5 antibody H65.

In some embodiments, the hematopoietic malignancy is Hodgkin's lymphoma, non-Hodgkin's lymphoma, leukemia, multiple myeloma (MM), myelodysplastic syndrome (MDS), or blastic plasmacytoid dendritic cell neoplasm (BPDCN). In some embodiments, the hematopoietic malignancy is acute myeloid leukemia, B-cell acute lymphoblastic leukemia (B-ALL), chronic myelogenous leukemia, acute lymphoblastic leukemia, or chronic lymphoblastic leukemia. In some embodiments, the hematopoietic malignancy is B-cell acute lymphoblastic leukemia (B-ALL). In some embodiments, the hematopoietic malignancy is acute myeloid leukemia (AML). In some embodiments, the hematopoietic malignancy is multiple myeloma (MM). In some embodiments, the hematopoietic malignancy is myelodysplastic syndrome (MDS).

In another aspect, the disclosure is directed to a method comprising: genetically modifying a hematopoietic cell to introduce a genomic modification in a gene encoding a lineage-specific cell-surface antigen, wherein the genomic modification alters the amino acid sequence of an epitope that is recognized by an agent that specifically binds the lineage-specific cell-surface antigen resulting in a modified lineage-specific cell surface antigen, wherein the modified lineage-specific cell-surface antigen is characterized by reduced binding or no binding of the agent, thereby producing a genetically engineered hematopoietic cell having reduced binding or no binding to an agent targeting the lineage-specific cell-surface antigen. In some embodiments, a method of the disclosure further comprises: providing a hematopoietic cell.

In some embodiments, the genetically engineered hematopoietic cell is a genetically engineered hematopoietic cell described herein.

In some embodiments, genetically modifying the hematopoietic cell comprises contacting the cell with: (a) a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a gene encoding a lineage-specific cell-surface antigen (e.g., a sequence encoding an epitope bound by an agent that specifically binds the lineage-specific cell-surface antigen) in the genome of the hematopoietic cell; and (b) a template polynucleotide. In some embodiments, the contacting further comprises contacting the hematopoietic cell with: (c) one or both of: an expansion agent; and a homology-directed repair (HDR) promoting agent. In some embodiments, the CRISPR/Cas system creates a double-stranded break (DSB) in the gene encoding the lineage-specific cell-surface antigen in the genome of the hematopoietic cell.

In some embodiments, the template polynucleotide is a single-stranded donor oligonucleotide (ssODN) or a double-stranded donor oligonucleotide (dsODN). In some embodiments, the template polynucleotide hybridizes to a genomic sequence flanking the DSB in the gene encoding the lineage-specific cell-surface antigen and integrates into the gene encoding the lineage-specific cell-surface antigen. In some embodiments, the template polynucleotide comprises a donor sequence, a first flanking sequence which is homologous to a genomic sequence upstream of the DSB in the gene encoding the lineage-specific cell-surface antigen and a second flanking sequence which is homologous to a genomic sequence downstream of the DSB in the gene encoding the lineage-specific cell-surface antigen. In some embodiments, the donor sequence of the template polynucleotide is integrated into the genome of the hematopoietic cell by homology-directed repair (HDR).

In some embodiments, the expansion agent comprises SR1 and UM171. In some embodiments, the HDR promoting agent comprises at least one of SCR7, NU7441, Rucaparib, and RS-1.

In some embodiments, the ssODN is between 50 to 200 nucleotides in length. In some embodiments, the ssODN is 120 nucleotides in length.

In some embodiments, contacting comprises contacting a population of hematopoietic cells. In some embodiments, a method described herein further comprises sorting the population of hematopoietic cells. In some embodiments, sorting comprises selecting for viable hematopoietic cells. In some embodiments, sorting comprises selecting for hematopoietic cells that integrated the donor sequence into their genome. In some embodiments, sorting comprises Fluorescence Activated Cell Sorting (FACS). In some embodiments, sorting comprises selecting for viable long term engrafting HSCs.

In some embodiments, the editing efficiency in the population of hematopoietic cells is at least 30, at least 40, at least 50, at least 60, at least 70, at least 80, at least 90, at least 95, or at least 99%. In some embodiments, the percent viability in the population of hematopoietic cells is at least 50, at least 60, at least 70, at least 80, at least 90, at least 95, or at least 99%. In some embodiments, the efficiency of HDR is 50% or higher. In some embodiments, the efficiency of HDR is 60% or higher. In some embodiments, the efficiency of HDR is 80% or higher.

In some embodiments, the lineage-specific cell-surface antigen is selected from the group consisting of CD33, CD123, CD19, CLL-1, CD30, CD5, CD6, CD7, CD34, CD38, CD47, EMR2/CD312, and BCMA. In some embodiments, the lineage-specific cell-surface antigen is CD123. In some embodiments, the lineage-specific cell-surface antigen is EMR2.

In some embodiments, the gRNA comprises a nucleic acid sequence selected from the group consisting of SEQ ID NOs: 3, 6, 9, and 12.

In some embodiments, the first flanking sequence is homologous to a first portion of the CD123 gene and the second flanking sequence is homologous to a second portion of the CD123 gene. In some embodiments, the first portion of the CD123 gene comprises a portion of exon 3 or a sequence proximal thereto. In some embodiments, the first portion of the CD123 gene comprises a portion of exon 4 or a sequence proximal thereto. In some embodiments, the second portion of the CD123 gene comprises a portion of exon 3 or a sequence proximal thereto. In some embodiments, the second portion of the CD123 gene comprises a portion of exon 4 or a sequence proximal thereto. In some embodiments, the first portion and second portion are not identical. In some embodiments, the donor sequence comprises a sequence corresponding to the codon(s) encoding 1, 2, 3, 4, or 5 of the amino acids at positions 51, 59, 61, 82, or 84 of a wildtype gene encoding CD123. In some embodiments, the first flanking sequence comprises a flanking sequence set forth in any one of SEQ ID NOs: 93-99. In some embodiments, the second flanking sequence comprises a flanking sequence set forth in any one of SEQ ID NOs: 93-99. In some embodiments, the donor sequence comprises a donor sequence set forth in any one of SEQ ID NOs: 93-99. In some embodiments, the template polynucleotide comprises a nucleic acid sequence selected from the group consisting of SEQ ID NOs: 93-99.

In some embodiments, the lineage-specific cell-surface antigen is CD38. In some embodiments, the gRNA comprises a nucleic acid sequence selected from the group consisting of SEQ ID NOs: 18, 21, 24, 27, 30, 33, 36, 39, 42, 45, 48, 51, 54, 57, and 60. In some embodiments, the first flanking sequence is homologous to a first portion of the CD38 gene and the second flanking sequence is homologous to a second portion of the CD38 gene. In some embodiments, the first portion of the CD38 gene comprises a portion of exon 7 or a sequence proximal thereto. In some embodiments, the second portion of the CD38 gene comprises a portion of exon 7 or a sequence proximal thereto. In some embodiments, the first portion and second portion are not identical. In some embodiments, the donor sequence comprises a sequence corresponding to the codon(s) encoding 1, 2, 3, 4, or 5 of the amino acids at positions 270-274 of a wildtype gene encoding CD38.

In some embodiments, the lineage-specific cell-surface antigen is CD19. In some embodiments, the gRNA comprises a nucleic acid sequence selected from the group consisting of SEQ ID NOs: 66, 69, 72, 75, 78, 81, and 84. In some embodiments, the first flanking sequence is homologous to a first portion of the CD19 gene and the second flanking sequence is homologous to a second portion of the CD19 gene. In some embodiments, the first portion of the CD19 gene comprises a portion of exon 2 or a sequence proximal thereto. In some embodiments, the first portion of the CD19 gene comprises a portion of exon 4 or a sequence proximal thereto. In some embodiments, the second portion of the CD19 gene comprises a portion of exon 2 or a sequence proximal thereto. In some embodiments, the second portion of the CD19 gene comprises a portion of exon 4 or a sequence proximal thereto. In some embodiments, the first portion and second portion are not identical. In some embodiments, the donor sequence comprises a sequence corresponding to the codon(s) encoding 1, 2, 3, 4, 5, 6, 7, 8, 9, 10, 11, 12, 13, 14, 15, 16, 17, 18, 19, or 20 of the amino acids at positions 216-224 or 218-238 of a wildtype gene encoding CD19.

In some embodiments, the genomic modification results in expression of a variant form of the lineage-specific cell-surface antigen that is not recognized by the agent. In some embodiments, the genomic modification results in expression of a variant form of the lineage-specific cell-surface antigen that is recognized by a second agent that specifically binds to a different region of the lineage-specific cell-surface antigen than the agent that binds the epitope.

In some embodiments, the Cas nuclease is a Cas9 nuclease. In some embodiments, the Cas nuclease is a Streptococcus pyogenes Cas9 (spCas9) nuclease. In some embodiments, the Cas nuclease is a Staphylococcus aureus Cas9 (saCas9) nuclease. In some embodiments, the Cas nuclease is a Cas12a nuclease. In some embodiments, the Cas nuclease is a Cas12b nuclease.

In some embodiments, the contacting comprises introducing the CRISPR/Cas system into the cell in the form of a pre-formed ribonucleoprotein (RNP) complex. In some embodiments, the ribonucleoprotein complex is introduced into the hematopoietic cell via electroporation. In some embodiments, the template polynucleotide and CRISPR/Cas system are electroporated into the cell simultaneously.

In another aspect, the disclosure is directed to a method of producing a genetically engineered hematopoietic stem or progenitor cell, or a plurality thereof, comprising at least one nucleotide substitution in a gene encoding a lineage-specific cell-surface antigen, wherein the method comprises introducing into a hematopoietic stem or progenitor cell a guide RNA (gRNA) comprising a targeting domain targeting a nucleotide sequence within the genome of the hematopoietic stem or progenitor cell, and a base editor comprising a catalytically impaired Cas9 endonuclease fused to a cytosine (CBE) or adenosine deaminase (CBE), thereby producing the genetically engineered hematopoietic stem or progenitor cell or a plurality thereof.

In some embodiments, the at least one substitution produces a missense variant in the gene encoding the lineage-specific cell-surface antigen. In some embodiments, the at least one substitution produces an alteration in the translation start site of the gene encoding the lineage-specific cell-surface antigen. In some embodiments, the at least one substitution produces a splice region variant in the gene encoding the lineage-specific cell-surface antigen. In some embodiments, the substitution results in reduced or eliminated expression of a gene encoding a wild-type version of the lineage-specific cell-surface antigen.

In some embodiments, the gene encoding the lineage-specific cell-surface antigen is selected from the group consisting of CD123, CD47, CD34, CD38, CD19, CD33, CLL-1, CD30, CD5, CD6, CD7, and BCMA. In some embodiments, the gene encoding the lineage-specific cell-surface antigen is selected from the group consisting of CD123, CD47, CD34, CD38, CD19, and CD5. In some embodiments, the gene encoding the lineage-specific cell-surface antigen is CD123. In some embodiments, the gene encoding the lineage-specific cell-surface antigen is CD47. In some embodiments, the gene encoding the lineage-specific cell-surface antigen is CD34. In some embodiments, the gene encoding the lineage-specific cell-surface antigen is CD38. In some embodiments, the gene encoding the lineage-specific cell-surface antigen is CD19. In some embodiments, the gene encoding the lineage-specific cell-surface antigen is CD5.

In some embodiments, the gRNA comprises a nucleotide sequence set forth in any one of Tables 1-13. In some embodiments, the gRNA comprises a nucleotide sequence set forth in any one of SEQ ID NOs: 1-12, 16-60, 64-84, 100-181, 195, 196, and 204-423.

In some embodiments, the catalytically impaired Cas9 nuclease is a SpRY Cas9. In some embodiments, the catalytically impaired Cas9 nuclease is a SpG Cas9. In some embodiments, the base editor is introduced into the cell as an mRNA. In some embodiments, the base editor and gRNA are introduced into the cell via electroporation.

In some embodiments, the method further comprises sorting the genetically engineered hematopoietic stem or progenitor cell, or plurality thereof, via fluorescence-activated cell sorting (FACS).

In another aspect, the disclosure is directed to a genetically engineered hematopoietic cell, where the cell is obtained or obtainable by a method described herein.

In another aspect, the disclosure is directed to a population of genetically engineered hematopoietic cells comprising a plurality of the genetically engineered hematopoietic cells described herein.

In another aspect, the disclosure is directed to a pharmaceutical composition comprising a genetically engineered hematopoietic cell, or descendant thereof, described herein or a population of genetically engineered hematopoietic cells described herein.

In another aspect, the present disclosure is directed to a method of treating a hematopoietic disease, comprising administering to a subject in need thereof an effective amount of a genetically engineered stem or progenitor cell, a cell population thereof, or a pharmaceutical composition thereof described herein. In some embodiments, the hematopoietic disease is a hematopoietic malignancy.

In some embodiments, the method further comprises administering an effective amount of an agent that targets a wildtype version of the lineage-specific cell-surface antigen. In some embodiments, the agent comprises an antibody or antigen-binding fragment that binds to the wildtype version of the lineage-specific cell-surface antigen. In some embodiments, the antibody is selected from the group consisting of an anti-CD123 antibody 7G3, talacotuzumab, anti-CD38 antibody HB7, daratumumab, anti-CD38 antibody B43, blinatumomab, anti-CD19 antibody FMC63, anti-CD19 antibody HIB19, anti-CD47 antibody B6H12, anti-CD47 antibody 2D3, anti-CD34 antibody QBend10, anti-CD34 antibody 561, and anti-CD5 antibody H65.

In some embodiments, the agent is an immune cell. In some embodiments, the immune cell is a cytotoxic T cell. In some embodiments, the cytotoxic T cell expresses a chimeric antigen receptor (CAR) which comprises the antibody or antigen-binding fragment that binds the wildtype version of the lineage-specific cell-surface antigen.

In some embodiments, the genetically engineered stem or progenitor cell, the immune cell, or both, are allogenic. In some embodiments, the genetically engineered stem or progenitor cell, the immune cell, or both, are autologous.

In some embodiments, the subject is a human patient having Hodgkin's lymphoma, non-Hodgkin's lymphoma, leukemia, acute myeloid leukemia (AML), chronic myelogenous leukemia, acute lymphoblastic leukemia, or chronic lymphoblastic leukemia.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIGS. 1A-1D show an exemplary strategy for CD123 editing for specific targeting of disease cells using monoclonal antibody therapy. FIG. 1A shows the crystal structure of CD123 bound to the anti-CD123 antibody CSL-362 (JNJ-56022473, 7G3) (derived from PDB: 4JZJ). FIG. 1B shows the crystal structure of the N-terminal domain (NTD) of CD123 bound to heavy and light chains of CSL-362 (JNJ-56022473; 7G3). Amino acid residues S59, E51 and R84 of CD123 are labeled as residues important for the binding of CSL-362 antibody to CD123. The crystal structure is derived from PDB: 4JZJ. FIG. 1C shows a list of the residues thought to be important for CD123 binding to CSL-362 (7G3), including E51, S59, P61, T82, and R84. FIG. 1D shows a diagram for an exemplary process to generate the CD123-targeting antibody talacotuzumab (JNJ-56022473) from the mouse anti-CD123 antibody 7G3 to the humanized version, CSL-362.

FIGS. 2A-2B show identification of tolerable CD123 variants. FIG. 2A shows annotation of CD123 variants in the human genome using the Genome Aggregation Database (gnomAD). FIG. 2B shows amino acid sequence alignment of human CD123 with various non-human primate CD123 sequences.

FIGS. 3A-3F show an exemplary strategy for epitope modification and downstream mutant characterization. FIG. 3A shows an exemplary modification mutagenesis strategy. FIG. 3B shows an exemplary strategy for ectopic expression and screening of CD123 mutants. FIG. 3C shows exemplary microscopy analyses used to screen CD123 mutants. FIGS. 3D-3E show flow cytometry analysis of antibody binding to CD123 modified epitopes (i.e. CD123 mutants) (APC=allophycocyanin; PE=phycoerythrin). FIG. 3D shows a flow cytometry analysis of CD123 antibody clones 6H6 (left panel) and 9F5 (right panels) binding to the indicated CD123 modified epitopes using a PE fluorophore. FIG. 3E shows a comparison of flow cytometry analysis of CD123 antibody clone 7G3 binding to CD123 modified epitopes using APC (left panel) or PE (right panel) fluorophores. FIG. 3F shows the percentage of CD123+ cells only in the PE+ (left plots) versus the GFP+ and PE+ (right plots) cell populations as a plasmid expression control for 6H6/9F5 antibody clones (top plots) and the 7G3 clone (bottom plots).

FIGS. 4A and 4B shows flow cytometry analysis of the effect of IL3 on antibody binding to CD123 and epitope-modified CD123. FIG. 4A shows IL3 does not effect antibody binding to CD123 for either antibody clone 6H6 (left plot) or clone 7G3 (right plot). FIG. 4B shows IL3 does not effect antibody binding to epitope-modified (E51K) CD123 for either antibody clone 6H6 (left plot) or clone 7G3 (right panel).

FIGS. 5A-5E show characterization of daratumumab (Genmab/JNJ) binding to cyclic ADP-ribose hydrolase 1 type II transmembrane glycoprotein (also referred to as CD38). FIG. 5A shows a diagram annotating structural features of CD38 isoforms with daratumumab binding sites indicated. FIG. 5B shows a crystal structure of daratumumab (top polypeptide) docked on CD38 (lower polypeptide) indicating residues important for binding (middle amino acid segment). FIG. 5C shows flow cytometry analysis of anti-CD38 antibody binding to CD38 wildtype and the indicated CD38 mutants. FIG. 5D shows flow cytometry analysis of HB7 antibody clone (right panel) binding to the indicated CD38 mutants or CD38 wildtype as compared to a HIT2 antibody clone control (left panel). FIG. 5E shows flow cytometry analysis of HB7 antibody clone (right panel) binding to cells expressing the indicated CD38 Glutamine 272 mutants (Q272A, Q272H, or Q272R) or CD38 wildtype as compared to a HIT2 antibody clone control (left panel).

FIG. 6 shows a diagram of an exemplary experimental design for editing CD34+ cells using ssODN-based homology-directed repair (HDR) via CRISPR.

FIG. 7 shows a diagram of an exemplary HDR approach used to edit the interleukin 3 receptor alpha type 1 cytokine receptor (CD123) gene indicating sites targeted by exemplary guide RNAs (gRNAs) and donor oligonucleotides (ssODNs).

FIGS. 8A-8C shows flow cytometry analysis of HDR-edited CD34+ hematopoietic stem cells (HSCs) stained with anti-CD123 antibodies 7G3 (light grey peaks) and 6H6 (dark grey peaks) wherein the gRNAs (g31 and g29) and ssODNs (ss31 and ss29) correspond to the oligonucleotides mapped in FIG. 7. FIG. 8A shows flow cytometry analysis of healthy cells from donor 1 that were treated with either mock electroporation (EP) (no electroporation negative control), ss29/or ss31 alone (negative ssODN control), Cas9g29 or Cas9g31 alone (positive control for NHEJ cutting), or both. FIG. 8B shows flow cytometry analysis of healthy cells from donor 2. FIG. 8C shows a histogram representation of the quantification of the data in FIGS. 8A and 8B (y-axis shows the % staining of CD123+ cells with 6H6 or 7G3 gated based on isotype control).

FIGS. 9A and 9B show an exemplary experimental approach for editing and characterizing HDR-edited CD123 mutants. FIG. 9A shows a diagram of an exemplary experimental design for HDR-editing of cells from three CD34+ donors (Donor 1, Donor 2, TIB-202 (THP-1 cells)). FIG. 9B shows radiation-assisted amplification sequencing (RAMP-Seq) data as a quality check for DNA sequencing control.

FIGS. 10A-10C shows editing outcomes from HDR-targeting of AML donor cells. FIG. 10A shows a diagram of possible genomic changes following HDR-editing procedures wherein non-homologous end-joining (NHEJ) outcomes may result in deletions in the genomic locus, “imperfect” editing outcomes may result in a combination of deletions and incorporation of mutations encoded by single-stranded donor oligonucleotide (ssODN), and HDR outcomes result in site-specific changes in the genomic locus using the donor DNA template to direct repair of the cleaved site. FIG. 10B shows an exemplary editing percentage summary as a result of targeting donor cells with g29, ss29, or g29+ss29 (G116) or g31, ss31, or g31+ss31 TIB-202 refers to a CD123+ control cell line. FIG. 10C shows flow cytometry analysis of CD34 donor 2 cells bearing knockout (KO) and HDR products via staining with an antibody which does not recognize the HDR-edited epitope of CD123 (antibody clone 6H6) and an antibody that recognizes the HDR-edited epitope (antibody clone 7G3).

FIGS. 11A-11D show results from epitope modification of CD19. FIG. 11A shows flow cytometry analyses of anti-CD19 antibody clone FMC63 (right panel) binding to HEK293T cells expressing the indicated CD19 mutations, as compared to HIB 19 control antibody (left panel). FIG. 11B shows flow cytometry analyses of anti-CD19 clone FMC63 (left panel) binding to HEK293T cells expressing the indicated CD19 mutations, as compared to HIB19 control antibody (right panel). FIG. 11C shows exemplary gRNAs for epitope modification of CD19 and the expected substitution mutation(s). FIG. 11D shows Sanger sequencing and flow cytometry analyses of Raji cells expressing CD19 epitope modifications at amino acids at positions 162, 163, and/or 164 of CD19. The flow cytometry was performed with the anti-CD19 antibody clone FMC63.

FIGS. 12A and 12B show results from epitope modification of CD47. FIG. 12A shows flow cytometry analyses of anti-CD47 clone B6H12 (left panel) binding to HEK293T cells expressing the indicated CD47 mutations as compared to 2D3 control antibody (right panel). FIG. 12B shows a quantification of flow cytometry analyses of anti-CD47 clone B6H12 binding to HEK293T cells expressing the indicated CD47 mutations.

FIGS. 13A-13F show results from epitope modification of CD34. FIG. 13A shows flow cytometry analyses of anti-CD34 clones QBend10 (left panel) and 561 (right panel) binding to HEK293T cells expressing the indicated CD34 mutations. FIG. 13B shows flow cytometry analyses of anti-CD34 clones QBend10 (left panel) and 561 (right panel) binding to HEK293T cells expressing the indicated CD34 mutations. FIG. 13C shows quantification of the flow cytometry data displayed in FIG. 13B. FIG. 13D shows exemplary gRNAs for epitope modification of CD34 using base editors CBEs or ABEs in hematopoietic stem progenitor cells (HSPCs). FIG. 13E shows flow cytometry analyses of anti-CD34 clones QBend10 (left panel) and 561 (right panel) binding to CD34+ donor cells following transfection with the indicated gRNAs and either CBE or control RNP (Cas9 and CD34 gRNA). FIG. 13F shows quantification of the flow cytometry data in FIG. 13E.

FIG. 14 shows a crystal structure of CD5 indicating the extracellular and transmembrane domains (TMD) in addition to the binding region for anti-CD5 monoclonal antibody clone H65 which is located in domain 1.

FIGS. 15A-15B show results from epitope modification of EMR2. FIG. 15A shows a crystal structure of EMR2 (Source: alphafold.ebi.ac.uk/entry/AOJNV7) indicating the EGF domains in addition to the binding region for anti-EMR2 monoclonal antibody clone 2A1 which is located in Helix 1 of the GAIN domain/GPS. FIG. 15B shows flow cytometry analyses of Flag L5 control antibody (left panel) binding to HEK293T cells expressing the indicated EMR2 mutations as compared to anti-EMR2 clone 2A1 antibody (right panel).

### DETAILED DESCRIPTION

Some aspects of this disclosure provide compositions and methods for genetically engineering a cell (e.g., a hematopoietic cell, e.g., hematopoietic stem cells (HSCs)) to modify a gene encoding a lineage-specific cell-surface antigen to alter the amino acid sequence of an epitope of the lineage-specific cell-surface antigen recognized by an agent. Some aspects of this disclosure are based, at least in part, on the identification and characterization of modified epitopes which reduce or abolish binding of the agent. Some aspects of this disclosure provide strategies, and treatment modalities related to genetically modified/engineered cells that express a modified epitope, variant form of a lineage-specific cell-surface antigen targeted by a therapeutic agent, e.g., an immunotherapeutic agent. The genetically engineered cells provided herein are useful, for example, to mitigate, or avoid altogether, certain undesired effects, for example, any on-target, off-disease cytotoxicity, associated with certain immunotherapeutic agents.

Such undesired effects associated with certain immunotherapeutic agents may occur, for example, when healthy cells within a subject in need of an immunotherapeutic intervention express an antigen targeted by an immunotherapeutic agent. For example, a subject may be diagnosed with a malignancy associated with an elevated level of expression of a specific antigen, which is not typically expressed in healthy cells, but may be expressed at relatively low levels in a subset of non-malignant cells within the subject. Alternatively, or in addition, a subject may be in need of ablation of cells expressing a lineage-specific cell-surface antigen, such as CD33, CD123, CD19, CLL-1, CD30, CD5, CD6, CD7, CD34, CD38, CD47, EMR2 (CD312), and BCMA. Administration of an immunotherapeutic agent, e.g., a CAR-T cell therapeutic or a therapeutic antibody or antibody-drug-conjugate (ADC) targeting the antigen, to the subject may result in efficient killing of the target cells, e.g., of malignant cells characterized by expressing the respective lineage-specific cell-surface antigen, but may also result in ablation of non-target cells expressing the antigen in the subject, e.g., of hematopoietic cells characterized by expressing the respective lineage-specific cell-surface antigen. This on-target, off-disease cytotoxicity can result in significant side effects and, in some cases, abrogate the use of an immunotherapeutic agent altogether.

The compositions, methods, strategies, and treatment modalities provided herein address the problem of on-target, off-disease cytotoxicity of certain immunotherapeutic agents. Some aspects of this disclosure provide genetically engineered cells comprising a modification in their genome that results in expression of a modified lineage-specific cell-surface antigen that exhibits decreased or no binding to an agent (e.g., an immunotherapeutic agent) that specifically binds to the lineage-specific cell-surface antigen. In some embodiments, such genetically engineered cells, and their progeny, are not targeted by the agent or are only targeted to a significantly reduced degree as compared to non-engineered cells of the same cell type and are not subject to cytotoxicity effected by the immunotherapeutic agent or subject to a reduced degree of cytotoxicity. In some embodiments, a genetically engineered cell of the disclosure is produced using homology-directed repair (HDR), which allows targeted integration of sequences from a template polynucleotide at a target sequence specified by homology of portions of a template polynucleotide to the target sequence. In some embodiments, a genetically engineered cell of the disclosure is produced using base editing, which allows targeted substitution, insertion, and deletion of sequences at a target sequence specified by gRNAs directed against the target sequence. Accordingly, some aspects of the present disclosure provides genetically engineered cells comprising a modified gene encoding a lineage-specific cell surface antigen, methods of treating a subject in need thereof by administering such cells to the subject, compositions, e.g., genetic modification mixtures, for use in genetically engineering cells, methods for genetically engineering cells to comprise modified genes encoding epitope-modified lineage-specific cell surface antigens, and other compositions (e.g., pharmaceutical compositions) related to any thereof.

### Cells

Some aspects of the present disclosure provide methods and compositions for genetically modifying cells, genetically modified cells produced by such methods, and methods of using said modified cells (e.g., to treat a subject in need thereof). In some embodiments, the genetically modified cell is a hematopoietic cell. In some embodiments, the genetically modified hematopoietic cell is a hematopoietic stem cell (HSC) or hematopoietic progenitor cell (HPC). In some embodiments, a method or composition described herein is used to genetically modify a hematopoietic cell (e.g., an HSC or HPC) e.g., in a gene encoding a lineage-specific cell-surface antigen.

Some aspects of this disclosure provide genetically modified hematopoietic cells and uses thereof. In some embodiments, such a cell is created by contacting the cell with a CRISPR/Cas system (e.g., a Cas nuclease and/or gRNA) and a template polynucleotide, or in some embodiments, the cell is a daughter cell of the cell that was contacted with the CRISPR/Cas system and a template polynucleotide. In some embodiments, such a cell is created by contacting the cell with a preformed ribonucleoprotein complex comprising a base editor and a gRNA, or in some embodiments, the cell is daughter cell of the cell that was contacted with the ribonucleoprotein complex. In some embodiments, a cell described herein (e.g., a genetically engineered HSC or HPC) is capable of populating the HSC or HPC niche and/or of reconstituting the hematopoietic system of a subject. In some embodiments, a cell described herein (e.g., an HSC or HPC) is capable of one or more of (e.g., all of): engrafting in a human subject, producing myeloid lineage cells, and producing lymphoid lineage cells. In some preferred embodiments, a genetically engineered hematopoietic cell provided herein, or its progeny, can differentiate into all blood cell lineages, preferably without any differentiation bias as compared to a hematopoietic cell of the same cell type, but not comprising the respective HDR-mediated genomic modification. In some embodiments, the genetically engineered cells, e.g., genetically engineered HSCs, are autologous to a subject, e.g., a subject to be treated for a disease, e.g., a cancer, auto-immune disease, or genetic disease. In some embodiments, the genetically engineered cells, e.g. the genetically engineered HSCs, are derived from a subject with a cancer, auto-immune disease, or genetic disease or at risk of developing a cancer, auto-immune disease, or genetic disease (i.e., autologous cells). In some embodiments, the HSCs to be genetically engineered using the disclosed methods are obtained from a subject who is not the subject to whom the cells will be administered, and are referred to as allogeneic cells. In some embodiments, the HSCs are derived from a donor having a HLA haplotype that is matched with the HLA haplotype of the subject. Human Leukocyte Antigen (HLA) encodes major histocompatibility complex (MHC) proteins in humans. MHC molecules are present on the surface of antigen-presenting cells as well as many other cell types and present peptides of self and non-self (e.g., foreign) antigens for immunosurveillance. However, HLA are highly polymorphic, which results in many distinct alleles. Different (foreign, non-self) alleles may be antigenic and stimulate robust adverse immune responses, particularly in organ and cell transplantation. HLA molecules that are recognized as foreign (non-self) can result in transplant rejection. In some embodiments, it is desirable to derive HSCs from a donor that has the same HLA type as the patient to reduce the incidence of rejection.

The HLA loci of a donor subject may be typed to identify an individual as a HLA-matched donor for the subject. Methods for typing the HLA loci will be evident to one of ordinary skill in the art and include, for example, serology (serotyping), cellular typing, gene sequencing, phenotyping, and PCR methods. A HLA from a donor is considered “matched” with the HLA of the subject if the HLA loci of the donor and the subject are identical or sufficiently similar such that an adverse immune response is not expected.

In some embodiments, a genetically engineered hematopoietic cell of the disclosure comprises a genetic modification proximal to a PAM sequence, e.g., a PAM sequence in a gene encoding a lineage-specific cell-surface antigen (e.g., a sequence encoding an epitope bound by an agent that specifically binds the lineage-specific cell-surface antigen). In some embodiments, the genetic modification comprises integration of a donor sequence. In some embodiments, the integration of a donor sequence results in an insertion mutation or a substitution mutation. In some embodiments, a donor sequence is inserted 5′ of a PAM sequence, e.g., of a Cas9 PAM sequence. In some embodiments, a donor sequence is inserted 5′ of a PAM sequence. In some embodiments, a donor sequence is inserted 1, 2, 3, 4, 5, 6, 7, 8, 9, 10, 11, 12, 13, 14, 15, 16, 17, 18, 19, or 20 nucleotides 5′ of a PAM sequence. In some embodiments, a donor sequence is inserted 1-10, 1-8, 1-6, 1-4, 2-10, 2-8, 2-6, 2-4, 4-10, 4-8, 4-6, 6-10, 6-8, 8-10, 10-20, 15-20, 16-20, 17-20, 18-20, 19-20, 16-19, 17-19, 18-19, 16-18, or 17-18 nucleotides 5′ of a PAM sequence, e.g., 2, 3, or 4 nucleotides 5′ of a PAM sequence. In some embodiments, a donor sequence is inserted 3′ of a PAM sequence, e.g., of a Cas9 PAM sequence. In some embodiments, a donor sequence is inserted 1, 2, 3, 4, 5, 6, 7, 8, 9, 10, 11, 12, 13, 14, 15, 16, 17, 18, 19, or 20 nucleotides 3′ of a PAM sequence. In some embodiments, a donor sequence is inserted 1-10, 1-8, 1-6, 1-4, 2-10, 2-8, 2-6, 2-4, 4-10, 4-8, 4-6, 6-10, 6-8, 8-10, 10-20, 15-20, 16-20, 17-20, 18-20, 19-20, 16-19, 17-19, 18-19, 16-18, or 17-18 nucleotides 3′ of a PAM sequence, e.g., 17, 18, or 19 nucleotides 3′ of a PAM sequence.

In some embodiments, a genetically engineered hematopoietic cell comprises a genetic modification corresponding to integration of a donor sequence (e.g., from a template polynucleotide described herein) into a gene encoding a lineage-specific cell-surface antigen in the hematopoietic cell. In some embodiments, the genetic modification corresponds to a position or positions where the donor sequence differs from the sequence of the gene encoding a lineage-specific cell-surface antigen. In some embodiments, integration of the donor sequence results in modification at 1, 2, 3, 4, 5, 6, 7, 8, 9, or 10 bases (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 bases) in the gene encoding a lineage-specific cell-surface antigen. In some embodiments, integration of the donor sequence results in an insertion of 1, 2, 3, 4, 5, 6, 7, 8, 9, or 10 bases (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 bases) in the gene encoding a lineage-specific cell-surface antigen. In some embodiments, integration of the donor sequence results in substitution of 1, 2, 3, 4, 5, 6, 7, 8, 9, or 10 bases (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 bases) in the gene encoding a lineage-specific cell-surface antigen. In some embodiments, integration of the donor sequence results in modification at a number of positions in the gene encoding a lineage-specific cell-surface antigen corresponding to up to 1, 5, 10, 15, 20, 25, 30, 35, 40, 45, 50, 55, 60, 65, 70, 75, 80, 85, 90, 95, or 100% of the length of the donor sequence. In some embodiments, integration of the donor sequence results in insertion of a number of bases in the gene encoding a lineage-specific cell-surface antigen corresponding to up to 1, 5, 10, 15, 20, 25, 30, 35, 40, 45, 50, 55, 60, 65, 70, 75, 80, 85, 90, 95, or 100% of the length of the donor sequence. In some embodiments, the donor sequence is 1-100, 1-80, 1-60, 1-40, 1-20, 1-15, 1-10, 1-9, 1-8, 1-7, 1-6, 1-5, 1-4, 1-3, 1-2, 5-100, 5-80, 5-60, 5-40, 5-20, 5-15, 5-10, 5-9, 5-8, 5-7, 5-6, 10-100, 10-80, 10-60, 10-40, 10-20, 10-15, 20-100, 20-80, 20-60, 20-40, 60-100, or 60-80 nucleotides in length. In some embodiments, a donor sequence is no more than 100, no more than 90, no more than 80, no more than 70, no more than 60, no more than 50, no more than 45, no more than 40, no more than 35, no more than 30, no more than 25, no more than 20, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 bases long. In some embodiments, a donor sequence is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 bases long. In some embodiments, integration of the donor sequence results in modification of no more than 1, no more than 2, no more than 3, no more than 4, no more than 5, no more than 6, no more than 7, no more than 8, no more than 9, or no more than 10 bases in the gene encoding a lineage-specific cell-surface antigen. In some embodiments, integration of the donor sequence results in substitution at no more than 1, no more than 2, no more than 3, no more than 4, no more than 5, no more than 6, no more than 7, no more than 8, no more than 9, or no more than 10 bases in the gene encoding a lineage-specific cell-surface antigen. In some embodiments, integration of the donor sequence results in insertion of no more than 1, no more than 2, no more than 3, no more than 4, no more than 5, no more than 6, no more than 7, no more than 8, no more than 9, or no more than 10 bases in the gene encoding a lineage-specific cell-surface antigen.

In some embodiments, integration of the donor sequence into the genetically engineered hematopoietic cell alters the amino acid sequence of an epitope of a lineage-specific cell-surface antigen, wherein the epitope is recognized by an agent that specifically binds the lineage-specific cell-surface antigen. In some embodiments, the integrated donor sequence comprises one or more mutations relative to a wild-type and/or naturally occurring sequence of the gene encoding a lineage-specific cell-surface antigen. In some embodiments, the donor sequence comprises an artificial or heterologous sequence. In some embodiments, integration of the donor sequence produces a restriction nuclease site or a unique sequence tag in the gene encoding a lineage-specific cell-surface antigen of the genetically engineered hematopoietic cell. In some embodiments, integration of the donor sequence into the gene encoding a lineage-specific cell-surface antigen of the genetically engineered hematopoietic cell produces one or more silent mutations along with a non-silent mutation (e.g., one or more silent mutations along with alteration of the amino acid sequence of the epitope). In some embodiments, the one or more silent mutations are contiguous with another mutation described herein (e.g., contiguous with alteration of the amino acid sequence of the epitope). For example, in some embodiments, a genetically engineered hematopoietic cell comprises a genetic modification corresponding to alteration of the amino acid sequence of the epitope, e.g., a single nucleotide point mutation, and one or more silent mutations contiguous with the alteration (e.g., mutation). Accordingly, some aspects of the present disclosure provide a genetically engineered hematopoietic cell comprising a genetic modification corresponding to integration of a donor sequence as described herein, e.g., a donor sequence described herein.

It will be understood that, upon engrafting donor cells into a recipient host organism, the relative levels of the engrafted donor cells (and descendants thereof) and the host cells, e.g., in a given niche (e.g., bone marrow), are important for physiological and/or therapeutic outcomes for the host organism. The level of engrafted donor cells or descendants thereof relative to host cells in a given tissue or niche is referred to herein as “chimerism.” In some embodiments, a cell described herein (e.g., an HSC or HPC) is capable of engrafting in a human subject and does not exhibit any difference in chimerism as compared to a hematopoietic cell of the same cell type, but not comprising a genomic modification that results in expression of a variant form (e.g., comprising a mutated epitope) of a gene product. In some embodiments, a cell described herein (e.g., an HSC or HPC) capable of engrafting in a human subject exhibits no more than a 1%, no more than a 2%, no more than a 5%, no more than a 10%, no more than a 15%, no more than a 20%, no more than a 25%, no more than a 30%, no more than a 35%, no more than a 40%, no more than a 45%, or no more than a 50% difference in chimerism as compared to a hematopoietic cell of the same cell type, but not comprising a genomic modification that results in expression of a variant form (e.g., comprising a mutated epitope) of a gene product.

In some embodiments, a genetically engineered cell provided herein comprises only one genomic modification, e.g., a genomic modification that results in expression of a variant form (e.g., comprising a mutated epitope) of a gene product. In some embodiments, the genomic modification is a modification to a gene encoding a lineage-specific cell-surface antigen. It will be understood that the gene editing methods provided herein may result in genomic modifications in one or both alleles of a target gene. In some embodiments, genetically engineered cells comprising a genomic modification in both alleles of a given genetic locus are preferred.

In some embodiments, a genetically engineered cell provided herein comprises two or more genomic modifications, e.g., one or more genomic modifications in addition to a genomic modification that results in expression of a variant form (e.g., comprising a mutated epitope) of a gene product. For example, in some embodiments a genetically engineered cell comprises a modification to a gene encoding a lineage-specific cell-surface antigen and one or more additional genomic modifications, e.g., modification to a second gene or one or more silent mutations proximal to (e.g., contiguous with) the modification to the gene encoding a lineage-specific cell-surface antigen.

In some embodiments, a genetically engineered cell provided herein comprises a genomic modification that results in expression of a variant form (e.g., comprising a mutated epitope) of a gene encoding a lineage-specific cell-surface antigen. In some embodiments, the modification alters the amino acid sequence of an epitope that is recognized by an agent that specifically binds the lineage-specific cell-surface antigen. In some embodiments, the genomic modification does not substantially alter (e.g., impair, expand, or enhance) the function of the lineage-specific cell-surface antigen. In some embodiment, the modified lineage-specific cell-surface antigen has at least 50%, at least 60%, at least 70%, at least 80%, at least 90%, or at least 100% of the activity of a wild-type or a naturally occurring (i.e., unmodified) lineage-specific cell-surface antigen not comprising an altered epitope, such as in cells that are not subjected to the gene editing methods (e.g. HDR-mediated gene editing, base editing) described herein.

In some embodiments, the genomic modification does not substantially alter (e.g., increase or decrease) the expression of the lineage-specific cell-surface antigen. In some embodiments, the modified lineage-specific cell-surface antigen is expressed at a level that is at least 50%, at least 60%, at least 70%, at least 80%, at least 90%, or at least 100% of the level of expression of a wild-type or a naturally occurring (i.e., unmodified) lineage-specific cell-surface antigen not comprising an altered epitope, such as in cells that are not subjected to the gene editing methods (e.g. HDR-mediated gene editing, base editing) described herein.

In some embodiments, the genomic modification does not substantially alter (e.g., increase or decrease) the viability of a genetically engineered cell. In some embodiments, the genetically engineered cell has at least 50%, at least 60%, at least 70%, at least 80%, at least 90%, or at least 100% of the level of viability of a corresponding wild-type cell or of an otherwise similar cell not comprising the genomic modification, such as a cell that is not subjected to the gene editing methods (e.g., HDR-mediated gene editing, base editing) described herein.

In some embodiments, the immune effector cell is a lymphocyte. In some embodiments, the immune effector cell is a T-lymphocyte. In some embodiments, the T-lymphocyte is an alpha/beta T-lymphocyte. In some embodiments, the T-lymphocyte is a gamma/delta T-lymphocyte. In some embodiments, the immune effector cell is a natural killer T (NKT cell). In some embodiments, the immune effector cell is a natural killer (NK) cell. In some embodiments, the immune effector cell expresses a chimeric antigen receptor (CAR). In some embodiments, the immune effector cell does not express a CAR and/or does not express any transgenic protein except as provided by a genetic modification described herein (e.g., except as modified using a method using HDR or base editing described herein), e.g., except for a lineage-specific cell-surface antigen.

In some embodiments, the genetically engineered cells provided herein are hematopoietic cells, e.g., hematopoietic stem cells, hematopoietic progenitor cells (HPCs), hematopoietic stem or progenitor cells. Hematopoietic stem cells (HSCs) are cells characterized by pluripotency, self-renewal properties, and/or the ability to generate and/or reconstitute all lineages of the hematopoietic system, including both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSCs are characterized by the expression of one or more cell surface markers, e.g., CD34 (e.g., CD34+), which can be used for the identification and/or isolation of HSCs, and absence of cell surface markers associated with commitment to a cell lineage. In some embodiments, a genetically engineered cell (e.g., genetically engineered HSC) described herein does not express one or more cell-surface markers typically associated with HSC identification or isolation, expresses a reduced amount of the cell-surface markers, or expresses a variant cell-surface marker not recognized by an immunotherapeutic agent targeting the cell-surface marker, but nevertheless is capable of self-renewal and can generate and/or reconstitute all lineages of the hematopoietic system.

In some embodiments, a population of genetically engineered cells described herein comprises a plurality of genetically engineered hematopoietic stem cells. In some embodiments, a population of genetically engineered cells described herein comprises a plurality of genetically engineered hematopoietic progenitor cells. In some embodiments, a population of genetically engineered cells described herein comprises a plurality of genetically engineered hematopoietic stem cells and a plurality of genetically engineered hematopoietic progenitor cells.

In some embodiments, the genetically engineered HSCs are obtained from a subject, such as a human subject. Methods of obtaining HSCs are described, e.g., in International Publication No. WO 2017066760, which is herein incorporated by reference in its entirety. In some embodiments, the HSCs are peripheral blood HSCs. In some embodiments, the mammalian subject is a non-human primate, a rodent (e.g., mouse or rat), a bovine, a porcine, an equine, or a domestic animal. In some embodiments, the HSCs are obtained from a human subject, such as a human subject having a hematopoietic malignancy. In some embodiments, the HSCs are obtained from a healthy donor. In some embodiments, the HSCs are obtained from the subject to whom the immune cells expressing the chimeric antigen receptors will be subsequently administered. HSCs that are administered to the same subject from which the cells were obtained are referred to as autologous cells, whereas HSCs that are obtained from a subject who is not the subject to whom the cells will be administered are referred to as allogeneic cells.

In some embodiments, a population of genetically engineered cells is a heterogeneous population of cells, e.g., heterogeneous population of genetically engineered cells containing different mutations, e.g., different mutations in a gene encoding a lineage-specific cell-surface antigen. In some embodiments, at least 40%, at least 50%, at least 60%, at least 70%, at least 75%, at least 80%, at least 85%, at least 90%, or at least 95% of copies of a gene encoding a lineage-specific cell-surface antigen in the population of genetically engineered cells comprise a mutation effected by a genome editing approach described herein. By way of example, a population of genetically engineered cells can comprise a plurality of different mutations in a gene encoding a lineage-specific cell-surface antigen (e.g., a plurality of different mutations altering the amino acid sequence of an epitope of the lineage-specific cell-surface antigen) and each mutation of the plurality contributes to the percent of copies of the gene in the population of cells that have a mutation.

In some embodiments, the expression of a modified gene encoding a lineage-specific cell-surface antigen in the genetically engineered hematopoietic cell is compared to the expression of the unmodified gene in a reference hematopoietic cell (e.g., a wild-type counterpart, an otherwise similar hematopoietic cell not comprising the modification, or a mock genetically engineered hematopoietic cell (e.g., a hematopoietic cell that is contacted with Cas9 and a scrambled gRNA that does not effectively localize Cas9 or a base editor to the gene or a hematopoietic cell that is contacted with a targeting gRNA in the absence of Cas9 or the base editor).

In some embodiments, a cell (e.g., a hematopoietic cell, e.g., a hematopoietic stem cell) described herein is characterized by reduced binding or no binding of an agent that specifically binds to a lineage-specific cell-surface antigen. In some embodiments, a cell described herein comprises a modified lineage-specific cell-surface antigen which is not bound by an agent that specifically binds to the lineage-specific cell-surface antigen (i.e., the unmodified lineage-specific cell-surface antigen) or has reduced binding to an agent that specifically binds to the lineage-specific cell-surface antigen (i.e., the unmodified lineage-specific cell-surface antigen). In some embodiments, a cell is characterized by reduced binding of an agent that specifically binds to a lineage-specific cell-surface antigen relative to binding of the agent to a wildtype hematopoietic stem cell or an otherwise similar cell expressing not comprising the genomic modification (not comprising the modified lineage-specific cell-surface antigen). In some embodiments, cells having reduced or eliminated binding of an agent to a lineage-specific cell-surface antigen are resistant or immune to targeting by immunotherapeutic agents which specifically bind to the lineage-specific cell-surface antigen. In some embodiments, a genetically modified cell produced by a method described herein comprises a genetic modification that modifies an epitope of a lineage-specific cell-surface antigen and has reduced or eliminated binding of an agent that specifically binds to the lineage-specific cell-surface antigen relative to a wildtype cell or a cell not comprising the genomic modification. In some embodiments, the genetically modified cell can advantageously be administered to a subject to treat a cancer, autoimmune disease, or genetic disease and enable co-administration of an immunotherapeutic agent that might otherwise target the modified cell (e.g., and reduce its effectiveness). Lineage-specific cell surface antigens are known for a variety of cell types. In some embodiments, a lineage-specific cell-surface antigen is chosen from: BCMA, CD19, CD20, CD30, ROR1, B7H6, B7H3, CD23, CD33, CD38, C-type lectin like molecule-1, CS1, IL-5, L1-CAM, PSCA, PSMA, CD138, CD133, CD70, CD5, CD6, CD7, CD13, NKG2D, NKG2D ligand, CLEC12A, CD11, CD123, CD45, CD56, CD30, CD14, CD66b, CD41, CD61, CD62, CD235a, CD146, CD326, LMP2, CD22, CD52, CD10, CD3/TCR, CD79/BCR, EMR2 (CD312), and CD26. In some embodiments, a lineage-specific cell-surface antigen is chosen from: CD33, CD19, CD123, CLL-1, CD30, CD5, CD6, CD7, CD38, EMR2 (CD312), and BCMA. In some embodiments, a lineage-specific cell-surface antigen is chosen from: CD7, CD13, CD19, CD22, CD25, CD32, CD33, CD38, CD44, CD47, CD56, 96, CD117, CD123, CD135, CD174, CLL-1, folate receptor b, IL1RAP, MUC1, NKG2D/NKG2DL, TIM-3, and WT1. In some embodiments, a lineage-specific cell-surface antigen is chosen from: CD123, CD38, CD19, CD33, CD34, CD47, CLL-1, CD30, CD5, CD6, CD7, EMR2/CD312, and BCMA.

In some embodiments, a cell described herein comprises a genomic modification in a gene encoding a lineage-specific cell-surface antigen. In some embodiments, the lineage-specific cell-surface antigen is CD123, CD38, CD47, CD34, CD5, or CD19. In some embodiments, the lineage-specific cell-surface antigen is CD123 or CD38. In some embodiments, the lineage-specific cell-surface antigen is CD123. In some embodiments, the lineage-specific cell-surface antigen is CD38. In some embodiments, the lineage-specific cell-surface antigen is CD19. In some embodiments, the lineage-specific cell-surface antigen is CD34. In some embodiments, the lineage-specific cell-surface antigen is CD47. In some embodiments, the lineage-specific cell-surface antigen is CD5. In some embodiments, the lineage-specific cell-surface antigen is EMR2.

CD123 (also known as interleukin-3 receptor alpha or IL3Rα) is a type I cytokine receptor which binds to IL3. IL3 is a pleiotropic cytokine that regulates the function and production of hematopoietic and immune cells (see, e.g., Testa et al. Biomarker Research volume 2, Article number: 4 (2014)). Dysregulated expression of IL3 is associated with various cancers including myeloma (see, e.g., Lee et al. Blood (2004) 103 (6): 2308-2315). In some embodiments, a hematopoietic malignancy is characterized by cells expressing (e.g., over-expressing) CD123. Dysregulated expression of CD123 is associated with various hematopoietic malignancies including hairy cell leukemia, acute myeloid leukemia, blastic plasmacytoid dendritic cell neoplasm, and systemic mastocytosis (see, e.g., Del Giudice et al. Hematologica (2004) 89 (3): 303-308; Munoz et al. Hematologica (2001) 86 (12): 1261-1269; Angelot-Delettre et al. Hematologica (2015) 100 (2): 223-230; Alayed et al. American Journal of Hematology (2013) 88 (12): 1055-1061; Paradanani et al. Leukemia (2016) 30 (4): 914-918; Testa et al. Biomarker Research (2014) 2:4; and Lamble et al. Journal of Clinical Oncology (2022) 40 (3): 252-261). In some embodiments, CD123 is expressed by hematopoietic cells, e.g., hematopoietic stem cells and/or hematopoietic progenitor cells.

CD38 (also known as cyclic ADP ribose hydrolase) is a transmembrane ectoenzymatic glycoprotein involved in cell adhesion, signal transduction, and calcium signaling (see, e.g., van de Donk et al. Blood (2018) 131 (1): 13-29). In some embodiments, a hematopoietic malignancy is characterized by cells expressing (e.g., over-expressing) CD38. In some embodiments, CD38 is expressed by hematopoietic cells, e.g., hematopoietic stem cells and/or hematopoietic progenitor cells.

CD19 is a type I transmembrane glycoprotein comprising two extracellular Ig-like domains and a conserved C-terminal cytoplasmic tail that is typically expressed on the surface of human B cells and hematopoietic stem and progenitor cells committed to the B cell lineage. CD19 is required for B cell survival, development, and differentiation, and forms a multimolecular signaling complex on the surface of cells. CD19 has also been identified as a regulator of neoplastic growth and cell expansion in B cell cancers. The gene encoding human CD19 contains 7.41 kilobases and at least 15 exons, 4 of which encode extracellular domains; multiple alternatively spliced mRNA transcripts from the CD19 gene have been detected. In addition to its expression on B cells and B cell-committed hematopoietic cells, CD19 expression has also been associated with some hematopoietic malignancies.

EGF-like module-containing mucin-like hormone receptor-like 2 (EMR2), also referred to as CD312, is a 823-amino acid, ˜90 kDa protein (depending on isoform) of the EGF-seven-span transmembrane (TM7) family of adhesion G protein-coupled receptors (GPCR) with a high level of homology with CD97. EMR2 forms a heterodimer and binds to chondroitin sulfate B via its EGF-like domain 4 and mediate cell adhesion, granulocyte chemotaxis, degranulation, and the release of pro-inflammatory cytokines in macrophages. See, e.g. Kuan-Yu et al. Front. Immunol. (2017) 8:373. Without wishing to be bound by any particular theory, EMR2 is expressed on myeloid cells with highest expression in granulocytes, macrophages, and Kupffer cells. The ADGRE2 gene located on human chromosome 19 encodes human EMR2 and canonically contains 19 exons, although a number of isoforms exist with varying number EGF domains due to alternative RNA splicing. The dominant isoform in whole blood contains 17 exons. See, e.g. Safaee et al. Onc. Rev. (2014). 8 (242): 20-24.

CD5 is a member of the scavenger receptor cysteine-rich (SRCR) superfamily and functions as a signal transducing transmembrane glycoprotein involved in tyrosine phosphorylation on intracellular effector proteins. CD5 performs several functions in T- and B-lymphocyte receptor signaling and modulation of the immune system (see, e.g., Burgueño-Bucio et al. Journal of Leukocyte Biology (2019) 105 (5): 891-905). CD5 contains three SRCR domains which act as a receptor to regulate T-cell proliferation. CD5 is primarily expressed on thymocytes and mature T-lymphocytes. Additionally, CD5 expression in B-lymphocytes is associated with poor prognosis of large B-cell lymphoma (see, e.g., Tagawa et al. Cancer Research (2004) 64 (17): 5948-5955. The gene encoding human CD5 is located on chromosome 11 and contains 12 exons.

CD47 is a transmembrane integrin-associated protein belonging to the immunoglobulin superfamily and is involved in the increase of intracellular calcium concentration that occurs upon cell adhesion to extracellular matrix. CD47 binds to a variety of ligands including thrombospondin-1 and signal-regulatory protein alpha and functions in processes such as apoptosis, proliferation, adhesion, and migration. CD47 also has roles in immune and angiogenic responses including regulation of phagocytosis by macrophages (see, e.g., Brown and Frazier. Trends in Cell Biology (2001) 11 (3): 130-135). CD47 is widely expressed across various tissues in humans and also in solid tumors and hematological malignancies (see, e.g., Jiang et al. Journal of Hematology & Oncology (2021) 14:180). Human CD47 is located on chromosome 3 and contains 13 exons.

CD34 is a transmembrane phosphoglycoprotein belonging to the single-pass transmembrane sialomucin protein family that functions as a cell-cell adhesion factor. Accordingly, CD34 is an important adhesion molecule required for T-cells to enter lymph nodes and for attachment of hematopoietic stem cells to bone marrow extracellular matrix or to stromal cells. CD34 is highly expressed in hematopoietic stem and progenitor cells and endothelial cells. Moreover, CD34 is commonly found expressed on the cell surface of hematopoietic cancer cells (see, e.g., Sydney et al. Stem Cells (2014) 32 (6): 1380-1389; Nielsen and McNagny. Journal of Cell Science (2008) 121 (22): 3683-3692; Lanze et al. Journal of Biological Regulators and Homeostatic Agents (2001) 15 (1): 1-13; Sutherland and Keating. Journal of Hematotherapy (2009) 1 (2): 115-129). CD34 is located on chromosome 1 and contains 8 exons.

Due to the shared expression of CD33, CD123, CD19, CLL-1, CD30, CD5, CD6, CD7, CD34, CD38, CD47, EMR2/CD312, and/or BCMA on both normal, healthy cells (e.g., healthy hematopoietic cells) as well as being an expressed antigen on malignant cells, therapeutic targeting of CD33, CD123, CD19, CLL-1, CD30, CD5, CD6, CD7, CD34, CD38, CD47, EMR2/CD312, and/or BCMA can result in depletion of healthy hematopoietic cell and/or progenitor cell pools.

In some embodiments, a cell described herein comprises a genomic modification that results in a mutation of a gene encoding a lineage-specific cell surface antigen. In some embodiments, the mutation of a gene encoding a lineage-specific cell-surface antigen alters one or more amino acids of the lineage-specific cell-surface antigen. In some embodiments, the one or more amino acids are part of an epitope recognized (i.e., bound by) an agent that specifically binds to the lineage-specific cell-surface antigen. In some embodiments, the epitope is part of a domain, e.g., the extracellular domain or a sub-domain thereof, of the lineage-specific cell-surface antigen.

Alterations of one or more amino acids may comprise one, two, or all of substitution, insertion, or deletion. For example, an alteration may comprise substitution of amino acids recited herein with different amino acids. As a further example, an alteration may comprise deletion of amino acids recited herein. As a further example, an alteration may comprise insertion of one or more amino acids at a position recited herein or as part of a deletion of amino acids recited herein.

In some embodiments, a mutation of a gene encoding CD123 alters one or more amino acids associated with an epitope of CD123. In some embodiments, the epitope of CD123 is a portion of CD123 bound by an agent, e.g., an immunotherapeutic agent. In some embodiments, the agent is an anti-CD123 antibody. In some embodiments, the agent comprises an anti-CD123 antibody or portion thereof, e.g., an antibody drug conjugate (ADC), a chimeric antigen receptor (CAR), or a multispecific antibody (e.g., a bispecific T cell engager). For example, the agent can be anti-CD123 antibody 7G3 or a variant thereof (e.g., a humanized variant, e.g., antibody CSL-36). In some embodiments, the agent is an anti-CD123 drug, e.g., talacotuzumab. In some embodiments, the epitope of CD123 is one or more amino acids of a protein domain (e.g., the extracellular domain) or the amino acids encoded by an exon or combination of exons of the gene encoding CD123. In some embodiments, the epitope of CD123 comprises one or more amino acids encoded by exon 3 of the gene encoding CD123. In some embodiments, the epitope of CD123 comprises one or more amino acids encoded by exon 4 of the gene encoding CD123. In some embodiments, the epitope of CD123 comprises one or more (e.g., two or more, three or more, four or more, or all) of the amino acids at positions 51, 59, 61, 82, or 84 of a wildtype gene encoding CD123 or at corresponding positions in a homologous CD123 gene.

In some embodiments, a mutation of a gene encoding CD123 comprises a substitution of the amino acid at position 51 of a wildtype CD123 or at a corresponding position in a homologous CD123 gene. In some embodiments, a lysine is substituted for the amino acid at position 51 of a wildtype CD123 or at a corresponding position in a homologous CD123 gene. In some embodiments, a glycine is substituted for the amino acid at position 51 of a wildtype CD123 or at a corresponding position in a homologous CD123 gene. In some embodiments, a mutation of a gene encoding CD123 comprises a substitution of the amino acid at position 59 of a wildtype CD123 or at a corresponding position in a homologous CD123 gene. In some embodiments, a phenylalanine is substituted for the amino acid at position 59 of a wildtype CD123 or at a corresponding position in a homologous CD123 gene. In some embodiments, a cysteine is substituted for the amino acid at position 59 of a wildtype CD123 or at a corresponding position in a homologous CD123 gene. In some embodiments, a mutation of a gene encoding CD123 comprises a substitution of the amino acid at position 61 of a wildtype CD123 or at a corresponding position in a homologous CD123 gene. In some embodiments, a leucine is substituted for the amino acid at position 61 of a wildtype CD123 or at a corresponding position in a homologous CD123 gene. In some embodiments, a mutation of a gene encoding CD123 comprises a substitution of the amino acid at position 82 of a wildtype CD123 or at a corresponding position in a homologous CD123 gene. In some embodiments, an alanine is substituted for the amino acid at position 82 of a wildtype CD123 or at a corresponding position in a homologous CD123 gene. In some embodiments, a mutation of a gene encoding CD123 comprises a substitution of the amino acid at position 84 of a wildtype CD123 or at a corresponding position in a homologous CD123 gene. In some embodiments, a glutamine is substituted for the amino acid at position 84 of a wildtype CD123 or at a corresponding position in a homologous CD123 gene. In some embodiments, an alanine is substituted for the amino acid at position 84 of a wildtype CD123 or at a corresponding position in a homologous CD123 gene.

In some embodiments, a mutation of a gene encoding CD123 makes a change in the amino acid sequence corresponding to the amino acid sequence of a CD123 ortholog. In some embodiments, a mutation substitutes an amino acid of human CD123 for an amino acid at a corresponding position of an orthologous CD123, e.g., a non-human primate CD123. In some embodiments, a mutation inserts or deletes one or more amino acids of human CD123 to correspond to the sequence of an orthologous CD123, e.g., a non-human primate CD123. In some embodiments, a mutation changes the amino acid sequence in a manner corresponding to a tolerable genetic variant identified by one or more genomic sequence comparison algorithms, e.g., gnomAD (see, e.g., Gudmundsson et al. arXiv: 2107.11458v3, e.g., gnomad.broadinstitute.org/) or to a position characterized by a plurality of tolerable genetic variants. In some embodiments, mutations to CD123 corresponding to the amino acid sequence of a CD123 ortholog or at positions characterized by a plurality of tolerable genetic variants decrease or eliminate binding of an immunotherapeutic agent targeting CD123 while preserving some or all of CD123 structure, expression, and/or functionality, providing a cell expressing CD123 (e.g., functional CD123) that is targeted less or not at all by anti-CD123 immunotherapeutic agents. In some embodiments, alteration results in a missense variant of CD123.

In some embodiments, a mutation of a gene encoding CD38 alters one or more amino acids associated with an epitope of CD38. In some embodiments, the epitope of CD38 is a portion of CD38 bound by an agent, e.g., an immunotherapeutic agent. In some embodiments, the agent is an anti-CD38 antibody. In some embodiments, the agent comprises an anti-CD38 antibody or portion thereof, e.g., an antibody drug conjugate (ADC), a chimeric antigen receptor (CAR), or a multispecific antibody (e.g., a bispecific T cell engager). For example, the agent can be anti-CD38 antibody HB7 or a variant thereof (e.g., a humanized variant). In some embodiments, the agent is an anti-CD38 drug, e.g., daratumumab. In some embodiments, the epitope of CD38 is one or more amino acids of a protein domain (e.g., the extracellular domain) or the amino acids encoded by an exon or combination of exons of the gene encoding CD38. In some embodiments, the epitope of CD38 comprises one or more amino acids encoded by exon 7 of the gene encoding CD38. In some embodiments, the epitope of CD38 comprises one or more (e.g., two or more, three or more, four or more, or all) of the amino acids at positions 270-274 of a wildtype gene encoding CD38 or at corresponding positions in a homologous CD38 gene.

In some embodiments, a mutation of a gene encoding CD38 comprises a substitution of the amino acid at position 270 of a wildtype CD38 or at a corresponding position in a homologous CD38 gene. In some embodiments, an alanine is substituted for the amino acid at position 270 of a wildtype CD38 or at a corresponding position in a homologous CD38 gene. In some embodiments, a mutation of a gene encoding CD38 comprises a substitution of the amino acid at position 271 of a wildtype CD38 or at a corresponding position in a homologous CD38 gene. In some embodiments, a mutation of a gene encoding CD38 comprises a substitution of the amino acid at position 272 of a wildtype CD38 or at a corresponding position in a homologous CD38 gene. In some embodiments, a histidine is substituted for the amino acid at position 272 of a wildtype CD38 or at a corresponding position in a homologous CD38 gene. In some embodiments, an arginine is substituted for the amino acid at position 272 of a wildtype CD38 or at a corresponding position in a homologous CD38 gene. In some embodiments, an alanine is substituted for the amino acid at position 272 of a wildtype CD38 or at a corresponding position in a homologous CD38 gene. In some embodiments, a mutation of a gene encoding CD38 comprises a substitution of the amino acid at position 273 of a wildtype CD38 or at a corresponding position in a homologous CD38 gene. In some embodiments, a mutation of a gene encoding CD38 comprises a substitution of the amino acid at position 274 of a wildtype CD38 or at a corresponding position in a homologous CD38 gene. In some embodiments, a phenylalanine is substituted for the amino acid at position 274 of a wildtype CD38 or at a corresponding position in a homologous CD38 gene.

In some embodiments, a mutation of a gene encoding CD38 makes a change in the amino acid sequence corresponding to the amino acid sequence of a CD38 ortholog. In some embodiments, a mutation substitutes an amino acid of human CD38 for an amino acid at a corresponding position of an orthologous CD38, e.g., a non-human primate CD38. In some embodiments, a mutation inserts or deletes one or more amino acids of human CD38 to correspond to the sequence of an orthologous CD38, e.g., a non-human primate CD38. In some embodiments, a mutation changes the amino acid sequence in a manner corresponding to a tolerable genetic variant identified by one or more genomic sequence comparison algorithms, e.g., gnomAD, or to a position characterized by a plurality of tolerable genetic variants. In some embodiments, mutations to CD38 corresponding to the amino acid sequence of a CD38 ortholog or at positions characterized by a plurality of tolerable genetic variants decrease or eliminate binding of an immunotherapeutic agent targeting CD38 while preserving some or all of CD38 structure, expression, and/or functionality, providing a cell expressing CD38 (e.g., functional CD38) that is targeted less or not at all by anti-CD38 immunotherapeutic agents.

In some embodiments, a mutation of a gene encoding CD19 alters one or more amino acids associated with an epitope of CD19. In some embodiments, the epitope of CD19 is a portion of CD19 bound by an agent, e.g., an immunotherapeutic agent. In some embodiments, the agent is an anti-CD19 antibody. In some embodiments, the agent is the anti-CD19 antibody FMC63 or HIB19. In some embodiments, the agent comprises an anti-CD19 antibody or portion thereof, e.g., an antibody drug conjugate (ADC), a chimeric antigen receptor (CAR), or a multispecific antibody (e.g., a bispecific T cell engager). In some embodiments, the agent is an anti-CD19 drug. In some embodiments, the epitope of CD19 corresponds to the amino acids of a protein domain (e.g., the extracellular first or second Ig-like domains or non-Ig like domain) or the amino acids encoded by an exon or combination of exons of the gene encoding CD19. In some embodiments, the epitope of CD19 comprises the amino acids encoded by one, two, three, or all of exons 1, 2, 3, or 4 of CD19. In some embodiments, the epitope of CD19 comprises the amino acids encoded by exon 2 of CD19. In some embodiments, the epitope of CD19 comprises the amino acids encoded by exon 4 of CD19. In some embodiments, the CD19 epitope comprises amino acids 216-238, 216-236, 216-234, 216-232, 216-230, 216-228, 216-226, 216-224, 216-222, 216-220, 216-218, 218-238, 218-236, 218-234, 218-232, 218-230, 218-228, 218-226, 218-224, 218-222, 218-220, 220-238, 220-236, 220-234, 220-232, 220-230, 220-228, 220-226, 220-224, 220-222, 222-238, 222-236, 222-234, 222-232, 222-230, 222-228, 222-226, 222-224, 224-238, 224-236, 224-234, 224-232, 224-230, 224-228, 224-226, 226-238, 226-236, 226-234, 226-232, 226-230, 226-228, 228-238, 228-236, 228-234, 228-232, 228-230, 230-238, 230-236, 230-234, 230-232, 232-238, 232-236, 232-234, 234-238, 234-236, or 236-238 of CD19, e.g., 216-224 or 218-238 of CD19. In some embodiments, the CD19 epitope comprises amino acid 163 and/or 164 of CD19.

In some embodiments, a mutation of a gene encoding CD19 makes a change in the amino acid sequence corresponding to the amino acid sequence of a CD19 ortholog. In some embodiments, an alteration comprises substitution of amino acid at position 218 of a wildtype CD19 or at a corresponding position in a homologous CD19. In some embodiments, an alteration comprises insertion of one or more amino acids at position 224 of a wildtype CD19 or at a corresponding position in a homologous CD19. In some embodiments, an alteration comprises substitution of amino acid 218 of CD19 and insertion of one or more amino acids at position 224 of a wildtype CD19 or at corresponding positions in a homologous CD19.

In some embodiments, an alteration comprises substitution of amino acid 163 and/or 164 of a wildtype CD19 or at a corresponding position in a homologous CD38. In some embodiments, an alteration comprises substitution of amino acid 163 of a wildtype CD19 or at a corresponding position in a homologous CD38. In some embodiments, an alteration comprises substitution of amino acid 164 of a wildtype CD19 or at a corresponding position in a homologous CD38. In some embodiments, an alteration comprises substitution of amino acids 163 and 164 of a wildtype CD19 or at a corresponding position in a homologous CD38. In some embodiments, a leucine is substituted for the amino acid at position 163 of a wildtype CD19 or at a corresponding position in a homologous CD19. In some embodiments, a cysteine is substituted for the amino acid at position 163 of a wildtype CD19 or at a corresponding position in a homologous CD19. In some embodiments, a cystine is substituted for the amino acid at position 163 and a phenylalanine is substituted for the amino acid at position 164 of a wildtype CD19 or a corresponding position in a homologous CD19.

In some embodiments, alteration results in a missense variant of CD19. In some embodiments, alteration results in a change at a splice region in CD19.

In some embodiments, a mutation of a gene encoding CD19 makes a change in the amino acid sequence corresponding to the amino acid sequence of a CD19 ortholog. In some embodiments, a mutation substitutes an amino acid of human CD19 for an amino acid at a corresponding position of an orthologous CD19, e.g., a non-human primate CD19. In some embodiments, a mutation inserts or deletes one or more amino acids of human CD19 to correspond to the sequence of an orthologous CD19, e.g., a non-human primate CD19. For example, in some embodiments, histidine 218 is replaced with arginine, corresponding to the rhesus CD19 sequence at that position. As a further example, in some embodiments, an amino acid (e.g., serine) is inserted at position 224 of human CD19, corresponding to the rhesus CD19 sequence at that position. In some embodiments, mutations to CD19 corresponding to the amino acid sequence of a CD19 ortholog decrease or eliminate binding of an immunotherapeutic agent targeting CD19 while preserving some or all of CD19 expression and/or functionality, providing a cell expressing CD19 (e.g., functional CD19) that is targeted less or not at all by anti-CD19 immunotherapeutic agents.

In some embodiments, a mutation of a gene encoding EMR2 alters one or more amino acids associated with an epitope of EMR2. In some embodiments, the epitope of EMR2 is a portion of EMR2 bound by an agent, e.g., an immunotherapeutic agent. In some embodiments, the agent is an anti-EMR2 antibody. In some embodiments, the agent comprises an anti-EMR2 antibody or portion thereof, e.g., an antibody drug conjugate (ADC), a chimeric antigen receptor (CAR), or a multispecific antibody (e.g., a bispecific T cell engager). In some embodiments, the agent is an anti-EMR2 drug. In some embodiments, the epitope of EMR2 corresponds to the amino acids of a protein domain (e.g., the extracellular domain) or the amino acids encoded by an exon or combination of exons of the gene encoding EMR2. In some embodiments, the epitope of EMR2 comprises amino acids encoded by one, two, three, four, or all of exons 6, 10, 11, 14, and 18 of EMR2. In some embodiments, the epitope of EMR2 comprises the amino acids encoded by exon 6 of EMR2. In some embodiments, the epitope of EMR2 comprises the amino acids encoded by exon 10 of EMR2. In some embodiments, the epitope of EMR2 comprises the amino acids encoded by exon 11 of EMR2. In some embodiments, the epitope of EMR2 comprises the amino acids encoded by exon 14 of EMR2. In some embodiments, the epitope of EMR2 comprises the amino acids encoded by exon 18 of EMR2.

In some embodiments, a mutation of a gene encoding EMR2 makes a change in the amino acid sequence corresponding to the amino acid sequence of a EMR2 ortholog. In some embodiments, an alteration comprises substitution of amino acid at any one or more of positions 124, 132, 146, 292, 294, 295, 296, 298, 299, 303, 304, 305, 306, 307, 308, 312, 318, 320, 328, 329, 331, 332, 335, 340, 347, 527, or 708 of a wildtype EMR2 or at a corresponding position in a homologous EMR2.

In some embodiments, alteration results in a missense variant of EMR2. In some embodiments, alteration results in a change at a splice region in EMR2.

In some embodiments, a mutation of a gene encoding EMR2 alters one or more amino acids associated with an epitope of EMR2. In some embodiments, the epitope of EMR2 is a portion of EMR2 bound by an agent, e.g., an immunotherapeutic agent. In some embodiments, the agent is an anti-EMR2 antibody. In some embodiments, the agent comprises an anti-EMR2 antibody or portion thereof, e.g., an antibody drug conjugate (ADC), a chimeric antigen receptor (CAR), or a multispecific antibody (e.g., a bispecific T cell engager). For example, the agent can be anti-EMR2 monoclonal antibody 2A1 (Thermo Fisher) or a variant thereof (e.g., a humanized variant), Q9UHX3, OASA01861, AB 2738756, NLS6381, ab75190, MAB4894, A100,000. Additional anti-EMR2 antibodies will be evident to one of ordinary skill in the art. See, e.g., International Publication No. WO 2017/087800 A1; Chang et al. FEBS Letters. (2003) 547 (1-3): 145-150; Yona et al. FASEB J. (2008). 22 (3): 741-751.

In some embodiments, mutations to EMR2 corresponding to the amino acid sequence of a EMR2 ortholog decrease or eliminate binding of an immunotherapeutic agent targeting EMR2 while preserving some or all of EMR2 expression and/or functionality, providing a cell expressing EMR2 (e.g., functional EMR2) that is targeted less or not at all by anti-EMR2 immunotherapeutic agents.

In some embodiments, a mutation of a gene encoding CD5 alters one or more amino acids associated with an epitope of CD5. In some embodiments, the epitope of CD5 is a portion of CD5 bound by an agent, e.g., an immunotherapeutic agent. In some embodiments, the agent is an anti-CD5 antibody. In some embodiments, the agent comprises an anti-CD5 antibody or portion thereof, e.g., an antibody drug conjugate (ADC), a chimeric antigen receptor (CAR), or a multispecific antibody (e.g., a bispecific T cell engager). For example, the agent can be anti-CD5 monoclonal antibody H65 or a variant thereof (e.g., a humanized variant). In some embodiments, the agent is an anti-CD5 drug (e.g., Zolimomab). In some embodiments, the epitope of CD5 is one or more amino acids of a protein domain (e.g., the extracellular domain) or the amino acids encoded by an exon or combination of exons of the gene encoding CD5.

In some embodiments, the epitope of CD5 comprises one or more (e.g., two or more, three or more, four or more, or all) of the amino acids 35-133 of a wildtype gene encoding CD5 or at corresponding positions in a homologous CD5 gene.

In some embodiments, the modification of an epitope of CD5 comprises an insertion, deletion, substitution, or inversion of one or more amino acids (e.g., one, two, three, four or more) occurring at positions 35-133 of a wildtype CD5. In some embodiments, alteration results in a missense variant of CD5 occurring at one or more (e.g., one, two, three, four or more) amino acids occurring at positions 35-133 of a wildtype CD5.

In some embodiments, alteration results in a missense variant of CD5. In some embodiments, alteration results in a change at a splice region in CD5.

In some embodiments, a mutation of a gene encoding CD5 makes a change in the amino acid sequence corresponding to the amino acid sequence of a CD5 ortholog. In some embodiments, a mutation substitutes an amino acid of human CD5 for an amino acid at a corresponding position of an orthologous CD5, e.g., a non-human primate CD5. In some embodiments, a mutation inserts or deletes one or more amino acids of human CD5 to correspond to the sequence of an orthologous CD5, e.g., a non-human primate CD5. In some embodiments, a mutation changes the amino acid sequence in a manner corresponding to a tolerable genetic variant identified by one or more genomic sequence comparison algorithms, e.g., gnomAD (see, e.g., Gudmundsson et al. arXiv: 2107.11458v3, e.g., gnomad.broadinstitute.org/), or to a position characterized by a plurality of tolerable genetic variants.

In some embodiments, mutations to CD5 corresponding to the amino acid sequence of a CD5 ortholog or at positions characterized by a plurality of tolerable genetic variants decrease or eliminate binding of an immunotherapeutic agent targeting CD5 while preserving some or all of CD5 structure, expression, and/or functionality, providing a cell expressing CD5 (e.g., functional CD5) that is targeted less or not at all by anti-CD5 immunotherapeutic agents.

In some embodiments, a mutation of a gene encoding CD47 alters one or more amino acids associated with an epitope of CD47. In some embodiments, the epitope of CD47 is a portion of CD47 bound by an agent, e.g., an immunotherapeutic agent. In some embodiments, the agent is an anti-CD47 antibody. In some embodiments, the agent is the anti-CD47 B6H12 or 2D3 antibody. In some embodiments, the agent comprises an anti-CD47 antibody or portion thereof, e.g., an antibody drug conjugate (ADC), a chimeric antigen receptor (CAR), or a multispecific antibody (e.g., a bispecific T cell engager). For example, the agent can be anti-CD47 antibody or a variant thereof (e.g., a humanized variant). In some embodiments, the agent is an anti-CD47 drug. In some embodiments, the epitope of CD47 is one or more amino acids of a protein domain (e.g., the extracellular domain) or the amino acids encoded by an exon or combination of exons of the gene encoding CD47.

In some embodiments, the epitope of CD47 comprises one or more of amino acids 117-122 in CD47. In some embodiments, one or more of amino acids 117-122 in CD47 is deleted. In some embodiments, amino acids 117-122 in CD47 are deleted. In some embodiments, amino acids 117, 118, 119, 120, 121, and/or 122 or any combination thereof in CD47 is deleted. In some embodiments, the epitope of CD47 comprises one or more of amino acids 52-55 in CD47. In some embodiments, one or more of amino acids 52-55 in CD47 is deleted. In some embodiments, amino acids 52-55 in CD47 are deleted. In some embodiments, amino acids 52, 53, 54, and/or 55 or any combination thereof in CD47 is deleted.

In some embodiments, a mutation of a gene encoding CD47 comprises a substitution of the amino acid at position 31 of a wildtype CD47 or at a corresponding position in a homologous CD47 gene. In some embodiments, a methionine is substituted for the amino acid at position 31 of a wildtype CD47 or at a corresponding position in a homologous CD47 gene. In some embodiments, a mutation of a gene encoding CD47 comprises a substitution of the amino acid at position 47 of a wildtype CD47 or at a corresponding position in a homologous CD47 gene. In some embodiments, a histidine is substituted for the amino acid at position 47 of a wildtype CD47 or at a corresponding position in a homologous CD47 gene. In some embodiments, a glycine is substituted for the amino acid at position 47 of a wildtype CD47 or at a corresponding position in a homologous CD47 gene. In some embodiments, a mutation of a gene encoding CD47 comprises a substitution of the amino acid at position 49 of a wildtype CD47 or at a corresponding position in a homologous CD47 gene. In some embodiments, an arginine is substituted for the amino acid at position 49 of a wildtype CD47 or at a corresponding position in a homologous CD47 gene. In some embodiments, a proline is substituted for the amino acid at position 49 of a wildtype CD47 or at a corresponding position in a homologous CD47 gene. In some embodiments, a mutation of a gene encoding CD47 comprises a substitution of the amino acid at position 52 of a wildtype CD47 or at a corresponding position in a homologous CD47 gene. In some embodiments, a mutation of a gene encoding CD47 comprises a deletion of the amino acid at position 52 of a wildtype CD47 or at a corresponding position in a homologous CD47 gene. In some embodiments, a mutation of a gene encoding CD47 comprises a substitution of the amino acid at position 53 of a wildtype CD47 or at a corresponding position in a homologous CD47 gene. In some embodiments, an alanine is substituted for the amino acid at position 53 of a wildtype CD47 or at a corresponding position in a homologous CD47 gene. In some embodiments, a mutation of a gene encoding CD47 comprises a deletion of the amino acid at position 53 of a wildtype CD47 or at a corresponding position in a homologous CD47 gene. In some embodiments, a mutation of a gene encoding CD47 comprises a deletion of the amino acid at position 54 of a wildtype CD47 or at a corresponding position in a homologous CD47 gene. In some embodiments, a mutation of a gene encoding CD47 comprises a deletion of the amino acid at position 55 of a wildtype CD47 or at a corresponding position in a homologous CD47 gene. In some embodiments, a mutation of a gene encoding CD47 comprises a substitution of the amino acid at position 120 of a wildtype CD47 or at a corresponding position in a homologous CD47 gene. In some embodiments, an alanine is substituted for the amino acid at position 120 of a wildtype CD47 or at a corresponding position in a homologous CD47 gene. In some embodiments, a mutation of a gene encoding CD47 comprises a substitution of the amino acid at position 124 of a wildtype CD47 or at a corresponding position in a homologous CD47 gene. In some embodiments, a lysine is substituted for the amino acid at position 124 of a wildtype CD47 or at a corresponding position in a homologous CD47.

In some embodiments, a mutation of a gene encoding CD47 makes a change in the amino acid sequence corresponding to the amino acid sequence of a CD47 ortholog. In some embodiments, a mutation substitutes an amino acid of human CD47 for an amino acid at a corresponding position of an orthologous CD47, e.g., a non-human primate CD47. In some embodiments, a mutation inserts or deletes one or more amino acids of human CD47 to correspond to the sequence of an orthologous CD47, e.g., a non-human primate CD47. In some embodiments, a mutation changes the amino acid sequence in a manner corresponding to a tolerable genetic variant identified by one or more genomic sequence comparison algorithms, e.g., gnomAD (see, e.g., Gudmundsson et al. arXiv: 2107.11458v3, e.g., gnomad.broadinstitute.org/), or to a position characterized by a plurality of tolerable genetic variants.

In some embodiments, mutations to CD47 corresponding to the amino acid sequence of a CD47 ortholog or at positions characterized by a plurality of tolerable genetic variants decrease or eliminate binding of an immunotherapeutic agent targeting CD47 while preserving some or all of CD47 structure, expression, and/or functionality, providing a cell expressing CD47 (e.g., functional CD47) that is targeted less or not at all by anti-CD47 immunotherapeutic agents.

In some embodiments, a mutation of a gene encoding CD34 alters one or more amino acids associated with an epitope of CD34. In some embodiments, the epitope of CD34 is a portion of CD34 bound by an agent, e.g., an immunotherapeutic agent. In some embodiments, the agent is an anti-CD34 antibody. In some embodiments, the anti-CD34 antibody is clone QBend10 or 561. In some embodiments, the agent comprises an anti-CD34 antibody or portion thereof, e.g., an antibody drug conjugate (ADC), a chimeric antigen receptor (CAR), or a multispecific antibody (e.g., a bispecific T cell engager). In some embodiments, the epitope of CD34 is one or more amino acids of a protein domain (e.g., the extracellular domain) or the amino acids encoded by an exon or combination of exons of the gene encoding CD34.

In some embodiments, a mutation of a gene encoding CD34 comprises a substitution of the amino acid at position 42 of a wildtype CD34 or at a corresponding position in a homologous CD34 gene. In some embodiments, a glycine is substituted for the amino acid at position 42 of a wildtype CD34 or at a corresponding position in a homologous CD34 gene. In some embodiments, a mutation of a gene encoding CD34 comprises a substitution of the amino acid at position 46 of a wildtype CD34 or at a corresponding position in a homologous CD34 gene. In some embodiments, an alanine is substituted for the amino acid at position 46 of a wildtype CD34 or at a corresponding position in a homologous CD34 gene. In some embodiments, a mutation of a gene encoding CD34 comprises a substitution of the amino acid at position 47 of a wildtype CD34 or at a corresponding position in a homologous CD34 gene. In some embodiments, a lysine is substituted for the amino acid at position 47 of a wildtype CD34 or at a corresponding position in a homologous CD34 gene. In some embodiments, a glutamate is substituted for the amino acid at position 47 of a wildtype CD34 or at a corresponding position in a homologous CD34 gene. In some embodiments, a mutation of a gene encoding CD34 comprises a substitution of the amino acid at position 49 of a wildtype CD34 or at a corresponding position in a homologous CD34 gene. In some embodiments, a proline is substituted for the amino acid at position 49 of a wildtype CD34 or at a corresponding position in a homologous CD34 gene. In some embodiments, a serine is substituted for the amino acid at position 49 of a wildtype CD34 or at a corresponding position in a homologous CD34 gene. In some embodiments, a mutation of a gene encoding CD34 comprises a substitution of the amino acid at position 50 of a wildtype CD34 or at a corresponding position in a homologous CD34 gene. In some embodiments, an alanine is substituted for the amino acid at position 50 of a wildtype CD34 or at a corresponding position in a homologous CD34 gene. In some embodiments, a proline is substituted for the amino acid at position 50 of a wildtype CD34 or at a corresponding position in a homologous CD34 gene. In some embodiments, a mutation of a gene encoding CD34 comprises a substitution of the amino acid at position 51 of a wildtype CD34 or at a corresponding position in a homologous CD34 gene. In some embodiments, an alanine is substituted for the amino acid at position 51 of a wildtype CD34 or at a corresponding position in a homologous CD34 gene. In some embodiments, a mutation of a gene encoding CD34 comprises a substitution of the amino acid at position 54 of a wildtype CD34 or at a corresponding position in a homologous CD34 gene. In some embodiments, an alanine is substituted for the amino acid at position 54 of a wildtype CD34 or at a corresponding position in a homologous CD34 gene. In some embodiments, a mutation of a gene encoding CD34 comprises a substitution of the amino acid at position 55 of a wildtype CD34 or at a corresponding position in a homologous CD34 gene. In some embodiments, an alanine is substituted for the amino acid at position 55 of a wildtype CD34 or at a corresponding position in a homologous CD34 gene.

In some embodiments, a mutation of a gene encoding CD34 makes a change in the amino acid sequence corresponding to the amino acid sequence of a CD34 ortholog. In some embodiments, a mutation substitutes an amino acid of human CD34 for an amino acid at a corresponding position of an orthologous CD34, e.g., a non-human primate CD34. In some embodiments, a mutation inserts or deletes one or more amino acids of human CD34 to correspond to the sequence of an orthologous CD34, e.g., a non-human primate CD34. In some embodiments, a mutation changes the amino acid sequence in a manner corresponding to a tolerable genetic variant identified by one or more genomic sequence comparison algorithms, e.g., gnomAD (see, e.g., Gudmundsson et al. arXiv: 2107.11458v3, e.g., gnomad.broadinstitute.org/), or to a position characterized by a plurality of tolerable genetic variants. In some embodiments, mutations to CD34 corresponding to the amino acid sequence of a CD34 ortholog or at positions characterized by a plurality of tolerable genetic variants decrease or eliminate binding of an immunotherapeutic agent targeting CD34 while preserving some or all of CD34 structure, expression, and/or functionality, providing a cell expressing CD34 (e.g., functional CD34) that is targeted less or not at all by anti-CD34 immunotherapeutic agents.

### Methods of Editing Cells

Some aspects of this disclosure provide compositions and methods for generating the genetically engineered cells described herein, e.g., genetically engineered cells comprising a modification in their genome that results in alteration of the amino acid sequence of an epitope of a lineage-specific cell-surface antigen, or expression of a variant form of the lineage-specific cell-surface antigen that is not recognized by an agent (e.g., an immunotherapeutic agent) targeting (e.g., that specifically binds) the lineage-specific cell-surface antigen. Such compositions and methods provided herein include, without limitation, suitable strategies and approaches for genetically engineering cells, e.g., by using RNA-guided nucleases, such as CRISPR/Cas nucleases including base editors, and suitable RNAs able to bind such RNA-guided nucleases and target them to a suitable target site within the genome of a cell to effect a genomic modification resulting in alteration of the amino acid sequence of an epitope of a lineage-specific cell-surface antigen, or expression of a variant form of the lineage-specific cell-surface antigen that is not recognized by an immunotherapeutic agent targeting the lineage-specific cell-surface antigen.

In some embodiments, a genetically engineered cell (e.g., a genetically engineered hematopoietic cell, such as, for example, a genetically engineered hematopoietic stem or progenitor cell or a genetically engineered immune effector cell (e.g., a B cell or T cell)) described herein is generated via genome editing technology, which includes any technology capable of introducing targeted changes, also referred to as “edits,” into the genome of a cell.

One exemplary suitable genome editing technology is “gene editing,” comprising the use of a RNA-guided nuclease, e.g., a CRISPR/Cas nuclease, to introduce targeted single- or double-stranded DNA breaks in the genome of a cell, which trigger cellular repair mechanisms, such as, for example, nonhomologous end joining (NHEJ), microhomology-mediated end joining (MMEJ, also sometimes referred to as “alternative NHEJ” or “alt-NHEJ”), or homology-directed repair (HDR) that typically result in an altered nucleic acid sequence (e.g., via nucleotide or nucleotide sequence insertion, deletion, inversion, or substitution) at or immediately proximal to the site of the nuclease cut. See, Yeh et al. Nat. Cell. Biol. (2019) 21:1468-1478; e.g., Hsu et al. Cell (2014) 157:1262-1278; Jasin et al. DNA Repair (2016) 44:6-16; Sfeir et al. Trends Biochem. Sci. (2015) 40:701-714. In some embodiments, a genomic modification is introduced into a cell using HDR, e.g., as described herein.

Another exemplary suitable genome editing technology is “base editing,” which includes the use of a base editor, e.g., a nuclease-impaired or partially nuclease-impaired RNA-guided CRISPR/Cas protein fused to a deaminase that targets and deaminates a specific nucleobase, e.g., a cytosine or adenosine nucleobase of a C or A nucleotide, which, via cellular mismatch repair mechanisms, results in a change from a C to a T nucleotide (also changing a G to an A nucleotide on the opposite strand), or a change from an A to a G nucleotide (also inducing a G to a C nucleotide on the opposite strand). Base editors or “BEs” that catalyze conversion of a C to a T nucleotide may be referred to as a “cytosine base editor” or “CBE,” while base editors that catalyze conversion of an A to a G nucleotide may be referred to as an “adenosine base editor” or “ABE.” See, e.g., Komor et al. Nature (2016) 533:420-424; Rees et al. Nat. Rev. Genet. (2018) 19 (12): 770-788; Anzalone et al. Nat. Biotechnol. (2020) 38:824-844.

Yet another exemplary suitable genome editing technology includes “prime editing,” which includes the introduction of new genetic information, e.g., an altered nucleotide sequence, into a specifically targeted genomic site using a catalytically impaired or partially catalytically impaired RNA-guided nuclease, e.g., a CRISPR/Cas nuclease, fused to an engineered reverse transcriptase (RT) domain. The Cas/RT fusion is targeted to a target site within the genome by a guide RNA that also comprises a nucleic acid sequence encoding the desired edit, and that can serve as a primer for the RT. See, e.g., Anzalone et al. Nature (2019) 576 (7785): 149-157.

The use of genome editing technology typically features the use of a suitable RNA-guided nuclease, which, in some embodiments, e.g., for base editing or prime editing, is catalytically impaired, or partially catalytically impaired. Examples of suitable RNA-guided nucleases include CRISPR/Cas nucleases. For example, in some embodiments, a suitable RNA-guided nuclease for use in the methods of genetically engineering cells provided herein is a Cas9 nuclease, e.g., an spCas9 or an saCas9 nuclease. For another example, in some embodiments, a suitable RNA-guided nuclease for use in the methods of genetically engineering cells provided herein is a Cas12 nuclease, e.g., a Cas12a nuclease. Exemplary suitable Cas12 nucleases include, without limitation, AsCas12a, FnCas12a, other Cas12a orthologs, and Cas12a derivatives, such as the MAD7 system (MAD7™, Inscripta, Inc.), or the Alt-R Cas12a (Cpf1) Ultra nuclease (Alt-R® Cas12a Ultra; Integrated DNA Technologies, Inc.). See, e.g., Gill et al. LIPSCOMB 2017. In United States: Inscripta Inc.; Price et al. Biotechnol. Bioeng. (2020) 117 (60): 1805-1816.

In some embodiments, a genetically engineered cell (e.g., a genetically engineered hematopoietic cell, such as, for example, a genetically engineered hematopoietic stem or progenitor cell or a genetically engineered immune effector cell) described herein is generated by targeting an RNA-guided nuclease, e.g., a CRISPR/Cas nuclease, such as, for example, a Cas9 nuclease or a Cas12a nuclease, to a suitable target site in the genome of the cell, under conditions suitable for the RNA-guided nuclease to bind the target site and cut the genomic DNA of the cell. In some embodiments, a genetically engineered cell (e.g., a genetically engineered hematopoietic cell, such as, for example, a genetically engineered hematopoietic stem or progenitor cell or a genetically engineered immune effector cell) described herein is generated by targeting a base editor, e.g., a CBE or ABE to a suitable target site in the genome of the cell, under conditions suitable for the base editor to bind the target site and cut the genomic DNA of the cell. A suitable RNA-guided nuclease can be targeted to a specific target site within the genome by a suitable guide RNA (gRNA). Suitable gRNAs for targeting CRISPR/Cas nucleases according to some aspects of this disclosure are provided herein and exemplary suitable gRNAs are described in more detail elsewhere herein.

In some embodiments, a gRNA that binds to a gene encoding a lineage-specific cell-surface antigen (e.g., a CD123 gRNA, CD38 gRNA, CD5 gRNA, CD47 gRNA, CD34 gRNA, EMR2 gRNA, or CD19 gRNA) described herein is complexed with a CRISPR/Cas nuclease, e.g., a Cas9 nuclease, a base editor. Various Cas9 nucleases and base editors are suitable for use with the gRNAs provided herein to effect genome editing according to some aspects of this disclosure, e.g., to create a genomic modification in the gene encoding a lineage-specific cell-surface antigen. Typically, the Cas nuclease or base editor and the gRNA are provided in a form and under conditions suitable for the formation of a Cas/gRNA complex, that targets a target site on the genome of the cell, e.g., a target site within the gene encoding a lineage-specific cell-surface antigen (e.g., a target site in a sequence that encodes an epitope bound by an agent that specifically binds the gene encoding a lineage-specific cell-surface antigen). In some embodiments, a Cas nuclease is used that exhibits a desired PAM specificity to target the Cas/gRNA complex to a desired target domain in the gene encoding a lineage-specific cell-surface antigen. Suitable target domains and corresponding gRNA targeting domain sequences are provided herein.

In some embodiments, a Cas/gRNA or base editor/gRNA complex is formed, e.g., in vitro, and a target cell is contacted with the Cas/gRNA or base editor/gRNA complex, e.g., via electroporation of the Cas/gRNA or base editor/gRNA complex into the cell. In some embodiments, the cell is contacted with Cas protein or base editor and gRNA separately, and the Cas/gRNA or base editor/gRNA complex is formed within the cell. In some embodiments, the cell is contacted with a nucleic acid, e.g., a DNA or RNA (such as an mRNA), encoding the Cas protein or base editor, and/or with a nucleic acid encoding the gRNA, or both.

In some embodiments, genetically engineered cells as provided herein are generated using a suitable genome editing technology, wherein the genome editing technology is characterized by the use of a Cas9 nuclease. In some embodiments, the Cas9 molecule is of, or derived from, Streptococcus pyogenes (SpCas9), Staphylococcus aureus (SaCas9), or Streptococcus thermophilus (stCas9). Additional suitable Cas9 molecules include those of, or derived from, Neisseria meningitidis (NmCas9), Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In some embodiments, catalytically impaired, or partially impaired, variants of such Cas9 nucleases can be used. Additional suitable Cas9 nucleases, and nuclease variants, will be apparent to those of skill in the art based on the present disclosure. The present disclosure is not limited in this respect.

In some embodiments, the Cas nuclease is a naturally occurring Cas molecule. In some embodiments, the Cas nuclease is an engineered, altered, or modified Cas molecule that differs, e.g., by at least one amino acid residue, from a reference sequence, e.g., the most similar naturally occurring Cas9 molecule or a sequence of Table 50 of International Publication No. WO 2015/157070, which is herein incorporated by reference in its entirety.

In some embodiments, a Cas nuclease is used that belongs to class 2 type V of Cas nucleases. Class 2 type V Cas nucleases can be further categorized as type V-A, type V-B, type V-C, and type V-U. See, e.g., Stella et al. Nature Structural & Molecular Biology (2017). In some embodiments, the Cas nuclease is a type V-B Cas endonuclease, such as a C2c1. See, e.g., Shmakov et al. Mol Cell (2015) 60:385-397. In some embodiments, the Cas nuclease used in the methods of genome editing provided herein is a type V-A Cas endonuclease, such as a Cpf1 (Cas12a) nuclease. See, e.g., Strohkendl et al. Mol. Cell (2018) 71:1-9. In some embodiments, a Cas nuclease used in the methods of genome editing provided herein is a Cpf1 nuclease derived from Provetella spp. or Francisella spp., Acidaminococcus sp. (AsCpf1), Lachnospiraceae bacterium (LpCpf1), or Eubacterium rectale. In some embodiments, the Cas nuclease is MAD7™.

Both naturally occurring and modified variants of CRISPR/Cas nucleases are suitable for use according to aspects of this disclosure. For example, dCas or nickase variants, Cas variants having altered PAM specificities, and Cas variants having improved nuclease activities are embraced by some embodiments of this disclosure. In some embodiments, the Cas nuclease is a variant having reduced PAM sequence specificity. In some embodiments, such a gRNA is referred to as “PAMless” or “near PAMless.” In some embodiments, the Cas nuclease is a SpRY nuclease. See, e.g., Walton et al., Science. 2020 Apr. 17; 368 (6488): 290-296, which is incorporated by reference herein.

Some features of some exemplary, non-limiting suitable Cas nucleases are described in more detail herein, without wishing to be bound to any particular theory.

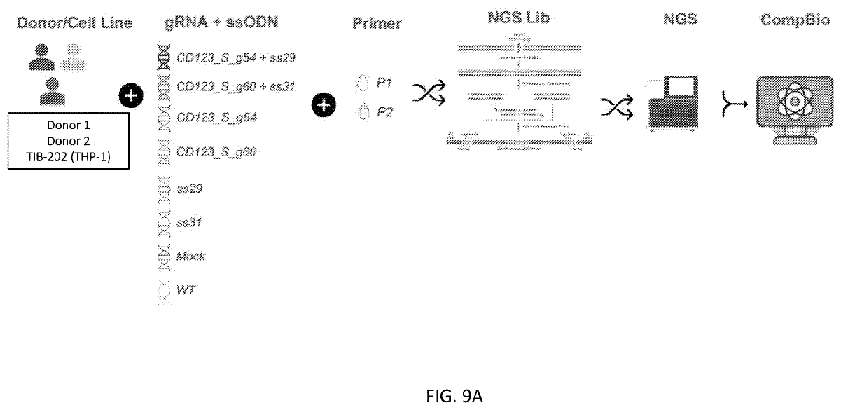

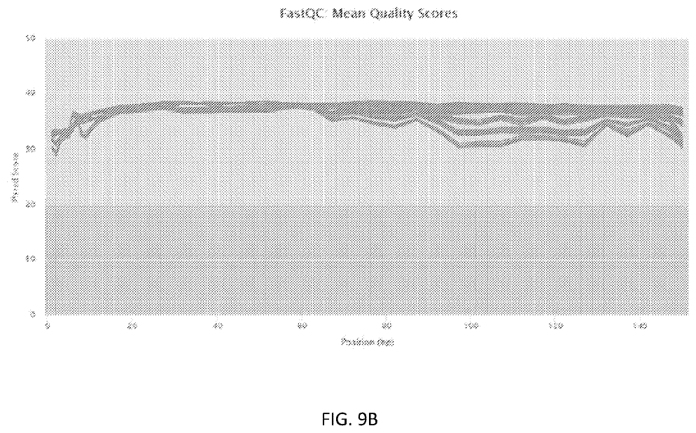

A naturally occurring Cas9 nuclease typically comprises two lobes: a recognition (REC) lobe and a nuclease (NUC) lobe; each of which further comprises domains described, e.g., in International Publication No. WO 2015/157070, e.g., in FIGS. 9A-9B therein (which application is incorporated herein by reference in its entirety).

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1 domain is involved in recognition of the repeat: anti-repeat duplex, e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat: anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule and comprises amino acids 1099-1368 of the sequence of S. pyogenes Cas9.

Crystal structures have been determined for naturally occurring bacterial Cas9 nucleases (see, e.g., Jinek et al., Science (2014) 343 (6176): 1247997) and for S. pyogenes Cas9 with a guide RNA (e.g., a synthetic fusion of crRNA and tracrRNA) (Nishimasu et al., Cell (2014) 156:935-949; and Anders et al., Nature (2014) doi: 10.1038/naturel3579).

In some embodiments, a Cas9 molecule described herein exhibits nuclease activity that results in the introduction of a double strand DNA break in or directly proximal to a target site. In some embodiments, the Cas9 molecule has been modified to inactivate one of the catalytic residues of the endonuclease. In some embodiments, the Cas9 molecule is a nickase and produces a single stranded break. See, e.g., Dabrowska et al. Frontiers in Neuroscience (2018) 12 (75). It has been shown that one or more mutations in the RuvC and HNH catalytic domains of the enzyme may improve Cas9 efficiency. See, e.g., Sarai et al. Currently Pharma. Biotechnol. (2017) 18 (13). In some embodiments, the Cas9 molecule is fused to a second domain, e.g., a domain that modifies DNA or chromatin, e.g., a deaminase or demethylase domain. In some such embodiments, the Cas9 molecule is modified to eliminate its endonuclease activity.

In some embodiments, a Cas nuclease or a Cas/gRNA complex described herein is administered together with a template for homology directed repair (HDR), e.g., as described herein. In some embodiments, a Cas nuclease or a Cas/gRNA complex described herein is administered without a HDR template.

In some embodiments, a Cas9 nuclease is used that is modified to enhance specificity of the enzyme (e.g., reduce off-target effects, maintain robust on-target cleavage). In some embodiments, the Cas9 molecule is an enhanced specificity Cas9 variant (e.g., eSPCas9). See, e.g., Slaymaker et al. Science (2016) 351 (6268): 84-88. In some embodiments, the Cas9 molecule is a high fidelity Cas9 variant (e.g., SpCas9-HF1). See, e.g., Kleinstiver et al. Nature (2016) 529:490-495.

Various Cas nucleases are known in the art and may be obtained from various sources and/or engineered/modified to modulate one or more activities or specificities of the enzymes. PAM sequence preferences and specificities of suitable Cas nucleases, e.g., suitable Cas9 nucleases, such as, for example, spCas9 and saCas9 are known in the art. In some embodiments, the Cas nuclease has been engineered/modified to recognize one or more PAM sequence. In some embodiments, the Cas nuclease has been engineered/modified to recognize one or more PAM sequence that is different than the PAM sequence the Cas nuclease recognizes without engineering/modification. In some embodiments, the Cas nuclease has been engineered/modified to reduce off-target activity of the enzyme.

In some embodiments, a Cas nuclease is used that is modified further to alter the specificity of the endonuclease activity (e.g., reduce off-target cleavage, decrease the endonuclease activity or lifetime in cells, increase homology-directed recombination and reduce non-homologous end joining). See, e.g., Komor et al. Cell (2017) 168:20-36. In some embodiments, a Cas nuclease is used that is modified to alter the PAM recognition or preference of the endonuclease. For example, SpCas9 recognizes the PAM sequence NGG, whereas some variants of SpCas9 comprising one or more modifications (e.g., VQR SpCas9, EQR SpCas9, VRER SpCas9) may recognize variant PAM sequences, e.g., NGA, NGAG, and/or NGCG. For another example, SaCas9 recognizes the PAM sequence NNGRRT, whereas some variants of SaCas9 comprising one or more modifications (e.g., KKH SaCas9) may recognize the PAM sequence NNNRRT. In another example, FnCas9 recognizes the PAM sequence NNG, whereas a variant of the FnCas9 comprises one or more modifications (e.g., RHA FnCas9) may recognize the PAM sequence YG. In another example, the Cas12a nuclease comprising substitution mutations S542R and K607R recognizes the PAM sequence TYCV. In another example, a Cpf1 endonuclease comprising substitution mutations S542R, K607R, and N552R recognizes the PAM sequence TATV. See, e.g., Gao et al. Nat. Biotechnol. (2017) 35 (8): 789-792. In another example, a SpG Cas9 endonuclease recognizes the PAM sequence NG (also referred to as a “PAM-flexible” PAM). In another example, a SpRY Cas9 endonuclease recognizes the PAM sequence NRN or NYN (also referred to as a “PAM-less” PAM) with higher efficiency where R is A or G and Y is a T or C. See, e.g., Liang et al. Nat. Comm. (2022) 13:3421; Walton et al. Science (2020) 368 (6488): 290-296. In some embodiments, a base editor (e.g., ABE or CBE) comprises an SpG Cas9 endonuclease. In some embodiments, a base editor (e.g., ABE or CBE) comprises an SpRY Cas9 endonuclease.

In some embodiments, a base editor is used to create a genomic modification resulting in expression of a variant of a gene encoding a lineage-specific cell-surface antigen not targeted by an immunotherapy. Base editors typically comprise a catalytically inactive or partially inactive Cas nuclease fused to a functional domain, e.g., a deaminase domain. See, e.g., Eid et al. Biochem. J. (2018) 475 (11): 1955-1964; Rees et al. Nature Reviews Genetics (2018) 19:770-788. In some embodiments, a catalytically inactive Cas nuclease is referred to as “dead Cas” or “dCas.”

In some embodiments, the catalytically inactive Cas molecule has reduced activity and is, e.g., a nickase (nCas). In some embodiments, the endonuclease comprises a dCas or nCas fused to an adenine base editor (ABE), for example an ABE evolved from the RNA adenine deaminase TadA. In some embodiments, the endonuclease comprises a dCas or nCas fused to a cytosine base editor (CBE), for example a CBE evolved from the cytidine deaminase enzyme (e.g., APOBEC deaminase, pmCDA1, activation-induced cytidine deaminase (AID)).

Examples of suitable base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa (KKH)-BE3, Target-AID, Target-AID-NG, xBE3, eA3A-BE3, BE-PLUS, TAM, CRISPR-X, ABE7.9, ABE7.10, ABE7.10*, ABE8, ABE8e, xABE, ABESa, VQR-ABE, VRER-ABE, Sa (KKH)-ABE, CBE, CBE1, CBE2, CBE3, CBE4, and CRISPR-SKIP. Additional examples of base editors can be found, for example, in US Publication No. 2018/0312825A1, US Publication No. 2018/0312828A1, and International Publication No. WO 2018/165629A1, which are incorporated by reference herein in their entireties.

Some aspects of this disclosure provide guide RNAs that are suitable to target an RNA-guided nuclease, e.g. as provided herein, to a suitable target site in the genome of a cell in order to effect a modification in the genome of the cell that results in expression of a variant form of a gene encoding a lineage-specific cell-surface antigen that is not recognized by an immunotherapeutic agent targeting a lineage-specific cell-surface antigen.

The terms “guide RNA” and “gRNA” are used interchangeably herein and refer to a nucleic acid, typically an RNA, that is bound by an RNA-guided nuclease and promotes the specific targeting or homing of the RNA-guided nuclease to a target nucleic acid, e.g., a target site within the genome of a cell. A gRNA typically comprises at least two domains: a “binding domain,” also sometimes referred to as “gRNA scaffold” or “gRNA backbone” that mediates binding to an RNA-guided nuclease (also referred to as the “binding domain”), and a “targeting domain” that mediates the targeting of the gRNA-bound RNA-guided nuclease to a target site. Some gRNAs comprise additional domains, e.g., complementarity domains, or stem-loop domains. The structures and sequences of naturally occurring gRNA binding domains and engineered variants thereof are well known to those of skill in the art. Some suitable gRNAs are unimolecular, comprising a single nucleic acid sequence, while other suitable gRNAs comprise two sequences (e.g., a crRNA and tracrRNA sequence).

Some exemplary suitable Cas9 gRNA scaffold sequences are provided herein, and additional suitable gRNA scaffold sequences will be apparent to the skilled artisan based on the present disclosure. Such additional suitable scaffold sequences include, without limitation, those recited in Jinek, et al. Science (2012) 337 (6096): 816-821, Ran, et al. Nature Protocols (2013) 8:2281-2308, International Publication No. WO 2014/093694, and International Publication No. WO 2013/176772.

For example, the binding domains of naturally occurring spCas9 gRNA typically comprise two RNA molecules, the crRNA (partially) and the tracrRNA. Variants of spCas9 gRNAs that comprise only a single RNA molecule including both crRNA and tracrRNA sequences, covalently bound to each other, e.g., via a tetraloop or via click-chemistry type covalent linkage, have been engineered and are commonly referred to as “single guide RNA” or “sgRNA.” Suitable gRNAs for use with other Cas nucleases, for example, with Cas12a nucleases, typically comprise only a single RNA molecule, as the naturally occurring Cas12a guide RNA comprises a single RNA molecule. In some embodiments, a suitable gRNA is unimolecular (having a single RNA molecule), sometimes referred to herein as sgRNAs, or modular (comprising more than one, and typically two, separate RNA molecules).

A gRNA suitable for targeting a target site in the gene encoding a lineage-specific cell-surface antigen can comprise a number of domains. For example, in some embodiments where a Cas9 nuclease is used, a unimolecular sgRNA, comprises, from 5′ to 3′:
a targeting domain corresponding to a target site sequence in the CD123 gene (e.g., a target site in or proximal to exon 3 and/or exon 4);
a first complementarity domain;
a linking domain;
a second complementarity domain (which is complementary to the first complementarity domain);
a proximal domain; and
optionally, a tail domain.

Each of these domains is now described in more detail.

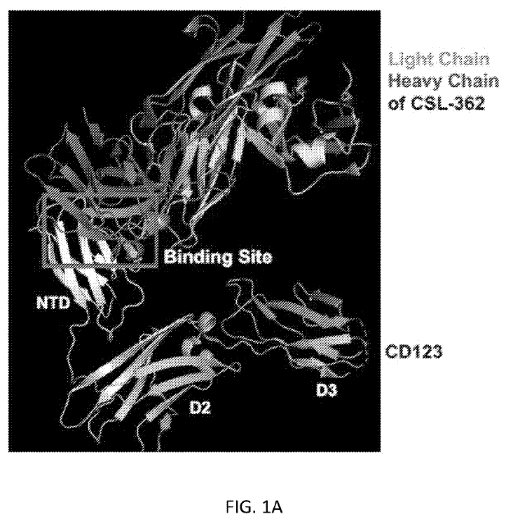

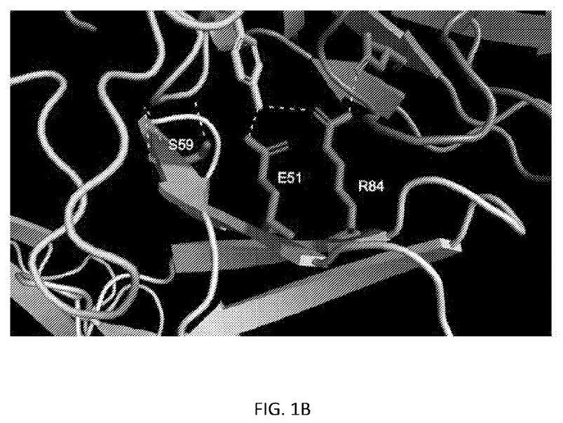

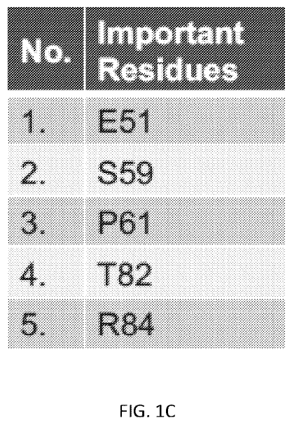

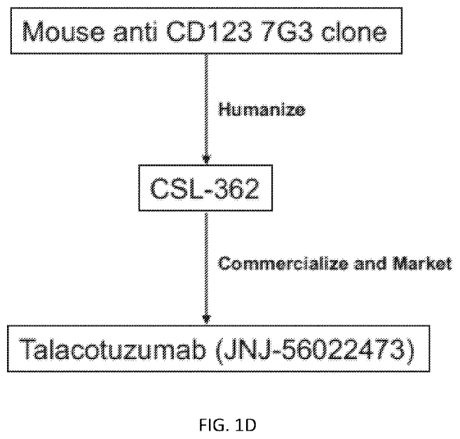

A gRNA as provided herein typically comprises a targeting domain that binds to a target site in the genome of a cell. The target site is typically a double-stranded DNA sequence comprising the PAM sequence and, on the same strand as, and directly adjacent to, the PAM sequence, the target domain. The targeting domain of the gRNA typically comprises an RNA sequence that corresponds to the target domain sequence in that it resembles the sequence of the target domain, sometimes with one or more mismatches, but typically comprises an RNA instead of a DNA sequence. The targeting domain of the gRNA thus base-pairs (in full or partial complementarity) with the sequence of the double-stranded target site that is complementary to the sequence of the target domain, and thus with the strand complementary to the strand that comprises the PAM sequence. It will be understood that the targeting domain of the gRNA typically does not include the PAM sequence. It will further be understood that the location of the PAM may be 5′ or 3′ of the target domain sequence, depending on the nuclease employed. For example, the PAM is typically 3′ of the target domain sequences for Cas9 nucleases, and 5′ of the target domain sequence for Cas12a nucleases. For an illustration of the location of the PAM and the mechanism of gRNA binding a target site, see, e.g., FIG. 1 of Vanegas et al., Fungal Biol Biotechnol. 2019; 6:6, which is incorporated by reference herein. For additional illustration and description of the mechanism of gRNA targeting an RNA-guided nuclease to a target site, see Fu Y et al, Nat Biotechnol 2014 (doi: 10.1038/nbt.2808) and Sternberg S H et al., Nature 2014 (doi: 10.1038/naturel3011), both incorporated herein by reference.

The targeting domain may comprise a nucleotide sequence that corresponds to the sequence of the target domain, i.e., the DNA sequence directly adjacent to the PAM sequence (e.g., 5′ of the PAM sequence for Cas9 nucleases, or 3′ of the PAM sequence for Cas12a nucleases). The targeting domain sequence typically comprises between 17 and 30 nucleotides and corresponds fully with the target domain sequence (i.e., without any mismatch nucleotides), or may comprise one or more, but typically not more than 4, mismatches. As the targeting domain is part of an RNA molecule, the gRNA, it will typically comprise ribonucleotides, while the DNA targeting domain will comprise deoxyribonucleotides.

An exemplary illustration of a Cas9 target site, comprising a 22 nucleotide target domain, and an NGG PAM sequence, as well as of a gRNA comprising a targeting domain that fully corresponds to the target domain (and thus base-pairs with full complementarity with the DNA strand complementary to the strand comprising the target domain and PAM) is provided below:

[target domain (DNA)] [PAM]
5′-N-N-N-N-N-N-N-N-N-N-N-N-N-N-N-N-N-N-N-N-N-N-N-G-G-3′ (DNA)
3′-N-N-N-N-N-N-N-N-N-N-N-N-N-N-N-N-N-N-N-N-N-N-N-C-C-5′ (DNA)
| | | | | | | | | | | | | | | | | | | | | |
5′-N-N-N-N-N-N-N-N-N-N-N-N-N-N-N-N-N-N-N-N-N-N-[gRNA scaffold]-3′ (RNA)
[targeting domain (RNA)] [binding domain]

An exemplary illustration of a Cas12a target site, comprising a 22 nucleotide target domain, and a TTN PAM sequence, as well as of a gRNA comprising a targeting domain that fully corresponds to the target domain (and thus base-pairs with full complementarity with the DNA strand complementary to the strand comprising the target domain and PAM) is provided below:

```
[PAM] [target domain (DNA)]
                    5′-T-T-N-N-N-N-N-N-N-N-N-N-N-N-N-N-N-N-N-N-N-N-N-N-N-3′ (DNA)
                    3′-A-A-N-N-N-N-N-N-N-N-N-N-N-N-N-N-N-N-N-N-N-N-N-N-N-5′ (DNA)
                                      | | | | | | | | | | | | | | | | | | | | | |
5′-[gRNA scaffold]-N-N-N-N-N-N-N-N-N-N-N-N-N-N-N-N-N-N-N-N-N-N-3′ (RNA)
[binding domain] [targeting domain (RNA)]
```

In some embodiments, the Cas12a PAM sequence is 5′-T-T-T-V-3′.

While not wishing to be bound by theory, at least in some embodiments, it is believed that the length and complementarity of the targeting domain with the target sequence contributes to specificity of the interaction of the gRNA/Cas9 molecule complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In some embodiments, the targeting domain fully corresponds, without mismatch, to a target domain sequence provided herein, or a part thereof. In some embodiments, the targeting domain of a gRNA provided herein comprises 1 mismatch relative to a target domain sequence provided herein. In some embodiments, the targeting domain comprises 2 mismatches relative to the target domain sequence. In some embodiments, the target domain comprises 3 mismatches relative to the target domain sequence.

In some embodiments, a targeting domain comprises a core domain and a secondary targeting domain, e.g., as described in International Publication No. WO 2015/157070, which is incorporated by reference in its entirety. In some embodiments, the core domain comprises about 8 to about 13 nucleotides from the 3′ end of the targeting domain (e.g., the most 3′ 8 to 13 nucleotides of the targeting domain). In some embodiments, the secondary domain is positioned 5′ to the core domain. In some embodiments, the core domain corresponds fully with the target domain sequence, or a part thereof. In other embodiments, the core domain may comprise one or more nucleotides that are mismatched with the corresponding nucleotide of the target domain sequence.

In some embodiments, e.g., in some embodiments where a Cas9 gRNA is provided, the gRNA comprises a first complementarity domain and a second complementarity domain, wherein the first complementarity domain is complementary with the second complementarity domain, and, at least in some embodiments, has sufficient complementarity to the second complementarity domain to form a duplexed region under at least some physiological conditions. In some embodiments, the first complementarity domain is 5 to 30 nucleotides in length. In some embodiments, the first complementarity domain comprises 3 subdomains, which, in the 5′ to 3′ direction are: a 5′ subdomain, a central subdomain, and a 3′ subdomain. In some embodiments, the 5′ subdomain is 4 to 9, e.g., 4, 5, 6, 7, 8 or 9 nucleotides in length. In some embodiments, the central subdomain is 1, 2, or 3, e.g., 1, nucleotide in length. In some embodiments, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus or S. thermophilus, first complementarity domain.

The sequence and placement of the above-mentioned domains are described in more detail in International Publication No. WO 2015/157070, which is herein incorporated by reference in its entirety, including p. 88-112 therein.

A linking domain may serve to link the first complementarity domain with the second complementarity domain of a unimolecular gRNA. The linking domain can link the first and second complementarity domains covalently or non-covalently. In some embodiments, the linkage is covalent. In some embodiments, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in International Publication No. WO 2018/126176, the entire contents of which are incorporated herein by reference.

In some embodiments, the second complementarity domain is complementary, at least in part, with the first complementarity domain, and in an embodiment, has sufficient complementarity to the second complementarity domain to form a duplexed region under at least some physiological conditions. In some embodiments, the second complementarity domain can include a sequence that lacks complementarity with the first complementarity domain, e.g., a sequence that loops out from the duplexed region. In some embodiments, the second complementarity domain is 5 to 27 nucleotides in length. In some embodiments, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24 or 25 nucleotides in length. In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In some embodiments, the central subdomain is 1, 2, 3, 4 or 5, e.g., 3 nucleotides in length. In some embodiments, the 3′ subdomain is 4 to 9, e.g., 4, 5, 6, 7, 8 or 9 nucleotides in length. In some embodiments, the 5′ subdomain and the 3′ subdomain of the first complementarity domain, are respectively, complementary, e.g., fully complementary, with the 3′ subdomain and the 5′ subdomain of the second complementarity domain.

In some embodiments, the proximal domain is 5 to 20 nucleotides in length. In some embodiments, the proximal domain can share homology with or be derived from a naturally occurring proximal domain. In an embodiment, it has at least 50% homology with a proximal domain from S. pyogenes, S. aureus, or S. thermophilus.

A broad spectrum of tail domains are suitable for use in gRNAs. In some embodiments, the tail domain is 0 (absent), 1, 2, 3, 4, 5, 6, 7, 8, 9, or 10 nucleotides in length. In some embodiments, the tail domain nucleotides are from or share homology with a sequence from the 5′ end of a naturally occurring tail domain. In some embodiments, the tail domain includes sequences that are complementary to each other and which, under at least some physiological conditions, form a duplexed region. In some embodiments, the tail domain is absent or is 1 to 50 nucleotides in length. In some embodiments, the tail domain can share homology with or be derived from a naturally occurring proximal tail domain. In some embodiments, the tail domain has at least 50% homology/identity with a tail domain from S. pyogenes, S. aureus or S. thermophilus. In some embodiments, the tail domain includes nucleotides at the 3′ end that are related to the method of in vitro or in vivo transcription.

In some embodiments, a gRNA provided herein comprises:
a first strand comprising, e.g., from 5′ to 3′:
a targeting domain (which corresponds to a target domain in a gene encoding a lineage-specific cell-surface antigen, e.g., a sequence encoding an epitope, e.g., described herein); and
a first complementarity domain; and
a second strand, comprising, e.g., from 5′ to 3′:
optionally, a 5′ extension domain;
a second complementarity domain;
a proximal domain; and
optionally, a tail domain.

In some embodiments, any of the gRNAs provided herein comprise one or more nucleotides that are chemically modified. Chemical modifications of gRNAs have previously been described, and suitable chemical modifications include any modifications that are beneficial for gRNA function and do not measurably increase any undesired characteristics, e.g., off-target effects, of a given gRNA. Suitable chemical modifications include, for example, those that make a gRNA less susceptible to endo-or exonuclease catalytic activity, and include, without limitation, phosphorothioate backbone modifications, 2′-O-Me-modifications (e.g., at one or both of the 3′ and 5′ termini), 2′F-modifications, replacement of the ribose sugar with the bicyclic nucleotide-cEt, 3′thioPACE (MSP) modifications, or any combination thereof. Additional suitable gRNA modifications will be apparent to the skilled artisan based on this disclosure, and such suitable gRNA modifications include, without limitation, those described, e.g., in Rahdar et al. PNAS (2015) 112 (51) E7110-E7117 and Hendel et al., Nat Biotechnol. (2015); 33 (9): 985-989, each of which is incorporated herein by reference in its entirety.

For example, a gRNA provided herein may comprise one or more 2′-O modified nucleotide, e.g., a 2′-O-methyl nucleotide. In some embodiments, the gRNA comprises a 2′-O modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., a 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g. 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g. 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, a gRNA provided herein may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, a gRNA provided herein comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

In some embodiments, a gRNA described herein comprises one or more 2′-O-methyl-3′-phosphorothioate nucleotides, e.g., at least 1, 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at one or more of the three terminal positions and the 5′ end and/or at one or more of the three terminal positions and the 3′ end. In some embodiments, the gRNA comprises one or more modified nucleotides, e.g., as described in International Publication Nos. WO 2017/214460, WO 2016/089433, and WO 2016/164356, which are incorporated by reference their entirety.

The gRNAs targeting a gene encoding a lineage-specific cell-surface antigen provided herein can be delivered to a cell in any manner suitable. Various suitable methods for the delivery of CRISPR/Cas systems, e.g., comprising an RNP including a gRNA bound to an RNA-guided nuclease, have been described, and exemplary suitable methods include, without limitation, electroporation of RNP into a cell, electroporation of mRNA encoding a Cas nuclease and a gRNA into a cell, various protein or nucleic acid transfection methods, and delivery of encoding RNA or DNA via viral vectors, such as, for example, retroviral (e.g., lentiviral) vectors. Any suitable delivery method is embraced by this disclosure, and the present disclosure is not limited in this respect.

The present disclosure provides a number of CD123 target sites and corresponding gRNAs that are useful for targeting an RNA-guided nuclease to human CD123. Table 1 below illustrates preferred target domains in the human endogenous CD123 gene that can be bound by gRNAs described herein. The exemplary target sequences of human CD123 shown in Table 1, in some embodiments, are for use with a Cas9 nuclease, e.g., SpCas9.

TABLE 1
Exemplary Cas9 target site sequences of human CD123 are provided, as are
exemplary gRNA targeting domain sequences useful for targeting such sites.

| gRNA Name | gRNA Alternate Name | Target domain sequence |
|---|---|---|
| guide-29 | guide-54 | CACATTTCTGTTAAGGTCCC (SEQ ID NO: 1) |
| | | GGGACCTTAACAGAAATGTG (SEQ ID NO: 2) |
| | | CACAUUUCUGUUAAGGUCCC (SEQ ID NO: 3) |
| guide-30 | guide-56 | TATCGGTCACATTTCTGTTA (SEQ ID NO: 4) |
| | | TAACAGAAATGTGACCGATA (SEQ ID NO: 5) |
| | | UAUCGGUCACAUUUCUGUUA (SEQ ID NO: 6) |
| guide-31 | guide-60 | GTCTTTAACACACTCGATAT (SEQ ID NO: 7) |
| | | ATATCGAGTGTGTTAAAGAC (SEQ ID NO: 8) |
| | | GUCUUUAACACACUCGAUAU (SEQ ID NO: 9) |
| guide-32 | guide-61 | AGACGCCGACTATTCTATGC (SEQ ID NO: 10) |
| | | GCATAGAATAGTCGGCGTCT (SEQ ID NO: 11) |
| | | AGACGCCGACUAUUCUAUGC (SEQ ID NO: 12) |

For each target site, the first sequence represents the DNA target domain sequence, the second sequence represents the reverse complement thereof, and the third sequence represents an exemplary targeting domain sequence of a gRNA that can be used to target the respective target site.

The present disclosure provides exemplary CD123 targeting gRNAs that are useful for targeting an RNA-guided nuclease to human CD123. Table 2 below illustrates preferred targeting domains for use in gRNAs targeting Cas9 nucleases to human endogenous CD123 gene. The exemplary target sequences of human CD123 shown in Table 2, in some embodiments, are for use with a Cas9 nuclease, e.g., SpCas9.

TABLE 2
Exemplary Cas9 targeting domain sequences of
gRNAs targeted to human CD123 are provided.

| | gRNA | |
|---|---|---|
| | Alternate | |
| gRNA Name | Name | Targeting domain sequence |
| guide-29 | guide-54 | CACAUUUCUGUUAAGGUCCC |
| | | (SEQ ID NO: 3) |
| guide-30 | guide-56 | UAUCGGUCACAUUUCUGUUA |
| | | (SEQ ID NO: 6) |
| guide-31 | guide-60 | GUCUUUAACACACUCGAUAU |
| | | (SEQ ID NO: 9) |
| guide-32 | guide-61 | AGACGCCGACUAUUCUAUGC |
| | | (SEQ ID NO: 12) |

TABLE 3
Exemplary targeting domain sequences of gRNAs targeted to human CD123 using
base editors (e.g., ABE or CBE) comprising SpRY Cas9 or SpG Cas9 are provided.

| | Targeting | PAM | BE | | Amino | |
|---|---|---|---|---|---|---|
| gRNA Name | domain sequence | Sequence | Sequence | Codon | Acids | Consequence |
| CD123_g101 | ACCGATATCGAGTGTGTTAA | AG | CGgTaT | GAT/GGT | D->G | Missense |
| | (SEQ ID NO: 100) | | CGgTgT | GATATC/ | DI->GV | variant |
| | | | | GGTGTC | | |
| CD123_g104 | GATATCGAGTGTGTTAAAGA | CG | TgTCGa | ATC/GTC | I->V | Missense |
| | (SEQ ID NO: 101) | | TgTCGg | ATCGAG/ | IE->VG | variant |
| | | | | GTCGGG | | |
| CD123_g105 | ATCGAGTGTGTTAAAGACGC | CG | CGgGTG | GAG/GGG | E->G | Missense |
| | (SEQ ID NO: 102) | | | | | variant |
| CD123_g100 | AATGTGACCGATATCGAGTG | TG | TGTGAt | ACC/ATC | T->I | Missense |
| | (SEQ ID NO: 103) | | | | | variant |
| CD123_g103 | GTCTTTAACACACTCGATAT | CG | tTTTAA | GAC/AAC | D->N | Missense |
| | (SEQ ID NO: 7) | | | | | variant |
| CD123_g98 | CTCGATATCGGTCACATTTC | TG | tGATAT | GAG/ AAG | E->K | Missense |
| | (SEQ ID NO: 104) | | | | | variant |
| CD123_g | TCGAGTGTGTTAAAGACGCC | GAC | GgGTGT | GAG/GGG | E->G | Missense |
| | (SEQ ID NO: 195) | | | | | variant |
| CD123_g | ATAGAATAGTCGGCGTCTTT | AAC | gGaaTa | TATTCT/ | SS->P | Missense |
| | (SEQ ID NO: 196) | | gGgaTa | CACCCC | YS->YP | variant |
| | | | gGggTa | | YS->HP | |
| | | | gGggTg | | | |

A representative DNA sequence of the CD123 gene is provided by NCBI Gene ID: 3563, shown below.

(SEQ ID NO: 13)
AACATGATAATTTTCAAAGAAAGAGTCTTTCTTTCAAGGAAAGTCAGGTTCATGGTTACGAAGCTGCTGACCCCA
GGATCCCAGCCCGTGGGAGAGAAGGGGGTCTCTGACAGCCCCCACCCCTCCCCACTGCCAGATCCTTATTGGGTC
TGAGTTTCAGGGGTGGGGCCCCAGCTGGAGGTTATAAAACAGCTCAATCGGGGAGTACAACCTTCGGTTTCTCTT
CGGGGAAAGCTGCTTTCAGCGCACACGGGAAGATATCAGAAACATCCTAGGATCAGGACACCCCAGATCTTCTCA
ACTGGAACCACGAAGGCTGTTTCTTCCACACAGTACTTTGATCTCCATTTAAGGTAAGGTCCCCCCTCCAGGGTG
GGATGAGGGAAAAAGAGGGCAGGCAGGGGAGGCGGTGGACTGTGGGTTCCCAAATCCAAGCTGGCAGACACGGGG
CATTGGCATGTAACAGGTTTCTGCTGAGACCTGCTGTCCGTGTGGGCACCACACCACACATAGAATGACCCGGTA
CCAGAGAAGTATGTTTGCGATCTCATTCACCCTTTTGTAAGTGGATCGGACGCTGAAGCCATCATAGCATGTTAG
ATGCCTTGGGTTTATAAAGGTGATAGCATTAGGAGTAATTTGACAGTGCTTTCTGCTTTTTCTGTGTGCTTGCTA
GAAAATGTACACACCTACACATGGCTAGAGTTCTCTGTTTATTAGAAAGCGCTGGTTCAGTTAGTCAATGAGCTC
ACGCCCAGGGCGAGCTGACTGCCAGCCGGGGAGCCGCTTTTGGGGCTGGGACCCCAGCCTGCTTGTAGTGTTTCT
CAGTGGTTTTCAAAGGCTGGCGGGACTCAGAATGTTCCAGATGGCTGGTGAAACCTCAGGTCCCTCCTAGCAGCT
TTATTCACAGTAGCCAAGAGGTGGAGACAGCCCTCGTACCCATCTATAGATGAATGGAAAAATATAATGTCCATC
CACACAGTGGAATATTACGCAGCCACGTTTCTCCTGCAGCAGGCACCTCTGTCCTGCGTTCCGGAGCTGCGTTCC
CGATGGTCCTCCTTTGGCTCACGCTGCTCCTGATCGCCCTGCCCTGTCTCCTGCAAACGAAGGAAGGTAAGAACT
GGAGAAAAAATGCACGTGCCACCTGGGGAGCGGTGGGGGTAGACAGACACACAATGTCAGCGTGCCGTCCTTCAG
GGAAACTTTTCATGCTGAGCTCATGGCAGAGTCTCATGCAGTGGTCGGGAATGACTCAGACACTTCCCTGTACCC
GTCACCAAGTTCCCTGTAATGCAGCATCTTGCAAAACGGTAATACGACCTCACAGGCAGGGACCTGATCGTGACA
CAGATGCCATGTGAGGTGTTTTGATGAAACTCACACGCTGGGATCAACAGCACCAATAACAATTTCCAGTTTCCT
TCATTGTTTATCTTACTTTTTCATTTTCTTATTATTTTTATTTTGGAGACAGGGTCTTACTGTCTTGCCCAGGCT
GGAGTGCAATAGTGTGATCTCGGCTCACTGCAACCTCTGCCTCCCGGATTCAAGCGATTCTTCTGCCTCAGCCTC
CCGAGGAGCTGAGATTACAGTCGCGCACCACCATACCCGGCTAATTTCTGTATTTTTGATAGAGACGGGATTTCA
CTATGTCGGTCAGGCTGGTCTAGAACTGCTGACCACAAGTGATCCGTCTGCCTTGGCCTCCCAAAGTGCTGGGAT
TACAGGTGTGAGCCACAGCGCCCGGCCTTTTTTTTCATTGGTTTTTACAGTCTATTACTGCAGGGTTTCACTTTA
CCTTGAATTTCTTTTAACTTTAATTTGCTTTTCATTCTTTAACTTCTTTTTTTTTTTTTTTTTTGACACAGAGTT
TCATTCTGGTCGCCCAGGCTGGAGTGTAATAGCGTGACCATGGCTCACTGCAACCTCTACCTGCTGGGTTCAATT
GATTCTCCTGTCTCAGCCTCCCAAGCACCTGGGATTACAGGTGTCCGCCACCACGCCCAGCTAATTTTTCTGTTT
TTACTAGAGACGGGGTTTCACCGTGTTAGACAGGATGGTCTCGATCTCCTGACCTCATGATCCGCCTGCCTCGGG
GTTGGGATTACACACTTTGGGAGGCCAAGGCAGGTGGACGATCACAAGGTCAGGAGTTCGTGACCAGCCTGACTA
ACACGGTGAAACCCCGTCTCTACTAAAAATACAAAAATCAGCTGGGCGTGGTGGCGGGCGCCTGTAATCCCAGTT
ACTCGGGAGGCTGAGGCAGGAGAGTCGCTTGAACCCGGGAGGAGGAGGTTGCAGTGAGCCTAGATCACGCCATTG
CACTCCAGCCTGGGCGACAGAGTGAGACTCCGTCTCAAAATGAATGAATGAATGAATGAATTTCTTATAAGAATT
TTTTTCCCCAGACAGTTTGTTTTAAGGGATAAATTATCCTTCTAAGTTAGAAGAAAATAATGCCAGAAGTCTAGA
CATTCTTATGCTTTGGTCCCGCTTATCAAACCAAGGTTGCTGACCTTGATAACCACTCAAGATCCTTACAGTTTA
TAAAGTCATTTCCTCAAGTTTTCTAAGTGGCCGATCAGAGATAAACCCTAGAGAAATAGTTGATGTATGTTTCTA
GCTTTGGGTGACCAGCAAAATGTGATAGAATATTGCCTTTTACTGGCCGGGTGCAACGGCTCACGTCTGTAATCC
CAGCACTTTGGGAGGCTGAGGCGGGTGGATCACTTGAGGTCAGGAGTTCAAAACCAGCTTGGCCATCATGGTGAA
ACCCCGTCTGTACTAAAAATACAAAAAAATTAGCTGGGCGCGGTGCTGTGCACCTGTAATCCCTGCTACTCAGGA
GGCTGAGGCAGGAGAATCACTTGAACCCGGGAGGCGGAGGTTGCCGTGAGCCAAGATCACGCCATTGCACTCCAA
CCTGGGTGACAGAGCGAGGCTCCATCTCAAAAAAAAAAAAAAAAAAAGAATATTGCCTTTAACATCTTTGTACAG
GTCATTTATGAAATATCTTGAGCTCTGTGATGGCTAAGAGAGACCTTCTTTTTCTTTCTTTCTTTTTTTTTTTTT
TGAGACGGAGTTTTTTTGTTTTTTTGAGATGGAGTCTCGCTCTGTTACCCAGGCTGGAGTGCAGTGGCACGATCT
CAGCTCACCGCAACCTCCGCCTCCCGAGTTCCAGTGATTCTCCTGCCTCAGCTTCCTGATTAGCTGGGATTACAG
GCGCCCGCCACCACGCCCAACTAATTTTTGTATTTTTAGTCGCGACGGGGTTTCACCGTGTTAGCCAGGATGGTC
TTGATCACTTGACCTGGTGATCCGCCCACCTTGGCCTCCCAAAGTGCTGGGATGACGGGCGTGAGCCACCACGCC
CGTTTGCTTTATGACTTCTACCAGCTCACAGAAGTCTCCTGTGTACATAGAACTCCACTTCCCAGCCAGGCTCAG
TAACTCACGTCTGTGATCCCAGCACTTTGGGAGGCTGAGGCAGGCAGATCATGTGAGGTTGGGAGTTCGAGACCA
GCCTGGCCAACATGGTGAAACCCCATCTCTAGTAAAAATACAAAAATTAGCCGAGTGTGGTGGCAGGCACCTGTT
ATCCCAGCTGCACAGCAGGCTGACACAGGATAATCGCTTGAACCCGGGAGGCGGAGGTTGTAGTGAGCCGAGATC
GCGCCACTGAATTCCAGCCTGGGCGACAGAGTGAGACTCCGTCTCAAAACAAACAAGCAAACAAAAATACCCATT
ACAATGTTGTTTTAAGATTGTTGTATATCAACTGGGCATGGTGGCTCATGCTTGTAATCCCAGCACTTTGAGAGG
CCGAGGCGGACAGATCACGAGGTCAGGAGATCGAGACCATCCTGGCCAACATGGTGAAACCCCATCTCTAGTAAA
AATACAAAAATTAGCCGGGCGTGGTGGTGGGCTCCTGTAATCCTAGCTACTCGGGAGGCTGAGGCAGGAGAATCG
CTTGAACCCGGGAGGTAGAGGTTGCGGTGAGCTGAGATCGTGCCACTGCACTCCAGCCTGGGTGACAAGAGCAAA
ACTCCGTCTCAAAAAAATAAAAAATTTAAAAAAATAAAGAACTCGACCTCCCAAAGGTATTGGCTAACTCCACGG
GCAAAAAAACCATACCCATTACAATGCTGTTTTAAGATTGTTGACCTGGTGCGGTGGCTCATGCCTGTAATCCCA
GCACTTTGGGAGGCTGAGGCGGACGAATCATGAGGTCAGGAGATCGAGACCATCCTGGCTAACACGGTGAAACGC
CATCTCTACTAAAAATACAAAAAAAAAAAAATTAGCCGGGCGTGGTGGCGGGCGCCTGTAGTCCCAGCTACTCGG
GAGGCTGAGGCAGGAGAGTTGCTTGAACCAGGAGGCGGAGCTTGCAGTGAGCTGAGATCGTGCCACTGTAGTCCA
GCCAGGGCGACAAGAGTGAAACTCCATGTCAAAAAATTTAAAAAAATTAAATAAAAGAACTCCACCTCCCAAAGG
TATTGGCTAACTCCACGGGCAAAAAAAAAAAACCATACCCCTTACAATGCCGTTTTAAGATTCTTACGTATCTCT
TCGAACTCCAACCTGTCACCGTTTTAGATCCAAACCCACCAATCACGAACCTAAGGATGAAAGCAAAGGCTCAGC
AGTTGACCTGGGACCTTAACAGAAATGTGACCGATATCGAGTGTGTTAAAGACGCCGACTATTCTATGCCGGTAA
ATCATACTCTCTATTGTTTTTTTATTTTTATTTTATTTATTTATGTATTTATGTATTTATTTATTTTTTGAGACG
GAGTCTTGCTCTGTCGCCCAGGCTGGACTGCGGTGACCCGATCTCCGCTCTCTGCAACCTCCACCTCCCAGGTCC
ATGCCATTCTCCTGCCTCAACCTCCCGAGTAGCTGGGACTACAGGCGCCCGCCACCATGCCCGGCTAATTTTTTT
GTATTTTTAGTAGAGATGGGGTTTCACTGTGTTAGCCAGGAGGGTCTCGATCTCCTGACCTCGTGATCTGCCCGC
CTCGGCCTCCCAAAGTGCTGGGATTACAGGCGCGAGTCACCGCGCATGGCCCAGACTCTCTAATGTTGACGAACA
AGACGTTTCCGTCTTCTGCAGGAATCTCAGAACCAATACTGCTCCCATCGCTGGTGTCATGACTACCTGGTTTCT
GCCCCGAAGTCAGGTGTGGGATTTGAAGTGACTTTGGAGGGTCTGGTTCCCCGTCCGCGGGACATCTAAGATGGC
ACACACTGGACAGGGTGGATGTGAAAGTGAAATGAGGCTAAGCTATGACTGGTGCGAAACCCAACCCCACGCTGG
GCGTGGTGGCTCACGTCTGTAATCCCAGCACTTTGGGAGGCTGAGGCGGGCGGATCATGAGGTCAGGAGTTCGAG
ACCAGTCTGGCCAACACGGTGAAACGCTGTCTCTGCTAAAAATACGAAAGTTAGCCGGGCACAGTGGCTCACACC
AGCACTTTGAGAGGCTGAGGCGGATGGATCACCTGGGGCCAGGAGTTCGAGACTAGTCTGGGCAACATGGTGAAA
CCCCGCCTCCACTAAAAATATAAAAATTAGCTGGGCATGGTGGTGGGCGCCTGTAATCCCAGTTACTTGGGAGGC
TGAGGCAGGAGAATCGCTTGAACCCGGGAGGCAGAGGTTGCATTGAGCCGAGACTGTGCCACTGCACTCCAGCCT
GGGGGACGAGAGCAAGACTTCATGTCAAAAAAGAAAAAGAAAAATTAGCCGGATGTGGTGGCACATGCCTGTAAT
ACCAGCTATTCAGGAGGCTGAGGCAGGAGAATTGCTTGAACCTGGGAGGCCGAGGTTGCACTGAGCCGAGATTGT
GCCACTGCACTCCAGCCTGGGTGACAGAGTGAGACTCCATCTCAAAACAAAAACAAAAACAAAAGCAAAAACAAA
ACAAAAGTGTGTGCTCAGGAAACAAGGTCCTCATCACGAAATCCTTCCAAATCCCCCATCTTGTCATCACCTGCG
TTCTCAGGGTTTGAGAACAGCGCCAGACCTCATGGGGTGGCCCAGGTGACACTGTGAGCTATTTACAAGTCAGTG
TCTTATGGGAAAGGAGCACGTTTCCCTGAGAACCTATTTGGTCCCCTCCAAGAGCTATGTTCGTTCAATACAATT
CAAATCACGGCCCTTCATGCGTCTGCTCGGGCCACCATTATAAAATCCTACCCCCAGCTCCAAATACAGTCCCAT
TGAACTTTGTGATTTTGGAGAGTAGAGATAAAACAGTCTAGAATCCCAGAGCGATTTTACCATACCATGGCAAAC
TGACTCTCAACTTTAGAAACACAAATGCTGAAAAAAAAACTAAGGAAATTTTGAAAAAGAAGGTGAATGAAGGAG
AACCTGCCTTACCTATATCAAAAGGCACTGAAAAGTTCATACCCAATGTGCGGATTGCTATAAGAATACACAAGT
AGGCCGGGCGTGGTGGCTCACGCCTGTCATCCCAGCACTTTGGAGGCCGAGGCGGGTGGATCACGAGGTCAGGAG
ATCGAGACCCTCCTGGCTAACACGGTGAAACCCCGTCTCTACTAAAAATATAAAAATTAGCCGGGCGTGGTGGCT
GGTGCCTGTAGTCCCAGCTACTCGGGAGGCTGAGGCAGGAGAATGGCTTGAACCTGGGAGGCAGAAGCTTGCAGT
GAGCCGAGATCGCGCCACTGCACTCCAGCCTGGGCGACAGAGCGAGACTCTGTCTCAAAAAAACAAAAACAAAAG
CAAACAAAACGAAGAATATACAAGTAGATTAATGAAATGTGGCCGGGTGCGGTGGTGAGGCAGGAGAATTGCTTG
AACCCGAGAGGTGGAGGTTGCAGTGAGCTGAGATCGCACCACTGAAGTCTAGCCTGGGCAAGCGGAGTGAGGCCC
TGTCTTAACAAAAAAACAAAGAAACAAAAAACAAACAAACAAGAAAAAAACAAAGCAAAACAAACAGAAAAGTAT
TTCACTAATATTTACTGCTAAGTGGGATTATTTTTATTCAAGCTTTTGTATCTTTAGAAAAAAATTGTGGCCGGG
CGCGGTGGCTCACGCCTGTCATCCCAGCACTTTGGGAGGCCGAGGAGGGTGGATCACGAGGTCAGGAGATCGAGA
CCCTCCTGGCTAACACGGTGAAACCCCGTCTCTACTAAAAATATAAAAATTAGCCGGGCGTGGTGGCGGGCGCCT
GTAGTCCCAGCTACTTGGGAGGATGAGGCAGGAGAATGGCATGAACCCGGGAGGGAGAGGCTACAGTGAGCCGAG
ATCGCGCCCCTGCACTCCAGCCTGGGCGACAGAGCGAGACTCCCTCTCAAAAAAAAAAAAAACAGAAAAAAGTCT
TGGCCGGGCACGGTGGCTCACGCCTGTAATCCCAGCACTTTGGGAGGCTGAGGCAGGCGGATCACATGAGGTCAG
GAGTTCGAGACCAGCCTGACCAACATGGTGAAACCCTGTCTCTACCCAGAAAAATACTTTAAAAATTAGCTGGGC
GTGGTGGCGGGCACCTGTAATCCCAGGTGCTCGGGAGGTTGAAGCAGGAGAATGGCTTGAACCCGGGAGGGAGAG
GCTGCAGTGAGCCGAGATCACGCCACTGCACTCCAGCGTGGGCGACGAGAGCGAAACTCTGCCTCAAAAAAAATC
TGAACATCATTAGCGTCAAATTAAGCATGGTCTGTCAGCAGCCATCATAGTCCTATGTCTCTCTTAGGCAGTGAA
CAATAGCTATTGCCAGTTTGGAGCAATTTCCTTATGTGAAGTGACCAACTACACCGTCCGAGTGGCCAACCCACC
ATTCTCCACGTGGATCCTCTTCCCTGAGAACAGTGAGAAAAATGTTCATTGTTTGTTTATTCTCTATTCCCTCCC
TCCTTCCCTCTCTCCCTCCCTCTCGCCTTCGCTGTGTCTTTTTTCTTTTCTTTTTCTCTTTCTTTCTTTCTTTCT
TTCTTTCTTTCTTTCTTTCTTTCTTTCTTTTTCTTTCTTTCTGTTTCTGTTTCTTTCTTCCTTTCTTTTTCTTTC
TTTCTTTCCTTCTTTCCTCTCTTTCTTTTCTTTCTCTTTCCCTCCCTCCCTTCTTTCTTTTCTTCACTTCCTTCC
CTCCCTCCTTCTCTCCTTTTCCTCCCTCCTCCTTCCCACCCTACTTCCTCTCTCTCCTTCCTTCCCTTTCGTTTT
CTTTTCCTCCCTGCCTCACTCCCTTCCTTCCTTCTCTCCCTCCTCTTTTCCTTCCTTCTTCCCTCCTTCCCTTTC
TCTCTCTCTCTCTTTCTCTCTTTCCCCCTCCCCTCCCTTCCCCTCCTCTCCCCTCTCCTTTCCTGACACGGTCTT
GCTCTGTTGCCCAGGCTGGAGCGCAGTGGTGCAATCACAGCTTACTGCAGGCTTCACCTCCTGAGCTCAAACAAT
CCTTCTGCCTCAGCCTCCCACGTAGGTGGAACTACACCCATGTACCACCATGCCCACCTAATTTTTTAAACACCT
TTTTTTTTTTTTAGACAGAGTCTCACTCTGTCCACCAGGCTGGAGTGCACTGGCGCGATCTCGGCTCACTGCAAC
CTCTGGCTCCCGGGTTCAAGCGATTCTCCTGCCTCAGCCTCCCGAGTAGCTGGGATTATAGGCGCCCACCGCCAC
GCCCAGCTAATTCTTGTATTTTTAGTAGAGATGGGGTTTCACCATGTTGGTCAGGCTGGTCTCAAACTCCTGACT
TCGTGATCTGCCCGCCTCGGCCTCCCAAAGTGCTGGGAGTACAGGGGTGAGCCACCGCGCCCAGCCTTAAATACT
TTTTGTAGAGATGGGGTGTCGCTATGTTACCTGAGCTGGTCTCAAACTCCTAGGCTCAAGTGATCCTCCCGCCTC
AGTCTCCCAAAGTGCAGGGATTGTAAGCATCAGCCACCACGCCTGGCCTCTTCTTTGTTTTTTAATTAATTAGAA
GTCCATGGTTTATTTTTTTGTTTTGTTTTGTTTGTTTTTCTGTGATGGAGTCTTGTTCTTGTCGCCCAGGCTGGA
GTGCAGTGGTGCGATTTCAGCTTACCGCAAACTCCGCCTCCCGGGTTCAAGCGATTCTCCTGCCTCAGCCTCACA
AGTAGCTGGGATTACAGGCACCCGCCACCAAGCCCGGCTAAATTTTGTATTTTAAGTAGAGACGGGGCTTCACCA
TGTTGGTCTCGAACTCCTGACCTTGTGATCCACCCGCCTCAGCCTCCCAAAGTGCTGGGATGACAGGCGGGAGCC
ACTGTGCCGGGCCTCAATTTAATTATTTTTAAGTTTAATGGATTTCCAGGGAGTTATGCCGAGTCGGAAAAAAAA
GACCACTCCAGAGCGTGACACACACAGTGATTTCATTAATACAACTGTCTTGAAATGGCAAAATTTTACAAATAG
AAAACAGTCTCCTGGGTTGCAGGGATATAAGCAGGAGTGAGAGCTGAGAGAGGTGGGTTTGGCTGTAAAAGTGCT
CCTTGGGGAGGCCGGGCGCAGTGGCTCACGCCTGTAATCCCAGCACTTTGGGAGGCCGAGGCGGGCGGATCACCT
GAGGTCAGGAGTTCGAGACCAGCCTGGCCAACATGGTGAAACCCCGTCTCTACTAAAAACACAAAAATTAGCCGG
GCATGGTGGCTCACGCCTGTAATCCCAGCACTTTGGGAGGCCGAGGCAGGCGGATCACCTGAGGTCAGGAATTCG
AGACCAGCCTGGCTAACATGGTGAAACCCCATCTCTACTAAAAATACAAAATTAGCCAGGGGTGGTGGCACATGC
CTGTAGTCCCAGCTACTCAGGAGGCTGAGGCAGGAGAATCACTTGAACCCGGGAGGCGGAGATTGCGGTGAGCTG
AGATCGTGCCATTGCTCTCCAGCCTGGGCAACAAGAGTGAAATTCCATCTAAAAAAAAAAAAAAATTAAAAAGAA
ATTAGCGGCCGGGCCCAGTGGCTCACACCTGTAATCCCAGCACTTTGGGAGGCCGAGGTGGGTGGATCGCGAGGT
CAGGAGATCGAGACCATCCCGGCTAACATGGTGAAACCCCATCTCTACTAAAAATACAAAATTAGCCAGGGGTGG
TGGCACATGCCTGCAGTCCCAGCTACTCAGGAGGCTGAGGCAGGAGAATCACTTGAACCTGGGAGGCGGAGGTTG
TGGTGAGCCATTGAGCTCCAGCCTGGGCAACAAGAGTGAAACTCTGTCTTAAACACACACACAGATGCACACACA
CGCACACACACACAAACAATGAAAGAAAAACATCTGTAGTCCCAGCAGTTTGGGAGGCTGAGGCGGGTGGATCAC
TTGAGGTCAGGAATTCGCGACCAGTCTGGGCAACATGGTGAAACCCCATCTCTACTAAAAATACAAAAATTAGCT
GGGTGTGGTAGTGGGTGCCTGTTGTCCCAGATACTTGGGAGGCTGAGGCAGGAGAATTGCTTGAACCCGGGAGGT
GGAGGTTGCAGTGAGCCGAGATGGCGCCATTGTACTCCAGCCTAGGCGACGGAGCGAGACTCTGTAAAAATGAAT
AAATAAATCATTAAAAGAATATGCAGGATCTCTCCATATTATTATTATTTTTTACAACGGCTTGTGTATCTACCA
TGGTCTCAAAATACAAAAGGCACTCTAAAAAGGAATTAGAGTTTTATTTTTTTTTTTGAGACGGAGTCTCGCTCT
TGTCACACAGGCTGGAGTGCAGTGACGTGGCCTGGGCTCACTGCAACCTCTGCCTCCTGGGTTCAAGAAATTCTC
CTGCCTCAGCCTCCCTAGTAGCTGGGATTACAGCGGCCCGACATCACGCCCGGCTAATTTTTGTATTTTTAGTAG
AGGCAGGGTTTCACCATGTTAGCCAGGCCGGTCTCGAACTCCGGACCTCAGGTGATCCTCCTGCCTCGGCCTCCC
AAAGTGCTGGGATTACAGGCGTGAGCCACCATGCCTGGGTTTGTTTTTTTATTTTTTTGAGACGAAGGCTCACTC
TGTCGCCCAGGCTGGAGTGCAGTGGCGTGATCTCAGCTCCATGCAACCTCTGCCTCCTGGGTTCAAGAAATTCTC
CTGCCTCAGCCTCCCGAGTAGCTGGGATTACAGCGGCCCGACATCACACCTGGCTAATTTTTTATATTTTTAGTA
GAGACTGGGTTTCACCATGTTAGCCAGGCTGGTCTCGAACTCCGGACCTCGGGTGATCCACCTGCCTCGGCCTCC
CAAAGTGCTGGGATCACAGGCGCGTGCCACCAGACACAGCTAATTTTTGCATTTTTAGTAGAGATTGGGTTTCTC
CATGTTGGCCAGGCTGGTCTCGAACTCCTGACCTCATGTGATCCACCCGCCTCGGCCTCCCAAAATGCTGGGGTG
ACAGGCGTGAGCCACCGCGCCCGGTCCCGATTCGAGTTCTCTTTCATGTTTGTGAACCCAGGTGGGAAGCCTTGG
GCAGGTGCGGAGAATCTGACCTGCTGGATTCATGACGTGGATTTCTTGAGCTGCAGCTGGGCGGTAGGCCCGGGG
GCCCCCGCGGACGTCCAGTACGACCTGTACTTGAACGTTGCCAAGTAGGTGTGCCCGTGGGCAGAGGCCGGGCTG
TCCCTGGTGCGGGTGCCATCGGCGTGGGGTCGTCCCCCAACCTTACCGCTTACCGCAGCAGGCGTCAACAGTACG
AGTGTCTTCACTACAAAACGGATGCTCAGGGAACACGTATCGGGTGTCGTTTCGATGACATCTCTCGACTCTCCA
GCGGTTCTCAAAGTTCCCACATCCTGGTGCGGGGCAGGAGCGCAGCCTTCGGTATCCCCTGCACAGATAAGTTTG
TCGTCTTTTCACAGATTGGTGAGTAGCCCGGGACACTCCCTCCCACCCTCAGTTCTGTGATACCACGGCTTTAGC
GCCAGGCCAGATCCCACGGGACCACGTGGCTCCCAACGCAGACGTTGGCCTCTCACATTTCCAGAGGCTGGACGT
TGGAGGTCAGCGTGCTGGCTGGCTGGGCTCCTCGGGAGGTCTCTTCCTGGCTTGGAGAGAGGGTCATCTTCTCAC
TGTGTCTTCACGTGGTGTAGAGAGAGAGAGAGATGAAGTTCTGGGGTCTCTTGTTAGAAGGGCACTAACACCATC
ATGGGTCCCATCATGGGTCATAGGATCCCTCACCATGGGTCATGGGACCCCCCATCATGGGTTCCATCATGGGTC
CCATCATGGGTCATAGAATCCCCCATGATGGGTTTCATCATGAGTCATAGAACCCCCTATCATGGATTCCATCAT
GGGTCATGGGAACCCCCATCATGGGTCCCATCATGGTTCATAGGATCCCCCATCATGGGTTTCATCATGAGTCAT
AGAACCCCCTATCATGGATTCCATCATGGGTCATGGGAACCCCCATCATGGGTCCCATCATTGGTCATAGGACCC
CCTATCATGGGTTCCATCATGGGTCATGGGACACCCCCCATCATGGGTTCCACCATGAGTCATGGGATCCCTCAT
CGTGGGTTCCATCATGGGTCATGGGACCCCCCATCATGGGTTCCATCATGGGTAATAGGACCCCCCCATCATGGG
TTCCACCATGAGTCATGGGATCCCTTATCATGGATTCCATCATGGGTCATGGGACCCCCTATCATGGATTCCATC
ATGGGTCATGGGACCCCCCCATCATGGGTCCCATCATGAGTCATGGGATCCCTCATCATGGGTTTCATCATGGGT
CATAGGACCCCCATCATGGATTCCATCATGGGTCATGGGAACCTCCATCATGGGTCCCATTATGGGTCATAGAAT
TCCCCATCATGGGTTCCATCATGAGTCATGGGACCCCCCATCATGGGTTCCACATGGGATCCCTCATCATTGGTT
CCATCATGGGTCATAGGATCCCCCATCATAGGTCCCATCATGGGTTCCACATGGGATCCCCCATCATAGGTCCCA
TCATGGGTCATGGGACCCCCCCCATCATGGGTTCCATCACGGGTCATGGGACCCCCCCCATCATGGGTTCCATCA
CGGGTCATGGGACCCCCCCCAATCATGGGTTCCATCATGGGTCATGGGAACCCCCATCATGGGTTTCATCATGGG
TCATAGGATCCCCTATCATGGGTTCCATCATGGGTCATGGGACCCCCCCCATCACGGGTTCCATCATGGGTCATG
GGAACCCCCATCATGGGTTTCATCATGGGTCATAGGATCCCCTATCATGGATTCCATCATGAGTCATGGGATCCC
TCATCATGGGTTCCATCATGGGTCATGGGACCCCCCCCCCCATCATGGGTCGTGGGACCCCCACCCCCATCATGG
GTCATGGGAGCCCCCATTGTGAGTCATGGAATCCCTCATCATGGGTCTCATCATGGGCCATGGGTCCCACCATGG
GTCATGGGAGCCCCCATCATGAGTCATGGAATCCCTCATCATGGGTCATGGGTCCCATCATGGGTCATGGGAGCC
CCCATCATGGGTCATGGGTCCCATCATGGGTCACGGGAGCCCCCATCATGGGGGTCCACCCTCATAACTTCAGCC
CACGCCAGTCACCTCCCAAAGACCCCACCTTCTAACACCGCCCAACCAGGGGTTAGAGCTTCAGTGGAGGAATTT
GGGACAGAAGGACACACACTTTCAGTCCACGATACCCGAGCTCCGAGGAACCTCCCAGGTGGTGAGAATGTCAAT
ATGCCCAGAGCTGACGTGCCCTGAACCCAAGGGCAGGGTGCTCGGATGCTTCAGAAGAGGAGGGGGAAACGAGGA
AGAGGAGGAGAAGGACAAGGAGAAGAGAAGGGAGGAGAGGAAGAACATGAGCAGGGGGAGGAGGAGGAGAAGGAA
AAGAAACAGAGGAAAAGGAGGGGGAGGAGGTAGAGATGGAGAGGGAAGGAAGAGGAGGAAGAGAAGAAAACGGAG
AAAGAGGAGGGGGAGGAGGAGGAGGAAGAGAAAATGGAGAAAGAGGAGGGGGAGGAGGTGGAGATGGGGAGGGGA
GGAAGAGGAGGAAGAGAAGAAAACAGGAGGGGGAGGAAGAGAAGAAAATGGAGGGAAAGGAGGGGGAGGAGGTGG
AGATGGGGAGAGAAGGAGCAGGAGGAGGAGAAGAAAGAAGAGGAAGAAAGGAGGAGGAGAGGGTGGAGGAGGAAG
AGGGTGAAGGAGAGAGGGAGGAGAATGGAGAGGAGGAGGAGGCGGGGGAGGGAAGAGAAGGAGGTGAGGAGGAGG
AAGGGGAGGATAGAGAAGGTGGGGTAGAAGGGTGGTGAGGTGGGGAGGGAGAAGGAGGGGGAGGAGGAGAGAGGA
GGACTGGGGGGAGGAAGGGGGAGGAGGGGAGAGAGGAGGAGGGAGGAGGGAGAAGGAGGGGGAGAGGATGGAGGA
AGGGGAGCAGATGGAGGAAGGCGAGCAGGAGGGGGAGGAAAAAGGAGGGGCAGGAGGAAGAAGGAGCGGGAGGAG
AGGAGGAGGAGGAGGAGGAGAATGGGCAGGGAGGAGGAGAGGGAAGAAAAGGAGGGGGAGGAGGAAGGAGGGGGA
GGAGGGTGGAGGGGGAGGAGGGTGGAGGGGGAGGAGGGTGGAGGGGGAGGAGGAAGGAGGGAGAAGGAGGGGAGA
GGATGGAGGAAGAGGAGCAGGAGGGGGAGGAAAAAGGAGGAGGGGCAGGAGGGGAGGAGGAGAAAGGAGAGTGGA
GGAGGAGGGTGACAAGGAGAATGGGGAGGAGAGAGGAGTGGTAGAAAAAGGAGAGAGAGGAAGAGTAGGAGCGGA
GGGGGAGGAGGGGAGGGGAGGGGAGGGGAGAAAGACCAGGAGGGTAGGAGGAGGGGGAGGAGGAGGAGGGGGCCA
GGGCTGGACTTCCTTCCAGGGGCCCAGGGGAGACCCAGCCAGGCAGCCGGACACACAGGGCTGGGCTGGTAGTCG
GGTCACACCTGAGGATGTGGAAGTGCCTGGGGTTCCCGGGAAGTGGAGGATGTCCTGGGCCACTGGGAGAGGCGG
CACATCCCCTGGGGGGCGGAGGTGGGGGAGGTGAGAAGCCAGGCAGGGGCCAGGAAGTGGAAGCTTCAGGAGGAT
GTGTGCAGGTCAAAGTGTAGGAGTCTGCATCTTGAATGATGCAGAGAGGGCCTCCAAGGCTCCAGTGTGCTGCAG
GTGGGCAGAGGGGGCATGGGAAGTAGGGGTTGGCCCTGGGCAGGGGTGGGGAGTGGGCCAGGCTGCCCAGCGGGG
CTGAGCCTAGAGATGGAAGGGGCAGGGACAAAGATGTGCAGCTGCCAGTCCTTGGAAAAGCTGAACGCCTGGTAC
TTCAGAGAACAAAAGGGTGGTTCTGAAGGCTGCTCCCAGGACAGTGGGGCTCCGAGGGTGCAACCCCAAGGCTCA
CTCCTCCCAGTGCCCCCAACGCGGCTCAGTCCTGTGTCTCTGCCTGTAGAGTTTCTTTGTTCCCTCTTCCTTCCT
GGTGTTTTTCTCTCCCGCTCTCCAAATGCATAGGAGAAGTAATTTGAAGTATCTCCAGAAAAAAAAAGAGAAAAA
GAAAAAGAATTGATTTCTTGTACTCCTAAATCCTAAAAGTGTTTTTCTCGTTGCTAGAGATATTAACTCCACCCA
ACATGACTGCAAAGTGTAATAAGACACATTCCTTTATGCACTGGAAAATGAGAAGTCATTTCAATCGCAAATTTC
GCTATGAGCTTCAGATACAAAAGGTAAACTTTCACCCCGCCCCCAGCCCCCCCACCCCCGTGGACATCCCTTATT
TTTGGTAAGTCGCACTCTGGGGCCTTGAAACGGGCAACAATCTCCTCTGATAACGTCACAGAAGGCATGGATCAT
TAAAAAACAAAAACAAAAACAAAAGGCCGGGCGCTGTGGCTCACGCCTGTCATCCCAGCACTTTGGGAGGCTGAG
GCAGGTGGATCACAAGGTCAGGAGATCGAGACCATCCTGGCTAACGCGGTGAAACCCCGTCTCTACTAAAAATAC
GAAAAAAAATTAGCTGGGCGTGGTGGCACGGTCTTGTAGTTCCAGCTACTGGGGAGGCCGAGGCAGGAGAATTGC
TTGAACCCAGGAGGTGGGGGTTGCAGTGAGCCGAGATCGCGCCATTGCACTCCAGCCTGGGCGACAGAGCGAGAC
TCCGTCTCAAAAAAAAAAAAAAAGTTTGAGACTGTATGTGGTCTGTTGTTTTTTATTTTTATTATTTCTATTGTT
ATGTGGTTTTTTAATTTTTTCTTGAACTTTCTTTTTTCTTTTGTAGTGATCTACAGATTCAATGCAAGCTTCCCA
GATATTTTTGATCCACAGTTCGTTGGATCAGTTGAACATAAATATTGATTTATTTATTGAGCCACTCTGGCTCTG
TAGCCCAGACTGGAGTGCAGTGGCTTAAATCTTGACTCACTGCAACCTCTGCCTCCCGGGTTCAAGTGATTCTCC
TGCCTCAGCCTCCCGAGTAGCTGGGATTATGGGCACCCATCACCACACCCAGCTAAGTTTTTGTATTTTTAGTAG
AAACAGAGTTTCATCATGTTGGCCAGGCTGGTCTCAAACTCCTGACCTCAGGTGATCCACCCGCCTCGGCCTCCC
AAAGTGCTGGGATTACAGGTGTGAGCCTTCGTGCGCAGCCTATGTGTTATATTTAGTTTGTATTTTATTTTATTG
TATTTTATTATTTATTTATTTATTTTTGAGACGGAGTCCTGCTCTGTCACCCAAGCTGTTTGTTTGTTTGTTTGT
TTGTTTATTATTTTTGAGACGGAGTCCTGCTCTGTTGCCCAGGCTGGGGTGCAGTGGCATGATCTCAGCTCACTG
TAACCTCCGCCTCTCGGGTTCAACCAATTCTCAGCTTCAGCCTCCTGAGTAGCTGGAATTACAGGCACCTGCCAC
CATGCCTGGCTAATTTTTTTTTGTATTTTTAGTAGAGATGGGGTTTCACCATCTTGTCCTGGCTGGTCTTGAACT
CCTGACCTCATGATCCACCCGCCTCAGCTTCCCAAAGTGCTGGGATTACAGGCGTGAGGCACTGTGCCCCGCCAT
ATATTTATTATTTATGCTCAAATACTAATTATTTCATATGCAATTTTTCTGTAAGTCTAAATCTGCTAAAAAACG
TTAGGTCTATTAATTTCTTTTATATTACCAAGTGTTTTTTAGCCAATCTGTTTGGGTTTTTTTTTTTTTTAAGAA
AATAAATGGCTGGGTGCAGTAGCTTACGCCTGTAATCCCAGCACTTTGGGAGGCCGAGGCAGGACGATCACGAGG
TCAGGAGATAGAGACCATCCTGGCTAACATGGTAAAACCCCGTCTCTACTAAAAATACAAAAAAAAATTAGCTGG
GCGTGGTGGCGGGCGCCTGTAGTCCCAGCTACTTGGGAGGCTGAGGCAGGAGAATGGCGTAAACCCAGAAGGCGG
AGCTTGCAGCGAGCTGAGATTGTGCCACTGCACTCCATCCTGGGCAACAGAGTGAGACTCCGTCTCAGGAGAAAA
AAAAAAAAAGAAAATAAATACAGCACAGACTTCTTTCTTTCACTGATTTGAGGGAGCAGGCATAGCTGCAGCCAC
AGGCAGAGTCGTAGCTAGTCTGATGTTGCACCCCCTACCTAGTTCGCTGGCCTGGCATAGGCTGTCTGTGGCTAC
CCCTGAGTGCATCTGGACACAGTCTGGGAGGACGGTGGGTCTTGTTTGTCCACCGGCCTCACAAAGCCCCCTCCC
ACCAAGGTCTTGCCACAGTGGCCCATAAGAAACCTTTTGGGCCAGGCGCGATGGGGCATGCCTGTAATCCCAGCA
CTTTGGGAGGCCGAGGTGGGCAGATCATGAGGTCAAGAGATCAAGACCATCCTGGCCAACATGGTGAAACTTTGT
TTCCACTAAAAATACAAAAAATTAGCCAGGCGTGGTGGCACGCACCTGTAGTCCCAGCTACTCGGGAGACTGAGG
CAAAAGAATCACTTGAATCCGGGAGGCGGAGGTTGCAGTGAGCCGAGATCACGCCACTGCATTCCAGCCTGGGTG
ACGGAGTGAGACTCCGTCTCATAAATAAATAAACAAACAAACCTTTTGGTCAGGTGCTATTTACTCCTAAGCTCA
TTATTTTGCCCCCACTGCTGCCCGAAGGCCTTCCCAGAGCCCTCACTGTTTTGCTGGTTTTCCTGGAGGGAGAAA
TTTGAGTTTGGGAGGAGGAGGCTTTCAGGGACGGTCCAGACACTCAAAAGTTTGCTTGCTTTTGTGTTGCAGAGA
ATGCAGCCTGTAATCACAGAACAGGTGAGTGTTCCCTACCCCCAGCCGCTGTACTTGACATTGCAAAGGGTGAGT
TTTATTATTATTAAGAATAAAATGATAAAAAATATTAATAATTCTTATTAATAAAATAATGAAAATATTATTAAT
AATAAATGTTATTATTCAATGTTCAGTGACTTTCATTGGACAGACTCTTGAGTGTCACCCTTACTGCGATCTTGC
AAAATTGGGATATTTCACATCCCCAAATTGAGGGATGGGAAAAGGAAGAGTCAGGGATGACACCTCCCAAGGTGT
GAGAGCCAGATGCTATGGCTGGCCAGGTGCTGTCCAAACGAGGTCCACCCATTTGCCCCAGATTCCTTACCCTGG
GCCAGGCAGCCCCAGTCCAGCAGGAACAAGCTACCAAACCATAGCTCCACCCAGCAGAGACAAGCATTCAGACAG
GTGGCCCAGACCTCAGACAGAGGACCCTCCCCCAGCCCCTGCCTGGGATCTGCAGGAACAGACAGCCGGACCACA
GCATTCTCTGTCCCCTCTCTCTCTGTCCATCTCTTTCCCCGTATCTCTTTCTCTGTCTCTGAATCTCTCTGCCTT
TCTCCCTCCCTCTCTTTCTCTCTCTCTCGTCTCTATCTCCCTCCCCCTCTCTGTATCTCCCTCCCTTTTTCCCTC
CCTCTATCTTTCTGGCTCTCTCCCTGTGTCTATCTCTCCCTGTCCCCATCTCTCTCTGTGTCTCTTTCCCTCCCT
CTCTCATTCTCCTTCCCTCTCTCTATCTTTCTCTCCCTGTCTCTATCTCCCGCTCCGTGTCTGTCTCTATCTTTC
TCCCTTTCTCCCTCCATCTCTCTCTCCCTGTCTCTCTCTCCCCGTTCCCATCTCTCTCTCTGTGTCTCTGTCTCT
CCCTCTCTCATTCTCCCTCCCTCTTTTATTCTCCCTCCCTCTCTTTCTCTCTCTCTCTCTCTCTCCTGGTCTCTG
TCTCCCCCCTCCCCATCTCTTTCTCTGTGTCTGTCTCTGTAACCTTCTCCATTTCTCCCTCCATCTCTCTGTCTC
TCCCTGTGTCTATCTCCCACTGTCCCTGTCTGTTTCTCCGTGTCTGTCTCTGTATCTCTCTCCCTTTCTCCCTCC
ATCTTTCTCTCTCTCCCCCGGTCTCTATCTTCCCTTCTCCCCATCTCTCTCTCTCTGTCTGTCTCTGTATCTCTC
TCCCTTTCTCCCTCCATCTTTCTCTCTCTCTCCCGGTCTCTATCTCCCCCTCTCCCCGTCTCTTTCTCCGTGTCT
GTCTCTGTGTCTCTCTCCCTTTCTCCCTCCCTCCATCTTTCTCTCTCTCTCCCGGTCTCTATTTCCTCCCTCCCC
ATCTCTTTCTCTGTGTCTGTCTCTGTATCTCTCTCCCTTTCTCCCTCCATCTTTCTCTCTCTCTCCTGGTCTCTA
TCTCCCCCTCTCCCTGTCTCTCTGTGTCTGTCTCTGTATCTCTCTCCCTTTCTCCCTCCCTCTCTTTATCTCTCT
CTCTCTCTCTGCCTCTATCTCCCACTGTCCAAATCTGTGTCTCTGTCTCTCCCTCTTTCATTCTCCCTCTCTTTC
TCTATCTCTCTCCTTGTCTATATCTCCCCCTCTCCCAGTCTCTCTGTATCTCTGTATCTCTCTCCCTTTCTCCTT
CCCTCTCTTCATCTTTCTTTTTGAAACGGAGTTTTTCTCTTGTTGCCCAGGCTGGAGTGCGATGGCACGATCTCG
GCTCACTGCAACCTCCGCCTCCCAGGTTCAAGCGAATCTACTCCCTCAGCCTCCCAAGTAGCTGGGATTACAGGC
ACTCGCCACCATGCCCAACTAACTTTTTTTTTTTTTTGTATTTTTAGTAGAGACAGGGTTTCACTAGTGGGCCAG
GCTGGTCTCGAACTCCTGACCTCAGGCGATCCACCTGCCTCATCCTCTCAAAGTGCTGGAATTACAGGTGTGAGC
CACCGTGCCCGGCCCCTCTCTTTATCTTTCTAGCTCTCTCCCTGTCTCTCTCTCCCTTCCCCTCTCTGTCTCTCT
CTCCCTTCCCCTCTCTGTCTCTCTCTCCCTTCCCCTCTCTGTCTCTCTCTCCCTTCCCCTCTGTCTCTCTCTCCC
TTCCCCTCTCTGTCTCTCTCTCCCTTCCCCTCTCTGTCTCTCTCTCCCTTCCCCTCTGTCTCTCTCTCCCTTCCC
CTCTCTGTCTCTCTCTCCCTTCCCCTCTCTGTCTCTCTCTCCCTTCCCCTCTCTGTCTCTCTCTCCCTTCCCCTC
TCTGTCTCTCTCTCCCTTCCCCTCTCTGTCTCTCTCTCCCTTTCCTTCTCTGTCTCTCTCTCCCTTCCCCTCTCT
CTCTCTCCATTCCCCTGTCTCTATGTCTCTCCCTTTCTCTCTCCCATTATTTCTCTGTGATTGTCTCTTTCTTTC
TCTGCCTCTGTCTGTCTGTCCCCCTGTATTAGTCCATTGTCACACTGCTGATAAACATATACCCAAGACTGGGTA
ATTTATAAACAAAAGAGGGTTCCTGGACTCACAGTTCCATGTGGCTGGGGAGGCCTCACGATCACGGCAGAAGGT
GAAGGAGGAGGAAAGGCATGTCTTACATGTCAGCGGGCAAGACAGAATGAGACAGTCGCCGGGCGCAGTGACTCA
TGCCTGTAATCACAGCACCTTGGGAGGACGAGGTGGGTGGACCACGAGGTCAGGAGTTCAAGACCAGCCTGGCCA
ACATGGTGAAACCTAGCTCTACTAAAAATAAAAAAAATTACCCAGACGTGGTGGTGGCAGGCGCCTGTGGTCCCA
GCTACTCGGGAGGCTGAAGCAGGAGAATCGCTTGAACCCGGGAGGCAGAGGTTGCAGTGAGTTGAGATCGCGCCA
CTGCACTCCAGCCTGGGTGACAGAGTGAGACTCCGTCTCGAGAAAAAAAAAAAAAAAAAAAAAATGAGAGCCGAG
AGAAAGGTTTCCCTTATTAAACCATTAGATCTCGTGAGACTTATTCACTACCCCGAGAACAGTGTGGGGGGAAAC
TGCCCCCATGATTCAGTTATGTCCTACCCAGTCCCTCTCACAACACGTGGGAGTTATAAGGGCTACAATTCAAGA
TGAGATTTGGGTGGGGACACAGCCAAACTACATCACCCACTCTCTCTGTCTCTCTGCTTCTGTTTTCCTCTCTGT
CTCTGTTTTTCTTTCCCTCTCTCTGTCTCTTTGTATCTCTGTCTCTCTCTCTCTGTCTCCGTTTCTATCTCTGTC
TCTCTCTGTCCATCACCCACTCTGTCTCTTTGTCTCTCTATGTCTCTCTGTTTCTGTTTTTCTGTTTTTCTCTTT
CCCTCTCTCTTTGTATCTATGTCTTTCTGTCTCTCCGTTTCTGTCTCTCTCTCTGTCCATCACCCACTCTGTCTG
TCTTTGTCTATGTCTCTCTGTTTTTCTGTCTCTTTTTCTCTTTCCCTCTCTGTCTCCCCTCTCTGTGTCTCTTTG
TATCTCCGTCTCTCCCTGTCTGTTTCTATCTCTGTCTCTCTCTGTCCATCACCTACTCTGTCTCTTTGTCTCTGT
CTGTTTCTGTTTTTCTTTCTTTTTCCCTCTGTCTCCCTTCTCTGTGTCTCTTTGTATCTCTGTCTCTCTCTGTTT
CTATCTGTCTCTCTCTGTCCATCACCCACTATCTCTGTCTATGTCTGTTTTTCTGTCTCTGTTTTTCTCTCTCCC
TCTCTCTGTCTCCCCTCTCTGTGTCTCTTTGTATCTCTTTCTCTCTCTCTGTCTCTCCCTGTCTGTTTCTATCTC
CGTCCTCCTGTATTAGTCCATTTTCATGGACTAATGAGGATCCTTCCCGCTTCTCCCAGCTCCTGGGGACTCTGG
GGTCCCTGGGCTTGTGGCCATATCACTCCACTCTCTGCCTCCGTCTCCACATGGCCTTCTCCTCTGCATCCGTGT
CTCCTTTTTATTTATTTATTTATTTATTTTGAGACAGAGTCTCGCTCTGTCACCCAGGCTGGAGTGCAGTGGTGC
GATCTCAGCTCACTGCAACCTCCGCCTCCCAGGTTCAAGCGATTCCCCTGCCTCAGCCTCCCGAGTAGCTGGGAC
TACAGGTGCACACCACCACGCCTGGCTAATTTTTGTATTATTAGTAAAGATGGGATTTTACCTTGTTGCTCAGGT
TGACCTCGAACTCCTGACCTCAGGTGATCTACCCACCTCAGCCTCCCAAAGTGCTGGGATTACAGGTGTCAGCCA
CCGCGCCCGGCCACGTCTCCTCTTCTTATAAGGATATTGGTCATTGCATTTAGGGCCCCCCCTGATCCAAAATGA
CATCATCTCAATCTATATCTTAACGACATCTGCCATGAACAGGTATTTCATCATAAGGCCCCATTGTAAGGTGCT
GGGGGCTAAGACTGCAACATATGAATTTTGGGGGATCACAATGCAGCCTCATTCTTTTGTACCCATCAAATATGA
ATGGTCTTTTCTTCTTTTTTTTTTTTTTTTGAGACGGAGTCTCGCTCTGTCACCCAGGCTGGAGTGCAGTGGCGC
AATCTCGGCTCACTGCAAGCTCCGCCTCCCGGGTTCCCGCCACTCTCCTGCCTCAGCCTCCTGAGTAGCTGGGAC
CTCAGGCGCCCGCCACCGGGCCCGGCTAATTTTTTGTATTTTTAGTAGAGACGGGGTTTCACTGTGGTCTCGATC
TCCTGACCTTGTGATCTGCCCGCCTTGGCCCTCCAAAGTGCTGGGATTACAGGCGTGAGCCACCGCGCCTGGCCG
AGCTATTTCATCATAAGGCCCCACTGTAAGGAGCTGGGGGCTAAGACTGCAACATATGAATTTTGGGGGATCACA
ATGCAGACTCATTCTTTTGTACCCATCAAATATGAATGGTCTTTTCTTCTACTTTTTTGTTTGGTTTTTAATTTC
AGACAGGCTCTCACTCTGTCACCCACGCTAGAGTGCAGTGGCACAGTCACGGCTCACTGCAATCTCTGCCTCCTG
GGCTCAAGGGATCCTCCTGCCTCAGCCCCCCAAGTAGCTGAAACTACAGGCGTTTGCCACCAGGTCAGCTCATTT
AAAAAAAAATTTGGCTGGGCGCGGTGGCTCACGCTGGTAATCCCAGCCCTTTGGGAGGCCGAGGCGGGTGGATCA
TGAGGTCAGGAAATCAAGACCATCCTGGCTAACATGGTGAAACCCTGTCTCTACTAAAAAAAAAAATACAAAAAA
TTAGCTGGGCATGGTAGCGGGTGCCTGTAGTCCCAGCTACTCGGGAGGCTGAGGCAGGAGAATGGCATGAACCCG
GGAAGCGGAGCTTGCAGTGAGCCGAGATCACGCCACTGCACTCCAGCCTGGGGGACAGAGTGAGACTCCATCTCA
AAAAAAAAAAAATTTTTTTTTGGCTGGACGTGGTGGCTCACACTGGTAATCCCAGCACTTTGGGAGGCTGAGGCG
GGTGGATCACCGGAGGTTGGGAGTTCGAGACCAGCCTGACCAACATGGAGAAACCCCGACTCTACTAAAAATACA
AAATTAACCGAGCGTGGTGGTGGGCGCCTGTAATCCCAGCTACTCGGGAGGCTGAGGCAGGAGAATCGCTTGAAT
CTGGGAGGCGGAGGTTGCGGTGAGCCGAGTTGGTGCCACTGCACTCCAGCCTGGGCAACAAGAGTGAAACTCTGT
CTCAAAACAAAACAAAACAAATTTTCTTCTAGAGATGGGGTCTTGGTTTGTTGCCCAGGCTGGTCTCAAACTCCT
AAGCTCGAGGTATCCCCCCACCTCAGCCTCCCTGGTAGCTGGGACCAAAGACATGTGTCACCACGTCTGGTAATT
TTTACAGTTTTTTTTTTTTGGTAAAGATGGGGTCTTGCTATGTTGCCCAGGCTGGTGTCAAACTCCTGGGCTTAA
GGAATTCTCCCACCTCAGCCTCCAAAAGTGCTGTGGTGAGAGGCATAAACCGTAGCACCCAGCCCCTCTTTTCTT
CTTTTATTTATTTATTTATTTATTTATTTATTTATTTAGAGACAGAGTCTCACTCTGTTGCCCAGGCTGCAGTGC
AGTGGTGCCATCTCGCCTCACTGCAACCTCCACCTCTCAGGTTTAAGCTATTCTCCTGCCTCAGCCTCCTGAGTA
GCTGGGATTACAGGTGCCTGCCACCATGCCAGGCTAATTTTTGTATTTTTAGTAGAGACAGGGTTTCACCATGTT
GGCCAGGCTGGTCTCGAACTCCTGACCTCAGGTGATCCACCTGCCTTGGCCTCCCTAAGTGCCCAGGTGAGAGTC
ATGAGCCACCATACCTGGCCCCTCTTCTTTATTTTCTTTCAAACCACAGGTCAGAGACAGAACCTCCTTCCAGCT
ACTCAATCCTGGAACGTACACAGTACAAATAAGAGCCCGGGAAAGAGTGTATGAATTCTTGAGCGCCTGGAGCAC
CCCCCAGCGCTTCGGTGAGTGGGCTGTGCGGGGTGCGCGGGGTGAGCGGGGTGAGCGGGGTGCGCGGGGTGAGCG
GGGTGCGCGGGGTGAGCGGGGTGCGCGGGGTGAGCGGGGTGAGCGGGGTGAGCCGGGTGCGCGGGGTGAGCCGGC
TGCGCGGGGTGAGCGGGGTGAGCGGGGTGAGCGGGGTGAGCGGGGTGCGCGGGGTGAGCGGGGTGAGCGGGGTGC
GCGGGGTGAGCGGGGTGAGCCGGGTGCGCGGGGTGAGCCGGGTGAGCGGGGTGCGCGGGGTGAGCGGGGTGAGCG
GGGTGCGCGGGGTGAGCCGGGTGCGCGGGGTGAGCGGGGTGAGCCGGGTGCGCGGGGTGAGCGGGGTGAGCGGGG
TGAGCCGGGTGAGCCGGGTGAGCCGGGTGCGCGGGGTGAGCCGGGTGCGCGGGGTGAGCGGGGTGCGCGGGGTGA
GCGGGGTGCGCGGGGTGAGCCGGGTGCGCGGGGTGAGCGGGGTGCGCGGGGTGAGCGGGGTGCGCGGGGTGAGCC
GGGTGCGCGGGGTGAGCGGGGTGAGCGGGGTGAGCCGGGTGCCCCGGGTGAGCGGGGTGCGCGGGGTGAGCCGGG
TGCGCGGGGTGAGCGGGGTGAGCGGGGTGCGCGGGGTGAGCCGGGTGCGCGGGGTGAGCCGGGTGCGCGGGGTGA
GCCGGGTGAGCGGGGTGAGCGGGGTGCGGGGTGCGCGGTGCGCGGGGTGAGCGGGGTGCGCGGGGTGAGCCGGGT
GCGCGGGGTGAGCGGGGTGAGCCGGGTGCCCCGGGTGAGCGGGGTGCGCGGGGTGAGCCGGGTGCGCGGGGTGAG
CGGGGTGCGCGGGGTGAGCGGGGTGCGCGGGGTGCGCGGCGTGAGCCGGGTGCGCGGGGTGCGCGGGGTGAGCGG
GGTGCGCGGGGTGCGCGGGGTGCGCGGGGTGAGCGGGGTGCGCGGGGTGCGCGGCGTGAGCCGGGTGCGCGGGGT
GAGCGGGGTGCGCGGGGTGAGCGGGGTGCGCGGGGTGAGCCGGGTGCGCGGGGTGAGCCGGGTGAGCCGGGTGCG
CGGGGTGAGCGGGGTGCGCGGGGTGAGCCGGGTGAGCCGGGTGCGCGGGGTGAGCCGGGTGCGCGGGGTGAGCGG
GGTGCGCGGGGTGAGCCGGGTGCGCGGGGTGAGCGGGGTGCGCGGGGTGAGCGGGGTGCGCGGGGTGAGCGGGGT
GCGCGGGGTGAGCCGGGTGCGCGGGGTGAGCGGGGTGAGCGGGGTGAGCCGGGTGCCCCGGGTGAGCGGGGTGCG
CGGGGTGAGCCCGGTGAGCCGGGTGCGCGGGGTGAGCCGGGTGCGCGGGGTGAGCCGGGTGAGCGGGGTGAGCGG
GGTGCGCGGTGCGCGGGGTGAGCGGGGTGCGCGGGGTGAGCCGGGTGCGCGGGGTGAGCGGGGTGAGCCGGGTGC
CCCGGGTGAGCGGGGTGCGCGGGGTGAGCCGGGTGCGCGGGGTGAGCGGGGTGCGCGGAGTGAGCGGGGTGCGCG
GGGTGCGCGGCGTGAGCCGGGTGCGCGGGGTGCGCGGGGTGAGCGGGGTGCGCGGGGTGCGCGGGGTGCGCGGGG
TGAGCGGGGTGCGCGGGGTGCGCGGCGTGAGCCGGGTGCGCGGGGTGAGCGGGGTGCGCGGGGTGAGCGGGGTGC
GCGGGGTGAGCCGGGTGCGCGGGGTGAGCGGGGTGCGCGGGGTGAGCCGGGTGCGCGGGGTGCGCGGGGTGCGCG
GGGTGCGCCGGGTGCGCGGGGTGAGCCGGGTGCGCGGGGTGAGCGGGGTGCGCGGGGTGCGCGGGGTGCGCGGGG
TGAGCCGGGTGCGCGGGGTGAGCGGGGTGAGCCGGGTGCGCGGGGTGAGCGGGGTGAGCCGGGTGCGCGGGGTGA
GCGGGGTGCGCGGGGTGAGCCGGGTGCGCGGGGTGAGCGGGGTGCGCGGGGTGAGCCGGGTGCGCGGGGTGAGCG
GGGTGCGCCGGGTGAGCGGGGTGAGCGGGGTGAGCCGGGTGCGCGGGGTGCGCGGGGTGCGCCGGGTGCGCGGGG
TGAGCCGGGTGCGCGGGGTGAGCGGGGTGCGCGGGGTGCGCGGGGTGCGCGGGGTGAGCCGGGTGCGCGGGGTGA
GCGGGGTGAGCCGGGTGAGCGGGGTGCGCGGGGTGAGCCGGGTGCGCGGGGTGAGCGGGGTGCGCGGGGTGAGCC
GGGTGCGCGGGGTGCGCGGGGTGAGCCGGGTGCGCGGGGTGAGCGGGGTGCGCGGGGTGAGCGGGGTGCGCGGGG
TGAGCCGGGTGCGCGGGGTGAGCCGGGTGCGCGGGGTGCGCGGGGTGCGCGGCGTGAGCCGGGTGCGCGGGGTGC
GCCGGGTGCGCGGGGTGAGCGGGGTGAGCCGGGTGCGCGGGGTGAGCGGGGTGCGCGGGGTGAGCGGGGTGCGCC
GGGTGAGCCGGGTGCGCGGGGTGAGCCGGGTGCGCGGGGTGAGCGGGGTGCGCGGGGTGAGCCGGGTGCGCGGGG
TGAGCGGGGTGCGCCGGGTGAGCGGGGTGCGCGGGGTGAGCGGGGTGCGCCGGGTGAGCGGGGTGCGCGGGCTGA
GCGGGGTGCGCCGGGTGAGCGGGGTGCGCGGGGTGAGCGGGGTGCGCCATCCTGGGTCACGGAAACACTCCTCTC
CTGCAAAGGAGAGGAGATTCACTCCCCCAGTTTCTGTGACCCCAAAAAGGACCCTGAACCCGACGGTGAACTCAC
AGCTTGCTCTTACTCACGAGAGGAGACGTGGAGGGGAAACAAGGTCGTCCCACTGACAGACACCCCCTGGGCCTT
GTAATAAAGACCGAGGCGGGCGGATCACAAGGTCAGGAGATCGAGACCATCCCGGCTAACACGGTGAAACCCGGT
CTCTACAAAAAATGTAACAAATTAGCCAGGCGTGGTGATGGGCGCCTGTAGTCCCAGCTACTCGGGAGGCTGAGG
CAGGAGAATGGCGTGAACCCGGGAGGCGGAGGTTGCAGTGAGCCGAGATCGCGCCACTGTGCTCCATCCAGCCTG
GGCGACAAGAGCGAGACTCCATCTCAACAACAAAACAAAAGGATCGCCTCAGAGTAGAACTTCTGGCCGGGCACG
GTGACTCATGCCTGCCATCCCAGCACTTTGGGAGGCTGAGGTGGGTAGATCACCTGAGGTCAGGAGTTCGAGACC
AGCCTGACCAACATGGAAAAACCCTGTCTCTACTAAAAATAGAAAAATTAGGTCAGGTGCTGTGGCTTAAGCCTG
TAACCTCAGCTACCAGGGAGGCTGAGGCAGGAGAATCGCTTGAATTGTGTTCCCTCAAAATTCGTGTGTTGAAGC
TTTGATCCCCCAGGACCTCAGAATGTGACTGTGTTTGGAGTTGGGGTGTTTAAAGAGGCGATTAAGGTAAAATGA
GGTCATTAGGGTGGGCCCTAATCTAACAGGACTGGGGTCCTTATAAGAAGAGGAGATGAGGACACAGACACACAC
AGAGGGACGACCCTGTGGGACACAGGGAGAAGACGGCGTCTCCAAGCCCAGGAGAGGGGCCTCAGGAGGAACCAG
CACTGCCCACACCTGGATCTCAGACCTCCAGCCTCCAGGGCTGTGGGAGAATCAATGTGTTTTGTTTCTAAGCCG
CCCAGCCTCTGGTATTCTGTGACAGCAGCCTGAGATGGACTAAGCCATCTCATAAGAAGAGACGAGGACACAGAC
ACACACAGAGGGACGACCCTGTGAGGGCACAGGGAGAAGACGGCGTCTCCAAGCCCAGGAGAGGGGCCTCAGGAG
GAACCAGCACTGCCCACACCTGGATCTCAGACCTCCAGCCTCCAGGGCTGTGGGAGAATCAATGTGTTTTGTTTC
TAAGCCGCCCAGCCTCTGGTATTCTGTGACAGCAGCCTGAGATGGACTAAGACATCTCATAAGAAGAGGAGATGA
GGACACAGACACACACAGAGGGACGACCCTGTGAGGGCACAGGGAGAAGACGGCGTCTCCAAGCCCAGGAGAGGG
GCCTCAGGAGGAACCAGCCCTGCCCACACCTGGATCTCAGACCTCCAGCCTCCAGGGCTGTGGGAGAATCAATGT
GTTTTGTTTCTAAGCCGCCCAGCCTCTGGTATTCTGTGACAGCAGCCTGAGATGGACTAAGACATCTCATAAGAA
GAGGAGATGAGGACACAGACACACACAGAGGGACGACCCTGTGAGGGCACAGGGAGAAGACGGCGTCTCCAAGCC
CAGGAGAGGGGCCTCAGGAGGAACCAGCACTGCCCACACCTGGATCTCAGACCTCCAGCCTCCAGGGCTGTGGGA
GAATCAATGTGTTTTGTTTCTAAGCCGCCCAGCCTCTGGTATTCTGTGACAGCAGCCTGAGATGGACTAAGCCAT
CTCATAAGAAGAGACGAGGACACAGACACACACAGAGGGATGACCCTGTGAGGGCACAGGGAGAAGACGGCGTCT
CCAAGCCCAGGAGAGGGGCCTCAGGAGGAACCAGCACTGCCCACACCTGGATCTCAGACCTCCAGCCTCCAGGGC
TGTGGGAGAATCAATGTGTTTTGTTTCTAAGCCGCCCAGCCTCTGGTATTCTGTGACAGCAGCCTGAGATGGACT
AAGACATCTCATAAGAAGAGGAGATGAGGACACAGACACACACAGAGGGATGACCCTGTGAGGGCACAGGGAGAA
GACGGCGTCTCCAAGCCCAGGAGAGGGGCCTCAGGAGGAACCAGCCCTGCCCACACCTGGATCTCAGACCTCCAG
CCTCCAGGGCTGTGGGAGAATCAATGTGTTTTGTTTCTAAGCCGCCCAGCCTCTGGTATTCTGTGACAGCAGCCT
GAGATGGACTAAGCCATCTCATAAGAAGAGACGAGGACACAGACACACACAGAGGGACGACCCTGTGAGGACACA
GGGAGAAGACGGCGTCTCCAAGCCCAGGAGAGGGGCCTCAGGAGGAACCAGCACTGCCCACACCTGGATCTCAGA
CCTCCAGCCTCCAGGGCTGTGGGAGAATCAGTGTTTTGTTTCTAAGCCGCCCAGCCTCTGGTATTCTGTGACAGC
AGCCTGAGATGGACTAAGCCATCTCATAAGAAGAGACGAGGACACAGACACACACAGAGGGACGACCCTGTGAGG
GCACAGGGAGAAGACGGCGTCTCCAAGCCCAGGAGAGGGGCCTCAGGAGGAACCAGCCCTGCCCACACCTGGATC
TCAGACCTCCAGCCTCCAGGGCTGTGGGAGAATCAATGTGTTTTGTTTCTAAGCCGCCCAGCCTCTGGTATTCTG
TGACAGCAGCCTGAGATGGAGTAAGACATCTCATAAGAAGAGGAGATGAGGACACAGACACACACAGAGGGACGA
CCCTGTGGGACACAGGGAGAAGACGGGGTCTCCAAGCCCAGGAGAGGGGCCTCAGGAGGAACCAGCCCTGCCCAC
ACCTGGATCTCAGACCTCCAGCCTCCAGGGCTGTGGGAGAATCAATGTGTTTTGTTTCTAAGCCGCCCAGCCTCT
GGTATTCTGTGACAGCAGCCTGAGATGGACTAAGCCATCTCATAAGAAGAGACGAGGACACAGACACACAGAGAG
GGACGACCCTGTGAGGACACAGGGAGAAGACGGCATCTCCAAGCCCAGGAGAGGGGCCTCAGGAGGAACCAGCAC
TGCCCACACCTGGATCTCAGACCTCCAGCCTCCAGGGCTGTGGGAGAATCAATGTGTTTTGTTTCTAAGCCGCCC
AGCCTCTGGTATTCTGTGACAGCAGCCTGAGATGGACTAAGACATCTCATAAGAAGAGGAGATGAGGACACAGAC
ACACACAGAGGGACGACCCTGTGAGGACACAGGGAGAAGACGGCGTCTCCAAGCCCAGGAGAGGGGCCTCAGGAG
GAACCAGCCCTGCCCACACCTGGATCTCAGACCTCCAGCCTCCAGGGCTGTGGGAGAATCAATGTGTTTTGTTTC
TAAGCCGCCCAGCCTCTGGTATTCTGTGACAGCAGCCTGAGATGGACTAAGCCATCTCATAAGAAGAGACGAGGA
CACAGACACACACAGAGGGACGACCCTGTGAGGGCACAGGGAGAAGACGGCGTCTCCAAGCCCAGGAGAGGGGCC
TCAGGAGGAACCAGCCCTGCCCACACCTGGATCTCAGACCTCCAGCCTCCAGGGCTGTGGGAGAATCAATGTGTT
TTGTTTCTAAGCCGCCCAGCCTCTGGTATTCTGTGACAGCAGCCTGAGATGGACTAAGCCATCTCATAAGAAGAG
ACGAGGACACAGACACACACAGAGGGACGACCCTGTGAGGGCACAGGGAGAAGACGGCGTCTCCAAGCCCAGGAG
AGGGGCCTCAGGAGGAACCAGCCCTGCCCACACCTGGATCTCAGAACTCCAGCCTCCAGGGCTGTGGGAGAATCA
ATGTGTTTTGTTTCTAAGCCGCCCAGCCTCTGGTATTCTGTGACAGCAGCCTGAGATGGACTAAGACATCTCATA
AGAAGAGGACATGAGGACACAGACACACACAGAGGGACGACCCTGTGGGACACAGGGAGAAGACGGGGTCTCCAA
GCCCAGGAGAGGGGCCTCAGGAGGAACCAGCCCTGCCCACACCTGGATCTCAGACCTCCAGCCTCCAGGGCTGTG
GGAGAATCAATGTGTTTTGTTTCTAAGCCGCCCAGCCTCTGGTATTCTGTGACAGCAGCCTGAGATGGACTAAGA
CATCTCATAAGAAGAGGAGATGAGGACACAGACACACACAGAGGGACGACCCTGTGGGACACAGGGAGAAGACGG
GGTCTCCAAGCCCAGGAGAGGGGCCTCAGGAGGAACCAGCCCTGCCCACACCTGGATCTCAGAACTCCAGCCTCC
AGGGCTGTGGGAGAATCAATGTGTTTTGTTTCTAAGCCGCCCAGCCTCTGGTATTCTGTGACAGCAGCCTGAGAT
GGACTAAGACATCTCATAAGAAGAGGAGATGAGGACACAGACACACACAGAGGGACGACCCTGTGAGGACACAGG
GAGAAGACGGCGTCTCCAAGCCCAGGAGAGGGGCCTCAGGAGGAACCAGCCCTGCCCACACCTGGATCTCAGACC
TCCAGCCTCCAGGGCTGTGGGAGAATCAATGTGTTTTGTTTCTAAGCCGCCCAGCCTCTGGTATTCTGTGACAGC
AGCCTGAGATGGACTAAGCCATCTCATAAGAAGAGACGAGGACACAGACACACACAGAGGGACGACCCTGTGAGG
GCACAGGGAGAAGACGGCGTCTCCAAGCCCAGGAGAGGGGCCTCAGGAGGAACCAGCCCTGCCCACACCTGGATC
TCAGACCTCCAGCCTCCAGGGCTGTGGGAGAATCAATGTGTTTTGTTTCTAAGCCGCCCAGCCTCTGGTATTCTG
TGACAGCAGCCTGAGATGGACTAAGCCATCTCATAAGAAGAGACGAGGACACAGACACACACAGAGGGACGACCC
TGTGAGGGCACAGGGAGAAGACGGCGTCTCCAAGCCCAGGAGAGGGGCCTCAGGAGGAACCAGCCCTGCCCACAC
CTGGATCTCAGAACTCCAGCCTCCAGGGCTGTGGGAGAATCAATGTGTTTTGTTTCTAAGCCGCCCAGCCTCTGG
TATTCTGTGACAGCAGCCTGAGATGGACTAAGACATCTCATAAGAAGAGGACATGAGGACACAGACACACACAGA
GGGACGACCCTGTGGGACACAGGGAGAAGACGGGGTCTCCAAGCCCAGGAGAGGGGCCTCAGGAGGAACCAGCCC
TGCCCACACCTGGATCTCAGACCTCCAGCCTCCAGGGCTGTGGGAGAATCAATGTGTTTTGTTTCTAAGCCGCCC
AGCCTCTGGTATTCTGTGACAGCAGCCTGAGATGGACTAAGACATCTCATAAGAAGAGGAGATGAGGACACAGAC
ACACACAGAGGGACGACCCTGTGGGACACAGGGAGAAGACGGGGTCTCCAAGCCCAGGAGAGGGGCCTCAGGAGG
AACCAGCCCTGCCCACACCTGGATCTCAGAACTCCAGCCTCCAGGGCTGTGGGAGAATCAATGTGTTTTGTTTCT
AAGCCGCCCAGCCTCTGGTATTCTGTGACAGCAGCCTGAGATGGACTAAGACATCTCATAAGAAGAGGACATGAG
GACACAGACACACACAGAGGGACGACCCTGTGGGACACAGAAGACGGGGTCTCCAAGCCCAGGAGAGGGGCCTCA
GGAGGAACCAGCCCTGCCCACACCTGGATCTCAGACCTCCAGCCTCCAGGGCTGTGGGAGAATCAATGTGTTTTG
TTTCTAAGCCGCCCAGCCTCTGGTATTCTGTGACAGCAGCCTGAGATGGACTAAGACATCTCATAAGAAGAGGAC
ATGAGGACACAGACACACACAGAGGGACGACCCTGTGAGGGCACAGGGAGAAGACGGCGTCTCCAAGCCCAGGAG
AGAGGCCTCAGGAGGAACCAGCCTTGCCCACACCTGGATCTCAGACCTCCAGCCTCCAGGGCTGTGGGAGAATCA
ATGTGTTTTGTTTCTAAGCCGCCCAGCCTCTGGTATTCTGTGACAGCAGCCTGAGATGGACTAAGACACCTCATA
AGAAGAGGAGATGAGGACACAGACACACACGGAGGAACAACCCTGTGAGGACACAGGGAGAAGACGGCATCTCCA
AGCCCAGGAGAGAGGCCTCAGGAGGAACCAGCCCTGCCCACACCTGGATCTCAGACCTCCAGCCTCCAGGGCTGT
GGGAGAATCAATGTGTTTTGTTTCTAAGCCGCCCAGCCTCTGGTATTCTGTGACAGCAGCCTGAAACGGACTAAG
ACATCCCATAAAAAGGAGATGAGGACACAGACACACACGGAGGGACGACCCTGTGGGACACAGGGAGAAGACGGC
GTCTCCAAGCCCAGGAGAGGGGCCTCAGGAGGAACCAGCACTGCCCACACCTGGATCTCAGACCTCCAGCCTCCA
GGGCTGTGGGAGAATCAATGTGTTTTGTTTCTAAGCCGCCCAGCCTCTGGTATTCTGTGACAGCAGCCTGAGATG
GACTAAGCCATCTCATAAGAAGAGACGAGGACACAGACACACACAGAGGGATGACCCTGTGAGGGCACAGGGAGA
AGACGGCGTCTCCAAGCCCAGGAGAGGGGCCTCAGGAGGAACCAGCACTGCCCACACCTGGATCTCAGACCTCCA
GCCTCCAGGGCTGTGGGAGAATCAATGTGTTTTGTTTCTAAGCCGCCCAGCCTCTGGTATTCTGTGACAGCAGCC
TGAGATGGACTAAGACATCTCATAAGAAGAGGAGATGAGGACACAGACACACACAGAGGGACGACCCTGTGAGGG
CACAGGGAGAAGACGGCGTCTCCAAGCCCAGGAGAGGGGCCTCAGGAGGAACCAGCCCTGCCCACACCTGGATCT
CAGACCTCCAGCCTCCAGGGCTGTGGGAGAATCAATGTGTTTTGTTTCTAAGCCGCCCAGCCTCTGGTATTCTGT
GACAGCAGCCTGAGATGGACTAAGCCATCTCATAAGAAGAGACGAGGACACAGACACACACAGAGGGACGACCCT
GTGAGGACACAGGGAGAAGACGGCGTCTCCAAGCCCAGGAGAGGGGCCTCAGGAGGAACCAGCACTGCCCACACC
TGGATCTCAGACCTCCAGCCTCCAGGGCTGTGGGAGAATCAATGTGTTTTGTTTCTAAGCCGCCCAGCCTCTGGT
ATTCTGTGACAGCAGCCTGAGATGGACTAAGCCATCTCATAAGAAGAGACGAGGACACAGACACACACAGAGGGA
CGACCCTGTGAGGGCACAGGGAGAAGACGGCGTCTCCAAGCCCAGGAGAGGGGCCTCAGGAGGAACCAGCCCTGC
CCACACCTGGATCTCAGACCTCCAGCCTCCAGGGCTGTGGGAGAATCAATGTGTTTTGTTTCTAAGCCGCCCAGC
CTCTGGTATTCTGTGACAGCAGCCTGAGATGGAGTAAGACATCTCATAAGAAGAGGAGATGAGGACACAGACACA
CACAGAGGGACGACCCTGTGGGACACAGGGAGAAGACGGGGTCTCCAAGCCCAGGAGAGGGGCCTCAGGAGGAAC
CAGCCCTGCCCACACCTGGATCTCAGACCTCCAGCCTCCAGGGCTGTGGGAGAATCAATGTGTTTTGTTTCTAAG
CCGCCCAGCCTCTGGTATTCTGTGACAGCAGCCTGAGATGGACTAAGCCATCTCATAAGAAGAGACGAGGACACA
GACACACACAGAGGGACGACCCTGTGAGGGCACAGGGAGAAGACGGCGTCTCCAAGCCCAGGAGAGGGGCCTCAG
GAGGAACCAGCCCTGCCCACACCTGGATCTCAGACCTCCAGCCTCCAGGGCTGTGGGAGAATCAATGTGTTTTGT
TTCTAAGCCGCCCAGCCTCTGGTATTCTGTGACAGCAGCCTGAGATGGACTAAGACATCTCATAAGAAGAGGACA
TGAGGACACAGACACACACAGAGGGACGACCCTGTGGGACACAGGGAGAAGACGGGGTCTCCAAGCCCAGGAGAG
GGGCCTCAGGAGGAACCAGCCCTGCCCACACCTGGATCTCAGACCTCCAGCCTCCAGGGCTGTGGGAGAATCAAT
GTGTTTTGTTTCTAAGCCGCCCAGCCTCTGGTATTCTGTGACAGCAGCCTGAGATGGACTAAGACATCTCATAAG
AAGAGGAGATGAGGACACAGACACACACAGAGGGACGACCCTGTGGGACACAGGGAGAAGACGGGGTCTCCAAGC
CCAGGAGAGGGGCCTCAGGAGGAACCAGCCCTGCCCACACCTGGATCTCAGACCTCCAGCCTCCAGGGCTGTGGG
AGAATCAATGTGTTTTGTTTCTAAGCCGCCCAGCCTCTGGTATTCTGTGACAGCAGCCTGAGATGGACTAAGACA
TCTCATAAGAAGAGGACATGAGGACACAGACACACACAGAGGGACGACCCTGTGGGACACAGAAGACGGGGTCTC
CAAGCCCAGGAGAGGGGCCTCAGGAGGAACCAGCCCTGCCCACACCTGGATCTCAGACCTCCAGCCTCCAGGGCT
GTGGGAGAATCAATGTGTTTTGTTTCCAAGCCGCCCAGCCTCTGGTATTCTGTGACAGCAGCCTGAGATGGACTA
AGACATCTCATAAGAAGAGGACATGAGGACACAGACACACACAGAGGGACGACCCTGTGAGGGCACAGGGAGAAG
ACGGCGTCTCCAAGCCCAGGAGAGAGGCCTCAGGAGGAACCAGCCTTGCCCACACCTGGATCTCAGACCTCCAGC
CTCCAGGGCTGTGGGAGAATCAATGTGTTTTGTTTCTAAGCCGCCCAGCCTCTGGTATTCTGTGACAGCAGCCTG
AGATGGACTAAGACACCTCATAAGAAGAGGAGATGAGGACACAGACACACACGGAGGAACAACCCTGTGAGGACA
CAGGGAGAAGACGGCGTCTCCAAGCCCAGGAGAGAGGCCTCAGGAGGAACCAGCCCTGCCCACACCTGGATCTCA
GACCTCCAGCCTCCAGGGCTGTGGGAGAATCAATGTGTTTTGTTTCTAAGCCGCCCAGCCTCTGGTATTCTGTGA
CAGCAGCCTGAAACGGACTAAGACATCCCATAAAAAGGAGATGAGGACACAGACACACACGGAGGGACGACCCTG
TGGGACACAGGGAGAAGACGGCGTCTCCAAGCCCAGGAGAGGGGCCTCAGGAGGAACCAGCCCTGCCCACACCTG
GATCTCAGACCTCCAGCCTCCAGGGCTGTGGGAGAATCAATGTGTTTTGTTTCTAAGCCGCCCAGCCTCTGGTAT
TCTGTGACAGCAGCCTGAGATGGACTAAGACATCTCATAAGAAGAGGAGATGAGGACACAGACACACACCAAGGG
ACAACCCTGTGGGACACGGAGAAGACGGGGTCTCCAAGCCCAGGAGAGAGGCCTCAGGAGGAACCAGCCCTGCCC
ATGCCCATCTCTTGATCTTGGACCTGCAGCTTCCAGGACTGTGGGAGAATCAATTCCTTTTTTCTTTTTCTTTTT
TCTTTTTTTTCCTCTTTTTCTGAGATGGAGTCTCACTCTGTCGCCAGGCTGGAGTGCAGTGGCGTGATCTTGGCT
CACTGCAACCTCCACCTCGCAGGTTCAGGCCATTCTCCTGCCTCAGCCTTCTGAGTAGCTGGGATGACAGGCGCC
CGCCACCACACCCGGCTACTGTTTGTATTTTTATTAGAGACGGGCTTTCTGGCCATGTTGGCCAGGCTGGTCTCA
TACTCCCGACCTCAGGTGATCCGCCCGCCTTGGCCTCCCAAAGCGCTGGGATTACAGGCGTGAGCCACCGCGCCC
GGCCAAATTCCTTTTTTCTGAAGCCACCAAGCTGTGGGACTTTTTTATGGCAGTCCCAGCAGACAGATCCACCCT
CGTTCCAAATAAGGTTATCGTCATAGGTTCTGGGGGTGAGGTCATAGATGTATGTTTTTTTTTTTTTTTGGAGAG
AGAGTCTCTCTCTATTGCCCAGGCTGGAGGGCAGTGGCGCGATCACGGCTCACTGTAGCCTCGACCTCCTGGGCT
CAAGCGATCCTCTCGGGAGGCTAAGAGAGGAGAATCGTTTGAATCCGGGAGGCAGAGGTTGCAGTGAGCTGAGAT
AGCACCACAGCACTGCAGAGCGAGACTCTGTCTCAAAAAAAAAAAAAAGAAAAAAGAAAAAAAATAGGCCGGGTG
CGTGGCTGACGCCTGTAATCCCAGCACTTTGGGAGGCCGAGGCGGGTGGATCACTTGAGGTCAGGAGTTCAAGAC
CAGCCTAACGAAGATGGTGAAACCCCATCTCTATGAGAAATACAAAAATTACCCGGGCGTGGTGGCGGGCGCCTG
TAGTCCCAGCTACTCGGGAGGCAGGGGCAGGAGAATCGCTTGAACCTGGGAGGTGGAGGTTGTGAGCTGAGATCG
TGCCACTGCACTCCAGCCTGGGCGACAAAACGAGACTCCATCTCAAAAAAAAAAAAAAAAGAAAAAGAAAAAGAA
AAAATTAACACACACACACAAATTCCATGATAAACACAAAATCAAGTTCAAAGCACGCACGCCAGTGCTGCCTCT
GCTGCCCCTCCATGCTGCCCACACCCAAAGCACCTGCCGGCCTGCTGGGCACAGTGGAAGTCCCTGGCGCTCCCC
GTGTCCCGAGCACCAGGCAGACAGGGATCCCTGGTGGTCTTTCCGCTCCCTTAGGGTTCCAGACTGGGGCTGGGA
GGTCCGGAAGTCGCTCCCGGTCCTGGTACTCAGGTGGCCTGCAGGTGGCCCGCAGGTGGTCACGGTCTCTGTGCA
GGTGGCACTACTGGGGTGTCCCCCCCTGGACGCCACCCCATATGGCAGCCACCTCTCTGCTTCCCAGGGCCCCGG
GGAGAGCTTACAGTCCCTGGTCCCCCCAGGACGGCCCCCGGTCTGTGACCCTCTCACCCTTTACCCCTAGAGTGC
GACCAGGAGGAGGGCGCAAACACACGTGCCTGGCGGACGTCGCTGCTGATCGCGCTGGGGACGCTGCTGGCCCTG
GTCTGTGTCTTCGTGATCTGCAGAAGGTGAGCCCTCGAGGGCGTCCGCGAGCGTCGCTTGTTTCCAGTGTGACCC
TGAAAGTTATTCACAGAACCATCCTGAGAATTATCATTATTATTTTTGTGATGGAGTCTCGCTCTGTCTCCCAGG
CTGGAGTGCAGTGGCACGATCTGGGCTCACTGCAGCCTCTGCCTCCTGGGTTCAGGCGATCCTCCTGCCTCAGCC
TCCCAAGTAGCTGGGATTACAGGCACCCAGCAACACACCCAGCTAATTTTTGTATTTTTAGTAGAGACGGGGTTT
TGCCATGTTGGCCAGGCTGGTCTTGAATTCCTGGACTCAGGTGATCCACCTGCCTCAGCCTCCTAAAGTGCTGGG
ATTACAGGCGTAAGCCACCTCGCCTGGCCCATATTATTATTATTGTTATGATTATTATTATTTTTTGAGTCTTGC
TCTGTCACCCAGGCTGGAGTGCAGTGGCTCGATCTCGGCTCACCGCAGCCTCCACCTCCCGGGTTCAAGTGATTC
TCCTGCCTCAGCCTCCCGAGTAGCTGGGATTACAGGTGTGCAACACTGCACCCAACTAATTCTTGTATTTTTAGT
GGAGACGGGGTTTCACCATGTTGGCCACACTGGTGTTGAACTCCTGGCCTCAAGTGATCCCCCAGCCTCGGCCTC
CCAAAGTGCTGGGATTACAGGCGTGAGCCACCGCACCTGGCCCCTTGAGAATTATTCACAAAAGACCAGGGGCCA
GGTCCTCTCTGCTAGCTGCCCCCTGCCAGGAGGTCTGTAGAGCCAAAGCTGAGGGCTCTGGTGGGACCAAGGGTG
TCCCCAGGGTGGAGGAAGCAGGGCCGGTCCTCCCCTGCCCTGCCCAGCGGGCCTGACACAGTCAGAGGGCGAAAG
GCCAGGCTTTCTGGTCGGGAAGGGGCCTGGGCGAACGTCACAGCTGTCCACTTGGATGGGCCAGGATCCGTCATG
CAGACCAACTCGAGGTTTTTGGTCACCAGCTGGGTCACCCCAGGTCCTGTCTCCTTTTTTTTAGAGAAGGAGTTT
TGCTTTTGTTGCCCAGCCTGGAGTGCAATGGCGCCATCTCGGTTCACTGCAACCTCTGCCTCCTGGGTTCAAGCG
ATTCTCCTGCCTCAGCCTCCCTGAGTAGCTGGGATTACAAGGATGTGCCACCACGCCTGGCTAATTTTGTATTTT
TAGTAGAGATGGGGTTTCTCCGTGTTGGTCAGGCTGGTCTTGAACTCCTGACCTCATGTGATCCACCCGCCTCGG
CCTCCCAAAGTGCTGGGATGACAGGCGTGAGCCACCATGCCCGGATAATTTTGTATTTTCAGTAGAGACGGAGTT
TCTCCGTGTTGATCAGGCTGGTCTTGAACTCCTCACCTCAGTTGATCTGCCCGCCTGGGCCTCCCAAAGTGCTGG
GATGACAGGCGTGTGCCACCATGCCCAGATAATTTTGTATTTTTAGTAGAGATGGAGTTTCTCCATGTTGGTCAG
GCTGTTCTCGAACTCCTGACCTCAGTTGATCTGCCCGCCTGGGCCTCCCAAAGTGCTGGCATTACAGGCTTGAGC
CACCATGACCAGCCAGGCCCTGTTTCCTTCCTGCTGGGCATCAGAGCTTCACAATCTGTCAAATGGGTGCCATGG
TGACTTCTGTCTCCCGGGGGAAGGGGGAAAAGGGGAGGGGGAGGGGGAAGGGGAAGAGGGGGGAGGAGGGGGGAA
GGGAGGAGGGGGAAGGGAGGGGGGAAAGGAGGGGGAAGGGAGGAGGGGGAAGGGAAGAGGGGGAGGAGGGGGAAG
GGGAGGAGGAGGAGGGGGAGGAGAGGCGAGGGGGAGGGTGGGGGAGGGGGAAGGGGGAGGAGGAGAGGGGAGGAT
GAGTGGGGGGGGGAAGGAGGGAGAGGGGGAGGGGGAGAGGGAGGGGTAGGGGGGAAGGGGTGGAGGAGGGAGGA
AAAGGGGGAGGAAGAGGGGGACGAGGCCCTTCCTGCAAGCTGTCTCAGGTCGTAAACTCAGTGACCTGAGGCACC
AGGGGTCTGTCTTTGCAGCTGCACCCCTGGGTGGCTCCGGTACCAGCGCCCTACTCCTTTAATTAGACACCAGCG
CCTGCCTATGATGATGGTCGGGGGCGTGTCAGGGCCTCAGGGGCCGGGAAAATAGAGACCCCTCGAGTAGATGAC
TTGAGTCTTTTGCTCTGTCCTGGCACTGTCTGTCCTGGACACGCTGTGTCCCAGATGATGAGCTGGTCGGTTTTG
GGTTCAGAGCTGGGCCATTTCTCTTTCCTCCGAGGTATCTGGTGATGCAGAGACTCTTTCCCCGCATCCCTCACA
TGAAAGACCCCATCGGTGACAGCTTCCAAAACGACAAGCTGGTATGTTGTTTTTTCTGCCTTGGGACGGGTCTGG
AGGCGTGGTGGCCACTTTGGGAGGCCCAGGCGGGCGGACCACTTGAGGCCAGGAACTGGAGACCAGCCTGGCCAA
CATGGAGAAACTCCGTGTCTACTAAAAATACAAAATTAGCCGGGTGTGGTGGCGGGCGCCTGTAATCCCAGCTAC
TCGGGAGGCTGAGGGAGGAGGATCACTTGAACCCGGGAGGTGGAGGCTGCAGTGAACTACGATCGAGCCACTGCA
CTCCATCTGGGCGACAAGAGTGAAATGGCCTGGACTAGGAGGCTGGACACCTGTCTGCTGGCGCTGGTGGGCGGC
TGGGAAAAGCTGCAGGATGAAGGGAGTGGGGGGACACTGGGCTCCCAGCCCACCGTGATCACGGGCCGTCTCCGC
ACCCTGCACACCAGAGCAGAGCAGATTTTTTTTTTTTTTTTTAGATGGAGTTTTGCTCTTGTTGCCCAGGCTAGA
GTGCAATGGTGCGATCTTGGCTCACCACAACCTCCACCTTCCCAGTCCAAGGTTCAAGCGATTGTCCTGCCTCAG
CCTCCCGAGTAGCTGGGACTACAGGCACGCACCGACACGCCCAGCTAATTTTGTATTTTTAGTAGAGACGGGGTT
TCACCATGTTGGCCAGGATGGTCTCGATCTCCTGACCTTGTGATCCCCCTGCCTCGGCCTCCCAAAGTGCTGGGA
TTACAGGCATGAGCCACCATGCCCAGCCTAGTTTTCAACAAGTTTTTAGTGATACCTGTGTCCCTAAGAGAAAGG
AAGGGCAGAGGAAAAGGAGGCAGACATCTCTGTCAGAGTTTTTTGTTTTGTTTTGTTTTGTTTGTTTTTGTTTTT
GAGACGCAGTTTTGTTCGTTGCCCCGGCTGGAGTGCAATGGTGCAATCTCGGCTCACTGCAACCTCCACCTTCCC
GGTTGAAGGTTCAAGCGATTCTCCTGCCTCAGCCTCCCAAGTAGCTGGGACTACAGGCATGCACCAACACACCCA
GCTAATTCTGCATTTTTAGTAGAGAAGGGGTTTCACCATGTTGGCCAGGATGGTCTGGATCTCCTGACCTTATGG
TCCGCTCGCCTCGGCCTCCCAAAGTGCTGGGATTACAGGTGTGACCCACCGCGCCTGGCCCAAAGTGCTGGGATG
ACAGGCGTGAGACACCATGCCTGGCCCACAGAGCAGATCTGAGATGGGACAGGCCCCCGCAGATCAGGACGTGGG
CTCTGTTATCTGGGGGGTGGCCGACTCACCCTGCCTCCTCTCGTCTCTGCAGGTGGTCTGGGAGGCGGGCAAAGC
CGGCCTGGAGGAGTGTCTGGTGACTGAAGTACAGGTCGTGCAGAAAACTTGAGACTGGGGTTCAGGGCTTGTGGG
GGTCTGCCTCAATCTCCCTGGCCGGGCCAGGCGCCTGCACAGACTGGCTGCTGGACCTGCGCACGCAGCCCAGGA
ATGGACATTCCTAACGGGTGGTGGGCATGGGAGATGCCTGTGTAATTTCGTCCGAAGCTGCCAGGAAGAAGAACA
GAACTTTGTGTGTTTATTTCATGATAAAGTGATTTTTTTTTTTTTAACCCA

A representative mRNA sequence of CD123 is provided by NCBI Reference Sequence No: NM_002183.4, shown below:

```
  (SEQ ID NO: 14)
      1 cttcggtttc tcttcgggga aagctgcttt cagcgcacac gggaagatat cagaaacatc
    61 ctaggatcag gacaccccag atcttctcaa ctggaaccac gaaggctgtt tcttccacac
  121 agtactttga tctccattta agcaggcacc tctgtcctgc gttccggagc tgcgttcccg
  181 atggtcctcc tttggctcac gctgctcctg atcgccctgc cctgtctcct gcaaacgaag
  241 gaagatccaa acccaccaat cacgaaccta aggatgaaag caaaggctca gcagttgacc
  301 tgggacctta acagaaatgt gaccgatatc gagtgtgtta aagacgccga ctattctatg
  361 ccggcagtga acaatagcta ttgccagttt ggagcaattt ccttatgtga agtgaccaac
  421 tacaccgtcc gagtggccaa cccaccattc tccacgtgga tcctcttccc tgagaacagt
  481 gggaagcctt gggcaggtgc ggagaatctg acctgctgga ttcatgacgt ggatttcttg
  541 agctgcagct gggcggtagg cccgggggcc cccgcggacg tccagtacga cctgtacttg
  601 aacgttgcca acaggcgtca acagtacgag tgtcttcact acaaaacgga tgctcaggga
  661 acacgtatcg ggtgtcgttt cgatgacatc tctcgactct ccagcggttc tcaaagttcc
  721 cacatcctgg tgcggggcag gagcgcagcc ttcggtatcc cctgcacaga taagtttgtc
  781 gtcttttcac agattgagat attaactcca cccaacatga ctgcaaagtg taataagaca
  841 cattccttta tgcactggaa aatgagaagt catttcaatc gcaaatttcg ctatgagctt
  901 cagatacaaa agagaatgca gcctgtaatc acagaacagg tcagagacag aacctccttc
  961 cagctactca atcctggaac gtacacagta caaataagag cccgggaaag agtgtatgaa
1021 ttcttgagcg cctggagcac cccccagcgc ttcgagtgcg accaggagga gggcgcaaac
1081 acacgtgcct ggcggacgtc gctgctgatc gcgctgggga cgctgctggc cctggtctgt
1141 gtcttcgtga tctgcagaag gtatctggtg atgcagagac tctttccccg catccctcac
1201 atgaaagacc ccatcggtga cagcttccaa aacgacaagc tggtggtctg ggaggcgggc
1261 aaagccggcc tggaggagtg tctggtgact gaagtacagg tcgtgcagaa aacttgagac
1321 tggggttcag ggcttgtggg ggtctgcctc aatctccctg gccgggccag gcgcctgcac
1381 agactggctg ctggacctgc gcacgcagcc caggaatgga cattcctaac gggtggtggg
1441 catgggagat gcctgtgtaa tttcgtccga agctgccagg aagaagaaca gaactttgtg
1501 tgtttatttc atgataaagt gatttttttt tttttaaccc a
```

A representative amino acid sequence of CD123 is provided by NCBI Reference Sequence No. NP_002174.1, shown below:

(SEQ ID NO: 15)
MVLLWLTLLLIALPCLLQTKEDPNPPITNLRMKAKAQQLTWDLNRNVTDIECVKDADYSMPAVNNSYCQF
GAISLCEVTNYTVRVANPPESTWILFPENSGKPWAGAENLTCWIHDVDFLSCSWAVGPGAPADVQYDLYL
NVANRRQQYECLHYKTDAQGTRIGCREDDISRLSSGSQSSHILVRGRSAAFGIPCTDKFVVFSQIEILTP
PNMTAKCNKTHSFMHWKMRSHFNRKFRYELQIQKRMQPVITEQVRDRTSFQLLNPGTYTVQIRARERVYE
FLSAWSTPQRFECDQEEGANTRAWRTSLLIALGTLLALVCVFVICRRYLVMQRLFPRIPHMKDPIGDSFQ
NDKLVVWEAGKAGLEECLVTEVQVVOKT

The present disclosure provides a number of CD38 target sites and corresponding gRNAs that are useful for targeting an RNA-guided nuclease to human CD38. Table 3 below illustrates preferred target domains in the human endogenous CD38 gene that can be bound by gRNAs described herein. The exemplary target sequences of human CD38 shown in Table 3, in some embodiments, are for use with a Cas9 nuclease, e.g., SpCas9.

TABLE 4
Exemplary Cas9 target site sequences of human CD38 are provided, as are
exemplary gRNA targeting domain sequences useful for targeting such sites.
For each target site, the first sequence represents the DNA target
domain sequence, the second sequence represents the reverse complement
thereof, and the third sequence represents an exemplary targeting domain
sequence of a gRNA that can be used to target the respective target site.

| gRNA Name | gRNA Alternate Name | Target domain sequence |
|---|---|---|
| CD38_CBE_g112 | CD38_CBE S_g27 | TGAGTTCCCAACTTCATTAG (SEQ ID NO: 16) |
| | | CTAATGAAGTTGGGAACTCA (SEQ ID NO: 17) |
| | | UGAGUUCCCAACUUCAUUAG (SEQ ID NO: 18) |
| CD38_CBE_g117 | CD38_CBE_S_g28 | TCAGACCGTACCTTGCAACA (SEQ ID NO: 19) |
| | | TGTTGCAAGGTACGGTCTGA (SEQ ID NO: 20) |
| | | UCAGACCGUACCUUGCAACA (SEQ ID NO: 21) |
| CD38_CBE_g119 | CD38_CBE S g29 | GTACCTTGCAACAAGGTAAT (SEQ ID NO: 22) |
| | | ATTACCTTGTTGCAAGGTAC (SEQ ID NO: 23) |
| | | GUACCUUGCAACAAGGUAAU (SEQ ID NO: 24) |
| CD38_CBE_g120 | CD38_CBE_S_g30 | TACCTTGCAACAAGGTAATT (SEQ ID NO: 25) |
| | | AATTACCTTGTTGCAAGGTA (SEQ ID NO: 26) |
| | | UACCUUGCAACAAGGUAAUU (SEQ ID NO: 27) |
| CD38_CBE_g121 | CD38 CBE S_g31 | ACCTTGCAACAAGGTAATTG (SEQ ID NO: 28) |
| | | CAATTACCTTGTTGCAAGGT (SEQ ID NO: 29) |
| | | ACCUUGCAACAAGGUAAUUG (SEQ ID NO: 30) |
| CD38_CBE_g122 | CD38_CBE_S_g32 | CCTTGCAACAAGGTAATTGG (SEQ ID NO: 31) |
| | | CCAATTACCTTGTTGCAAGG (SEQ ID NO: 32) |
| | | CCUUGCAACAAGGUAAUUGG (SEQ ID NO: 33) |
| guide-120 | N/A | AATCGATTATAAGCAAAAGG (SEQ ID NO: 34) |
| | | CCTTTTGCTTATAATCGATT (SEQ ID NO: 35) |
| | | AAUCGAUUAUAAGCAAAAGG (SEQ ID NO: 36) |
| guide-121 | N/A | AAAATTGAATATTCCTTTTG (SEQ ID NO: 37) |
| | | CAAAAGGAATATTCAATTTT (SEQ ID NO: 38) |
| | | AAAAUUGAAUAUUCCUUUUG (SEQ ID NO: 39) |
| guide-122 | N/A | AATATTCAATTTTCCTGCAA (SEQ ID NO: 40) |
| | | TTGCAGGAAAATTGAATATT (SEQ ID NO: 41) |
| | | AAUAUUCAAUUUUCCUGCAA (SEQ ID NO: 42) |
| CD38_S_BE_g83 | guide-266 | AATCGATTCCAGCTCTTTTA (SEQ ID NO: 43) |
| | | TAAAAGAGCTGGAATCGATT (SEQ ID NO: 44) |
| | | AAUCGAUUCCAGCUCUUUUA (SEQ ID NO: 45) |
| CD38_S_BE_g75 | N/A | GATTCCAGCTCTTTTATGGT (SEQ ID NO: 46) |
| | | ACCATAAAAGAGCTGGAATC (SEQ ID NO: 47) |
| | | GAUUCCAGCUCUUUUAUGGU (SEQ ID NO: 48) |
| CD38_S_BE_g76 | N/A | CGATTCCAGCTCTTTTATGG (SEQ ID NO: 49) |
| | | CCATAAAAGAGCTGGAATCG (SEQ ID NO: 50) |
| | | CGAUUCCAGCUCUUUUAUGG (SEQ ID NO: 51) |
| CD38_S_BE_g77 | guide-270 | TGGAATCGATTATAAGCAAA (SEQ ID NO: 52) |
| | | TTTGCTTATAATCGATTCCA (SEQ ID NO: 53) |
| | | UGGAAUCGAUUAUAAGCAAA (SEQ ID NO: 54) |
| CD38_S_BE_g78 | guide-273 | TTTCCTGCAAGAATATCTAC (SEQ ID NO: 55) |
| | | GTAGATATTCTTGCAGGAAA (SEQ ID NO: 56) |
| | | UUUCCUGCAAGAAUAUCUAC (SEQ ID NO: 57) |
| CD38_S_BE_g79 | guide-274 | TTACCTGTAGATATTCTTGC (SEQ ID NO: 58) |
| | | GCAAGAATATCTACAGGTAA (SEQ ID NO: 59) |

The present disclosure provides exemplary CD38 targeting gRNAs that are useful for targeting an RNA-guided nuclease to human CD38. Table 5 below illustrates preferred targeting domains for use in gRNAs targeting Cas9 nucleases to human endogenous CD38 gene. The exemplary target sequences of human CD38 shown in Table 5, in some embodiments, are for use with a Cas9 nuclease, e.g., SpCas9.

TABLE 5
Exemplary Cas9 targeting domain sequences of gRNAs targeted to human
CD38 are provided.

| gRNA Name | gRNA Alternate Name | Targeting domain sequence |
|---|---|---|
| CD38_CBE g112 | CD38_CBE_S_g27 | UGAGUUCCCAACUUCAUUAG (SEQ ID NO: 18) |
| CD38_CBE_g117 | CD38_CBE_S_g28 | UCAGACCGUACCUUGCAACA (SEQ ID NO: 21) |
| CD38_CBE_g119 | CD38_CBE_S_g29 | GUACCUUGCAACAAGGUAAU (SEQ ID NO: 24) |
| CD38_CBE_g120 | CD38_CBE_S_g30 | UACCUUGCAACAAGGUAAUU (SEQ ID NO: 27) |
| CD38_CBE_g121 | CD38_CBE_S_g31 | ACCUUGCAACAAGGUAAUUG (SEQ ID NO: 30) |
| CD38_CBE_g122 | CD38_CBE_S_g32 | CCUUGCAACAAGGUAAUUGG (SEQ ID NO: 33) |
| guide-120 | N/A | AAUCGAUUAUAAGCAAAAGG (SEQ ID NO: 36) |
| guide-121 | N/A | AAAAUUGAAUAUUCCUUUUG (SEQ ID NO: 39) |
| guide-122 | N/A | AAUAUUCAAUUUUCCUGCAA (SEQ ID NO: 42) |
| CD38_S_BE_g83 | guide-266 | AAUCGAUUCCAGCUCUUUUA (SEQ ID NO: 45) |
| CD38_S_BE_g75 | N/A | GAUUCCAGCUCUUUUAUGGU (SEQ ID NO: 48) |
| CD38_S_BE_g76 | N/A | CGAUUCCAGCUCUUUUAUGG (SEQ ID NO: 51) |
| CD38_S_BE_g77 | guide-270 | UGGAAUCGAUUAUAAGCAAA (SEQ ID NO: 54) |
| CD38_S_BE_g78 | guide-273 | UUUCCUGCAAGAAUAUCUAC (SEQ ID NO: 57) |
| CD38_S_BE_g79 | guide-274 | UUACCUGUAGAUAUUCUUGC (SEQ ID NO: 60) |

TABLE 6
Exemplary targeting domain sequences of gRNAs targeted to human CD38 using base editors
(e.g., ABE or CBE) comprising SpRY Cas9 or SpG Cas9 are provided.

| gRNA | Targeting domain | PAM | BE | | | |
|---|---|---|---|---|---|---|
| Name | sequence | Sequence | Sequence | Codon | Amino Acids | Consequence |
| 9991 | AATATTCAATTTTCCTGCAA | GAA | TgTTCa | ATT/GTT | I->V IQ->VR | Missense |
| | (SEQ ID NO: 40) | | TgTTCg | ATTCAA/GTTCG | | variant |
| | | | | A | | |
| 9992 | ATATTCAATTTTCCTGCAAG | AAT | gTTCaa | ATT/GTT ATTCAA/GTTCG | IQ->VR | Missense |
| | (SEQ ID NO: 105) | | gTTCga | A ATTCAA/GTTCG | I->V IQ->VR | variant |
| | | | gTTCgg | G | | |
| 9995 | ATTCAATTTTCCTGCAAGAA | TAT | TCgaTT | CAA/CGA | Q->R | Missense |
| | (SEQ ID NO: 106) | | TCggTT | CAA/CGG | | variant |
| 9684 | TTCCCGCAGGGTAAGTACCA | AGT | CCCGCg | AGG/ GGG | R->G | Splice region |
| | (SEQ ID NO: 107) | | | | | variant |
| 9686 | CCCGCAGGGTAAGTACCAAG | TAG | CGCgGG | AGG/GGG | R->G | Splice region |
| | (SEQ ID NO: 108) | | | | | variant |
| 9689 | CCGCAGGGTAAGTACCAAGT | AGT | GCgGGG | AGG/GGG | R->G | Splice region |
| | (SEQ ID NO: 109) | | | | | variant |
| 9690 | GCAGGGTAAGTACCAAGTAG | TGA | gGGGTa | AGG/GGG | R->G | Splice region |
| | (SEQ ID NO: 110) | | gGGGTg | | | variant |
| 9388 | TGGGAACTCAGACCGTACCT | TGC | GGgaCT | GGA/GGG | G GT->GA | Missense |
| | (SEQ ID NO: 111) | | GGggCT | GGAACT/GGGGC | | variant |
| | | | | T | | |
| 9389 | GGAACTCAGACCGTACCTTG | CAA | agCTCa | ACT/GCT GGAACT/GGGGC | GTQ->GAR | Missense |
| | (SEQ ID NO: 112) | | ggCTCa | T GGAACTCAG/GG | T->A GT->GA | variant |
| | | | ggCTCg | GGCTCGG | | |
| 9390 | GAACTCAGACCGTACCTTGC | AAC | gCTCaG | ACT/GCT | T->A TQ->AR | Missense |
| | (SEQ ID NO: 113) | | gCTCgG | ACTCAG/GCTCG | | variant |
| | | | | G | | |
| 9391 | ACTCAGACCGTACCTTGCAA | CAA | TCgGaC | CAG/CGG | Q->R QT->RA | Missense |
| | (SEQ ID NO: 114) | | TCgGgC | CAGACC/CGGGC | | variant |
| | | | | C | | |
| 9393 | CTCAGACCGTACCTTGCAAC | AAG | CgGaCC | CAG/CGG | Q->R QT->RA | Missense |
| | (SEQ ID NO: 115) | | CgGgCC | CAGACC/CGGGC | | variant |
| | | | | C | | |
| 9396 | TCAGACCGTACCTTGCAACA | AGG | gGaCCG | CAG/CGG | Q->R QT->RA | Missense |
| | (SEQ ID NO: 19) | | gGgCCG | CAGACC/CGGGC | | variant |
| | | | | C | | |
| 9397 | CAGACCGTACCTTGCAACAA | GGT | GgCCGT | ACC/GCC | Q->R QT->RA | Missense |
| | (SEQ ID NO: 116) | | | | | variant |

The gRNAs of Table 6 are designed for PAM flexible (SpG Cas9, PAM = NG) or PAMless (SpRY Cas9, PAM = NRN) Cytosine and adenine base editors (CBEs and ABEs).

A representative DNA sequence of the CD38 gene is provided by NCBI Gene ID: 952, shown below.

(SEQ ID NO: 61)
GWCAGTTTCAGAACCCAGCCAGCCTCTCTCTTGCTGCCTAGCCTCCTGCCGGCCTCATCTTCGCCCAGCCAACCC
CGCCTGGAGCCCTATGGCCAACTGCGAGTTCAGCCCGGTGTCCGGGGACAAACCCTGCTGCCGGCTCTCTAGGAG
AGCCCAACTCTGTCTTGGCGTCAGTATCCTGGTCCTGATCCTCGTCGTGGTGCTCGCGGTGGTCGTCCCGAGGTG
GCGCCAGCAGTGGAGCGGTCCGGGCACCACCAAGCGCTTTCCCGAGACCGTCCTGGCGCGATGCGTCAAGTACAC
TGAAATTCATCCTGAGATGAGGTGGGTTGGCGACTAAGGCGCACCGGTGGGCACTGCGGGGACAGCAGGGCCCCG
CGCGCAGGGAAGCCGCCCGGATCGCCCGGAACCGGGCATCTTCCGTGGCGGGTCAGCCGAGAGCCCGCCGGGTGG
TGCTGAGTAGGGAGTCCCGGGCTCGGGGCTCCGCGGGCCGCTTTCAGGAGCAGCTGGCCTTGGCACCGAGCGTGC
CCGCGGGAGGCGGGGGGGGGCGCTGCTCGGTGGCTCTGCTGCGTAGCCGGTGAACACTTGGCACCGATGCCCGCC
TTCTGGGCAAGGTGCCCTGAGCCCAGCCCCTCGCCGGGCTGCAGCCCACCCTCGGCGCGCTCAGCCCGCTTCACC
GCTTCAGGGACGGAATAGAACTCGCAGATGCAGGGTGTCGCTGACATTTTCAACTTTTTCTGCGGTTTCCGCCCG
CTGTCTCTGACCCGAAAGTGCCCCCGGACGGTTACAGAGGACACTTAAGTGGTTTGCAAAGCCTGTGGTAGGGGA
GGAGGGTGTAGAAGGGCCAAACCACGGAACTTAGTTTTATTCATTTATATAAAGCAGCACTCCGATTCTTTTTGC
GCGGCCTGAAATGCATGTGACCAGAGAAGTAATTAACAAAACAATGTCAACTTCTAAAACCGAGACATTACTTAG
ATGATAAGGCGCAGCAACTCGGTGAATCTGTACAAACCTTGGAAAAAAAACACATTAGTCTATGGGACCTTCCAG
TTTTCTCATGCTCCTTTCCAGCTACTAACCTCTCCTAAAGGGAACAACCACTTTTTGGATTTGATTCCCAGGCCT
CGCTTTCACCGGGAAATTATCGTTGCTTGTAAAACAGAAGAAGCCGGGAAGGCAGGCAGGGGGAGCTGCTACTTT
ACACTCTGTGCTTTGGGATAGCAAAATCCCGCATTTAAGCAATCCGAGGAAACGAGCAAATAGACCTCCCTCGCC
TCTCCGAGCACACTCAACAGTTCCGGTTGCAAAATGTTTGCCTCCTGGGCTTCCCAGCGTCCCGTTAGTTGTTCT
ATTTACACATAATTAGATACTTAATGGAGAGAGAAACTAGAAGTTGAGGCGTTCCTCCAGGCTGTATTGTAAAGT
ATGAAGTGAAATCCAAAATGAAATGGTAATGTTAGAAAGCAACCTCATTAAAAAAAAAAAAAGTAACACTGGTCT
TGAAGATCTTTCAATGTGAGTACATAAAGATCTATCTCATTTCTTTTGACAGCCCATAGTATTTCATAAACTAGA
TGTAACCATTTCCTATTGACAGGAAATTAGCTTGTTTCCAATTTTTCAATCCCATTCATTCATCCAACAAGTATC
TGTTGAGCACCCACTATGTTCCAGACAGTGATCTAGCTACTGATGACACAAGAGTGAATGACGAAGTTCTCACTC
ATGAAATTTTCACCTTAGTTGGGAGAAACATGATGCAATGAAAATCTTCATACATACATTGTGTGTACATATGGG
AGTATTTCTGTAGGATAGATTTCTAGTGATGAAACTGCTGAGTAAAAGGGAGAATTATGCATATTTTAAGTTTTG
ATTTTTCCAAATTCCAGGTATTCCATATATACTCCAAAATAGTTGTGCCATTTTACTCTCCCATCATCAGTCTAT
CAGAGGGGATGCTTTCCCACAATCTCTTGAATGCTGAATATTTTCAACTTTTTTACTTAAGAGAAAAAAGAGCAT
CTAATTGTTCCCTCAGTATCAGTGAGTCTAAGCATCTTGTATATGTTTATTTGCCATTTATATTTTTTTCTGTGA
TTTTCCTGTCCAGATACTTGATACTTTCTATTGAGCTGCTTATTTATTTCTTATGGGAGATTTTTATATATTTTA
GATAATATTCTCTCTCTCACACACACACACACACACACACACACACACACACACACACACACACACATACAGTCT
TACAGCCACATCCCTGAAATCTTGACCTTGTGAACATGTTTTACTGGCAGCACTCTGGACTCGATCATTGCCTTG
AGACTATTTCTTTTTTGATATTCTTTGGAAAGACTAACAATGACAGTTTTATTTTCAAACCCAACAAATCCTGGC
ATGGAAATGTTTGCTCTTGATTCTGCTTTTAAAAAAATAAAGAATTATTTTCTCTCTTTCTTTCTGCACCTTATC
AGAAACAGCTAAAAGAAGTGAGTTGGGCCAGGCACTGTGGCTCACACCTGTAATCCCAGCACTTTGGGAGGCCCA
GGCAGGTGGATCACTTAAGGTCAGGAGTACAAGACCTGCCTGGCCAACATGCTGAAACTCCGTCTCTACTAAAAA
TACAAAATTAGCCGGGTGTTGTGGCGCGTGCCTGTAATCCCAGCTACTCTGGAGACTGAGGTGGGAGAATCGCTT
GAACCCAGGAGGAGGAGGTAGCACTGAACCAAGATCCAGCCTGGCCAAGAGAGTAAGACTCCGTCTCAAAACCAA
ACCAAACCAAACCAAAAAAAGAAGTGAGTTGGCACTTTCAACATTCTGCCTGGAAATCTCCTTACCAAACCTATA
AGATCATTAGGTATATTTTCTGCACTTTGTATTGTGACAGGTGACAGTGTTACCAAACTTTTTACCAGGACATAA
TAGGGTCTGCCTTTCTTCTAGTTGCTAACAATTTCCCCCAAGTCCATCTTGCCTGCACTAACAGTCTCCTTTAGA
CCTCTCCTCTCCTGCCTGTCACACATTCCTAGTACTAATGCTACAGTATAGTAGTAAGGGTCTCCAGAGAAACAA
CATTTATATAACATAATATAAATACATTAATAGAGAGAAAGAGATTTTAAGAAATTGGCTTATATCATTGTGAAG
TCGGGCAAGCCCCAAATCTGCTGGACAGGCCAGCAGCCTGGAGACCCAGGGAAGAGTTGATGTTGCAGCTGGAGT
CCAAAGGCAGTCTCTGGCAGAATTCTCTTTTACTTCTGGGACCTTGGTCTTTCTCTTAAGGCCTTCAACTGATTG
GATGAGGCCCACCACATTATGAAGGGTAACATGCTTTACACCGAGTCTCCTGACTTAAAATCTAAAAAATACCTT
CACATCACAACTAGATGTGTTTGACCAAATATCTGGATACCATGCCTGGGCGAATTGCCACTTAAAATTAATCAT
CACGTACATGTTTTAAGGTTTTGTTACACAAGACCTCACTTCCAAGTATCACTTTCTGTTTTGGTCATCAGTTGC
TGCATAATAAACAACCACCCTAAAATTTAGTGACTTAAAACAACAATCATTTATTGTCTGCCATGGTTCTGTGGT
TTGACTGGGATCAGCTGAGTGGCCTGTTTCACTTGGTGTCAGCTGGGGGTGTAGGCATCTGCAACATTGTCTTGG
CAGGAACATCCAAGATGTCCCACTTAACACGATGGCTCCTGGGCTCAGCTGGGCTGGTCAGGCCTCCCTTCCTCT
CTGTGTTGCCACACGGCCTCTCTCTATCCATGTGGCCTCTCCATATGGTCTCTCCCTGGTGGAGGTGAATTTCTT
CAAGGTTTCTAAACTCTCAAAAGTGGAGCCTGGCAAGCCCACTCAAAGCTTCAGATCCACAACTGGCACAGCTTC
CCTTCCACAGATTCTATAGGTTAAAACAATCACCGGACCAGCCCAGATTCAAAAGAAGGAGAAACAAACTCCACC
CCTCCATGGAGGAAGTAGCAAAAATAATGTAGACAGTTTTTACCCTTTACATCTGGATTTGTTAGCTTTTCTGTT
TTCATTTTCTTCTGGTCTTTCTTTTGTCCTTCTCTTACACTTATTTTATTACCTTTTATCAATTAGCTTTTAAGA
TGATAAAAATCTAATACATGCCTCCTGGAATGTCTTCTTGAGCCTAGGGACTTTTGTTTAATGATATATCTTGAG
GACCTAGAATAGTGCCTGAAATACAATAGTCATTAAATATTTAGCTGAATTAAATGAATGGTATATAAGCCAGGG
TATTAAAAATAACATAAACAAAGTTGTAATAAATATACTTCCCCAGTGAACGACCTAATACCATTACTCCCCAAA
CCCTCAATTTCTGTCTTGAGCATAGAAACTGTTAATTTTTCCTTTGTGTAGTAGGTCCTTAGTATTTCTTTAGAG
GTTGTAGCACTTTATCTTCCTCACTGTTCCTTTTCCTTGGTTGTCCTTTTCCAAACATCTCTCAACAATTTCTCC
CAAGTCCATCTTGCCTGCACTAACAGTCTCCTTTAGAACTCCCCTCTCCTGCCTGTCACACAGTCTTAGTAATAG
TGCCACAGTATAGTAGTAAGGGTCTCCAGAGAAACTCACTTTCTGCAAGTTTTTTAGTGTGGGTAGGTGAGTATT
GAAGCTTGTTCTTGGCGTTACCAGGTTGGTTCTTTGAGTTGAACCAGGGGCATTACATGCGGAATATTCCTGAAC
AGATCACCTCTGGTTCTGCTGTCTCAAGGGCCACACACAAGAGCTGCCTTCTGACCAAGATGTCTCTGGGCACAT
GAGACCTGAAATACACATGGCCAAGACTCAACAAAGCGTTTGCTGACTGTCAGAGCTGACAGCATCTCGGTACTG
TGGGAGGGAGCCCAGTGTCTGGTGATAGTCAGGACGGACCCAGGTGATGTCAGGGGTGGGGTGGGGCCTGCAGGA
GGGAATGGAGAGCCAGCACCTAGGGGAAGCTGGGAATTTAGGAAGATATCCAGAGAGTGTAACTTCAGTTCCACT
AATCTCACCTGGGTGAAAACCAGCCCTCTCCATGGATGATGGTGATTGCAGGTACTGACGATAGCTGCAGACTGC
TGGTGGTCTAGGCAAGCATGCAGGGATGGGAGCAGGCATTTCTGAGAGCTCCCCTTATCCCTGCCCCAAGACAAG
GTGGGGGCCCTGTGGGGAAGGACTATTTTATTCACTTCTGCATCTCCAGTTGTCTAACACATTGCTTATCACCTA
ACAGTCTCTTAATAGAAGCTTGCTATATTGAGCTGCCTTGAGTCCACATCATGCTGGTTGAACATAAGTAGAAAT
TGTGGGAGACTTAATCAGAGAAAATCTTTCATCTGTCTTACCTTACGTTCTAATGATCACTTCTGTGCCCACAAG
GACCATCTTTTCTGATTCATGTTCATGTCGATTTCTTTTTTATTTAACTTCCTCTTCAAAATTTCTGGCAGGATT
TCTTGGGAGCCAATTCTCCATTTTTACTTCCCCATGCCTCCCATTTTAATGACTGTAGGATTTTCTCAGGGTCTA
CTCAGCAAAACTTGTTAGTACAACATGAGCAAAACAGCAAATTTATGCAAACACTTAGCAAAGACTTAGCTGTCT
GCCTAATGCTAGGGTGAGACATGGGAGATTCACAAATGAAGAAACACAAGCATTGATCTCCAGGATCCTGCAGTG
GGAGGTATGCGGAGGACCAACCTGGGAACAGAGCAACGCAACACCATGTGATCTGTAATGAAAAAGAGGCCTGTA
CACAGCCAAGAGGTCACTGAGGAAGGAGCCATTGCTTCTGTGCAGTTAGTGCTGCGCTAGATTCTGCAGGGATGT
AAGAAATGTACCCCTATCAAACAAGAAAGACTATGTGAATTGCTGAATATGTGAGTGGAGTACCAGCACAATGCC
ATGGAGATGCAGACGTGCCCCATGTGGTGGGGCAGAGTCAGGAAGCACTTTAAGAAAGAAATAGCATTTCAGGTC
TTCCTTTAAAGGTAGAATTTCAACAAGAGGGTGCTCTGGAGAGTGTGTGTTACCCTGCTGAGAAAATCCTGGCGG
TCAGGTAAGATGCTACTGCCAGGGAAGATTGGCCAATTGATTGACTAAACCCTTAAAGGTTTGGGGATCTTGGGG
AGGATTCTGCTGGTGAGAGGGTCTGGACTTCCTCTTGGTCTGTCCACAGCTGGACCTTCTCAGCACACAAGAGAC
TATGAGGGTGACCATTTTGCACAGGACAGAATTCCAGCATGTTTTTCCCCTGGAGTGATGGAATGACCACCTGCT
CAACATCAGTGTCCTCACTGAGACCATGAGATTCAGTAGAGTGCTGGAAAGCTTCATGCTTACCTGTGTCTTCTT
AATGCTTAGTGTTATGATTGAAGGCTTCCTTCAGTCCTACCTTTTGTTCTGGGGTTCTAAGAATCTTAGGTGCAG
GCGAGGCACGGTGGCTCACACCTGTAATCCCAGCATTTTGGGAGGCTGACGTGGGTGGATGACGAGGTCAGGGGA
TTGAGACCATCCTGGCAAACATGGTGAAACCCCGTCTCTACTAAAAATACAAAAATTAGCCGGGTGTGGTTGCGT
GCACCTGTAGTCCCAGCTACTCAGGAGGCTGAGGCAGGAGAATCGCTTGAACCTGGGAGGCGGAGGTTGCAATGA
GCTGAGCTCACGCCACTACACTCCAGCCTGGGTGACAGAGCAAGACTCTGTCTTAAAAAAAAAAAATCTCAGGTG
CACCTGAGACAGATTGAATGTGGAAGGGGAAGTGAAACAGGCCTTCCAGGTGTGGGGCCTGGGTGCTGCTATAGT
TACAAATGGGGAAGTGAGACTATAGGTCTCAGTTACCTGTGGAAGGAAGGGTAGAGTGGAGTACTTACGCAAATT
AGCTAATTCTGGGAGCTTGGGGTGCTACCAGGGTATCAGGGAGAATACAGCCAGGGAATAGAATCTTCTTGAAGC
AAAGGCTGTTTGGAAGCCCCCAGAGTGGATGAAAAGGCTCAGTGGGAAACAACAGATATCAGGAGAGGGAGAAGA
AGATACCTATTTCTATACCTTTTGGCCTTGTGTTTTGCTTCAGACACTGTTCCCAGCAAGGTCAGTGGAACCCAC
TGCTCAAAACACACACTTGCTCCTTGTTCTGGTGTCATAATAGCTCTGCAAGCAGTGGTGGTGTTCAGCCTGGAG
AACGTTCCTTTTCTTTTTTTTTTTATCACAATAAACACTCATGGCTTCTCTGCTTCTTCCTTTCTTCTTTGTCTT
AGGACTCTTGAAAAACAGCTGCCAAATGTCAGTTTAGATATTTTGGAGGGAAAAAAGTTGGGAATCAATGTTTAC
AGGTTGCCTGCAATGTGCTGGAAACTACATAGTTGGTTCTTTTTAAACTTTCTCTGAATCCTGTCAGGAAAGTTC
CAGCAATCACATCTTAGTGGGTCCGGAATTCGTGGGTTCTTGGTCTCACTAACTTCAAGAATGAAGCTGTGGACC
CTCGTGGTGAGTGTTACAGTTCTTAAAGGTGGTGTGTCCAGAGTTTGTTCCTTCTGGTGTTGGACATGTTCGGAG
TTTCTTCCTTCTGGTGGGCTCGTGGTCTCGCTGGCTTCAGGAGTGAAGCTGCAGACCTTCACGGTGAGTGTTACA
GCTCTTAAGGCAGCGCGTCTGGAGTTGTTCGTTCCTCCCATCTGGAGTTGTTCGTTCCTCCTGGTGGGTTCATGG
TCTCACTGTGCTCAGGAGTTAAGCTGCAGACTTTCGTGGTGAGTGTTACAGCTCATAAAAGCACTGTGGACCCAA
AGAGTGAGCAGCAGCAAGATTTATTGCAAAGAGCAAAAGAACAAAGCTTCCCCAGTGTAGAAGTGTAGAACGGGA
CGCCAATGGGTTGCCAGTGTTGGCTCCCCCCAGCCTGCTTTTATTCCCTTATCTGGCCCCACCCACATCCTGCTG
ATTGGTTCATTTTACAGAGGGCTGATTGGTCTGTTTTACAGAGAGCTGATTGGTCCGTTTTGACAGGGTGCTGAT
TGGTGCATTTACAAACCTTGAGCTAGACACAAAGTGCTGATTGGTGTGTTTACAAACCTTGAGCTAGACATAGAG
TGCTGATTGGTGTATTTACAATCCCTTAGCTAGACATAAAGATTCTCTAAGTCCCTAGTAGATTAGCTAGACACA
GAGCACTGATTGGTGCATTTACAAACCTTGAGCTAGACACAGGGTGCTGATTGGTCCGTTTACAAACCTTGAGCC
AGACACAGAGTGCTGATTGGTGTATTTACAATCCCTTAGCTAGACATAAATGTTCTCCAAGTCCCCACTAGACTC
AGAAGCCCAGCTGGCTTCACCTAGCCGATTGTGCACCAAGTCAGCAGGCGGAGCTGCCTGCCAGTCACCTGCTAT
GCACCCGCACTCCTCAGCCCTTGGACGGTGGATGGGACGCCAGGGAGCAGGGCGCGGTGCTCGTCGGGGAGGCTC
CAGCGGCACAGGAGCCCACGGCAGGGAGGGTGGGGGGAGGCTCAGGCATGGTGGGCTACAGGTCCCAAGCCCTGC
CCCGCGGGGAGGCAGCTGAGGCCCAGCAAGAATTGGAGCGCAGCGCCAGTGGGCCAGCACTGCTGGGGGACTTGG
CACACCCTCCACAGCTGCTGGCCTGGGTGCTAAGCCCTTCACTGTCCGGGGCCTGCTGCGCTCGCCGGCCGCTCA
GAGTGCGGCCTGGGGAGCCCACGCCCACCTGGAACTCGCGCTGGCCCACGAGCGCCTCTCTCTCTACACCTCCGC
TCAAGCAGAGGGAGCCGACTCCGGCCTGGGCCAGCCCAGAGAGGGTCTCCCACAGTGCAGCTGTGGGCTGAAGGG
CTCCTCAAGCACGGCCAGAGTGGGCGCGCAGAGGCCGGGGGGCACTGAGAGCGAGCGAGGGCCACCAGCACGTTG
TCTCCTCTCATTAGGGTTGGGGAAATGGACCCTGAGAGAGATTAAGTAATTTGGTGATATTCTATAGTCACTCTG
GCTATGTAATTTATGGAGCCTAGTACAGAATGAAAATGTGGGGCTCATTTTTCAAAAAGCAGGAAACAGCTTTTC
CTTTCTTCCAGGGTCTCTTCCTCCACCTGCCATGCTGGTGTTTGGTTGCTATTTAATGTTGAGCCCTCTTGGGCA
CAGGGATACTTGCAGGGACAGGGTGTCTGCTCATTTTTCTGTAGACCTCAAAGGTGAGTCCTGAGGCTTCAGGGT
CACTGGCCTCCTTTTAGGGAGTCACGACGCCTTGTCTTTGTACTTCAGGAATGATTACGAATCTTTGTAGGTAAA
GCGGCAGAATGCCACGTCCTCTCCTGGTTGCCAGGACGTGTTCCTTGTGGTTTAATTGCCGGGTCTGCCCTGCAG
ACCCTGGCTGAGCGACAGATGAAAGGAGTACTCAGACACAGGTACGCAGTGAAAGAGCGGCTAGGGGACTGCCGA
AGAGTCAGCAGTCTCAATAAACTGGAGCTGCTCACTTTTATTCAGTACAGACATAATGCCGAAAGCCTGGAGCCA
ACGCAGTCTGTGGGTAATTAACATTGTTGTTCCGCCGTGCAGGGAGCAGTCTCGCCAGAGGATGATGAAAGGTTG
GTTTCCGGAAGTAAACAAGCTTATTTAGACAAACTCCCCTACATTCCCTTGTACCCACTCCTCGCCCTCTGCGTC
AGGGTAAGAGAACAGCTGCCTTCAGCTTATTCTCCCCCGAAGCTTTGCAGAGCCTTCTGACCTTTCAAAAGGTCT
TCTTCTTTCCCTATCGGTTCTCCCACTACTCTGACTGATCTCCTATATTTGATCTCACCTTAACAATCACTTCTT
AGAGCTGGGTCAGGAAGTATGCAGCATGCACCTGGCACTCCTAGTACTGTGCCCATGATGGGCATTGCTGATTGT
TCAGAGCATATTGGATGAGCCTGGTTCAGCCTCAGAATCTTCCACCCAGTGCACCATGGAGATGCTACCAATTGG
TTGGAGTTGCTCTGAGAGGTGACATTTCCTTGTGATTCTGCATTAGAAACATGTTGTTTGTCAGCCGAAACAGGG
AAACCTGACACGTTATCCGCCCCCAGGAAGATCCCATCATCATTCCATGCACCTTCAGTCCTGGGAGCTTACTTT
AAAAAAAAGTGACTGACATATGAGCGCAGGTCCCCAAACAGAGGGGAGGCAGGATGAGAAGCCAGATGAAGAGAG
TCAAGGTCCTGGGGCTGCTTAGCTTGGATGAATCTGATGGGAGGTGGGGTGCATCTGAGTGTTCTCTGCTGATGA
AGAACAGACTTGTTGCACGGGGGTAGGTGTGTGCTGTGTAAACACACATCAGAATCAGGACCCCGAATAGTGAAT
AGGCAAGAGTAACAGCTGAATTTGCCCAGCTCATCACAATTTAACATCAGTTTTCAAAAAGGTAAGAGCGTGGCT
TTCATAGCATGCAGAATCAACACACATCAAAGATTGATTTACTCATTTATGAAGGAATCAGCAAAATGACAAACT
TAGTTCAGAGAATATTTTGAGGCTCTGAGTAGATATAAAACTGGTTAATGTTTCTCAGGGCAATAAAAAGCTATA
AACGTTGGGGATTTCTTTTTTATCAGACAGAAATTATTTGCATACTTAACAGAAAAGATCTCCAAGTTACCATCT
AACTTCATAAGGTTCGAATAAAACTTCATAGAGTTATTAATGAATGGTAAATAGAAAAGACAAATATATGTTTTA
CCAGATAATTAAGTAATTCTTGGTAAACCTGGCAAACAGTACCCCAGTGTGACTCTGAAAAGACATGCTGCCCAT
CTTTTTGCCTTATTTCCACGTTTTAGGTATTTTTGTAAGATATCTATTCAATAAATATGTATTGAGCTCCTATGA
TGTCCCAGAAACTCTTTTAGATCCTGGAGATATAGCAGTAAACAAAACAGATGAAATTCTTGCTCACATGGAACT
TATATTCTAGTAGGGGAGACAGACATTGAAACAGAAAAATACATAGTATGGCAGATGGTGGAAAGTATTAAAAAG
AGTGCTGTGTAGTGTTTACAACTTACTCATTTATGAAGGAATCAGCAAGATGATAAACTCAGTTCAAAGAACATC
TTGAGGCACCGAGTACATTTAAAAGTGGTTAGTTTCTCAGGGCGATAAAAAGCCATAAACTTTGGGGATTTCTTT
TTTAGGTATGGAAACCTAAAGTAAAGAAGATGCTATGGTTTGCACATTTGTCCCCTCCAAAACTCATGTTTGAAA
TGTAATCCCAGAAGTGGCAGGATGAGAGATTGGCCCTTTAGGAGGTGACTGGGTCATGAGAGATCTGCCCTCATG
AATGGATTAATCCATTCATGGATTACTGATTAATACGCTAATGGGTTAATGGATCAATGGGTTATCCTTGGAATG
AAATGGCAGGCTTTACAAGGAGAGGAAAAGGGACTTGAGCTAGCATGCTCACCCTCCTCACCGTGTGATGCCCTG
TCCTGCCTCAGGACTCTGCGGAGTTCTGGTAAGCAAGAAGGCTGTCACCAGATGTGTCCCCTAAACCTTGGACTT
TTTGGCCTCCATAACTTTAAGGAATAAATTCATTTTTAAAATAAATTACCCAGCTTCAGGTATTCTGCTATAAGC
AACAGAAAATGACTAAAACAGGAGGCTTTACTGGAAGGTGTCCTCTTAGCAAAGACCTAAAGAAAGAGGGAGAGT
GAAACATAGAAATATCTGGGGAGAACATCCTAGGTAAAAGGAACAGCATGTGCAAAGGCCTTGAAAAGCAGCAAG
CCGCTCTCCCTCTCCCTCTCCCTCTCCCTCTCCCTCTCCCTCTCCCTCTCCCTCTCCCCCTCTCCCTCCCCCTCC
CCCTCCCTCTCCCTCTCTCTCCACGGTCTCCTTCCACGGTCTCCCTCTGATGCCGAGCCAAAGCTGGACGGTACT
GCTGCCATCTCGGCTCACTGCAACCTCCCTGCCTGATTCTCCTGCCTCAGCCTGCCGAGTGCCTGCGCACGCCGC
CACGCCTGACTGGTTTTCGTTTTTTTTTTTTGGTGGAGACGGGGTTTTGCTGTGTTGGCCGGGCTGGTCTCCAGC
TCCTGACCGCGAGTGATCCGCCGGCCTCGGCCTCCCGAGGTGCCGGGATTGCGGACGGAGTCTCGTTCACTCGGT
GCTCGGTGGTGCCCAGGCTGGAGTGCAGTGGCGTGATCTCGGCTCGCTACAACCTCCACCTCCCAGCCGCCTGCC
TTGGCCCCCCAAAGTGCCGAGATTGCAGCCTCTGCCCGGCCGCCACCCCGTCTGGGAAGTGAGGAGCGTCTCTGC
CTGGCCCCCCATCGTCTGGGATATGAGGAGCCTCTCTGCCTGGCTGCCCAGTCTGGAGGGTGAGGAGCGTCTCTG
CCCGGCCGCCATCCCATCTAGGAGGCGAGGAGCGCCTCTTCCCCGCCGCCATCCCATCTAGGAAGTGAGGAGCGT
CTCTGCCCGGCCGCCCATCGTCTGAGATGTGGGGAGCACCTCTGCCCCGCCGCCCTGTCTGGGATGTGAGGAGCG
CCTCTGCTGGGCCGCAACCCTGTCTGGGAGGTGAGGAGTGTCTCTGCCCGGCCGCCCCGTCTGAGAGGTGAGGAG
ACCCTCTGCCTGGCAACCGCCCCGTCTGAGAAGTGAGGAGCCCCTCCGTCCGGCGGCCACCCCGTCTGGGAAGTG
AGGAGCGTCTCCGCCCGGCAGCCACCCCGTCCGGGAGGGAGGTGGGGGGGGGTCAGCCCCCCGCCCGGCCAGCTG
CCCCGTCCGGGAGGTGAGGGGCTCCTCTGCCCGGCCGCCCCTACTGGGAAGTGAGGAGCCCCTCTGCCCGGCCAG
CCGCCCCGTCCGGGAGGGAGGTGGGGGGGTCAGCCCCCCGCCCGGCCAGCCGCCCAGTCCGGGAGGTGAGGGGCG
CCTCTGCCCGGCCGCCCGTACTGGGAAGTGAGGAGCCCCTCTGCCCGGCCAGCCACCCCGTCCGGGAGGGGGGAG
GGGGGGTCAGCCCCCTGCCCGGCCAGCCGCCCCGTCCGGGAGGGAGGTGGTGGGGGTCAGCCCCCCGCCCGGCCG
GCCGCCCCGTCCGGGAGGTGAGGGGCGCCTCTGCCCGTCCGCCCGTACTGGGAAGTGAGGACCCCTCTGCCCGGC
CAGCCGCCCCGTCCGGGAGGGAGGTGGGGGGGGGTCAGCCCCCCGCCCGGCCAGCCGCCCAGTCCGGGAGGGAGG
TGGGGGGATCAGCCCCCCGCCCGGCCAGCCGCCCCGTCCGGGAGGGAGGTGGGGGGGTCAGCCCCCCGCCCGGCC
AGCCGCCCCGTCCGGGAGGGAGGTGGGGGGGTCAGCCCCCTGCCCGGCCAGCCGCCCCGTCCGGGAGGGAGGTGG
GGGGATCAGCCCCCCGCCTGGCCAGCCGCCCCGTCCGGGAGGTGAGGGGCGCCTCTGCCCGGCCGCCCCTACTGG
GAAGTGAGGATCCCTCTGCCCGGCCAGCCGCCCCGTCCGGGAGGGAGGTGGGAGGGTCAGCCCCCCGCCCGGCCA
GCCGCCCTATCCAGGAGGTGAGGGGCGCCTCTGCCCGGCCGCCCCTACTGGGAAGTGAGGAGCCCCTCTGCCCGG
CCAGGACCCCGTCTGGGAGGTGTGCCCAGCGGCTCATTGGGGATGGGCCATGATGACAATGGCGGTTTTGTGGAA
TAGAAAGGCGGGAAGGGTGGGGAAAAAATTGAGAAATCGGATGGTTGCCGGGTCTGTGTGGATAGAAGTAGACAT
GGGAGACTTTTCATTTTGTTCTGTACTAAGAAAAATTCTTCTGCCTTGGGATCCTGTTGATCTGTGACCTTATCC
CCAACCCTGTGCTCTCTGAAACATGTGCTGTGTCCACTCAGGGTTAAATGGATTAAGGGCGGTGCAAGATGTGCT
TTGTTAAACAGATGCTTGAAGGCAGCATGCTCGTTAAGAGTCATCACCACTCCCTAATCTCAAGTACCCAGGGAC
ACAAACACTGCGGAAGGCCGCAGGGTCCTCTGCCTAGGAAAACCAGAGACCTTTGTTCACTTGTTTATCTGCTGA
CCTTCCCTCCACTATTGTCCTATGACCCTGCCAAATCCCCCTCTGCGAGAAACACCCAAGAATGATCAATAAAAA
TAAAATAAAATAAAAAAAAGGAATGAATCAAGAAAAAAAAAGAAAAGAAAAGAAAAGCAGCAAGCCAGCCAGTGT
GTTTGGAATGTTCTCTTATGGAAAATTTCAAAGATATGTAAAACTAGGGCTAATAATACAATTAACCCCTACTTA
CCCATTACCCAACTTCAACAACTATCAACATTCTGCTGTTCTTATTTCATCTATTTACCCATTAAAAAAAAAGTG
TACTTTAAAGCGAATTCCAGAGTTTGTATAATTTTGTCTGTAAATCTTTCAGTCTGTATCTCTAAATCTTTCAGT
CTGTAACTCTAAAAAGAACATTAAAAAACACAACTATCATACCATCATCCTACCTGACACAACTGAGTAATTTTT
CATATCATCCAATATCGATCAGAAGTTTAATTTTCCATGATTTTCTTTAAAATGTAGTTTTATAATTGATTTGTT
CTAATCAAGATTTGCACATTGCATTAAAAATATATGTATCTTAAATTTCCTTTAATTTACAACCATTTCCCTCCC
CCATTTAAGAGTGCATATTAATTTATTAAACAGACTGGGCAATTCATCTTGTAGAATTTCCCACCTTCTGGGTTT
GGCCAATTACATCCTTGTGGTGTTATTTAAAATCCTCCTCTATTCCCTTTATTATCTGTTGACTGACAGCTTGGC
CAATCAGAATCACTTGAACGAGCTGATTACACCCCCTCTTTCTGAAACGTTTTCTTCCCTTGAGTCTGTACACAC
TGTTGGCTTCCAAATTTATATCTCCAACCCAGACCTCTCTCCAGTACTTCTGCTTGCTACTGTGTTTTTCATAAC
TTTCTGATTTTTTTAGTATCCAATGCTAGAAAATATATCCATTTTGAGAAAGAGACAAAGTATGAGCTCATATTG
ATAATTTCATTTCAAAGTAAAGGGAACAAAGTTTTTATTTAACATGTTAATTTTATGCTTGTTTCTCTTTTACAC
TGAAAATCTTAGTTCTCAATGACATTAATATAATTATGTATTTACTTCCCATATATATGTTGTATATAATTGTTT
TATATATACATATGTGCATATATATTATTGCTAATGAAACGTCTACTGAATGATGTAAGTTTTCTCTGTGATTCT
TTTGGTCCTTGGGACACAGGACTTATCCCACTAGTGATGTGTAGTCAAAATACTATGTACCAGTGTTTGATACCT
TAATTAAAGGATCTCTCTGGCTGCTGCATTGAGAACAGATGGTAGCAGGACAAGGACATCAGCCTGGAAGCCATC
TGGAAGCTCTTTAGTGATTCAGATAAGATTTGATTTTGGCTTGGATGGTGATGGATGATGTTGAGAAGTGGGTGG
ATTCTGGATATGTTTTGAAAGTAGGTTCCATGTGATTTGCCATGGGCTAATATATGGAATGTGAGAGAAAAAGAA
GAAACAACAATGCTTCCAAGATTTTGGGGCAGAGGTACTGAAAAAATGAATTTCCATTTATCAGAAAGAGAAAGA
CTGTGGTAAAGCAAGTTGGGAAGGAAACAGCAGCTCAGTTTTCCACATTCAGTAACCCTCCCTTATCTAGGGTTT
CACTTTCTGCAGTTTCACTTATCCATGGCCAGCCACAGTCTGAAAATATTAAATGGAATATTCCAGAATTAAACA
ACTCGTAAGTTTTAAATTGAGCACTCTTCTGAGTAGCGTGATGAAATCTCATGATGTCCTGCTGTCTTCTGCCCT
GGATGTGAATCATCCCTTTGTCCAGCATATCCATGCTGCATGTTGTACCTGCCTGTTACTTGCCTAAGCAAGTGG
ATGGCTACTTGCTTAGTGTCCATCTCAGTTATCAGATTGAAAAAGCAGTACATATACATAGGGTTTAGTACTATC
TGCAGTTTCAGGCATCACTGAGGGGGTTGGAACATAACCCTGGCAGATAAGGGAGACTACTGTTTTAGTGGAGAT
GTTGACCAGAAGACTGGTTCATATGAATATGGGGGTCCTGGAGAGAGGTCTGGGCTGGAGATATAAATTTGGAAA
TCAACAGCGTATACAGACACAAGGAAAGAAAACATTTCAGAAAGAGGGGGTATAATCAGCTTGTTCAAGTGATTC
TGATTGGCCAAGTAAGATGAAGATTGGAAATTGACCAATGGATCGGTGACTTGGCAAGGTCAGTTTGGGAGGAGT
GGTAGTGATGAAAGCTTATGTGGAACAAATTCAAGAAAAAAACAGAAGAGAGAGGAATTAGAGATTGTTGTGCAC
AGACAACTCTTGCAAGAGGTTTTGCTGGCCAGGAGAACAGAAATACAAGTGAAGTAGTGGCTGGATGTTTTCCAA
AACATGAAACTTAGTTTTCATAGGAAAAAAATGGTTTTTCTTTTTCTACTTATTCAATTTTGTGCACAATTTCAT
TACATTATATAAGTAAAAACCACAAGCACGAACATGTTTATAAATAGGTAAATAAATAACAAAGTAGATAGAAAC
AAAAATTCTCAGGTGTGCAAAAGAGTAGTTTATTAGCTGTGTAGAAGACAGAAAACTTGCTTTTATAGAGGGAAT
GAATAGTGTTGATTAGTATAGTGAGTCAGTTAAATAGGTATCAGTTGAATTTTTTGAGACAATAAGTACATTTAG
AATTGGCTAGGCATTATTCTTCCATTAAAGGAAACTCTTAGTAATAGATTAGGTCAGAAAACCACAAAGACAGTT
TTCTTGAATAAGGGGGTGGTGGGAAAAAATGGATGTAAGATGCTGAGAAAAAACAGGAAACTCAACAATAGATGA
AATCAAGAATTAGTTTCGTGTGTGTGTGTGTGTGTATTTCACAGAAAAGGGCTGAAAGGGGAGGAATGGTTATAT
TCACAGATTTTTGTAGTTGATCTTAATAGGGAATAGGAGATTACCTTTTTTTCAGCAAAATATTACCAGCAAATG
TCTACTCTGGAAATAGAGGATAAGAATCTTCTATTTAAAGACCAAAAAGGTGAAAACTGAGGTCAGAAATTTAAA
GAGTAAATGTAGCAAGATGCTAACATTCTTGTTCCCTCTAATAACTCTTCTTTTTTAAAAATTATAAAAGTAAAA
CATGCCCATTGAAGAATATTTGGATGAATACAAAAATATAGAAAAGAATGTAAAATCGCTTAAGCCCCCAAGTAG
AATTCTAAATAGCGGAGAAACCCACGATTTAAAAAAATAAATAAGGAGATCCACAAAAGGAAGATATATACCTGC
TTAAGTGGACGGCCTCAGCATAAGTAGTCTTACAGTTACAATTAATTTTTTTTCTATTATACGATGTTTTAAAAA
ATTGCTAAGCTCAGTTGTCTACTCCCTGCTAGAATGTATCAAACATGCTGCATCCACATAGTAGACAGTTATTCT
ACATTTTTGGTCTTTGGATGTTTTGAAGCAATTAGCAAAGTTTGATTTGAAGAGACATTATAAATTTCCGACGGC
ATATTTTTCTCTGGCCATGATCCATATTTGCCTTGATATTGTCCAAATGTTCATTTTATCTTATGACTATCAAGC
AAATATTATAAAGTTATTTGTGAATTTGGCACTTCATAGATGAAATATTGTGAAATTCTGAATAAAACTGCTTCA
TTGATTATCTTGGTTCCTAAAAGAGATAGTTTATGAGTTAAATTATCTCTAACGTTGTCTAAGTTGGCAGTAATT
AAATCTCCATGGGAATTCTTAATAAAGGCAAAGAAGGACTGAAAACACCTTGCTGAAACTGAGAGAGATTTAAAA
CCACAAAAAAAATGTAAGATTTAGTTAGTTGTTCACGTTTTGGGGTCCTGCTTTTATGTGTTAATACTTCCAGAT
AAAATTTTTTCAAAGGTAGTTTTGAACCCCAGGATCTGAATATATTAATTTGTTTATCTCTGAAAACTGTATACT
TTGTGTTTCCCAGGTCTGGCTTTCTTCATTTTTCCTACTTGAGGGAACTCCTTTTCTTCCTTCAATATCCTCATA
TCCTCCTTCAATGTTTCTCAGTCAATATCTCCTCTCAATTTTTCCATTTAAAAATTAAAAATTAAATTATTAACA
TTTTAAATACACTGAAATGTTCATGGAATAGTAGTACAAATACCCATTTACCCACCACATGGAGTTAATAGACAA
TAAAAATTTGCCATATTTGTTTTACATCTTTTTTCTAGTTTTTAAAGAAATAACATGTTACAAATAAAATCGAAA
TACTCTTTCCGCCGATTTTCTTTTCTCCTGTCTCAGAGGCAAATACTGCTACTTGCTTCTCTTGTATCTTTTTAG
AAATATTCTCTGCATATATAAGCATATATCCATATATTTTTCCACACATAGTATCTCTTCCGTGTCTTAGTTTTT
TTCATTCTACAATGTATCTTGATGATCAGTGTAGCTTTGTTTCATTCTAATAATTATATAGATTCCACTTTACAA
ATTTACTATACTTTGGACTTGTATCTATGAACGTTTCGATTGTTTTAAACCTTTGGCAACATTACAGACAATGCT
GCAATGAAATCCTTCTGTATGTATACATACGTGGATGTGTGTATAAGATATAATTCTGGAAGTAGAATTGCTAAG
TTAAAGCATATGCTACTTAATTTTGAAAAAATTGTCGAACTTCTCTTCACAAGGCAATTATCTTCTTGAGGTAAT
GAGAACTCCTATTTCCCCATACCCATACTACCCAGAGTATCTTCAAATTTTCTGATTTTTGCTGGTCTGATAAGG
GAACGTTACCTCAGTTTAATCTTTATTTGCATTTACTATGTGTTTTTGCCCTGTAGAGTGAATTTTACTTTTCTA
CCTAACAATATATTTTTGTGATTTTAATGACATTACTTTTGAGATTTACCCATGCTAATATATAAACTCTGGTTC
TGCAATTTTAAGTGCTATATAGTATCCTATTGAATGACCATACTACTCATTTATTTATTATTATCTACTTGTTAA
TTAGTCTGTTATCTTTTTTGTCAGTTTTTGTGGGCATAAACAATGCTAAAGCATACATGTCTACTTGCGCATATG
ATTTTTTCCCAGTGTGTTTAATAGAATGACTAAGGTAGAATGACTAAAGTTCTGAGGTGTGGCAGGTATGCCATC
TTCACCTTTACTGGGTATGCTAGTGTGCTGCAGCTGCCGTGATAAAGTACTACAAGCCGGGTGGCTTCAACAATA
GCATTGTACTGTCTCACAGTTCTGGAGGCTGGAAGTCTAAAATCAAGGTGTCAGAAGGGTCGGTGCCTTCTGAAG
GTGTGAGAGAGAATCTGCTTCGTGCCCTCTTCCAAGCTTCTAGTAACCTCAGGTGTTCCTTGTCTTGTTGATGGT
GTTGTCCCAGTGTCTTCATATTGTCTTCTCTCTGTTTGTGTTTGTGTTTGTCTCTATGCCCAAATTTCCCCTTTT
CATAAGGACCCAGTCGTATGGGATTAGGGCTTACCCTAATGATCTCATCTTAACTTGATCATCTGTAAAACCTTA
TTTCCAAATAAGGTCATTTTCTGAGGTACTGGGTGTTAGGATTTCAACATCGTTTGGGGGGGTAAAATTCAATCA
ATAACAACAGGTATACCAGTTAAGATGTTTTTGGCTGCAACTAACAGAACATTCAACTGAAAAGGTTTAAAATAT
ATTGTAAATTTTAAACAAATGTTTTATAGAGATGAGTTCTGACTGTGTTGCCCAGGCTGGTCTCAAAATCCTGGC
CTCAAGCAATCCTTCCACCTTGGCCTCCCAAAGTGCTGGGATTACAGGCATGAGCCACAGAGCCTGGCCACTTTA
AAATGTTGTTGACACACATAACAAGACATCCAGAAGTGGGGTGGTCATAGGATCGGTTCAGCAGCACAGTGATGG
CCGAGGCTCTTTCCTTCCACTCTTCCATCTCCCTTGAGTGGCTTTCAGCTTCTTGTCACAAGATTGCTGCATTAT
GTCCTCATATACCTGGAACAGAGGGCAGGGAGAGGGGCAAGAGCACACCCCCATTTCAAGTCTCTTTTCATCCCA
AAGTAACATATTTCCCAGCAGCCCTCTTTCAACCCTCTCTAGGCTTCTCCTTAAATTTCACTGGCCAGAATTGGG
CCACACGGTTACCCAAAGGTATAAAAGAGGCTTGAGAAGTGAGAATTTGATATTTTGAATCATAATAGGAGACAT
GCTTTGCCAATAGGAAAGAAGGGTAAAAGAAATCTTCTGGAGAGTACGGAACCAACGGCGTCTGCCACGTTGCAT
ATGGCCAAATGGTCACCAAATTTGTTGTATCACTTTATTTTCACAAAATTGGTGTAAAGTCCTCATTGCTATTCA
TCCTCGTCATCATTCGGAATTCTCAAATTTAAAAAATGCCCATTTCCTTGTTGTTTTATTTTTTTAATTCCCTGA
TCATTAACATAAGTAATTATCTTTTTATGTGTTTATTTGTCATTCAGGTTTCTTCTGTGAAAAGCCTATTTCTGT
CTTTTGCTTGTTCTTATGTTGAATTGTCTCTACACATTCAGGATTCTAATCTTCTGTCAGTTATGTGGATTGCAA
ATAAAGTCTCCCAGTCTATGGCTTAACTCTTGACACTATATTTATCACGTCCCTTATTTACTAAAGATTTAAATA
TGATCAAATTTATGTTTTCTTTTAGGGTTGTGCCTTTTGTATCTTATTTAAGAACTGATTTCTTTAATGTGAGAT
CACAAAAGGGTTATAAATATTCTAAAAATGTTAAACTTTTGCTTTTCATAGTTAGATCTTTGATCCACTTGGAAT
TTATCTTTGTGTGTGGTGAAATTAGGAAGCCCATTTCATTTTTCCCCAGATGGATAGTCAGTTGTGTAAGTGCTG
TTTATCAAATAATCCATCCTTTATCGAGGGTTTTCTGATGCCACTTCTGTTGCATCTTGTGTTTCTTTATGTATG
TCAGTTTATTTCTGGACTCTCCAATCTGTTTTATGGTCTGTTTGTCGCTATACCACTGCCACGTTGTTTAAATTA
CTAATGCTTTGATATCCAATAGAACATGTCTGCTTTCTGTTACGGGAGATAAATTTACATTTTTAAGAAGTTTAT
ATAAATGGAATACATATCATTTACTCTTTTGTGTCTGAGTTGTATGTCTTTGTCATGATGTTTTTGTGATTCATC
CATGTTGTATGTATTAATAGCTTGTTCCTTTTTATTATTGGGTCTATTGTGTGTATATATCACAATTTGTTTATT
TATCTATTGATAGACATTTGGCTTGTAGTCACTTTTTGGATACAATGAATAGAGGTTCTATGAACATTAGTGTAC
AAGTTATTGTATGGACATACTGACAGAGCAGGAGCACAGTCCTCTTGGACAAACACTGCCACTTTAAGTTCCAGC
TCCATTTTTAGCCTCATGCATCTCAGGGAAATCACTTCTCTTCTAACTACAAGTAGCCAGAAAGAGCAAACAGTA
AACCACAGATAAAACAGCTCAGGCACAGAGGGAGGAGGGAGAAAAGTCTCTTGGGTAACTGCCACACTTCACCCT
CATACAGTGGGCCCCAGTAAAACAGTGGGCGTTAATAAACACATTATTTTCCCTTCAGGTGCACTAAAATAGGGA
AGCTAAAAGCAGACTCGGGGGGTATGCCTGCAGCTGCAGAAAAATGTATAAAAACAGACACACAACTCTCCCTCC
AAAATAAGCACAACAAAAAACACAAAAGCAGTCCAAGCCTCTAATAAACTCTCCTATCCTAAATCCTTAAAAACT
CTTAGTCTGTAAGAGAGTGTGCTGTTGACCTAGCTCAGCCAAAAGCTCCTCACAGGTTCGTTTTCTCTAAAATAA
ACCTGTCTTAACTGGCAAGCCACCTTTCGTGTTTTTTTTCCTCTTTCTTTAATTCTTACACATACTGTTTTATTT
CTCTTGAGTGAACACCCAGAAATAGAATAGCAGAGCCATATGGTACATAAGTTGATTAGCTTTTTGAGAAACCAC
CAAACTGTTTTATAAGGCAATTGTATAGTTTTACATGTGTAGCATCAGTGTGTGAATATTCTAGTTGTTCTACGT
CCTTGTTAACATTTGGTATTGTCAGACTTTTAAATTTTAGCCATCTAAAAATTTATAGTGTTATTTTATGGTGGT
TATAGTTTGCATTTCCCCCATGACTAATGATGCTGAGGATCATCTCATAGGCTTTTTGAAGTATGTGTTCAAATC
TTTTGCCCATCTTTAAAAATTAGGGTTTTTGTTTTGTACAAATACTTGGAAATTAAGCAACATACTCCAGAATGA
CCAATGGGTCAATGAAGAAATTAAGAAAAATAAAAAAACTTACTGAAAATGATGAAAACATGTCTAACAAATAAA
AATTGATACACAACATACCAAAATCTATGGAATACAGTAAAAGCAGTACTAGGAGGAAAGTTCATAGTAATGATT
GCCTACGTCAAAAAAGTAGAAAGATTTAAAACAACTTAACAGTGAACCTCAGGAAACTATAAAAGCAAAACAACA
ACAACAAAACCCCCAAACTCCAAATTAGTAGAAGGAAGTAAATAATAAAGATCAGAACAGAAATAAATGAAATAG
GTTGGAAAAGTAATACAAAAGATCAACAAAATGAAAAGTTGTTTTTTAAAAAAAATTGACTAAGCATTACCTAGA
CTAACTAAGAAAAAAGAGGGAAGAACCAAATAAATGAAAAAGGAGATGTTACAATTGATACCACAAAAATATAAA
GGATCGTAAGAGACTATTATGAACACCAATAAATTGGAAAGCCCAGAGGAGATGGATAAATTTCTGGGCACCTAC
AACCTACCAAGATTGAACCAGGATGAGATACAAAATCCGAATAGACCAATAACAATTATTGAGGAACCTCAATAA
TAATTTTTATTAAACAACAATAAAAAGTTTCCCAATTAAAAAAAAAAGCTCAGGACTGGATGGCTTTACTGCTGG
ATTCTACCAAACTTTGAAAAATAACTACCAATTCTTCTCAAACTATTCCAAAAAATTGAAGGGAAGAGAATTCTT
CCAAACTCATTCTATAAGGCCAGAATTAACCTGATACAAAACCAGACAAGGATACAACAACAAAAAAAGAATTTT
GCAGGCCAGTATCCCTGATGAACATAAATGGAAAGTTCCTCAACAAAATACTAGCAAACTGAATCCAACAGCACA
TTAATAAGTTTATTTACTAAAACCAGGTGGGATTCATTCCAGGGATGCAAGAGTGGTTCAACATATGCAAATCAA
CAAACATAATACATCCCATCAACGGAATGAAGGACAAAAATCATATGATCACCACAATAGATGCAGAAAAACAGT
TGATAAAATTCAACATCCCTCCATGATAAAAACTCTCAAACAATTAGGTTTAGAAGAAGGAACACACTTCATCTT
AATAAAGGCCATATATGACAAATCCACAGCTAATATTGTACCAAACAGGGAAATGTTGGAAGTTTTTTCTCTAAA
AACTGGAACAAGATAAGGATGCTTACCCTCACTACTCTGATTCCACATAGTACTGGAAGTTCTAGCCAGAGCAAT
TAGGCAACAGAAAGAAATAAAAGACATCCAAATTTGGAAGGAATAAGTCAAATTGACCATGTTTGCAGATGACAT
CCTCTTACCTACAGAAAAATCTAAAGACTCCACCAAAAAACTCTTAGAATTGATATACAAATTCAGTAAAGTTGT
GAGATACAAAATCAACATACAAAAATCAGTAGCATTTCTATACACCAATAATAAACTATCTGTAAAAGGAACCCC
ACTTACAATAGCTACCCCCCAAAAAAAACCTCCACCTAGGAGTAAATTTAACCAAAGAGGTGAAAGATCTCTAGA
ATAAAGACTACAGAACACTAATAAAAGAAATTGAAGAGGACATAAAAAATTGGATAGATATCCCATGTTCATGGA
TTGGAAAAATTAATATTGTTAAGATTCCATACTACCCAAAGGAATCTACAGATTCAGTGCAATCTCTATCGAATT
TTCAATGGCATTTTTCACAGAAATGGAAAAAAAGATTCTTAAATTTGTTAGGAACCATAAAAGACCCCAAATAGC
CAAAGCAATTGTTTGTTTTATTTTATTTTATTTTATTTTATTTTATTTTATTTTATTTTATTTTATTTTATTTTA
TTTCACTTCATTTCATTTTATTTTTGAGACAGTCTCCCTCTGTCACACAGGTTGGAGTGCAGTTGCATGATCTCA
GCTCACTGCAATCTCTCCCTCCTGGGTTCAAGCAATTCTCCTGCTTCAGCCACCTCAGTAGCTAGAATTACAGAC
ATGCACTACCATGCCCTATTTTTAGTAGAGATAGGGTTTCACCATGTTGGCCAGGCTGGTCTCAAACTCCTGACC
TCAAGTGATCCACCTGCCTCAGCCTCCCAAAGTGCTGGTATTACAGGCATGAGCCTCTGCTCCTAGCCAAGCCAA
AGCAATTCTGAGCAAAAAGAACTGGAGGGATCACACTACTTTACTTTAAAATATAATACTATAGTAAAACAGCAT
TGTATTGGCATTAAAGCAACACATAAATCAATGGAACAGAATAGAGAACCCAGAAATAAATTCACATATTTATGG
CCTATTGATTTTCTGCAACAGCACTAAGAACATAAACTGAAGAAAGGACACCTTTTTCAATAAATATTGCTGGGG
AAACTGGATATTCATATGCAGAAGAATGAAACTAGAGCCCCATCTATCATAATATAAAAAAGTAACTCAAAACGA
ATCAAAGACTTAAGTGTAAGACCCCAAATTATGAAACTACTAAGAGAAAACATAAGGAAAATGTTCTGAGCAAAG
ATTTTATGGATAAGACCTCCAAAACACAGTCAACAAAGGCAAAAATAGACTAATGGGATTACATCAAACTAAACA
TTTCTGTGCAGCAAAGTAAACAATCAACAGAGTCATGTGACAAACTACAGAATGGGATAAAATATTTGCAAACGG
TTTCTCTGACAAGGAATTAATATCTAGAATATACAAGGAACTTAACAGCAAAAAGCCAAATAATCTGATTTTTAA
AATCGGCAATTGATCTGAACAGACGTTTCTCAAAAGAAGACATAAAATGGCCACTAAGTATTTGAAACAGTGCTC
TATGTGACTAATGATTAGGGAAATGCAAATCAAAATCACAGTGAGATATTATCTCACGCCAGCTAGAATGGCTAT
CATCAAAAAGAAGAAAAAATAACAAAGCTAGCAAGAATGCAGATAAAAGGAAACTTAAAACATTTCAGCTTTTAC
TTTAGATTCAGGGGTTACATGTGCAGGTGTATTGCATGATGTTGAGGTTTCAGAATATGATTGAACCCATCTCCC
AGGTGGTGAGCATAGTACCCAATATGTGGTTTTGCAACCCTTCCTTCCTCCTTCCCTCCCTCCTCTTATACTCCC
CAGTGCCTAGCATTCCTATTTTTATGTCCATGCGTACCCAATGTTTAGCTCCCACTTATAAGTGAGAAATGTAGT
ATTTGGTTTTCTGTTTCTGCGTTAACTTGTTTAGGATAATGGCCTCCAGCTGCATTCATGTTGCTGCAAAATACA
TGATTTCATTCTTCTTTTTGTGGCTGCATGGTATTCCATGGTGTATACATACCACCACATTTTCTTTATCCAATC
TGCCATTATTGGGCATCTAGGTTGATTCCATGTCTTTGCTACTGTGAATAGTGCTGTAATGAACATATAAGTGCA
TGTCTTTTTTTTGGTAGAACAATTTATTTTCCTGTGGGCCATATACCCAGTAGTGGGATTTCTGGGTTGAATGGT
AATTCAGTTTTTATTAATAGTTCTTTAAGAAATCTCCAAAGTGATATTCACAGCGGTTGAACTAATTTACATTCC
CACCAACAGTGTATAAGCGACAAAGAAAATTCTCACACATTGTTGGTAGGAATGTAAATTAGTACAGCTATTATG
GAAAGCAGTGTGGAAGTTCCTTAACAGGCTAAAAATAGAACTACCATATGATCCAGTAATCTCGCTACTGGACCT
ATATCCAAAGAAAATAAAATCATATGTCGAAGAGATACCTGCACTCCCATCTTTACTGTAGTTTCATTTATAATA
ATGAAGATATGGAATCCACCGAAGTGTCTATCAACAGATGAAGAGATAAAGAAAATGTGGGATATATAGACAATG
GAATGCAGCCATAAAAGAGAATGAAATCCTATCATTGGTGGCAACATGGATGAGCCTGGAGGACATTATGTTAAG
TGATATAAGCCAGGCACAGAAAGACAAGTTTCATATATTCTCACTTACATGTGGGAGCTAAAAAAGTTGATCTCA
GAGAAGTAGAGAGTAGAATAGTGGTTACTAGAAGCTGAGAAGGGTAGGGAGACAGAGATTGATCAATGAATACAA
AATTATATATATGGATAGAGGAAATAAGTTTTAGTTTTAGTGTTCTATAGCATTGTAGGGTGACTATAGTGAACA
ATAACTTATTGTATATTTTCAAGTACTGAGAGGAGAAAATTTTGTACATTACCAGCACAAAGAAATGATAAACCT
TTGAGATAATGGATATGCCAATTACACAGATTCGATCATTATGCATTGTATGCACGTATTGAAATGTCACTTCAC
CCCATAAATATGTGCAATTACATGTCATTTAAAAGTGATAAGAAAAATTAGTTTTTTTGATCTTATTATTGACTC
GTAGGAAGCTTATATATTCTGAAAAGAAGTCCATTTTCAGATGTGTACTAAAAATATTTTCTTTCATTTTATGAC
TTACCTTTCCATTTTCTTTAATGGAAACTTTTAAAAACTTTTAAAAAACAAAGTTTTAAAAAAATCTAGTTAAGG
TCCAGTTTGTTAACTTTTTTCTAATATGGCTTGTGACGCTTATTCCTTCTGCCTAGAATGTTCCTGGGATGTTTG
TGGAGCTAAGTCCTCGCTTCCTTCAATTCTTTACTCAAATATGTCCACCCTATTTAATGTCAACTGTCCACCGTA
TTTAATGCCACCCTATTTAATATCACTAACACCTCCTCCCCCCTCACTCTTGACATTCATTCTAGTCTATTTTAC
ATTTTTTTCTCATAGAACTCATAAATTTCTAGCATGCTTTATAACTTACATATTCATTATGTTTATTGTTTATTG
TCCGTCTTTGTTCCAGTAAAATGTAAACTCCTAGAAGAACAGAGACCTGTGTTTTGTTCACTGATGTACCCTAAG
TGCTCACAAGTGTTTCTAGCACCTAGTATTTGCTCAATAAATATTTGCTAGGTTGATGAATTAATGATTTCTAAG
CTTTCCTTCAGCCTGAAGAGTTTTCTGATTGTAAGATTCTACTTAGATAATCCTAATTGTCTCAGTGACTCTCAC
CAGTCACTCACTTCTCCCACAAGGTGGCAGTCTTTACCTTCAACACAGGTTCTGGTAGCCTCAAATTTGAGAATT
AATAGCTGAGTTAACCTGCTTGTTTTCTTTGAGCCCAGACAGCCTGCCCTATGGGAACTGACAGCTGTAAAATTT
AAAGGACGAGTGTAATTACCCTGCAAGATCTGAGTGCTTTTAGGCAAGAGGATTTAGGGGGTGAGAGTTTTCCTG
GAGAGGGACACATTATGAAGGTGATATTGCTTAATTGATGGGGACTTTGAAACATAGTTGCTCTTTGTGAGAATG
GTATAGGTTTAGAGAGAGGTGCTAGCACAGAGCTGTGACACCTGAAGTAGGCTGACCGCAGACAAATTGGATTTA
ACCACCAAATATATCTGTGTTTTCATGTCTTCCTGCCCCGTGCCCTCTTATCTGACTCACTTTACCCCAGCACTG
GGGAATAACTGTGCCCTATTCTGGTCCTGACCCTTTTGTACCATCTAGGGAAATGAGAACTCCTCTTGGGGTCTC
AGATCCTCATTTCTGTTAGAACCAATCCTATTCTGTGGGTAGGGCCATGGTTGTAAATTTCCTGTGGGAGGCAGC
ATTGCTTTGCAAAAAGAACACAGTTTGGCATGTGAGGCAGCTCTGCCACTTGGACAAGGTGATAACGCTTTAGTC
TCTTTATTTCTAAAACAGGGAAGATGCTAATACCCCGCCCATGGACTAGTATGAGATTTAAATGGCAGGTACTTG
GCACAGTGGCAGGTGGTGAATGCTCTTTGGTGATCATGACTATCCCTTTCTCCTGGTAGTGCTGCCTCCTCCCTC
TGAGCACCTGGAGTCAATCCACCTTGGGTAGGTCAGAGAAGGCAGAAGAAAGTGGTGGGAGGTGAACTCGACGGA
ATGATGTACAGGGCGATAGGGTGAGTGAGAGGTCTGGGATCTATTGGCAGGAGCAGAATGGTAGGAAAGGGAAAA
CATGCCATTGACCTTGAATCTTGACATTTGTGCCCATCCTATGCTGTGTTGAGCCTCAGGTCACCGTTTGCGGAG
GTGAGCAGAAAACTGCTAACAGATCGAGGCTTCTCCAGCCTTCTAGGTAAACTTTCATCAGTGGGTTAGTTGTCT
TGTTCAGAAGCTGATCACGGAGCTTTGGCCAAGCATAAACACTGATTATGGCAGTCCAATTGTCATAATCCCTTT
GATTCTTTAATATCACCTTCAAGATTGTTTGTTATTGTCAATGCCCCCACAACCTAAGACCACCAGGAACACACT
GTAATTGAAAAAGGTGGGTTTGTTGCTCTCTGCAAGAAGGGAGGACACTCAGCGTAGAGACTCATGAGGGTGGGG
CAGGTTTTATCCGATGACGTTAGAAAGGACTTACTGAGGATTTGGGCTTGTTTTAGGAGATTTGGGGGAAAGGTT
CAAGGAGACTGGCTTTTCCTGGATGCTGCCAGGAAGTAGTGGGATGGTAGTAAGTCTGTGGTAGGATGTTTAAAT
AAATTTCCTCTACTGGGCTGGAAGAATGAGAAGGCTGAAGCTGTAATAAGTAAAGAAGTGGCAGTCACTCCTATC
AGCTATGATAAAAGGATGTTTGGCTATTACTTTATGGTTTGGATGCTATTTTTGCTTGTGTTCACATCATGGTCT
ATCATGGTGATAGGCCACGTACACAGTGGCCTTGTCTGATGCTGGTGTCCCATGGAGTTGATTATGCTCAGCTGA
AGGACACTAAGGCCCAACTGTGGGGGCCAGGCCAGCTCCTGAGTGTCAGGGGGGGCTGCACTGCTTTGTCATTAT
CAACATCTCCACATACAATACAGCCTGTGCATGTGAGGTCCCAGAAGGAAGGAGCTAAAGCAGCTAGACTGGGAT
CACTTTACTCAATTTGAGGAGAGGAGTGCTCTTGAGGGAACCAGAGGAAGGCAGATGAGCTGGTCTGATCTCCTT
TTCTATTGGAGCTCTATGCAGATAGACTGAAAATATTTGCTAAAACAAAGAGCTCCATCTCTAGAATACCCTTAG
CAGGATGTCCTTGATTAAAGGATTATTTCTGAAAACTAAATCCAGAATCCGTGAGGCATGATTCCCTGGAAGATC
ATGTAAGCTGTACAATTCTCTATGGAATAAATTGGAGACTTCATCCCTTAGATCCCTTTGACTGTGTGAGGAACC
CCACGAAACCTCACTTAGTGACTTTATTCATTCTTCTGGGCCTGAAAGCATATGTCTCATGCAGGAAGGAAGGCA
GGACCAGTGGGGCTTTGCAGGTTGTGCCATTCTCTCCTTGCTTTGCTTGAGACTCTTTATTACCTGTCTCCCTGA
CATTATTTGTAGTGTGATTCTGGGTGTGTTCTGTGATTCTTGTGACTAGGTATCCAATTTTGCTTAAGGATGCAA
GGAAGTGTTTGGGGAGAAAGCTCTATTGGAAGAGGTCTGTAGTCCTAGCCTCCCTCCCCACCCCACATTTCACAT
CATTAGACCTCAGCACATGGGTCTGGGGCACCAACACTGTCTTACCTGTTACACAGTGTGGTCTTTATCTGGATG
AGGGATGCGAAAGGATACATTGTGACCAAGAGACCTGGGAGAGGCACAAAAATAACAGGTGACCACCAAGGGTGC
TTGGACCTGAGATGTTTCCATTTCCTAAGACCCTCCAAGATTCTCCAACATTTGGTATAGTTGCCCAGGCAATTT
AACAAGAAAATCAAATTTTTGTTATGACCCCCATTGTAATTTATGCTTATCGCAGAAAAATTGAGACTATAAGAA
GGAGAATAGAAGGTCACAAAACCACTCTATACTAGTCCAGGGATAGCTATTCTTACAACATGGATTGATCAGTGT
GGGGTGATTTCTCCTAGTGTTTTTTGGACAGAGAAGCATTGAAGATGCCCTGGTTTTAAGGTCTTAGGATGAAGG
AATTATAGTTGAACAGTTCAAAATGATGTTATGAATTACTTTCAGATTTGTTTGCTTGATTGCATTAGCCTTGCC
TGGCCCTACGGTAACTATTTGGTTCCATCATGGTGGCTGAGTAGGTGGCTCTGGAAAAAGAGCTATTCAAGAAAA
GCTTTTCTTTCTCTAAAAATATTGTAGGGGGCTCGCCCTCTGTTCTTGGAAGCAACGTTTGGGATGGCCTCTTGG
GAGGCTGTCTGGTGAAGTGTCTAGGGGTATGTGGTCTGGACTTGGACAGGACAAGATGCAAATTCTGGCTGGGAT
ATTCTAGTTGTGGAGTGTTGGGCAAGTTACTTGGTCTTCTGAGTTTATAGGTAAACTGGAGATAATAGGTATGTG
TGAATGAGGATCCAATGAGATGCCTGTAAAACACTTAGCCAGATGGCTGCGTGAAGAAAGCACTTGGTAAATGCT
AATTGTTGTGGTTGTTATAATTAGTACAATGATTAGTCATTGCTGATTGTTGGCTAACTGGCGGTAAGAAATGAA
AGTAAAGTAAGGCAGTAGCAGCTGAGGGAGGTGGTGGAGGGAATCAGGAGACACTTGGAGGTTCTGGTTCTGCCT
GAGTTTAAGTGCTGGGGAAAACTAGTTGAATAACTGCTGGTCTAACATTTAACAGCTGTGTGACCTCGGGCTAGT
CACATTTCCTTTATAAACTTCCTTTTTCTCATTTGCGAAATGAAGGGGTTTCGTTAGGTTACTTCTCATCACCCC
TGGTTGACCATTAGAATCGTTTGGGAGACCTTTTTAGAAATTCTTGGTTCTGGGGTCTTCCATTTTCCCCATTCT
CACTTGGTGGGTCTGAAAGCAGGCACTGCAGCTTTTCCAAAGCTCTTCAGGTGACCTTGAAGTGTGGCCAGGGCT
GAGAACCTCTGACTTCCAACAGCACTTCTGGTTTAGGAAGGAGCAAATCACCGGCACAGAATGAGCTCTCAGGAA
CGGCTGCTGAGCTAGTAATTGCCGTGACACTGTCTCCCTGTCCCAACTGCAGGCACCCCTAGACGTCTCCTGATG
AAGACTTCCAATTTTGGAACAGAAGAATCTTTGAAAAAAATATTATTGAACTTCCAGAAATGATTCATTCCTTCT
CTGCTCCTTCTTTAGTTGGAAAGATCTGCCCCCATCCCTGTCTACTGCAGTCCCAATCCCTTTTTATTTCAACAT
ATATATCCAAACCAACAAAAAAATTGACTCGCACAACCAAGGTGAGGTGTTTGGCTTTAAGGATAAAATAAATAG
TTTCATAAAACCTGCCCCCAGATTTCTCATTGCCTCTACTCATTTTCTCTAATTTGTAGGGCACACTGAAAGCTC
GGATTCATAAGATGTAGAAAGGGCAGAGAGTTAAGTTACAAACTCTTCCTAGGTCTGTTTCAACTCTAACGTTCT
ATAGCTCTGCTCCGTCTAAACAAGGAATTCTGTTAAATTTGTAACCTGGACTTTCTTGAATACTGAGGTAATGTT
TCTTGAAGTAGGATATATATACCCAAGAAGAATAAAAATAATTCTGGAGGTGTCTTAATTCTCTGTGGGACTCAA
TAAAAGTTTTGGTGATTATATATAAACACACTTATGAAAGCATCTGGTACATGTAGGTGCTCAGTGCACATGAAT
TTCTCTTTCCTGCCAGATCTTTTGTAGTGGAAAATTATCTTATTCTTCCATCTTTGTCTGCAAAGATGCTGCTAA
GGAAAGATGTAGAAGAGTTTTACAAGGGAGTTGGAGTATGGGAACAGGAAGTTCCCAAGAAGGCCACTGCATTAA
TAGAATTGAAACAAGATCCCCTAAAGGAAAATCGCTGCCAAATCTCTTTTCTCTAAACTATCCAAAATGGTGCCC
CATAAATTTTCATTGACATTGAAGTACATAATGTAATAAGCTTTTTTTTCCTTAAATATATAATGTACGGAGAAA
ACCAGGTTGATAATGGTTTCCTGGCATAGCTTTCAAAGGCAAGTGTGGATGAAGTGGAAATATGGTGCACAGATA
TTGGAAAGAAACCGCTGTTGAACTCTTCACATTTTCATGTATAACCCAATGATTCTCAAACCTAAACTTGCATCA
GAATTGCCTGGAGGGCTTATTAAAACATGATTGTTGGATGCCACCCTTGCATTAATTAATTACACCTGCAACTGT
TCTATTTTTAAATGGTCACATTTTAAGGTACTGGGCTGAGGACTTCATTATGTGAGTTTCAAATGGGACATAATT
CAACCCCTAGCCCAATCCTAGTGAGTGGTGAGTGGTATCTCTTCGTTTTGATTTCTACTTCCCTAGTGACTAATG
ATGTTGAGCATTTTTATGTGTGTTTATTAGCCATTTTTATATCTCTTATGAAAAAATGTGTATAAAATTATTGGC
CCATTTGTTAATTGGGTTATCCATTTATTATTGAATTATAAGAGCTCTTTACTACTCTGGATGCAAGTCCTGAAA
CAGAAATAATATTTACAAATATTTTAATCCATTCTGTGAATTTTATTTTCACTTTCTTGATGGCGTCCTTTGAAG
AACGTAGGATTTTAATTTTGATAAAGTGTAATTTATGTATTTTTTCTTTTGTTGCTGTGCTTTTGGTATCATATT
TAAGAAATAATTGCCTAATCCAAGGTTATGACAATTTTTTATTCTATGTTTGCCTCTAAGAATTTTCTAATTTTA
CCTTTTATATTTAGGTCTTTCATTCATTTTGAGTCAATTTTTGTATATGGTGTAAAGTATGGGTCCTAATTTATT
CTTTTGCATGTGGATATCTTGTTGTCCCTGCACCATTTGTTGAAAAGTGTTGTTTTTTTTTTCCCATTGAATGGC
CTTGGCACTCTTGTCTAAAATTAATTGATGGTAACTGTAAGACTTTATTTCTGGACTCTTTATTCCATTGATCTA
TATTTCTATCATTGTTACTGAGCAATGTGCTTGCTGCCTGACAGATAGGGAAGCCAATATTATGGAACTGGTTTT
TGAGAAAAGCAAAAGCTTTATCGTGAGGTTGACTTGCAAGGAAACAGGATGCAAAGCTCAAATCTGTCTCCCCTT
CTGGGATCTGGGACAAGTTTTATGGGTTAGGGAGGGCAAGCTGGTATGCAGAAGCACTGGTAGGGCAGGTTTCAA
CTGGAAGTACTTTAAACAAGACCATTTATGGTAAGGTATGGTAAGGGTCTTAACACTGGACATGCCTGGGCTCAG
GTTTCTTGCTTTTAAAAATGTTTGGGCCCTCAGGTTCCAGTCATGTCTTGACCATTTTCTTCTGTGGTGGGGCAG
GAGAGGAATTTTTCTTCTGGGTGTTATTCAAGGTTGAGGTCTTCTTTTCTGCATTGCTTCGGCTGCATGACTTAA
CAACTTTTTGACTTTGTGCCTGTTAAATAACTTGACATACTATTATCATCAGAGTAGGGCCAGTTAGAACTGGTC
CTGTGATTACATCATTATGCCAGTACCAATTATCTTGATTACTGTAGCATTGTAGTAAGTTTTGAAATCAGGAAG
TTTGTGTCTTTCAACTTTGGTCTTCTTTTTCAGGATTTTTGGCTCTTCTGTGTTCCTTACATTTCCATATGAATT
TTAAGTTAAACTGTCACTATCTGCAAAAGAAGGAACTGGGATTTTTATAGAGATTACATTGAAGCTGTAAATCAG
CTTGGAGAATACTGTCATCTTAACAATATTAAGTCTTCTGGCCGGGCACGGTGGCTCACGCCTGTAATCCCAGCA
CTTTGGGAGGCCGAGGCGGGTGGATCACGAGCTCAGAAGTTCGAGACCAGCCTGGCCAACCTGGTAAAACCCCGT
CTCTACTAAAAATAATAATAATAAAAAACTGGGCATGGTGGCATGTGCCTGTAATCCCAGCTACTCAGGAGGCTG
AGGCAGGAGAATCATTTGAACCCTGGAGGCAGAGGTTGCAGTGAGCCGAGATCGCACCATTGCACTCTAGCCTGG
GCAACAGGGCGAGATTCTGTCTCAAAAAAACAAAAACAAAAACAATATTAAGTCTCCTGATCCATGAATGTAGAA
TGTTTTTCCATTTGTTCAGGTCTTCTTTACTTTGTAACAGTGTTGTGTATTTTTCAATGTTCCAGTCCTGTAATT
CTTTGTTATATTTACTCCTAAGAATATTAATTGTTTTGCTGCTATTATAAGTGGAATTGTTTAAATTTTGATTTT
ATATTTTTCATTGATAGTATATTTTTCATTGATAGTATACAATTGATTTTTGTACACTGATTTTGTAACCTGAAA
CCTTGCTGACCATGTTTACTCGTTCTAACAGTTTCCTTTTTGTGGATTTCTTATAATTTTCTATATACAGTATTT
CATGTCATCCATGAAGGGGATAGGTTTACTTCTTCTTATCTAATCTGGATGAGTTTAGTTTATTTTTCTTACCTA
AATTCCTTGGCTAGAACTCCAATACAATGTTGAATATAAGTAATGAAATCAGACATCTTTGGACTGTACTTGATT
TTAAGGGGGAGCATCCAGTCTTTTGCCATTATGTATAATGTTAGCTGTGGGGTTTAATAGATGAATTTTATCAGG
TTGAGGAAATTTTATTTCTAATCTGCTCAGTGTTTTTTTCATCACAAGAGTGTTGGATTTTGTTAATATTTTTGT
GTGTCTATTGAGATGATCATATGGTTTTTGTCATTCTACAAAATACAGCACATTAAATTGATGGATTTTTACATG
TTAATTTTTTTTTAAATTTTACTTTAAGTTCTGGGACACATGTGCAGAACGTGCAGGTTTGTTACATAGGTATAC
ATGTGCCATGGTGGTTTGCTGCACCTATCAACCTATCATCTAGGTTTTAAGCCCTACATGCATTAGGTATTTGTC
CTAATACTCTCCCTCCCCTTGCTCCCCACCCCCGCCGACAGGCCCCGGTGTGTGTTGTTCCCCTCCCTGTGTCCA
TGTGTTCTCACTGTTCAACTCTCACTTATGAGTGAGAAGACGTGGTGTCTGGTTTTCTGTTCCTGTGTTTTTTAG
CTGAGAATGATGGCTTCCAGCTTCATCCATGTCCCTGCAAAGGACATAAACTCATTCTTTTTTATGACTGCATAG
TATTCCATGGTGTATATGTGCCACATTTTCTTTATTCAGTCCATCATTTATGGGCATTTGGGTTGGCTCTAAGTC
TTTGCTATTGTAAATAGTGCTCCAATAAACATATGTGTGGATGTGTCTTTATAGTACAATGATTTATACTCCTTT
GGGTATATACCCAGTAATGGGATTGCTGGGTCAAATGATATTTCTGGATCTAGATCCTTGAGGAATCGCCACACT
ATCTTCCACAGTGGTTGAACTAATTCACACTCCCACCAACAGTGTAAAAGCATTCCTATTTCTCCACAGCCTCAC
CAGCATCTGTTGTTTCCTGACTTTTTAATGATCGTCATTCTAACTGGCGTGAGATGGTATCCATTGCGATTTTGA
TTTGCATTTCTCTAATGACCAGTGATGATAAGCTTTTTTTCATATGTTTGCTGGGCACATAAATGTCTTCTTTTG
AGAAGCATCTGTTAATACCCTTCGCCCACTTTTTGATGGGGTTGTTTTTTTCTTGTAAATTTGTTTAAGTTGTAG
ACTTAGGATATTAGATCTTTGTCAGGTGGATAGATTGCAAAAAATTTCTCCCATTCTGTAGGTTGCCTGTTCACT
CTGATGGTAGTTTCTTTTGGTGTGCAGTATCTCTTTAGTTTAATTAGATCCCATTTGTCAATTTTGGCTTTTGTT
GCCATTGCTTTTGGTGTTTTAGTCATGAAGTCTTTGCCCATGCCTATGTCCTGAATGGTATTGCCTAGATTTTCG
TCTAGGGTTTTTATGGTTTTAGGTTTTACATTTAAGTGTTTAATCCATCTTGAGTTAATTTTTGTATAAGGTGTA
AAGAAGGGGTCCAGTTTTTGTTTTCTGTATATGGCTAGCCAGTTTTCCCAGCACTATTAATTAAATAGGTAATCC
TTTCTCCATTGCTTGCTTTTGTCAGGTTTGTTGAAGATCAGGTGGTTGTAGACATGTGGTATTATTTCTGAGGTC
TCTGTTCTGTTTTTGTTTTTTGTTTTTTGTTTTTTGTTTTTTTTTTTTGAGATGAGATCTCGCTCTGTTACCCAG
GCTGGAGTGCAGTGGCACGATCTCGGCTCACTGCAACCTCCGCCTCCCTGGTTCAAGCAATTCTCCTACCTCAGC
CTCCTGAGTAGCTGGGATTACAGGCATGTATCACCGCGCCTGGCTAATTTTTGTATTTTTAGTAGAGATGGGGTT
TCACCATGTTGGTCAGGCTTGTCTCGAACTTATCACCTCATGATCTGCCTGCCTCAGCCTCCCAAAGTGCTGGGA
TTACAGGCGTGAGCCACCGTGCCCGGCCAAGGTCTCCGTTCTCTTTCATTGGTCTATATATCTGTTTTGGTACTA
GTACTGTAGTTACTGTAGCCTTGTAGTACACTTTGTAGTCAGGTAACGTGATGCCTCCAACTTCGTTCTTTTTGC
TTAGGATTGTCTTGGCTATACGGGCTCTTTTTTGGTTCCATATGAAATTTAAAGTAGTTTTTTTCTAATTCTATG
AAGAAAGTCAATGGTATCTTGATGGGAATAGCATTGAATCTATCAATTACTTTGGGCAATATGGCCATTTTCACA
ATATTTATTCTTCCTATCTATGAGCATGGAATTTTTTCTATTTGTTTGTGTCCTTTATTTCCTTGAGCAGTGGTT
TGTAGTTCTCCTTGAAGAGGTCCTTATGTCTCTTGTAAGTTGTATTCCTAGGTATTTTATTCTCTTTGTAGCAAT
TTTGAATGGGAGTTCACTCGTGATTTGGCTCTCTGCTTGTCTATTATTGGTATATAGGAATGCTTGTGATTTTTG
CACACTGATTTTATATCCTGAGACTTTGCTGAAGTTGCTTATCAGCTTAAGGAGGTTTTGGGCTGAGACGTTGGG
GTTTTCTAAATATACAATCATGTCATCTGCACACAGAGACAATTTGACTTTCTCTCTTCCTATATGAGTACACTT
TATTTATTTCTTATGTCTGATTGCCCTGGCCAGAACTTCCAATACTATGTTGAACAGGAGTGGTGAGAGAGGACA
TCCTTGTCTCGTGCCACTTTTCGATAGGAATGCTTCCAGCTTTTGCCCATTTAGTATGATATGGGCTATGGGTTT
TTCAGAAATAGCTCTTATTATTTTGAGATATGTTCCATCGATACCTAGTTTATTGAGAGTTTTTAGCATGAAGGG
ATGTTGAATTTTATTGAAGGACTTTTCTGCATCTATTGAGATAATCATGTGATTTTTTTCATTGGTTCTGATTAT
GTGATGGATTATGTTTATTGATTTGTGTATGTTGAACCAGCCTTGCATCCCAGAGATGAAGCCAACTTGATCGTG
GTGGATAAGCTTTTTGATGTGCTGCTGGATTCAGTTTGCCAGTATTTTAGTGAGGATTTTTGCATCGATGTTCAT
CAGGGATATTGGACTGAAATTTTCTTTTTTTATTGTGTCTCTGCCAGGTTTCGGTATTAGGATGATATTGGCCTC
ATAAAATGACTTATGGAGGAGTCCCTCTTTTTCTATTGTTTGCAATAGTGTCAGAAGGAATGGTACCAGCTCCTC
TTTGTACCCCTGGTAGACTGCATGTTAGACGAGATAATATGTATGAACTACCTGGCATATAATAGATGCTTCCTA
AATAAGATTCTAAAAAATAATTATGCTCCAAAAATATTTTTAAAATCAAATAATTTATGTTTTATTTTCTGTGTT
TTATCTCAGACATGTAGACTGCCAAAGTGTATGGGATGCTTTCAAGGGTGCATTTATTTCAAAACATCCTTGCAA
CATTACTGAAGAAGACTATCAGCCACTAATGAAGTTGGGAACTCAGACCGTACCTTGCAACAAGGTAATTGGGGG
CATGCCATTGATTTTAAAACTGGGGATAAAAGCCAATGGTAACAATTCATAGGTCCAAATTTTTATTAGAATGAA
GGAAGAGGAAAAATCCAGACATTATAGTGTGAGTGTGGTTGGTAGGAATGGAATTTGCAGGCCATTGAGGGGCCA
TGATATAATTAAGATTTAGGACATCTGGAGAAGGGAGCTAAGAGAGAGAAATAGGGATACAGAGATAGGAAAGGG
GCTTTGGCCAAAAACTAGGCAGAAAAAACCTAACACCAAACCCAACTCGAACAAACAAATTAACACGACCTATAT
AATAACAAAACTTTCCCCTGACCTATGATAATAATAGTAGTAGTAGTAGTAATAACAGCAATGCCAAGTTACACT
TGCAGACTGCTTCTTCTTTTTCTTGCTTACAAAAGACTCTCCTAATCCTTACTTTCTTAGGCCTTCATAGCCATT
CTCTGGAATGGGCACATCAGGTGTCAGCATCCCAATTTCACCAGTGAGAAAACTGAGGGTTGGTGTGTTTAGGTG
ACCAGTGTTGCCCAAGTTTGACAGGCTTCAAAGTGACCAGTTTAAATGTAAATGGTATGAGACCTGGAGCCACAG
AGGCCTGGATTCTAATACATTGGTTATATTGGAAAAGCTCTATCAGAGTGCACCTTTTCTATAGCCAATGTTTAA
GGCAAAATTCCATGTGCCTAAAATTTTCTTTGTGAAGCCCTTAAATCCATCCAGAAATTACAGCCTCTCATTCCA
TTGTTAGTGAGCTGGAGTCATTGTGAAACTTCTCCATTCACTAGGCGTGATGCCCTATGCAGAGAAGGTGTTTGG
CAAATAATAACCCAGGCTGACATTTGTCAAATAAGTGACTATGCGATGGATAGTATGCTAAGCAATTTACTTGCA
TTTATCTCAGTTAATTTCCCTAGCACCCCATTAGTTTATTTCAGTCATTATCATTACCATTTTACAGGTGTAGAA
AGTGGGGCTTAGTGATGTTTTGGTTGCTCAAGGTGAAACACCTGATAAGTGATGATGATGCTGGGCTTCAATAAG
GGCTGGGATTTTAGGGCCCATACTTTAAACCAGTATCCTTCACTGACTCCCATTAAGAATGAATAGGGGGAGGAG
CCAAGATGGCTGAATAGGAACAGCTCCAGTCTGCAGCTCCCAGTGAGACCAACGCAGAAGGTGGGTGATTTCTGC
ATTTCCATCTGAGATCAGGTTTCCTCGTGTGTCTACACCACCAGGGCCCTGGGTTTCAGGCACAAAACCGAGCCG
CTGTTTGGGCAGACACCAAGCTAGGTGCAGGAGTTTTTTTCGTACCCCAGTGGCGCCTGAAACCCCAGTGAGACA
GAACTGTTCACTCCCCTGGAAAGGGGGCTGAAGCCAGGGAGCCAAGTGGTCTCGCTCAGCGGGTCTCACTCCCAC
GGAGACCAGCAAGCTAAGAACCACTGGCTTGAAATTCTTGCTGCCAGCACAGCAGTCTGAAGTTGACCTGGGATG
ATGGAGCTGGGTGGGGGGAGGGGCGTCCGCCATTACTGAGGCTTTAATAGGCGGTTTTCCCCTGACAGTGCTAAG
GGGGCTGGGAAGTCTGGACTGAGTGTGGCAACGTGGTTGTGGCCAGACTGCTTCTCTAGATTCCTCCTCACTGGG
CAGGGCATCTCTGAAGGAAAGGTAACAACCCCAGTCAGGGGCTTACAGACAAAACCTCCGTCTCCCTGGGACAGA
GCACCTGGCAGAAGGGGCAGCTGTGGGCACAGCTTCAGTGGATTTAATCATTCCTGCCTGCTGGCTCTGAAGACA
GCAGCTGATCCTGACAAGAGGGATTCTCCCAGCACAGCACACCAACTCTGCTAAAGGACGGATTGCCTCCTCAAG
TGAGTCCCTGACCCCTGTGTCTCCTGACTGAGAGAGACCACCCAACAGGGGTCGATAGACACCTCATACAGGAGA
GCTCCGGCTGGCATCAGGCCGGTGCCCCTCTGGAATGAAGCTTCCAGAGGAAGGAGCAGGCTGTCATCTTTGCTG
TTCTGTAGCCTCCACTCGTGATACCTTCAGGTGCGGGAGGAACCCAGGTGAATAGGGTCTGGAGTGGACCCCCTG
CACACTGCAGCAGCCCTATGGAAGAAAGGGCCTGACTGCTAAAAGAAAAAACAGAAAGCAACAACATCAATGAAA
AAGACCCCACAAAAACCCATCCAAAGGTCAGTAGCCTCAAAGATCAAAGGTAGATAAATGCAAGAAGATGAGAAA
GAATCAGCACAAAAATGCTGAAAACTCAAAAAGCCAGTGTGCCTCTTCTCCTCCAGATGATCTTAACACATCTCC
AACAAGGGCATAGAACTGGGCTGAGGCCCCTAAAAAGAGATGAGTTCATGTCCTTTGCAGGCATATGGATGAAGC
TGGAAACCATCATTCTCAGCAAACTATCACAAGATCAGAAAACCAAACACCACATGTTCTCACTCATAAGTGGGA
GTTGAACAGTGAGAACACATGGACACAGGGAGGGGAACATCACACACCAGGGCCTGTCAGGGGTGGGTGCTAGGG
GAGGATAACATTAGGAGAAATACCTAACGTAGGTGACGGGTTGATGGGTGCAGCAAACCACCATGGCATGTGTAT
ACCTATGTAACAAAACTGCACATTCTGCACATGTAACCCAGAACTTAAAGTATAAAAAAACAAAAGATACTAGCT
ACATTTACCCAATGTTAAAAAAAAAAAAAGAACTGGGCTGAGGCTGAGGTGGATGAATTGACAGAAGTAGGCTTC
AGAAGATGCATAATAATGAAATTCACTGAGCTGAAGGAGTATATTCTAACCCACTGCAAAGAAGCTAAGAACCAT
GATAAAACATAGGAGCTGTTAACCAGAATAACTGGTTTAGAGAGGAACATAAATGACCTGATGGAGCTGAAAAAC
ACAACACGAGAACTTCAAGATGTAAACACAAGTATCAATAACCAAATAGACCAAACAGAAGAAAGGATATCAGAG
CTTGAAGAGTATCTTGCTGAAATAAGACAGGCAGACAAGATTAGAGAAAAAAGAATGAAAAGGAACAAACAAAAC
CTCTGAGAACTATGGGATTACATAAAAAGAACCTATGACTGATTGGGGTACCTGAAAGAGACAGGAAGAATGAAA
CCACGTTGGAAAACACACTTCAGGATATCATCCAGGAGAACTTCTTCAACCTAGCAAGATGGGCCAACATTCAAA
TTCAGGAAATCCAGAGAACCCCAGTAAGATACTCCATGAGAAGATCAACCCCAAGACACATAATCATCAGATTCT
CCAGGTCACCTATAAAGGGAAGCCAATTAGACTAACAGCAGACCTCTCAGCAGAAACCTACAAGCCAGAAGAGAT
TGGGGGCCAATATTCAACATTCTTAAAGAAAATAATTTCCAACCTTGAATTTCATATCTAGCCAAACTAAGTTCA
TAAATGAAGGAGAAATAAAATCTTTTTCAGACAAGCAAATGCTAAGGGAATTCGTCACCACCAGGCCTGCCTTGC
AAGAGCTCCTGAAGGAAGCACTAAATATGGAAAGGAAAAACCATTATCAGCCACTACAGAAACACACCGAAGTAC
ACAGACCAATGACACTATGAAGCAACTACGTAAACAAATCTTCACAATAACCAGCTAGCATCATGATAACTGGAT
CAAATTCACACATAACAAATTAACCTTAAGTGTAAATGGGCTAAATGTCCCAACTAAAAGACATGGAATGGCAAG
CTGGATAGTCAAGATCAATTGGTGTGCTGTATACAAGAGACCCATCTCACATGCAAAGACACACATAGGCTCAAA
ATAAGGGATGGAGGAATATTTACCAAGCAAATGGGAAACAGAAAAGAGCAGGGGTTGCAATCCTAGTTTATGACA
AAACAGACTTTAAACCAACAAAGATCAAAAAAGAAAAAGAAGGGTATTACATAAGGATAAAGGGGTAAATTCAAC
AAGAAGAGCAAACTATCTTAAATATATATGTGCCCAATACAGGAACACCGAGATTCATAAAACAAGTTCTTAGAG
ACCTTCAAAGAGATTTAGATACCCACACAATAATAGTGGGAGAATTTAACATCCCACTGTCAATATTAGACAGAT
CATCAAGACAGAAAATTAGCAAAGATATTCACGACCTGAACTCAGCTCAGGATCAAGTGGACCTGATGGATATCT
ACTGAAGTCTCCATGCCAAAGCAACAGAATATACATTATTATTGGTGCCACATGGCATCTACTCTAAAATTGATC
ACACAATTGGAAGTAAATTACTCCTCAGCAAATGCAGAAGAACTAAAATCATAACAAACAATCTCACAGACCACA
GCACAATCAAATTAGAACTCAAGATTAAGAAACTCACTGAAAACCATGCAATTACATGGAAATTGAACAACCTGC
TCCTGAATGACTCCTGGGTAAATAATAAAATTAAGCCAGAAATTAAGAAGTTCTTTGAAACTAATAGGAAAAAAG
AGACAATGTATCAGAATCTCTGGGATGCAACTAAAGCAGTGTTAAGAGGGAAATTTATAGCACTAAATGCCCACA
TCAAAAAGCTAGGAAGATATCAAATTGACATCCTAACATCACAACTAAAAGAACTAGAGAACCAAGAGAAAACAA
ATCCCAAAGCTAGCAGAAGACAAGAAATAACCAAGCTCAGAGCAGAACTGAAGGAGATAGAGACACAAAAATCCC
TTCCAAAAAAAAATGAATGCAGGAGGTGGTTTTTTGAAAAAAAATTAATAGAATAGATGGATCGCTAGCTAGACT
AATAAAGAAAATAGAGAAGAATCAGATAGATACAATAAAATGATAAAGGGGATATCACCACAGAAATACAAACAA
CCATCAGAGAATACTATAAATACCTCTATGCAAATAAACTAGAACATCTAGAAGAAATGAATAAATTTCTGGATA
CATACACCCTCCCAAGACTGAACCAGGAAGAAGTTGAGTTCCTGAACAGACCAATAACAAGTTCTATAATTGAGG
CAGTAATAAATACCAACCAAAAAAAAAAAAAAAAAAGCCCAGGATCAGACAGATTTATAACTGAATTTTACCAGA
TTTACAAAGAGGAGCTGATACCCTTTCTTCTGAAACTGTTCCAAAAAATTGAAAAGTAAGGACTCCTCCCTAACT
CATTTTATGAGACTAGCACCATCCTGATAATAAAAACTGGCAGAGATTTAAAAAAAAAAAGAAAGAAAGAAAACT
TCAGGCCAATATCCTGAAGAACATCGATACAAAAATTCTCAACAAAATACTGGCAAACTGAATCCAGCAGCACAT
CAAAAAGCTTATCCACCATGATCAAGTTGGCTTCATCCTCAGGATGCAAGGCAGGTTCAACGTACATGAATCAAT
AAATGTAATTCATTACATAAAGAGAACTAAAGACAAAAACCACATGATTATCTCAATAGATGCGGAAAAGGCCTT
CGATAAAATTCACCATCCCTTCACGTTAAAAACTCTCAATAAGCTAGGTATCAAAGGAACATACCTCAAAATAAT
AAGAACCATTTATGACAAACCCACAAGCAATATCATACTGAGTGGGCAAAAGCTGGAAGCATTCCCCTTGAAAAC
CGGCACAAGACAAGGATGTCCTCTCTCACCACTCCTATTCAACATAGTATTGGATGTTCTGACCGGGACAATCAG
GCAAGAGAAAGAAATAAAGTCTTTTCAAATGGAAAAAAGGAAATAAAATTGTCTTTGTTTGCAGATGACATGATC
CTATAACTAGAAAACCGGATCATCTCAGCCCCAAAGCTTCTTAAGCTGATAAGCAACTTCAGCAAAGTCTCAGGA
TACAAAATCAATGTGCAAAAATCACAAGCATTCCTGTACACCAACAACACGCAAGCAGAGAGCCAAATCATGAAT
GAACTCCCATTCACAAAGGGAATAAAATACCTAGGAATACAGCAAACAAGGGAAGTGAAGGACCTCTTCATGGAT
ACCTATAATCCACTGCTCAAGGAAATCAGAAAGGACACAAACAAATAGAAAAACATTCCTTCCTCATGGATAGGA
AGAATCAATATCGTGAAAATGGCCATACTGCCCAAGGTAATTTATAGATTCAGTGCTATTCCCATTAAACTACTA
TTGACATTCTTCATAGAATTAGAAGAAACTATTTTAAAATTCATATGGAACCAAAAAAGCTCATATAGCCAAGAT
GATCCTAAGCAAAAAGAACAAAGCTGGAGGCATCGTGCTACCCAACTCCAAACTGCACTACAAGGCTACAGATGC
CAAAATAGCATGGTACTTGTACAAAAATAAACACATAGACCAATAGAACAGAGTAGAGATCTCAGAAATAAAACT
ACACATCTGCAGCCATCTAATCTTTGGCAAACCTGACAAAAACAAGCAATGGGGAAAGGAATCCACATTTAATAA
CTGGTGCTTGAGAACTACCTAGCCATATGCAGACAATTGAAACTGGACCCCTTCCTTGCAACTCATACAAAAATT
AAGATGAATTAGAGACTTAAATGTATAACCCAAAACTATAAAAACCTTAGAAGAAAATCTAGGCAATATCATTTG
GGACACAGGCACAGGCAAAGATTTCATGAAATTGCCAAATGCAATTGTAACAAAAGCAAAAATTGACAAATGGGA
TCTAATTAAACTAAAGTGCTTCTGCACAGCAGAAGAAACTATCATCAGAGTGAACAGAAAACCTGCAGAATGGGA
GAAGATTTTTGCAATCTATCCCTCTGACAAAGGTCTAATATCCAGAATTTACAAAGAACTTAAACAAATTTACAA
GAAAAAAATAAACAGCCCCATCAAAAAGTGGGCAAAGAACATGAACAGACACTTCTCAAAAGAAGACATCCATGT
GGCCAACAAACATATGAAAAAAAGCTCAACATCACTGGTCATTAGAGAAATGCAAATCAAAACCACAATCTCATG
CCAGTCAGAATGGCATTATTAAAAAGTCAAGAAACAGCAGATGCTGGTGAGATTGTGGAGAGATAGAAATGCTTT
TACACTGTTGGTGGGAATGTAAATTAGTTCAACCATTGTGGAAGATAGTGTGGCAATTCCTCAAAGATCTAGAAC
TAGAAATACCGTTTGACCCAGCAATCCCATTACTGGGTATAATAGAAATCATTCTATTATAAAGATATGTGCATG
CATATGTTCATTGCAGTGCCATTCACAATAGCAAAGACATGGAATCAACTCAAATGCCCATCAGTGATAGGCTGG
ATAAAGAAAATGTGGTACGTATACACCATGAAATATTATGCAGCCATAAAAAGGAACAAGATCATGTCCTTTGTA
GGGACATAGATGGAGCCAGAAGCCACATCTTCAGCAAACTAACACAGGAACATGCAAATGCTGCATGTTCTCACT
TATAAGTGGGAGCTGAACAGTGAGAACACATGGACACCAGGAGGGGAAAAACACACACTGTAGCTTGTTGGGGTT
GGGGTGAGGGGAGTGAGAACATTAGGACAAATAGCTAATGCATACTTGGCTTAATACCTAGGTGATGGGTTAATA
GGTGCAGCAAACCCATGGCACATATTTACCTATGTAACAAACCTGCACATCCTGCATGTATACCCTGGATATACA
TGCCCAGGATATACATTTTATTTAAAATAAAAATAAAAATAATAGATTCATAAAACAGAATATAATTCTGAACTT
TGACTCCCTGTACCTTTAAGAGGGACCCTTAAATTTAAAAATCTATTGTATTTTTTTTTTAGTAGGGGTAGGGAA
TATTTAGGGAATTTGGAAGGGGTTATATAGTTCTTTAAGAATCAAATAGCACATCTTCCTGAAAATAGCACGTAG
ACAAAGTTTTTTTGGAGATAACCTTAGGAATATCGTAACTCTCTGATGCCACCTCCATATGTGATCCTATGTTGA
TTATAAGATTTTGATCAGTGGCTTTCAGACTTTTTTGACTGCAACCTAGAATAAAAGATTCATTTACATTGTGAC
CTAGAACACACACACACACACACACTCTCTCTCCGCCACTCTCCTGCACACAGAAATCATTGATGCTTACAACAA
TTCTTACTCTTACTATGGGTGATTTACTTTGATATGCTCTGTTTTTTTTTTCATTTACAAAACTGTGGATTAATT
TTTTTTGACATGCTAAATTGATCTCAGTAATAGATTGTATTTATTCTTCCTTAGATTCTTCTTTGGAGCAGAATA
AAAGATCTGGCCCATCAGTTCACACAGGTCCAGCGGGACATGTTCACCCTGGAGGACACGCTGCTAGGCTACCTT
GCTGATGACCTCACATGGTGTGGTGAATTCAACACTTCCAGTGAGGCTCTGGGCCCTGTGGGATTGCCCAGGGAT
GTGGAGGGTGAACAGAGTGACTTCTGCTGGAGGCCCTGAATGATTAGTGTGGAGGACAGAGCCACAGGCACCCAT
CCTGATGCCATCTATACTTATATTAGTCCATTTGTGTTGCTATTAAGGAATACCTGAGGCTGCGTAATTTATAAA
GAAAAGAGGTTTATTTGACTCACAGTTACGCAGGCTGTACAAGAAGTAGGGTACCAGCATCCACTTCGGGTGAAG
GCCTGAGGCTGTTTCCACTCATGGAGAAGGGGAAGGGGAGCTGGCATTTACAGAGATCACATGGTGAGGGAGGAA
AGCAAGGAGAGGTCAGGGGAGGTGCCAGGCTGTTTGTAATGACCAGCTGTCCTGGGAACTAGTAGAGTAAGAACT
CATTACTATAAGGACAGCACCATGCCATTCGTGCAGGATCATCCCTATGACCCAAACACCTCCTACTAGTCCCGA
GCTCCAACACTGGGGGTCGAATTTCAACATAAGGTTTGGAGAGTTAAATATCCAAACTATAGCACTACCCTTAAT
GGCAACTCAGGCTGATATAAAGTAGCATTCCCTGTTTTCTTGAAAAATTGACTTCAGAGTTGGGGATTGCCCATG
CTCCCTAATTCCCTTCTTTTGAGTGCTCACATAGCCTGCTTCCGAATTCTTGGTATTTTGCTCTCTGTAAGGTCA
TCATTCAGGTCCAAAGAAGTCTAGAACAGGATGAGGTCTCAGTGGGACCTAGACCAAGGTTCTTGCTCTTCAGAA
TCATCACAGTAGCCATGGACTGGACTCTTCCATCTCAGGCACTGGCTTTGCCATCATTTTTCAGATGTAGCCTTA
TCCTGCCCAGAAAGACTCAACACCTCACCAGGGGAAGGGATTTCCTACAACCAAAACCCTACTGCAGTTTTCACT
TCTTTTTTTTTTCTTTTTGTTTATATGGTGGATATTTTTACTTTATATAGTTTTATTCTTATTTTTACTGTTTTT
CATTGTTTGTTTTTAAAAGCTTATCTTATTATAGCTTCTTTGTCCCAGGTTTGCATTACTTTCAATTACAAAAAT
AAAGCATGATTATTTGAAAAAAAAATACTTGCACATTACAGAAATGCATAAAAGCAAAAAGCAAATGTCACTCTG
AATTTTCCCTTCACCTCCTACCTCCGCATCACTTCTCAAAGGGTAACTATTATCAGCAATTTGATATAGATCTTT
CTAGACTTTTCCTATGCTAATGTAAACATATATATTTAAAATGTACACGCGCTGTTGTGCAACTTGCTTTATTCA
CTTAAAATTGGTAGGTATAAAGATAGCTATCCTCTTTTAAAAGGCTTTATCATTAAGAATCCTATTAATGGATAT
TAAGTTGCTTTAGTTTTGGTTGCTATTATGTCATTATTGTAAGAAACACTTTTGTGCGCACACACACACACACAC
ACACACACACACACACACACACCTGCATACTTGAACGATAAATTTTTATAAATGAAACTTCAATGTTAAAGGATA
AATTGTAATAGAAACTGGTAACATGTCATTTAAAAGATGGTAACTATACCCTCATCAAGAGTATATATATGAGGC
CAGGCACAGTGGCTCATACCTGCAATCCCAGCACTTTGGGAGGTGGAGGAGGGAGGATCACTTGAGCCCAGTAGT
TTAAGAACGGCCTGGGCAACATAGTAAGACCCCATCTCTATTTTACACTAAAAAAAGAAAAAAAAAGAATGTATA
TGAGATAGTTTATTTACCTATATCCCCACTAACACCAGGTATTGTTACTTTAAAATTTTTGGCTCATTTCAGAAG
AAAATAATACCTCAATTTAGTGTGAAGTTCTTTGATGGTGAGGCTACTATATATACATAAATGGTAGATTTTCTG
TTTCTTCTGTAAGCTGGCAGTTCATATATTTTGACCATCTGTATGCGGTATCTATTATTTTCTAATTAGTAGAAG
TTCTTTATAAATTAATAAGAGCTACGATGTATTAAGTACTTACAAAGTGCCAGTGTTCCATGTGCTACGTAAGTA
GTCACTCATTTAATCCTCCACAGCCCCATGAGGTCATATGGTGATCCCATTTTAGAGATAGGAAGTCTGAGGCAT
GGAGTTAAGTAATTTGCCAGCCAGTAAGTGGCAAAGCAAGAAGCAAAGTTTCTCAGACTAACTTGAGAAACTTTG
CTCTTAACTGCCATGTTTTTCTGCCCACTTTCTGGCTCAAGTTGGCGAATATATTTTTCTTATTTTGGACTTTAC
ACGGTGTTTATGGTCTGTCTTTTGCCACCCAGAAATGCAGAAAACCTGTCTGTTCTCTTCCTTATAGCTTCTGTG
TTTCATATCTTCCTTAAAAAGATCTTCTTAGAGAAGCATTCTTCTGTATTTTCACCTACTATTTTTACTTTCACA
ATGTTTAAAATATTTTCCATATTTAGATCTGAGTCTTCCCACCTAGAATATGGTAATATAAATATGTTTTTCCAT
TAATTTTTTTAGATTTTACAGTTTTTCCCATGTTCCATGTTTTCCTTTTTAAATTTCCCTTTTAACAATGACTGT
TTTATTGGTCATTCATTTAACATTTAGCTTTTAAATGTATTTACAGTAGACTTCTCTCCTTTTGTTTTATTTTTT
AATTTGTTCAATTTTTCTTGAAATAAAGTAGAGAAAATGAAATAATTTATTTTTAAGAACTGATTTATTTACAGT
TCAGAGTTCCTTATTTTTGCCTTCTTTTAAATTGAATTATGTATATGTAGTTTTATTTATCTACATTCTAATACT
TTGGCCTCAATTTTTAATTTCTTCTTATTTTATAGATTATCTTTCAAGTTCCTGATGTATATGTATTTATTTACT
TTTTATTCTAAGTTGACAATTTATAATTGTATGTATTTGTGGGGTGAAAAATGAATTTATGAATACAATGTGGGA
TAATTAAATCAAACTAATTAACATATCCACCGCCTCAAATACTTTTTTTAGTTTTTGAGACAGGGTCTCACTCTG
TCACCCAGGCTGAGGTGCAGTGGTGCAATCACAATTCACTGCAACCTTGACCTACCAGGCTCAGGTGGTCCTCCT
ACCTTAGCCTCCCAGGTAGCTGGGACTACAGGTGCCTGCCACCACACTTGGCTAATTTTTTGTATTTTTTTTAGA
GACAGGGTTTCACCATGTTGCCCAGGCTGGTCTTGAACTCCTGGGCTCAAGCGATCTACCCTCTTCAGCCTCCCA
AAGTGTTGGGATTACAGGTGTGAGCCACCAGGCCCGACCTCAAATACTTATTTTTTGTAGTGAGAAAATGTGAAG
TTTACTGTCTTAGCAATGTTGAAATGTACAGCACACTATTATTAACTACAATCACCATGCTGTGCAATAAATATT
TTTAAAAACCCTTTCTAACTGAGATTTTGTACTCTTTGACCATCATCTCCCCATTCCTTCCAACTTCTGGTCTCT
GTATCCACCATTCTATTATCTGCTTCTATGAACTTGATTGTTTTAGATTCCATATGTATTAGGACATGCAGCATT
TGTCTTTCTGTGGGTGGCTTATTTTACTTAGCATATTGTTTTCTTGTTCCATCTATATTGTCACAAATGACAGAA
TTTCTTTCTTTTTAAAGTCTGAATAGTATTCCATTGTGTATATATACCACACTTTATCCATTCGTCTATTGATGG
ACTCAGGTTGATTCCATATCTTGGCTATTGTAAATAGTGCTGCAATGAACATGGGGGAGCAGGTATCTCTTTGAC
AAACTGATTTGAAATCTTTTGGGTAAATACCTAGAAGTGGGATTGCTGGATCATATGGTAGTATTCTATTTTTAG
TTTGTTGAGGAACTTTCATCACATTTTCCATAATGGGTATACTAATTTACTTTCCCAATAGTGTACAAATAACCC
CCTTTCTTCACATTCTTGCCAACACTTGTTATTTATCTTTCATCTTTTTGATTATACCCTTCTGACAGGTGTGAG
ATGATGTCTCATTGTGGTTTTAATTTTTGTTTCCCTATTAATTAGGAAGCTTGAGCATTTTAAAATATATTTGTT
GGCCATTTGTATGTCTTTTGAAAAATGTCTATTCAGGTCCTTTGCCCACCTTTAAATTGATTTTTTTTCTTGTTT
TTGAGTTGTTTGAGTTCCTTATGTATTTTGTTTTTGTTTGTTTTTTAATTTTTAATTTTTGTGCATACATAATAG
GTGTATATATGGGATGTGTGTACATGAGATGTTTTGATATAGATATACAGTGCATAATAATTACATCATGAAAAA
TGTCTCTTTCCCCATAAGCATTTATCTTTTGTGTTACAAACAATCCAATTTTATTCTTTTAGTTATTTTAAAACA
GGGGTGTCCAATTTTTTGGCTTCCCTGGGCCACATTGGAAGAATTGTCTTGGGCCACACATAAAATACACTAATA
CTAATGATAGCTGATGGGCTGAAAAAAAATCGCAAAAAATCTCCTAATTTCTAAGAAAGTTTATGAATTGAACTT
ATGTGTTGGGCTGCATTCAAAGCTGTCATGGGCTGCTTGAGACCCATGGGCCATGGGTTGGACAAGCTTTTTTTA
AAATGTACAACAAAATTGTTATTGACTACAGTCACCATACTGTGCTATCAAATAATAGGTCTTATTCATTCTAAC
TATTTTTTGGTAACCATCCCCACCTCCCCACAATGTCTTGCTACACTTCCCAGCGTCTGGTAACCATTTTTCTAT
TCTCCATGTCCATGAGATCAGTTGTTTTGATTTGTTGGATGCTAAAATAAGTGAGAACATCCTATGTTTATCTTT
CTGTGTCTAGCTTATTTCACTTAACATAATGACCTCCAGTTCTATTCATGTTGTTGCAAATGACAGGAACACATT
CTTTTTTGTGGCTGAATAGTACTCCATTGTGTATAAATACCACATTTTCTTTATCCATTTATCTATTGATGGACA
TTTAGGTTGTTTCCATATCTTGGCTATTGTGAACAGTGCTGCAATAAACATGGGAGTGCAGATATCTCTTCCATT
GACTGATTTTCTTTCTGTTGGGTATATATCCAGCAGTGGCATTGCTGGATCATATAATAGCTCTATTTTTATTTT
TTTGAGAAACCTCAAAACTGTTCTCCATAGTGGTTGTACTAATTCACATTCCCACCAACAGTGTACAAGGGTTCC
CCTTTCTCCACATCCTCATCATTATTTGTTATTGCCTGACTTTTGGATGAAAGCCATTTTAGCTGGGGTGAGATG
ATATCTCATGATAGTTTTGATTTGCATTTATCTGATGGTCAATGATTTGAACACATTTTCATATGCCTGTTTGCC
ATTTGTATGTCTTCTTTTGAGAAATATGTATTCAAATCTTTTGCCCATTTTTAATTGGATTATTAGATTTCTTTC
CTATAGAGTTGTTTGAATTACTTATCTATTCTGGTTTTTAATGCCTTCTTGAATGGGTAGTTTGCAAATATTTTC
TCCCATTCTGTGGGCTCTCTCTTCACTTTGTTGATTGTTTCCTTTGCTATGCAGAAGCTTTTTAACTTGATGTGA
TCCTGTTTGTTCATTTTGCTTTCGTTGCCTGTGCTCATGGGGTATTGCTCAATAATTTTTTTTGCCCAGACAAAT
GTCATGGAGAGTTTCCCCAGTGGTTTCTTGTAGTAGTTTGCAGTAGTTTCATAGTTTGAGGTCTTAGATTTAAAT
CTTTAATCTATTTTGATTTTATTTTTGTATGTGATTTGAGATAGGGGTCTAGTTTCATTTTTATCCATTGAGCCA
CTCTGTGCCTTCTGATTGTAGAGTTTAGTCCATTTACATTTGACGTAAATGTTATATTTTTAAGTAAGGACTTAC
TCCTGCCATTTTGTTACTTGTTTTCTGTTTGTTTTGTGGTCTTCTCTTCCTTCTTTCTTTCCTTTCTGTCTTCCT
TTCAGTGGAGGTGATTTTTTCAGTTTCCTGCTTTTTATTTTTTGTGGAACTGTTATATGTTTTTGAGTTTGAAGT
TACCATGAGGCTTAAAAATAGTATCTTATATCCCATTATTTTAAGCTGATAACAACTTAACACAGTTTGCATAAA
GAAACAAAGACAGCAAACAGAAAGCTAATACAAACTCTATACCTTAACTTCATTCTCCCACTCTAAAACTTTTTG
TTGTTTCTATTTATGTCTTATTGTACTTTATATGTCTTGAAAAGTTATTGTAGTTATTATTTCTGATTGGCTCAT
CATTTAGTTTTTCTACTTAAGACAAGAGTAGTTTACACGTCATAGTTACAGTGTTATAACATTCTGTGGTTTTCT
GTGTACTTACTACTGCCAGTGAGTTTTGTACCTTCAGATGATTAAATTGCTCATTAATATCCTTTTCTTTCTAAT
TGAAGTACTCCCTTTAGCATTTCTTCTAGGACAGGTCTCGTGTTAATTAAATCCCTCACCTTTTGTTTGTCTGGA
AAAGTCTGTGTTTCTCCTTCAAGTTTGAAGGATATTTTCACCAGATATACTATTCTAGAGTAAAAACTTTTTTTT
TTGTCTTTCAGCACTTCAAATATGTCATGCCACTCTCATCTGGCCTGTAAGGTTTCCACTGAAAAGTCTGCTGCC
AGATGTACTGAAACTCCCTTGTATGCTATTTGTTTCTTTTCTCTTGCTGCTTTTAGGATCCTTTCTTTATCTTGG
ACCTTTGGGAGTTTGATTATCAAATGCTTTGGGGCAGCATTCTTGGGTTAAATCTGCTTGGTGTTCTATAACCTT
CTTGTACTTGGGATATTGATATCTTTCTCTAGGTTTGCAAAGTTCTCTGTTATTATTGCTTTGAATAAACTTTCT
ACCTGTATCTCTTTTTCTACCTCCTCTTTGACACCAATAACTCTTGGATTTGCCCTTTTAAGGCAATTTTCTAGA
TCCTGCCAGTGTGCTTCATTGTTTTTTATTCTTTTTTCTTTTGTCTCCTCTGACTGTGTATTTTCAAATAGCCTG
TCTTCAAGCTCACAAATTCTTTCTTCTGCTTGATCAGTTCTGCTATAAAAAGACTCTGATGCATTCTTCAGTGTG
TTATTTGTACTTTTCAGCTCCAGAATTTCTACTTGATTCTTTTTAATTATTTCCATCTCTTTGTTAAATTCATCT
GATAGAATTCTTGAATTTCTTTCAGTTTCCTCAACATGGCTATTTTGAATTCTCTGTCTCACATATCTCTGTTTC
TTCAGGATTGATCTCTGATGTCTTATTTAATTCATTTGGTGAGGTCATGTATTCCTGGATGGTCTTGATACTTGT
AGATATTTTTCTGCATCTAGGCATTGTATTTATTGTAGTCTTTACAACCTGGGCCTGTTTGTACTTGTCCTTGGA
AAGGCTTTCCAGATATTTTGAAGGACTCGGATGTTGTGATCTACGTTGTATCTGCTGTAGGGGGCCCTGCAAGCC
TAGTAATGCTGTGGGTCTTGTACACACTCATGGAGGTACCACCTTGATGGTCTTGGACAAGATCTAGAAGGATTC
TCTGGATTACCAGGCAGAGATTCTTTTTCTAGTCCCTTTACTTTCTCCCAGAGTCTCTCTCTTTCTGTTCTGACC
CACATAAAGCTGGTGACACACTCCACCGCAACTAGGACTTTGCTGGGTAAGACTTGAAGCCAGTACAGCACTTGC
CCAGGGCCTGCAGTAACCACTTCCTAGCTGCCATCTATATTTGCTCAAGGCTCTGGGGCTCTACAATCAGTAGGT
GAGAAAGCCAGCCAGACCCGTGTTCTTCTCTTCAGGTTGGCAAGTTTCCCAAGGCCCTGGGTTGGTCCAGAGGTG
CCATCCAGAAGCCAGGGACTAGAGTAAAAAACCTTAGAAGTCTACCTAGTATTGCATTGTACTGTGACTAAGCTG
GCATTCAAACCACAAGACACAGTCCTTCCCATGCTGTCTTCCCCTTTTCTAAGGCAAAGGAGCCTCACCTCATGG
CCACCACCACCACAGGCCCACAGGGAGTACTGCCAGTGTACTGTTAATATTCCAAGGCCCAAGGACTCTTCAGTC
AGCTTGTGGTTAATGCTGCCTGGCCTGGGACTCACCCTTCAAAGCAGTGGGCTCCCCTCTGGCCCAGGGCAGGCC
CAGAAATGCTATCCAAGAGCCACATCCTGGAATCAGGGACCCCAAGCCCAGTTGGTGCTCTACCTCTCTGTGGCT
GTACCTGAAGCCAGCAAGTCACAGAGTCTCACCCAAGGCCCATGACATACTAATTGGGTATCACTTCTGGTTTTT
CAGGGCCCAAGGGCTCTTCAGTTAGTAGGTGATGAATTCTCTCCAGATGTCGTTGTGAAGATAAAAGAGGTTTAT
TTTCATGAAAACATATATACTTTAAAGCACCTTATGAAATGTATGTCCATTCCACCATCAACATTTTTACCTCTG
TTGGGAAGATAATTCCTTTTGACTCCACAATAATTATTTATATCTACACATGGGAATGTTTCTTTTTTATTTGTG
TGGTTTTGGTTTTAAAGCATTTAATCATTACAAGACTCCTAGAATTACTATATCATGTGCTCTCTGAAGGCAAAG
TTCCCATCTAATTTTTCTATTTTATCTTTCTACCTCTAAGACCTAAAACTCAATAAATGTGCATTAAGGCAGATA
TCCTTGGGAGAAGTGACACAGAAACTATGTATTCATGCTCTGTGTCCATTGTACTTCACTCAGGGTTAAGACTGC
CTTGATGAGGGCAAGTGTAGGAAGACTCTGAGGCCATCTGAGAGTAAGTGGTGAAGACTTAAGAAGTGGGGCAGG
AAACACAGCAAGAGAGAGTTGTCAGGAAGCAGAAAAGCAGTTGGCAAAAGCAACCACTGAAGGACTGGTTTTACC
TCTAATTCTTCCTGGACTGGGGATAATCCTAGAGGGCTTGTCTCTGTCAGATGAACTTTTGGTAGCATTTCCCAG
AACCATGACTCAAAACTTGCCACTGTGTTCCCATCTGGGATTTGGAAGATAAGGTAAGAACTTGGAAAGAATTCA
GGGGACACTTAGTTAAATTGGGTCAAAATGTGTCCATTCTCCAGCCTCCGTCTTGGCAGTGACACATTGGAAAAT
GGTTCCACTATGACTGAACAGCCAGGGAAGAGTACAGCTTATTTATACTCTCTGTTTTCCACTTTATTTTCTACA
AACTATGTCTTTTAGAAATAAACTATCAATCTTGCCCAGACTGGAGAAAGGACTGCAGCAACAACCCTGTTTCAG
TATTCTGGAAAACGGTTTCCCGCAGGGTAAGTACCAAGTAGTGAAATTCTAGAGCTTTGGAGACCACAGAACTTA
AGACGTTACTCAGTCAGTGCTTGGTTTTAACACTTTTGGATTACAAATACTTTTAGGAATGAAAATATAGGATTC
ATTCCTGAGAAAAAGGTTCAGATGCACATGCCAGAAAATTTACACATCCAATTTTAGAACATTCTTAGAGGGTCC
ATGGGCTCCAGTTGCAGAATCTTTGCACGTACCCACTCTGACTTTGGCTACCAGGAACCTGGGGCTTGGTTTAAT
CCTCTGATTCAGGTATTAGTCAATCTTAGATACCTGGGACAGTCGTAACAATCTACATGTATAGACCCCTTACTA
TGTGGCAGGTACGGTCCTCAGATCTTTACATGAACTAGTAACTTGCATCTTCACCAGAACCCTGTGAAGCAGGTG
CCATGAGTATTGTAACCATTTAACACATAACGTGAAGGTACAAGTAAACAAGGAATCTACTAAATGTACAGAATT
AGTAAGAGGCATATGTGGGAGTTTATCCCAAGCTGTCTGACTCCAGATTCAGAATCTAGGCTGGGAAAAACTCAC
CACTCCACCCTCTACCTATTTTTTTTAAAAAAATTGATACATAATAGTTTTACATATTTATGGGGTATATAGTGA
TGTGGTGATACATATAAGGTATAGTGATGAAATCGGGGTAATTAACATATCTGTCATCTCGAACATTTATCATTT
CTTTGTGTTGGGAGCATTGAATATCCCCCTTCTGGCTAGCTGAAACTACATATTATTAACTGTAGTCCTCCTACA
GTGTTATTGAACACCAGAATTTATTCCTCCTATCTAACTATAATTTTGTATCTTTTAACAAATCTCTACCTATCT
CCTCCTCCTCCTACTTTTCTAAGCCTATGGTGGCCTCTGTTCTGCCTTTTACTTCCATAAGATCAACTTAATTTT
AGCTTCCATATATGAGTGAGAGTATGTAGTATTTAACCATCTGTTTCTGGCTTACTTCACTTAACATAATGCCCT
CCAGTTCCTTCCATGTTGCTGCAAATGACAGGAATACTTTTTTTTTTTTTTTTTTTTTTTTTTTTTTTTGAGATG
GAGTCTTGCTCTGTCACCCAGGCTGGAGTGCAATGGTGTGATCTCGGCTCCCTGCAACCACCACCTCCTAAGTTC
AAGCGATTCTCCTACCTCAGCCTCCCCAGTAGCTGGGACTGCAGGTGTGGGCCACTATGCCCGGCTAATTTTTGT
ATTTTCAGTAGAGACGGAGTTTCACCATGTTGGCCAGGCTGGTCTCAAACTCCTGACCTCAGGTGATCCGCCAGC
CTTGGCCTCCCAAGGTGCTAAGACTACAGCCATGGGCCACCATGCCCGGCTAATTTTTGTATTTTCTGTAGAGAC
AGGGTTTCACTATGTTGGCCAGGCTGTCTCAAGCTCCTAACCTCAGGTGATCTACCCACCTTGGCCTCCCAAAGT
TCTGGGATTACAGGGGTGAGCCACTGCACCTGGCCAGGAATACATTTTTTAATTCCTGAATACAATTCCATTGTG
CACATATACCCCCATCTATTTCTAACTTTACTCAAAGCTACCTGTGTATATTTATTTATCTTGTAAGTTGCTTCA
GTGTTAAGTGGACAAGGAAACATTTCTTTTCAAGTGTGTTAGGGAAAAAAAGAGAAAGGAAGGAAGAAAGAAATG
AAAGAAAAAGGTGTGAGTAACAATACACTAATTATAACTTTCAAAATTAAACTTAGACATCTGAGGAACTGGGGC
AGGTGGAAATGTATTTGTTAAGTGCATATGTCTTAGTCCATTTGTGCTGCTATCACAAACTACCTGAGACTTGGT
AATTTTTTTAAAACAGGAGTTTTATTTTCTCATTGTGCTGGATACTTGGGAAATCCAGGATTAAGGTGCCAGCAG
ATTCAATGTCTGGTGAGGGCTGCTGTCTGCTTCCAAAATGGTGCCTTCTTGCTGTGTCCTTACATGGCAGAGGCA
GAGGGGCTAAAGGAACCTAGCTAGTTCCCTGGAGCCCTTTCATAAGGGTGTTAATCCCATTCATGAGGGCAGAAC
CCTTAGGGCCCAATTATCTCCTAAAGGACTCACTACTTATTACCATCACATTGGTCTTAGGTATCAACATAAGAA
ACACATACATTCAAATCATAGCAGCATATCTGTGACAAGCCTTGAAGTAGTTCCTCTGTCATTCCCATTGAGTCA
TCCCCATAGGTAGTGTGACAAATCCCTACATTAAAGGTGAAGAAACTAAGACTCAGAAGTTAAGTGACTCATCCA
AGTTCCCTGGGCTAATAAGTTACAGACCTAAGAGCCTAACCTAGGCCTCCCTGATTCCAAAGCCATGCTCTTCAA
TTTTGTTCTTTGAATCTGCTTATTGGTTCTGTCTTTTAAATGACAGGTTTGATCTTAACTCTAGGTTGGTACCTA
GCTAAATCTCTGTCTTAGGGGGATTCATGTAAACCCTGGTACGATGGAAACAGAAAAACAGCCTGGAAGTTGACA
TAAGGAGACCATGTTTAAACTTGGGCAGATTCCTTTACTCATTCTGATCTTCTGATTCCTCATTTGTCAAATGGA
AATCAAAATATACTTGTTCCATAGGGTTACTGCAATGTTTAAATGAGATACCACCATCCTATAACATAACCCAAA
ATCCACCCACTTCAAAAATAATTCATTGAGTACTTACTATGGACAGTGAACATTCATGGGCACTTTATAGTTTTT
GTTTGTTTGCTTTTTCTGAGAATAGTTTCCATTTCACTACTCTATGGTATGTTTTAGGACAGTGCTGTTGCTAAA
ATCTTTTAAAGCCAAGTCACATTTTATATGTATCAAGAACCTCCTTGCGTTCCCCACTCAGTCCCTGGCACTAGG
AATACAGAGGTGCACGTGATTGAAGGCGTCCTGCCCTTGTGAGCTACCAGCACCTTTATTTTGCCAATCACTCAT
GGATGTATGTGGATGGACTTCTTTTTTCAGACTTGTCCCTTTCTTTTTCTGATAACAGAGGCCATGTTTTTTTTT
AATTTTTAATTTTTGTGGGTAGGTTAAACTCATCATTATAATACAATACAGTTGGATAATGTGGAGGGAATGTAA
GATGCTGTCAGAGTCAGAGAAGGGGACTTGAGCTAGTCCCAGGGGTTGGGGAGGCCTCCTGGAGGGAGCATATAG
AACACTATTTTGTTCATTTCATTTTTCCAAAGTCTAACAAAGATTCCTGCTGAATGTTTCTTGCATGGAGAAATA
AGACCCTTTGCTCAAGCATATTTATTCATTCACTTATTCAGTCCTCCTTTCTCTCTGTGCTTTTCCAGGCCTAAG
GGTCCCCTGTTCTCTCCTCAGGTTCCCCTCTTATGGTGTTCCCATTTCCCTCATCCCTGAATCATCCACCTGTTC
CCACTAAATGAAGCATAATGTTTACAGTGCATGACACTGAGAAAGCACTTTCATCTTCCCCCTCTAGACATTCCT
CTTACTCCTCTGGACTTCTGACTTCTGAACCACTGAACCACCAGCTCTATGAACTATAACACTGAACATTGTTCA
CTTAGAGATTGGAGCAACTGCTTCAAGAACTCTGATATGAAGCATAATCCGTCCAGTGGCTTGGAATAAAAATTG
TGTAGACCTGACATTCCTGGGCTAAAACCATATGGGATATCCTTCCTTAACCAGCTATTGCTAAGTATTGTTTTG
AATGAAACTGCTGGAGGATGGTGATTAAGTTTGCATGATGAATGGTGGGCATTTTTTTTTTTAAGTTTGCAGAAG
CTGCCTGTGATGTGGTCCATGTGATGCTCAATGGATCCCGCAGTAAAATCTTTGACAAAAACAGGTACACATTTA
TTTTGCATCCTGTTTGCAAGTATCCTGTTGCAAATATCACAGTGAATATTTCATCTCTAGAAAGAATATGCTTTT
CATGTTTCAGGTCAGTTCTGAAGATTAGGGCCAAAAAAGGTAAAAATTTTGAATTCCGTGGAGAGAGTTGTCTCC
TGTCAATGTGTTTGTCTGATTTCTCCTTTGCCAAAAATTGTCTACCAGGTTCTAATGGCCACTGCACTGTATCTA
GCCCCTGCTCTTAACTTTTGCAGGCCTGGTGTAATTTTCTCAGCTTTCTCTCCCGTTACCCTCCACCCTACCCAT
TGCTCACCATTGTTCACACCGTTCCCCCATATGACCTGCCTCCCCTGCTCCCCTGCTCCCTTCTGTCTAAATCTT
CACCATCCATGAAGACCTGCCTTGACCCTCCTCTCCTCCAGGAAAATTGTGTACCCCAATTCAGTAGTAAAACTA
CTACCGGGAACATCGGGAACTGTGCTGGGCTCTTGGCCTTCACTATCTTTTTGCAGACATTGTCAACAATGTACT
GTAGTGGTTGAAAGCAGGTACTGGCGGTCATTACATATCATCTGTGTGACCTCAGGCAGGGCAGTCAACCTCTGT
GAGCCCCTGAATATGTACCAAAGAGTTGATGGTGATGGGAAGATTAACTGAGACAACAGATGAAAAATGCTGAGC
TCTGTGCCTGACAACAGAGAAAGTGCTCAATGAGAATCAGCTATTATTCTCATTTGCTGATCCTTGCCACTGAAT
CTGGCCACACCTGTGCCTTCCTTGGCTGATCTCCTTCTATATTTACAGTTTTTACTATGTTGATTACCTTTTCGG
CCTTTGTTCTCTAATTTTTGTTCTCTAATCCCACATAAGGCTGACTGAAAGGAGGAAGCATATATTAATTTGCCT
TATAAACTCTAGGTGCCCCAAATTAATTTTTCTTCTCTCCTGTTTTAATATTTAATTCTACAAGGAAGCATTTGT
CCTTTCGTCTTCTGATCCCAATTTTTTTGGGTAAAAGCATTAACATTTCAGAATTTTATGATCTAATATTATGGT
TCAAGCACTTGAAACAGGAGTGTCAGTTGTCAGAGACTAACAGGGAAGAGTTTAGGAATGGGATTAGGGCAGGCA
ACCATAGTCTTTCAAAGCATTGCCTCTCAAACTTCACTGAGCATGTGAATCACGTGGGGATTGTTCAACTGCAGA
TCATTTCAGCAGGTTATAGTGGTTGAAATTCTACATTTCTTTTTTTTTTTTTTTTTTTTTTTTTTGAGATGGCGT
CTCGCTCTGTTGCCCAGGCTGGAGTGCAGTGACACGGTCTCCGCTCACTGCAAGCTCAGCCTCCCAGGTTCACGC
CATTCTCCTCCCTCAGCCTCCTGAATAACTGGGACTACAGGCACCCACCACCACGCCCAGCTAATTTTTTGTATT
TTTAGTAGAGACGGGGTTTCATAGTGTTAGCCAGGATGGTCTCGATCCCCTGGCATCATGATCTGCCTGCCTCGG
CCTCCCAAATTGCTGGGATTACAGGCATGAGCCACCATGCCTGGCTGAAATTCTACATTTCTAATGAGTTCCCAG
GTGATGTTCATTAGGTTGGTCTAAGGACCACTCTTCAAATAGCAAATATTTAAAGAATCAACATTAATGCACAAA
TTAAGAATTTTATTTTGAGAATCTTGTTAACCGAGGGTCATGCTGAATAAGAAAAGGTTATTGACTGATTTGCAA
TTTGATGTGTCAACTCTAAAGGATAGGTCCTAGCCAGTGCCTTTCTGCCTGCTGGTTGTTGAGGGGGGTGTGGAT
GCTTTCGTTTGGGGTTGATGTTTGGGGTTCTTTGTTTCTTCTATTTTAGCACTTTTGGGAGTGTGGAAGTCCATA
ATTTGCAACCAGAGAAGGTTCAGACACTAGAGGCCTGGGTGATACATGGTGGAAGAGAAGATTCCAGGTATATCT
TACTACTTTGTACCCAAGTGTTATTTTATGAATCAGTCCACAAAAGAATCCACAGTCACAAGCACGCACTGGGAA
CAAATTGACTCAGGAAATGAAACTACATGAATGTGCATGAATCCCAACAGCCTCTTAACTTTATCTCCACAAAGG
ATATTTAACTGCTTGACACTTCAGCTCTCCTGCTGACCCAGGAGCTCTTAGAGGATTTACCTCTACTTTACCTCT
TTATCCAAGGGCCTTGTCCAGGGCGTGCTACAAAAACAAAGAGACTCCAAAAATGTTTGTGAGATCTTGTAATTT
TAATACTTTCTTCTTTCTTCCCCAGAGACTTATGCCAGGATCCCACCATAAAAGAGCTGGAATCGATTATAAGCA
AAAGGAATATTCAATTTTCCTGCAAGAATATCTACAGGTAATTAATTTCTTCTTGAAGAAAAAAATGACTGTCTT
GTCACCTGTAGAATTTCCTTTTTTCCTTAGCCTCCTCTGAGCTTGGAGGGCTGTGTGAATCTTTCTTGGGCCTTG
ATGATGATCACAGATGGCAACCTCTGGTGATCTCTGTCCCTCCTTCCAAGCCGAGTCCAGAAGGTATCCAAGCTA
GTGGCCTTCACTTGGCTGCCTTTCCTCATCCGTCTCTATTGATCCCAAGTAGGACTTGCCTCTAAAGCTGACACA
ACCTTTGATGGCATATTTTTTCATTCCCAGTGTGAGTGGCCCAGTCCAGGGTTCACTGGCCTACTAGGTTTCAGG
GGAGCAAGGGAATGTTTTGCTAAGCCCTTTCTCCCAAGTTGTAAAATCCTTGTGACTTGACATCATTTTGCAAGT
GAAGCTTCCTTAGTTGGATCTGAGTACAGATGCCTAACACATGACAAGGCGTCACACGGCAGTCTACCAAAATCT
ATATTTTTTAAATTAAAAAAAAAAGTATTTACAAAATTTTTCTGATAATTTGTGTTTATTAGAAAACAGTTTAAA
ATTACAGATAGATATATATTTTTTAAAGTCACATATAATTCTAGTTTCAAAACTGAGACCCCTCACTCATTTTTA
AGCAGTTGTGACCAATGGTGTAGGTAGGTACTCATTGGTAGAAGCATCTTTGGAGATTTTTCCACGTATAATAGC
TTGGAACAAGATTGATGCAGAGAGGAAAAGCTGTTCAAAGGAGGTAGAAGCTGAGATGCTAGAATATTGTTCCTG
TTTCCATGTCACTACCTTCTCTCACTAACCACATCAGAAAAGCAGAAGGATAGATTCTGGAGACTCTACTGATGG
CTTTTGTTTCCCAAATGACCTGAATTCCCCATGAGTCACCTTGCTTCTATCTGGAAACAGCCAGAAAAGGCCATG
AGCATTCTACAGCAGTTAGACAGGAAAACAGAAAGAATGAATGAAGGAGCAACTGTAAAAGCAATCTTGCGGCGG
AGGAGCCAAGATGGCCGAATAGGAACAGCTCCGGTCTACAGCTCCCAGCGTGAGCGACGCAGAAGACGGGTGATT
TCTGCATTTCCATCTGAGGTACCGGGTTCATCTCACTAGGGAGTGCCAGACAGTGGGCGCAGGCCAGTGTGTGTG
CACACCGTGCGCGAGCCGAAGCAGGGCGAGGCATTGCCTCACCTGGGAAGCGCAAGGGGTCAGGGAGTTCCCTTT
CCGAGTCAAAGAAAGGGGTGACGGACGCACCTGGAAAATCGGGTCACTCCCACCCGAATATTGCGCTTTTCAGAC
CGGCTTAAGAAACGGCGCACCGCGAGACTATATCCCACACCTGGCTCAGAGGGTCCTACGCCCACGGAATCTCGC
TGATTGCTAGCACAGCAGTCTGAGATCAAACTGCAAGGCGGCAACGAGGCTGGGGGAGGGGCGCCCGCCATTGCC
CAGGCTTGATTAGGTAAACAAAGCAGCCAGGAAGCTCGAACTGGGTGGAGCCCACCACAGCTCAAGGAGGCCTGC
CTGCCTCTGTAGGCTCCACCTCTGGGGGCAGGGCACAGACAAACAAAAAGACAGCAGTAACCTCTGCAGACTTAA
GTGTCCCTGTCTGACAGCTTTGAAGAGAGCAGTGGTTCTCCCAGCACGCAGCTGGAGATCTGAGAACGGGCAGAC
TGCCTCTTCAAGTGGGTCCCTGACCCCTGACCCCCGAGCAGCCTAACTGGGAGGCACCCCCCAGCAGGGGCACAC
TGACACCTCACATGGCAGAGTATTCCAACAGACCTGCAGCTGAGGGTCCTGTCTGTTAGAAGGAAAACTAACAAC
CAGAAAGGACATCTACACCGAAAACCCATCTGTACATCACCATCATCAAAGACCAAAAGTAGATAAAACCACAAA
GATGGGGAAAAAACAGAACAGAAAAACTGGAAACTCTAAAACGCAGAGCGCCTCTCCTCCTCCAAAGGAACGCAG
TTCCTCACCAGCAACAGAACAAAGCTGGATGGAGAATGATTTTGACGAGCTGAGAGAAGAAGGCTTCAGACGATC
AAATTACTCTGAGCTACGGGAGGACATTCAAACCAAAGGCAAAGAAGTTGAAAACTTTGAAAAAAATTTAGAAGA
ATGTATAACTAGAATAACCAATACAGAGAAGTGCTTAAAGGAGCTGATGGAGCTGAAAACCAAGGCTCGAGAACT
ACATGAAGAATGCAGAAGCCTCAGGAGCCGATGCGATCAACTGGAAGAAAGGGTATCAGCAATGGAAGATGAAAT
GAATGAAATGAAGCGAGAAGGGAAGTTTAGAGAAAAAAGAATAAAAAGAAATGAGCAAAGCCTCCAAGAAATATG
GGACTATGTGAAAAGACCAAATCTACGTCTGATTGGTGTACCTGAAAGTGATGTGGAGAATGGAACCAAGTTGGA
AAACACTCTGCAGGATATTATCCAGGAGAACTTCCCCAATCTAGCAAGGCAGGCCAACGTTCAGATTCAGGAAAT
ACAGAGAACGCCACAAAGATACTCCTCGAGAAGAGCAACTCCAAGACACATAATTGTCAGATTCACCAAAGTTGA
AATGAAGGAAAAAATGTTAAGGGCAGCCAGAGAGAAAGGTCAGGTTACCCTCAAAGGAAAGCCCATCAGACTAAC
AGCGGATCTCTCGGCAGAAACCCTACAAGCCAGAAGAGAGTGGGGGCCAATATTCAACATTCTTAAAGAAAAGAA
TTTTCAACCCAAAATTTCATATCCAGCCAAACTAAGCTTCATAAGTGAAGGAGAAATAAAATACTTTATAGACAA
GCAAATGCTGAGAGATTTTGTCACCACCAGGCCTGCCCTAAAAGAGCTCCTGAAGGAAGAGCTAAACATGGAAAG
GAACAACCGGTACCAGCCGCTGCAAAATCATGCCAAAATGTAAAGACCATCGAGACTAGGAAGAAACTGCATCAA
CTAATGAGCAAAATCACCAGCTAACATCATAATGACAGGATCAAATTCACACATAACAATATTAACTTTAAATAT
AAATGGACTAAATTCTGCAATTAAAAGACACAGACTGGCAAGTTGGATAAAGAGTCAAGACCCATCAGTGTGCTG
TATTCAGGAAACCCATCTCACGTGCAGAGACACACATAGGCTCAAAATAAAAGGATGGAGGAAGATCTACCAAGC
CAATGGAAAACAAAAAAAGGCAGGGGTTGCAATCCTAGTCTCTGATAAAACAGACTTTAAACCAACAAAGATCAA
AAGAGACAAAGAAGGCCATTACATAATGGTAAAGGGATCAATTCAACAAGAGGAGCTAACTATCCTAAATATTTA
TGCACCCAATACAGGAGCACCCAGATTCATAAAGCAAGTCCTCAGTGACCTACAAAGAGACTTAGACTCCCACAC
ATTAATAATGGGAGACTTTAACACCCCACTGTCAACATTAGACAGATCAACGAGACAGAAAGTCAACAAGGATAC
CCAGGAATTGAACTCAGCTCTGCACCAAGCAGACCTAATAGACATCTACAGAACTCTCCACCCCAAATCAACAGA
ATATACATTTTTTTCAGCACCACACCACACCTATTCCAAAATTGACCACATAGTTGGAAGTAAAGCTCTCCTCAG
CAAATGTAAAAGAACAGAAATTATAACAAACTATCTCTCAGACCACAGTGCAATCAAACTAGAACTCAGGATTAA
GAATCTCACTCAAAGCCGCTCAACTACATGGAAACTGAACAACCTGCTCCTGAATGACTACTGGGTACATAACGA
AATGAAGGCAGAAATAAAGATGTTCTTTGAAACCAACGAGAACAAAGACACCACATACCAGAATCTCTGGGACGC
ATTCAAAGCAGTGTGTAGAGGGAAATTTATAGCACTAAATGCCTACAAGAGAAAGCAGGAAAGATCCAAAATTGA
CACCCTAACATCACAATTAAAAGAACTAGAAAAGCAAGAGCAAACACATTCAAAAGCTAGCAGAAGGCAAGAAAT
AACTAAAATCAGAGCAGAACTGAAGGAAATAGAGACACAAAAAACCCTTCAAAAAATCAATGAATCCAGGAGCTG
GTTTTTTGAAAGGATCAACAAAATTGATAGACCGCTAGCAAGACTAATAAAGAAAAAAAGAGAGATGAATCAAAT
AGACACAATAAAAAATGATAAAGGGGATATCACCACCGATCCCACAGAAATACAAACTACCATCAGAGAATACTA
CAAACACCTCTACGCAAATAAACTAGAAAATCTAGAAGAAATGGATACATTCCTCGACACATACACTCTCCCAAG
ACTAAACCAGGAAGAAGTTGAATCTCTGAATAGACCAATAACAGGCTCTGAAATTGTGGCAATAATCAATAGTTT
ACCAACCAAAAAGAGTCCAGGACCAGATGGATTCACAGCCGAATTCTACCAGAGGTACAAGGAGGAACTGGTACC
ATTCCTTCTGAAACTATTCCAATCAATAGAAAAAGAGGGAATCCTCCCTAACTCATTTTATGAGGCCAGCATCAT
TCTGATACCAAAGCCGGGCAGAGACACAACCAAAAAAGAGAATTTTAGACCAATATCCTTGATGAACATTGATGC
AAAAATCCTCAATAAAATACTGGCAAACCGAATCCAGCAGCACATCAAAAAGCTTATCCACCATGATCAAGTGGG
CTTCATCCCTGGGATGCAAGGCTGGTTCAATATACGCAAATCAATAAATGTAATCCAGCATATAAACAGAGCCAA
AGACAAAAACCACATGATTATCTCAATAGATGCAGAAAAAGCCTTTGACAAAATTCAACAACGCTTCATGCTAAA
AACTCTCAATAAATTAGGTATTGATGGGACGTATTTCAAAATAATAAGAGCTATCTATGACAAACCCACAGCCAA
TATCATACTGAATGGGCAAAAACTGGAAGCATTCCCTTTGAAAACTGGCACAAGACAGGGATGCCCTCTCTCACC
GCTCCTATTCAACATAGTGTTGGAAGTTCTGGCCAGGGCAATCAGGCAGGAGAAGGAAATAAAGGGTATTCAATT
AGGAAAAGAGGAAGTCAAATTGTCCCTGTTTGCAGACGACATGATTGTTTATCTAGAAAACCCCATCGTCTCAGC
CCAAAATCTCCTTAAGCTGATAAGCAACTTCAGCAAAGTCTCAGGATACAAAATCAATGTACAAAAATCACAAGC
ATTCTTATACACCAACAACAGACAAACAGAGAGCCAAATCATGGGTGAACTCCCATTCACAATTGCTTCAAAGAG
AATAAAATACCTAGGAATCCAACTTACAAGGGATGTGAAGGACCTCTTCAAGGAGAACTACAAACCACTGCTCAA
GGAAATAAAAGAGGACACAAACAAATGGAAGAACTGCTCATGGGTAGGAAGAATCAATATCGTGAAAATGGCCAT
ACTGCCCAAGGTAATTTACAGATTCAATGCCATCCCCATCAAGCTACCAATGACTTTCTTCACAGAATTGGAAAA
AACTACTTTAAAGTTCATATGGAACCAAAAAAGAGCCCGCATTGCCAAGTCAATCCTAAGCCAAAAGAACAAAGC
TGGAGGCATCACACTACCTGACTTCAAACTATACTACAAGGCTCCAGTAACCAAAACAGCATGGTACTGGTACCA
AAACAGAGATATAGATCAATGGAACAGAACAGAGCCCTCAGAAATAATGCCGCATATCTACAACTATCTGATCTT
TGACAAACCTGAGAAAAACAAGCAATGGGGAAAGGATTCCCTATTTAATAAATGGTGCTGGGAAAACTGGCTAGC
CATATGTAGAAAGCTGAAACTGGATCCCTTCCTTACACCTTATACAAAAATCAATTCAAGATGGATTAAAGATTT
AAACGTTAAACCTAAAACCATAAAAACCCTAGAAGAAAACCTAGGCATTACCATTCAGGACATAGGCGTGGGCAA
GGACTTCATGTCCAAAACACCAAAAGCAATGGCAACAAAAGACAAAATTGACAAATGGGATCTAATTAAACTAAA
GAGCTTCTGCACAGCAAAAGAAACTACCATCAGAGTGAACAGGCAACCTACAACATGGGAGAAAATTTTCGCAAC
CTACTCATCTGACAAAGGGCTAATATCCAGAATCTACAATGAACTCAAACAAATTTACAAGAAAAAAACAAACAA
CCCCATCAAAAAGTGGGCGAAGGACATGAACAGACACTTCTCAAAAGAAGACATTTATGCAGCCAAAAAACACAT
GAAGAAATGCTCATCATCACTGGCCATCAGAGAAATGCAAATCAAAACCACTATGAGATATCATCTCACACCAGT
TAGAATGGCAATCATTAAAAAGTCAGGAAACAACAGGTGCTGGAGAGGATGCGGAGAAATAGGAACACTTTTACA
CTGTTGGTGGGACTGTAAACTAGTTCAACCATTGTGGAAGTCAGTGTGGCGATTCCTCAGGGATCTAGAACTAGA
AATACCATTTGACCCAGCCATCCCATTACTGGGTATATAACCAAATGAGTATAAATCATGCTGCTATAAAGACAC
ATGCACACGTATGTTTATTGCGGCACTATTCACAATAGCAAAGACTTGGAACCAACCCAAATGTCCAACAATGAT
AGACTGGATTAAGAAAATGTGGCACATATACACCATGGAATACTATGCAGCCATAAAAAATGATGAGTTCATATC
CTTTGTAGGGACATGGATGAAATTGGAAACCATCATTCTCAGTAAACTATCGCAAGAACAAAAAACCAAACACCG
CATATTCTCACTCATAGGTGGGAATTGAACAATGAGATCACATGGACACAGGAAGGGGAATATCACACTCTGGGG
ACTGTGGTGGGGTCGGGGGAGGGGGGAGGGATAGCATTGGGAGATATACCTAATGCTAGATGACACATTAGTGGG
TGCAGCGCACCAGCATGGCACATGTATACATATGTAACTAACCTGCACAATGTGCACATGTACCCTAAAACTTAG
AGTATAATAAAAAAAATAAAAAATAAAAAACAACTCTCAGAAGCAAAAAAAAAAAAAAAAAAAAAAAAAAAAGCA
ATCTTGCAGATATCTGACGAGTCTAAGCTGTTCAAAGATATGTTGCATGGAGAAAATAGAATAGTAGAAACCTAG
ACAAAGACTGGGAAATAAAGATGGTCTTATCCCCAATACTCTTTTACCTTTTTTGTCTTATGAAACATTAACCTT
TTTCTCATAAATGACCAGAAGACCTTTATATTATAATTCGTCAACTCCCCTCATTTGTGTCTGCTTTAGGCTCCA
AGTGAGCTCACTCATTCTCCATCTGGAAAGAAAATATGGGCATGGCTTCCATTTGGACTTGTACAGACAGTGGCC
CATAATGGGAACCAGGTGACACATCACAAGGGCAGGTTCTGACACCTCTTCCTTCCAGAAGCCCAGGGGTGCTGG
CAGCTGCTTCTGAGGATCTCTCTCTTCCTTGGCTCATATTTAGCAAAATCAAATTTAAAGAACCCCATTCCTCGC
TATCCACCATCCCCCTATTCATGTGCCAGCCACTCCTATTGGATCCTGTTGCTTTAGCTAATTTTTATGAAAATA
ATAGTCATTCACCTGTTAGGTACTTATCTAAGGTTTGTTTCAAAGCAAGTTTGGTCCCCTTGCTGAGGGTCTCCA
GCTTTTTCCCAGACTCTGCCTCTGACCCTGGATTCAACATTCCCTCAGGAAGCTTCGGAAGAGAGGAAAGCAAAT
TAGCCACAGAAGCTGTGGGGGTCCGTGGCCTTGGTTGCTGCTCCTGCTGTTTTTTTGACCAGCAGGTGGCATGGA
TAGCTCCCCTCCCGACATGTCACTGCAGGAGAGGAGTTTATATGGATGCTAAGTGGTCTGTGCACCTTGTCGTCG
CTAAAAAAGGGGCTTCCTCCATTAGCGAATTGGACGACAGATGTATCCTACGGTCTCTTGATTTCCTTTTTTGCT
TTCTTGTCATAGACCTGACAAGTTTCTTCAGTGTGTGAAAAATCCTGAGGATTCATCTTGCACATCTGAGATCTG
AGCCAGTCGCTGTGGTTGTTTTAGCTCCTTGACTCCTTGTGGTTTATGTCATCATACATGACTCAGCATACCTGC
TGGTGCAGAGCTGAAGATTTTGGAGGGTCCTCCACAATAAGGTCAATGCCAGAGACGGAAGCCTTTTTCCCCAAA
GTCTTAAAATAACTTATATCATCAGCATACCTTTATTGTGATCTATCAATAGTCAAGAAAAATTATTGTATAAGA
TTAGAATGAAAATTGTATGTTAAGTTACTTCACTTTAATTCTCATGTGATCCTTTTATGTTATTTATATATTGGT
AACATCCTTTCTATTGAAAAATCACCACACCAAACCTCTCTTATTAGAACAGGCAAGTGAAGAAAAGTGAATGCT
CAAGTTTTTCAGAAAGCATTACATTTCCAAATGAATGACCTTGTTGCATGATGTATTTTTGTACCCTTCCTACAG
ATAGTCAAACCATAAACTTCATGGTCATGGGTCATGTTGGTGAAAATTATTCTGTAGGATATAAGCTACCCACGT
ACTTGGTGCTTTACCCCAACCCTTCCAACAGTGCTGTGAGGTTGGTATTATTTCATTTTTTAGATGAGAAAATGG
GAGCTCAGAGAGGTTATATATTTAAGTTGGTGCAAAAGTAATTGCAAGTTTTGCCACCGAAAGGAATGGCAAAAC
CACAATTATTTTTGAACCAACCTAATAATTTACCGTAAGTCCTACATTTAGTATCAAGCTAGAGACTGAATTTGA
ACTCAACTCTGTCCAACTCCAAAATTCATGTGCTTTTTCCTTCTAGGCCTTTCATACCAAACTAATAGTAGTTTA
TATTCTCTTCCAACAAATGCATATTGGATTAAATTGACTAGAATGGAATCTGGAATATAGTTCTTCTGGATGGCT
CCAAAACACATGTTTTTCTTCCCCCGTCTTCCTCCTCCTCTTCATGCTCAGTGTTTTATATATGTAGTATACAGT
TAAAATATACTTGTTGCTGGTACTGGCAGCTTATATTTTCTCTCTTTTTTCATGGATTAACCTTGCTTGAGGGCT
TTAACAATTGTATTACTTTTTCAAAGAACTAAGCTTTAGCTTCATTGATTTTTTTCTATTTAATTGGGTTTTGCT
CTTCTCTTTAGCATTGGAAACATAGAAATGCTTTCTGATTTCTTTGGGTAGATTTACGTATTCAGCTTCTTGAGA
TGGAAGTTTAGATCACTGATCCTTCAGCTTGTTTTCTTTTTTGTATACATAGATTTTAGGACGATATATTTTCCC
TTGAGTTCTGCTTTAGCTGCAGCTCTTATGTTTTGATATGCCTCTCTTTATTATCCTTCAGTTAAAAATATCTTT
CAATTCATTGTTATATAAAAATATGTGCCTAGTTTTTAACATCTGGAGATTTTCTAGTTTTGAAAAAAACATAAG
CCAGGCATGGTGGCTCACACCTGTATCCCCAGCACTTTGGGAGGCCGAGACGGGAGGATCGCCTGAGCTCAGGAG
TTTTTACACCAGCCTGGGAATAACAGTGAGACATTATCTCCAAAAAAATTACCTGGGTATGGTGTTGTGCACCTG
TAGTCCCAGCTACTCTGGAGACTGAGGTGGGAGGATTGTTTGAGCTTGGGAGGTTGAGGCTGCAGGGAGCTGTGA
TCACACCACTGCACTCTGGCCTGAGTGACAGATTGAGACCCTGTCTCAATAAAAGCAAAAATAAAGAAAATAAAC
CATATGTGTTGAACAAAGGATTAATAAATTAATTTGAGACTCCTTCAGGGAATGACCACAATTTATTGAAAATAG
CCTAAATGTTGGAGTCAGGCATTTCTGGATTCATATTTTGACATCATGCTGTCATCTTGAACAAAATGCCTAACC
TTTCTGAACTTCAACTTCCTTGCCACTCAAATAAGGATTACAAAACTTAAAATGTGGTAAGTACTAAAGACGACA
GCAAAAATTGAGTCCAGCACAGAGCTTCCTAAATAAGCAAGCACTCAACAGAGTTGGTTCCTTTCTTCCTCCCCT
GCTTGACAATCCAGTTTCCCACAGGAGCCTTTGTAGCTGTAGCCACCATGGTCAGTCCAGGGATTCTTCACTAGC
CCCTTCTCCCCTGGCAGACATCCTTGTGGGAGTTTAGTCTTGGCTCGACATGAGGATGGGGGTTTGGGACCAGTT
CTGAGTGAGAATCAGACTTGCCCCAAGTTGCCATTAGCTCCCCCTGCAGAATGTCTTCAGAATCGGGGCCCGGTC
AGTCTCCTGGGTGACCTGCTGTTTTCCTCTTAAGATCCTTTCCACTTTGGTTGCTGCTTTCGGGACTCATCGAGT
CCTTGCTCAACAGGATACCCCTTGAAGTGGCTGCCTGGGCCACATCCCCTTCCAAACAAGAAATCAAAATATTAG
AAATCAATTTTTGAAATTTCCCCTAGGAAGACTCATTTGAGTGTTCAAGTTCAGAGCCAGTGGAGACCTTAGGGG
AGGGTGGTCACAAGGATTTTGCACAGTGCTTTAGAGGGTCCCAGGGAGCCACAGAGGTGGTGAGGGGCTGGGTGC
TCTTTTCTCCGTGCATGACCTTGTGTGTCTATCTTCATTACCACAATGCCTCATCTCTACCTCCTTTCCCCCTGT
AGTTCCAACGTGGGTATCTTTGCCATCTCTGGCCCGAAGGACTTTCTGACCTACATGTATAAATACCCCCTCACA
ATATATATTACTTTTCCTATAAGTGACTTCTCTACTGGATTACTGGTTGCTCATACACCTCATATTTTACTCGTA
AATCTACTACTCCCTGTCTGCCTACTCCATTCTCATTTGCTGTAGAAAATTCTCTTACCATCCCAACTTTCACCC
ACCATCATGCTTACCCAAAGGCTGTGGGAATGACCTGGGCCCTAATGCCCCTTTTCTAAATTCCTAAGGCTCACC
ATTTTCCTATTGTAATGGTTCTTGACCTTATAATGTTTGAGGCACCTTTTCAAATATAGTCCTTTGATTTCAGAC
TGAATACTTGAAAGGACACACACACACATACGTAAGTGCATATGACTGCATACACCCACACACACACACGTGCCT
GTATACAGTCATATGATACATACACAAACACACGCACACAAGCCTGCATACATCATATGCCAACAGTGGGGATAT
GTTCTGAGAAATGCATCATTAGATGATTTTGTCATTGTGTGAACATCATAGAGTGTACTTACACTAACCTAGATG
GTCTAACCTACTACACACCCAGGCTACATGGTATCACCTATTCCTCCTAGGCTACAAGCCTGTACAGCGTGTGTC
TGTACTAAATGCTGTGGGCAATTTTAACCTGATGGTAAATGTTTGTGTATCTAAACATATCTAAACATAGAAAAG
GTACAGTAAACATGCAGTATTATAATCTTATGAGACCGTCATCATATATGTGGTCCACTGTTTGGGCCATCATTG
GCTGAAAAGTGGTTATGCGACACATGACTGTATATATACTTTCCTGTTACAACAACAGTGTCTCTCAATCCACAG
TAATTGCAGCATCCAGTAGGTCTTACTTTAGCCCTGAGTCACCATTTGTGTCAACGTGTTTAGTGCCATGTCCAC
GTCTCTCATGTAACTGGCAGAGCTATCAAATATTTTGGCAAAACACATTGTTTCTTTGGCTTTGCCTTGGTAACT
TTCTGTGCCTTTTGTAGCTCTTGTTTGGAAGAAGCTCAACCCATGTCTGCACACTGTGATACAAGGGGGACAGCA
TCGACATCGACTTACTTCTTGGTGCCTTATTCCTCCTTAGAACAATTCCTAAATCTGTAACTTAAGTTTCTCAGG
AAGATTCCATACTGCACAGAAAACTGCTTTTGTGGGTTTTTAAAAGGCAAGTTGTTATATGTGCTGGATAGTTTT
TAAGTATGACATAAAAATTGTATAAAGTAAAATATTAAAATACACCTAGAATACTGTATAACTTTAAGTCATTTT
ATCAACACATTGCTAATCCAGATATTTTCCCGCAGTTTTTCTTTGAATAACAGAGCAATTAATTTACTTTTACTA
TGAAGAGTCATCATTTTAGTATGTATTTTAAGCAATCCACCAAGAACTCAGTAGGCAGCTGAGAGGTGCTGCCCA
GAGAAGTGGTGATTAGCTTGGCCTTAGCTCACCCACACAAAGCACAACAGGCTTTGAACTATTCCCTAACGGGGC
ATTTATTCTTTTTTTTTTTTTTTTTTGGGAGACGGAGTCTCGCTGTCGCCCAGGCTAGAGTGCAGTGGCGCGATC
TCGGCTCACTGCAGGCTCCACCCCCTGGGGTTCACGCCATTCTCCTGCCTCAGCCTCCCAAGTAGCTGGGACTGC
AGGCGCCCGCCATCTCGCCCGGCTAATTTTTTGTATTTTTAGTAGAGACGGGGTTTCACCGTGTTAGCCAGGATA
GGGCATTTATTCTTGAACTTGATTCAGAGAGGCACACATTACCATTCTCTAATCAGAATGCAAGTAGCGCAAGGC
GGTGGAAACTATGGAATTCGGAGGCAGGTGATGCATTGGGCGAGTTTATTAACATCTGTGACTCTCTAGTTTGAA
ATTTATTTGTAACAGACAAAAATGAATTAAACAAACAATAAAAGTATAATAAAGAA

A representative mRNA sequence of CD38 (transcript variant 1) is provided by NCBI Reference Sequence No: NM_001775.4, shown below:

| (SEQ ID NO: 62) | |
|---|---|
| 1 | gcagtttcag aacccagcca gcctctctct tgctgcctag cctcctgccg gcctcatctt |
| 61 | cgcccagcca accccgcctg gagccctatg gccaactgcg agttcagccc ggtgtccggg |
| 121 | gacaaaccct gctgccggct ctctaggaga gcccaactct gtcttggcgt cagtatcctg |
| 181 | gtcctgatcc tcgtcgtggt gctcgcggtg gtcgtcccga ggtggcgcca gcagtggagc |
| 241 | ggtccgggca ccaccaagcg ctttcccgag accgtcctgg cgcgatgcgt caagtacact |
| 301 | gaaattcatc ctgagatgag acatgtagac tgccaaagtg tatgggatgc tttcaagggt |
| 361 | gcatttattt caaaacatcc ttgcaacatt actgaagaag actatcagcc actaatgaag |
| 421 | ttgggaactc agaccgtacc ttgcaacaag attcttcttt ggagcagaat aaaagatctg |
| 481 | gcccatcagt tcacacaggt ccagcgggac atgttcaccc tggaggacac gctgctaggc |
| 541 | taccttgctg atgacctcac atggtgtggt gaattcaaca cttccaaaat aaactatcaa |
| 601 | tcttgcccag actggagaaa ggactgcagc aacaaccctg tttcagtatt ctggaaaacg |
| 661 | gtttcccgca ggtttgcaga agctgcctgt gatgtggtcc atgtgatgct caatggatcc |
| 721 | cgcagtaaaa tctttgacaa aaacagcact tttgggagtg tggaagtcca taatttgcaa |
| 781 | ccagagaagg ttcagacact agaggcctgg gtgatacatg gtggaagaga agattccaga |
| 841 | gacttatgcc aggatcccac cataaaagag ctggaatcga ttataagcaa aaggaatatt |
| 901 | caattttcct gcaagaatat ctacagacct gacaagtttc ttcagtgtgt gaaaaatcct |
| 961 | gaggattcat cttgcacatc tgagatctga gccagtcgct gtggttgttt tagctccttg |
| 1021 | actccttgtg gtttatgtca tcatacatga ctcagcatac ctgctggtgc agagctgaag |
| 1081 | attttggagg gtcctccaca ataaggtcaa tgccagagac ggaagccttt ttccccaaag |
| 1141 | tcttaaaata acttatatca tcagcatacc tttattgtga tctatcaata gtcaagaaaa |
| 1201 | attattgtat aagattagaa tgaaaattgt atgttaagtt acttcacttt aattctcatg |
| 1261 | tgatcctttt atgttattta tatattggta acatcctttc tattgaaaaa tcaccacacc |
| 1321 | aaacctctct tattagaaca ggcaagtgaa gaaaagtgaa tgctcaagtt tttcagaaag |
| 1381 | cattacattt ccaaatgaat gaccttgttg catgatgtat ttttgtaccc ttcctacaga |
| 1441 | tagtcaaacc ataaacttca tggtcatggg tcatgttggt gaaaattatt ctgtaggata |
| 1501 | taagctaccc acgtacttgg tgctttaccc caacccttcc aacagtgctg tgaggttggt |
| 1561 | attatttcat tttttagatg agaaaatggg agctcagaga ggttatatat ttaagttggt |
| 1621 | gcaaaagtaa ttgcaagttt tgccaccgaa aggaatggca aaaccacaat tatttttgaa |
| 1681 | ccaacctaat aatttaccgt aagtcctaca tttagtatca agctagagac tgaatttgaa |
| 1741 | ctcaactctg tccaactcca aaattcatgt gctttttcct tctaggcctt tcataccaaa |
| 1801 | ctaatagtag tttatattct cttccaacaa atgcatattg gattaaattg actagaatgg |
| 1861 | aatctggaat atagttcttc tggatggctc caaaacacat gtttttcttc ccccgtcttc |
| 1921 | ctcctcctct tcatgctcag tgttttatat atgtagtata cagttaaaat atacttgttg |
| 1981 | ctggtactgg cagcttatat tttctctctt ttttcatgga ttaaccttgc ttgagggctt |
| 2041 | taacaattgt attacttttt caaagaacta agctttagct tcattgattt ttttctattt |
| 2101 | aattgggttt tgctcttctc tttagcattg gaaacataga aatgctttct gatttctttg |
| 2161 | ggtagattta cgtattcagc ttcttgagat ggaagtttag atcactgatc cttcagcttg |
| 2221 | ttttcttttt tgtatacata gattttagga cgatatattt tcccttgagt tctgctttag |
| 2281 | ctgcagctct tatgttttga tatgcctctc tttattatcc ttcagttaaa aatatctttc |
| 2341 | aattcattgt tatataaaaa tatgtgccta gtttttaaca tctggagatt ttctagtttt |
| 2401 | gaaaaaaaca taagccaggc atggtggctc acacctgtat ccccagcact ttgggaggcc |
| 2461 | gagacgggag gatcgcctga gctcaggagt ttttacacca gcctgggaat aacagtgaga |
| 2521 | cattatctcc aaaaaaatta cctgggtatg gtgttgtgca cctgtagtcc cagctactct |
| 2581 | ggagactgag gtgggaggat tgtttgagct tgggaggttg aggctgcagg gagctgtgat |
| 2641 | cacaccactg cactctggcc tgagtgacag attgagaccc tgtctcaata aaagcaaaaa |
| 2701 | taaagaaaat aaaccatatg tgttgaacaa aggattaata aattaatttg agactccttc |
| 2761 | agggaatgac cacaatttat tgaaaatagc ctaaatgttg gagtcaggca tttctggatt |
| 2821 | catattttga catcatgctg tcatcttgaa caaaatgcct aacctttctg aacttcaact |
| 2881 | tccttgccac tcaaataagg attacaaaac ttaaaatgtg gtaagtacta aagacgacag |
| 2941 | caaaaattga gtccagcaca gagcttccta aataagcaag cactcaacag agttggttcc |
| 3001 | tttcttcctc ccctgcttga caatccagtt tcccacagga gcctttgtag ctgtagccac |
| 3061 | catggtcagt ccagggattc ttcactagcc ccttctcccc tggcagacat ccttgtggga |
| 3121 | gtttagtctt ggctcgacat gaggatgggg gtttgggacc agttctgagt gagaatcaga |
| 3181 | cttgccccaa gttgccatta gctccccctg cagaatgtct tcagaatcgg ggcccggtca |
| 3241 | gtctcctggg tgacctgctg ttttcctctt aagatccttt ccactttggt tgctgctttc |
| 3301 | gggactcatc gagtccttgc tcaacaggat accccttgaa gtggctgcct gggccacatc |
| 3361 | cccttccaaa caagaaatca aaatattaga aatcaatttt tgaaatttcc cctaggaaga |
| 3421 | ctcatttgag tgttcaagtt cagagccagt ggagacctta ggggagggtg gtcacaagga |
| 3481 | ttttgcacag tgctttagag ggtcccaggg agccacagag gtggtgaggg gctgggtgct |
| 3541 | cttttctccg tgcatgacct tgtgtgtcta tcttcattac cacaatgcct catctctacc |
| 3601 | tcctttcccc ctgtagttcc aacgtgggta tctttgccat ctctggcccg aaggactttc |
| 3661 | tgacctacat gtataaatac cccctcacaa tatatattac ttttcctata agtgacttct |
| 3721 | ctactggatt actggttgct catacacctc atattttact cgtaaatcta ctactccctg |
| 3781 | tctgcctact ccattctcat ttgctgtaga aaattctctt accatcccaa ctttcaccca |
| 3841 | ccatcatgct tacccaaagg ctgtgggaat gacctgggcc ctaatgcccc ttttctaaat |
| 3901 | tcctaaggct caccattttc ctattgtaat ggttcttgac cttataatgt ttgaggcacc |
| 3961 | ttttcaaata tagtcctttg atttcagact gaatacttga aaggacacac acacacatac |
| 4021 | gtaagtgcat atgactgcat acacccacac acacacacgt gcctgtatac agtcatatga |
| 4081 | tacatacaca aacacacgca cacaagcctg catacatcat atgccaacag tggggatatg |
| 4141 | ttctgagaaa tgcatcatta gatgattttg tcattgtgtg aacatcatag agtgtactta |
| 4201 | cactaaccta gatggtctaa cctactacac acccaggcta catggtatca cctattcctc |
| 4261 | ctaggctaca agcctgtaca gcgtgtgtct gtactaaatg ctgtgggcaa ttttaacctg |
| 4321 | atggtaaatg tttgtgtatc taaacatatc taaacataga aaaggtacag taaacatgca |
| 4381 | gtattataat cttatgagac cgtcatcata tatgtggtcc actgtttggg ccatcattgg |
| 4441 | ctgaaaagtg gttatgcgac acatgactgt atatatactt tcctgttaca acaacagtgt |
| 4501 | ctctcaatcc acagtaattg cagcatccag taggtcttac tttagccctg agtcaccatt |
| 4561 | tgtgtcaacg tgtttagtgc catgtccacg tctctcatgt aactggcaga gctatcaaat |
| 4621 | attttggcaa aacacattgt ttctttggct ttgccttggt aactttctgt gccttttgta |
| 4681 | gctcttgttt ggaagaagct caacccatgt ctgcacactg tgatacaagg gggacagcat |
| 4741 | cgacatcgac ttacttcttg gtgccttatt cctccttaga acaattccta aatctgtaac |
| 4801 | ttaagtttct caggaagatt ccatactgca cagaaaactg cttttgtggg tttttaaaag |
| 4861 | gcaagttgtt atatgtgctg gatagttttt aagtatgaca taaaaattgt ataaagtaaa |
| 4921 | atattaaaat acacctagaa tactgtataa ctttaagtca ttttatcaac acattgctaa |
| 4981 | tccagatatt ttcccgcagt ttttctttga ataacagagc aattaattta cttttactat |
| 5041 | gaagagtcat cattttagta tgtattttaa gcaatccacc aagaactcag taggcagctg |
| 5101 | agaggtgctg cccagagaag tggtgattag cttggcctta gctcacccac acaaagcaca |
| 5161 | acaggctttg aactattccc taacggggca tttattcttt tttttttttt tttttgggag |
| 5221 | acggagtctc gctgtcgccc aggctagagt gcagtggcgc gatctcggct cactgcaggc |
| 5281 | tccaccccct ggggttcacg ccattctcct gcctcagcct cccaagtagc tgggactgca |
| 5341 | ggcgcccgcc atctcgcccg gctaattttt tgtattttta gtagagacgg ggtttcaccg |
| 5401 | tgttagccag gatagggcat ttattcttga acttgattca gagaggcaca cattaccatt |
| 5461 | ctctaatcag aatgcaagta gcgcaaggcg gtggaaacta tggaattcgg aggcaggtga |
| 5521 | tgcattgggc gagtttatta acatctgtga ctctctagtt tgaaatttat ttgtaacaga |
| 5581 | caaaaatgaa ttaaacaaac aataaaagta taataaagaa |

A representative amino acid sequence of CD38 is provided by NCBI Reference Sequence No. NP 001766.2, shown below:

(SEQ ID NO: 63)
MANCEFSPVSGDKPCCRLSRRAQLCLGVSILVLILVVVLAVVVPRWRQQWSGPGTTKRFPETVLARCVKY
TEIHPEMRHVDCQSVWDAFKGAFISKHPCNITEEDYQPLMKLGTQTVPCNKILLWSRIKDLAHQFTQVQR
DMFTLEDTLLGYLADDLTWCGEFNTSKINYQSCPDWRKDCSNNPVSVFWKTVSRRFAEAACDVVHVMLNG
SRSKIFDKNSTFGSVEVHNLQPEKVQTLEAWVIHGGREDSRDLCQDPTIKELESIISKRNIQFSCKNIYR
PDKFLQCVKNPEDSSCTSEI

The present disclosure provides a number of CD19 target sites and corresponding gRNAs that are useful for targeting an RNA-guided nuclease to human CD19. Table 7 below illustrates preferred target domains in the human endogenous CD19 gene that can be bound by gRNAs described herein. The exemplary target sequences of human CD19 shown in Table 7, in some embodiments, are for use with a Cas9 nuclease, e.g., SpCas9.

TABLE 7
Exemplary Cas9 target site sequences of human CD19 are provided,
as are exemplary gRNA targeting domain sequences useful for
targeting such sites. For each target site, the first sequence
represents the DNA target domain sequence, the second sequence
represents the reverse complement thereof, and the third sequence
represents an exemplary targeting domain sequence of a gRNA that
can be used to target the respective target site.

| gRNA Name | Target domain sequence |
|---|---|
| CD19_K220_sg1 | CTGGACCCATGTGCACCCCAAGG (SEQ ID NO: 64) |
| | CCTTGGGGTGCACATGGGTCCAG (SEQ ID NO: 65) |
| | CUGGACCCAUGUGCACCCCAAGG (SEQ ID NO: 66) |
| CD19_K220_sg2 | TGGACCCATGTGCACCCCAAGGG (SEQ ID NO: 67) |
| | CCCTTGGGGTGCACATGGGTCCA (SEQ ID NO: 68) |
| | UGGACCCAUGUGCACCCCAAGGG (SEQ ID NO: 69) |
| CD19_K220 sg3 | GGACCCATGTGCACCCCAAGGGG (SEQ ID NO: 70) |
| | CCCCTTGGGGTGCACATGGGTCC (SEQ ID NO: 71) |
| | GGACCCAUGUGCACCCCAAGGGG (SEQ ID NO: 72) |
| CD19_K220_sg6 | CCAAGGGGCCTAAGTCATTGCTG (SEQ ID NO: 73) |
| | CAGCAATGACTTAGGCCCCTTGG (SEQ ID NO: 74) |
| | CCAAGGGGCCUAAGUCAUUGCUG (SEQ ID NO: 75) |
| CD19_K220_sg7 | CCCAAGGGGCCTAAGTCATTGCT (SEQ ID NO: 76) |
| | AGCAATGACTTAGGCCCCTTGGG (SEQ ID NO: 77) |
| | CCCAAGGGGCCUAAGUCAUUGCU (SEQ ID NO: 78) |
| CD19_K220_sg8 | CCCCAAGGGGCCTAAGTCATTGC (SEQ ID NO: 79) |
| | GCAATGACTTAGGCCCCTTGGGG (SEQ ID NO: 80) |
| | CCCCAAGGGGCCUAAGUCAUUGC (SEQ ID NO: 81) |
| CD19_K220_sg9 | CCATGTGCACCCCAAGGGGCCTA (SEQ ID NO: 82) |
| | TAGGCCCCTTGGGGTGCACATGG (SEQ ID NO: 83) |
| | CCAUGUGCACCCCAAGGGGCCUA (SEQ ID NO: 84) |

The present disclosure provides exemplary CD19 targeting gRNAs that are useful for targeting an RNA-guided nuclease to human CD19. Table 8 below illustrates preferred targeting domains for use in gRNAs targeting Cas9 nucleases to human endogenous CD19 gene. The exemplary target sequences of human CD19 shown in Table 8, in some embodiments, are for use with a Cas9 nuclease, e.g., SpCas9.

TABLE 8
Exemplary Cas9 targeting domain sequences of gRNAs targeted
to human CD19 are provided.

| gRNA Name | Targeting domain sequence |
|---|---|
| CD19_K220_sg1 | CUGGACCCAUGUGCACCCCAAGG (SEQ ID NO: 66) |
| CD19_K220_sg2 | UGGACCCAUGUGCACCCCAAGGG (SEQ ID NO: 69) |
| CD19_K220_sg3 | GGACCCAUGUGCACCCCAAGGGG (SEQ ID NO: 72) |
| CD19_K220_sg6 | CCAAGGGGCCUAAGUCAUUGCUG (SEQ ID NO: 75) |
| CD19_K220_sg7 | CCCAAGGGGCCUAAGUCAUUGCU (SEQ ID NO: 78) |
| CD19_K220_sg8 | CCCCAAGGGGCCUAAGUCAUUGC (SEQ ID NO: 81) |
| CD19_K220_sg9 | CCAUGUGCACCCCAAGGGGCCUA (SEQ ID NO: 84) |

TABLE 9
Exemplary CBE targeting domain sequences of gRNAs targeted to human CD19
are provided.

| | | Expected |
|---|---|---|
| gRNA Name | Targeting domain sequence | modification |
| CBE-CD19_sg1 | CAAAGACCGCCCTGAGATCT (SEQ ID NO: 117) | R163C |
| CBE_CD19_sg2 | AGACCGCCCTGAGATCTGGG (SEQ ID NO: 118) | R163C P164S |
| CBE_CD19_sg3 | GACCGCCCTGAGATCTGGGA (SEQ ID NO: 119) | R163C P164F |

A representative DNA sequence of CD19 gene is provided by NCBI Gene ID: 930, shown below.

(SEQ ID NO: 85)
AAACAGAAATGAACAAATAAACACACAAGATCATTTCCCGTGGTAGTGAGAGCTGGGATGAAAATAAAACAGCGT
GGCAGGGAGGAGGCAAGTGTTGTGAGTCTGGAGGGTTCCTGGAGAATGGGGCCTGAGGCGTGACCACCGCCTTCC
TCTCTGGGGGGACTGCCTGCCGCCCCCGCAGACACCCATGGTTGAGTGCCCTCCAGGCCCCTGCCTGCCCCAGCA
TCCCCTGCGCGAAGCTGGGTGCCCCGGAGAGTCTGACCACCATGCCACCTCCTCGCCTCCTCTTCTTCCTCCTCT
TCCTCACCCCCATGGAAGTCAGGCCCGAGGAACCTCTAGTGGTGAAGGTGGAAGGTATGTCCAAAGGGCAGAAAG
GGAAGGGATTGAGGCTGGAAACTTGAGTTGTGGCTGGGTGTCCTTGGCTGAGTAACTTACCCTCTCTGAGCCTCC
ATTTTCTTATTTGTAAAATTCAGGAAAGGGTTGGAAGGACTCTGCCGGCTCCTCCACTCCCAGCTTTTGGAGTCC
TCTGCTCTATAACCTGGTGTGAGGAGTCGGGGGGCTTGGAGGTCCCCCCCACCCATGCCCACACCTCTCTCCCTC
TCTCTCCACAGAGGGAGATAACGCTGTGCTGCAGTGCCTCAAGGGGACCTCAGATGGCCCCACTCAGCAGCTGAC
CTGGTCTCGGGAGTCCCCGCTTAAACCCTTCTTAAAACTCAGCCTGGGGCTGCCAGGCCTGGGAATCCACATGAG
GCCCCTGGCCATCTGGCTTTTCATCTTCAACGTCTCTCAACAGATGGGGGGCTTCTACCTGTGCCAGCCGGGGCC
CCCCTCTGAGAAGGCCTGGCAGCCTGGCTGGACAGTCAATGTGGAGGGCAGCGGTGAGGGCCGGGCTGGGGCAGG
GGCAGGAGGAGAGAAGGGAGGCCACCATGGACAGAAGAGGTCCGCGGCCACAATGGAGCTGGAGAGAGGGGCTGG
AGGGATTGAGGGCGAAACTCGGAGCTAGGTGGGCAGACTCCTGGGGCTTCGTGGCTTCAGTATGAGCTGCTTCCT
GTCCCTCTACCTCTCACTGTCTTCTCTCTCTCTGCGGGTCTTTGTCTCTATTTATCTCTGTCTTTGAGTCTCTAT
CTCTCTCCCTCTCCTGGGTGTCTCTGCATTTGGTTCTGGGTCTCTTCCCAGGGGAGCTGTTCCGGTGGAATGTTT
CGGACCTAGGTGGCCTGGGCTGTGGCCTGAAGAACAGGTCCTCAGAGGGCCCCAGCTCCCCTTCCGGGAAGCTCA
TGAGCCCCAAGCTGTATGTGTGGGCCAAAGACCGCCCTGAGATCTGGGAGGGAGAGCCTCCGTGTCTCCCACCGA
GGGACAGCCTGAACCAGAGCCTCAGCCAGGGTATGGTGATGACTGGGGAGATGCCGGGAAGCGGGGGTCCAGAGA
CAGAGGGGAGGGGAAACTGAAGAGGTGAAACCCTGAGGATCAGGCTTTCCTTGTCTTATCTCTCCCTGTCCCAGA
CCTCACCATGGCCCCTGGCTCCACACTCTGGCTGTCCTGTGGGGTACCCCCTGACTCTGTGTCCAGGGGCCCCCT
CTCCTGGACCCATGTGCACCCCAAGGGGCCTAAGTCATTGCTGAGCCTAGAGCTGAAGGACGATCGCCCGGCCAG
AGATATGTGGGTAATGGAGACGGGTCTGTTGTTGCCCCGGGCCACAGCTCAAGACGCTGGAAAGTATTATTGTCA
CCGTGGCAACCTGACCATGTCATTCCACCTGGAGATCACTGCTCGGCCAGGTAGAGTTTCTCTCAACTGGGAGGC
ATCTGTGTGGGGGTACTGGGAAGAAGTGGAAGCCAGTCAATCTTAGATTCCCCCAACCCGAGGGCTACTCCCAGC
CTCACCCCAAACCCCAACTTCCACACAGAACACTGACTCCAAGTCTTTCTTTTTTTTGACAGAGTCTCGCTCTGT
TGCCTAGGCTGGAGTGCAGTGGTGCCATCTTGTCTTGGCTCACTGCAACCTCCGCCTCCCAGGTTCAAGTGATTC
CCCTGCCTCAGCCTCCTGAGTAGCTGGGATTACAGGTGCCCACCACCACGCCTGGCTAATTTTTTTTTTTTTTTT
GAGACGGAGTCTTGCACTGTCACCCAGGCTGGAGTGCAGTGGCACGATCTCAGCTCACTGCAACCTCCACCTTCC
AGGTTCAAGTGATTCTCCTGCCTCAGCCTCCCGAGTAGCTGGGATTAAAGCCTGGCTAATTTTTTTTGTATTTTT
AGTAGAGATGGGGTTTCATTATGTTGGCCAGGCTGGTCTCAAACTCCTGACCTCGTGATCCACCCGCCTCGGCCT
CCCAAAGTGCTGGGATTACAGACATGAGCCACAGGGCCGGGCCAAGCCTAATTTTGTATTTTTAGTAGAGATGGG
GTTTCTCCCTGTTGGACCAGGCTGGTCTTGAACTCCTGACTTCAGGTGATCTGCCTGCCTTGGCCTCCCAAAGTA
CTGGGATTACAGGCATAAGCCACCGCACCTGGCCTAGACTTCAAGTCTTTCTTCCCTCGCTTCCAAGACACTACT
TTTCTGGGTCTTCACCTACCATTGCTTGCGCCTGCCCACCAGCTTGGGTGGAGTCTTCCTTCCTCCCCAACTCCT
CACTCTTGGAGCCCTGGGCCCTCTTCTTATCCCTGTCTGCACACTTTCCTATTTGAACTTGACTCTCAATGGCTT
CTTGGGTCACCATGCCTTGGTGACTCTATTCCAGGCTCCATACTCAGCCATCTCCTGTGCCATTTGATATCCCAT
GGACACCTCAGGCTCAACAGATACAAAATCAAACTCAATGTCTTCCCCAAGTATAGTCTTCTTGGTGGCCCAGTG
TAAGCAGAGGGCACCACCACCTGCTCCCTCGCCCAGGCTAAGAACCTGGGCATCCTTCTTTTTCCTCACCCCGTC
CAACAAACTGGTCACAGTGTTCTGCCAATTCTCTCTCCATGCAATCCTATCATGCTATCCTAACTGCAATTCACA
AACCCAACCCCAACTTTCACTCCAAACTTGATCCAAGCAATGTGCTGGATCCCAACTGTAACCTTGCAAACTCAA
CTCTGCCCTTCACTTTGACCGTGACTATCCTTAATTGCAGCAGGAAACTGATCATTATGCTCCCCTCAATCCACA
CATTGCCTCTGAGTACAGCCATGGTTTGTCCACGATTTGCTCAAAGACACTGCCCCATGTCCTGTGCCAGGGTCT
GTGACAATCCCTGACCTCCTGGGACATGGCTCCTTAGAGAGAGGAGAGCCTTTCTCACAGCTTGGGACTTTGAGT
CTGTGTCTTTTTTTTTTTCTTGAGACGGAGTTTTGCTGTGGTTGCCCAGGCTGGAGTGCAGTGATCTCGGCTCAC
TGAAACCTCCGCCTCCCGGGTTCAAACGATTCTCCTGCCTCAGCCTCCCAAGTAGCTGGGATTACAGGCACCCAC
CACCATGCCCAGCTAATTTTTTTGTATTTTTAGTAGAGATGGGGTTTCACCATGTTGGCCAGGCTGGTCTCGAAC
TCCTGACCTCAGGTGATCCACCCGCCTTTGCCTCCCAAAGTGCTGGGATTACAGGCGTCAACCACCGCGCCCGGC
CGAGTCTGTGTCTTGCCTCTGTGCCTCAGACTTGCGGTTCCTTGAGATCTCAGGATTGGGACGTAAGATGCCAGC
CTGGGGTCCTCGTCTCATAGCCCCTTCCCCCTAGTACTATGGCACTGGCTGCTGAGGACTGGTGGCTGGAAGGTC
TCAGCTGTGACTTTGGCTTATCTGATCTTCTGCCTGTGTTCCCTTGTGGGCATTCTTCATCTTCAAAGAGGTGAG
TCATGTCCCCAGTGGGTCTGTCCAAACCCTACTCCATCTTCCCCAGGATAAGCCGGCTCTGGCCAGTCTGACAAC
CATCTTTCTTTCCTCCCATCCCTCCCTTCAAGACCCCAGAATCCTGTTCTCCCCAGTCTTCCTCTAGCCTCCCTC
AAACTTCCCAAGCCTCTTGCAATTTTTTTTTTTTTTTTGAGACAGGGTCTCATTCTGTCACCCCAGCTGGAGTGC
AGTGGCACAATCTGAGCTCACTGTAACCTCTGCCTCCCAGGCTTAAGTGATTCTTGTGCTTCAGCCTCCCGAGTA
CCTGGGACTACAAGTGTATGCCACCACACCCGGCCAATTTTTTATATTTTTAGTAGAGACGAGGTTTCACCATGT
TGGCCAGACTGGTCTCGAACTCTTGACCTCAAATGATCCGCCCACCTCGGCCTCCCAAAGTGCTGGGATTACAGG
CACGAGCCACCGCGCCCGTCCGCCTCGCAATTTGAACTCCTGTCTCCTTTGTTGAACCAAGTGACCTCCCCAGCA
CCTGGCCCCACAAATCCTCACCCTGCCAAGCAGCCCCTCCTCTGATCACGCCCTTTAACTCCCACCAGCCCTGGT
CCTGAGGAGGAAAAGAAAGCGAATGACTGACCCCACCAGGAGGTAATGCAACCAGTGCACCCCGCGGTAACACCC
TCCACCTTCACTTTATGCCTTGCACTTACTGTTTCCTCTGCCCAGGGGTTCTTTGCTCCGTCTCTACTGTTTCAA
ATACTGCCCAACCTCAAAGCCCAGCTCCAAAGCTACCTCCTCTGTGAAGAACTCCTTGGAAATGATCATCTCAGA
CTCCTCTATTGGCTGTCCCAGCACAAGTGATCACGTTTAACTTCTGAAGGCCTGGACAGAATCTTGAGTGGGTCC
GCCATTCCATTCCAAGTCGGCCCTCACCGTGCACTTCCTCTTCTCCCGCCAGATTCTTCAAAGTGACGCCTCCCC
CAGGAAGCGGGCCCCAGAACCAGTACGGGAACGTGCTGTCTCTCCCCACACCCACCTCAGGCCTCGGTAAGAGGC
ACCGCCCCTCCAGCCTATAGCTCCGCCCCAGATCCGGGGCTCCACCCCCACTCTCCTCATCCCTCCAATCCGCTG
TGCGCCAAGCCTTCTGGAGCTCGGAACTCCGCCCCCGGGGCGGGGAGTCCCGCCCAGCTATGAGCCCCGCCTCTA
GAACCAGACCCCGCCTCCAGGGCTCAGAGCCACGCCCCCAGGACCCAGAGCCTGAAGTCGTAATCAAGAGCAGAA
CTTCGCCCCAGAACTGAAGGCCTCGGCCCTAGATTTAGATTCCGCCCCAGGGTTCAAGGCCGGGTTCCTAGACCC
AGAGTCCATTCGCAGAGCCCAAAACATCCTCTTCCCGTGCCCCGCCGCGCGGACCCTTAGCCTTGACCGCCCCCA
TCTCTTCTGACCCCGTCTTACAATGCCCCTCTCACCAGGACGCGCCCAGCGTTGGGCCGCAGGCCTGGGGGGCAC
TGCCCCGTCTTATGGAAACCCGAGCAGCGACGTCCAGGCGGATGGAGCCTTGGGGTCCCGGAGCCCGCCGGGAGT
GGGTGAATGACTGGGAGAGGGAAGGGTCGTTCCCCACATGGAGGGGGTTGGAGCGGTCTGTGGCCCGAATAGTGG
ACTGGGCCCTGGAGGAGAGGGGGCATGACTCGGTTCCCCATCCCCATCCCCAAACCCCCAGGCCCAGAAGAAGAG
GAAGGGGAGGGCTATGAGGAACCTGACAGTGAGGAGGACTCCGAGTTCTATGAGAACGACTCCAACCTTGGGCAG
GACCAGCTCTCCCAGGGTAAGGCTGCCCTCCCCCGTGGCCCCCCACCTCTGCGGTGGCCTGTGGACTCCCATGGA
CACCCCTCCTTCTCCACCAGATGGCAGCGGCTACGAGAACCCTGAGGATGAGCCCCTGGGTCCTGAGGATGAAGA
CTCCTTCTCCAACGGTAACTTGGGGCCTTTGTGGGACCTCAGAGACTTAGGTGTAATTGCAGCGCTGTGACACTC
CTAGAAGGGGTCCCTGGAGTTCTCTCTCTTCTGCCACAGCTGAGTCTTATGAGAACGAGGATGAAGAGCTGACCC
AGCCGGTCGCCAGGACAATGGGTGTGTGTGAGGATGGCAACAGTCCAGGGGGGAGGCGGAGGACACCTGGAGGCC
AGGAGGAATAGTAACCTCCCTCTTCCCTTTCCAGACTTCCTGAGCCCTCATGGGTCAGCCTGGGACCCCAGCCGG
GAAGCAACCTCCCTGGGTGAGAGATGCTTTCAATCAGACTGCCTTGCCCAGCTTGGGTGACCTGGCCTCAGCTCT
GACACCAGATCCAACTTTGACCTGACCCTGACCCCAAACCCGAACCCAATCCTGTGACTCCTCTCACCTCAACAC
TGAGCCCCATCCCCCATCCTGAGCCCCATCCCCCATCCTGACCCCCAATATTTACCCCCTCCCTAACTGTGAATA
TCAACACCGATCCCAATGCAGTATCAGCCTGGACTTGATCTCCACCTCACCTCAGCCCCAGTGCAGACCTCAACT
TGGACCCCAGCTTACTCTGCAGCTTCTTCATGACTCTGACTCCGACTCCCTCCAGTTTCTTCTTTTTCTTTTTCT
TTTTTTTGAGACGGAGTCTCCCTCTGTTGCCCAGGCTGGAGTGCAGTTGCCACCTCTGCCTCCTAGGTTCAAGCG
ATTCTCATGCCTCAGCCTCCTGAGTAGCTGGGATTATAGACGTTTGCCACCACACCTGGCTAATTTTTGTATTTT
CAGTAGAGACAGGGTTTCGCCATGTTGGCCAGACTGGTCTCCAACTCCTGGCCTCTAGTGATCTGCCCGCCTTGG
CTTCCCAAAGTGCTGGGATTACAGGCATGAGCCACCACGCCCAGCCCAGTTCTGTTCTTGACCCCTTCCTTAGCC
ATAATCTAACCCATATCTAACCCTGACCCTACAGCTAACTGGGGCCCCAAACTCAATGCTAACCAAATCACCCCT
TCCCAGCACAGCATGGGTAATGCTCCTCACCTTCCTCTGCCCCTCAGTCTTCCTCCTTACCGTAGGCTGTACTTC
CCATGCCCTAGCCTCCAATTCTCCATCCCCCGCCCAAGCAGGGTCCCAGTCCTATGAGGATATGAGAGGAATCCT
GTATGCAGCCCCCCAGCTCCGCTCCATTCGGGGCCAGCCTGGACCCAATCATGAGGAAGGTGGGTGCTTCTGCCG
CTGTCCCCTGCTGTCCCCTGGGCTGACTTTGCCTTCCAGCCTACTTCCAGTGCCACCCATGTTCTCCTCCTCCCT
GGTCCTATCCAGATGCAGACTCTTATGAGAACATGGATAATCCCGATGGGCCAGACCCAGCCTGGGGAGGAGGGG
GCCGCATGGGCACCTGGAGCACCAGGTGATCCTCAGGTGGCCAGGTGAGCTGGGACTGCCCCTAGGGAAAGCGGG
GAGGGAGGGAGATAGGCACGGATGGCAGTGGCTGCTGGCTTTCAGGGAGGGAGAGGGAACAGGGTTCCTAGGGCC
TGGTGGGCAGGGGGAGGACTGCTGGACCCCTCCCCATCACCGTTTCTTCTGCATAGCCTGGATCTCCTCAAGTCC
CCAAGATTCACACCTGACTCTGAAATCTGAAGACCTCGAGCAGATGATGCCAACCTCTGGAGCAATGTTGCTTAG
GATGTGTGCATGTGTGTAAGTGTGTGTGTGTGTGTGTGTGTGTATACATGCCAGTGACACTTCCAGTCCCCTTTG
TATTCCTTAAATAAACTCAATGAGCTCTTCCAATCCTA

A representative mRNA sequence of CD19 (transcript variant 2) is provided by NCBI Reference Sequence No: NM_001770.6, shown below:

| (SEQ ID NO: 86) | |
|---|---|
| 1 | aagctgggtg ccccggagag tctgaccacc atgccacctc ctcgcctcct cttcttcctc |
| 61 | ctcttcctca cccccatgga agtcaggccc gaggaacctc tagtggtgaa ggtggaagag |
| 121 | ggagataacg ctgtgctgca gtgcctcaag gggacctcag atggccccac tcagcagctg |
| 181 | acctggtctc gggagtcccc gcttaaaccc ttcttaaaac tcagcctggg gctgccaggc |
| 241 | ctgggaatcc acatgaggcc cctggccatc tggcttttca tcttcaacgt ctctcaacag |
| 301 | atggggggct tctacctgtg ccagccgggg cccccctctg agaaggcctg gcagcctggc |
| 361 | tggacagtca atgtggaggg cagcggggag ctgttccggt ggaatgtttc ggacctaggt |
| 421 | ggcctgggct gtggcctgaa gaacaggtcc tcagagggcc ccagctcccc ttccgggaag |
| 481 | ctcatgagcc ccaagctgta tgtgtgggcc aaagaccgcc ctgagatctg ggagggagag |
| 541 | cctccgtgtc tcccaccgag ggacagcctg aaccagagcc tcagccagga cctcaccatg |
| 601 | gcccctggct ccacactctg gctgtcctgt ggggtacccc ctgactctgt gtccaggggc |
| 661 | cccctctcct ggacccatgt gcaccccaag gggcctaagt cattgctgag cctagagctg |
| 721 | aaggacgatc gcccggccag agatatgtgg gtaatggaga cgggtctgtt gttgccccgg |
| 781 | gccacagctc aagacgctgg aaagtattat tgtcaccgtg gcaacctgac catgtcattc |
| 841 | cacctggaga tcactgctcg gccagtacta tggcactggc tgctgaggac tggtggctgg |
| 901 | aaggtctcag ctgtgacttt ggcttatctg atcttctgcc tgtgttccct tgtgggcatt |
| 961 | cttcatcttc aaagagccct ggtcctgagg aggaaaagaa agcgaatgac tgaccccacc |
| 1021 | aggagattct tcaaagtgac gcctccccca ggaagcgggc cccagaacca gtacgggaac |
| 1081 | gtgctgtctc tccccacacc cacctcaggc ctcggacgcg cccagcgttg ggccgcaggc |
| 1141 | ctggggggca ctgccccgtc ttatggaaac ccgagcagcg acgtccaggc ggatggagcc |
| 1201 | ttggggtccc ggagcccgcc gggagtgggc ccagaagaag aggaagggga gggctatgag |
| 1261 | gaacctgaca gtgaggagga ctccgagttc tatgagaacg actccaacct tgggcaggac |
| 1321 | cagctctccc aggatggcag cggctacgag aaccctgagg atgagcccct gggtcctgag |
| 1381 | gatgaagact ccttctccaa cgctgagtct tatgagaacg aggatgaaga gctgacccag |
| 1441 | ccggtcgcca ggacaatgga cttcctgagc cctcatgggt cagcctggga ccccagccgg |
| 1501 | gaagcaacct ccctggggtc ccagtcctat gaggatatga gaggaatcct gtatgcagcc |
| 1561 | ccccagctcc gctccattcg gggccagcct ggacccaatc atgaggaaga tgcagactct |
| 1621 | tatgagaaca tggataatcc cgatgggcca gacccagcct ggggaggagg gggccgcatg |
| 1681 | ggcacctgga gcaccaggtg atcctcaggt ggccagcctg gatctcctca agtccccaag |
| 1741 | attcacacct gactctgaaa tctgaagacc tcgagcagat gatgccaacc tctggagcaa |
| 1801 | tgttgcttag gatgtgtgca tgtgtgtaag tgtgtgtgtg tgtgtgtgtg tgtatacatg |
| 1861 | ccagtgacac ttccagtccc ctttgtattc cttaaataaa ctcaatgagc tcttccaa |

A representative amino acid sequence of CD19 is provided by NCBI Reference Sequence No. NP_001761.3, shown below.

(SEQ ID NO: 87)
MPPPRLLFFLLFLTPMEVRPEEPLVVKVEEGDNAVLQCLKGTSDGPTQQLTWSRESPLKPFLKLSLGLPG
LGIHMRPLAIWLFIFNVSQOMGGFYLCQPGPPSEKAWQPGWTVNVEGSGELFRWNVSDLGGLGCGLKNRS
SEGPSSPSGKLMSPKLYVWAKDRPEIWEGEPPCLPPRDSLNQSLSQDLTMAPGSTLWLSCGVPPDSVSRG
PLSWTHVHPKGPKSLLSLELKDDRPARDMWVMETGLLLPRATAQDAGKYYCHRGNLTMSFHLEITARPVL
WHWLLRTGGWKVSAVTLAYLIFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVGPEEEEGEGYEEPDSEEDSEF
YENDSNLGQDQLSQDGSGYENPEDEPLGPEDEDSFSNAESYENEDEELTQPVARTMDFLSPHGSAWDPSR
EATSLGSQSYEDMRGILYAAPQLRSIRGOPGPNHEEDADSYENMDNPDGPDPAWGGGGRMGTWSTR

The present disclosure provides a number of EMR2 target sites and corresponding gRNAs that are useful for targeting an RNA-guided nuclease to human EMR2. Tables 10 and 11 below illustrates preferred target domains in the human endogenous ADGRE2 gene coding EMR2 (CD312) that can be bound by gRNAs described herein. The exemplary target sequences of human EMR2 shown in Tables 7 and 8, in some embodiments, are for use with a base editor, e.g., CBE or ABE.

Tables 10 and 11. Exemplary target site sequences of human EMR2 are provided, as are exemplary gRNA targeting domain sequences useful for targeting such sites. For each target site, the first sequence represents the DNA target domain sequence, the second sequence represents the reverse complement thereof, and the third sequence represents an exemplary targeting domain sequence of a gRNA that can be used to target the respective target site.

TABLE 10
Exemplary sequences of gRNAs targeted to human EMR2 are provided.

| | | | | | CBE | ABE |
|---|---|---|---|---|---|---|
| Guide | Amino | | | | resulting | resulting |
| Name | Acid | Target Domain Sequence | Exon | Codon | edit | edit |
| EMR2-1 | 292 | CAGAGACTACAAGCCAGGCT (SEQ ID NO: 286) | 10 | TAC | GAGAcTA | GaGaCTa |
| | | AGCCTGGCTTGTAGTCTCTG (SEQ ID NO: 287) | | | | |
| | | CAGAGACUACAAGCCAGGCU (SEQ ID NO: 288) | | | | |
| EMR2-2 | 292 | GAGACTACAAGCCAGGCTTG (SEQ ID NO: 289) | 10 | TAC | GAcTAcA | GaCTaCa |
| | | CAAGCCTGGCTTGTAGTCTC (SEQ ID NO: 290) | | | | |
| | | GAGACUACAAGCCAGGCUUG (SEQ ID NO: 291) | | | | |
| EMR2-3 | 296 | GGCtTGGCCAATAACACCAT (SEQ ID NO: 292) | 10 | TTG | cTTGGc | CTTGGC |
| | | ATGGTGTTATTGGCCAaGCC (SEQ ID NO: 293) | | | | |
| | | GGCuUGGCCAAUAACACCAU (SEQ ID NO: 294) | | | | |
| EMR2-4 | 298 | CTTGGCCaATAACACCATCC (SEQ ID NO: 295) | 10 | AAT | TGGccA | TGGCCa |
| | | GGATGGTGTTATtGGCCAAG (SEQ ID NO: 296) | | | | |
| | | CUUGGCCaAUAACACCAUCC (SEQ ID NO: 297) | | | | |
| EMR2-5 | 299 | CCAATAACACCATCCAGGTA (SEQ ID NO: 298) | 10 | AAC | AATAAcA | aaTaaCa |
| | | TACCTGGATGGTGTTATTGG (SEQ ID NO: 299) | | | | |
| | | CCAAUAACACCAUCCAGGUA (SEQ ID NO: 300) | | | | |
| EMR2-6 | 299 | CAATAACACCATCCAGGTAA (SEQ ID NO: 301) | 10 | AAC | ATAAcAc | aTaaCaC |
| | | TTACCTGGATGGTGTTATTG (SEQ ID NO: 302) | | | | |
| | | CAAUAACACCAUCCAGGUAA (SEQ ID NO: 303) | | | | |
| EMR2-7 | 303 | CCAGAGCATCTTACAGGCGC (SEQ ID NO: 304) | 11 | AGC | AGAGcAT | aGaGCaT |
| | | GCGCCTGTAAGATGCTCTGG (SEQ ID NO: 305) | | | | |
| | | CCAGAGCAUCUUACAGGCGC (SEQ ID NO: 306) | | | | |
| EMR2-8 | 303 | CAGAGCATCTTACAGGCGCT (SEQ ID NO: 307) | 11 | AGC | GAGcATc | GaGCaTC |
| | | AGCGCCTGTAAGATGCTCTG (SEQ ID NO: 308) | | | | |
| | | CAGAGCAUCUUACAGGCGCU (SEQ ID NO: 309) | | | | |
| EMR2-9 | 303 | TCCAGAGCATCTTACAGGCG (SEQ ID NO: 310) | 11 | AGC | cAGAGcA | CaGaGCa |
| | | CGCCTGTAAGATGCTCTGGA (SEQ ID NO: 311) | | | | |
| | | UCCAGAGCAUCUUACAGGCG (SEQ ID NO: 312) | | | | |
| EMR2-10 | 303 | TCCAGAGCATCTTACAGGCG (SEQ ID NO: 310) | 11 | AGC | cAGAGcA | CaGaGCa |
| | | CGCCTGTAAGATGCTCTGGA (SEQ ID NO: 311) | | | | |
| | | UCCAGAGCAUCUUACAGGCG (SEQ ID NO: 312) | | | | |
| EMR2-11 | 304 | CCAGAGCaTCTTACAGGCGC (SEQ ID NO: 304) | 11 | ATC | AGAGcA | aGaGCa |
| | | GCGCCTGTAAGAtGCTCTGG (SEQ ID NO: 305) | | | | |
| | | CCAGAGCaUCUUACAGGCGC (SEQ ID NO: 306) | | | | |
| EMR2-12 | 304 | CAGAGCaTCTTACAGGCGCT (SEQ ID NO: 307) | 11 | ATC | GAGcAT | GaGCaT |
| | | AGCGCCTGTAAGAtGCTCTG (SEQ ID NO: 308) | | | | |
| | | CAGAGCaUCUUACAGGCGCU (SEQ ID NO: 309) | | | | |
| EMR2-13 | 304 | AGCaTCTTACAGGCGCTGGA (SEQ ID NO: 313) | 11 | ATC | cATcTT | CaTCTT |
| | | TCCAGCGCCTGTAAGAtGCT (SEQ ID NO: 314) | | | | |
| | | AGCaUCUUACAGGCGCUGGA (SEQ ID NO: 315) | | | | |
| EMR2-14 | 305 | AGCATCtTACAGGCGCTGGA (SEQ ID NO: 313) | 11 | TTA | cATcTT | CaTCTT |
| | | TCCAGCGCCTGTAaGATGCT (SEQ ID NO: 314) | | | | |
| | | AGCAUCuUACAGGCGCUGGA (SEQ ID NO: 315) | | | | |
| EMR2-15 | 305 | AGCATCtTACAGGCGCTGGA (SEQ ID NO: 313) | 11 | TTA | cATcTT | CaTCTT |
| | | TCCAGCGCCTGTAaGATGCT (SEQ ID NO: 314) | | | | |
| | | AGCAUCuUACAGGCGCUGGA (SEQ ID NO: 315) | | | | |
| EMR2-16 | 307 | ACAGgCGCTGGATGAGCTGC (SEQ ID NO: 316) | 11 | GCG | AGGcGc | aGGCGC |
| | | GCAGCTCATCCAGCGcCTGT (SEQ ID NO: 317) | | | | |
| | | ACAGgCGCUGGAUGAGCUGC (SEQ ID NO: 318) | | | | |
| EMR2-17 | 307 | CAGgCGCTGGATGAGCTGCT (SEQ ID NO: 319) | 11 | GCG | GGcGcT | GGCGCT |
| | | AGCAGCTCATCCAGCGcCTG (SEQ ID NO: 320) | | | | |
| | | CAGgCGCUGGAUGAGCUGCU (SEQ ID NO: 321) | | | | |
| EMR2-18 | 307 | CAGgCGCTGGATGAGCTGCT (SEQ ID NO: 319) | 11 | GCG | GGcGcT | GGCGCT |
| | | AGCAGCTCATCCAGCGcCTG (SEQ ID NO: 320) | | | | |
| | | CAGgCGCUGGAUGAGCUGCU (SEQ ID NO: 321) | | | | |
| EMR2-19 | 308 | GGCGcTGGATGAGCTGCTGG (SEQ ID NO: 322) | 11 | CTG | cGcTGG | CGCTGG |
| | | CCAGCAGCTCATCCAgCGCC (SEQ ID NO: 323) | | | | |
| | | GGCGcUGGAUGAGCUGCUGG (SEQ ID NO: 324) | | | | |
| EMR2-20 | 308 | CAGGCGcTGGATGAGCTGCT (SEQ ID NO: 319) | 11 | CTG | GGcGcT | GGCGCT |
| | | AGCAGCTCATCCAgCGCCTG (SEQ ID NO: 320) | | | | |
| | | CAGGCGcUGGAUGAGCUGCU (SEQ ID NO: 321) | | | | |
| EMR2-21 | 308 | CAGGCGcTGGATGAGCTGCT (SEQ ID NO: 319) | 11 | CTG | GGcGcT | GGCGCT |
| | | AGCAGCTCATCCAgCGCCTG (SEQ ID NO: 320) | | | | |
| | | CAGGCGcUGGAUGAGCUGCU (SEQ ID NO: 321) | | | | |
| EMR2-22 | 312 | TGAGCTGcTGGAGGCCCCTG (SEQ ID NO: 325) | 11 | CTG | AGcTGcT | aGCTGCT |
| | | CAGGGGCCTCCAgCAGCTCA (SEQ ID NO: 326) | | | | |
| | | UGAGCUGcUGGAGGCCCCUG (SEQ ID NO: 327) | | | | |
| EMR2-23 | 312 | GAGCTGcTGGAGGCCCCTGG (SEQ ID NO: 328) | 11 | CTG | GcTGcTG | GCTGCTG |
| | | CCAGGGGCCTCCAgCAGCTC (SEQ ID NO: 329) | | | | |
| | | GAGCUGcUGGAGGCCCCUGG (SEQ ID NO: 330) | | | | |
| EMR2-24 | 329 | CTGTGTGGCCAGTCACCTGC (SEQ ID NO: 331) | 11 | GTG | GTGTGGc | GTGTGGC |
| | | GCAGGTGACTGGCCACACAG (SEQ ID NO: 332) | | | | |
| | | CUGUGUGGCCAGUCACCUGC (SEQ ID NO: 333) | | | | |
| EMR2-25 | 329 | TGTgTGGCCAGTCACCTGCT (SEQ ID NO: 334) | 11 | GTG | TGTGGcc | TGTGGCC |
| | | AGCAGGTGACTGGCCAcACA (SEQ ID NO: 335) | | | | |
| | | UGUgUGGCCAGUCACCUGCU (SEQ ID NO: 336) | | | | |
| EMR2-26 | 329 | ACTGTgTGGCCAGTCACCTG (SEQ ID NO: 337) | 11 | GTG | TGTGTGG | TGTGTGG |
| | | CAGGTGACTGGCCAcACAGT (SEQ ID NO: 338) | | | | |
| | | ACUGUgUGGCCAGUCACCUG (SEQ ID NO: 339) | | | | |
| EMR2-27 | 332 | AGTCACCTGCTGGATGGCCT (SEQ ID NO: 340) | 11 | CAC | TcAccTG | TCaCCTG |
| | | AGGCCATCCAGCAGGTGACT (SEQ ID NO: 341) | | | | |
| | | AGUCACCUGCUGGAUGGCCU (SEQ ID NO: 342) | | | | |
| EMR2-28 | 332 | AGTCACCTGCTGGATGGCCT (SEQ ID NO: 340) | 11 | CAC | TcAccTG | TCaCCTG |
| | | AGGCCATCCAGcAGGTGACT (SEQ ID NO: 341) | | | | |
| | | AGUCACCUGCUGGAUGGCCU (SEQ ID NO: 342) | | | | |
| EMR2-29 | 340 | AGAGGATgTCCTCAGAGGCC (SEQ ID NO: 343) | 11 | GTC | AGGATGT | aGGaTGT |
| | | GGCCTCTGAGGAcATCCTCT (SEQ ID NO: 344) | | | | |

| | | AGAGGAUgUCCUCAGAGGCC (SEQ ID NO: 345) | | | | |
|---|---|---|---|---|---|---|
| EMR2-30 | 340 | AGAGGATgTCCTCAGAGGCC (SEQ ID NO: 343) | 11 | GTC | AGGATGT | aGGaTGT |
| | | GGCCTCTGAGGAcATCCTCT (SEQ ID NO: 344) | | | | |

| | | | | | | |
|---|---|---|---|---|---|---|
| | | AGAGGAUgUCCUCAGAGGCC (SEQ ID NO: 345) | | | | |
| EMR2-31 | 527 | CCCACtACGATGTGCAGGTG (SEQ ID NO: 346) | 14 | TAC | cACTAcG | CaCTaCG |
| | | CACCTGCACATCGTaGTGGG (SEQ ID NO: 347) | | | | |
| | | CCCACuACGAUGUGCAGGUG (SEQ ID NO: 348) | | | | |
| EMR2-32 | 527 | GGCCCACtACGATGTGCAGG (SEQ ID NO: 349) | 14 | TAC | cccAcTA | CCCaCTa |
| | | CCTGCACATCGTaGTGGGCC (SEQ ID NO: 350) | | | | |
| | | GGCCCACuACGAUGUGCAGG (SEQ ID NO: 351) | | | | |
| EMR2-33 | 527 | GTCTCACCTGCACATCGTAG (SEQ ID NO: 352) | 14 | TAC | | |
| | | CTACGATGTGCAGGTGAGAC (SEQ ID NO: 353) | | | | |
| | | GUCUCACCUGCACAUCGUAG (SEQ ID NO: 354) | | | | |
| EMR2-34 | 527 | TCTCACCTGCACATCGTAGT (SEQ ID NO: 355) | 14 | TAC | | |
| | | ACTACGATGTGCAGGTGAGA (SEQ ID NO: 356) | | | | |
| | | UCUCACCUGCACAUCGUAGU (SEQ ID NO: 357) | | | | |
| EMR2-35 | 708 | CTCTCtGGATTTTGAAAAAC (SEQ ID NO: 358) | 18 | TGG | cTcTGGA | CTCTGGa |
| | | GTTTTTCAAAATCCaGAGAG (SEQ ID NO: 359) | | | | |
| | | CUCUCuGGAUUUUGAAAAAC (SEQ ID NO: 360) | | | | |

In Table 10, the lower case nucleotide refers to the edited nucleotide.

TABLE 11
Exemplary sequences of “PAMless” gRNAs targeted to human EMR2 are provided.

| Guide | Amino | | | | CBE | ABE |
|---|---|---|---|---|---|---|
| | | | | | resulting | resulting |
| Name | Acid | Target Domain Sequence | Exon | Codon | edit | edit |
| EMR2-36 | 292 | GAGACtACAAGCCAGGCTTG (SEQ ID NO: 289) | 10 | TAC | GACTACA | GaCTaCa |
| | | CAAGCCTGGCTTGTAGTCTC (SEQ ID NO: 290) | | | | |
| | | GAGACuACAAGCCAGGCUUG (SEQ ID NO: 291) | | | | |
| EMR2-37 | 294 | ACAAGCCAGGCTTGGCCAAT (SEQ ID NO: 361) | 10 | CCA | AAGccA | aaGCCa |
| | | ATTGGCCAAGCCTGgCTTGT (SEQ ID NO: 362) | | | | |
| | | ACAAGCCAGGCUUGGCCAAU (SEQ ID NO: 363) | | | | |
| EMR2-38 | 295 | AGCCAgGCTTGGCCAATAAC (SEQ ID NO: 364) | 10 | GGC | CCAGGc | CCaGGC |
| | | GTTATTGGCCAAGCCTGGCT (SEQ ID NO: 365) | | | | |
| | | AGCCAgGCUUGGCCAAUAAC (SEQ ID NO: 366) | | | | |
| EMR2-39 | 296 | CAGGCtTGGCCAATAACACC (SEQ ID NO: 367) | 10 | TTG | GGcTTGG | GGCTTGG |
| | | GGTGTTATTGGCCAAGCCTG (SEQ ID NO: 368) | | | | |
| | | CAGGCuUGGCCAAUAACACC (SEQ ID NO: 369) | | | | |
| EMR2-40 | 298 | TGGCCaATAACACCATCCAG (SEQ ID NO: 370) | 10 | AAT | GccAAT | GCCaaT |
| | | CTGGATGGTGTTATtGGCCA (SEQ ID NO: 371) | | | | |
| | | UGGCCaAUAACACCAUCCAG (SEQ ID NO: 372) | | | | |
| EMR2-41 | 299 | CCAATaACACCATCCAGGTA (SEQ ID NO: 298) | 10 | AAC | AATAAcA | aaTaaCa |
| | | TACCTGGATGGTGTtATTGG (SEQ ID NO: 299) | | | | |
| | | CCAAUaACACCAUCCAGGUA (SEQ ID NO: 300) | | | | |
| EMR2-42 | 303 | TCCAGaGCATCTTACAGGCG (SEQ ID NO: 310) | 11 | AGC | cAGAGcA | CaGaGCa |
| | | CGCCTGTAAGATGCTCTGGA (SEQ ID NO: 311) | | | | |
| | | UCCAGaGCAUCUUACAGGCG (SEQ ID NO: 312) | | | | |
| EMR2-43 | 304 | AGAGCaTCTTACAGGCGCTG (SEQ ID NO: 373) | 11 | ATC | AGcATc | aGCaTC |
| | | CAGCGCCTGTAAGAtGCTCT (SEQ ID NO: 374) | | | | |
| | | AGAGCaUCUUACAGGCGCUG (SEQ ID NO: 375) | | | | |
| EMR2-44 | 305 | GCATCtTACAGGCGCTGGAT (SEQ ID NO: 376) | 11 | TTA | ATcTTA | aTCTTa |
| | | ATCCAGCGCCTGTAaGATGC (SEQ ID NO: 377) | | | | |
| | | GCAUCuUACAGGCGCUGGAU (SEQ ID NO: 378) | | | | |
| EMR2-45 | 306 | TCTTACAGGCGCTGGATGAG (SEQ ID NO: 379) | 11 | CAG | TTAcAGG | TTaCaGG |
| | | CTCATCCAGCGCCTGTAAGA (SEQ ID NO: 380) | | | | |
| | | UCUUAcAGGCGCUGGAUGAG (SEQ ID NO: 381) | | | | |
| EMR2-46 | 307 | TACAGgCGCTGGATGAGCTG (SEQ ID NO: 382) | 11 | GCG | cAGGcG | CaGGCG |
| | | CAGCTCATCCAGCGCCTGTA (SEQ ID NO: 383) | | | | |
| | | UACAGgCGCUGGAUGAGCUG (SEQ ID NO: 384) | | | | |
| EMR2-47 | 308 | AGGCGCTGGATGAGCTGCTG (SEQ ID NO: 385) | 11 | CTG | GcGcTG | GCGCTG |
| | | CAGCAGCTCATCCAgCGCCT (SEQ ID NO: 386) | | | | |
| | | AGGCGCUGGAUGAGCUGCUG (SEQ ID NO: 387) | | | | |
| EMR2-48 | 312 | AGCTGCTGGAGGCCCCTGGG (SEQ ID NO: 388) | 11 | CTG | cTGcTGG | CTGCTGG |
| | | CCCAGGGGCCTCCAGCAGCT (SEQ ID NO: 389) | | | | |
| | | AGCUGCUGGAGGCCCCUGGG (SEQ ID NO: 390) | | | | |
| EMR2-49 | 318 | GGGACCTGGAGACCCTGCCC (SEQ ID NO: 391) | 11 | CTG | GAccTGG | GaCCTGG |
| | | GGGCAGGGTCTCCAGGTCCC (SEQ ID NO: 392) | | | | |
| | | GGGACCUGGAGACCCUGCCC (SEQ ID NO: 393) | | | | |
| EMR2-50 | 320 | TGGAGaCCCTGCCCCGCTTA (SEQ ID NO: 394) | 11 | ACC | GAGAccc | GaGaCCC |
| | | TAAGCGGGGCAGGGTCTCCA (SEQ ID NO: 395) | | | | |
| | | UGGAGaCCCUGCCCCGCUUA (SEQ ID NO: 396) | | | | |
| EMR2-51 | 328 | AGCACtGTGTGGCCAGTCAC (SEQ ID NO: 397) | 11 | TGT | cACTGTG | CaCTGTG |
| | | GTGACTGGCCACACaGTGCT (SEQ ID NO: 398) | | | | |
| | | AGCACuGUGUGGCCAGUCAC (SEQ ID NO: 399) | | | | |
| EMR2-52 | 329 | ACTGTgTGGCCAGTCACCTG (SEQ ID NO: 337) | 11 | GTG | TGTGTGG | TGTGTGG |
| | | CAGGTGACTGGCCACACAGT (SEQ ID NO: 338) | | | | |
| | | ACUGUgUGGCCAGUCACCUG (SEQ ID NO: 339) | | | | |
| EMR2-53 | 331 | TGGCCaGTCACCTGCTGGAT (SEQ ID NO: 400) | 11 | AGT | GccAGTc | GCCaGTC |
| | | ATCCAGCAGGTGACEGGCCA (SEQ ID NO: 401) | | | | |
| | | UGGCCaGUCACCUGCUGGAU (SEQ ID NO: 402) | | | | |
| EMR2-54 | 332 | CCAGTcACCTGCTGGATGGC (SEQ ID NO: 403) | 11 | CAC | AGTcAcc | aGTCaCC |
| | | GCCATCCAGCAGGTgACTGG (SEQ ID NO: 404) | | | | |
| | | CCAGUCACCUGCUGGAUGGC (SEQ ID NO: 405) | | | | |
| EMR2-55 | 335 | TGCTGgATGGCCTAGAGGAT (SEQ ID NO: 406) | 11 | GAT | CTGGATG | CTGGaTG |
| | | ATCCTCTAGGCCATCCAGCA (SEQ ID NO: 407) | | | | |
| | | UGCUGgAUGGCCUAGAGGAU (SEQ ID NO: 408) | | | | |
| EMR2-56 | 340 | AGGATgTCCTCAGAGGCCTG (SEQ ID NO: 409) | 11 | GTC | GATGTcc | GaTGTCC |
| | | CAGGCCTCTGAGGAcATCCT (SEQ ID NO: 410) | | | | |
| | | AGGAUgUCCUCAGAGGCCUG (SEQ ID NO: 411) | | | | |
| EMR2-57 | 347 | GCAAGaACCTTTCCAATGGG (SEQ ID NO: 412) | 11 | AAC | AAGAAcc | aaGaaCC |
| | | CCCATTGGAAAGGTtCTTGC (SEQ ID NO: 413) | | | | |
| | | GCAAGaACCUUUCCAAUGGG (SEQ ID NO: 414) | | | | |
| EMR2-58 | 124 | AATGTCAGCAGAACCCAAGG (SEQ ID NO: 415) | 6 | CAG | TGTcAGc | TGTCaGC |
| | | CCTTGGGTTCTGCTgACATT (SEQ ID NO: 416) | | | | |
| | | AAUGUCAGCAGAACCCAAGG (SEQ ID NO: 417) | | | | |
| EMR2-59 | 146 | CGTGCCAGTGCCTGCCTGGC (SEQ ID NO: 418) | 6 | CAG | TGccAGT | TGCCaGT |
| | | GCCAGGCAGGCACTgGCACG (SEQ ID NO: 419) | | | | |
| | | CGUGCCAGUGCCUGCCUGGC (SEQ ID NO: 420) | | | | |
| EMR2-60 | 132 | GTAAAaGCTACGGCACCTGC (SEQ ID NO: 421) | 6 | AGC | AAAAGcT | aaaaGCT |
| | | GCAGGTGCCGTAGCETTTAC (SEQ ID NO: 422) | | | | |
| | | GUAAAaGCUACGGCACCUGC (SEQ ID NO: 423) | | | | |
| EMR2-61 | 527 | CCCACtACGATGTGCAGGTG (SEQ ID NO: 347) | 14 | TAC | cAcTAcG | CaCTaCG |
| | | CACCTGCACATCGTaGTGGG (SEQ ID NO: 348) | | | | |
| | | CCCACuACGAUGUGCAGGUG (SEQ ID NO: 349) | | | | |
| EMR2-62 | 708 | CTCTCtGGATTTTGAAAAAC (SEQ ID NO: 358) | 18 | TGG | cTcTGGA | CTCTGGa |
| | | GTTTTTCAAAATCCaGAGAG (SEQ ID NO: 359) | | | | |
| | | CUCUCuGGAUUUUGAAAAAC (SEQ ID NO: 360) | | | | |

In Table 11, the lower case nucleotide refers to the edited nucleotide.

A representative amino acid sequence of full length EMR2 is provided by UniProtKB/Swiss-Prot Accession No. Q9UHX3-1, shown below.

(SEQ ID NO: 424)
MGGRVFLVFLAFCVWLTLPGAETQDSRGCARWCPQDSSCVNATACR
CNPGFSSFSEIITTPMETCDDINECATLSKVSCGKFSDCWNTEGS
YDCVCSPGYEPVSGAKTFKNESENTCQDVDECQQNPRLCKSYGTC
VNTLGSYTCQCLPGFKLKPEDPKLCTDVNECTSGQNPCHSSTHCL
NNVGSYQCRCRPGWQPIPGSPNGPNNTVCEDVDECSSGQHQCDSS
TVCFNTVGSYSCRCRPGWKPRHGIPNNQKDTVCEDMTFSTWTPPP
GVHSQTLSRFFDKVQDLGRDYKPGLANNTIQSILQALDELLEAPG
DLETLPRLQQHCVASHLLDGLEDVLRGLSKNLSNGLLNFSYPAGT
ELSLEVQKQVDRSVTLRQNQAVMQLDWNQAQKSGDPGPSVVGLVS
IPGMGKLLAEAPLVLEPEKQMLLHETHQGLLQDGSPILLSDVISA
FLSNNDTQNLSSPVTFTFSHRSVIPRQKVLCVFWEHGQNGCGHWA
TTGCSTIGTRDTSTICRCTHLSSFAVLMAHYDVQEEDPVLTVITY
MGLSVSLLCLLLAALTFLLCKAIQNTSTSLHLQLSLCLFLAHLLE
LVAIDQTGHKVLCSIIAGTLHYLYLATLTWMLLEALYLFLTARNL
TVVNYSSINRFMKKLMFPVGYGVPAVTVAISAASRPHLYGTPSRC
WLQPEKGFIWGFLGPVCAIFSVNLVLFLVTLWILKNRLSSLNSEV
STLRNTRMLAFKATAQLFILGCTWCLGILQVGPAARVMAYLFTII
NSLQGVFIFLVYCLLSQQVREQYGKWSKGIRKLKTESEMHTLSSS
AKADTSKPSTVN

A representative amino acid sequence of ADGRE2 which encodes EMR2 is provided by NCBI Reference Sequence No. NG_047146.1.

TACTTACTTATTTTCTGAGACAGAGTCTCACTCTGTGGCCCAGGCTGGAGTACAGTGGCGAGAGCTCACT
GCAACCTTGAACTCCTGGGCTCAAGAGATCCTCCCACCTCAGCCCCGTGAGTAGCTGGGATTCCAGGCAC
ATGCCATCACTCCTGGCTAATTTTTGCATTTTTTGGTAGAGATGGAGTTTTACCATGTTGCCCAGGCTGG
TCTCGATCCCCTGGGCTCAAGGGATCCTCCCGCCTCGGCCTCCAAAAGTGCTGAGATCACAGGCATGAGC
CACTCTGCCTGGCCGATTTTATCTTCCTTGATACAAACTCTTTTCAGAATTCTCTTTGTTTTTGCTTTGG
TCCCTGATAAAATCACATGTGAATATAACCGCAAAACACCACATGCCTGCATTACATGGCGTTACGTTAA
ACAACCATCATTTTGGCTGGGCGCAGTGACTCATGCCCGTCTCTACTAAAAATACAAAAAAATTAGCCTG
GCGTGGTGGTGTGCACCTGTAATCCCAGCCACTTGGGAGGCTGAGGCAGGAGAATGGCTTGAACGCGGGG
GGCGGAGGTTGCAGTGAGCCGAGATTGTGCCACTGCACTCCTGCCTGGGCGACAGAGCAAGAGACTCTGT
CTCAAGAAAACAAAACAAAACAGCCATCATTTTAGGAAACATTCATATGTGGAAACTACTATGTGTATTG
TTCACAATTTGCGGGAATAAAGAACTTTGCTTTGCAAAAAATATGTAAATTAAAAAATAAAAGGGAAGGA
AGGCATACCCATGAGCCTCCCTCATCACCAAGAACTGAGACATGACCTTGTTCCCATGCTCCCATCTTTA
TCTTGCTGATTACCACCACTCTGAATATTCTTTGCACCTTATTCCTTTGCTCAGTTACTTATTTTTTTAT
TTTATTTATTTTTGAGACAGGGTCTTGCTCTGTCACCCAGGCTGGAGTGCAGTGGTATGATCACAGCTCA
CTGCCACCTCGAATTCCTGGGCTCAAGCCATCCTCCCACTTCAGCCTCCTGAGTAGCTGGGACCACAGGT
GCGAGTCACCACACCTGGCGAATTTTTTATTTTTATTGTAGAGATGGAGGTCTCATTATGTTGCCCAGGA
TGGTCTCGAACTCCTGGGCTCAAGCAATCCTCCCGTCTCAGCATCCCCAAGTAGCTGGGATTACGAGCAT
GAACCACTGCGCCCGGCCCAGCCTTGTTTATTTTTATTTATTTATTTATATTTATGTATTTTTATTTTAT
TTTATTTTATTTAGACGGAGTCTCACTCTTGTCTCCCAGGCTGGAGTGCAGTGGCGCAATCTCAGCTCAC
TGCAGCCTCTGCCTCCCGGGTTCAAGTGATTCTCTTGCTTCAGCCTCCCAAGTAGCTGGGATTACAGGCA
CCCACCACCATACCCGGCTAATTTTTGTACTTTTAGTACAGACGGGGTTTCACCATGTTGGCCAGGCTGG
TCTTGAACTCCTGACCTCAGGTGATCTGCCCACCTCGGCCTCTCTGGGATTACAGGCATGAGCCACCGCG
CCCGGCAGCCTTGATTATTTTTTAAAGTCTTCTTGCATATGTTTGTATCCTGAAACCAAATCTTTTATTA
TCTTTGTATCTCAGCTTTTTTGCAAGGTGGCTTTTTCCTATGGGGCTTTGGAGACTTTTTCAAGATTCTA
GTATGGAATCTGTCATGCTGCAATTTAAGATTTTTTTCTCCTGACTTCACTCAGTTTTTAAATTTAAAAA
AGGAGTGTGGCTAGGCATGGTGGCATGCACCTGTAACCCCAGCATTTTGGGAGGCTGAGGTGGGCGGATC
ACTTGAGGGTCAGGAGATCGAGACCAGCCTGGCCAACATGGTGAAACCCCGTCTCTACTAAAAAATTAGC
TGGGCGTGGTGGTGTGTGCCTGTAATCCCAGCTTCTAGGGAGGCTGAGGCAGGAGAATTGCTTGAAGCTG
GGAGGCGGAGGTTGCAGTGAGCCGAGATCTCGCCACTGCACTCTAGCCTGGGTAACAGGGCAAGATTCCG
TCCCCCTCACACACACACAAAAAAAGTGAAAAGTTAGCCAGGCATGGCAACATATGCCTGTAGTCCCAGC
TACTTGGTAGGCTGAGGCAGGAGGATCACATAAGCCCAGGACGTGCAGGTTGCCTGAAGCCATAATTGTG
CCACCGCCACTGTGCTCCAGCGTGGGCAACAGAGACCCTGTCTCTAAAATAATAACAATAAATTATACTA
ATATAATTAATAATACCATTATTATTGTTGGTTGGATAATCTTGGGTTGCTTCTTTTTTTTTTTTTTTTT
TTTTTGAAACGGAGTCTGGCTCTATCGCCCAGGCTGGAGTGCAATAACGCGATCTCAGCTCACTGCAACC
TACATCTCCTGGGCTCAAGCAATTCTCCTGCTTCAGCCTCCCAAGCAGCTGGGATTACAGGTAATCTTGG
GTTACTTCTTAGCTGAGCAACCTGGGGCCAGGGGCCCCAAACCCCCAGGTTCCAGTTCCCCTTTTGACAA
ATGAGGACCATGTTCGCACCCCCCATCATTGCAGAGGTTGTCCAGAAGCTGCAAAATAGGTCAGCAGGAC
TGAGACTTCTGGACAAAGAGAAGGCGGAGAAACATAGGCAGACACCCCGGCAGGGACCAGATCAGCGAGG
CTGAAGGAGGCTTCCTTTAGCTGGGCGGCAGCGGAGTGGAGTGGCAGGAGTACTACTAATTTGCATGGTT
TACCATATAGGGCCCCACTCAGCACAGGCTCTGTGCTGGTGCTCTGCAGAGAAAAAGACAAAAGTCACTC
AGGGGGCTGACAGCCTAATGCGGCTCGAGGGCAGATGGTTGCTAGGGAAGAAGGGAGATGGTGAGAAGTA
TTGGAGAGACTCAAGAAAGCCGCCAGGAATGGTGACTCACGCCTGTAATCCCAACGCTTTGGGAGGCTGA
GGCGCGTGGATCATTTGAGGACAGGAGTTCGAGACCAGCCTGGCCAACATGGTGAAACCCTGTCTCCACT
AAAAATACAAAAATTATCTAGATGCGATGGCAGGTGCCTATAATCCCAGCTACTCGGGAGGCTGAGGCAG
GAGAATTGCTTGAACCCGGGAGGCAGAGATTGCAGTGAGACGAGATAGCACCACTGCACTCCGGCTTGGG
TGACAGAGCAAGACTTCGTCTCAGAAAAAAGTAATAATAAATAATAATAATAATAATAAAATAAAAATTT
AAAAAGAATGTTTCAGGCCGGGCATTGTGGTTCACGCCTGTAATCCCAGCACTTTTGGGATGCCAAGGCA
GGCAGATCACTTGAGGCCAGAAGTTCGAGACCAGCCTGACCAACATAGCGAAACTCCATCTCTGCTAGAA
ATACAAAAATTAGCCAGGCGTGGTGGCTTGCACCTGTAATTCCAGATACTTGGGAGGCTGAGGCAGGAGA
ATCTCTTGAACCCAGGAGGCAGAGGTTGCTGTGAGCTGAGATCACATCACCACACTCCAGCCTGGGTGAC
AGAGCGAGACTCTGTCTCAAAATAAATAAATGAATGAACAAATAAATAAAATAAAATAAAAATACAAAAA
TTAGCCAGGTGTGGTGGTGGGCACCTGTAGTCCGAGCTACTCTGGAGGCTGAGGCAGGAGAATCGCTTGA
ACCCGGGAGGCGGAGGTTGCAGTGAGTCGAGATGGCGCCACTGTACTCCAACCTGGGCAACACCAGAGTC
TGTCTCTAAAATACAGTAAAATAAAATTAAATTAAGAAGAGTCGATGATCACCATGAAGGGGTGTAGGGA
CAATCCCTGGCATCAGGAAGTGCCACTACCTCCCTGAGATTCTGCAGACGCCAGCTGTCTGACCTGCCCG
TCTGATCTGCAGAGAAGGGACGTTGAGCCCTGGGGTTCTAGCTGGAGTTTCTGGCTTTCTGAGAAAAAAA
AGGCAAAATCCCTCCAGACCTGGTCTGGCCCTCTCTCCTTCCAGCTTCTGCTGGTGTTCACTCCCCTCCC
CCACAGCCCAGGCCACCCCCACAGGAGCCCGGGACAGGAAGGGGTAAATTCTAGGACTGTGGATGTTTGA
TTATTCCCAGGACAATAGTTCTCTGACTTCAGTCATTAGTGACTTTTTTCTCCATCGCTGCAATCTCCCC
CTTTGAACAACTGAACTATATTTTCCTTAGCATTCATCACTTCTGCATCTTGAATTTACTCATGTAACAA
GGAGACTTGATTTCGGCATCCTCACTGAAAAACCCTCCTCATTGCCCCAGATAGTAGGGAAACCTAAAAA
TAAATATAAATATGAGGAACACAAAACCAAGTCCTTGAATTCTGGCTGGATACAGACACCTGCTAAGCCT
CAGGGTCACGGAGGCGCTAAAGACATGTTAGTGCCCAGTGGAGACTTTCCCCTGGGCTCATCGCAGGGAC
TCCAGGGCTGGGCTGCCGGCCTGTGGATCTGAGTCTCAGCTGGGTGAGTCACTTGTGCCTCAGTTTCCTC
CTCTGTCAAATGGGAATGACAACATCATTGCAGATGTTTTAAGGAGTAAATGAGTTAATATAGATTAAAA
AAAAAAAGCACTTAGGACCAGACCTGGTACAAATTAAGTGCTCCATAAATAATAGCATTTTCTTGGCAGG
ATAGCGAGATCTCGTCTCTACAAAAAAAAAAAACACTTAAGGAGAAAACTAGCCAGACGTGATGGTGCAC
AGCTGTAGTCCCAGCTCCTCAGGAGGCTGAGGCAGGAGGATCCCTTGAGCCCTGAAGTTTGAGGCTGCAG
TGAGCTGTGATGGTGACCCTGCACTCCAGCCTGGGTGACAGAGTAAGATCTCTTAAAAATATATATAGCG
TTTTAAAAAATGATTACGTACCTCAAGAGACAAGCTAATCATATGATTTATCCAGAAAGGAAAGAACCCT
CCTATCACATGATTCTCTGTTGATTAACGAAGTGCCCAGGGGCCTCTGAAATATAAGTTTCCCACAGGAG
GAAACTGGCTTAGAAAGACCAGAAAGATCCGGGGGGTCTGCTCTGTGCGGTGAAGCTTCTCTTCTTGGCA
CCTGCCTGGCATCGGAAGAGGGCCCCTTCTCCCTCCCTGGGCTTTTATGTGGACACTGTAATGCCTCAGT
TTTCTTTCTTTCTTTTTGTTTTTGACACAGGGTCTCACTCTATCACCCAAGCTGGAGTGCAGTGGCACCA
TCTTAGCTCACCGCAGCCTCAAACTCCCCAGCTCGGGTGATTCTTCCTGCCTCAGCCTCCTGAGTAGTAG
CAGCTGGGACCACAGACGTCTGCCACCAAGCCCAGGTAATTTTTTTTCTTTTTGAAGAAACAGAGCTTTG
CCATGTTGCCCAGGCTGGTCTCAAACTCCTGAGCTCAAGCGATCCTCCTGCCTCGGCCTCCCAGAGTGCT
GGGATCACAGGTGTGGAGAAATAGGAACGCTTTTACACTGTTGGTGGGAATGTAAATTAGTTCAACCATT
GTGGAAGATAGTGTGGTGATTTCTCAAGGATCCAGAACTAGAAATACCATTTGACTCAGCGATCCCATTG
CTGGGTATATACCCAAAGGATTATAAATCATGCTACTATAAAGACATATGCACATGTATGTTTATTGCGG
TACTATTCACAATAGCAAAGACCTGGAATCAACCCAAATGTCCATCAATGATAGACTGGATTAAGAAAGT
GTGGCACATATACACCAGGGAATACTATGCAGCTATAAAAAAGGATGAGTTCATGTCCTTTGCAGGGACA
CAGATGAAGCTGAAAACCATCATTCTCAGCAAACTAACACAGAAACAGAAAACCAAACACCACATGTTCT
CACTCATAGGTGGGAACTGAGCAATGAGAACACCTGGACACAGGGCGGGAAACATCACACACTGGGGCCT
GTCGGGGGGTGGGGGCCTGGGGGAGGGATTGCATTAGGAGAAATACCTAATGTAAATGACGAGTTGATAG
GTGCAGCAAGCCAACATGGCACATGTATGCCTATGTATCAAACCTGCTCGTTGTGCACATGTACCCTAGA
ACTTAAAGTCTAAAAAATTTAATTAATTAATTAATTAATTAATTTAAAAAGAAAGAAAGAAACCGAGCTT
CGCCATGTTGCCCAGGCTGGTCTCAAACTCCTGAGCTCAAGCGATCCTCCTGCCTCGCCCTCCCAGAGTG
CTGGGATCACAGGCGTGAGCCTCTGTACCTGGCCTCAGTTTCCTCATCTGGAAAACAGCAGGGGAAGATA
CTGCCAGCTGGGACGCAGGCTCTGCCAACTCTGGCATGCCAAAGCTCTGGGCACAGGGTGAGCCCTACCA
AAGTGGGAGCCCAGAGCTATTTCTGACAAGGCCCCTGCGTGCCTGCCCAGGGAAGAGCCAAGTGGCCCCA
GGCGAGACCGCCCCGCCCGGCTGCCACACCTTCCCCTGCACAGCAGCCAGCCTGGAACACACAGAGGCCA
GAGGGTTGGTCAGAGCCACATGGTGGAAACTCCAGCAGAGGGATTTTTAAAGCAGGTTGCACTTCATTCT
TGCTGAGCGAGTACACGTGTGTGTGTGTGTGTGTGTGTGTGTGTGTGTGTGTGTGCAGCGCCCCTGGTTC
TGTGTTTTTATTGTGCTTTCCTGCTGGCTTCCAGCTGCACCGCCAGTTCCGGGGAGGGCCCTGGGCCAGC
GGCTGTCCGCCCCCCCTCCTTTATAAAGTCCTGGCCTCGGGACAGCCCGCACAGCTGCCCAGCCTGCGGA
GACGGGACAGCCCTGTCCCACTCACTCTTTCCCCTGCTGCTCCTGCCGGCAGCTCAGCTGGAACCATGGG
AGGCCGCGTCTTTCTCGTCTTTCTCGGTAAGTACTTTGGGGCCCCGCTGGGGGTGGTAGCGAGGAAGCTC
CAGCGGGACCCCTTGGATGCGTCTGAGAAACGGGAGGCGCCGCTGGTGTCTGTGGGGCCGGCGATGGAGA
AAGCCACATGGTCAAGCAGGCACTCACGGGCACACGGCGAGAGGGGCTGGTGGTGCTTGGGCACCCCCAC
ACCCCGGCAGGAACTCCAGGGTGCTCTCGGAGCCTGCTACCCTGCCCGGAGGGATCCTCAAGCTTTCTAC
TGGAGCCAGTTTGGCATCTGCTCCATAAGAGGGGAAACTGAGGCACGGGGGAAGGAAAGGGGCAAAAAGT
GAGTGCTAGGACCCCAGAGCCACTGCTCTTAATATAGCGCATAGAAAGACATGGGCGGGGGGCGGGGAAA
ATAGAGGCCAGGACTTCTCCACTGCAGGGTACCCCCAGCTTCTCCCTTCTTTCTCTCTCTCTCTCTGTCT
TTCTCCTTCTGTCTCTCTTTCAGTCTCTCTATCTCCCTAATCTCCGTTTTCTCTCTACCTCTGCCTCTTT
GTCTCTCTCTCTGGTGAACAACAAGGAGATCCCTAGGTCCCCATCTCTAAAGCCCCTGGTGACCTGAGGA
GGGTCCCCCAGGACATGCCTTGGAAAGGGGAACTTCCTGAGTGCTGGGAACCGCAGCGCCACATGCACCC
CAACAGGCGGCCACAGCGTTTTCCTGTGGGAGGGGCGCCCGGGGTGAAATATCACCACTGGGTTTCAGGA
TGGGCCGCCAGGGCTGGCGGCGGTTGTGGTGTGTGTGCACCTGTGTCCCTGAGGAAGCATGCGGTTCCAG
GGCATCTTAGAGCCTGGGGGATCCAGCGCCATCATTTTTTTTTTTTTTTTTCTGAGGCGGAGCCTTGCTC
TGTCGCCCAGGCTGGAGTGCAGTGGCACGATCTCAGCTTGCTGCAACCTCCGCCTCCTGGGTTCAAGCGA
TTCTCCTGCCTCAATCTCCCAAGTAGTTGGGTAACAGGAGTCTGCCACCACGCCCAGCTAATGTTTTGTA
TTTTTAGTAGAGACGGGGTTTCACCATATTGGGCAGGCTGGTCTCGAACTCCCGACCTCAGATGATCTGC
CCACCTTGGCCTCCCAAAGTGCTGGGATTACAGTCCCGAGCCACCGTGCCTGACCATGCCTTCATTTTTA
TCTAAAGAAATGAAGATGGCAGTAAATGCTCAGGCACACCAGACAGCCCCCAAGTCAGAAGAGCGGCAGC
TGGAGCTGAGACCCCCACCAGGCTCATGGCCCTTTCCTACTCCTCAGTTCCTTAAACCCCACCCCCAAGC
CAAGCTAGGGAGGCTGAGGCAGGAGGATCGCTTGAGGCCAGGAGTTCAAGATCAGCCTGGGCAACAGAGC
AAGACTCTGTCTCTAAAATAACTTTTTAAATTATTTTTAATTTTTTTGGCCAGGCGTGATGGCTCATGCC
TGTAATCCCAGCACTTTGAGAGGCCAAGGCGGGCAGATCACCTGAGGTTAGGAGTTCAAGACCAGCCTGG
CCAACATGGTGAAACCCCATCTCTACTTAAGAAATACAAAAATTAGCTGGGCATGGTGGGTCATGCCTGT
AGTCCCAGCCACTCAAGAGGCTGAGGCAGGAGAACCGCTTGAACCCAGGAGGTGGAGATTGCAGTGAGCC
AAGATTGCACCACTGCATTCCAGCCTGGGCAACAAAGGGAGACTCCATCTCAAAAAAAAATAAATAATAA
AAATAAATAAATCATTTTAAAATTTTTGAGCTGGGAGCCGTGGCTCACACCTGTAATCCTAGCACTTTGG
GAGGCCAAGGCCGGCAGATCACCTGAGGTCAGGAGTTCGAGACCAGCCTGGCCAATATGGTGAAACCCCA
TCTCACTAAAAATACAAAAAATTAGCCAGGCACCATGGTGGGTGCCTGTAGTCCCAGCTATTTGGGAGGT
TGAGGCAGGAGAATTGCTTGAACCCAGGAGGTAGAGATTGCAATTAGCCGAGATCGCACCATTGCACTTC
AGCCTGGGCGACAGAGCAAGACCCTGTCTCAAAAAGAAAGAAAGAAAATTTAAAATATGGCGGTACTCAC
CTGTGGTCCCAGCTACTCGGGAGGGTGAGGCAGGGGGATCGCTTGAGCCTAGGAATTGGAGGCTGCAGTG
AGCTATAATCGCATCACTGCACCCCAGCCTGGGTGACCCAGAGAGACCCTGTGTCAAACAAAACAAAACA
AAACCCACCCCCATGAGGACAGGGTGACAGACTCTCTGGCTTTGAGCATCCGAGTGGGACGTGACATCTG
CTCACTCTGTTTGAACGATGGGCAGGATCTGAGCCTGAGAGGGGTCAGCATCCTCCTCCCACAAAGTGCA
TCACCCTTACGCCTCCTTTCCCACCCTGGGATCCCCTCTGACCCCCTTTCCTTTCTCTGTTGCAGCATTC
TGTGTCTGGCTGACTCTGCCGGGAGCTGAAACCCAGGACTCCAGGGGTGAGTCTGCTGGGAAGCAGAAAG
CACAGTCCACAGCCAGAGCCTGGGGAGGGTCCTGGACCCCCGCCCAGCCCCCTTCAGCCCAGGGAAAGAG
AGGGCTCGTGCACGGGAAACTCAGCGCTCTGCCCCATCTCCCCCAGTGCCCCCTTTTTGTGTATTCCCTT
ACCCCTCACCTTCTGACCGTGCTCCCTGCTCTTGCAGGCTGTGCCCGGTGGTGCCCTCAGGACTCCTCGT
GTGTCAATGCCACCGCCTGTCGCTGCAATCCAGGGTTCAGCTCTTTTTCTGAGATCATCACCACCCCCAT
GGAGACTTGTGACGGTACAGAGGCTTGAGGGCAGCGCAGGGGACATCTGCGATTATGAGGCATAGCCCAG
TGCCAGTGGGGGACAGAGGTTGTTGTGAGGGGCCACAGCCTTACCTTCCAGACTATCATCATGGCCAGAG
AAAAGAGAAAGAGGGCAGGTGTGGTGGCTCCCAGCACTTTGGGAGGCAGAGGCGGGTGGATCATTTGAGG
CCAGGAATTCAAGACCAGCCTGGGCAACATGGTGAGACCCTGTCTCTACAAAAAATACACAAAATAGCCG
CGCATGGTGGCACACATCTGGAGTCCCAGCTACTCAGAAGACTGAGGCGGGAGGATTGTTTGAGCCCAGG
AGTTTGAGGTTGCAGTGAGCTATGATTGCACCATTGCACTCTAGCCTGGGCAACAGAGTGAGATCCTGTC
TCAGGAAAAAGAAAGAAACAAAGAAAGAGGCCAGGTGTGGTGGCTCATGCCTGTAATCCCAGCACTTTGG
GAGGGATTGTCTGGGCAACAGGAGCAAGACCCCATCTCAAAAAAAAAAAAAAAAGACAGAAAGAGAGAAG
GAGAGAAAGAAAAGAGAAGAAAAGAAAAAGAGAGAGAGGGAGGGAGGGAAAGAAAAAGGAAAGGAGGGAG
GGAGGGAGGGAGGAAAGAAGGAAGGAAGGAAGGGAGGAAAAAGGAAAAAATATGAAGGGGGAGAAGTAAG
AAGTGAATAGGCATGGCTTCCTGGAGAGAGAGAAGCTGCGTGCTCAGGAATCTGGAGTCTGTGCCTCAGT
TTACCGTTAAGACTGGGAAGGGGGTACATTCTGGCCATAGGTTTTTTTTTCTCCCTTATTTTTCTTTTTT
CTTTTTTTTTTTGTTTTTTTTTTTTTTTTTTGAGATGGAGTCTCACTCTGTCGCTCAGGCTGAGGAGTGC
AGTGGCGCGATCTCGGCTCACCGCAACCTCCACCTCCCAGGTTCAAGCGATTCTTCTGCCTCAGCCTCCC
GAGTAGCTGGGATTACAGGCATGCACCACCATGCCCGGCTAGTTTTTGTATTTTTAGTAGAGATGGGGTT
TTGCCCTGTTGGCAGGCTGGTCTCGAACTCCTGACCTCAAGTGATCTGCCGGCCTCGGCCTCCCAAAGTG
CTGGGATTACAGGTATGAGCCACTGCACCCGGCTGCCATGGGGTTTAAATTTCTACAAAAAGACTGGGGA
TGGGGATATGGCGCTTCCTGTCCATCCCCCACCTCAGCACTGTCCACTGTGTCCAAATCTAGGATGCCAG
CCAGAGAACTGAGATCAAGTGTGGTCTTCAGGAATGGCCTGCTCCAGCTGCAGCATAGCCTGTGCAGTGC
AACTTGAGATCCTTTCCAAAAGATACTAAGCAGCAGGCCCCATGTTGGGGAGTCAGGACGGGGGTCTCTG
CCCTGACGGAACTCACATCTTTGGGAGATGACCTCCTGTCCTGTTGTGTTCCAGACATCAACGAGTGTGC
AACACTGTCGAAAGTGTCATGCGGAAAATTCTCGGACTGCTGGAACACAGAGGGGAGCTACGACTGCGTG
TGCAGCCCAGGATATGAGCCTGTTTCTGGGGCAAAAACATTCAAGAATGAGAGCGAGAACACGTGTCAAG
GTAAGAACCACCCCACATCCTCCATCACCACTGTCCATGAGGTTTGGGGTCACCAGAGCTGTTTCTGCAG
CATCCAGGGAGCAGGTACCCAAGTATAGGTTCAGTTCCTGGAGTCTGAGATGAGACAGGTGTGCATGTAC
CTGTTTCGCCAGCCCAGAGAGGCAGCGTGGTGTGAAGGCCAAGGGAAAACTCTGGGACCAGCTACCTGGG
CCCAAATCCTGGCTTTGCCACCTGTTACCTAAATGATATTACCAACATCACTACTGCTCTTAGAGCCTTG
GTTTACCCATCTATAAAATGAGGATGATAATAATGGCCTACTCCTATGCCTTCTAGGAGGACCAAGGAAA
GAATAGGTGCTTCTAACAGAGCCCAGCACACAGCAGGTGCTACAGGCATCTTCATTCTTTAAACGTTATT
ATGCACTGTAATCCCAGCACTTTGGGAGGCCGAGGCGGATGGATCACGGGGTCAGGAGATCGAGACTATC
CTGGCCAACATGGTGAAACCCTGTCTCTACTAAAAGTACGAAAATTAGCCGGGTGCAGTGGTGGGTGCCT
GTAATCCCAGCTACTCAGGAGGCTGAGGCAGGAGAATCACTTGAACCAGGGAGTCGGAGGTTTCAGTGAG
CGGAGATCGTGCCACTGCACTCCAGCCTGGTGACAGTGCAAGACTCCATCTAAAATAAATAAATAAATAA
ATAAATAAATAAATAAATAAGCAATGGATGCCTTTTAGGAGTTGTTCAGACAAGCTGGGCTCCTGTGGGG
CCACACTCCTGGCTGGCACTCAGAAGAAATCTATTTCGACACAGCCTGCCTCATCCCTTGCCATGGTCTT
ATTCCACACTGCAGCTCCAGCCCATGAGAAGCCAGGCCCCCATGCATCACTCTGCTTTGCCTTCTTTCCA
GTGGGAACTAGTTGGGTGCGGGAGGGAGCTTCAGACATCTGCAAGGCCAGATGGGAACCAGTAGCAGGTG
GACAGAGAAGGGAGATTAACACAGAGTACACAGAGTGGGAAGGGAAGGTGAGCAAAGGCAGGAAATGAAG
AAGCTGAGGCTGAGCCAAGGGTCCCAGGGGACATTAAGGGGGTTCAGCAGACCTGCCCTGCTGTAAAGAG
AAAAGTGGCAGTGCTCCTGTTCCCAGCCCTGTCCCTCCTGTTCTCCCTGCAGTCAGTAGAGCCTCCCTGG
TGGGAGGAAAGTGAGGACAGAGCAGAGGTGTACGTTCTGAGAGCACGCAGCTGCATAGAGCCCAGCAGGG
GGGCTGGTTCACAGGGGTCCCCTGTGAACAGATGCTACATTGCATGCACATATCTTGACCAAAGGACAGA
GCCTTCCTGTGAATTAGAAAAAGGACTCTCCGGGCCAGGCACAGTGGCTCACGCCTGTAATCCCAGCACT
TTGGGAGGCTGAGGCGGGTGGATCACCTGAGGTCAGGAGTTCAAGACCAGCCTGACAAACATAGTGAAAC
CCCATCTCTACTAAAAAATACAAAAGTTAGCCAGGCATAGTGGTGAGCACCTGTAATCCCAGCTACTCAG
GAGGCTGAGGCAGGAGAATCACCTGAACCCAGAGGCAGAGGTTGCAGTGAGCCAGGATCTCAATATTGCA
CTCCAGCCTGGGTGACAGAGCAAGACTTTGTCTCAAAAAAAAAAAAAAAAAAAAAAAAAAAAGAAAAGAA
AGAAACAAAAAGAAAAAGAGAAACAGAAAAGCCTCTCCTCTCCAGGTAGACACAGCCCCATTTGGGGAAT
GGAGAGGTTTTCAGCCTCTGCTCACCGCCTTGGCTGGGCATCCATATGTAGTCAGCAACCTACACAACTG
TACGTGGTGGTCATGGGTCCTTGTCAGTGAGAGCCTCTGTTTTAGTCTATGTTGTGTTGCTATAAAGGAA
TACATGAGGCTGGATCATGTATAAAGAAAAGAAGCTGATTTGGCTCATGGTTCTGCAGGCCATGTAAGAC
ACATGGCTCCAGCATCTGCTTCTGGTGAGGGCCTTAGGGAGCTTCCACTCTTGGCAGACGGGAAAGGGGA
AGCTGGCATCCCATGGCAAGAGGAAGGAAGCAAGAGAGAAGGGAGGAGATACCAGGCTTTTTCCAACAAG
CAGTTTTCATGGGAACTAACAAGGCGAGAACTCACTCATGACCCAAGGACAGCACCGAGATGTTCATGAG
GGATCTGCACTCAGGACCCAAACACCTCCCACGAGGCTCCATCTCCAACACTGGGGATCACATTTCAACG
TGAGATTTGGAGGGGACAAATATCCAAACTGTATCACCGCATGCACAGGTGGTGTTTGTAATTATTGAGG
AGGTGTGTTGCCATGTTAGAAACAGGCCAGCACTGGCCGGGCGCAGTGGCTCATGCCTGTAATCCTAGCA
CTTTGGGAGGCCGAGGTGGGCGGATCACAAGGTCAGGAGATCGAGACCATCCTGGCTAACATGGTGAAAC
CCCGTCTCTACTACAAATACAAAAAAATTAGCTGGGCGTGGTGGTGGGTGCCTGTAGTCCCAGCTAATCA
GAGACTGAGGCAGGAGAATGGCATGAACCTGGGAGGCGGAGCTTGCAGTAAGCCGAGATGGCACCACTGC
ACTCCAGCCTGGACGACAGAGCGAGACTCCGTCTCCAAAAAAAAGAAAAGAAAAGGAGAGAAGAGAAGAG
AGAAAAGAAAAGAGGCCAACACTGAGAGAGGGGGCTTAGTTGGTGGATAGGCAGAGACACAGATGAGCAG
GAATGAAGTGGGAGAGGGCTCTCATGGACCCCATCCGCACAGAACAGCCTGCTAGTTCGGAGCAGGACCT
GCCTCTTGAAGCCTCAGCTCAGGGAAGGGCTCCGTGGGGCCCTGTCTGCATCCCCCTCACCCCTCAGCAT
TCTTGTTTTTTTTGTCTACCTGGTGTCACAGGCCTCATAGGCAGGAGACCTGGGGCCACTCCCTCAGCTC
CTTGCCCCACTTACTCCAGGCATCACAGGGCCGTCCCAAACACCTGGTCACCTGTCGCTATCCAAACCTC
AAGGTTCAGAACCCTCCACAGAGGGTCACTTCCAAAAGCTCAGAACATCCCCCTGCCCATCTGGCATCTT
ATTTGTTTGTTTTGTTTTGTTTTATTAGTAAAGATGGGGGGGGGGTGTCTCACCATGTTGTCCAGGCTGG
TCTCGAACTCCTGGCCTCAAGTGATCCGCCCGTCTCAGCCTCCCAAAGTGCTGGGATTGCAGGCGTGAGC
CACCATGTCCGACCTGGTATCTTATTTGTACAAAACAAAAAGGGGGCTGGCACTCAGGATATTCGATGAG
CTCACTGCGTGGTGCATTCCAGCGCAGTATCCATTCTCTCTGCTATCATCAGCGACTGCCAGGATGAGAC
AGTCTCCACACAGCTCTGCTCACCTGGAACTCTCTCTGATCCCCCAGCTCATCATGGACCCAGGCACCCT
GTCCAGAAATGAAGGAGGAGGGAGATTTGAAGGCAGGGCCACTGTCCCCAGTCGAACCTGCCACTGGCCA
ACAACCCCACCTGGAGAAGATGCCTCGTGGAGCCCTGACCTCTTTTTCTGCCACTCAGTCTCTGTCCCTT
TCACTGAGCGAGACTGCCCTGACCCTTCCTGATAGTTTTGGTCCACCTGAGTGCACTGGAGGATGTTGTG
GCTAACGGTGGGCACAGTGACATGGACTCCCCATACTTGGAGAGTTGAGTGAATGGAAGAGGCACTAGTG
TGGACCCAATGGCAAGAGAGGTTAACATCAGGGCCATTGCTCCAAGCCTGATCCTCTCCCAGGAGGTCAA
CCCAGATCCCATGGGAGTGGAACCATTCAGAAATAGGTCTGTGTCCCTGGTGGAATGCCAAGAGATGAGA
TGACAGATACACCTCCATCTTTGCCCATCAGCATCTGTGCCCCTCTCCAGCCACTAAACCTTCACCCAGG
GCCAGTCTTTCTCAGCTATGGATTCTCACTACATCCTGCCATTTCCAAGACACCTTCCTGAGGATCAAAA
AAAGGCACCTTCACAGCCCTGGGGCAACCCAGCTGTATGTCGGTCCCCACTGTCCACCCTGGCCTGGGAC
CCACTGTCCAGGCAGCCCTGGCCTATGCAGCACCAGACTCATGCTCCCCTGGGGACTGGCTCAGGGGCCA
GTCTTGGTCCATGTGCCTGGAGCTGGGCTCTCAGAGCTAGTGTGGATGGGAGAGCACCATCCTGCTAGGG
CCACCGGGATTCTGATAACTCAGCCAGTGAGCAGCTGTCCTGGGGAAATCAAGGCCACCAGAAGCAGATG
CTGGAGCCATGCATCTTACACAGCCTGCAGAACCATGAGCCAAATAAACTTCTTTTCTTGCAACACAAAA
CGGACTAAACCAGAGGGTTCTCACTGAGAAGGACCCATGACCACCATGTACAGTTGCATAGGCTGCTGAC
TGCATATGGATGCCCAGCCAAGAGGGTGAGCAGAAGCTGAAAACCTGCCTACTCTCCATTCCTCAAATAT
GACTGTGACTACTTGAAGAGGAGATGCCTTTTTCCAATTCACAGGAAGGCTCTGTCCTTTGGCCCAGGAC
TGCCACTGGGAACCAAGACAGGTCCTACCTGGGCAAGCAGCATGCCCCCTGCACTTGGTCCCAAGAAAAT
CCCACCCTGACCTTTGTCCTGAATCCTCAGGCTGGGACATCCCTGCCCCAGCTGGGAGCTGCCCAGGGAG
GCAGGGGAGATGGGGAGGACTCAGATGCTTCCAGCCATATCTGAACAACAGAAGGCCTGGGCCACCATGC
TCACCATGGGACACCTCTGTGTGGATTAGAAAAGGGAGGAGATGGCCAGGCATGGTGGCTCATGCCTGCA
ATCCCAGCACTTTGGGAGGCCGAGGTGGGCTGATCACGAGGTCAGGAGATCAAGACCATCCTGGCTAACA
CGGTGAAACCCCGTCTAATACTAAAAATACAAAAAATTAGCCGGGCATGGTGGCGGGCGCCTGTAGTCCC
AGCTATTCGGGAGGCTGAGGCAGGAGAATGGCGTGAACCCGGGAGGCGGAGGTTGCAGTGAGTTGAGATC
AGGCAATTGCACTCCAGCCTGGGTGACAGAGCAAGACTCCGTCTCAAAAAAAAAAAAAAGAAAGAAAGAA
AAGAAAAGGGAGCAGACAGTGGTGCCTTCCAGATGCTTCCGTGCCAGGAAACATGGCGGACCCTCAGCCC
CGAAAGCTGCTCACAGCTACGGGCATTCTTCACCCTCTCCTTCCTCTTGCAGATGTGGACGAATGTCAGC
AGAACCCAAGGCTCTGTAAAAGCTACGGCACCTGCGTCAACACCCTCGGCAGCTACACGTGCCAGTGCCT
GCCTGGCTTCAAGCTCAAACCTGAGGACCCGAAGCTCTGCACAGGTAGAGGCCCCAGGAAGACGCTGTGA
GGCTGGACGGGAGCTGGGGATGGAGCTGAGTCAGGTCCTCCAAAGCAGCCGAGGAGGAGGGAGAAGATCC
GCAGGTTCCCACAAGGTCAAGGACCTGCTAAGCCCCTGCCTAAGGATTCACCTCCCAGGAAGGACTCGCC
ACACGGCAGGGAGGCGGCAGGGCCTTCAGGGCTTGGAGTGCCCTTGTGGGCCCCCAGACCTCACCCCTTC
CTCATCTGTCACGTGCGAGTGGAAGAAGGTGTCACTTCCAGTTCTAAGAAGGGGAACCTCCACCATAGTG
AGGGAGGGAGCAGGAGAGAGATTTTAGTCAGAAAGTCCAGCTCCCATGACTCAGTTTCCCTTTTGGTTTC
ACCTCCTATAAACTGGGGACCATGGTCCCTGCTGTGCCCATCTGGTAGGAAGACCATCATAAGATGCTTT
GAAGGTGAAGTTGAAGGTCACCAAGTGGTGGCACGTGCAATAATGGTCTTCAGTCTGAGGCAATGAAAAG
ATAGGGGAGTGGGTGCAGTGAGTGGAGGGCAGGCCCAGGCTGGTCAGGGACAGGGTTTGACCCTCTGGCT
TTGTCCTCAGATGTGAATGAATGCACCTCCGGACAAAACCCATGCCACAGCTCCACCCACTGCCTCAACA
ACGTGGGCAGCTATCAGTGCCGCTGCCGCCCGGGCTGGCAACCGATTCCGGGGTCCCCCAATGGCCCAAA
CAATACCGTCTGTGAAGGTCGAGAGCTCAGATCCCACGTTCCCAGAGACCCACAAACATCTGATCACATG
TTCAACGGCGCCCACACAAACCAAGCAGAATGAGCGCTGGAGGCGCCCGACTGTGTCAGGCGTTCATTCT
TCTGAGGCTAGATGAGAAAAGAGCAAGGGTCCTGCGGAAGGAGCTGGGGTACTGAGGGGGGAGGCTCAGG
GGGACCCCAGGCAACAGCTGATGACTCACTGGGAGGAAGGCGTTTCACCATATTCATAACCTGCTCATCT
GCACGGGGCCCACCTGCTGTGCCCAGGCCTCTCCACGCTTCCATAACCCAGCGTCCACCTCTCCAAGGGG
GGCACTAATGCCGGGAGGAACGAGCTGGGGGCACAGACAGGAGACAGGACCCTCTCCAGGCTGGGACAGG
ACCTGACCCCCTTCTTCCTGTCCTCAGATGTGGACGAGTGCAGCTCCGGGCAGCATCAGTGTGACAGCTC
CACCGTCTGCTTCAACACCGTGGGTTCATACAGCTGCCGCTGCCGCCCAGGCTGGAAGCCCAGACACGGA
ATCCCGAATAACCAAAAGGACACTGTCTGTGAAGGTATGACCTGGCCCTAGAAGCTCCCCACCCCCAGCA
CACACACTGACACGCTCCCGCCTAATGAGCCGCTTGTCTTGTTCCCTACAGATATGACTTTCTCCACCTG
GACCCCGCCCCCTGGAGTCCACAGCCAGGTGAGTGGCCCCCACAGGGACGAGGCGGCGGGAACTCCATCC
ACACAGCACTGCATCCGTCTCCTTGTTCTAAACTTCCCACCCGCCGTCCAGGCTCTCTGACCCCCACATC
TCCTCTCTCTGCAGACGCTTTCCCGATTCTTCGACAAAGTCCAGGACCTGGGCAGAGACTACAAGCCAGG
CTTGGCCAATAACACCATCCAGGTAAGGACAGGACCCAGGGCAAGGGGGCGAGGCAGGAAGGTGGCTGCA
TCCCACAGAGGCCTGGGGCAGTTTGGGTCTGGGAGGGGACAGGACCCAATGCAGTGGGTGCTGGTCTGAC
TCCCAGCATCTTTCAGGGCTGGTGGAAGCTAATGGACACCCAAGTGCACTTAATATCTTTCCTCTTGCTT
TCCCTGGACTTTGGGTTTAGGTCAGCATTGATATTGCACAGAAGTGTTGGCCTCTGAGGGGCCATAACCA
GAGTCAAGGATCACCTAGGGAATCCCATCCAATGTCATTTTTTTTTTAGACGGAGTCTCACTCTGTCGCC
AGTCCAGAGTTTAGTGGCCCGATCTCAGCTCACTGCAGTCTCTGCCTCCCGGGTTCAAGCGATTCTCCTC
CCTCAGCCCCCTGAGTAGCTAGGATTACAGGCACACGCCACCACGCCCGGCTAATTTTTGTATTTTTAGT
AGAGACGGGGTTTCACCATGTTGGCCAGGATGGTCTCGATCTCCTGACCTTGTGACCCACCCTCATCGGC
CTCCCAAAGTGCTGGAATTAGAGGCGTGAGCCACCGCGCCCAGCCGCCAATGCCATCTTCATCCCCCAGA
TAGACAGTCTCTAGGATCTGTTCCCTGGGGCTGAGCGGTTGGAGTCTTCATGCGGGCCCTCTGGCCCATG
GCTCACTAGGTCTGTGTCCACATCCCTCCAGAGCATCTTACAGGCGCTGGATGAGCTGCTGGAGGCCCCT
GGGGACCTGGAGACCCTGCCCCGCTTACAGCAGCACTGTGTGGCCAGTCACCTGCTGGATGGCCTAGAGG
ATGTCCTCAGAGGCCTGAGCAAGAACCTTTCCAATGGGCTGTTGAACTTCAGTTATCCTGCAGGCACAGG
TAGGTCCCTGGGTCTGCCCCAGACTCCAGCTCTGCATGTTTTCTGTCTCCTTCCTTTCCCAGTCCCACCA
GAGCCAAGTGACCACACCTGTATATCAGTGTTACTCTCATCGACAAACTAAAATAAGAGATTAAAAAGAA
ATACATACCAGTCTGGACAAGATGGCAATACTCAATCTCTACAAAAAAAAAAAAATCTTTTAAATTAGAC
AGATGTGATGGCACAACTGTGGCCCCAGCTAGTTGGGAGGCTGAGGTGAGAGGATCGCTTGAGCCCAGGA
GGTTGAGTCTGCAGTGAACTATGATTGCATCACTGTACTCCAGCCTGGTTCACAGGGCAAGACCCCATCT
CAAAAAAGAGAAGACGAAGAGGAGGAGGAGGAAGAAGAAGAAGAAGAGAAGGAGGAGGAGGTGAAGAGGA
GGAGGAGGATAAAGAGGAAGAAAAAGGAGGAAGAGGGCAGGAGGGGGAGGACAGAAGGAGGAGGACAGGA
GGAGAGGAGGGGAGGAGGAAGAGGAGGAAAGAGGATGGGAGGAGGAGGACAAGAGGAAGAGGACGGGAGG
AGGAGGACAGGATGGGGAGGAGAGGAGGAGGAGAGAAGGAGGAGGAGAGGAGGAAGAGGAGGAATGGAGA
AAGAGAGGAGGAGGAGAAAAGGAGGAGGAGAGGAGGAGGAGGAGAGGAGAAGGAGGAGGAATGGAGGAGG
AGAGGAGGCAGAGGAAGATAGGAGGAGGAGAAGGAGAAGAGGAGGAGAAGAGAAGGAGGAGAGGAGGAGG
AGACGAGAAAGAGCGGAGGAGAGGAGGAGAGGAGGAGGAGCACAGGAGGAGGAGGAGAGGAGTAGAGGAA
GAAGGAAGAATAGAAAGAAGGAAAGGAAAGGAAAAAGAAATGCATAAATAACATGCATTTTCATTGTAAT
ATGAATTATCAGCAGTAAATAATTATGATGAAGAATTTAAAATAACAAAATAGAAATCACTTCAAAAGAG
AACAATTTATTTAATTTGGAATACTATAAATTAATATCATTTGATCTCTAAGTATGATTTCTTTCTTGTT
TGTTTTTGTTTGTTTTGTTTTGTTTTGTTTTGTTTTTTTGAGACAGAGTCTCACTCTGTCGCCCAGGCTG
GAGAGCAGTGACGTGATCTCGGCTCACTGCAAGCTCCGCCTCCTGGGTTGACAGCATTCTCCTGTCTCAG
CCTCCCGAGTAGCTGGGACTACAGGTGCCCGCCACCACGCCCAGCTAATTTTTTGTATTTTTAGTAGAGA
CGGGGTTGCACCGTGTTAGCCAGGATGGTCTCTCGATCTCCTGACCTTGTGCATAATCCACTTGCCTCGG
CTTCCCAAAGTGCTGGGATTACAGGCGTGAGCCACCACGCCCGGCCTGCATAATAATTTTTAAAGCATGA
AAATGCATATAGCATTAATTTTATTAAGATAAAATTCACATAACATCAAGTTCACCACTTTAAAGTGTCT
GTTTCACGTGGGGCGTGCCTGTAATGCCAGGCGCTCACACCTGTAATCCTAGCACTTTGGGAGGCTGAAA
TGGGTGGATCACTTGAGGTCAGGAGTTCAAATCCAGCCTGGCCAACATAGTGAAACCCCATCTCTACTGA
AAATACAAAAATTAGACAGGCGTGGTGGTGCACACCTGTAATCCTAGCTACTCAAGAGGCTGAGGCAGGA
GAATCACTTGAGCTTGGGAGGCAGAGGTTGCAGTGAGCCAAGTTTGCACCACTGCACTCCAGACTACGTG
AGACAGAGTGCCACTCCATCTCAAACAAAAAAATTAAAATTAAAAATAAAAATAAGAAAAAATTTTAAAA
GCGTGTGTTTCGGTAGAATCTAGTATATTCAGAAAGTTATACAACCGTCACCTCTATTTCTAAAATGCTT
GCATCAGCCCAAAAGTGAACACTGCACATGGTAAGCAACCACTCCTCATTCCTCCCTTCCTCCAACCCAT
GGCAGCCGCTAATCTGCTTTCTGTTTCCATGGATTTTCCTAATCAGGATATCTCACATAAATGGAATCAT
ACAATATGTGACCATCTAGTCTAGCTCCTTTCACCTAGCATAATGGTTTTGAGGTTCATGCATGCCATAG
CATTTATCAATATTTCATTCTTTTTCATGGCTGAATACTATTCTATTGTATGAATACATCACTTTTTTTT
TTTTTATCCATCCATCCATTGATGGACATTTGGGTTGTTTCCACCTTTTGGCTGTTGACAAAAAGAGTCA
AACTCTGTAAAATATTTGAAGAGATTTATTGTGAGCTAAATATGAATGTATGACTGACCATGGCCCGTGA
CACAGCCCTCAGGAGCCCCTGAGAACATATTCCCAAGTGAAGTGGTGTTGTTTTCTGGGGTAATACCTGA
GGTTCATTGCCTCATGCCAAGAAAATTAAGGACACAGACACACACCAGGAGTGAGTTTATGAGTGGAGGT
TTAATAGGCAAAAGAAAGAGAAAGGAGAACAACTCTCTCTCTTGTGAGAGAAAGGAATGCCCAAATGGGA
CTTCCCACCCATGCCAGAGGGCACCGGATTTTATAGACAGGCTTGAGGAGGCAGTGTCTAATTTACATAG
GGCCCAAAGATTGGTTGGAACAGGTGTGACGTTTACATAAGGTGCAGGGAAGCTGGCTGCCCCACCCTAA
TCTTATTATGCAAATGGAGTCTTCGCCAGCTCCTTACTGTATACGTGGTTGGCAAAGAGATGGGAAGATG
GAGCCGCCATTTTCAACATGCCTAGTCCCAGGTGGCCTTTTCCTATTGGCACAGCTGCCAGCATTCACCT
GTGCAGGCTTCCAGCTTGCTTGCCTATGTCTGCAGCTCGATTTTACAGGCTGCTCTTTGTTAGAAAAAAA
AAAAATGATATAGGGGCTGATTTTCATTAAAAGGAAAGACTTACCCAGGACTTCCTTACCCACACTCTCT
GCCTAAATAATTCCTTTTTAACTTCTATATCACAAGGTAGTCGTGGTACAGCTTGTTGTGATTGTTGTTG
TTGTTGTCTTCTGTTGGAGACAGAGTCTTGCTCTGTCAACCAGGCTGGAGTGCAGTGGTGCGATCCTGGC
TCACTGTAGACTCCACCTCCCGGGTTCAAGCGATTCTCCTGCCTCAGCCTCCCAAGTAGCTGGGACTACA
GGCGCCCGCCACCACGCCCGGCTAATTATTTTGTGTGTTTTCAGTAAAGACAGGGTTTCGCCATGTTAAC
CAGGATGGTCTTGATGTCCTGACCTCGTGATCCGCCTGCCTCGGCCTCCCAAAGTGCTGGGATTACAGGC
GTGAGACACCACGCCTGCCCCCGCAACTTGGTTTTATACATTTTAGGGGGACATGAGACATCAATCAAAT
ATGTTTAAGCTATACATTGGTTCAGTCCAGAAATGCAGGACAATTTGAAGTGGAGGTGTGGGGCGGGTGC
TTCCAGATTATAGGTAGATTTAAGCTTTTCTGATTGGCAGTTGGTTGAAGGAGTTATTATCCATAGAAAA
GAATGTCTGGAATACGATAAGGGGCTGTAAAGGCTGAAGTTGTATCATCCAGATGAACTCTTCAGGTATC
AGGCTTCAGAGAGAATAGATGGTAAATGTTCCTTATTAAGCTTCAGGTCTGTGTTATGTTAAATGCTGGT
CAGCTTTTCCTCAACTCCAAAAGGGAGGAGGGCATGACACATGTCAGACCCCCCACTTCCCACCATAGCC
TGAACTAGTCTTTCCAGTTAAATTTAGAGTGCCCTGGCCAAAAAGGAAGTCCATTCAGATGGTTTTGAGG
GGGGCGGGAGAGGACTTCGAATTTTTTTTTTTTGTTTAGATGGCTGGTGTGAATAGTGCTCTTATGAATA
TTAGTGGACACATTTTGGGTTGAATAACTGTCTTCAGTTCTTTGGGGTATATACTAAGGAGTGGAATTGC
TGGGTGATATGGTAATTCTATGTTCAGCTTTTGGAGGAACCACCAAACTTTTTCCAAAGCAGCTGCATCA
TTCTAATTCCCCACCAGAAACATACACGGGTTATCCTCTCCACACCTTCAGCAAAACTTGTTACTTCCGT
TTTCTTTTGTTCTGTTTTTAATTGCTTTTATTGATACATAAATCTTACATATTTATGGGTACATGTGATA
CTTTGTTGCTTGCCTAGAATGTGTAACGATCAAGTCAGGGTATCTCAGGTATCCACCACTTTGAGTACTT
ATCATTTGTATGTGTTGGGAACAATTGAAGTCCTCTTTTGTAACTACTTTGAAATACATCATACAGTCTT
GTTAATTATAGTCACTCTGCTCTGCTGTCAAACAATAGAACTTGGGCCGGGCACGGTGGCTCACGCCTGT
AATCCCAGCACTTTGGGAGGCCGAGGTGGGAGGATCACGAGGTCAGGAGATCGACACCATCCTGGCTAAC
AGGGTGAAACCCCATCTCTACTAAAAATACAAAAAATTAGCCAGGCGTGGTGGCGGGTGCCTGTAGTCCC
AGCTACTCGGGAGACTGAGGCAGGAGAATGGCGTGAACCTGGGAGGCGAAGCTTGCAGTGAGCCAAGATG
GTGCCACTGCACTCCAGCCTGGGTGACAGAGTGAGACTCCGTCTCAAAAAAAAAAAAAAAAAAAAATCTG
CTTAATAAGCGGAGAGGTGGCTGGCTTGGTGGCTCACACCCGTAATCCTAGCACTTTGGGAGGTCAAGGC
AGGCAGATCACCTGACATCAGGAGTTTGAGCCCAGCCTGGCCAATATGGTGAAACCCTGGCTATTAAAAA
TACAAAAATTAGCTGGGCTTAGTGACACGTGCCTGTAATCCCAGCTACCTGGGAAGCTGAGGCAGGAGAT
TCGCTGGAACCCAGGAGGCAGAGGCTGCAGTGAGCTGAGATCGTGCCACTGCACTCCAGCCTGGGTGACA
GAGTGAGACTCCGTCTAAAAAAAAAAAATATATATATATATATGTATAACTTATACTTTTTGTCTAACTG
TATGTCTCTACCTGTTAACTCACCTCTCTTCATCCCAGTGATACAGGAGTGAAAAAGAAATTATTTAGGC
AGTTAGCAAGGGTCAGAGAGTCCTCAGTAAGGTTTCCCTTTTAATAAAAACCAGCCCCCAAATCATTTCT
TTTCTAATAACGAGCAGCCTGAAAATTCGAGCTGCAGACATAGAAAAGCAAGCTGGAAGCTTGCACGGGT
GAATGCCGGCAGCTGTGCCAATAGGAAAAGGTTACCTGGGGGCCCGACATGTTCAACGTGGGGGCTCCCT
CTTCCTTTATCTTTGTCAACCACGTGTACAGTACAGAAGCAGGCAACGTGGAGCCAGCCAGGTAGAGAAT
CCATTTGCATAATAAAAGATTAGGCTGGGGCAGCCAGTTTCTTCATGCTGTATGTAAATGGCTCACCTGT
TCAGACCAATCTTTAAGCCTTATGTAAATCGGACACCGCCTACCCAAGCTCATCTATAAAACTTCGTGCA
TTTCACCACGCAACCGGAAGACCCACTTGGGAGCCCCTGTCTCTCTGCAGGAGAGGGAGTTATTCTCTTT
TTCCTTTTTTTTTTTTTGAGACGGAGTCGTGCTGTGTCCCGCAGACTGCAGTGCAGGGGCGCGATCTCGG
CTCACTGCAAGCTCCGCCTCCCGGGGTCACGCCATTCTCCTGCCTCAGCCTCCGGAGTAGCTGGGACTAC
AGGCGCCCACCACCATGCCCAGCTAATTTTTTTGTATTTTTAGTAGAGACGGGGTTTCACCGTGTTATCC
AGGTTGGTCTCGATCTCCTGACCTCGTGATCCTCCCGCCTCAGCCTCCCAAAGTGCTGGGATTACAGGCG
TGAGCCACTGCGCCCAGCCAGAGCTATTCTTTTTTTCTTTCGCCTATTAAATCTCCACTCTTAAACTAAC
TTCTTATGTGTCCACATCCTCAATTTCCCTGGTGTGAAACAGTGAAACTTGGGTATTTACACCACTTCAC
TAGTGAGATGATACCTCATTGTGGTTTTGATTTGCCCTTCCCTGATAACTAATGATGTTTAACATCTTTT
CATGTGTTTGCTCACCATTTGCATGTCATTTTTGGAGAAATGTCTATTCAAATCCTTTGCCTATTTTTAA
TTGTGCTGTTTGTCTTTTGGTTGTCCAGTTGTAAGCATTCTTTAAACATTCTGATAGTAGATCATCTGAG
ATGAATGATTTGCAAAATATTTTCTCCCGTTCTGTAGATTGTCTTTTCATTTTCTTGTCTTTTCACTTTA
ATGAATAGAAGTTTTAAATCTTGAAGACATTTATTTTATCTAATTTTTCTTTTGTTGCATGTGCAATTGG
TGTCATATTTAAGAATTCATTGACAGGGGGTGGTGGCTCACGCCTGTAATCCCAGCACTTTGGAAGGCCA
ATGTGGGCGGATCACCTGAGGTCAGGAGTTTGAGACCAGCCTGGCCAACAAGGCGAAACCCTGTCCTTAC
TAAAAATAAAAATAAAAATAATTAACCAGATGTGTTGGCACATGACTGTAATCCTAGCTACTCAGGAGGC
TGAGGCAGGAGAATTGCTTGAACCTGGGACACGGAGGTTGCAGTGAGCTGAGATTGTGCCACTACACTCC
AGCCTGGATGACAGAGCAAGACTCTGTCCCAAAAATAAAAATAAAAAAAGAATTCATTACCGCATCCAAG
ACCAGGAAGATTTACCCCTGTTTCCTCCTAAGTGTTCTAGTTTTAGCTTCTAAGCTTAGGTCACTGGCTT
ATTTTGAGTTAATTTTTGTTTGTTGTGTGAGGGTAGGGTTAAAACTTCCTTATTTTCATGTTACTATCCA
GTAATCCCAGCACTATTTGTTGAAGAGATTATTCTTTCCCCCATTGTATAGTCTTGGCACCCCTTGTTGA
GAATCAACTGACCATAAGATGGATGGGTTTATTCCTGGACTCTCAATTCTATTCTGTTGTGTCCTTATGC
CAGTATTACTGTCAGTTTGTAGTAAGTTTTGGAATCAGGTAGTGTGAATTCTCCAACCTTCTTGTTCTTC
TTCAAGATTGTTTTTGGGTATTTGGATTCCTTACAACACCATATGAATTTTAAGATGAACTTTTCTTATT
CTGCAAAGTAATAATAGGCTAAAATATTGATAGGGATTGCATTGAGTCTCTAGACCACTTTGGGGGAGTA
TTGTTATCTTAACAATAGAAAGTCTTCCAATCCATGAACATAGGCTGTCTTTCCATTTAGAGAGGGCCTT
TTATTTCTTTCAAACACTTTTGTAGGCCAGAAGCAGTGGCTCACGCCCAATATTGTCAGAGGCCACCAAG
GTGGGAGAATTGCTTGAGCCCAGGAGTTCAAGCCCAGCCTTGGAAATATACTGAAACATGATCTCCACAA
AAAATTTAAAAATTAGCTGGCTGTGGTAGTGAGCACCTGTAGTGCCAGCTACTTGGGAGGCTGAGGTGGA
AGGATTGGTTGAGCCCAGGAGACTGCAGTGAGCTATGATCATGCCACTGCACTCTAGCCTGACCAACAGG
TGAGAATTTGTCTCAAAAAAAAAAAAATACTCAAAAAACTTTCCTATAAGTCTTGAGTCTCCTTCGTAAA
ATGTATTCCTAACATTTTTGCTGAAAATTTTTGCATCTATGTCCATAAGAAATATTGGTCTATAATTTTA
TTTTCTTGTGATGTCTTCATCTGGTTTTGTTATCAGAGCAATATTAGACTCATAAAATTACTTAAATAGT
GTTTCCTCCTCTTCCATATTTTTAAAAAGGTAAAAGATTCATGTTCATTCTTTCTTAAGCTATTGGTAGA
ACTCACCAGTGAAGTCATCTGGTCTGGGCTGTTAGTTTTGAGAGGTTTTGATTACTGATTTAATCTGTTA
TAGGTCTGTTCAGATCTTCTATTTCTTCTTGAGTTGATTTCAGTGGTTTGTGTGTCACTAGGAATGTGTC
CATTTCATCTAGGTTACCTACTTTGTAGATATACAGTTGCTCACACTATTCTCTTCCGATATTTTGTAAC
TTCAGACTATATATGTATATAGTATATATATTAAGTCATAGTATATCAACCACTGCAGGCACGATTCCTA
ACCTAACCCCCCTTATGTAATTCCCACAACTCATAGAATTGTCCCTGGAGGTGCAGAAGCAAGTAGACAG
GAGTGTCACCTTGAGACAGAATCAGGCAGTGATGCAGCTCGACTGGAATCAGGCACAGAAATCTGGTGAC
CCAGGTAATGGCTGAGATGGGGAGGTGGTCAGTGAAGCTTCATGGTGGGAAAAGATGCCCAAGAATGTGA
AAAATAAGAAATTTAAAGATTAAAGGGAAGTGTGGGATGTTTGGAGTGGGGCTGTAAGAGGAGAGTCCAG
CTGAGGAGGAACGGACCTTGTTGGGGACCCTGAGATATTAACCGTGGCCTTGTCCTGCAGGATGTGATAC
ACATAGAACTAAAGATTGAAGGAAAGTGTGGGAGGGGTGAAGCCAAAGGAAGGTAGCCCATCTGAGTAGA
AAGTTCTGTGCAGGGCAGAGGAACTCTGGTGTGGACCCAGAGATTCAACCTACTCCAAGATTCAACCTTG
GCCTCGCCCTGCAGGCCCTTCTGTGGTGGGCCTTGTCTCCATTCCAGGGATGGGCAAGTTGCTGGCTGAG
GCCCCTCTGGTCCTGGAACCTGAGAAGCAGATGCTTCTGCATGAGACACACCAGGGCTTGCTGCAGGACG
GCTCCCCCATCCTGCTCTCAGATGTGATCTCTGCCTTTCTGAGCAACAACGACACCCAAAACCTCAGCTC
CCCAGTTACCTTCACCTTCTCCCACCGTGTGAGTGCTGGTGGAGTTGGTTGGTGGGTGAATAGTCTGAGT
CCGGGCATAGCCTTGCTGCTCAGCTCAGCCCTGGGGCTCAGGGGTCTCTGTTATGGGTACATTATCTCCC
CAGGAAAGTCAGTCCTTCCCAAGCCGGCTTTGGGTAAGCATTTCTGAGCATCCGACCCACCAGCTCACAC
CTGTGTTCTTTTTTTTATTGAGACAGAGTCTCGCTGTGTCAGCCAGGCTGGAGTGCAGTGGCACGATCTC
AGCTCACTGCAGTCTCCGCCTCCCAGGTTCGAGCGATTCTCCTGCCTCAGCCTCCCTAATAGCTGGGACT
ACAGGTGTGTGCTACCACGCCCAGCTAATTTTTGTATTTTTTTTTTTTTTTTTTTTTTTTAGTAGAGATG
GGGTTTCACCATGTTGGCCAGGCTGGTCTCAAACTCCTGACCTCAAGTGATCCACCTGCTTCAGCCTCCC
AAAGTGCTGGGATTACAGGCGTGAGCCACCATGCCCAGCCCACACCTGTGTTCTGTTCCTGCAGTCAGTG
ATCCCGAGACAGAAGGTGCTCTGTGTCTTCTGGGAGCATGGCCAGAATGGATGTGGTCACTGGGCCACCA
CAGGCTGCAGCACAATAGGCACCAGAGACACCAGCACCATCTGCCGTTGCACCCACCTGAGCAGCTTTGC
CGTCCTCATGGCCCACTACGATGTGCAGGTGAGACCCTTAGGAGGGGATGCACTCTGCATTTATTGCCGT
GTAACAAATCCCCAGAGACGTAGCAGCCTTTTAAAAAAATATATGCATTATCTCACAGCTCTGGGTCAGC
AAGCTGGCATAGCAAGATGGCTTTTCTGCTCAGGGTCTTACAAGACTGAAATCATCATGTCACCCCACCC
CCAGGGCTGCCATTTCATCTGAGACTTGAGGTCCTTCTCCAAGCTCCCTGGTTGCTGGCAGAATTCAGTT
TCTTGTGATTGTAGGACTGAAGTCTCACTTTCTTAGCTGTCAGGAAGGCATCACATTCAGTTCCTAGAGG
CCACTCATATCCCTTCTCACATGACCCTCTGGCAAACTTCTAGCATTCCTATCTGGAGGACAAGGAAAAA
AATTTCCTCACGCAGAATCCCTCTTAAGCTTTGTATTAGTCGAGGTTCTCTAGAGGGACAGAACTAATGG
AATAGATAGATAGATAGATAGATAGATAGATAGATAGATAGATAGATAGATAATTTCTGCCTGCTTATAT
TCTAGCCACGCTGGCAGCTGATTAGATGGTGCCCACCCAGATTAAGAGTGGATCTGCCTTTCCCAACCCA
CTGACTCAAATATATACATATATAGAGATATATAGAGAGAGATCTATATATATAGATAGATATAGATATA
TATAAAGGGGAGTTTATTAGGTATTAACTCACATGATCACAAGGTCCCACGATAGGCCGTCTGCAGGCTG
AGGAGCAAGGAGAGCCAGTCCGAGTCCCAGAACTGAAGAACTTGGAGTCTGATGTTCAAGGGCAGGAAGC
ATCCAGCACAGGAGAAAGATGTAGGCTGGGAGGCTAGGCCAGTCTCTCTTTTCACATTTTTCTGCCTGCT
TATATTCTAGCCACGCTGGCAGCTGATTAGACAGTGCCCACCCAGATTAAGAGTGGATCTGCCTTTCCCA
GTCCACTGACTCAAATGTTAATTTCCTTTGGCAACACCCTGATAGACACACCCAGGATCAATACTTTGTA
TCCTTCAATCCAATCAAGTTGACACCCAGTATTAACCGTCACAAGTTTTGAATCTGATTCTCCAGGAAGA
GCCCAGAGCTTATAAAGACTCATCTGATTAGGTCTGGCCCACCCAGGGTAAACTCTGCCTTTTTTTTTTT
TTTAGACAGGGTCTCTGTCACCCAGGCTGGAGTGCAGTGGCACAGTCATAGCTCACTGCAGCCTTGACCT
TCTGGGTTCAAGGGATCCTCCTGCCTCAGCCTCCTGAGTAGCTGGGACTACAGGCATGCACCACCATGCC
CGGCTAATTTTTGTATTTTTACTAGAGATGGGGTTTCACCAAGTTGGCCAGGCTGGTCTCGAACTCCTTA
CCTCAAGTAATCCACCCGCCTCGGCCTCCCAAAGTGCTGGTATTACAGGCAGGAACCACCACACCTCGCC
CACCTGACTTTCTTATCTCATCACTTAGAGTAGATCCAAGGGACATCATCATCATATCCTGCGGGAGAAA
GGGCCAAACCACCCTTTTGTTTTCCATGACAGGAACATCTTATCAATATCCTCCCAGGCAGCAAGCCATA
CCGCCCAGCCCCTCCCGCCCAGACCTGTAATTACCCCAGCCTGTAAGCGGCAGTGGGTTCTGGCACGAAG
CTAGCTCCCCCCTCCACAAGTCTCCTGCTGGACATAAACCTGCATTGCTGTAGAGCTGCCAACTCTCTGT
CTTTCTTTAACTCTCACTTTCCCTTCAAAACCTAACGGGCCCACACCTGGAATCTCTGTGCTTTGGGAGG
CTGAAACGGGAGGATCACTTGAGCCCAGGAATTCGAGACCAGCCTGTGCAACACAGAAAGACCCTGTCTC
TACAAAAATAAAAAAAAAATTAGGACGGGTGTGGTGGCTCACGACTGTAATCCCAGCACTTTGGGAGGCC
GAGGCGGGTGGATCATGAGGTCAGGAGTTCAAGACCAGCCTGGCCAACATGGTGTGACCCTGTCTCTACT
AAAAGTATAAAAATTAGCCTGGCATGGAGGCACGTGCCTGTAATCCCAACTACTTGGGAGGCTGAGACAC
GAGAATCGCTTGAACCGAGGAGACAGAGGCTGCAGTGAGCAGAGATCGCACCACTGCACTCCAGCCTGGG
CGACAGAGCAAGACTGTCAAAAAAAAAAATTAGCAGGGCATGGTGCCATACATGACTATAATGCACACCC
GTTGTCACCTACATGACTACGCCTGTATTCCTAGCCACCCATGAGGCTCGGGTGACACCTTCGCAAATTA
AATTAAATTGGTGACATCTTCACAAATTTACCTGGCTCCCTCATTCTTGAATGTGCTATGCCAAAGTAAA
AATTTTAAATGGTGGTGGGGTCATTACCCCAGAGCAGGAAAGCATCACTGTGAACTCTTCTTTTCCGGGT
CCCAGGAGGAGGATCCCGTGCTGACTGTCATCACCTACATGGGGCTGAGCGTCTCTCTGCTGTGCCTCCT
CCTGGCGGCCCTCACTTTTCTCCTGTGTAAAGCCATCCAGAACACCAGCACCTCACTGCATCTGCAGCTC
TCGCTCTGCCTCTTCCTGGCCCACCTCCTCTTCCTCGTGGCAATTGATCAAACCGGACACAAGGTATTGA
CAGCGGTGTTCCAGAGGGCTCCCTTCCTTGACGCAGGACACGCTGGCTCATGGAATGATGCGGCATTTGA
TTCCTTAATATAATAGCCCGGCTGGGCGTGGTGGCTCATGCCTGTAATCCCAGCACTTTGGGAGACTGAG
GTGGGCGGATCACCTCAGGTCGGGAGTTTGAGACCAGCCTGACCAACATGGAGAAACCCCGTCTCTACTA
AAAATACAAAATTAGCCAGGCATGGTGGCGCATACCTGTAATCCCAGTTACTCAGGAGGCTGAGGCAAGA
GAATCTCTTGAACCCAGGAGGTGGAGGTTGTGGTAAGCCGAGATCGCACCATTGCATGCCAGCCTGGGCA
ACAAGAGTGAAATTCCGTCTCAAAAAAAAAAAAAAAAAAATATATATATATATATATATGTGTGTGTGTG
TGTGTGTGTATACGTATATATACACGTATATATATATAGGCCCTTGAGTCCTGGGGAATATTAGAAGAGA
ACACCACCAATTTCCCATTGCCTGACTCAACTACACAGCAAATATTCTCTATAATGCCATCAAATATCTC
AGAATTCTCCCCAACAACATGGTACAGGAGAAGCCATGGGCCACACTCAAATCTGATCGCCGCTCTGGGC
GTCTTTTACTTCTCCGCCAGGTGCTGTGCTCCATCATCGCCGGTACCTTGCACTATCTCTACCTGGCCAC
CTTGACCTGGATGCTGCTGGAGGCCCTGTACCTCTTCCTCACTGCACGGAACCTGACGGTGGTCAACTAC
TCAAGCATCAACAGATTCATGAAGAAGCTCATGTTCCCTGTGGGCTACGGAGTCCCAGCTGTGACAGTGG
CCATTTCTGCAGCCTCCAGGCCTCACCTTTATGGAACACCTTCCCGGTTAGTGCAAATTCTCACAATCCT
TATCTTCTCCGGCATAACCATGGCCATTGCTAGAACCTAGATAGCATTTTTATGACACCTGCTTTTAATT
AGATCAGTAGGGATTTTAGGCTCTATTGGTTGGATTACACTGCAGCTCCTTATTCTTAGCTATTGAGTTT
ATTTCAATTACATTTCAAGCTAGGCATTGTAGAAGAGGGTTTTCGGAAATTTTCTGAATGAAATAAAGGG
CAAGCCTTTGGAAAGCACGAATAGGACAAAGACACTATGTCATAATCCCTAGGGCATCTTTCTGCTAGAA
GATTATAATAATTAAGGAGGGTCAGGCTCAGTGGCTCGCACCTGGAATCCCTGTGCTTTGGGAGGCTGAC
ACAGGAGGATCACTTGAGCCCAGGATCTTGAGACCAGCCTGGGCAACACAGCAAGACCCCTTCTTTACAA
AATTTAAAAAATTAGCAGGGCCTACTGGCACACACCTGTAGTCCTAGCTACTCAGGAGGCTGAGGTGGGA
GGATCGCTTGAGCCCAGGAGTTCGAAGTTGCAGTGAGCTATGATTGCACCACTGCACTCCAGCCTGAGCA
AGAGAGAAAGACCTTGTCTCTAAAAAATAATAATAATAGTTAGGGATAATGCATTTAAATCTGTCTCACT
GAAATAGTAAGAATGAAATCAAAGTGCTCAAGACAGCATTTTTTAAAAATCTAACCTACTTTATCTAAAT
CTTTTAGGATACTCCAATTTTTCATTTGAAAAAGTAGACCTGATTTGAATAACTAAAATAATTGATATTG
TCATAGTTAAGATTTATGAGATGCTTACTTTGTGCCAGGTACTTTTCTAGCTAGTTTGTTTGTTTGTTTG
TTTGCCAATTGCACTGGTTGAAACCTCCAGGACACTGTTGACTAGAACAGCAGACAATTTTGTCTTGCTC
CTGTACTTAGGGAGAAAGCATTGACTCTGTCCTCATTAATGTATGATGTTAGCTGAGGATTTTTCATAGA
TACCCTTTTTCAGGTTAAAGAAATTCTTTTCTATTCCTCATGTGTTCGGTATTTTGATGAAGAAAGAGTG
TTTGATTTTGTCAAATGCTTTTCCGAAGCATATAATTTTTAATTATATGGGGTAAAAATTATATGATCAC
ATAATTAGATAATCATATAATTATATAATTATATTAGATTATTTTATATGTAATTTTTATATAAATATAT
ATCTATGTAATTTATATATTAATATTATATAACTATAAATAATTATATAACATAGCTATAAATAATTATG
TAACTATGAATAATTACATAACATAGCTATAAATAATTATGTAACTATGAATAATTACATAACATAGCTA
TAAATAATTATGTAACTATGAATAATTACATAACATAGCTATAAATAATTATGTAACTATGAATAATTAC
ATAACATAGCTATAAATAATTATGTAACTATGAATAATTACATAACATAGCTATAAATAATTATGTAACT
ATGAATAATTACATAACATAGCTATAAATAATTATGTAACTATGAATAATTACATAACATAGCTATAAAT
AATTATGTAACTATGAATACATAACATAGCTATAAATAATTATGTAACTATGAATAATTACATAACACAG
CTATAAATAATTATGTAACTATGAATAATTACATAACATAGCTATAAATAATTATGTAACTATGAATAAT
TACATAACATAGCTATAAATAATTATGTAACTATAAATAATTACATAACATAGCTATAAATAATTATATA
ACTATAAATAATTACATAACATAGCTATAAATAATTATGTAACTATAAATAATTATATAACATAGCTATA
AATAATTATATAACTATAAATAATTACATAACATAACTATAAATAATTATATAACCATAAATAATTATAT
TACATAACTATAAATAATTATATTACATAACTAAATAATTATATTACATAACTAATTATATTACATAACT
ATAAATAATTATATTACATAACTATAAATAATTATATAACTATAAATAATTATATTATATAACTATAAAT
AATATGATCACATTAAATTGTATAATCATATATTATCATATCATTATAATTATATTATATATGTATATAT
CATATATAATTACATATAATTATATAAGCATAATTATATGCTTCCTTAATTCTTTATTTTATTAATACAC
ATATTAATAGAATAAAGGATTAAAATTATATGATCACATTATAATTATATGGTTATATAATTATATGGTT
ATATATGATTAATTATTTACCTTTGAGTCCTGGGGAATATTATATGGTTATATATGGCTAATTATATGGT
TATATAATTATAATGTGATCTTATAATTATATGATTACATAATTATATAACTATAATGTGATCATATAAC
TTTAATCCTTTATTCTATTAATATGTGTATTACATTGATGGATGATTAGATGTTAAACTAGCCTTACATT
CCTGGGATAATTACCACCTGGTCAGGGTATATAATCCTTTTAATATGTTGCAGGACTTGGTCGGCTAGTA
TTTTTTTGAGGATTTTTGCATTCATTTTCATAAGGGATTTTGGTCTGCAGTTTTCTTTTCTTGTGATGTC
TATTTCTGGGTTTGGTGTCAGAGTAACACTGGCCTCGTAGAAGATTTGGAAAGTGCTTCCTCTTTTTCCG
TTTTTGGTTTTGTGAACTATTAACACTAATCCTCCAAAAGTTAACCATAGAGCTACCATATGACCCAGGT
ATATACCCAAAACAAATGAAAACGATGTCCACAAAAAAACTTGTACATGATGCAGCCATAAAGAATAAGG
AGATCGGCCGGGTGCGGTGGCTCACACCTGGAATCCCAGCACTTTGGGAGGCCGAGGCGAGCAGATCACA
AGGTCAAGAGATTGAGACCATCCTGGCCAACATGGTGAAACCCCGTCTCTACTAAAAATACAAAAGTTAG
CTGGGCGTGGTCGTGGGTGCCTGTAATCCCAGCTACTTGGGAGGCTGAGGCACAAGAATCGCTTGAACCT
GGGAGGTGGAGTTTGCAGTGAGCCAAGATGGCTCCATTGCACTTCAGCCTGGCCACAGAGCGAGACTCCA
TCTCAAAAAAATAAAATAACAAGATCATGTCCTTTGCAGCAATATGGATGGAGCTGGAGGCCATTATTCT
AAGTGAATTAATGCAGGAACAGAAAATCAAATACCGCATGTTCTCACTTATAAGTAGGAGCTAAACATTG
GGTACCCATGGACACAAACAAGGAAACTTCAGACACCGAAGCCTGCTTGAGGGTGGAGAGTGGGAGGAGG
GCGAGGATAAATAAACGACCTATAGAGTACTGTGCTGATGACCAGGGTGACGAAATAATCTGTACACCAA
ACCGCCGCAACATGCATTTTACCTACATAACAAACCTACAAAATGTACCTCTGAACCTAAAATAAACGTT
TAGAAAAATGTTGTACATGAATGCTCATAGAAGCAGTATTTAGAATAGTCAAAAATTGGAAACAATGCCT
ATCACTGATGAATGAATAAACAAAACGTAATATATCCACAATGAAATGTTATTCAGCCATAAAAAGGAAC
GTTAAGAGACCATTCAGTCATTATTTATTTATTTAGAAACACTCTGTCACCCAGGCTGGAGTGCAGTGGT
GCCATCTTGGCTCACTGCAACCTCTGCTCTCTGCTTCCTGGGTTCAAGCAATTCTCATGCCTCAGCCTCC
CGAGTAGCTGAGATTACAGGCATGCGCCACCACACGCAGCTCATTTTTTGTGTGTTTTTAGTAGAGAAAG
GATTTCACCACACTGGGCAAGCTGGTCTCAAACTCCTGATATCAAATGATCCACCCGCCTCGGCTCCCAA
AGTGCTGGGATTACAGGTGTGAACCACTGCACCTGGCCTCATTCAGCCATTTTAAAATGCTGCAATATAG
GTCTTGCTCTGTTGCCCAGGCTGGAGTGCAGTAGTGCAATCACAGCTCACTGCAGTCTCGACCTCCTGGA
CTCAAGCGATCCTCCCACCTCAGTCTCCCAAGTAACTGGGAATACAGGTGTGAGCCACCACACCCGGCTA
TTTTTTTATTTTTTGTAGAAATGGGGTCTTGCTATGTTGCTCAGGCTGGTCTCAAGCTCTTGGTCTCAAG
CAATCCTCCCACCTCGGCCTCCCAAAGTCCTGGTGTGAGCCACTGTGCCTGGCACAACCTTCCATTTTTG
TTTCATTGTTTGAATGTGTCTGATTGTGTGTATCTTCCTTGAGTTGAAATTTTGGGGATCTAAGACCGTG
TTCATTTTCTCCACCACTGTATCTTCAGAGCCAACCACAGACCCTATCACTAAAGAAACACTTAATATTG
TTGACGTAGGAAGGAAGTGAGGAAGTGGTGAGGAGGGATGGAATAGATTTACATAACTGTTCCATATCTC
CAAAAACTGATGCATTTTTTTTAATCTCTCTTTTTTCAGCTGCTGGCTCCAACCAGAAAAGGGATTTATA
TGGGGCTTCCTTGGACCTGTCTGCGCCATCTTCTCTGTGAGTGACATCATCTGAGCATCCTCGCTGCCCC
GCTGAGGGTCATTAGAAAAACAAGATAATAAAGATGGTGACAAGAGCAGGAATGTCCCCTGGGTTGTCAT
GGGTTGGGTTAGGTTTCCTAAGACCACTCTTGAATCTTCAAACTCAACACTGTTACTCAATATTTAAGAA
AAAGACTTACGTGTATAATTCATATTAGACAGGCTAGGATGAAGAGATAATGGGCTTCAGGCCCGGTGTG
GTGGCTCACGCCTGTAATCCCAGCACTTTGGGAGGCCGAGGCAGGCAGATCACTTGAGGTCAGGAGTTCA
AGACCAGCCTGGCCAACATGGTGAAACCCTGTCTCTACTAAAAATACAAAAATTAGCCAGGCGTGGTGGT
GGGTGCCTGTACTCGGGAGGCTGAGACAGGAGAATCGCTCGAACCCGGGAGGCAGAGGTGGCAGTGAGCT
GAGATTGCACCGCCACACTCCAGCCTGGGCAACAGAGCAAGACCCTGTCTGAAAAAAAAAAAAAAAGAGA
TAACAGGTTTCAAAATCTATTCAACAAATCTTCTGTGGTTTTCTGAAGGTGAATTTAGTTCTCTTTCTGG
TGACTCTCTGGATTTTGAAAAACAGACTCTCCTCCCTCAATAGTGAAGTGTCCACCCTCCGGAACACAAG
GTAAGATGGAGAAGGGGGTGATAACCACACAGACATGTATGGTTCCCAAGAGCCAAGCCATGTGCCTCAG
CAGCCACTGAGGTCCCTTCTCAGGGTCTTTTGAAGGGACCCAGAAAAAACAGACGCAGCCATCCTTCTAT
GGCAGCCATCACAGATTCAGTGGTATCCTGTTGAGACCCCATGCAACATGTTTTGTCCATGTCTAATCTC
AGTTTGTGAATCCCAGAGCAACAGGAAGAATCTTCATTGACTGGTAAATGGTATATATACAAAATAGGAT
ACGATTCGATCACAAAACAGAATGAAACCATGTCATTTGCAGCAACATGGATGAAACTGGAGGTCACTCT
CTTAAGTGAAATAAGCCAGGCACAAAAAGACAAATACCACAGGTTGTCACTTACATAGAGAGGAGCCGAA
GAAGCTGATCTTGTGGAGGCAGAAAGTAGAATGATAAAAGTTAGCCGGGCATGGTGGCAGGCACCTGTAG
TCCCAGCTACTCGGGAGGCTGAGGCAGGAGAACCGCTTGAACCCAGGAGGCGGAGGTTGCGGTGAGCCGA
GATCACACCACTGCACTCCAGCCTGGGCAATAGAGTGAGACTCTGCCTCAAAAACTAAAAATAAAAATAA
AGTAGAATGATAGATTCCAGAGTCTGGAAAGGGTATGTGAGTGGGCAAGGCGATGAAGAGAGGTTGGTTA
ATGGGTACAACATACAGTCAGATAGAAGGAATAAGTTATAATATTGGATGGCAGAGTAGGGTGACTCTAG
TTCACAATATGTTGTATATTTCAAAATAGCTAGAGGGGAGAATTTGAAGTGTTCCCAACACATACAAATG
GTAGACACGCGAAGTGACGGATACCCCAAATACTCTGACTTAATCATGACACACTCTATGCATGTTAACA
AAATATCACATATGTCCCCACAAAGATGTACAAACATTACGAATCAATAAACTAAAAGGTTAAAAACTTC
TTTGGACTTTTGTCAGCAGTCCTATAACAAAAGCACAAAGAAGGCCGGGCATGGTGGCTCATGCCTGTAA
TCCCAGCACTTTGGGAAGCTGAGGGGCGGGGGCAGATCACAAGGTCAGGAGTTCGAGACCAGCCTGGCCA
AAGAGACCAGCCTGACCAACGTGGTGAAACCCCGTCTCTACTAAAAATACAAAAATTAGCTGGGCGTGGT
GGCGGGTGCCTGTAATCCCAGCTACTCCAGAGGCTGAGGCAGGAGAATTGTTTGAACCTGAGAGGCAGAG
GTTGCAGTGAGCCGAGATTGCGCCACTGCACTCCAGCCTAGGTGACAGAGCAAGACTCCAACTCAAAAAA
AAAAGTGCAAAGAACACGTACATTTTCTTGCCTGTTCCCACAGCCCAGTCTGCACCTTCATACCGTACCC
TCAGTTCAGGCTCCTCATCACTTCTCATGCAATCCTCCCTCTGGGAAAATCCTAATCAACATTAAATCCT
TTACGGGGGCCAGGCATGGTGGCTCACGCCTGTAATCCTAACACTTTGGGAGGCCGAGGTAGGCGGATCA
CTTGAGGTCATGAGTTCAAGACCAGCCTGGCCAACATGGTGAAACCCCATCTCTACTAAAAATACAAAAA
TTAGCCAGGCATGGTGGTGGGCGTCTATAATCCCAGCTACTCAGGAGGCTGAGGTGGGAGAGTCACTTCA
CCTGGGAGGCAGAGGTTGCAGTGAGCCAAGATCACGCCACTGCACTCCAGCCTGGGTGACAGAGGGAGAT
TCCATCTCAAAAAAATAAATAAATAAAAATAAAAATAATTCCTCTACAGGGTTTGAGCAGTTTGTTGTTC
CAGGCACCCTTGTGCAAAATTAATGAACTTTCTTCTAGATTCCTATCATTATCCTTTCCAAATTTTGTGT
GTCATGCCTGGTTGTATTGGTGATTTGAAGAGACTGTGTAGTTCATGAACACAGAAATCTCTTTGCATAT
TCAAAAATAATCCTTTTTTTTTTTTTGAGACAGAGTCTCGCTCTGTCACTCCCAGGCTGGAGTGCAGTGG
CTCTATCTCGACTCACTGCAACCTCCGCCTCCTGAGTTCAAGCAATTCTACTGCCCCAGCCTCCTGAGTA
GCTGGGATTACAGGCGCCCCCCCGACCACACCTGGCTAATTTTTGTATTTTTTTAGTAGAGACCAGGTTT
CACCATGTTGGTCGGGCTGGTCTCGAACTCCTGACCTCAGGTGATCTGCCTGCCTTGGCCCCCCAAAGTG
CTGGGATTACAGGCATGAGCCACCGTGGCCAACCCCAGGTGGCATTTCTACTGTGGGCTATAGTTGACAG
TAAATATCTAAGCTGACCACAGCCCAGTCTGAGAAATCATCTACAGGGGAGGGCAGGGGACTCCATGGGC
ACAGATGAAATAGCCTTGATTCTCTTTCTCTGGGTGCATCACCAGCCTCCTTTCTTACTCAATCCAGGAT
GCTGGCATTTAAAGCGACAGCTCAGCTGTTCATCCTGGGCTGCACGTGGTGTCTGGGCATCTTGCAGGTG
GGTCCGGCTGCCCGGGTCATGGCCTACCTCTTCACCATCATCAACAGCCTGCAGGGTGTCTTCATCTTCC
TGGTGTACTGCCTCCTCAGCCAGCAGGTACCACTGCCCAGCTCCCACCCAGGACTCTTCCTGTCCTCACT
GCTCTCTGTGAGCTGACCCAGTACACACTTTGCCTCTGCAGGTCCGGGAGCAATATGGGAAATGGTCCAA
AGGGATCAGGAAATTGAAAACTGAGTCTGAGATGCACACACTCTCCAGCAGTGCTAAGGCTGACACCTCC
AAACCCAGCACGGTAAGATCACGCATTGCTCCAGAGCACTTCACTAACCGACCCACCTGAGGAGCATGTG
CCTATCACACAAGGAAACCTGGGAATACAGCAGGCAATGCCCTAGAAAGGCTCGCATCTGAGTACGCCTT
GACTCATTAACCATTAGCAATGATCTCAGTTTAAATGTTTTTTTTTAATCAGTCATAGCCTGTCATCCCA
GCATCACTGTCATCCCAGCATTTGGGAGGCCTAGGCAAGAGGATCACCTGAGGCCAGGAGTGCAAGATGA
CCCTGGGCAACATAGCAAGATCCCATCTCTACAAAAAATAAAAATAAAAATTAGCCAGACATGGTGGCAT
ACACCTGTAGTCCCAGCTACTCCTCGGGAGGCTGAGGTGGGACAATCACTTGAGCCCAGTAGGTTGAGGC
TGTAGTGAGCCATGATCATGCCACTGCACTCCAGCCCAGGCAACAGAGTGAGACCCTATCCCAAAAATTA
AAAAAAAAAAAAAAATCCTTGGCTCTTACTCCTTAGGGAACTGTGCTTAGTTTGACTCTCACACACAAAC
ACTCCTATTGGACACCTACTATGTGCCATGCATGGTTAACTAAATGAGCAGGAATTGATCTGAAGCAGTG
ATGCACATTCTACCTAGAAGCATTTACCCTATCCCTTTCCTCTACATACAAGTCTATCTCACAGTCACTT
GGCACCACCACCCAATAAGGCAAGCATGGGAGGAAATGCTGTACATATTTATGAATTAAACAGAAATAAG
TGTTAACTGGGATTTAGGCTTTCTTAACAAGTCTGCCTCTACCAACTACCACTTAGTAGTGGGTAGTGGG
CAGTGCCTCTACTCAGCACCCTCTACCCACTGCAAAACTCAGAAAGAAAAGCAGTAATGGGGCCAGCGAT
GGTGGCTCATGCCTGTAATCCCAGCACTTTAGGAGGCTGAGGCAGGACAACTGCTTGAGGCCAAGAGTTC
AAGACTAGCCTGAGCAACCCAGTGACACCTCGTCCCTATAAAGCATTTAAAAATTAGCTGAGGGTGGTTG
TGTGCATCTGTAGTCCCAGCAACTCAGGAAGCTGAGGTGAGAGGATCACTTGAGCCCAGGAGTTTGAGGC
TGCAGTGAGCTATGATTGTGCCACTGCATTTCAGCCTGGATGACAGCAAGACCTCTTCTCTAGAAAATAA
TACAAATAACAAAATTAAATAAATAAAGTTTTAAAAACACACTGGGCAACCTGGGAATCAGCGGGTGATG
CCCTAAGAGGGTTCACATTTGAGCGACTCCTTGACTCATTAACCATTAGCAATGATCTCAGTTTAAATGC
TTTTTTATAAATCAGCCAGGAACAGTGGTTCAAGCTTGTAATGCCAGCACTCTGGGAGGCCAAAGCAGGA
AGATCACTTGAGCCCGGGACAACTTGGGGATCACTTGAAGCCATATTATCCTATGCCTCAATGGCATGGG
ATAATTCATGACATTGATGACTAATAACCAACATTTATTAATGTAGACATACAAAGTTCTGTTGTATTTT
TCTCACCTCTTCTTCCATCTTCTCCAGATTATATGTGTAATTCTAACCCCATTTCACTGATGAGCAAAGT
AAGACTACTTGGATTTGTGTCAAATGTACAGAGTCTCGGCCAGGCACAGTGACTCATGCCTGTAATCCCA
GCACTTTGGGAGGCCGAGGCAGGTGGATCACAAAGTCAGGAGATCGAGACCATCCTGGCTAACACGGTGA
AACCCCATCTCTACTAAAAATACAAAAAAAAAAAAAAAATAGCCAGGCGTGGTGGTGGGCGACTGTAGTC
CCAGCTACTCGGGAGGCTGTGGCAGGAGAATGGTGTGAACCCAGGAGGCAGAGCTGGCAGTGAGCCGAGA
TCGCGCCAATGCACTCCAGCCTGGGCAACAGAACGAGACTCTGTCTCAAAAAAAAAAAAAAAAAAAAAAG
AAATGTACAGAGTCTCTTCAGCCAGTGAGATTTAGGCCTCTTGAGTCTTGCTTCACATTTAAAGAGCCCA
TTCAGGGCCAGGCACCGTGGCTCATACCTATAATCTTAGCACTTCAGGAGGCCAAGGCAAGCAAATCACC
TGAGGCCAGGAGTTCGAGACCAGCCTGGCCAACATGGCAAAACTCCATCTTTACTAAAAATACATAAATT
AGCCAGGCATGGTGGTGCACACCTATAATCCCAGCTACTCAGGAGGCTGAGGCACAAGAATTGCTTGAGC
CGGGGAGGTAAAAGTTGTGGTGAGCCAAGATTGCCCCATGCCACTGTACTCCACCCTGGGCAACACAGCA
ATACTCTGCCAAAAAAAAAAAAAAAAAAAACAGCCTATTCAGCCTGGGCAATGTAACGAGACCCCATCTC
TATAAAAAAGTTTTAAAAATTCGCTGGGCGTGGTGGCACATGCCTGTAGTCCCAGCTACTTGGGAGGCGA
GAGGATCGCTTGAGCCCAGGAGTTGGAGACCAACCTGTGCAACATGGCAAAATCCCATCTCTACACAAAA
TATAAAAACTAGCTGGGCATGGTGGTGCATGCCTATAATCCCAGCTGCTTGGGAGGCTGAGGCAGGAGGA
TCACTTGAGCCCAGGAGTTCAAGGCCACGGTGAGCTACGATTATGCCACTGCACTACAGCCTGGGTGACA
GAGCAAGACCCCTTGTGTAAAGTAAATACATAAATAATAAATTTTTTAAAGTCTTGAAAAGAAGCCCACC
ATGCTGCCAGGCACTTTTTCCACATTTGCATTTCTCTCATTACCTTTTTCCTTTTTTATTTTTCTTTTTT
TTTTTTTTTTGAGACGGAGTTTCGCTCTTATTACCCAGGCTGGAGTACAATGGTGCTATCTCAGCTCACC
GCAACCTCCGCCTCCTGGGTTAAAGCGATTCTCCTGCCTCCACCTCCCAAGTAGCTGGGATTACAGGCAT
GTGCCACCAAGCCTGGCTAATTTTGTATTTTTACTAGAGACGGGATTTCTCCATGTTGGTCAGGTTGGTC
TCAAATTCCCGACCTCAGGTGATCTGCCCGCCTCAGCCTCCCAAAGTACTAGGATTACAAGCGTGAGCCA
CCACACCTGGCCTCATTACCTTTTTCATCCAACCTGCACAATCCCTGTTTCCAACACAGGATCTTCTATT
TCCTTTTCTGTTCTCACTAAATGCCCTCACCACCTATGTCACACACACACACACACACAAATCCAAGTCA
TCATGATGACACATTGTAAACTCCATGCTCTCAATCATACAACCAAACATGACCATCCACCTTCCACATT
CCCATATTATTGACTGTCCATCCTCTTTTTTTTTTTTTAGACAGAGTCTCACTCGCTCTGTCACCCAGGC
TGGAGTGCAGTGACACCATCTGGGCTCACTGCAAACTCTGCCTCCAGGGTTCAAGTGATTCTCGTGCCTC
AGCCTCCCGAGTAGCTAGGATTACAGGCACCCGCCACCACGCCTGGCTAATTTTTGTATTTTTAGTAGAG
ACAGGGTTTCACCATGTTGGCCAGGCTAGTCTTGAACTCCTGGCCTCAAGCCATCTGCCCGCCTTGGCCT
CCCAAAGTGCTGGGATTACAGACGTGAGCCACTGCGCCCAGTCCATCCCTGTCTTTGACTCTCCTACAGC
CTTCATACTTTATAAAGACCTTGATGATTACATTGGGTTCACCAAGACAATCCAGGGCAATTTTCCCATC
TCAAGATCCTTTTCATAACCGTATCTACAAAGTCTCTTTCTCCATGTCAGGTAGCATTTACAGGTTCTGG
GGATTAGGGATGTGAACCTCTTTGGGGGGTCATTATTCTGTCTCTCATACCCATATAGTTTGGGTTTACC
TATTGTAGAATCATCAAAACCTGCCTCATATTGTAGATATTTGCATAAATATTTATCACCCTTTCACCCC
ACACAGCCACTAGAAATGATATATACACATTGAAGAAAGGAAACATGTACTTTTGTGTAGGGAATTAGAG
TTAGGTCATCAGATAGCCAACAAAGGCTTCTGAAATTTAAGGTGTGTGATCAGATATTTCTACCAGCTAC
TGACAAAGGACCAAGAAAATCCATTGCATTACTTAATGTCATTTTATCTTCTACTGTTTCCTAAAACTTG
TTATCTCTGGTGTTTGAAAATGTATGTGCCTTGGCCGGACACGCTGGCTCACACCTGTAATCCCAACACT
TTGGGAGGCCAAGGCGGGCAGAGCACTTGAGGTCAGGAGTTCGAGACCAGCCTGGCCAACATGGCAAGAC
CCCACCTCTACTAAAAACACAAAAACTAGCCAGGCATGGTGGTGGATGCTTGTAATTCCAGCTACTTGGG
AGGCTGAGGCACAAGAATCACTCGAACCCAGGAGGCAGAGGTTGCAGTAAAGCCAAGATCGAGCCACTGC
ACTCCAGCCTGGGTGACAGAGTGAGACTATGTCTCAAAAAAAAAAAAAAAAAGAAAAGAAAAGAAAAGAA
AATGTGTGTCTTGCTCTTATTCTGCAGTCTCTGCCATGGCTTTCTGCAGTCTTGTTTCATAAAAACAAAA
TGTCTTGTGATCACTACTGAATAATCTCCTGAAGCACAATATTTATTAATCTCTTGAGCACATATTAATT
ATGCATTAAATACTAACTATATAAGCAAGTGAAGCTGACCATCTCGAACCATAGAAATTACACTGAGTTT
CCTATGAACCTAGGAATAAATAACTCATATTGGAGATGGAAAAGATATGGAAAAAAGGAATTCAGGCTGG
GCGCAGTGGCTCACGCTTGTAATCCCAGCACTTTGGGAGGCCGAGGCAGGCAGATCACTTTAGCTCAGGA
CTTCAAGACCAGCCTGGGTAACATGGCAAAATTCCGTTTCTACAAAAAGTATAAAAATTAGCCAGGCATG
GTGGTGCATGCCTGTAGTCCCAGCTACCGGGAGGCTAAGATTGGAGTATTGCTTGAGCCCAGGAGGTCAA
GTATCCAGTGAGCTATGATCCCATCACTGCACTCCAGCCTGGGCAACAAAGCAAGACCCCTGTCTCAGGG
GAAAAAAAAGGAATTCAGTGCTTTTTCCTTTAAATCTTATGTGGTTAACAATAGATAACACTTCTAACAT
TTCATTAATTTCCCACCTGGCTTCTTTTATGACTCCACATTTAAAGCAACATTACACAAACCCTGGAGCT
CCGAGGTCAAGACATACATTTGGAAACAGATAGATCTTTTTAAATGTTTTTTAATTGATCCATAATAGAC
GTACATAGTTTCTAGGTACATGTGATCATTTGCTACACTCATATAAGCAAATCAGGGTAACTAAGATATC
TATCACCTTAAATACTTATCTTTTCTTTGTACTAATAACATTCAAATTATTCTCTTCTAGCTAGTTTGAA
ATGTATGGTAGATTAATGTTAACTATAATCACCCTACTTCAGATACAACTTTTTTTTTTTTTTTTGAGAG
GGAGTTTTACTCTGTCGCCCAGACTGGAGTACAGTGGCACCATCTTGGCTCACTGTGACCCCCACCTCCC
AGATTCAAGCGATTCTCCTGCCTCAGCCTCCCGAGTAGCTGGGACTACAGGCGCCTGCCACCAAGCCCAG
CTAATTGATTATTATTATTACTACTGTATTCTAGTAGAGACAGGGTTTCACCATGTTGGCCAGGCTGGTC
TCAAACTCCTGACCTCAAGTGATCTGCCCGCCTCGGCCTCCCAAAGTGCTGGGATTACAGGTGTGAGTCA
CCACACCTGGCCCAGATAGATACTTTACTGCAACACATTTACTTCCTCTTAACTTTGGTAACTTGGGGTT
TAGGAGGCATACAAAGATGGGGCTTCTGAGAAAGAACAGGATGGAGCAAAATGAAAGCTGATATGGCCTT
TTGATGTTGCCCTGACAAATACATATGGTAAGAAAACCTTATGATTAAAGACTTCTTGGCCAGGCATGGT
GGCTCACACCTAGAATTCCAGCATTTTTGGGAGGTTGAGATGGGAGGATCACTTGAGCCCAGAAGTTCAA
CAGCAGCTTGGGCTATACAGTGAGACCCCATCTCTACAAAAAATTAAAATTAAAAAATGAGACAGGAGTG
GTGGCACATGCCTGTAGTTGCAGCTACTCGGGAGGCTGAGGCAGGAGGATCACTTGAACCTGGGAGTTGG
AGGTTGCAGTGAGCAGAGATGGTGCCACTGCACTCCAGACTGGGTGACAGAGCAAGGCCCTGTCTCTAAA
AAGAAAAAAAAAAAAAAGACTTCTTAAATTTCTTTTTTTTAATAAATTTTATTGTGTATACACACAATTG
AGGTTTAACCCATGAAGTTATGGGATACATATAGATAATAAAATGGTTATCTATATGTGAAGCAAATTAA
TATATCTATCATCTCACATAGTTACTTATTTTTGTGGCACAAGCAGCTATAATCTACTTATTTAACTAAT
TTCTTATTTCTTTTTCAAAATGGAAATTATAGGTTTTCTTTCTATAGTCATATATATTTCTAAATATATA
TATATTTCTAAATATATAGATATTAAATATATATATTTCTAAATATATAGATATTAAATATCTATATATT
TCTAAATATATAGATATTAAATATCTATATATTTCTAAATATCTATATATTTCTAAATATCTATACTTCT
AAATATCTATATTTCTAAATATCTATATATTTCTAAATATCTATATTTCTAAATATCTATATATTTCTAA
ATATCTATATATTTCTAAATATCTATATATTTCTAAATATATATATTTAGAAATATCTATGTATTTCTAA
ATATCTATATATTTCTAAATATCTATATACTTCTAAATATATCTATGTTTCTAAATATATTTAGAAATAT
ATATATATTTCAGGCCAGCTCAGTGGCTCACACCTGTAATCCCAGCACTTTGGGAGGCTGAGGCAGGCAG
ATCACCTGAGGTCAGGAGTTCAAGGCCAGCCTGGCCTACGTGGTGAAACCCTGTCTCTACTAAAAATACA
AAATTAGCCGGGTGTGGTGGCACATGCCTCTAATCCTGGCTACTCTGGAGGCTGAGACAGGAGAATTGCT
TGAACCTGGGAGGCGGGGGTTACAGTGAGTCGAGGCTGCGCCATTGCACTCCAGCCCAGGCAACAAGAGT
GAAACTCTGTCTCAACAACAAAATACATACATATATATATATTTCAGGTTAACTAGAAAAATCTTCTGAA
TAAGATCTTCCCTCTTTGCCCGTGGAAAATCTGAACAATCTTTGAGCCATCTAGAGGGGAAAGAAAAGAC
TTTGTTCTGTGTGTTTCAAGAAATTCACCATGTCAGCAATATGAAGGATGTTATGGAAGGCGTGCTAGGC
ATTCAATTCCTGCAGAAACCGGAAATCTTCCATGCCCTGCAATGTGCTCATCAAACTCTCAGCATATGGA
CGGCCAGCTGTGGCCCATATCTTGGTCACTCTGAAGCACAATATTTATGAAGCTATAGAACGTTAAGACC
TCTTTCACAGCCTCTCCTTCCTACAAAGACTCCTCCAAATCTTAAAATGAAGCAGGAAAACGAGCCTAAG
AGGACTTTCATACCGACAACATCTGAAAGGACTAGAATGTTCACACCACGATCTGGATTTCTTAATTTTT
TGTTTTTGTTTTTGTTGTTCTCTAGTTCTACGGGTTTGATTATTTAGTCATGTGAAAAATATTGATTACT
CACACATAGATCAAGAGAGACACGGCTCCTGCCTTCATGGAGCTTTTAGGGGAAAATGAAGTGGCTCTTG
CAGCTAGAGTTGACTCAGAAGCCGAAATTCCTAGAAATCAGGTTTCTACTGCTAGGCAATTGAAGTATAA
ACTATTTTATAAACACTGTCTTCTTTCGTCTTCACACCAACATGCAGAAAAGTTTCTAATCTCAGATCGG
GGATGTGCAACAAATTCCATTTCAAAGGAATGACCTGCAAAACTCCTAAATATTCCAAGCAAATGCCCTT
AACCCTGTCTGTTATCTGCTTTCCTTGAACAGAAATTCTACATGACCATAAAACCTCGAAGATGGGTATG
GCACAGTTCATGCCCTGTAATCCTAGCACTTTGGGAGGGTGAGGCAGGAGGATGGCTCAAGCCCAGGAGT
TTGAGACCAGTGTGGGCAACAGAGTGAGAACCATCTCTACCCAAAAAAAAAATTAAAAATTAGCCAAGCA
TGGTGATGATATAGGAGTTAAGGAGAAATCATTTAGGCAAATAGCAAGGGTAGGAAGTCCTCAGTAAGGT
TTTCCATTTAATGAAAAGCAGCCCCCAAAATCATTTTCTTTTCTAACAAAGAACAGCCTGTAAAATCGAG
CTGCAGACATAGACAAGCAAGCTGGAAGCTTCCACGGGTGAATGCCGGCAGCTGTGCCAATAGGAAAAAG
CTACCTAGACTAGGCATGTCCAAAATGGCGGCTCCAAGTTCCCTTCTCTTTGCCAGCCATGTGTACAGTA
AAAAGCAGGCAACATAGTGTCAGCCAAAGCTCATTTGCATAATAAGATTAGGGTGGGGTGGCCAGCTCAC
ATAGGGGTAGGCCCTAGGTAAATCAGACACCGCCTTCTCAAGCCTGTCTATAAAATCTGGTACACTATGA
CGAGGGTCAGATTTCCCATTCAGACGCCCCTCTCCCATGCAAGAGAAAGAGCTGTTCTCCTTTCTCTTTC
TTTTGCCTATTAAACCTCTGCTCCTGGCCAGGCACAGTGGCTCACGCCTATAATCCCAGCACTTTGGGAG
GCTGAGGTGGTCAGATCACCTAAGGTCAGGAGTTCAAGACCAGCCTGGTCAACATGGTGAAATCTTGTCT
CTAGTAAAAATACAAAAATATATGAAATCTCACATAGATGATAATATTAAGTTCCAAAAGCAACTCAACC
TGGTAGATTCTAATTTTTTTTGAGGCAGGGTCTTGCTTTGTCACCCATGCTGGAGTACAATGGCACAAAC
ACTGCTCACTGCAGCCTCGACCTCCCAAGGCCTAAGCAATCCTCCTGCCTCAGTCCCCCTCCAGGTATTT
GAAACTACAGGTGTGTACCACCACACCGGGCTAATTTTTGTATTTTTTGTAGAGACGTGGGTCTCACTAT
GCTGCCCAGGCTCAGGTCTTAATCTCCTGAGCTCAGGCAATCCGCAGGCCTCAGCCTCCCTAAGTGCGGG
GATTACAGGCTTGAGCCACTGCACCTAGCCTCTATTTGTTTTACAAAAGAGAAATTGAGATCCTGAATGT
TAAGTGACTTGCCTGAGGCCATCCCACTAACAGGAGCCAGGGTTAGGATTCAAACCCCATCCAACTGGTC
CCAGAGCTGGAGCTTCTTGCACTGCCCTACACTACCTACCATCTCCATCCTCTGGGCACCTTTTTATAAG
AACCAAAACATTACAGAGCATTGCTTTGTCAACTCAGCTGGGAACATTTCCCAGTGCAACTCACATTTTT
CACTGCTCTGTGCCTGTCCGTATAAGCTCAATGAGTATTGATTTAGGGGCTTTGGAGAACTTTGAATGCT
ACCCCCCAAGTAACCATTGTTGGCAACCTGGTACCTCTACTTTTAGCCATTTCTCCTTCTCTATAAATAG
TGCAGAAGTAACCCACTTGGTAACAGGCATCCTTGCCAAGCCTCCACCACTAGGTCAGTGTAAGAATTAA
AGAAAGAGGAAAGAAACACAAAAAGTGGCTTGATGGTTAAGACAGGTTTATTTTAGAGAAAACACACCTG
AGAGGGGCTGCTGGCTGAATTAGGTTAGAGTCTTTTCTACAGACTAAGAGTGTTTAAGGATTTAGGGTGG
GAGAGTTTCTTAGAGGCTTGGACTGCTTCTGTGTTTTTTTTGTTGTGCTTATATGGGAGGGAGAGTGGTG
TGTTTGCTTTTATACATTTTTCTGCAGCTGTAGGCATACCCCCCAAGTCTGCTTTTAGCTTCCCTATTTT
AGTGCACCTGGAGGGAAAGGAATGTGCTTATTAAGGCCCACTGTTTTACTGGGGCCCATTGTATGAGGGT
GAAGTTTGGCAGTTACCCAAGAGACTTTTCCTCCACCTTCCTCTGTGCCCGAGCTGTTTTATCTGCATTT
TACTGTCTGCTTTTTTTGGCTGCTTATAGTTTTTAAAAAAGTAATTTCCTTAAATCCAGAAGGCTAAAAA
TGAAGCTGAAACTTAAAGTGGCGGTGTTTGTCCAAAATAACGGGGCTCCTGCTCTGCCAGTCAGTACCCT
CAAGTCACTCCTGATCCTCAACCTCCATGCCTAAGGCTGGTTCAAGAGACCACATAATATCTGCCTTTTA
TTACATACATGATGGGTGCATGGGATTCTGCGTGCCCTTTGCTTGATATAGACTGCTAAGGTGAGATGGG
GAATATCAGAGTCAGCTGCTGCTTGAGGAAGCAGAACACACAGCTGGAGGCTTGGAACATGTGGGTCCCT
ATGAGTGTAGAGCCCATATCCCCATAGAGTCTACCTAGAGCAGGGGTCGCCAAATGTTTTCTTAAAGAGC
CTGATAGTGTATATGTTAGGCTTTGTGAGCCAGGTATTTACAGCAACTCAATTCTACCACTGTGGTATGA
AAACAGCTATAGACAATCATAAATGAATGATCATGGCTATGTTTTAATAAAACTTTACAGACACTGAACT
TGAACTTCCATTGTGATATGAAAACAGCTATAGACAATCATAAATGAATGATCATGGCTATGTTTTAATA
AAACTTTATGGACACTGAGCTTGAACTTCATATATCAGTCATGTGACACAAAATATCATTCTTCTTTTTA
TTTGTTTTCACCCATTTGAAAAATGTAAAAACTATTCTTAGCTGTACAGAAACAGATGGTGGGTCAGATT
TGACTCACTGCCCATAGTTTCCAGACCATGATGTTCAGGTTCATGGAAGCACTTCACTCTACACTAATTT
ATTCATCCATTCATCACATATGTGATGAGCACATACAGACACACCTCAGAGATATTGCAGGCTCCACTCC
AGATCACCACAATAATGTGAATATTGCAATAAAGCAAGTCATACACATTTTTTGGTTTCCCAGTGTGCAT
AAAAGTATGCTTACCCTATACTGTAATGTGTGCAATAGCATTATGTCTTTTTTTTAATGTACATACTTTA
ATTTAAAAATAGTTTATTCCTAAAAAATGCTCAAGCAGGGACACAAAGTGAGCACATGCTGTTGGGAAAA
TGGCACCTACAGACTTGCTCGACACAGGGCTGCCATAAACGTTCAATTTGTAAAAAATGCAATATCTGTG
AAGGGCAATGAAATGAGGTATTCCTATACTCTCGTGCAAGGCATAATGCTAAATTGAGAGAAACTAAAAC
ATATAAGACAGACATGATCCTACATCCAGGAATATTGATGATTTGTAAGATGGAAAATGAGGAGGAAGGA
AAGGTGCAGTGCAATGGACTCTGTGGTCTGGGGGACACACAGCCCCTTTAAGGACATGGCAGAAGTGAAA
CACAGACGAGGTTGGGGAAGGAAGGTCAGAGGAAACTCTAGGGTCAGCTGATGGCAGCATCCATTTGCCC
CCATATCTTCACTTTTTAAATTTATTAAGTCCATTCTCTTTATTTATGATGGAAATAATGTTAATTTGCT
CATGGGTTGTGTTTGAATATTAAGTAAGAAAATAAAGATTAAGCTCTGGTATGTAGTAAGAAACCCAAAG
ATAAACAAGAGTTAAGGGGCAAGTAGGCAGCAGAGTTTCCCATCTCATTTCTCCAGAATTATATCATCCA
GTTTGAAACATGAAATACGGCTTTAAGGTAGAGTTTGGCTTCTCAGCTGGATTTTAAGTACAAGACCACG
CCCACCAAAGACAGCAGACAAAGGAGCTAGGGTACACGTTTGAAGAACCCTCTCCCTCCAGCTCTCCACC
GTGGGGGTCCACTGTGAGCAGTTTCCCTGGCTGTTTTCCCCCTTTCTGCATCTAACAGTCATTGCACGGC
TGGGGAAACAAAGACCCACACAGGCCTTAGTCTCCTTGCGGGGAACCTACAATCCGGAAGCCAGAAGTAT
GTGACTAAGCTCCCGGAAGCCGGAAGTTAAGGAGCCCGGATGTCACGGAACTGGAAGTTTGAAAGTCCAA
GGCCACTGACCTGCAAGCTTCAGAATCTGCAGATTTGGAGTCTTCAAGCCCCAGTAGTGATCCTTTGATA
TCCAGAGGTTGGATTCTAGCGATCTGAACATGCAAGAGAGTACTGGATGTGGAAATTCTTCCAAATTAGT
TGGGTGGCTGAAAAGGGAATTATAAGGAGGAAGAATGAGGGAAAGAGAAGAGAAATTGAATGAATAAAGG
GGCCAATCCTTGGGGGGAGGGGGCAGGAGGGGCGGTGCTTGAGACCAGGGCAGCATGTAAGGGACTGCCT
ATGAATGTCCTAAATATCTGCTATTTAGGGCATTCTGGGATGGGAGTTTGAAAGACTGATGGTGGTGTGT
TTTGCAGCCACATGAATGCAGCTGGAACCATCATCCTAAGTGAATTAACTTAGAAAACCAAATACCGCAT
GTTCTCACCTGTAAGAGAGAGCTAAATATTGGGTACACGTGAACATAAAGATGGGAGCATTAGACAATGG
GGACTACTAGATGGAGGAGGGAGGGAGGAGGGCAAGGGTTGAAAAACTACCTATCAGGTACTATGCTCAC
TAACTGGATTCATTTGTACCCCAAACCTCAGCATCATGCAATATACTCTTGTAACAAACCTAAACGTGTA
CCCTCAGAATCTAAAAATAAAAGTTGAAATTAAAAAAATCACTGTTGATAGACATTTGGGTTGTTCCCAG
TTTGGGTTATTACAAACTGCTTTGCTAGCTTTAACTTTTTTTAAATAATAAACTCTTAAATTTACAGTGC
AAGAAATACAGAAAGAGAGAGGAAGACAAAGAAAAAGAAAGGAGATAGAGAAAGGAAAAGCAAAGAAAGA
AAAAGAAAAAAGTGAAAGAAAGAGAGGAAGGGAGGAAGGAAAGAAGGAAGGAAGGACAGACTGATGGTGG
GATAAGAGAGAAGGGGCTGGGAAGAACTAAGTGGAACATTCTGAAAATGAAGACAGTGGCCGGGCACGGT
GGTTCACACCTGTAATCCCAGCACTTTGGGAGACTGAGGCAGGCAGATCACCTGAGGTCAGGAGTTCAAG
ACCAGCCTGACCAACATGGTGAAACCCCATCTCTACTAAAAATACAAAAATTAACCAGGCATGGTCACGG
GCACCTGTAATCCCAGCTACTCAGGAGGCTGCGAGGCAGAAGAATCACTTGAACCTGGGAGGCAGAGGTT
GCAGTGAGCAGAGATTGTGCCATTGTACTCCAGCCTGGGTGACAGAGCGAGACTCCATCTCAAAAAATAA
AAAATAAAAATAACTCTCTTCTCTCTATTTTCCTTCAATAAATTCTCTTTTTGGCTAAAGTAGTCATTTT
AGCCAAAATCTTTCTCTCAAGACAACAAAGAACTGAGGATTTCTGCCCTTTCCAGTATACAACTAGCAGG
GATCCCACTGGCATTTTTCTTTGGACAGATAAAATTGGATGTATTTATCAAGAGCAACATGATGTTTAAT
GGATGTATACATTGGGGAATAACCAGATCTAGCAAATTAATGTATGCCTCGTCTCATATAGTTATTATTT
GGTGGTGAGAACAATTTACATCCACTCTCTTAGCATTTTTTAAGAATATAATTTATTATTAACTATAGTC
ACCATGCTCTACAATTGATCTCCTGAACTTCTTCCTCCTGTCTAACTGAAATTTGTATCCCACCGGGCGC
AGTGGTTCACACCATAATCCCAGCACTTTGGGAGGCTGGGGTGGGCAGATCGCTTCAGCCCAAGAGTTCA
AGCCCAGCCTGGGCAAAATGGAGAAACCCTAACTCTACAAAAAATATTAGCCAGGCATGGTGGTGCATGC
CTGTGGTCCCAGCTACTCTGGAGGCTGAGGAGGAAGGATCACTTGAGCCCAGTAGTTTGAGGCTGCAGTG
AGCTGTGACCACACCACTGCACTCCAACCTGAACAACAATTAGACCCTGTCTCAAAAAACAAAAAAAAAA
AGTGTATCGTTTGACCAACATCTCCCCAACCCAAGCTACTGGCATTTAGTTAATAGTCCAGGGATGCTGC
TCAACAGTCTTCAGTGCATAGGGCAGTTCCTACAGCAAAGAATTATCTGGCCAATAAGGTCACAGTTGAG
AAACCCTGACTGTAGGATGCAATGAATCTCTCGGTTTCTCTTCAAAGGTTCAGTCTGTTAACTTCCTTGT
CTCTATGCCTCCCTGCCCCAAGTTACTATAAACAGCCTTCCCGTCGGCACTAATCAATAACTCACATCTG
TTCCCTCGGTCACGCATTCTGCACCCGTTCCACCCCTCAAAACCACACACATACCCTTCAAAACTGCACG
TCCCACCATTGTAACTCACGTCCCCTTCCCCCTTCTTTATTTGAGAGAAATATTCGCAAGTAGCCAATCG
GGTCAGCTTACATTGTGCCGACCCCAGCCCATGGGGGAAGGACATGGAGGCAGGGATTGCGTTAAGGATA
TAAAACCCCCCTGGCCTCCTTTGTTCTCTGTGCTCTTGGATCTTGATTGACGTGGGTGGCACCCTTCTGC
AGAAGTAAACTGCCTTGCTGAGAGAACTTTTGCCTCAGTGCTGGTTTCACTTTGCAGCACCCAGCATTTA
TTGCTAGAGCATTTTTATATTCAACATGACCTAAGGTGTAAAGGGATTCACATGGAAGTTTTTGCCATTT
TCTTCATCCTTTCCATTGTCGTCATAGGTCACTCATTCCCAGGTCTAGAGAAAGTTTGATGCAGTAACTG
TTGTTTGAGATGAGTGGCTTCACCTGCTCATCTTTCTATTGAATACATTTATAAGTGATGTGCTTCAAGA
AATTCTTCAAAGCTAAACATAAGACAAAGTTTCTACATATCCTAGCAAGATAACATGACCATTTAAAAAC
TGATATGAAGCTACAGAAGAGACCTCAGGGATGAGAGGAGAAGACCACCCCCACAACTTCAGCCAGACCT
CAGAGAAACAGACTGTCATCACACCCTAGCATAAGAAGAGACACTAAGCAGCGAGGTTTCTGTCTTCAGC
CTGTAGATAGAAAAAATATTCGAAGAATTATCTCATGCCTTTACCAAGTCTCAGCTCAAATTTTGCAGCT
TGAGTATTCTGAAAAAAAAATTTTTTTTTGAGACGGAGTCTCGCTCTGTCGCCCAGGCTGGAGTGCAGTG
GCACAATCTCGGCTCACTGCAAGCTCCGCTTCCCAGGCTCACGCCATTCTCCTGCCTCAGCCTCCCGAGT
AGTTGGGACTACAGGCGCCCACCACCACACCCGGCTAATTTTCAGTAGAGATGGGGTTTCACCGTGTTAG
CCAGGATGGTCTCGATCTCCTGACCTCGTGATCCACCTGTCTCAGCCTCCCAAAGTGCTGGGATTACAGG
CGTGAGCCACCGCACCTGGCCAAATATATTTTTAAGTACTGGAAATTTTTATAAACTCTGAATTCCAAGG
TAAGATAGCTCAGTTTACTTTATAAAGGTGTACCTGCATGCATGTATACATAAGCCATTGATTTTCCACC
ATTGTGGGCAAGAGGGTATAAGGTGAGACTTAACATTAAATGGGATTAAAGGAAATGATTAGGATACTGG
GACTCTTAAGTTTGTAAATCACTGCCTCAGATATTTGGTTATATTTAACTAATAGGTACATGATATAGTT
TGGATGTTTGTCCCCTCCAAATCTCACACTGAAATGTAATCCCCAGTGCTGGAGGTGGGGCCTGGTGGGA
GGTGTTGGATCATGGGGGCAAATCGCTCATGAATGGTTTAGTAATCCCTTTGCTGTAAGTCAGTTCTTGC
TCTGGTAGTTCACACCAGATCAGGTTGTTTAAAAGAGTATGGAACGTCTCTACTCTGTCTCTTGCTCCTG
GTCTCACTATGTGATGTGCCTGCTCCCGCTTTGCTTTCTGCTGTGATTACAAGCTTCCTGAGGCCCTCAC
CAGAAGACAAGCAGATGTTGGTACCATGTTTGTGCAGCCTGCAAAACAGTGAGCCAATTAAACCTCTTTT
CTTGGGCCGGGCTTGGTGGCTCACACCTGTAATCCCAGCATTTTGGGAGGCCAAGGCGGGCGGATCACAA
GGTCAGGAGATCGAGACCATCCTGGCTAGCACGGTGAAACCCTGTCTCTACTAAAAATACAAAAAAATTA
GCCAGGCGTGGTGGCGGACACCTGTAGTCCCAGCTGCTCAGGAGGCTGAAGTGAGAGAATGGTGTGAACC
CAGGAGGCGGAGCTTGTAGTGAGCCAAGATTGCGCCACTGCACTGTCTGGGCAACAAAGCAAGACTCCAC
CTCAAAAAAAAAAAAAAAAAAAAAAAAGCCTCTTTTCTTTATAAATTACCCAGCCTCAGGTATTCCTTTA
TAGCAACGCAAGAACAGGCTAATACAGAAAATTTCTGAAAATGTGAAAGCAGCTTTAGAACTGTGTAACA
GGCAGAAGTTGGAAGAGTTTGGAGGGCTCAGAAGGAAGAAGGAATATGAGAGAAAGTTTGGAACTTCTTG
GAGACTGGTTAAAGGGTTGTGACCAAAATACTGGTAGAGATATGGACAGTGAAGGTCAGGCTGAGGAGGT
CTCAGATGAGGAACTTACTGGGAACTGGAGCAAAGGTCAGCCTTGTTATTCCCTGGCAAAGGACCTGGCA
GCATTGCGTCTATGTCCTAAGGCTTTCGGGAAGTTTGAACTTAATAGTAATGACTTAGAGAGTGTCTGGT
GGGAGAAATTTCTAAGCAGCAAAGTGTTCAAGGAGAGGCACGGCTGCTTCTAACAGCTTACAGTCAGATG
TGGGAGCAAAGGAATTAAAGTTGAACATTATATTTAAAAAGGGAAGCAGAGCATAAAAACTTGGAAAATT
CTCAACCTGGCCATGCGGTAAAGAAGGAAAGAGCATTTTCGGGGGAAGAATTTAAGAGGGCTGCAGAACC
ACCACTTGCTGAAGAGACTAACGTGACTAAAAGGGAGCCAAGTGCTAAGATCCAAGACAAAAGGAAAAAG
GCTTTGAAGGCATTTCGGGGATCTTCCAGGCCACCTCTCCCATCACAAACCCAGAAGCCTAGGAGAAAAG
AATGATTTAGCGGGCCAGTTCCAGGGCCCCGCTGCCCTGTGCAGCCTTTGGACACCATTCTCCACGTTCC
AGCCACTCAAGGCTCCAGCCTTGGCTCAAAGGGGCCCAGGTATAGCTTGGGTTGCAGCTCTGCAGGTTGC
AAGCTGTAAGCTTTGGTGGTTCCAGGTAGTATTAAGCCTGCAGATGCTCAGATTGCAAGCATGAAGGAGG
CTTAGTGGCTTCCACCTAGATTTCAAAGGATGTATGGAAAAGCCACGGTGCCCAGGAAGGAGACTGACAT
TGGGGCATAGCTCCCACAGAGGAACTCTATTAGGATTGGATTCCCCCGTACCCCCACAAGAATCCCTACC
AGGGCACTGCCTAGTGGAGCTGGAGGGCCACCTCCCTCCAGACCCCAGAATGGTAGATTCACTGGCAGCT
TGTGCCCTCTGCCTGAAAAACCTGCAGGCACTCAACTCCAACCCATAAGAGCAGCCATGGGGGTTGCACC
CTGCAAAGCCACAGGGCTGGAGCTGCCCAAAGCCTTGAGATCCCATATCTTGCACTAGTGTGTCTTGACG
CAGGACATGGAGTCAAAGGAGATTTTGGAGCTTTAAGATTTAATGACTGCTCTTCTGGGTTTACAACTTG
CATGAGGTCTGTTGCCTTTTTCTTTTGGCCTGTGTCTCCCTTTTGAAATGAGAATGTTTAACCAGTGCCC
ATACCACCATCTTCTCTTGGAAGTAAATAACATGTCTTTGATTTCACAGGCTCACGGGGAAGGAATTCAT
CTTCAGATGAGACTTTTGACTTGGGACTTGGGACTTTTGATTGAGTTGATGGTGGAACAACTTATGACTT
TGGGGAGGTATTTAGAAGGGATGATTGTATTTTGCAATTTGAGAAGGTATTGAGATTTAAGGGGCAGGGG
CAGAATAATATAGTTTAGATGTTTGTCTCCTCCAAATCTCTTGTTGAAATGTAATCCCCAATATTGGAGA
TGGGGACTGGTGGGACGTATTCGGATCATGGGGGCAAATCCCTCATGAATGGCTTAGTGTCATCCCCATG
GTGCTGTCAGTTCTCACTCTAGTAGTTCATGCGAGATCTGGTTGTTTAAAAGAGCATGCAATCTCCCCTG
CCTGCTCGCTCCCATTCTCATCATGTGATGTTCCTGCTCCTGCTTCACTTTTTGCCATGATTGTGAGCTT
CCTGAGGCCCTCGCCAGAAGCTGAGCAGAGGCTGGTGCCATGCTTGTAGAGCCTGCAGAACCGTGAGCCC
GTTAAACCTCTTTTCTTTATAAATTACCCAGTCTCAGGTATTCCTTTTTAGTAATGCAAAAACAGACTAA
CACAGTACATCTCTCCCTAAAAGTGCTTGGTATTTGCCTACATTTATGAAGAACATAGCATTAAATACAG
CACCAAATAGGTGCTCAATATTTTCACGGAAAGAATTAATGCACATGTGTAAGAATCAAGCAGCACAGTC
ACCTAATGAGGAATAGCCAAATTTTTAAACAAAGTTTTTCTTTTTGAGACAAAATTTCACTCTTGTTGCC
CAGGCTGGAGTGCAATGGCATGATGTCGGCTCACTGCAACCTCCGCCTCCTGGGTTCAAGCAATTCTCCT
GCCTCAGCCTCCAGAGTAGCTGGGATTACAGGCTCCCGCCACCACACCCAACTAATTTTTGTATTTTTAG
TAGAGATGGAGCTTCACCATGTTGGCCAGGCTGGTCTCGAGCCCCTGACCTCAGGTGATCCACCCGCCTC
GGCCTCCCAAAGTGCTGGGATTACAGGTGTGAGCCACTGCGCCCGGCTTAAACAAACTTTTTAATGGAGA
AAATGTCATCCAATAAATCAACCAAGTGTGACCCATCCTCGTGTGAGCCATCCTAGAACATCACCCACTG
TGGACCAGGTTGCCAGTGTTATAACCATGTGCTCCCCAAGTGAATCAGTAAGAGAAGCTATGTGGGGAAG
AGCAATGTATGATCTTACTGTGCTAGGTTTGAAGGCATCAGGCACAGCCCTTCTAAAGTGTGTGTATTTC
TCAGACCTAGCTCTTCTCATTCTGATCTTTTTGAACCATTTCTTGTTTTGTCCCCAGACCTGCTAGAGGC
AAAGCATCTTCTGGGTCAGCTCATTAAGATAGGCAAGGACAAGAAGAGTCTTGAGTGGGACCTAGACAGT
GGCACCTGTTGACTGAAAAGGCAGTACACCAAAAGATGGAGACACCCTGCAGGGTGTTGATGATGGTGAA
GAGATAGGCAAGAGCATAGCAACTGGGCCCACCTGCAGAATGCCCAAACACCAGGTGCAGCCCAAGATGA
ACAGCTGATCTGTCACCTTGAATGTCAGCATCCTGGATTGGATGAGAAAAGAGGCCCATAATATACCCAT
AATAACAAAGGCTATTTCATCTCCATCCCATGGATATCTCTGCCCTCTCCTGTTGGTCATTTCTCACACT
TGAGTTTTGATCAACCTAGATCTTTACTGTTGATAATTCCCCACAAAAGAAATGCCATTTGGATCCAGGA
AAGCATTTTAACTTGGACCAAGGAGAGTAGTGACACTTCATAATATGGGTTGCAAAAGAAGTGTGATGAT
CAACACAAAAAAACAGTTGCATTTCTATGCACTAACAATGAACAACCCAAAAAGGAAATTGAGAAAACAA
ATCCATTTACAACAGCATTAAAAACAATAAAATACTTTGGAATTAACTTAACCAATGAGATGAAGAACCT
GTACACTGAAAGCTAAAAAGCATTGCTGAAAGAACTTAAAAGAGACACAAATAGACATACTATGTTCACG
GGCTGAATGACTTAATATTTTTGCAGAAACAGAAAAATATCCATCCTAAAACTCATATGAAATCTCAAGA
ATACCAAACCAAGAAGTCAAAACCATCTTGAAAAAGAACAGAGTTGGAGGTCTCACACCACACTTATTTC
AAAATTTATTGTAAAACTACAGTAATCAACACAATACGGTAATGGCATAAAGATAAGACATATAGACCAA
TGGAATCGAGGAGGGAGCCCAGAAATAAAACCTCACATTTATAGTCAAATGAATTTTGACAAGGGTGCCA
AGACCATTCAATGGGGAAAGGACAGTCTTCAACAAATGGTGCTGGGAAAACTGGATATGCACATGCAAAA
GAATGAAGTTGGACTCTTTTCTTACTCCATATATAAAAATTAACTTGGCTGGGCATGGTGGCTCACACCT
GTAATCCCAGCACTTTGGGAGGCTAAGGCAGGCAGATCACAAGGTCAGGAGATCAAGACCATCCTGGCTA
ACATGGTGAAACCCCATCTCTACTAAAAATACAAAAAAATTAGCCAGGCATGGTGGTATGCGCCTGTAGT
CCCAGCTACTCAGGAGGCTGAGGCAGGAGAATCACTTGAACCGGGGAGGCGGAGGTTGCGGTAAGCCAAG
ATTGCGCCATTGCACTCCAGCCTGGGCAACACAGCAAGACTCCGTCTCAAAAACACAAAAACGAACAAAC
AAACAAACAGAAATTAACTCATAATACATCAAAGACCTAAATGCAAGAGCTGAAACAATAAGACTCTTAG
AAGAAAACATAGGGGGTAAGCCTCACGACATTAGATTTGCAATGATTTTTTGGATATGACATCTAAAGAA
AACAAAAAATATCTATAAGTTGGACTACACCAAAATCTTAGACTTCTGCATATCAAAGGATACAATTAAT
AAAGTAAAAAGGTAACCCCTAGAATGGGAGAAAATATTTGCAAATCATATATCTGATAAGGGGTTAATAT
TCAGAATATATAAAGAACTTCCACAACTCAACAACAACAAAAAACTAATTTAAAAATGGTCAAAGGACTT
AGACATTTTTCCACAGATGACATACAAATGGTCACAACTAAGAAGGAAAAGCCCAACGTGTGTTCTTCCA
GCCTCTGCTTCTGTCATGTTTGTCAACACTCCATTGGCCAAAGAAAGTCACATGACCAAGCTCGTCTTCA
AGGTTTGGAGAAATAAACTCCACCTCTTGATAGGAGGAGCTGCAAAGTCACATGGAAAAGACATATACAT
AGAGGGATAGAAGAAGTCATTGTGGCAACCATCTACCACTGCATCATTACTT

The present disclosure provides a number of CD5 target sites and corresponding gRNAs that are useful for targeting an RNA-guided nuclease to human CD5. Table 12 below illustrates preferred target domains in the human endogenous CD5 gene that can be bound by gRNAs described herein. The exemplary target sequences of human CD5 shown in Table 12, in some embodiments, are for use with a base editor, e.g., ABE.

TABLE 12
Exemplary base editor target site sequences of human CD5 are provided, as are
exemplary gRNA targeting domain sequences useful for targeting such sites. For each target
site, the first sequence represents the DNA target domain sequence, the second sequence
represents the reverse complement thereof, and the third sequence represents an exemplary
targeting domain sequence of a gRNA that can be used to target the respective target site.

| gRNA | | | | | |
|---|---|---|---|---|---|
| Name | Target domain sequence | PAM | Codon | Amino Acid | consequence |
| g40 | TGGAACGGGTGAGCCTTGCC (SEQ ID NO: 141) | TGG | TCC/CCC | S->P | missense_variant |
| | GGCAAGGCTCACCCGTTCCA (SEQ ID NO: 204) | | CGTTCC/ | RS->RP | |
| | UGGAACGGGUGAGCCUUGCC (SEQ ID NO: 205) | | CGCCCC | | |
| g41 | CTGGCACTTCGAGTTGGAAC (SEQ ID NO: 142) | GGG | TGC/CGC | C->R | missense_variant |
| | GTTCCAACTCGAAGTGCCAG SEQ ID NO: 206) | | | | |
| | CUGGCACUUCGAGUUGGAAC (SEQ ID NO: 207) | | | | |
| g42 | CCGTTCCAACTCGAAGTGCC (SEQ ID NO: 143) | AGG | AAC/GAC | N->D | missense_variant |
| | GGCACTTCGAGTTGGAACGG (SEQ ID NO: 208) | | | | |
| | CCGUUCCAACUCGAAGUGCC (SEQ ID NO: 209) | | | | |
| g43 | CCTGGCACTTCGAGTTGGAA (SEQ ID NO: 144) | CGG | TGC/CGC | C->R | missense_variant |
| | TTCCAACTCGAAGTGCCAGG (SEQ ID NO: 210) | | | | |
| | CCUGGCACUUCGAGUUGGAA (SEQ ID NO: 211) | | | | |
| g44 | CGTTCCAACTCGAAGTGCCA (SEQ ID NO: 145) | GGG | AAC/GAC | N->D N->G | missense_variant |
| | TGGCACTTCGAGTTGGAACG (SEQ ID NO: 212) | | AAC/GGC | | |
| | CGUUCCAACUCGAAGUGCCA (SEQ ID NO: 213) | | | | |
| g46 | CTCGAAGTGCCAGGGCCAGC (SEQ ID NO: 146) | TGG | AAG/GAG | K->E K->G | missense_variant |
| | GCTGGCCCTGGCACTTCGAG (SEQ ID NO: 214) | | AAG/GGG | | |
| | CUCGAAGUGCCAGGGCCAGC (SEQ ID NO: 215) | | | | |
| g47 | GAAGTGCCAGGGCCAGCTGG (SEQ ID NO: 147) | AGG | AAG/AGG | K->R | missense_variant |
| | CCAGCTGGCCCTGGCACTTC (SEQ ID NO: 216) | | | | |
| | GAAGUGCCAGGGCCAGCUGG (SEQ ID NO: 217) | | | | |
| g48 | GGTAGACCTCCAGCTGGCCC (SEQ ID NO: 148) | TGG | TAC/CAC | Y->H VY->AH | missense_variant |
| | GGGCCAGCTGGAGGTCTACC (SEQ ID NO: 218) | | GTCTAC/ | | |
| | GGUAGACCUCCAGCUGGCCC (SEQ ID NO: 219) | | GCCCAC | | |
| g49 | CCAGCTGGAGGTCTACCTCA (SEQ ID NO: 149) | AGG | CAG/CGG | Q->R | missense_variant |
| | TGAGGTAGACCTCCAGCTGG (SEQ ID NO: 220) | | | | |
| | CCAGCUGGAGGUCUACCUCA (SEQ ID NO: 221) | | | | |
| g50 | CCTTGAGGTAGACCTCCAGC (SEQ ID NO: 150) | TGG | CTC/CCC | L->P | missense_variant |
| | GCTGGAGGTCTACCTCAAGG (SEQ ID NO: 222) | | | | |
| | CCUUGAGGUAGACCUCCAGC (SEQ ID NO: 223) | | | | |
| g51 | CTGGAGGTCTACCTCAAGGA (SEQ ID NO: 151) | CGG | GAG/GGG | E->G | missense_variant |
| | TCCTTGAGGTAGACCTCCAG (SEQ ID NO: 224) | | | | |
| | CUGGAGGUCUACCUCAAGGA (SEQ ID NO: 225) | | | | |
| g52 | AGGTCTACCTCAAGGACGGA (SEQ ID NO: 152) | TGG | TAC/TGC | Y->C | missense_variant |
| | TCCGTCCTTGAGGTAGACCT (SEQ ID NO: 226) | | | | |
| | AGGUCUACCUCAAGGACGGA (SEQ ID NO: 227) | | | | |
| g53 | CCATGTGCCATCCGTCCTTG (SEQ ID NO: 153) | AGG | ATG/ACG | M->T | start_lost |
| | CAAGGACGGATGGCACATGG (SEQ ID NO: 228) | | | | |
| | CCAUGUGCCAUCCGUCCUUG (SEQ ID NO: 229) | | | | |
| g54 | CCTCAAGGACGGATGGCACA (SEQ ID NO: 154) | TGG | AAG/GAG | K->E | missense_variant |
| | TGTGCCATCCGTCCTTGAGG (SEQ ID NO: 230) | | AAG/GGG | K->G | |
| | CCUCAAGGACGGAUGGCACA (SEQ ID NO: 231) | | | | |
| g55 | ACATGGTTTGCAGCCAGAGC (SEQ ID NO: 155) | TGG | ATG/GTG | M->V | start_lost |
| | GCTCTGGCTGCAAACCATGT (SEQ ID NO: 232) | | | | |
| | ACAUGGUUUGCAGCCAGAGC (SEQ ID NO: 233) | | | | |
| g58 | TTTGCAGCCAGAGCTGGGGC (SEQ ID NO: 156) | CGG | AGC/GGC | S->G | missense_variant |
| | GCCCCAGCTCTGGCTGCAAA (SEQ ID NO: 234) | | | | |
| | UUUGCAGCCAGAGCUGGGGC (SEQ ID NO: 235) | | | | |
| g59 | TGGAGCTCCGGCCCCAGCTC (SEQ ID NO: 157) | TGG | TCC/CCC | S->P | missense_variant |
| | GAGCTGGGGCCGGAGCTCCA (SEQ ID NO: 236) | | | | |
| | UGGAGCUCCGGCCCCAGCUC (SEQ ID NO: 237) | | | | |
| g61 | GGGCCGGAGCTCCAAGCAGT (SEQ ID NO: 158) | GGG | AGC/GGC | S->G | missense_variant |
| | ACTGCTTGGAGCTCCGGCCC (SEQ ID NO: 238 | | | | |
| | GGGCCGGAGCUCCAAGCAGU (SEQ ID NO: 239) | | | | |
| g62 | CCGGAGCTCCAAGCAGTGGG (SEQ ID NO: 159) | AGG | AGC/GGC | S->G | missense_variant |
| | CCCACTGCTTGGAGCTCCGG (SEQ ID NO: 240) | | | | |
| | CCGGAGCUCCAAGCAGUGGG (SEQ ID NO: 241) | | | | |
| g63 | CCTCCCACTGCTTGGAGCTC (SEQ ID NO: 160) | CGG | TGG/CGG | W->R | missense_variant |
| | GAGCTCCAAGCAGTGGGAGG (SEQ ID NO: 242) | | | | |
| | CCUCCCACUGCUUGGAGCUC (SEQ ID NO: 243) | | | | |
| g65 | AGACTTTTGACGCTTGACTG (SEQ ID NO: 161) | GGG | GTC/GCC | V->A | missense_variant |
| | CAGTCAAGCGTCAAAAGTCT (SEQ ID NO: 244) | | | | |
| | AGACUUUUGACGCUUGACUG (SEQ ID NO: 245) | | | | |
| g66 | CAGACTTTTGACGCTTGACT (SEQ ID NO: 162) | GGG | GTC/GCC | V->A | missense_variant |
| | AGTCAAGCGTCAAAAGTCTG (SEQ ID NO: 246) | | | | |
| | CAGACUUUUGACGCUUGACU (SEQ ID NO: 247) | | | | |
| g67 | GCAGACTTTTGACGCTTGAC (SEQ ID NO: 163) | TGG | TGC/CGC | C->R | missense_variant |
| | GTCAAGCGTCAAAAGTCTGC (SEQ ID NO: 248) | | GTCTGC/ | VC->AR | |
| | GCAGACUUUUGACGCUUGAC (SEQ ID NO: 249) | | GCCCGC | | |
| g69 | GTCTGCCAGCGGCTGAACTG (SEQ ID NO: 164) | TGG | CAG/CGG | Q->R | missense_variant |
| | CAGTTCAGCCGCTGGCAGAC (SEQ ID NO: 250) | | | | |
| | GUCUGCCAGCGGCUGAACUG (SEQ ID NO: 251) | | | | |
| g70 | TCTGCCAGCGGCTGAACTGT (SEQ ID NO: 165) | GGG | CAG/CGG | Q->R | missense_variant |
| | ACAGTTCAGCCGCTGGCAGA (SEQ ID NO: 252) | | | | |
| | UCUGCCAGCGGCUGAACUGU (SEQ ID NO: 253) | | | | |
| g71 | CTGCCAGCGGCTGAACTGTG (SEQ ID NO: 166) | GGG | CAG/CGG | Q->R | missense_variant |
| | CACAGTTCAGCCGCTGGCAG (SEQ ID NO: 254) | | | | |
| | CUGCCAGCGGCUGAACUGUG (SEQ ID NO: 255) | | | | |
| g72 | GCACCCCACAGTTCAGCCGC (SEQ ID NO: 167) | TGG | GTG/GCG | V->A | missense_variant |
| | GCGGCTGAACTGTGGGGTGC (SEQ ID NO: 256) | | TGTGGGGTG/ | CGV->CGA | |
| | GCACCCCACAGUUCAGCCGC (SEQ ID NO: 257) | | TGCGGGGCG | | |
| g74 | AGGAAGGGGCCAAGGCTTAA (SEQ ID NO: 168) | GGG | TTC/TCC | F->S F->P | missense_variant |
| | TTAAGCCTTGGCCCCTTCCT (SEQ ID NO: 258) | | TTC/CCC | | |
| | AGGAAGGGGCCAAGGCUUAA (SEQ ID NO: 259) | | | | |
| g75 | AAGGAAGGGGCCAAGGCTTA (SEQ ID NO: 169) | AGG | TTC/TCC | F->S F->P | missense_variant |
| | TAAGCCTTGGCCCCTTCCTT (SEQ ID NO: 260) | | TTC/CCC | | |
| | AAGGAAGGGGCCAAGGCUUA (SEQ ID NO: 261) | | | | |
| g76 | AGGTGACAAGGAAGGGGCCA (SEQ ID NO: 170) | AGG | GTC/GCC | V->A | missense_variant |
| | TGGCCCCTTCCTTGTCACCT (SEQ ID NO: 262) | | CTTGTC/ | LV->LA | |
| | AGGUGACAAGGAAGGGGCCA (SEQ ID NO: 263) | | CTCGCC | | |
| g77 | GTGTGTAGGTGACAAGGAAG (SEQ ID NO: 171) | GGG | TAC/CAC | Y->H | missense_variant |
| | CTTCCTTGTCACCTACACAC (SEQ ID NO: 264) | | | | |
| | GUGUGUAGGUGACAAGGAAG (SEQ ID NO: 265) | | | | |
| g78 | GGTGTGTAGGTGACAAGGAA (SEQ ID NO: 172) | GGG | TAC/CAC | Y->H | missense_variant |
| | TTCCTTGTCACCTACACACC (SEQ ID NO: 266) | | | | |
| | GGUGUGUAGGUGACAAGGAA (SEQ ID NO: 267) | | | | |
| g81 | GATTGAGCTCTGAGGTGTGT (SEQ ID NO: 173) | AGG | TCA/CCA | S->P | missense_variant |
| | ACACACCTCAGAGCTCAATC (SEQ ID NO: 268) | | | | |
| | GAUUGAGCUCUGAGGUGUGU (SEQ ID NO: 269) | | | | |
| g82 | TAGCAGATGATTGAGCTCTG (SEQ ID NO: 174) | AGG | TGC/CGC | C->R | missense_variant |
| | CAGAGCTCAATCATCTGCTA (SEQ ID NO: 270) | | ATCTGC/ | IC->TR | |
| | UAGCAGAUGAUUGAGCUCUG (SEQ ID NO: 271) | | ACCCGC | | |
| g83 | CAGAGCTCAATCATCTGCTA (SEQ ID NO: 175) | CGG | AGC/GGC | S->G | missense_variant |
| | TAGCAGATGATTGAGCTCTG (SEQ ID NO: 272) | | | | |
| | CAGAGCUCAAUCAUCUGCUA (SEQ ID NO: 273) | | | | |
| g84 | AATCATCTGCTACGGACAAC (SEQ ID NO: 176) | TGG | ATC/GTC | I->V | missense_variant |
| | GTTGTCCGTAGCAGATGATT (SEQ ID NO: 274) | | | | |
| | AAUCAUCUGCUACGGACAAC (SEQ ID NO: 275) | | | | |
| g85 | ATCATCTGCTACGGACAACT (SEQ ID NO: 177) | GGG | ATC/GTC | I->V | missense_variant |
| | AGTTGTCCGTAGCAGATGAT (SEQ ID NO: 276) | | | | |
| | AUCAUCUGCUACGGACAACU (SEQ ID NO: 277) | | | | |
| g88 | GACACATGTCATTTCTGCTG (SEQ ID NO: 178) | TGG | TGT/CGT | C->R | missense_variant |
| | CAGCAGAAATGACATGTGTC (SEQ ID NO: 278) | | ATGTGT/ | MC->TR | |
| | GACACAUGUCAUUUCUGCUG (SEQ ID NO: 279) | | ACGCGT | | |
| g89 | AAATGACATGTGTCACTCTC (SEQ ID NO: 179) | TGG | AAT/AGT | N->S | missense_variant |
| | GAGAGTGACACATGTCATTT (SEQ ID NO: 280) | | AATGAC/ | ND->SG | |
| | AAAUGACAUGUGUCACUCUC (SEQ ID NO: 281) | | AGTGGC | NDM->SGV | |
| | | | AATGACATG/ | | |
| | | | AGTGGCGTG | | |
| g90 | AATGACATGTGTCACTCTCT (SEQ ID NO: 180) | GGG | GAC/GGC | D->G | missense_variant |
| | AGAGAGTGACACATGTCATT (SEQ ID NO: 282) | | GACATG/ | DM->GV | |
| | AAUGACAUGUGUCACUCUCU (SEQ ID NO: 283) | | GGCGTG | | |
| g94 | GTTACCCACCTAAGCAGGTC (SEQ ID NO: 181) | AGG | — | — | splice_region_ |
| | GACCTGCTTAGGTGGGTAAC (SEQ ID NO: 284) | | | | variant |
| | GUUACCCACCUAAGCAGGUC (SEQ ID NO: 285) | | | | |

A representative DNA sequence of CD5 gene is provided by NCBI Gene ID: 921, shown below.

(SEQ ID NO: 182)
GAGTCTTGCTGATGCTCCCGGCTGAATAAACCCCTTCCTTCTTTAACTTGGTGTCTGAGGGGTTTTGTCT
GTGGCTTGTCCTGCTACATTTCTTGGTTCCCTGACCAGGAAGCAAAGTGATTAACGGACAGTTGAGGCAG
CCCCTTAGGCAGCTTAGGCCTGCCTTGTGGAGCATCCCCGCGGGGAACTCTGGCCAGCTTGAGCGACACG
GATCCTCAGAGCGCTCCCAGGTAGGCAATTGCCCCAGTGGAATGCCTCGTCAGAGCAGTGCATGGCAGGC
CCCTGTGGAGGATCAACGCAGTGGCTGAACACAGGGAAGGAACTGGCACTTGGAGTCCGGACAACTGAAA
CTTGGTAAGACTAGTCCTTGGAACTTGCCCACTCCATTTGAGTGGAAGCATGGCCCAATCACCCATGGCA
TGCCTTTATCAGCACTTTGGTTTTGGTTTTGGTTTTGACTTGGTTTGAATCGCTTGACAGGACTGGTCTT
GGGAACTTGCCTACTCCATTTGAGTAGAAGCGTGTCCTGATCACCCACAGTGTGCCTGTACCAGCACTTT
GGTTTTTGTTTTTGACTTGTCTTGGATTGCTTGATACTTTGGTTTTGGTTTTGACCTGGCTTGGATTTCT
TGATACTCTGATTTTGGTTTTGATTCTGGCTTGGCGTAAACTGTAAAAGTGTGTGTGTGCCCTTTTTACC
CGTTCTTTGTTTTGTGGTGTGAGCATGGTGTTTTGTCTCGAGGAAACATGGGTCAGACACAAAGTAAGCC
TACCCCACTAGGAACTATGTTGAAAAATTTCAAAAAGGGATTTAAGGGAGACTCTGGAGTCACTATGACA
CCAGGAAAACTTAGAGCTTTGTGTGAGATAGACTGGCCAGCATTAGAGGTGGGTTGGCCATCGGAAGGAA
GCCTGGACAGGTCCCTTGTCTCGAAGGTATGGCACAGGGTAACCTGTAAGCCAGGGCACCCAGATCAGTT
CTCACATATAGATTCTTGGTTACAGCTAGTTTTGGACCCCCCGCAGTGGTTAAGAGGACAGGCAGCAGCA
GTACTAGTAGCAAAGGGACAGTTAGTTAAGGAAGGTTCTCGATCCCACCCGCCGAGGGAAGTCAGCACCA
AAAGTCCTGTCCAACCCAACACCAGAAGAGTCACAGCAGGAATTGGTACCAGCAGTATCCCCTCCTTATC
AAGAGGAAGGGCTCCCCACTCCTGAGCCCACAGCACCTACAACTCCACCAGATAACCACACCCCAGACCA
CCCAGAGTAGACAAAAGAGGAAGTGAAGCCACGGGAGAAACTCCTCCCTTGAAAGCTCGCTTACAGCCCA
AGACTGGAATCCAAATGCCCCTGAGAGAGCAGTGATATACTGGGGTAGATGAGGACGGACACATGGTGGA
AAGGCGTGCCTTTGTGTATCAACCTTTTACCTCTGCTGACCTCCTCAATTGGAAAAATAATACTCCATCT
TACACTGAAAAGCCTCAAGCTTTAATTGACTTGCTCCAAACTATTATACAGACTCATAATCCTACTTGGG
CTGACTGCCACCAGCTGCTCATGTACCTCTTTAATACAGGTGAAAGGTGAAGGGTGCTCCAGGCGGCAAC
TAAGTGGCTAGAGAAGCACGTCCCAGCCAATTACCAAAACCCCCAAGAATATATAAGAATTCAGCTGCCA
GGAACAGACCCCCAATAGGACCTGAACGAGGGACCAGACATGGAGAGGCTAAGACGGTACCGTGAGGCAT
TAATAGAAGGTCTAAGGAAAGGGGCTCAAAAGGCTACAAATGTAAATAAGGTCTCTGAGGTCATCCAAGG
AAAAGAGGAGAGTCCAGCACAATTCTATGAAAGACTGTGTGAGGCTTACTGTATGTACACTCCTTTTGAT
CCAGATAGCCCTGAGAATCAGCCCATGATTAACATGGCCTTACTTAGTCAAAGCACGGAATATATCAGGA
GAAAATTGCAGAAACAGGCTGGGTTTGCGGGTATGAATACCTCACAGTTACTGGGGTTTGCGGGTATGAA
TACCTCACAGTTACTGGAAATAGTCAATCAAGTGTTTGAGAATGCAACAAGCCACAGAGAAAGCCGTAAG
GAAGGCGAACGCCAGGCTAGGCGAAACGCCGACTTACTGGCCATGGCCATTATGGGAATTCCCCCGAAAG
GAGAGGGAAAGGGGGGTTCTGGGAAGAATACCCAGTCTAATCTCCCATGCTTGCAATGTAACCAATGCGC
CTGTTGTAAGGAAGTAGGGCATTGGAAAGATAAGTGTCCCCAACTGAAGGAAAAGCAAGGTGATTCAGAA
CAAAAGACCTCAGATAAAGATGAGGGAGCTTTGTTCAATCTGGCTGAAGGGCTACTGGACTGAAGGGGAC
CGGGCGCAAGCACCCCCAAGGAGCCCAAGGTCAGGATTACAACTGGGGGCAAGGACGTTAAGTTTTTGGT
TGATAGTGGTGCTGAACCTTCAGTAGTGACCACCCCGGTCACCCCCTCATCCAAGAAAACCATTGATATA
ATCAGAGCAACAGGAGTTTCCACTAAGCAGGCTTTCTGTCTACCACGGACCTGCTCGGTGGGGGGACATG
AGATAGCTCACCAGTTCTTGTACATGCCTGACTGTCCCTTGCCCTTGCTGGGAAGAGACTTGCTTAGCAA
GTTGAGAACCACCATCTCCTTTACAAAGCAGGGCTCTTTACAGCTAAAGTTACCAGCAACAGGAGTTATC
ATGGCCCTTACGGTCCCCCGGGAAGAAGAATGAAGACTTTTTCTAACCAAGCCAGGCCAAGAGATAAAAC
CAGCTCTAGCTAAGCAATGGCCCCAAGTATGGACAGAGGATAATCCTCCAGGACTGGTGGTCAGCCAAGC
CCCTGTACTCATAGAAGTTAAGCCTGGGGCCCAACCAATTAGACAAAAGCAGTATCTGGTTCCCAGAGAA
GCTCTCGAAGGAATCCAGGTTCCTCTCAGGCGCTTGACAGCCTATGGAATTATAGTTCCTTGCCAGTCTC
CACGGAACACCCCCCTCCTACCTGTCCCTAAACCAGGGACCAAGGACTACTGGCCAGTACAGGACTTGGT
CCGAGGAGAAGTTTACAAACCCTCTCCTGAGCCAGTGTTTCAAACTTTCTAAGATGAACTCAATGTGCCA
GTACCACAAATTCCAGGAAAAACAAGAAATTTGTTTTTGCAATTAGCCGAGCACGTAGCCCAGTCTCTCA
ATGTCACTTCATGTTATGTATGCGGAGGAACTGTAATGGGAGGTCAATGGCCATGGGAAGCCCAAGAATT
ACTACCTACAGACCCAGTTCCTGATGAATTCTTGGCTCAAAAGAATCACCCTGATAATTTCTGGGTCCCA
AAAGCCTCAATTATTGGACAATATTGCATAGCTAGAGAAGGAAAAGAATTCACTCACCCCGTAGGACGAC
TTAGTTGTCTGGGACAGAAACTGTATAATGGTACCACAGAAACAGTCACTTGGTGGAGTTCAAATCACAT
AGAGAGGAATCCATTTAGTAAATTCCCAAAGTTGCAAACCGTGTGGACCCACCCGGAGTCCCACCAGGAC
TGGACAGCCCCCACTGGATTATACTGGATATGTGGGCATAGAGCTTACGCCAAATTACCTGACCACTGGG
CAGGTAGTTGTGTTATTGGCACTATTAAACCATCTTTCTTCCAACTGCCCATAAAAACAGGTGAACTCCT
GGGCTTCCCTGTCTATGCTTCCCGCAAAAAGAGAAGCATAGCTATAGAAAATTGGAAAGATGATGAATGG
CCGCCCCCTGAGAGAATCATACAATATTATGGGCCTGTTACTTGGGCACAAGACGGCTTGTGGGGATACC
AGACCCCCATTTACGTGCTCAACCGAATCATACGGTTACAAGCTGCCTTAGAAATAATCACTGATAAAAT
CAGCAGAGCCTTGACTATTCTGGCCTGGCAAGAAACTCAGATGAGAAATGCTATCTCTCAAAATAGATTG
GCTCTCGACTACTTGCTAGCAGCTGAAGGAGGGGTCTATGGGAAATTTAACCTTACTAATTGCTGTCTAC
ACGTAGATGATCAAGGACAAGTAGTTGAAGATATAGTTAGAGATAGGACAAAACTGGCACATGTGCCCGT
GCAAGTGTGGCATGAATTTGATCCTGGGGCCATGTTTGGAAAATGGTTCCCAGCACTAGGAGGATTTAAA
ACTCTTATAATAGGAGTTTTAATAGTAATAGGAACCTGCTTACTGCTCCCTTGTTTGCTACCTGTACTTC
TTCAAATGATAAAAAGCTTCATCGCTACCTTAGTTCACCAAAATGCTTCAGCCCAAGTGTACTATGTGAA
TCACTATTGATCTGTCTTGCAAGAAGACATGGTAGTGAAAATGAAAGTGAGAACTCCCACTCATGAGTGA
GATTCTCAAAAGAGGGGAATAAGGGAGGAGACCACCCCTCATACTGTCTTATGCCCAATTTCTGCCTCCA
AAGAAAGAAGAAGTAAAAACTAAAAGGCAGAAATGAAATCTGCTCGGCACCACACCTGGGCTTGGTAGTT
AAAGATCGACCCCTGACCTAATCTGTTATGTTATCTATAGATTACAGACATTTTATAGAAAAGCACTGTG
AAAATCCCTGTCCTATTCTGTTCCATTCTAATTATCAGTGCATGCAGCCCCCAGTCATGTACCCCCTGCT
TGCTCAATCGATCATGACCCTCTCAAGCAGACTCCCTTAGAGTTGTGAGCCCTTAAAAGGGACAGGAATT
GCTCACTCAGAGAGCTCAGCTCTTGAGACAGGAGTCTTGCTGATGCTCCCGGCTGAATAAACCCCTTCTT
TCTTTAACTTGGTGTCTGAGGGGTTTTGTCTGTGGCTTGTCCTGCTACAGGTCTAAGTGGAATCTAAGTG
GAGGCATCACTGAGCCAGCCTCTCCAGCCCAGGGATGGGGAGGCCCCAGGCCCTAGTGGACCTGGACCAG
TGATCACGTCCTTGAACTACTTCCTACCTGCCTGGGGTGGCCCAGAGTACAGGAAGCCCAAGGAGCCAGG
AGCTTTCTCATCTGGGAGCACAAAGGATGTGAGGAAAAAACGGAACCCAGCCCAGCCCAGGGAAACACAG
GGACTTTCCCCTGCCCCGGGCTTCCCATCTTCTTTGCAGAGACCCTGCCTCTTCTTCAAGGGGGATGATT
CCAGACCCTCTAAGAGCAACAGGGACACGGAGCTCACATACATACCCCACATCAACATGGAGATCACACA
CCTCAACACGGAGATCACACACATCAACATGGAAATCACACACACATATCGAATGGGGATTACACACATC
AACGTGGAGCTCATACATACACCCCAACATGGAGATCACACCCACAACACAGAGATCACACAAGTCAACA
TGGAGATCACACACACACATCAACATGGAAATCACACACACACATCAACATGGAAATCACACACATCAAC
ATGGAGATCACACACACACATCAACATGGAGATTACACATACATCAACATGGAGATCACACACACATCAA
CATGGAGATCACACACACACATCAACATGGAGATCACATTCGCACACATCAACATGGAGATCACACACAC
ACATCAACATGGAGATTCACACATATCAACATGGAGATCACATTCACACACATCAACATGGAGATCACAT
TCACACACATCAACATGGAGATCACAAACACACATCAACATGGAGATCAAATTCACACATCAACATGGAG
ATTACACACACATCAACATGGAGATCACATTCACACACATCTACATGGAGATCACATTCACAACACAAAC
ATGGAGATCACACACACATATATCAAATGGGGATTACACACACATCAACCCCAACATGGAGATCACACAC
ACAGCACAGAGATCACACATGTCAACATGGAGATCATACACACACATCAACATGGAGATCACACACACAT
CAACATGGAGATCACACACACATCAACATGGAGATCACACACACTCGTCAACATGGAGATCACATTCACA
CACATCAACATGGAGATCACACACACACATCAACATGGAGATCACACACACACGACATGGAGATCACACA
CACACATCAACATGGAGATCACTCACACACATCAACATGGAGATCACACACACATCAACATGGAGATCAC
ATTCACACACATCAACATGGAGATCACACACACACAACATGGAGATTACACACACACATCAACATGGAGA
TCACACACACATCAACATGGAGATCACACACACACAACATGGAGATCACACACACACATCAACATGGAGA
TCACACACACATCAACATGGAGATCACACATACATCAACATGGAGATCACACACACACGACATGGAGATC
ACACACACACATCAACATGGAGATCACTCACACACATCAACATGGAGATCACACACACACATCAACATGG
AGATCACATTCACACACATCAACATGGAGATCACACACACACAACATGGAGATTACACACACACATCAAC
ATGGAGATTACACACACAACATGGAGATCACATTCACACATATCAACATGGAGATCACATTCACACACAT
AAACATGGAGATCACACACACACATCAACATGGAGATCACACACACATCAACATGGAGATCACACACACA
ACATGGAGATCACACACACACATCAACATGGAGATTACACACACATCAACATGGAGATCACATTCACACA
CATCAACATGGAGATCACAAACACACATCAACATGGAGATCACACACACACATCAACATGGAGATCACAT
TCACACACATCAACATGGAGATCACAAACACACATCAACATGGAGATCACACACACACATCAACATGGAG
ATTACACACACATCAACATGGAGATCACATTCACACACATCAACATGGAGATCACACACACATCAACATG
GAGATCACACACACATCAACATGGAGATCACACACACACATCAACATGGAGATTACACACACATCAACAT
GGAGATCACATTCACACACATCAACATGGAGATCACACACACACATCAACATGGAGATCACACACACACA
TCAACATGGAGATTACACACACATCAACATGGAGATCACATTCGCACACATCAACATGGAGATCACACAC
ACACAACATGGAGATCACACACACACATCAACATGGAGATTACACACACATCAACATGGAGATCACACAC
TCATCCACATGGAGATCACACACACACATCAACATGGAGATCACACACACATCAACATGGAGATCACATT
CACACACATCAACATGGAGATCACAAACACACATCAACATGGAGATCACACACACACATCAACATGGAGA
TTACACACACATCAACATGGAGATCACATTCACACACATCAACATGGAGATCACACACACACATCAACAT
GGAGATCACACACACACGTCAACATGGAGATCACATTCACACACATCAACATGGAGATCACATTCACACA
CACAAACATGGAGATCACACACACATATATCAAATGGGGATTACACACACATCAACCCCAACATGGAGAT
CACACACACAACACAGAGATCACACAAGTCAACATGGAGATCATACACACACATCAACATGGAAATCACA
AACACACAACATGGAAATCACACACACATATCAACATGGAGATCACACACACACATCAACATGGAGATCA
CACACACATCAACATGGCGATCACAATCACACACATCAACATGGAGATCACACACAACACAGAAATCACA
CACGTCAACATGGAGATTACACACACATCAACATGGAGTTCACACACACATCAACATGGAGATCACACAC
ACTCAACATGGAGCTCACACATACACCTCAACATGGAGATCACACAACACAGAGATGACACACACAACAC
AGAGATCACACAAGTCAACATGGAGATCTCTCTCTCTCTACACACACACACACACACACACACACACACA
CACACACACACTGGCACAAGCCCAGCATTCATGGCGCAGCCATCTCTTGTACTTGCTAGCAAAACACCAC
CTGTGGAAAGTCAGCTCAAACTTCCAACCTCATCCCTCACAAAGGGGGAATGGAGGGTTTGGTTTTGGCT
TTCACTGGAGTCTGCAACAAGAACTGGCATCATGCTGCCCATTTCCCGCCTCTCCCCACCCAGACCCCTG
CCTCAGGGACGCCTGTCCTCAGCCCAGCCCTCAGCTGCAGCCAGGCCTTCAGCCTCCGTAACCCCCGCTC
AGGGTCCCCACCCCCTGCAGCCCTGTCCCTCCAGGATGCATGGCCTTGTCCTGTGTGGGGGTGGCCGAGA
GCACTGCCCCAGCCCTGGGTACCTTGGGCAGGAAGCTGGCAGAGGCCAGGGCTGCCATTCAAACAGGGGC
AGGTGGTTTTGCCAGGAGGAAGTTGACAGTTCAACTTCAAACATGGGTGACGCAGGCCCCACACTGCCTG
CTCCCCGTCCCACCCCTCCCTGAGCACGCCACCCCGCCCTCTCCCTCTCTGAGAGCGAGATACCCGGCCA
GACACCCTCACCTGCGGTGCCCAGCTGCCCAGGCTGAGGCAAGAGAAGGCCAGAAACCATGCCCATGGGG
TCTCTGCAACCGCTGGCCACCTTGTACCTGCTGGGGATGCTGGGTGAGTACCCCTCCCAGGTGTCCTGCG
AACACCCGGGCTCGCTCCAGTGCAAGGAAGGAGTTCCCAGTTTTACCCAAGGCTGACTCTGGGATCCACA
TGTCAGCCCTCTGGAGCGTTGTGGAGATTTGGGGCCACTGGGATCCCTGCCTGCCCCCACTAAGCCGCAG
CTTGGCCCTCTGTCCTGCATGTCCCACCCGCCAGGAGCACAACCTTGCCTCTCTCATGCGCTGTTGAGAA
CCCTGCTTTACCCTTCCAGTGCAAGAGAGACTGCAGGGGGGACCCGCATTTGATGGGGCCCAGACAACTT
GATTCCTAGGCTGAGTTGGATTTTAGCAGAGCATTCAGGCCTCCCTCTGCGAGGTCCCCCACTGACAGCC
CAGCCTTTACTTGGTCGCCTCCAGAGACATGGAAACTCGCCGTCTCCGAGGCAGCTCTGATGATGCTCTG
GACAGACAGTGGGCCGAGGCAGGGTGGAGCCGCCTCTGAGGACGATGTCTGAGCAAATGTCAAGGACTCA
TAGCGGAAGTCCTAGGCCCTCTCATGCCCAGTCCTGGGTCAGTCCCAGAGGGGCCCAGCCAGGCCGGCTG
GGATGAGTACACGCTGGGCCTGCTCTCACTCCAGCCCTGGGTTGTAAGCCCAACCCTGTGTGACCTCGGA
TAGGTCCCCAAGCCTCTCCAGGCCTTGATCTCCCCATTTATGAAATGTGGGGGTTGGGGGTGTGAACTGG
ATGAGGCCTGACCCACTTCCTGAGGATTTAGAAGGTCTGGCAGGGTGACAGAGGTGCGTGACTCTGGGCT
GGAAACAGGAGGATGCGAGCAGCTTCCAGGCTGGCAGGGAGAGGGTCCGGAGGCCTTGCCTGCCTGAGCA
GGGAGCCTCAGCAGCCTTCTAGGGAAACCGGGTGTGCAGGTGTGAGTGTGGGGGAATGCATGTGTGTGTG
TCTGTGTGCATGTGTGTGAGAACTGTGTGTGGGGGAATGTGTGAGTCTATGTGTGTCTGTGTGAGTCTGT
GTGTACGTGTGTGAGAACTGTGTTGGGGGGTAATGTGTGACTCTGTGTGTGAGTCTGTGTGTGAGTCTCT
GGGCATGTGTGTGAGTACTGTGTGTAGGGGAATGTGTGAGTCTGTGTGTGAGTCTGTGTGTGAGTCTCTG
GGCATGTGTGTGAGTACTGTCTGGGGGGGAATGTGTGAGTCTGTGTGTGAGTCTCTGGGCATGTGTGTGA
GTACTGTGTGGGGGGGAATGTGTGAGTCTGTGTGTGAGTCTGTGTGTGAGTCTCTGGGCATGTGTGTGAG
TACTGTGTGTGGGGGGGGAATGTGTGACTCTGTGTGTGAGTCTCTGGGCATGTGTGTGAGTATTTTGTGG
GGGGGAAAGTGTGAGTCTGTGCGTGTGAGTCTGTGTGTGAGTCTGTGTTCATGTGTGTGAGAACTGTGTA
TGGGGGAATGTGTGAGTCTGTGTGTGAGTCTGTGTGTGAGTCTCTGGGCATGTGTGTGAGTACTGTGTGG
GGGGAATGTGTGAGTCTGTGTGTGAGTCTGTGTGTGAGTCTGTGTGTGGGAGTCTGTGCGCATGTGTGTG
AGAACTGTGTGTGTGTGCTTTCATGAGCACAATGCGGCCTCCAAAGGCAGGATTATCTCCCCAACACATG
TGACAATGGCACCTGTGCACACATGTTCACATGGTTCTTCTCACATGTAACAACAACAGGTACAAAATGC
CTTCTGTCCCAAAGTAAGAGGCCTGAGACAGACCTCTGGCTGGCTTCAGGGACCCTGCTAAGGAGGGACC
AGGCCCTTAAACCCCCCACCTGCTCCTCACCTCTGGCCTCAGACTTCATTGAGATAGGGCGGCTCCCAGC
TGAGTATGGGAACCCGTGTCCTGCTCTAGGGCCCTCCCAACTGAATCCCATCATGGGTGAGGCTCCTGAT
GGCACCAGGCAAGTCCCCTGGCCCAACACAGGCCACAGTCAGGAAGACTTGGAGGTAGCCCCTTCACCCC
AGTATGGGGACCTCCTTGAACCCCAGGAATACTCAGTTCCCAGTGTCTGAGTGAGCCGGTGGGTACCAAG
CATTCCCTTTGTTCAGTGGGTAACGGTTTACAAAGCACTTTTAAAGCCATTTTCTGCTGCAAGCTTCACA
ACAGCCAATGAAGTAGGTGGGCCCATTGGCCCCATTTTACAGTGGGGAAAAGCGAGGCCCAGGAAGGCCG
AGTGCAAACCCAGGGTGCAGCAGCTGGTGCACAGCCGAGCTGACCCCAGCCCAGCATTCCTGCCTTCACA
GAGGCCCCTCCCTTAGCTAAAAGGGAAGGCCACCAAAGGCACTTCCGTGTGCAGGAAAATGGCCCTGGGA
CACAACCTCCACCAGCAGAGATACAGCCACCAGAGCTGGGGACACCAAGGGCCAGGGTTGCTGGAGTCAA
ACAGTGCAGGCACCCTTTGAGCCATTCCAAGGAGAGAGGCAGCAATGCCAGAGCCCTGGGGACACCACAG
CCTCCTGGCTGGCATCGGGGCTTGGGGACCTTTGGATCCTGAGGCATGAAGATGCCCCCAGCAGGCCTGG
GTGCTAGTGACAGGCCAGGGCTTCCCACAGTTGCTCCCCTCTCAGCCCCAGCCTGCCTGGACAGCTGCTG
GCAGTGAGGCGGGGAAGCGGCTCTAACACAAACTGCACGTCCCTGACTGGCCTAGCCGATGCGACTCTGA
AAGCCAGCACTGGGACGTGGGAAAGTCATGGACTCTGCAGTTTGAATTCCAGTCTGCCACTTACCAACTG
GGCGACCTTGGACTAGTTCTGTAAGCCTCAGTTTTCTCATCCGTAAACTGGGTGCAACAGTACCTCATAG
AGGCTTATAAGGATTCAATGAATCAATGCACACAGAGCTTAAGTACAATCCTGGCATATAGTATAGTAAG
TACTCAATAAACAATAATAACAGCAACAACTATGATTATTATTATTACCAGGGTCAGCAAGAAACAATGC
GAGGTAGGAGCCAATGGGGAGGAGGGTCTTGCCAGAGGCCTGCACAGCCGTCAATCTCTCTACCCCTCAA
TCCTCTTGCACAGGCTATTTATTCACTAATTCATTCATTTTTCATCTACAAATATTATTGAGCATCTCCT
GTGCATCCAGCTCTGTGCAAAACACTGGGGATACAGGGACCAAGGCAGGAGTCCCTGCCCTCAAGGATTT
GATCAGCTAACAGGAGAGCTGTTAGAGAACACAAGTAAACAAGCACCAAAGACCGGGCATGGTGGCTCAT
GCCTGTAATCCCAGCACTTCGGGAGGCCAAGACGGGTGGATCACCTGAGGTCAGAAGTTCAAGACCAGCC
TGGCCAACATGGTGAAACCTCATCTCTACTAAAAATACAAAAATTAGCCGGGAGTGGTGGCACGCACCTG
TAATGCCAGCTACTCAGGAGGCTGAGGCAGGAGAACCGCTTGAACCTGGGAGGCGGAGGTTGCGGTGAGC
CGAGATCGCGCCATTGCACTCCAGCCTGGGCAACAGAGCAAGACTCCATCTCAAAAAAAAAAAAAAAAAA
AAGGCACCCGAAATATTTACACATTTTGAAATACTGAGTGCAATGAGGAGAACAGCAAGTTCCTCAGGCA
CAATACAGTCTGCACTAGGATAGGAGGACATCCCTTTGGCTGGGCAGGCAGGGAGGGCCTCTCTAAGGAG
GCGAGGTCTCATCTGAGAATCCGAGACCTGAATGAGGGACGGAGCCAGCCAGGCAGAGAAAAAAGGGAAG
AGGGTCCCAGGCAGAGCGGATGGCAGGGCAAAGGCAAGGGGGAAGGACAGGCCTGAGAGGGTGGGGCTCC
TACTGGGAGCAGGGCAGCAGGCCCACTGTCCCAGGCTGGGCGAGGCCCTGGTTGGATCATCCTCCTCGAA
GACCCAGAGCTCCCCACCTCCAGCAGGGAATGAAGGGACCTGGGGGCCCAGGAAGGGACCTGGCCTTCCT
GGGTCACTCTGTCACCATCAGTTCAGAGGGGTTTTCCCTGCAGTGACCAGGCGGCGAATTCAGCTCAACC
TGACAAAATTGATTGAACATCTATTCTGTGTGTGTGTGTGAAATTGAACATCTACTGTGTGTGTGTGTGT
CCATGTGCTGTGTTTGCGTATGAACACATATGTGCACAGCCCTGTGGAGGGGAGCTTTGGAGCAATGCAG
ACCTAGTTTGATTTCTGCTTGGCCACCCACAAGTGAACATCTCTCAGGGTCTCATCTATAAAAATAGGGG
TTCCTTGCTGTGTCCACCTCAAAAGATCACTGCAATAATTACATCGGGTTTATTCAGAGACTAGGTATGG
ATTGAACACCTACTATGTGCCAGATGCTGTTCTAAGTGCTGAGGACACAGCAGTGAACAAAACGGTGGAA
AATCCCTGCCCTGGTGGATCTGGCATAATAGTCAGGGTGTGATGGGGAGACAAACAAGACACAAGCTAAG
CTAGTGATTATGCGAATACTAGACGGTCATGAGAGGTGTGGAGACAGACAGATAGAGGAGGGCTGGGACC
GGAAGCCTTGGTGGGGTGTGAGGTACCTGGGGAGGATTCAGCGGCTCTCATGAGAAGATGATATTTGAGT
CAATATTTGAAAGAAGGGAGTGAGCCCACCGTGTGGGACCTGAGGAGAAGCGTTGCAGGCAGAGGGGCCT
CCAGCTGCAGGTCAGTGGGGGATCGATGAGGCAGCCACGAGGGCCACGGATGCCCCCGTGAGGACTTGGG
CTTTTTCTCCCATGCACTGGACAGCCACACAGGAGCAGAGAGGTGAGCATAACTCTCACCTCTCCAGGTC
CTCAAGTGCATCTTGAACCCCAGGCCAGATGCATTTGGAACTCCAGAAGCTTTCAGATTGTAGAAGGAAA
TACTGTGTACATACTGTGTGCTCTTAACACACGCAGTGAGGCCTGGGGCAGCACCCTGCGTTCAACACAT
TCCCATTTCTGCAGTGAAACATGAATTTGCACACTTAGAGGGATGTAAAGACAGCAAACACCCTCCCACC
AAATGCATTAGAACAAGACTTTTAGATTCTGGATTTTGAATTGCAGATAAGAGACTGTGGGCCTGGATCA
CTTCTTCTATAGCTACCACTATCACCGTGACCAGCATCATCATCACCATCATGTACCTTTCAAGGCCTGA
CTTTCATGGTGTCCAGAGGGACCCTAATACTCCTCTCCGGGGGACAGATACGGTAGGAATAGCGTAGGAA
ATGGGTGGTGGCAGTCTGCAGAGACGACAAGGGTCCCATGCACTGCCACAGATTCCCACAGAGACATCTG
GAATGAACCCCTCCCTCCTTCTGGCCCCAGTTTCCCCCATCTGTACTCCAGAGCAAGCAGGCTCTGGGGC
CTCCCTGGGGTAGACTCCCACAGGCCCTGTCAGGTGATGGAGACAGGGTTTCTCTCCTGACTCTACCACC
AGCTAGCTGTGTGACCTTGGGCAAGTCACTCAGCCTCTCTGAACCTCGGTTTCCTGATCTGAAAATGGAG
GTGAGCATGGTCCCTTCTTCACAGGGCTGTGGGTAGAAAGAACAAGTCTAGTGCATGAGAGAGCTTTGGG
GTAGGTAAAGCTCTAAACAAAACCAAAGCCTTGTCTCTGTGACAGCAGAGACACCGTATATGGTGCGAAA
CTCATGCAACAGCAGACTACTAGTGGGAGAGAGAAATAACAATTCAAACAGCAAAGGTCATTCTGCCCAC
GACCCACTGTCCGCTCACTGGGACAGGCCCTGGGTGAGCCAGATTAGAGACCTGAATCTGCTGTTCCTTC
CTGCGGGCTCCATTCCCAGGCCTCTCCTGGTGTAAGCCCAGTGTGACTCTATATGTGTTTTGCACATAAA
AGCCGCCTAATCATAAACCAATCATTTCATCTGTGCTCAACCCAAGAAAGGGAATTTTGGTCCTTTACGA
CCTTCGTTTCCCTAAAGCCCTGATACAGCTCCAACTCTACAGATGACTGTCGTTTCAGAAAAGAGGGGCT
TCTGACCTTTCAAGTCAGCCAACATATAGGATTCGAGGAAGGAAAATCCAGAATCCTACCCAAAACACAG
GCAGATCTATTGTATTTCATGGACATCGATGGCTGTAGCTATGGGGAGGTCTGTGGACTGTTAGAAGGAA
TCACTGTTTACGGGAAAAAGGGAACACCTGACTTGCAAACTACACTAAATCAGTCATAGGTGTTTTATCT
GTGGAACAAAGTGGTGATAGCACCCCGTCTTCTGAGGCTCTAGAAAGAAATGGGGTCTTCTGTGAAAAAT
CCTTGTCCTGGGGTCTGCACAGGGTAAGGATGCTGTCATTTTATTATCATCGGTCATTATCACAGGCCTG
TGAATCCGGGTGCCTGCCAAACCCTAAGCAGGCCACACCCTTGCCTCCATTTCCCAGGGTGCAGGGGCGG
CAGGACCTAGGAGTGAGCCAGAGGCAGGGGCCAGGAGTGCAGGTTACAAAGCCAGCCGCCCTGGGCTGAA
TTCTGGCCCTGCCGAGTTCTCACTCCCATGCACCACGACAGCCTCCTCCTCTGTAAACTGGGACCCATCA
TGCAAGTCAACAAATATTCATCTGTAATTCCAAAGCCCAAAAAACATTTGTAAGTTTCTGGCAAATGCAT
TTGGTGCAAATCCTGACCTGAACGAATGGGAGGCTATTTATAATCTTGACACATCCCCCTTAGTGTGAAA
CTGCATATGTTTCGCTGCAGACACTGCCATGTGTTTGAAGCCAGGTCTCTCCACACACATGGCGACCGTA
AGGTGCCAGCGAGGCGTGGACGAAACAGAAGTCAAGGGCCACCGCCACTGCCTGGGGCCTCCAGAGATGC
AGCCAGTGGAGCCAGGATGTCCTATTTTTCAAAAACGCCTGGATCTTGAAATGTGTTTAAAAAGCAAAAC
AAAATTAAAAGAAACCTTTTTTTTTTTAATTTGGCTCAAAAAATTTTTAAACACTGTGCAGAGCAAATAA
AACTCATGGGATGCCGTAATGAGAGCCAAAGTGAGAATTATCCTGAATACTGTGGGGGCAAGAGGAATGA
GAGGGACAGGGAGGAGGATATCCTCACCAAGACCTCAGGGCAGGCAGGGCCAAAAATATTGTCTCTGCCA
ATGCAAAGACAAGAGACGGGAGCTCCAGGGGTGTGATAAGCCGTCTGACTTCTTTAGCCCTGAATTTTGC
CTGGGTTTGCCCAGTCCAGGCCCTAAATTGTGGGAGCTGCAAGGGGAGGTGGGCAGGAGAAGGAAGGGGT
CTTCAGAGAATTCTCATCCGTAGCCCCAGTTGCTCGGGGTGAGGGGTGGCTGGCGGATAAGGAAACGGCA
TAGCACTTCCAGGCCTCGGGGGCCTCCTGGTCTGGAGTCTCGGGGATTCTGTCCACAGTTTTGGCAATAG
CAGGAGCCTCAGGCTTCATGAGGCTAGGAAGGATGGTGCAGGAGTAAGCTGTAGAGCCCCCAGAGGGGGT
CAGGGAGACCCTGAACACAGACCGCAGGGCTCAGGGGATGGAGGGTGCTGGTCTGACCCCTGTGAGAAGC
AACTAAAAAATAGAAATAGGTTATGGACCCTCAAAACCTCAATGTTCATTCTGCTCCCTAGATTATAAGT
GAAAAACAAACAAGCAAATAAAAAAAAAACCTGAACATATTCATGGACAGGCTGAAAGAATATACACAAC
ACACTCAGGAAGCTTCCAAGTCAGCTGTACATCTCACCGCCCTCCCTCCACCAGCTGCTGAAATCCATCA
GGAGCTAAAGATTATAGATCACAGGTGTTTGAACCAGGGGGAAGGTGAGGGACTGTGGAATCTAACTTCC
TTACTTTACAGATTAAGAAACTGAGCCCAGAGAGGCTGAGTGGCTTGCCAAGGTCACACAGCACTTCATA
GCTAAGTCTTCTCACTGCATGTTTTACTGACTGAACGGAATCACAAGTTCATTCCCTTTGATCAGGATTG
CACACCAGGGTGGTGCAGGAATCAATGAAACCTGGGGCAGGCCATCATGGGCTTCCTGCTGGAGGGAAGG
GAGGCAGTTTGGGCCTCACTGGCAAAAGCTCAAGACTCAGTGCCAGGCGGCTAGGTTTGAGTTCGGGCCC
CAGCACTCATTAGCTGCTTGGACAAGTCATTTAACCCCTCTCAAGTTTCCTGGTCTGTAAAATCGGGACC
AAAATAATAGCACCTACTTCATAGCACTGTGGTGAGGACTGGGAGAGTTAATGTGTAAAAGGCCCGGAAG
AGTGAAAACTTTTAAAGTGTTGGTGATGGTGACAGTGATGGTGATGATGATGGTGACGGTGATGGTGATG
ATGATGGTGACGGTGATAGAGATGGAAGGAGAGGAGAGGAACTGCTGCTCATCTGGGGACCAGACAGGCA
CCCAGAGGAAGGTTCCAGGGCGACACTCTAGAATGCATAAATATCTTTTTTATTAAAGCATTTGTTAGAG
CATTTTTTAAAGAATTTCACTTGGTTCTCCAGCTTGTTCAGTCTTTCCTGAGAATGTTCTCTGAGCAAGA
CCTAGGCTGTTCTGTGTGGAATCTGCCAAGATTGTCAGGCCCAGTCCCTGTCCCCAGGCCTTCAGGGTCT
GGCTGCACGAGCGCTTCCAGGTCAGACCCAGGTGGTGTTCATCTCTGCATCTCCAAAGCTGGCACTCTGG
AGGAGCAATTGGGTGGCTGGGAGCAGAGAGCATGGGTTTTGGAGTCAGACACATGGTTCGACTCCACCTG
CCAGCCATATTACCCGGGACAGGGCATTTGCCCTCTCCTGACATTTATACCAGGAACGTACTCAACTCTC
GTGATGGGGCTGTGTGGAAGATTAACAACAATATCTGTGATGAACCATACCGTGGGCTAAGTATGCAGCA
GGTGCTCAACAGTTGGCAGCTGCTGTTAATAATGAACGTTGAGTATGATGACTCGGGAAATAGAGATGCT
CCTTGACCTACAGTGGGGTTGCCTCTCAATAAACCCATTGTAAGCTGAAAACATCCTAAGTCAAAAATTC
ATTTAATACCCCGACAAACCCATTGCAAACTTGAAAAGTCGTAAGTTGAACCACCCTAAGTCTGGACCAT
CTGCGCGAAGTTTCCAAGTTAGGAGAAGGAAGTGGTGAGCCCCATGGCTTGGCTCTCTGTGCTCCTGGAC
AGGGAATCTTCCCACTGGGAAAGCTCCCCGAGGGTCTGCTGCCCTGGGCACAGCTTCCCCCAGCCCACCC
TACTCCACAGATCAGTAGCACCAGCTCTGAGCACTAGGCCAGGAAGCACTGGGCTAAGCCCTTTACACCG
ATTAGCTCATCTCATCTTCACAACGGCTCTAGAAAGCTAGTCCCATGCTCATCCTCATGCTATGAGTGAG
GAAATCGTGGCTTGGAGGCACAGAGACAGGACAGACGTGCTCCCCAGACTCTATAGGCAGGAAGTGGCAG
AGCTGATACTCAGAACTGGAGCAGTAGAAAGTCATCGGTGACTGAAAACCCATCCTGAGCTGTGTAGCAC
TGAAGGGCAGATATTTGGACTCCCACTCCCCACAGGTGGCACTTCAAGCTGCCTTAGCCCTCGAGACTTA
GCCAGAGGGATGACTCCTTCAAGGCAGACTGCCCTGCTCCTGATGGGCAGCTGGGATGAGAGGGAGGTGG
GAATGAGAGGTCAGGAGTGAGAGGTGAGGGCAGGACAAAAGGGCCTCCTTCCTGATTTCTCTCGCTAGCT
GGACATCTCCCTGATGGCCTCCTGAGGCTGCCCTGCCCACTGCAATAGATCAGATGATTGGCTGGGCGCG
GTGGCTCATGCTTGTAATCCCAGCACTTTGGGAGGCTGAGGGGGGTGGATCACCTGAGGTCAGGAGTTCA
AGACCAGCCTGGCCAACATGGTGAAACCTCATCTCTACTAAAAGTACAAAAATTAGTCGGGCGTGGTGGT
GCATGTCTGTAGTCCCAGCTACTCGGGAGGTTGAGGCAGGAGAATTGCTTGAACCAGGGAGGCAGTGGTT
ATAGTGAGCCAAGATCATGCCACTGCATTCCAGACTGGGCAAGAGAGGAAGACTCTGTTTCAAAAAAAAA
AGAAAAAATTGATCAGACGATTGACAACTAGACCATCCTTTTCCACCCAGGCCAGAGCTCTGTACACTTC
CCTGTTGGTGAGAATAACCTTCCCCTTCCAAGGCCCGTGACTCCTAGAGCAACAGCCACGAAAGGATCTT
GCAGATTCAGGGGAAACTTATCACATGGCCATGATGACTTCATTACAAAGAGTCTTGATTCGGGGACAAG
GATGAACTGTGGAGCACATCACCCCTTAAAATTCAACCGCCAGCTTGAGAACACTCAAGCAAGAACAGCT
TTTTCAAAGCACACCTATTTTTTAAGAGACGGGGTCTTGCTATGTTGTCCAGGCTGGACTCGAACTCCTT
GGCTCAAGTGATCTTCCTGCCTCAGCCTGCTGAGTAGCTGGGACTACGGCCTTGAGCCATTGGCCCAGCT
AAACACACCTATTTTTTAACACCAAGCAAAAATTTCTCACAAACAATTTGAACACAGCCTTTCACTATTA
ATATATAAAGCAGTCTCCTTGACCCAGTGCCGTAAATCCAAGAGTAAACCCAAGTCGGACAGATGGAAAT
CAAAATACCGTACTTGCACCTAACTGATCACGCTAGGTGGTGAATACAGAGGTTAAGCTTTGAAGCCACG
TGAGCTTGCTACTTCACCTCGTCCCCACCCAAATTAAACCTCTTCCCCAGCCACAGATGAGCTGCTCTGC
TGAGAGCCCCGCAATGGGCAACTTTGCCCCCACTCCACAGTGAGACCAGGCTACAAGGAGGCAGAGCTTC
CACGTGGTCCACATGGGGGAAGGCTGGTTCCCTGCAGACCCGTGTTCCCTGGGCCTGTCGCCTTGGCTCC
CTTGTCAACTAGCTGGCTACACTACTGTGGGCAGGTCACGCCATCTCTTTGAGCTTGGTCTTCTCCAGAA
ATGGGCAGCTGTTAAAGATAAGTGGCTGTCATTCTTATCCTGCCTTGGGAATGCTAGAAAGTAGCAAATG
TTCTCTTGGTTCCCTTTCTTGCTCATTTCTTTCTTTCCTTTTTTTTTTCTTGTAAAGACAGAATCTCACT
CTCTCACCCAGTCTGGAGTATAGTGGTGCAATCTCAGCTCACTGCAGCCTCCACCTCCCAGGTTCAAGCA
ATTCTCCTGCCTCAGCCTCCAGAGGAGCTGGGATTACAGGCATGCATCACCACGCCGAGCTAATTTTTGT
TATTTTTAGTAGAGACAGGGTTTCACCATATTGGCTAGGCCAGTCTCAAACTCCTGACCTCAAGTGATCC
CCCCACCTCGGCCTCCCAAAGTGCTTGGATTATAGGCATGGGCCACCGTGCCCGGCCCCTTTCTGGATCA
TTTCTCCATTCTCTAGAACTAAGGAGCTCAAACTTTTTTGAGTGGAAACATGCGGGACATTTTTACATTG
CAACCCCCAACACACAAAAAGTGATCAGTGCATTCTGACATTCTCTTTTTTATTTATTTAAAACAAAACA
AACAAAAAAAAACAGGGTCTTACTCTGTCACCCAGCTAGAGTGCTGTGGCATAATCATAGCTCACTGCAG
GGTCCAACTTCTGGGCTCAAGCGGTCCTCCCACCTTGGCCCCCTTAAGTGCTGGGATTGCAGTCATAAGC
CACCGTGCACGGCCTCTCTATTCTACTCCATTTCATTTTTAAAAAAATTGATGGCCTGAGTGCTGTGGCT
CACACATGTGAATCCCAGCACTTCAGGAAGCTGATGCACGAGGATCACTTGAGCCCAGGAGTTCAAAACC
AGCCTGGACAACATAGTGAGACCCCATCTCTACATACACACACACATACATGCATACATACATACATACA
CACATACATAGTAAAAAAATTAGCCAGGCATAGTGGCTCATGCCTGTAATCCTAGCTACTCAAGAGGCTG
AGGTGGGAGGATTCTTTGAGCCAGGGAGGTTGAGGCTGCAGTGAGCTGTGATCATGCCACTGCACTCCAG
CCTGGGTGAGGAGAGTGATATCCTGTCTCAAAAAGTAATAATAATAATAATTGATGGTTACATTATTACA
AAGGTTAGCATGAGGGAATTTGAGGTAGGGAGTAATGGAACTGGTGTATACCTTGATTGTGATGGTGGTC
ACACACATCTATATATGTGACATTCACGGAATCATGCAGTAAAGAAAAATCAATTTCACGTCTGTTCATT
TTAAAAGTAACGTTTTTTAAGAAGAAAAAAAATCGATAGTTGCAGCCCACTAGATAGAATTCATATCACT
CAGGGGTTCCAACCTGGAGTATGAAAATTCCTGTCCCTAAAACCCATGATAGTGGATAGGGGGAGGCAGA
AAGGGCCATTGCTCGGGCTGTGGGTGGGTGAGCTGGGGAGAAGGGAGAGAGTGGGAGGTTTCACTTCCTG
ACCCTCCTCTCTTCTTTCTGCAGTCGCTTCCTGCCTCGGACGGCTCAGCTGGTATGACCCAGGTAAGGAA
GAGCCACATGGAGAAAGGCCTGGGGCAGGGGGAGAGTGGGGCTGTGGTTTCATCAGGCCATCGGGGACCT
CTCGATGAAGCCATCACTTCTGCCAGAGTGAACCCCACCCTATAGAGAGAGTGAACCCCAGCATACACAC
AGGCACATAGATGCAGACACTGCACATTAAGATGCTCACATGCAGGTGGGTGCCCTCGACAGCCGTAAAT
CACCCACAAATGCCAGATCTCATGATAATTATTATGACCCGCTCACCATGCACAGAAGACATCCCAGCTC
ATAAATGTACCTTGCAAAGTCTTATTTCCCACCCAATCCTGACAGATGCTCCATGGTCAAAGATGTTTAG
AGCGGAGTCTGCAGAGAGAGGCCGCAGACTGATGGTAAAGTGTGTGGAACGTCCAGCCTTAGACGTTGGA
GTTTAGTCGTAGAGGCTGTTTCCCAAATAGGGTTCCATGGAGCATGTTGGACAAAGGGCAGGCAACACCG
CATCTCCCACTGAAGACTGACAGTGTACACCGGCCCAGTAAAGGATCTGAGAAATCCTGCAGCGAAGAAA
TACATGACACTTTTTAACCCAGCACTTTGCACCCTTATTTCACCACAGACCTCTCCTTCACATTGACTGG
GATAACATCGTGCTGAATGTACCTGGGGAGAATTGTGGCTGAAGGGTCTTGAGATGCCACTGTGCAGCCA
GAGGGGGTGACAGGGGCCTTGGCAGGCCCTAAGCACGATCTGGGCCCTGCACTGACATCCTAGCTCCAAG
CTTTGGCACACTGCTTGACCTCTTGAGCCCGTTTCCCCAGCTGTGAAATGGGAAGAAAATCTCTTGCCTG
GTGAAGCGGCAACGAGCCAGCTCGCTGGGCGGACAGCCTGCAAATCACCCCACCAATGTTAGGTGTTGAG
ACGCGCTCGTTCTCCCTCTAACCAGCCAAGTGCCCTTGGCGAGCCCTGGCCCTGAGCTTTGGCCCCAGAG
TGTCCTCGTCGGAAGAAAAGGGTAGCCTGACCCGTGGTTCCCGACCAGGGAAAACCTCCCCCTCAGAATC
ACCTGGGAGAGCCTTTCACATTTCTAAAAATTTCTTGAATCAGTGACACATTCATCCAGCTCAAAAATTT
AAAAAAATATAAAAAGATCTGTAGTGAAGAGTCTCACTTCCATCTCTATTCCCGTCTATCCCAGTCGCCC
CCGACGTCGACCCCAACACCCCAGGTAATGACTACGTTTATTTGTTTCTTGCATATTCTTCAAGGAGCTC
TTTATGCAAATACACACACACACACCCCACACAGACCCCACACACACACCACACATACACACAACCACAC
CACACACACACACCACCACAAACACCACACACACACCGCACACCACATACACACCCCACACACACCACCA
CACCACACACACACACCACCACACACACACCCCACACCACACACACACACCACACACACCACACACACAC
CACACACACCACATGCACACTACACACCACACACCACACACATACCACACACACACCACACACACCACAC
ACACCCCACACACACCACACACACCACACACACACACTACACACACCACATACACACCACACACACCACA
TGCACACTATACACACCACACACACACCACATACACCACACACACACCACACACACCACGCACCACCACA
CACACACACCACCTGCACACTACACACACCACACACACATACAACACACACAACACACACACCACCACAC
ACACATCACACACACCACACACACTACACCACACAGACACACACACCACACACACACATATATTTTTCCC
CTCATGGAGTTTGATAAAATCACACAAGACACATGTCTACCCATGACCCAGCAATTCCATTCCTAGGTAT
TTACCCAAGAGAAATGGAAACGCCCACACAAAGACCTGTATGTGAATGTTCACAGCAGCCTTATTCATAG
TAGGCAAAACCCGGAAACAAATCAAATGGTGAATGGAAAACAAATTGGCATGTCCGTGTAGTGAACTATC
ATTCGGCAATAAAAAGGAATGAACTACTTATGCGTCCAGTGACATAGCTAAATCTCAAATACATGCTAAG
CGAAAAAAGCCAAATCAAACAAAATAAAATCCATGCTGTATGAGTCCATGTCTATGACATTGTAGCACAG
GCAAAACTCATCTTTTGTGACAAAAAGCAGATCAGTGTTTGCCTGAGGCTAGATTCAGGGAAATTAACTA
TAAAGGGGCCTTAGGGAACTTTCTAGATGATGGAAATGTTCCATATCTAGATTATGGTGGTGGTTACATG
CATGTATACATTTGTCAAATTCCATCGAACAGTATCCTTAAAATGAGTGCATTTCACTGTATGTAAAATT
ACCTCAATAAAATTTTTTTTTTTTGAGATGGAATCTTGATCTGTTACCCAGGCTGGAGTGCAGTGGCGCG
ATCTTGGCTCACTGCAACCTCTGCCTCCCGGGTTCAAGCAATTCTCCTGCCTCAGCCTCCCGAGTAGCTA
GGATTACAGGCGCCTGCCACCACACCCAGCTAATTTTTGGTATTTTTTCTTTTTTTAGTAGAGATGGGGT
TTCACCATGTTGGCCAGGCTGGTCTCAAACTCCTGACCTCAGGTGATCTGCCCGCCTCAGCTTCCCAAAG
TGCTGGGATTACAGGCATGAGCCACCACACCTGGCCTAAAATTAATTTTTAAAGATCTCTTAAAAAGCAG
ACACCAGCCCCAATCTCAGACCCCTTGAGACAGAATTTCCAGGACAGGGGCCATCCTGCTGGACAGTGGG
TGCCGAGAACACCTTGCCCATTTATCTGAGCTCCCTTCTGACTCTGAAATCTGGAGCCCCACCCTCCTGG
GTCTAGCTTCGGGGCTGCCTGGGTCAGGGTCCTCTGGGAAGCCCCTGCAGTGCCCCAGAAGGGACGAAGC
TCACAAGGGGCAAGGCAGGCAGCCCACGGGGCAGGAGGGAGCTCAACTGGGCGTCCTAGGGAGAGGGCAG
TGAGGGGTGCCAGTGGGGAACCCCTCCCAGCCTGACCCCCACCACACCTTTCTGACCCCCAGATTTCCAG
GCAAGGCTCACCCGTTCCAACTCGAAGTGCCAGGGCCAGCTGGAGGTCTACCTCAAGGACGGATGGCACA
TGGTTTGCAGCCAGAGCTGGGGCCGGAGCTCCAAGCAGTGGGAGGACCCCAGTCAAGCGTCAAAAGTCTG
CCAGCGGCTGAACTGTGGGGTGCCCTTAAGCCTTGGCCCCTTCCTTGTCACCTACACACCTCAGAGCTCA
ATCATCTGCTACGGACAACTGGGCTCCTTCTCCAACTGCAGCCACAGCAGAAATGACATGTGTCACTCTC
TGGGCCTGACCTGCTTAGGTGGGTAACTAGCCAGCCACACGGGCACCCTGGGCCTGGGCGCCAGCCCCGA
GGAGACTGCCCGAGGCCTGTGATCTAGGGTCTGAGCAGGCTGGTGGAAGGGGTGGGGGGACCCCAGTTTA
TAACCACTCCCCAAGACACATACCCAGGAGGGGGACTGGAAGGGGCCAGCACCCATCTGTAGGATGGCAA
TGGAGGACCTAGTTCTGCCAATCACTGACTTCATCGTCGCCTCTGAACCTCCATTCTCCCATCTGTGAAG
TGGGGTGGTACTTCCCGCCTCGCAGGAGGCTTAGAGACAACGTGTGGGTCAAGTGGACCTGGTGTGCCAA
GCGGCACTCATGCCAGGAGCTCCTGGTCCTCTCAAGGCTGCTGGCTGCCCCCGGCCCTCCCCACACCACC
CATTCCTCCCTCACCAGAGTGTCTCATTGCAGAACCCCAGAAGACAACACCTCCAACGACAAGGCCCCCG
CCCACCACAACTCCAGAGCCCACAGGTAAGAGGATTCTGAACCCCCCACAGGGAGTCAGAGCTAGCAAAT
AAAAACCCAGGATGCCCAGTTACATTGGAATTTCTGACAAAGGTGGAAATGTTTAGTATTGGTGTGTTCT
ACGCAATATTTGGGACCCCATCACCTCCCAAGGCTAAGCGTTAGTCAGTAGTTGTCCACAAGTTGGGGCC
AAACAGCAAGGAGTGCCCAGGAAGCCCTCGGCGCTCAGGGTGGCTCCCCCTCCTGCTCTCTCCTCTCCTA
GCTCCTCCCAGGCTGCAGCTGGTGGCACAGTCTGGCGGCCAGCACTGTGCCGGCGTGGTGGAGTTCTACA
GCGGCAGCCTGGGGGGTACCATCAGCTATGAGGCCCAGGACAAGACCCAGGACCTGGAGAACTTCCTCTG
CAACAACCTCCAGTGTGGCTCCTTCTTGAAGCATCTGCCAGAGACTGAGGCAGGCAGAGCCCAAGACCCA
GGGGAGCCACGGGAACACCAGCCCTTGCCAATCCAATGGAAGATCCAGAACTCAAGCTGTACCTCCCTGG
AGCATTGCTTCAGGAAAATCAAGCCCCAGAAAAGTGGCCGAGTTCTTGCCCTCCTTTGCTCAGGTAAGTG
AGACCTGGCCAAGCCCCATGACACCTTCTGCTGCCCTAGGTGGGGTCACAGAGCATCCCAGAAGGTCAGG
GAACATGTGTGCAGCACAGGGCACTATGGAGAATACAAGGGAAGTGGAGGCCTGGTCTTGGCCTCTAAGA
GGTAACAAGGGTTGGGGTGGGGAGGATGCATCCACACTCAATGCCTTGGTAATCTCTGCAAAGCTACACA
CCCCAAGCCCAAAGGAACCGCTGGCTCAGCTGCCACATGGGGAAGGGGAGTCGGCAGGACTTCCAGGAGG
AGGCAAGGTCTCTTCGTGGGTCTGGGGGAGAAAAAAAAATGTCTAGGTAGGAATTATGGCTTGTGCCAAA
GATGGGGCACTTGGAGGGGTGGGCCTTGCAGGAAGGGCATTTGTAAATCCCAAGAGTTGGCATTGCAGCA
ATGGTGAGAGGTGGGAGGTCCCTGAGGCCCGGGAGTCAGAGGACAGCAGGCAAAGAGAGAAACAGAGACA
GCCCCCAAGAGGGCTCTGCAGAGGGTCCTGCGCCGTGTGCTGACAAGGTAAAGAGAATGATGCCGCTGTG
TGGTTGAGGGGAGGAGGCAGGTGAGACTGAGTCTGTGAGACCCCGGTGGGCCAAACCAGGATCAATGAAG
AAAACTAAAGGGAAGAAACCACAGCTGAAACGAAGGGAGAAACGGAGAAACTTCGCAGTACGCCTCCCTC
CGGAAGTAGTGGGTTCCCATGGCCGGAAGTGTTCTCAGTGAAGTCCACACTGTACTTACCGAGAGCTTAC
TTCATGCAGGGACCATTCCTGGACATTCTAAATTTTTCACATGGCCTTAGGAGGTGGGTGTTATTATTAT
CATCCCTTTTTACAGTGAGGAGGCTGAGGTTTAGAGAGGCTAAACCTCTCGCCCAGTACTATGGAGCACT
TGCTGGGATTTCTGTTCAAGCAGGGTCACTCTAGAGCCTGTTATCTGAGCCTTGGTGACTCAGTGTGGTC
CTGGAACAGCAGCAGCTGGTTAGAAATGCAGGTGCTCAGGCCCCAGCCTGGACCTTCCGAATCAGAATCC
ACTTTTCAGCAAGGTCCTCAGTGATTTATGTGCACAAATCTAGGAATCACTGCTCGGGGCCACCTGGCTG
TCCTGCAAGCACGCTGGGTACCTGGTGTAAATTAGTAAAATGCTGCCCCTCCCCCAGCAGAAGCAGCTCC
CAAGGGGCTCTTGGCCTGATGAGGAGAGCAGGGGCTGGCTCGCAGTCCCACCGGCCCCTCAGCTACTATC
CTTCCTGCAGGCTACAACCTGTAAAACCATACCCAGGGTTCTTGCCTTTGAAGCTTCTTCTAAATAGATT
TACTAGGGCTTTGAAGGTGGATGTTGCTATGGGAACTGGGCAAAGCTGGTGAGATCACAACTCTCTCTAA
ACTGAGCATGTGTGGGCCTCAGCTACAGACTCCCAGGCCCCACCCTGGCAGGCAGAAATAAATGTGTGCC
TCTGTTGAGCCAGGCCCCACTTGCTGAAAGCCTGGCCCTCAGCGTTCAGTGCCTGGGCAGCACGATCTGT
CCCAACTGTGAACTGCCAGGACTCAGGGCAGTAGGTCTTTCCAACCCCCAGGCCTCAGTTTCCCTCTTCT
GAAAAGTCCTCCTTCTTCCCAAAGTGCAGCGGGATAATGACAAGATGTTTCCAAGTGAGAAGTGGACCTA
GGTGGAGCCTATTTCCCACGCTCTTCTCTTATCTCCACCTGGCATGGAGCCGACTCCCTGCTGGTGGCTG
CCAACAGCCTCCCTGCAGAAAGCGCCTATGCTGCTGTGTCTCAGGATGGTGCTGGCAGCTGCCCAGAGGC
ACCAGAGACAGTGCTGGGTCAGGGGAAGAGCCACAGAGGGTGCCAGAAGGAGGGAAGGGCAGAAAAGAAG
GCTGCCTCTCCTGGTGGTCCCACACAGTGCCTTCCCCTAGCCAGGGCCCCGATTTGCCAGTGGCATGGGG
CCCCAGGAAGCAGCACACAGGCTCAGGTTCACACTTGCACCCTCCTTTCCCATTGCTTCCCCTCTCAGGT
TTCCAGCCCAAGGTGCAGAGCCGTCTGGTGGGGGGCAGCAGCATCTGTGAAGGCACCGTGGAGGTGCGCC
AGGGGGCTCAGTGGGCAGCCCTGTGTGACAGCTCTTCAGCCAGGAGCTCGCTGCGGTGGGAGGAGGTGTG
CCGGGAGCAGCAGTGTGGCAGCGTCAACTCCTATCGAGTGCTGGACGCTGGTGACCCAACATCCCGGGGG
CTCTTCTGTCCCCATCAGAAGCTGTCCCAGTGCCACGAACTTTGGGAGAGAAATTCCTACTGCAAGAAGG
TGTTTGTCACATGTGAGTTGGCCACAGCCCACAGTGGGTGGAAGCAGTTACTACTTTACCTCTAGGACCC
GGTCAGGGTGCATGTCTCTAAAGGGAAGCCCTGGGGAGCTAGACAGAGAGTCCCAGAGACCCAGAAAGGA
TGGAGACAGGGAAATTGGAAGGCTAGGGAGCAAGAGTACTCAGACAGCAATGGGCATGAACAATAATAGG
AGACAGGGCAAGCAACAGGCAGATCACTGTCAGGCAGCTGATGAGTCATAGAACTTCTCCTTCTACCTTC
TACTTTTGCAAGGTGGAAGCCAGAAAATCAAATGGTAGATAGCACATATTTGCTACATGAATGGTAACTA
AACGGGAAGTAAAAGAGAGAAATGGATGGATGGATGGATGGATGGATTGGTGGGTGGGTGGGTGGGTGGG
TGGATGGATGGATGGATGGATGGATGGATGGATGGATGGATCGGTGGGTGGGTGGATGGATGATGGATGG
ATGGATGGATGGATGGATGGATGGATGGATGGATGGATCAGTGGGTGGGTGGATGGATGGATGGTTGGAT
GGATGGATGGATGAGTGGACTGATGGATGGGTGAGTGGATGGATGGATGGATGGATGGACAGAAGGGCGG
ATGGACAGATGGATGGATGGATCAAGAATAGAGGCCCCGCTGGCTATTGGTATGCAATCATATATTGGTT
TCTGATGGTATATGATGGCAAGGTGATCACTAATAACTGGCAAATGGTAGAAAATTAAATAGCTTATAAC
AAGTGTGGCAAATGATAGCTAGATGATCAACTGTTAGATGACAAATGGTGGGTGGGTGAGTGGGTAGCAG
GGAATCCTAGAGATAGATGGAGGGATAGCCAAATAGATCATTGCCTTCCGTGCATGCTGGTGAATTTCTC
AGATGAGCAAGGATGACGGCGGAAGCCAGGCAGCCAGCAGCTTCCCTCCCACAGTTTTTCTCCCCCCAGG
ACTGGGACTGACCTAACTCTTCCTCCTTCCCCAGGCCAGGATCCAAACCCCGCAGGCCTGGCCGCAGGCA
CGGTGGCAAGCATCATCCTGGCCCTGGTGCTCCTGGTGGTGCTGCTGGTCGTGTGCGGCCCCCTTGCCTA
CAAGAAGCTAGTGAAGAAATGTAGGTGTCACGGCCCTGAGTGGCTCCGTTCCCACGTGCAGAGACTGGAG
GGGCTGCACTAGAGTCCTCCGGAGGAGGGGTCATGCCTCCAGAGAGCTGGGCACGCAGGACACCTCTGCT
TCACCACCCAGCCTTCCCCTCTCCTGCCCATTAGCCATTTTCTGCCCCAAGTAACAGAAACACAAAGTAA
ACAAAATTGCAATTGCTGATTTGTCCATCTCTGGAGCCCAGCTGAGACCCACTCCAGGTCAGAGAGAAGG
TGTGGCCATTCCCAAGGCTGTGGCAGGCCCAGAGGGTGTTCAGTACAGCCTCTGATGGTAGGAGACACAC
ATCCCAGGCCTAGCCCCACCACTTACCACTGCCCAAGATGCTGATTGGGACACTTCCTCTCTCTGACCCT
CAGTTTCTTCATCTACCAAATGGGCAATCGCACACCTTGTGAACGCGGGGGGTTGTTCTCCTTGCCTTGC
AGAACTTAAGAGGAAATTATTCAGACAAAGAAGTGAGAGGGGAGAGATTGGGAACAGAGGGCCCAGCAGG
ACAGTGTTAGGTCCTAGTGCCTGCCCAGGTCTAGTTCTAAACGCCTGTGGTCTTTTCTCCTGGGGAGCCG
GGCACCTCTACACCATCCTCGGCCTGCTCCTTCGGCACAGGATGGCTGCAATTCCCTTCCCGAGCAAGTT
AGGAAACTGAGGCCTACGAGAGACTCCAGGGGGCAGAGCCCAGCAGCCCTGCCCTCAGCTGCACCTGCCG
CCACCATCTCCCTCCCAGGCCCAGCCCCATCCCCACCCCTGCCTGCCCCCACCCATACCTGCCCCCACCA
CTCTGATGTGCCTTTCTTGTCTCTTGCCCAGTCCGCCAGAAGAAGCAGCGCCAGTGGATTGGCCCAACGG
GAATGAACCAAAACAGTAAGTGTCCCCGGAGGCAGGAGCCTCCCTCAGGCCCTGGACAGAGGCCATGGAG
GCAGAGTCGAGGCTCTCTGCTGACCACAGACGGAGCCTGTGGCTGCTTGAAGCCTCTGATCTCCACGGTG
CTTGTCTGACATGTGACTGTGGCCAACAGACGTCCCCACACCAGGGAGGGCGGAGGCTTCTTGATGGGCT
GATGGCTGAGCGGTTTCTACCGCTGAAGCCTCAGAGTGTAGAAACATCTCATGGTATCCTTTCTGTCCCT
CGCCCCATCTCTAGGGTCCCCAGCAGCCCTCACCTCCTCCAGCAGTATTCCCCAACACAGTAGCCAGTGG
ATGTTAACAGGCAGCACTCACAAGAGGCCTCCCTGATCAACAGATCTGGAAAACCATGAGTTAGGCACAG
CTAAGCATAGCTATTTCAGGACTTATCAGAACCTTAATGGGCATTTTTGTATTGCCCATCTCCAAAGCAA
GGATCCATTAAGAAGGGCTGTCCAAGTTATTTTCATCATGGAACAACTTACAGGGCCAGTATTAGGACAT
TTTGGTGCAGCCAGCCCAACACCATCCCTGGTCTTCATCCTCCCTCCTTCCAGCCACAGTCAGAGACACC
CTAATGGATGGAGACATGGCGCTCTTCCTGCCTCCCATTCTGCCTTCTTCCCCCTTCCCAAGTCCACAGC
CAAATCCTTCAAGAGGGCTCTGTTGAATTTGTTATTAAGAAAGGCTCTGGAAACTTTCTTAATAACATAC
AGGCCCTCAGCTCCTGGAGGGCAGGGAGCAGATCTGTCCCGTCACCTGTGCTCCAGTCTCCTGCTTGGTA
AATGCTTGATGACCGACTCACGGAAGGCCCCACCCTGCCAGTCAGATTGCTGGGTTACCCAAACCTGCTC
TAAAAGTCTGCTGCTGATCCTGCTAGCATGAACCTCCCAGGAAGAAAGGAGAGGGGGATGCATTGAAGGG
GCTGTGGGTGCCTTTGTCCTCTCCCCGCTAACATCATGGGAATAGGAGATTAAAGTTCTGGGCACCTGCT
GGGGAAGGAGACGGAGGACACAGCCACAAGTCTGACACTGTCTCCTACCATCTGCCCAGTGTCTTTCCAT
CGCAACCACACGGCAACCGTCCGATCCCATGCTGAGAACCCCACAGCCTCCCACGTGGATAACGAATACA
GCCAACCTCCCAGGAACTCCCACCTGTCAGCTTATCCAGGTAAGCACCAGCGGGTGCTCCCAGGCACGCA
GGCAGGGCTGCAGCAGGCCGCCAAGGCAGGTCACGTGATGCCCTTGAAGGTCGGGTGATGGCCCAAAGAC
AGAATGGAGACCCCAGGGTGCAAGGGGATCAAACATCGCAGGATGCAGCAGGGGTACGGAAGGGATATAG
CCTTTGCAGGCCCCAGCTGTCCCGTTTGAGACCACAAACTACTCATCTGGAGAGAAGAATGCACTCAGAA
AGCCATCAACCCCAGCCTTGCTGTGACCTAATGGGCCTGTTTCCCTTCAGGGCCTTGAGGGGCCTGGATG
GTAAATGAGGCCCCTTGGATTCCTCCGGGCTAGAAGAGAGCCATTAAGTGGGCTGCAGGAGCGCTGTACT
AAAGGCTCCCAGACTGGCACTACCAATTAAGCCCCAGGGAGCCTGGAGTTCAGCATCATTAGTGAACGTT
TGGCTGATTTGATCAAAACAGATAAGGGGTTGTCACCGGCTCATAAAGCCCCTGTGATCTTCCAACCCAC
TGTGGGCAGCGGCTCTAGGATCCAAGGGGCTCTGTGGAGTCAAAAACCCTCTGCAAACAGCGTCCTTTCT
TTCCCCAGCTCTGGAAGGGGCTCTGCATCGCTCCTCCATGCAGCCTGACAACTCCTCCGACAGTGACTAT
GATCTGCATGGGGCTCAGAGGCTGTAAAGGTGAGCCCGTCTCCAGCCTGACCCCAGCACCCCAGGGTCCC
CCACAGTCCTGGGGGTGAGCAGCAGCCACTCAATGCCTCCCCTCAGTCCCACCCTGCCCATACATGAATA
GGGGACCCCCAGCATCTTGATTCTTGTAAAGGGAAAGACAAACGTCAGCTGTTTAGGTTATACGAGCTGA
TTTGACAATTAGAGCAGAGCAATCATGAACTTTAACAATCAACTCTTAAACTCCATTCGGAAGGCCACAC
AGGCAGAGTCCCTAAGAGCTCCGTGATGGAGTCACACCCCTCTAGAGCTGCCAACCTGCCAGCGGGCAGA
AGGAGCCCCTGGCTCAGGACAGCAGCCGTCCCCAAGATGGAGCCCAGAGCATGGGTCCTGTCCCCGACCT
CCCTGCATCACCTCTGCGGTGTCTTCCTCTAACACGTTTCCTTCTCACCCCACAGAACTGGGATCCATGA
GCAAAAAGCCGAGAGCCAGACCTGTTTGTCCTGAGAAAACTGTCCGCTCTTCACTTGAAATCATGTCCCT
ATTTCTACCCCGGCCAGAACATGGACAGAGGCCAGAAGCCTTCCGGACAGGCGCTGCTGCCCCGAGTGGC
AGGCCAGCTCACACTCTGCTGCACAACAGCTCGGCCGCCCCTCCACTTGTGGAAGCTGTGGTGGGCAGAG
CCCCAAAACAAGCAGCCTTCCAACTAGAGACTCGGGGGTGTCTGAAGGGGGCCCCCTTTCCCTGCCCGCT
GGGGAGCGGCGTCTCAGTGAAATCGGCTTTCTCCTCAGACTCTGTCCCTGGTAAGGAGTGACAAGGAAGC
TCACAGCTGGGCGAGTGCATTTTGAATAGTTTTTTGTAAGTAGTGCTTTTCCTCCTTCCTGACAAATCGA
GCGCTTTGGCCTCTTCTGTGCAGCATCCACCCCTGCGGATCCCTCTGGGGAGGACAGGAAGGGGACTCCC
GGAGACCTCTGCAGCCGTGGTGGTCAGAGGCTGCTCACCTGAGCACAAAGACAGCTCTGCACATTCACCG
CAGCTGCCAGCCAGGGGTCTGGGTGGGCACCACCCTGACCCACAGCGTCACCCCACTCCCTCTGTCTTAT
GACTCCCCTCCCCAACCCCCTCATCTAAAGACACCTTCCTTTCCACTGGCTGTCAAGCCCACAGGGCACC
AGTGCCACCCAGGGCCCGGCACAAAGGGGCGCCTAGTAAACCTTAACCAACTTGGTTTTTTGCTTCACCC
AGCAATTAAAAGTCCCAAGCTGAGGTAGTTTCAGTCCATCACAGTTCATCTTCTAACCCAAGAGTCAGAG
ATGGGGCTGGTCATGTTCCTTTGGTTTGAATAACTCCCTTGACGAAAACAGACTCCTCTAGTACTTGGAG
ATCTTGGACGTACACCTAATCCCATGGGGCCTCGGCTTCCTTAACTGCAAGTGAGAAGAGGAGGTCTACC
CAGGAGCCTCGGGTCTGATCAAGGGAGAGGCCAGGCGCAGCTCACTGCGGCGGCTCCCTAAGAAGGTGAA
GCAACATGGGAACACATCCTAAGACAGGTCCTTTCTCCACGCCATTTGATGCTGTATCTCCTGGGAGCAC
AGGCATCAATGGTCCAAGCCGCATAATAAGTCTGGAAGAGCAAAAGGGAGTTACTAGGATATGGGGTGGG
CTGCTCCCAGAATCTGCTCAGCTTTCTGCCCCCACCAACACCCTCCAACCAGGCCTTGCCTTCTGAGAGC
CCCCGTGGCCAAGCCCAGGTCACAGATCTTCCCCCGACCATGCTGGGAATCCAGAAACAGGGACCCCATT
TGTCTTCCCATATCTGGTGGAGGTGAGGGGGCTCCTCAAAAGGGAACTGAGAGGCTGCTCTTAGGGAGGG
CAAAGGTTCGGGGGCAGCCAGTGTCTCCCATCAGTGCCTTTTTTAATAAAAGCTCTTTCATCTATAGTTT
GGCCACCATACAGTGGCCTCAAAGCAACCATGGCCTACTTAAAAACCAAACCAAAAATAAAGAGTTTAGT
TGAGGAGAAA

A representative mRNA sequence of CD5 is provided by NCBI Reference Sequence No: NM 014207.4, shown below:

| (SEQ ID NO: 183) | |
|---|---|
| 1 | gagatacccg gccagacacc ctcacctgcg gtgcccagct gcccaggctg aggcaagaga |
| 61 | aggccagaaa ccatgcccat ggggtctctg caaccgctgg ccaccttgta cctgctgggg |
| 121 | atgctggtcg cttcctgcct cggacggctc agctggtatg acccagattt ccaggcaagg |
| 181 | ctcacccgtt ccaactcgaa gtgccagggc cagctggagg tctacctcaa ggacggatgg |
| 241 | cacatggttt gcagccagag ctggggccgg agctccaagc agtgggagga ccccagtcaa |
| 301 | gcgtcaaaag tctgccagcg gctgaactgt ggggtgccct taagccttgg ccccttcctt |
| 361 | gtcacctaca cacctcagag ctcaatcatc tgctacggac aactgggctc cttctccaac |
| 421 | tgcagccaca gcagaaatga catgtgtcac tctctgggcc tgacctgctt agaaccccag |
| 481 | aagacaacac ctccaacgac aaggcccccg cccaccacaa ctccagagcc cacagctcct |
| 541 | cccaggctgc agctggtggc acagtctggc ggccagcact gtgccggcgt ggtggagttc |
| 601 | tacagcggca gcctgggggg taccatcagc tatgaggccc aggacaagac ccaggacctg |
| 661 | gagaacttcc tctgcaacaa cctccagtgt ggctccttct tgaagcatct gccagagact |
| 721 | gaggcaggca gagcccaaga cccaggggag ccacgggaac accagccctt gccaatccaa |
| 781 | tggaagatcc agaactcaag ctgtacctcc ctggagcatt gcttcaggaa aatcaagccc |
| 841 | cagaaaagtg gccgagttct tgccctcctt tgctcaggtt tccagcccaa ggtgcagagc |
| 901 | cgtctggtgg ggggcagcag catctgtgaa ggcaccgtgg aggtgcgcca gggggctcag |
| 961 | tgggcagccc tgtgtgacag ctcttcagcc aggagctcgc tgcggtggga ggaggtgtgc |
| 1021 | cgggagcagc agtgtggcag cgtcaactcc tatcgagtgc tggacgctgg tgacccaaca |
| 1081 | tcccgggggc tcttctgtcc ccatcagaag ctgtcccagt gccacgaact ttgggagaga |
| 1141 | aattcctact gcaagaaggt gtttgtcaca tgccaggatc caaaccccgc aggcctggcc |
| 1201 | gcaggcacgg tggcaagcat catcctggcc ctggtgctcc tggtggtgct gctggtcgtg |
| 1261 | tgcggccccc ttgcctacaa gaagctagtg aagaaattcc gccagaagaa gcagcgccag |
| 1321 | tggattggcc caacgggaat gaaccaaaac atgtctttcc atcgcaacca cacggcaacc |
| 1381 | gtccgatccc atgctgagaa ccccacagcc toccacgtgg ataacgaata cagccaacct |
| 1441 | cccaggaact cccacctgtc agcttatcca gctctggaag gggctctgca tcgctcctcc |
| 1501 | atgcagcctg acaactcctc cgacagtgac tatgatctgc atggggctca gaggctgtaa |
| 1561 | agaactggga tccatgagca aaaagccgag agccagacct gtttgtcctg agaaaactgt |
| 1621 | ccgctcttca cttgaaatca tgtccctatt tctaccccgg ccagaacatg gacagaggcc |
| 1681 | agaagccttc cggacaggcg ctgctgcccc gagtggcagg ccagctcaca ctctgctgca |
| 1741 | caacagctcg gccgcccctc cacttgtgga agctgtggtg ggcagagccc caaaacaagc |
| 1801 | agccttccaa ctagagactc gggggtgtct gaagggggcc ccctttccct gcccgctggg |
| 1861 | gagcggcgtc tcagtgaaat cggctttctc ctcagactct gtccctggta aggagtgaca |
| 1921 | aggaagctca cagctgggcg agtgcatttt gaatagtttt ttgtaagtag tgcttttcct |
| 1981 | ccttcctgac aaatcgagcg ctttggcctc ttctgtgcag catccacccc tgcggatccc |
| 2041 | tctggggagg acaggaaggg gactcccgga gacctctgca gccgtggtgg tcagaggctg |
| 2101 | ctcacctgag cacaaagaca gctctgcaca ttcaccgcag ctgccagcca ggggtctggg |
| 2161 | tgggcaccac cctgacccac agcgtcaccc cactccctct gtcttatgac tcccctcccc |
| 2221 | aaccccctca tctaaagaca ccttcctttc cactggctgt caagcccaca gggcaccagt |
| 2281 | gccacccagg gcccggcaca aaggggcgcc tagtaaacct taaccaactt ggttttttgc |
| 2341 | ttcacccagc aattaaaagt cccaagctga ggtagtttca gtccatcaca gttcatcttc |
| 2401 | taacccaaga gtcagagatg gggctggtca tgttcctttg gtttgaataa ctcccttgac |
| 2461 | gaaaacagac tcctctagta cttggagatc ttggacgtac acctaatccc atggggcctc |
| 2521 | ggcttcctta actgcaagtg agaagaggag gtctacccag gagcctcggg tctgatcaag |
| 2581 | ggagaggcca ggcgcagctc actgcggcgg ctccctaaga aggtgaagca acatgggaac |
| 2641 | acatcctaag acaggtcctt tctccacgcc atttgatgct gtatctcctg ggagcacagg |
| 2701 | catcaatggt ccaagccgca taataagtct ggaagagcaa aagggagtta ctaggatatg |
| 2761 | gggtgggctg ctcccagaat ctgctcagct ttctgccccc accaacaccc tccaaccagg |
| 2821 | ccttgccttc tgagagcccc cgtggccaag cccaggtcac agatcttccc ccgaccatgc |
| 2881 | tgggaatcca gaaacaggga ccccatttgt cttcccatat ctggtggagg tgagggggct |
| 2941 | cctcaaaagg gaactgagag gctgctctta gggagggcaa aggttcgggg gcagccagtg |
| 3001 | tctcccatca gtgccttttt taataaaagc tctttcatct atagtttggc caccatacag |
| 3061 | tggcctcaaa gcaaccatgg cctacttaaa aaccaaacca aaaataaaga gtttagttga |
| 3121 | ggagaaa |

A representative amino acid sequence of CD5 is provided by NCBI Reference Sequence No. NP_055022.2, shown below.

(SEQ ID NO: 184)
MPMGSLQPLATLYLLGMLVASCLGRLSWYDPDFQARLTRSNSKCQ
GQLEVYLKDGWHMVCSQSWGRSSKQWEDPSQASKVCQRLNCGVPL
SLGPFLVTYTPQSSIICYGQLGSFSNCSHSRNDMCHSLGLTCLEP
QKTTPPTTRPPPTTTPEPTAPPRLQLVAQSGGQHCAGVVEFYSGS
LGGTISYEAQDKTQDLENFLCNNLQCGSFLKHLPETEAGRAQDPG
EPREHQPLPIQWKIQNSSCTSLEHCFRKIKPQKSGRVLALLCSGF
QPKVQSRLVGGSSICEGTVEVRQGAQWAALCDSSSARSSLRWEEV
CREQQCGSVNSYRVLDAGDPTSRGLFCPHQKLSQCHELWERNSYC
KKVFVTCQDPNPAGLAAGTVASIILALVLLVVLLVVCGPLAYKKL
VKKFRQKKQRQWIGPTGMNQNMSFHRNHTATVRSHAENPTASHVD
NEYSQPPRNSHLSAYPALEGALHRSSMQPDNSSDSDYDLHGAQRL

A representative DNA sequence of CD47 gene is provided by NCBI Gene ID: 961, shown below.

(SEQ ID NO: 185)
GCAGCCTGGGCAGTGGGTCCTGCCTGTGACGCGCGGCGGCGGTCGGTCCTGCCTGTAACGGCGGCGGCGG
CTGCTGCTCCGGACACCTGCGGCGGCGGCGGCGACCCCGCGGCGGGCGCGGAGATGTGGCCCCTGGTAGC
GGCGCTGTTGCTGGGCTCGGCGTGCTGCGGTGAGTGGCTCCTCGCTCCCAGCCCTGCGGCTGCTGTCGCT
TCGCCCCCGCGGGCGTGTGGGCTGCGCCCCAGCCAGCCCGGCGGCGCCCTGAAGAGGGTGGCCGGGGCGC
AGAACACTCGGGCCCTGAGCGCCCGAAGTGCAGACGTGGGAGGGCCCCACGGGGAATCGGGCGCCCCCCT
TCTTCCTCCCTTCCTTTCCCTGGTCGTCTTCTTCCCCCTGGGTGAGAGCGGGGCTCATCTCCTCCACCCG
GTTCTCCATCTCCAAATGCACACACACAGGAAGACTCCAAACCGCGCACCTCGCGCAAAAATTAACTAGC
AAGAAAGGGCGCGATCAGAGGCAAGGGGCTGCAGCTGCTAGGGATCCCCTTCTTTCGCACCCCTCTCCCT
TCTCAGTGCTTAGACGCATTTGGGGTTGAGGGAGGGAGAGTGGGAGGGCCCGGGCCTCTTGCGATGGAAG
TGCGCATTTTGGCGAAGTCGGTGAGAAGGGGTTTCTGCCGTTTGCCTCCCACATGAACATAGGAGCGAAG
AGGACGTAAAGGACACAATTAAGTTTCATTTTCAACTCAGCATTCAATCAGAAGAATCTTCCGGCCGTGT
AATTTTTGCTGTCGTTTTTAATCCTAAAAATAAGCTTGCTGGAAACTCTTCCTTTCTCGTGACCCTCACG
CCCCATCAGCCACTTGCATACATTCTAAATTGTACGTTGAAGTTTTTCCCACTTTATTTGGGGGAACCGT
TTTAAAAGTAATCTGGTTTTCGCCTGAAATAGGAAGACAGTAACCTCCAGTCAAAACATGTGCAGCAGAA
TGGGATTTGGTGTTTTTCGACGCCAAAGAACCTCCACCCCCCCACCCCCACCCCGAGCTTTTGGAATCCA
TTTCGCTTTTGTAAAACGTGTGCTTCGTCTGTAAAAACTGAGCAAGGAATAGAAACATTCGTAGCTCTAC
AGTGAGTGCCTGTCACACTTCACATCCATAGAGCCTTTTGTGAACTTTTATAAGATTCAGAATTCAGCTT
GAGCACCAGTGACAGCAATTGTTACATTTATTCTAGAATGCTAAATTAATTCGATTTTAAACTGATTATT
AGCCTCGGTGTGCCGTTCCTAAAGGCTTGTGACTTTAGGTATTTCCAAGATGCCCTTAAGTCTCTGCTTA
CCACTTTCCTCCCTCCCCGAGCATCCTGGATGTTGGGACTGTGAATCCAGGTCTCCGTTATCTAAATGGT
TGATGTTAGTGTTTCCTGTCATCACGTTTAGTATGCTTGTTGCCTTTACTATCATTATAGCTAAAATAAT
ACTGCTTTCAGAGATGTGTTGTATAATCGCATAATAATTTCAGAACGCCTTCATATTGAACCAGATATGA
AGTGATACAGTATCATTTATTCAAACTGCCTAAAAAGTAAAAAGTATAAACCATATACTATTTTTAAAAC
AGGTAGGTTAGATTTAAATCACGTAGTTTAGAACTGTTGGAATGGTACTTTGAGTGATAGGATTTATGTT
AGGCCTTCTTTAGTAATAGTTAATATCATGTAATTAGCACTCGTTTACACACAATTTTAATGTGTTCGAA
TCCCTAGAGTCATCGAATTCAGAATTTATAGTATTTTATTTTACTTAGTTAATATTAACCTAAAAAAAAA
GCAAAATACAGGCATTATGCAGTTGAGTTGTGTTAAGTGTGGACTGTAAAACAGGATAGTATTTGTTATA
AAATATGTTTTTGTATGTGTTTAATATATAGCTTCAAAAGGACATGTATGAAGAAAGATGTCTCAGCACA
TAGGTAGTTATAAACCAAGGGTTTGAGAGACTGACTTTAGAATCTAGAATGAGTAAGAAAGTGATGGATC
TGTACATTCATTTGTATTGAAGTTTGATTTTGCTTTCAGTTTCGTTAATTAAGCCCCGGGAATCAGGGAC
CTTTCCTGGCACCCTCTACAGTGTTAGTGGCCTTTGTGGTAAAAGAAGTTATCTCAGATACTCATTTCAT
GCAATTACAGGCAAACTGGAAGGCACCTTAATGGTATGCAAGATGTGAAATGAATTACTATGTTGCATTC
CACTCTGCCCCCACTCATGTACAATTATACTTTGTTTAAAAGTGCATAGTTTCAGTGGTATTTATTAGCT
AGCAAATAAAACTTTAAATAAATAATGATGGTGTTGTGAACTGTCTTTTCAGTGGTTGAATGATTCCTCT
TTGTTCAAAATGAGTTGTTTTTTTTTTTAAATCAGGGTACATATGATTAAATAAAATTTTTTTCCATCGT
CTAAGTCTAGTAGTAATTTGGCTTGTTTAAATAAAAATGTTTTCTCTTAAAGAATATATTATTTTTAATT
ACAAGTTGCCATTTTAAGGAAGTAAATGCCTATTTAAAAGTACGTATTTATGGGGCCGCCCCAGCAGTCT
AGAGGCAGTGTTTTTTAAAGCATTACACTAAATGCCTACCATTAGGACACTTGCTCATGCTGTCATCTGT
GTTTTGGCAGATGTTTTATCTTTGTCACTGAGTTGTTCATGGAGATTTGAAAGGCCAAGTTCTAAAGATG
AGAGATAGATGACTTTCCCCCCAAATTGATACATATTCTAAATCCAAAATAATCACGCATATTCGCAAAA
CTAGATTTGCATTTCATAGTATGAATTCAGCTATAGCTAGTATATAGGCATTGCTTTTAATATAGTAATG
TTCTTTTTGTGGCATAATTTTCAATCACTTTCCCTGTCTTAGGTTTCTGCCTACCTTTTTATACAAGATA
AACATTTCTATTTTCATATCTTATCTCTCAGTCTGATTTTAATAATATCGTCTTGGGTTATCATAAAGTT
ACTTTCACTTTGTATACTAGTGTAGATATTTCTTCTGTGATAAAAAGAACAAGAATAATAGTGTAGGATC
AGTTTTGTTAAACTCATTGTAGTACTAAAGTAGAAAATACAGAGCACCCAGGAAATTTGATTCTGATATA
CCTAGACTAAGACAATAGAATCTGGGTTTTTCCTCTTTACGATTATACAGTGTTGAAACAGAAATCAAGC
TACACCCACCTCTGAATATTTCATTCCAAATCCATGGAGCAGTTGCATAAGACTCAGAATCTGGTCAATC
TCATGGTTTGAGATTCAGATTAGGAGAATTAATGTGCATATTTGAATATATATCACACTTGTAGGTTAAA
AGGTAAAGCAAGGGGTTTGGCTAGTTCAAGGCCTGTCAAAACCACTTTTTTCCATTTCAAGCAAGTATGA
GCCTATACTACACACCAAGCAGTTTGCAAAGCCCTCTTTGGCATATAGACAAATTTCAGACAGTATGTCA
GTATGTCTCTTAATGACTTAAGATGTAGTATATAGACAGTAGGGATATCTTGTTTTTATTCCCTCATGGT
ACTTAGGTGCTTGTTAAGGCCATAATTCTGGATGTTATTGACTTTAAACTGCCCTGCCCTTTTTAATTCT
AAGTTGGCCTCTTCCATTCTTATAAGGCCACTTTTAAATTCATTAGGGGGATTGCGTTGGGTCAGAACTA
AGTACAACTTGTGGACACCCCACTCTACCCCAGACCTTTATTTTAGTGTCCATTGAGGAGAGATCTGACT
TCATCCTTTTTGTACGTAACCCAGTGTAGCTAGAGACTCCAGTTCTTCAAGCCAGACACTAGTTCTAAGG
GGCAAAAGTAGGTAGGATTGCTTTCTGGCATGTGTCTTTACTGATGATCTTTGCTTCTTTACCTAAGAGG
CTGCTGAGACTTCTTACTCCATTCCTGAACATCCATTCCCAGCAGATAATTAACTTCCTACTCTACTGCA
AAAAATAGTGGTGTGTGGTTTGGACTCCCTCTGCTTCCCTCCCACCATTACTAAACTGATCTATAACATC
TTAGATAACCTTCGTCTCATTCTCTTCCGTCATCCAGCTTCAGAAGAAAACCTACTCACTTTTCCAACGA
AAGGACAACCTGTCTACCACCTTTTCACTTCTACCATCTTGGAGACCTGCTCAGTCAGCATAATTCTTCA
GTCTTGCATCTTAGATCTTTCCCTCTCCATTGACTCCTTCCCCGTAGCTTGCACACATGATCAAATTTTA
CCCCATATTAAAAAATAAAACAAAAAACCTCAAGAACCAAAATGTCCTTGCCCTTGTTCCTGCCTGCACA
TTGTCAGTTGTTGCCTTCTCTGCTTTCTTACACTGCCAAATTTATTGTAAAAGAATCACTGCCACTTAAT
ATATTTTAAGTGCTTGATAAAGCCATCTTGTTTCTCTCTCATTTCTCAAACATAGTGTGCATCAGATCTC
ACTAGTCCATTGACAGATGGAAAGATATTCTCCCCAAATATTTAAGCCTCTTTTCTCTATAATTAGCCTA
CCATCTAACTAGCTACACATACTATTTTGGTTCACAATTTTAATTTTCCCCTCTTTATCTCCCCTGCTTC
CCACAACTAACTTCTGTTGATTACTGTAAGTTTTAGGTCTTCTCTCCAGTTGTTCTTTACCTGGACTATT
GCAGTAGTCTGTTAACGGATCTCCCTTTCTCTTTTCCCTTGCTGCCCTCAGTCCTTCCTTTACACTGCTG
TCAATGTTTTTTTTTTAATGAAATTAAATCTGAGTGGTTTACAGTTGTTTATGTTATCAAGTCTAAACTC
AGCATGACATTCAAGGAAGCCCTTTAGAATTTTTCTTATTCATCTTTTCCAACCTCATTTCCTAGAGCTC
ACCTAAATAATTCTCTGCTTTCAGTTCTTCACTTTCTTTATCTGGCTGTACACGTTACTCTGCCTAGAAA
GCCCCTTCTTTCCCATAACATTCTTACTCATCCTTTAAGACCCAGTTTAAACATCACCCTCTAGGAAGAT
CATTCATAGACACATTCTTGTGATTTTTAAGGAACTTTTCTATCCAACCATACAAGTGACTTGAGATTTT
CCATGAAAATATGCAGCTTCATGATTTCATAATCAAGTCTATACAAGTGAGAAGCAGTGGCAGGTTCTCT
TGAAATACAGAAGAAAAATCTATCTTCTCCCACATGATTTTTAGATTTTTCTTCTATGGAATTTATACTT
TAAACTTTTTACATTCACAAGGAAGGTATTGAATCCTACTTTCTGGACCCTGTGTTAGATTCTTAGGATA
CCAAATGAAGACAGTGTCTTACTTTCTGAGCCTGGAGAGATCAGTTAAGTAAAATAGTAATTACACAGTA
GTAGGTACAGTGACAACATTTGAACACAAGGCAATGGGAGCAATAAGGAGGGGAAGAATACAATTGAGTA
ATGGTAGGGAAGGGGAACAAGATTGAACAACTCAACTGTGCTTTAGTAGGAAGAGCAAAAGTTAGCAAAC
TGTAGCTGCCATTTATTGAGTACTTGCTGTATATGTGAAAGGTGATACAAAAACATGTTATCTTATTAAA
TCTTAACAATATCTCTATGAGGTATATACCATCACTATGCACATTTTATAGATGAGGAAACTGAGGAGCA
GAGGTAAGTAACTTGCCTAAGGTTATACAGCTAAGAAGTACATGAGGTGAATCTTAAACTAGGGTCGACC
CCTTAATGTGTGCACTTAACTATTATACACCCTATGTACTCAGTGGGGTAATAGTGTATAACAGTTAAGA
GGCTATTTAGGTTTGAGGAACAGTATGTGCTAAGGCATGGGGATGAGAGTGCATTTGCATAGTCATGGAA
TTGCAATAACGTCATTATGATTGGAGCTTAGTGTGGAAATGAGGGAGTACAGGAGGTGGGGCTGGAAAGG
ATCTTGAAGGACCTCGTTTGTATTCCATGCTAATGTACTTAGAATTTAGCCTGAAAAGGGTTGAGGGTGT
TGTTAAAGTATCTTAGGCAAAGGAGTGCACATTTGCATTTAGAACAGTTATTTTGGGAACTGTAGAAAAT
ACATCAGGGCCAGGATGGAGAAACCTTGAGGATGGAGGCAGGGAGCTGGTTAGAAGGGTATGGCAGTAGT
TCGAGGGGGGAGGATGAGTGTCTAATTCATTCATTCAACAGACATTGGATTTAGGAACTATTAATTAGGT
TGAACCTACTAAGAATTCTCCATGGCTAAGGAGAGGGAGTAATCTAATGTGATTCTTAGGTCCAGGTCTG
GTTCACTGGTAGATGATAGAAAATACTTTAGCAAAGTGATTTAGGCTGAGGGTAAGAAGTGGGGGTACGT
AAGTTTTTGGACATACTGACTTTGAAGTAACTGTATTACATCCAAGTGGAAGTGCACAGCAGACAGTTGG
CTGTCCAACATCTGCAGTTTAGAAGAAAGGCAGTGCATGATTCAGTTTTTTATTTAAAAGTTTGCAGGTG
AAGAAAACCATGGGAGTCAGGGAAAATACTAGGGGGCAGAGGGTACTGAATCTGAAGCCCTTAGGGACAC
TAACATTTAGTGTGAGTCAAGGAATAGATTTCAACAAAGAAAGCAAACTAGGATGGGGTGGACAAAGCAG
GAGGAAATTCGTAATCAGGAGATACCATGGAGGCCAAGAGAGAAGAGCAAGAAAGAGACTGCCCACTGGC
TCAAGAACTGTGGAAAAGTCAAGACAGAAAGTGAACCATGCATATTTGATTTTTCACAAGGGATTGGTGG
TGACTTTGGCAAGAGCTGTAGTAATGAAGAAGTAGGGGTGAATGGGTGGAGGCCACGTAACAGATTTAAG
GAGTGAATTGGAAGTGAGGAAATGCTTATAGATTGTTAATTTGGGATGCTAGGGAGAGAGAAAGTATGAG
GGATTTTTTTTAAGTTGGCAAATATTCAAGCAATTTTTAAAGACACACACAAAAACAACACCTTGTAAAT
GGAATGGGGGATGTGGGAGGCTGATAATCAACTAAGGAAAGTCGTTGAGGAGACTGATGGGTATGGAGGC
AGAAATGGAAACTAGCTTTCAGGTGATGGGATTCATTTCAGAAAAGGAAGCAAAAAAAAAAAAAAGGTGT
GGATAGTTGGGGTTACAGGTAGGTTTATAGGGAAGGAGTATTGGAAGCTTCAGGATTCTCCTCTGAAGGT
TTCAGTTTGTATTAAGAAATAGGGAGAGAGGACTGTTGGAGAGTATGGGATAAAGGGCAGAAGGGAGTAA
TTTAAGAGTTGTTAAAAAGAAGCGAACATTTACATGAAATATGTAAAATGGTAAAGATTGAAGGCCCGGA
GGAGCAGGGACTATGACATTCTTCTGTTTTTGCCTATTAGCAACATGAATTTTCTTAGAAATCTGTAAGA
CCAATTACTCTTCTGCCCATCCATAAGGACGTTGTCCATGCAGAAAAGATAACACTTGTAACCTTGTATT
ATATACTTATCATCGCCCATTTGAGAATTGTAAGCTCCTAAAAGATAATAACTATATTTTTTCATCACTA
TATCCCCACATCCTATCACAATATTTCATCACAGGTAATTCTTGACAAATGTTGATTGCATTTTTAAAAT
TTCTAACCTGAACTTGTGTGCTGTGACCACCATGGATTGAGTCTTCTCTGCCACTACAAAGCTCTTTTCT
AGACTATGATATGAGATGGTTTGGGCTGATAGTCTATATTCACCAATACTTGTACAGTTCCAATGAAGGT
TTCAAGTCTAATACTTTTGGCATTTGATATAAAATCATTTTCCCATTTTATTTGCTAATTTATCTATAAC
TCTGGCATTACTCTTGGTTACATTTGTCTGCTGCTATCTGATTTAGACTGCTATAAGCATATCTTGTTTA
TTCAGAGTTTTCTTATTCATGTTAATTATCTGTGTTTCTTTATTTGCTCTCTACATTTTAAATAGTTTCT
TCACTTTGCTGTTTTATGAGAAGGGAGTAGGCAAAAGGAAAAAACCCCAAATCAGACAGTTTTACTACTT
AATAGTTTTTTAATGCATCTTTATAGAGATTGAAGTTGTAGTTAACAGCTAGAGTGATATTTTTGGCCTG
CCCTTATTTATTAATTACTAGTGCAAAGGTTATTCAAATTGTGGTTTATCCAGGTCAATTTTACTGTTAT
TTTTACTAATAGCATTTATTCTACTTAAATTGCTTCAGCTATAAAATGTTTTTATTGTAACAAATAATGC
AGTATAATTATTGCTTTCTGTATTCCTTTGAAAATTCATTCTCTAAACATATGTTATGAAATGGTGGATT
GTTCACCAACTACTTCTTACTTACTTAATTAAAGCCTTTGCAAAAAGTTTCATAGGATGACTGAGTTCTT
CATTCGTACCTCTTTTTTTTAAGAATAATCTCTTGAAAGTCAAACCATGATTCATACAAATATAAAATGA
ACATGTGTCAAAGATTTTATTTCACTAATTAATTAACAAGCAAACTAGCAAGAAGGCAAAACCCTTTTAA
AAGAAAATTGGAAAAACAGACACATTTATAGGCAATCAAGAATGCCTAAATGAATTTGCTAATAAATGCA
AAATTGGTCAATATCCTCAGGGCAGTAAAATGTAGTCTTTTGGAATCCTCTCTTCCACCAGGTGGAAATC
AATTGCAGAACAAGATTTATTTTTATCTGTATGGACAGCAATCCATTTGAATTACTCTCAGTTTTCTACA
ACTTATGAAACTGTAAAACAGCCCAGTCAAAGTCAGTGAAAGGCACAGGCTTCTATAGAGTCAGATAATT
CCAAAGCGTCTGTTAGACATTGCCCAGCACTTGACTGAAAGGATACCCAGTAGTCTTTGTATCCCTTTCA
AAATCTTCACCATATTTTCTGATACTTTCCTTCCTTTATAATTTGAACATCTTCACCCCTTTATTCTTCT
CTGGTATACTATGTCCTCTCTCCCTCAGTGTTTCTCTCTCAGGGGAAATAATTCACAATTCCAAAGTTTT
AAAGAATGCAACGGAAGTCCAGGTTTTGCCTTGGGCTTCCTAGTATTTGGGTGGCTAGAAATGTAGAAAA
CTGGGAAGAGGTGAGCTGTGGATGCCCACAATATAGGTTCAGACTGCAATTTCCCAGAAATATAACAATT
TGGACTAGTCAAAGAGGGCCCATATCATTACATTAAAATGCCAGATTATCTAGTTTTTATAGTATCACCC
TACATTTTTACAGCATAGTATTGTTTAGAGAGCACTTGCCGCTATGTTTTCCATGTTAATGCTCAACACA
GTTCTGTGAAGTAGCCCGGTGTTTGTTTTTGATCCTCCTACTTAAGAGATCCTCCTATTTTAGAGAGTAA
ACCAAGGCATAGAAAAGTGAGTTGCTTAAGGCAGCATATTAAAAAGGGGCAGAAATAGTATTTTTACTCA
GGTGTTTTGAACATTGTCCAGTACCTATATTCCAGTATGCATCCTTGCATTCAATTCATGGGGTATTTAT
TGAGTAAACATAGTGTTCTTACAATAGAAACATTCTATGCCTCTGATTTTTATTCCAGTTTATGTAGAAG
TAAATCTTAAGTGTGAACTATTAACAAAGTTGATATTTTATTTATATTTGTTAGTAATTTGTGTTTTGTT
TTTGTTTATGTTTTGAGGGGAAGGCCAAGTAGCTACTTAGGTAAAAGAGTTGCTGAGTGGCTGAAGAATA
TGGAAGACAACTACAATTCCTACACATTCTTGTACATTTTAGTTGAACAATGAGTGATTACATTTATTTA
CCCAGTGCCTCTTCTATAAGGCAACAACTGGTAGTTTATCCTATTGAGAAGCGTAGATAGGATGCTTATT
AGCAGTAACTGCTCTTGGTTTCAACTTGCATCTTACTTAGCTTTTTCACCGTTTTGTGGTTTCTTGGAGG
AAGAATACCCATAATATACATTTGGAGACTGTTGTTCTGTAGTGTCAATGAAATGTGGGGGTGGGAAAAT
GTCATTCAAGACTCCCATACAAAATGTCTATTGCTGCCTATATTTTGCTATGGGAAAGTAGCCACAGATA
ATGTTTTTTTTTTCCTCATTAGTATTTTAAGATTTTCCATCCTAGTGGAAAGATATGATTTGATTCATCC
TATTTACTTTGTATATTAAAGTACAGTAGAACCTGCCACTTTTTTTGGAAATGCAGCATAAGGATAAAGA
TAAATTTCATATCAGTTCAGCAAGTTCTATTTAGCAGTGTGTTGAAGTTGAGACTGAATAAAATATTTGG
TTTGGTTTTCTGTTCAAAATTTTACCTTGATAAGGACAATATTTTTCTACATATATCAGTAGGCAGTAAT
GATTACTTCAAAGCTTCCAAAGCCAGATACTACACCTGCATGTTCCAACATAGTTGCTGAATTTATTCCC
AAGATGCATGTAATGTATACTTTGTATTATTGAGAATGAATAAAGAAAAGTCATAATGATGCCTTCCAGC
TGTGCAAGTTAATATTAAAATATAATTTGTTTGCATATTTCACCTAATAGGTCTTCTTCATTGCTATACT
GTTTACTTAAGTGAACAATGGAAATGTTGCTGTTTATCTTAAGGATTTGTAACATGCCTAAGATCTTACA
GTACAGACTTCTATAATTAATGAAACATTTTTCTTTTTCCTTTCCAGGATCAGCTCAGCTACTATTTAAT
AAAACAAAATCTGTAGAATTCACGTTTTGTAATGACACTGTCGTCATTCCATGCTTTGTTACTAATATGG
AGGCACAAAACACTACTGAAGTATACGTAAAGTGGAAATTTAAAGGAAGAGATATTTACACCTTTGATGG
AGCTCTAAACAAGTCCACTGTCCCCACTGACTTTAGTAGTGCAAAAATTGAAGTCTCACAATTACTAAAA
GGAGATGCCTCTTTGAAGATGGATAAGAGTGATGCTGTCTCACACACAGGAAACTACACTTGTGAAGTAA
CAGAATTAACCAGAGAAGGTGAAACGATCATCGAGCTAAAATATCGTGTTGGTAAGACTTCTATGAAAGC
TTCTTTTTTTATTTGTCCTGGTGCAACCTGATCCTCTTTCGAGAGGAGGCCAAATGGGGATAGGTACTCC
TTTGAATCAAAAAGCAGGCTGTTATTAATAAATGTTGATGTAATGTTTAGCAAGTTTAAGATTGTGATTT
CTATCCTATTTGTTAATCTACTCTTCATGGTAAAACACATTTACTATTTAATTTGTATTACTTGTTAATT
TGTATATTGTGTGGTTCTCAAACTTTTGGTCTAAGGACCATTTTATTTGGCTGATTGGTCCTGGGGTAAT
GTACTCTTCATTTCTGTTTGCTGCCATCCAAGTGCTTTCTTTCCTGTGACTAATTTTCAAGTCTCTTTCC
AATTATTCAGCCTTCTGTTTTTTGTGTTTTTTTTTTTTTTTTTTTTTTTTTTTTAACCTTACACTTTGAC
CAAGGGATGACAAGTCCCAGCCATGCTCCCTCTGGAGAGAATAGAGGAGAGTCAGGAGATTAAATCACGT
TATCATGGATGATTTCTTCAGATTTTCTGTGCTTAGCCTTACTTTGGTCTTCTTTTTGTTGGCCAAAATA
TGAGAGAACTAGAGATTCAAATTCATTTATTCAAATTCAGACTTGATGATAGCACCATGATTCTGCCCTT
TTTATCACAAACTTGGGTTGCTGAGGTGTAGAAGGTAGTAAAATTAGCTGAAGTAAATAGTCTCCTCTCC
TTGCTGTTTCCTGCTGTGCTAGCTGAGTTCTTCAACCCCATATGAGTATGTTCTTTCTGTTTTGAAATCT
CTATTTAGAATTACCTGTCATATTACCGATCATGGCTTAACCACATTTTGAGAACCTTAACTCTAGATAG
GTTCAGTTCTGTTCAGTTGGATCAACTATTTGCCTAGCTTTTATAAAATAATCTACTACCAGATTAGGAA
TGGGGTGTTTGGTTTGATGAAATGTTTGCACATTACTTAGGATTGAGAATCAGGAGGTCTGAGTTACAGC
CCCTGTTCAGCAACTTACTATTCACCTGTGTGATCTTGAGAAGGTGCTTAACCGAGTCCAAGTTACCTCA
TGTCCGAAATGGAGATGACAATAGATACTTGCTTTTATGTATTCTACAGTCTAATGAGCATAAACCAAAG
TGTGTTGTAAATGTTAGTTGTTTTCCTGGGATGTTTATTTTATGAAGTAAGGTATCAAGTTGTTGAAGTT
AGCCTAGAGCATTGTGAGAGGGATTTGTGTGCAGTGAGGAATGGGCAAGGGATGGCTCCGAGTAGGCAAG
GAGAATTTGAAAGGTCTTTCTGCTCAGAAATCTCTCTTACATTGTAGCTTTTACTATTGATATTAATTTG
GGCAAAGGAGCTTTTTTTGTTGTTGTTAAGTATACCTTTAATGAAGGGTGTAATCAGTCAGTCATATCCA
CTGCAACTAACTGGTATTAGATCATCTAATACCTAGTTGAAATTTTTCTGAGGTAATGTACAATTTTGTG
GAAATGAAAGAATAAGATATGGGGGAACAGTAACTTTTGGGGGGTAATGCAGGTCACTGAGAGGTAAACT
GGAAAACTCAAAATATGAGATTTGAAAGGCCAGAAGATGAAATGAAGATGGAAGAGAAAGGAATGCCAGG
ATCTGAGATAAAAACAGTTTATTATTTTGCGTGTGATTGAAAGACTGTTTTATCTTTGTTTGCAAAATAC
CCTATAAAAATAAAAACTAGACTTATTTTGGGAGCCATTAAAAGGAGAAAGTATTGTTTTATTATAGAGA
ATGTAGAAAATTCTACCAGAGGTTTGAGACATTTGGCTTTGCATCCATTTTAATTTTTTGAAATAATTAT
TGATTTACAGGACGATTCAAAGATAGAGAGCTTCCATGTGCCCTTCACCCAGTCCCCCCAATGGTTTCCT
CTTATGTGACACAATATGAAAATCAGGAAGTTCACAATGGTGCAGTGTGTGTTAATGGTTCGCTCTCATT
TTATCACGTGTTTATTTGCGTAACTACCTACACAATCAGGACATAGACCTATCCATCATAACAAACATCT
CCCTCATGCTTATTCTTACTAGTCATACCGCCACCTCCCACTGTCCCTAACCTGGCAAGCACCAATTTGT
CCTCCATATCTATAATTTTGTCATTTCAACAATGCTATATAAATGGAATGATACAAGATGTGACCTTTTG
AGAAGGGCTTTTTCACTAAGCACAATGCCCTTGAGATCTATCCAAGTTTTTGCATCTATCAATAGTTCTT
TTTTCCTTACTTTTTTTTTTGCTGAGTAGTAGTCCATAGTATGAATATATCACAGTTTGTTTAACCACTT
ACCTATAGTAGGACATTTTGGTTGTTTCCAAGTTTGGGCTGTTACAAATGAAACTGCTCTGAACAATTGT
GTACAGGTTCTTGTGTAGACACAGTTTTCATTTCTCTGGGATAAATGCTCAGAAGTATGGTTACTGGGCT
GTATGGTAAGTATATGCTTAGTTTTTAAAGAAACTGCCAGACTTTTCCAGAGTGGCTATTTCATTTTACA
TTTCCATCAACAATGTAAGAGTGATCTGGTTTCTCCACATCCTCACCAGTATCTGGTGCCACTATGTTGT
AACCATTTTTAACTGAAAAAAACAACAAACAAACAAAAACCAGTTAAAAAGTTATCTGCACAAAAAGGTT
AAATGGGGCAGGACTCTTACTATGGAGTATAAGTTCTTTTCTAATAAGAATACTTACTTGTCACGGACTT
TGAGGATTTAAGCATTAGAAACCATTTTTACTATGTCGTGATTTTTGCAAATACAGGCATAAATGAGAAA
CAGAATTTGCTCAATAGAGAAGTGAATTCTACTTAATAGAATGCAGATAATAGTGACCATTCTTTGTGCC
TTTTTAAATTTTTTGGAGGACTAGGACTGAATACATGGAAACACTATAAGATATAGTTTTATATACCTCT
GTGCCTATGTATGATATAGTCCATTAGAAGGAGCACTGGACTTGAAATTAGAAAGCTGTTTTCTAGACTT
CACTGTTTAATAGCTTAAGAAAAGTTGGATAAAAAACTCAACCTATCTGTCCCTCAAATTCTTCTTAGTA
CAGTAAAGATGGCATCACCATCATGGAAATGTTTGAGATATAAATAATACCTCTCTTACTTAACAAAATT
TTACTAAGTGCCTACCATGTGCCAGTCACCGTACTAGGCATCACAGATTCTGTGATGAATAAGACATTAT
TGCTGATTTCAAGGAAGTGGTTGCAGTACAAATATGAAGAGTGAAGTGACCTAACATTTATTTGCACATG
TGCCACATACTTTATGTAAGTCGTGTCCTGTTATCCTCTCAAGAACTCTAGGAGATAGATGGAGTTCTTA
CCATTTAACAAATAGGGAAGCAAAAGAGATTCAAGGTATTGTTCAAGGTCAGAGTAATAGCCAAGAATTA
AACCTAGATCTCTTTGGCATCAAAACCCAGTATTTTTACCACCGTAATATGTGGTTCATGTGAAAACACC
TTGTAAATAACTAAATACTGAATGCAAATACTAGTAATCATAAAATTTATCATTAATATTTCTGTTCTAT
TAGCAATACAAAAACTTGAAAACTTAAAGTTTATTTTTCTCTAAGCTATTAGAGTTTTGTTTAGAAAGGT
CAGTTCAAGTTCCGCAGTGTTGCCAATTCCATTATTATCAACAGGACATATGGTCTGTTTATAATGAAGA
CATGAAGGCATTGCAAGATCTTTGTATTAGTTTTCTAGTGCTGCATAACAACTACAAATATAGCAGCTTA
AAACACTACCCATTTTTTAGCTCAAAGTTTTGTAGGTGGCAAGCCTGGTCATGGCATGACTGGGTTCTCT
GATCAAAGTCTTAAAAGACTAAAATCAAGGTGTTGCCCAGGGCATACTTTATTTGGAGTTTGGGGTCTTG
TTCTAGGCTCACATAATTGTGACAGAATTCAATTCCTTGTGATTGTAAGACTGGGGTCCCTGTTTCTTTG
CTAAGTATCTGCCAAGGAATTGTAGCAGCTCCTAGAGATTGCCCTCATTTCTTGCCCCATGGCCCCTTGC
ATATTTAAAGCCAGCAAAGGAGAATCTTCCTCTTGTTGAATCCCTCTCACATATTGAGTTTATTTTGCCA
GGAAGAACCCAGACCCTTTTAAGAGACCACTTGGTTAGGTCAGCTCTCTCTCCCTCAAAAATAATCTCTC
TTTCTTAAAGTCAGCTGATTTGGGATCTTAATTACATCAGCAAACTCCCTTTTGCTGGGTGATATAATCA
TGAAAATGAAATCCAATACATTCACAGTCCTAGTCTCCCACACACAAGGGGAGGGAATTATAAAAGATGC
ATGGCTAGGGCAGGGGTTTTGGGACCACCTTAGAATTCTGGGTTTGATTAAATAAATAATGGGACTGTAG
CCTAGCAAGTCGACACATCAGAAAAGCCATCACAATCCTGAAACATCAAGTAAAAATTTGGTTTGCATTT
TAGGATTGTAAATGTTACTGTGGTGTATGCGTGTGTGTGTGTGTGTAACCTGGTGTTCCCTATGTTTGTG
GGAGTTTGAAGGAGACACTTTGTTGATAGGAATGGTGTCTTCACTTTTTTAGGTTGTCTCATTTTTGTAC
AGTGATAAGACAAATGAGGTCCTGGGTTTTAATAACACTTCAGCTTGAAAGCAAAAATTAAACACTTATT
CATTGACTACACCCTTGTAATATCACTTCTTTGCCTTTCCACTAGCAAGAAGTTCATTTTCGTGGAAGCC
ATTCTGCCTGTCCAGGAATTGGAGGAGAGTGATAGACACAGTTGTCAGCCATAGCTTGGGTAGAATAAGG
ATGTGAATGTCCTTGGCTTATCTTTATTAATCTTGTGATGGAAAAATATCTGACATTGTTCTTAGTCCAT
TTTAAGCTTAATTTATGTTCTTAGTGGCATAGAAATTCAGAGCTGAAAGAAGTATCATGTCTCACTCTCC
CCTTGAGGAACAAGAGTTAACGTCATCTGACAGTACTACCATAACAAATCAATAGCTTAATAGGCATATA
AGTGGCTTTATATAAAATGTTGGTTTTTTTCCCCCAGCATCTCAGTTGGTTCTTAAATATCTAATTCCAT
GATCCTCAAACTTTTCCCACTGTAACAAATTAGAGAGAGGAGGAACATGTTCATCAGTGACTGTAGTTCA
AATACAGCAGAAATGTGCAGCAGTGATTTCAAACTGAGAGAATCCTGGATGCCCTTCTCCATTGTGTGCC
CCCCCCCCCCACCCGCCCCACATATCAGGGAACAATTTAAAATCCTGGCACAATAATGAGAAGGGAGAGT
GACAAACTGATAAGTTCCAGTTAAGAATCACTTAGACAGGCCAGGTGTGGCAGCCCACGCCTGTAATACC
AGCACTTTGGGAGGCTGAGGCAGGTGGATCACCTGAGGTCAGGAATTTGAGACCAGCCTGGCCAACATGA
TAAAACCCTGTCTCTACCAAAATTACAAAATTTAGCTGGGCATGGTGGCGCCTGCCTATAATCCCAGCTA
CTTGGGAGGCTGAGACAGGAGAATTGCTTGAACCCGGGAGGTGGAGGTTGCAGTGAGCTGAGACCACACC
ACTGCACTCCAGCCTGGGCAACAGAGTGAGACTCCATCTCAAAAATAAAAAAAAAAGAATCACTTAGACA
GTATTTTTTGTTCTGTAATCTTCCTCTCTGTCATTGAAATGTCTCATATTTCTTGTTTCACATAGAATCT
GGGAGTCATCAAGTAGTCATCTAGTCTATCTCATCATCTAATTTACGAATTATTTCTATCACACCCCAAG
TAGCTGGCGATTCAGATTTTTTACTGAAAGCTTGACATGATGAGACATTATTACTTCCCAGAGGATCTTG
GTCTGTTATCAGGCAATTTCAGTTCTTCCTTAAATCAAGCTAAAACTTGCTACTATTCTCACTCACTGGG
AGGGCTGTAGTATTTCTCTCAAGTTTATACCTCTCTGTGATGAAAATCTCTGTTCTTTTAGCCATTGCTC
ATGGTTTCGGGATCCCCATTCTAGTCATCTTCCTTTGGAGCAATTCCCTGTTAGATCTTCAAATGTGATG
AGTAAAACTGAGCTCAGGACTGCGTTTGTTGGTCACACCCTTCTCCCTTCCAAATGACTGCTGCTGTAAC
TTTCTGACAGGTCCTGCTTCCCTGTAATCCATTCTCCATGTGGCTGCCAAAGAAGATTTGAAATAAGTCA
GGTTATTTTTCTTCCCTATTTAAAGCCTTCCAGCCGCTTGTCATTGCTCTTAGAATAAAATCCAAACTCT
CCCAACAAGCTTTTAAGACTCTACATGGTCTTGCCCCAGCAAACTCTTCCCATTTGATCTCATCACTGAA
TTCCAGTCACAATGGCCATCCTTTGGTTTCTCAAAGTTGCCGTTCCTTTGGCATCTCTTCCTCCCTGTCT
TCACAATGTAGGCTCCTTCCCATCCTAGGCTTAGTTTCGTTCCTCTCCCTAGAGGCCTTTCCTTACTGCC
TCAGCTTCTCCCACTCTGCACACTCAGGGTCCTCTGCATTGTTCACTGCTGGCCTTCAAGAGCCTAGAGG
AGTTCCTCCCCATGGTGGGCTTTCAATAAGTGTTTGTGGAATAAGTGAAAAATGAGTGGTCACACCAGGA
TCGAATGCACCTCTTACTTTTGTTAATATATTCAAATTAATATTAATATATTAATATTCTATTAATATAT
CCTAAAATTTTGTTGTGTATAGGGGCAGGGACCAATGAAAGAACATTTCATTGTCAGCTCATCTTGACTA
TGAGATCAGATATTGGCTATATTTTGAAGGTAGAGTCAGCAGGATTTGCTGTTTAACTAGAGTGGGATGT
GGGAGAAATCAGGGAGTCAGGTATAACTCCAAGGTTTTCAGCCTCAAGCAACTGAAGAAATGGGGTCACC
ACGACTGAAATGAGGAAAATGAGAGGGGGAGTTTAGTGGGGGATGGGGTGATGAAAATATCAGGATTCAT
TGTGGAACATGAACAGTTAAGATTAACTGAAGAGCCTGAATTCACTGTTCCTCAGTTTCTACAACTATAA
GGAGGGGTTAATAATTTCTCACATCATAATTGTGAGGATTTGAGGAGTTGAGGTACACAATTAAAAACAA
AAGCACAGGGGAAGTAAGGAAAAATACATCAATTAAGCATGGCTATTAGACCATACAGCCTTACCAAATC
CATTGGGGATTAGATACAAAAATCCAAGCATGATCTCCAAAATTTTTTCCTGGTAGGGTTTTAGGCTATA
CTGAAGTGACTTTTCTTTGGCATAAGAAGATATTCAGTTATACAGTTGGAAATAAAGGTATTGATTTGGA
GTATCCAAAAACATCTCTCAGTAGAGATCCACAACCAAAGAAGCATAAAAAAAAGTCTTCCATTCACTCG
CAAAACTGTCTTATGACCATTGCAACCCTCAACAGCAAACATATGGAGTCTCTACTACATATAGAGGAAA
ATGCTAGATTCTGAAAAGCTAATATCTATAAAACAGAGTTTATGATGTTGTTATATTCTGGGGATTGATG
TAAGCATTTTACCAGGACTTATTATAGCATTATAAGCTTAACACAAGAAATATGTGGCTATCATTATGGA
GTGAAAGGCTGGAAAATTCCTCACCTGTGACCTGAGAGATAGTGCGTGGTAGAATTTGAAAGAAGTGTTG
ATTTCAGAGGAATGCTAGTGCTTGCTTAGGGCAGTATTCAGAAGAACTCTTCCAATCACACAGCCCCTTG
TACAGGCAAACTCCGGAATACTTTCAGTACATTTACTTGGCTTTATTGTTAGAGCAGAAACTTCACAGCT
AAAATTATGGTCTTAGGGGTTTATAAGATGCAATTAAACTTAATTTTTAAAAGTTCTCACAAATCTTATT
TGAAGTCTTAATATCTTAATTTCCTTTATATAGGATATGTGGATATATTTTTATGATAAAAGTAACATGT
GACCAACTTAATAACAGCTTCGTTAATATCCTAAAGCCACTCATCTTACTTTACATATAAACAGTCCTTC
TGAAGCCTAATTATGCATGGATAATTGCAGAGTGAGTTTGGGAAAAGACCAACCGGGCACATCTCTCTCC
ATTTTAACCACAATGAGAAGAGAGTACCTAGTGGTGCATCTTCCTCGCTTAGGTTCCCTGAGTCTGTCTT
TACAGGAAGACTTCATTGTTACTTGAAGGTACATTCTTGGAACTTTACACACCCAGCCACTCAACACATG
AATACATACTTATTTTAGTTAACTGAGTACTTAGTGAGTGCTAAGCTTTGTTTTAAGCCCTTTGCATGTA
GTTTATTAACTTTGTTAATAGTTACAACAGTCCTTCAAGATACATGTACTGTGTTATACTAGTGTAACAG
TTGAGGACACCAAAGAACAAAGAGGTGTTGACACGTGGTCACGGTTCCATAGAGTGTTAAGTTAGAGTTG
GGTTCAAACCCTGGCAGTGTGGCCACAGAGCCTTGTTCTCAGCCACTGCACTGCACTACCTCCTCCCGTG
AAACATAAGAAAAATGTGAGAAATGCTTAAGTAAGTGTAGTTTTTATTCATAAATAAAATTTACATAAGT
ACATTATGTGTAATTTGTTTTATGTATATGTGTGTATGTATAAATAAATACATGTAAAAATAAGGCCACA
GTTTTAATTTTTTTCCATCTCTATAATAAAGCATGTATTATAGACCATTAGCAGAATTTAAAGTGTTATA
GTAAATATTAATTGTGACTTTTGTTTTCTTCTTCCCCAGTTTCATGGTTTTCTCCAAATGAAAATATTCT
TATTGTTATTTTCCCAATTTTTGCTATACTCCTGTTCTGGGGACAGTTTGGTATTAAAAGTAAGTATTAT
TTCTACTTTTCATTTATGTTTCAGTGATGATATAGTTATTTCTACGAGACATTGTCAGCGAAATATTTAA
AGTTGTACTAGGAAAAGTGCTATTATGATAAATATGAGTATGTAATTTGAATACTACTAGTCTCCTTGAA
GTATATGTTGTCGCCCACATTTTGCTGCAGTTCACTTTTAATTCCTAAGAAGGTTGTTTTCACTTGGTGT
TTTTTTAATCTCTTAAGAATGAATAGTAGGAATATTAGTACCAACACCTTAAACTCATGTCACATTTTAA
TATTCACAGAACATCTACACACACATTATGTTATTAGGTAAACAGGTGGTGACAGCCTGCATTAGTTTTA
AGGTAGGACGTTATACTTTGGAGCATTTAGATTCCCCTCTTTTTATTTTCCCAGTTTGATTTTCTCTGTG
TACACGTGTTCACCCTTGGAAAAGTCCAGTCGGAACTATGTTTTGTCATCCTCTGCGTGCAGTTCTGCAG
CCTCTAAAGAAGCAGCCACCAGAGAGTTAGGTTCTTTGATCTTGCTTTCCTATAATAGTAACGTAACCAG
ACTTCTGAAGGCAGATCTTGATGCTGCATTAGATTTAGCTTCAACAACACAGAATTGTCATTACTAGGCA
AATAGGTAATATGCATTACGGTTAATGTTTAATCAACCATATTTTCATATTTTGGTAAAGAAAATTTACA
AAATTAATGAAGTTCTGAGGTGACAGTCTAAACTTTTAAGCTTTTTAAATACAAGATTTATTCCTTCTTT
CCTTGTAGCATCCTGAAGACCAGTAAACATTTATATAAGCAAGGAATAAATACTGCTTATTTAATTTATT
CTGCAACCTTTAAACACACAAAAGCTAGTAAACTATTGCAGTGGATTGCCCTGTTGTATATTTTATGAAT
TTTACTTTTACTCAGCAGTTTAAGCTGTCTATATCTATGGTGGTGTATAAACATGGAAGGGAGATGACTG
ATTGATTATATGTTTTAAGCGCTTTTCTCAGTGTATGGCATTCTGGAAATGCTTAGTGATTTCAGAAATG
TTCTCAACTTTTGTCTGAAAGGAAAAAAGGGGGAAGAAAGGGGTGGCAGTGGCAACTGTCAAGACATTTT
ATAACTTTTACTTTCAAGATAGTGTCTAGACTTCTTTTGGAAATTTTCTTATAATCTCTTAGTTTTTTAT
GTCAAGAAAAGGACTGGTGTAGCATTAAGACCTATTCTGGCATCAATGATATTAGGGAAAGCTTTTAACC
ATATTGGCAGAGCCAGACTTTAAGGGCTAAGTCATATGACTTGGGCAGGGAACAGCCTTTTTCAAAGATC
ATGAAATTATTTCACATAGTGCGATTTTTTGAGTTTGGCTTGATGGATGTTTGCTGATCCAGACGTTATC
ATTGGTGACATTATTTTTAGTGAAATGAACCAGGAGTGAGTTTGTTCACTGTTGGCTGTATTTTTACAAA
ATGAGCTTTACAATATTTTTTCTGACTTAAAAAAGTCATACATATCACTTTAGAACACCTCGTACAAAGT
AGGGAGTTATTTAAAAAAAAAAAAAAAAAACTCATCTGTAGTCCCCAAACCTAGAAATAAACTATTGGTA
TATTCTGGTGAATTTCCTGTTTCCCTTTCTGTCTTTTTCTATTCATTTTTGTTATCTTTTTTTTAAAAAA
AGGTAACCATAAATGGACTCGTACAGCTTATATGCTTGTACGTTCTGATGTTCCCCCATGTCTTCAGAAA
TCTTGTTATAGCTGCCTGTGTTCTACTTTTGTGGATGCACTATAATTTACTTAACTCTCATCTTGATGGA
TTTTAAGGATGTTTCCAATTTTTTTGATACTATGAAAATAACTCTGCTAAAGATATGAAGCCTGGAATTG
GCAGAGATATTTTAAGACTCTAGATACATACTGGCAAAATGTTTTCCAGAAACGTTTGTATCAACTACTA
TTGCAGATGAGAATACCTGTCTTTCACCTCAGTTATAATTCTGATATTATAACGCTAAAATCTCTGCAAA
TTTGATGGGTGAAAAAGCATCTTATTGTAATTGTAATTTTTGTGTTAGTAAGGTGGAATGTTCTTTTTTA
TATTTTTTGTACTGGTCATATTTTAAGGAACGTGCTTATAAACCTAAAGAAATATTTTGGTGGGAATTTT
TTGTTTTGGCTCATCTTGAAACAGGTAGATGTGTGTGTATGTGCATGGAAGAGGTATGTCTATACATGAT
TCACCCAGCCTGCGCTCACATTTAAAGGTGTTGATGATAATAGTAGCTAACCATTTGTGGAGCTCTTGCT
CTGCTTGACAGGTTCTGTGCAAAGTACTCTATATCTGAAATGACATTTATTTCTCCCAGAAACTCTATGG
GCATAGACACTGTTGTTATTCCCGTTTTGTAGATAAGAAACAGGCACAGATAGATTAGGCAATTTGCCCG
CAATCACCCAGCCGTTTCCTGATAGTACTGGGATTTGAACTAGTACTGTTTACCACTGCACTGTACTGCC
TCCCCGTTTTGCATTTATTTTGAGGATTTTATTTCCATGAAGGGTGAACCTATATCTAAGCACATAATAC
TGAGTAGCTAAAACTTATTAGGAGAGCAGAATGTTGACCTGATTTGTTTACTTATTCTGCAAATACCTAG
TGACAGGGTGCCTACTGATTGCTGGACACCAAGCTACATGCCTGAAATGTGGGTGTGATGTGCATATTGC
CCTGGTTTTGTAGCACCCACATTTAAGCCAGGGAGACACGAATGTATGTGCATCCCATGCCTTGTCACAT
CTTTGAGATAGTCATGGATCCAAATCTAACTTTTCTGTTAGTGCCTCTGTTGATGGCCTGAGCATTCCAC
CAAATAGAAATAGAAAGCACCTCTATTCTCCACCTGCTTAGATGTATATTTTTTAGAATCCAGTATGGTA
AATCTTCTAAAGCATTCATAATAACTCAGACCCATGACTTTTATTTTATAGATTTCTAGTTCTGCTTAAC
TTTTCCTGATTACCATTTATGGTCTGTACTTGGCTACAGGGGATTTCTCTCAGTGGTTTTTCCAGCTCTG
CATGTGAGTCTTTGTGGTCCAAGACAAACCTGGACCTTTACCAGGCTAAACTTTCAGTAAAGACAGCAGG
TTATGCCCTCATTTGCTCACTCTGAGGAGAAAGAGTTTCTTTATACCAGAGCTGTATCTTGAAAGATGTC
TCAGGATGCCATTGGTCCTACTGAGGAAGAAGCCTGAGAAGACTCTTAAACTCCCAGAGCCCAGCCAGCA
CTTGGTGAGCCCTGGACCACGTTTCAAAGATAAAGGCCTCTTACAGGGAAATGTTCCTAAATACCTTCAC
CTTTAGCTTAGCTTTAACTTAGGAACTTTTAAGCAGAATCTCTATGCTTAGCAAACAGCTCAGAGATTGC
TAGAATCAAACACCAAGGCTTAACTGATAGTATTGAATTTCAGACGCTTTGTTTGTGTTCTGAAAACTAC
ACCAACTCACAGTTTGCCACTCTACTGACAATTAAGTTCCTGGCTGATTTTCAGGATTCTTCTTTCTCAC
TCTGATATCATTTTAAGTGCTGTCCACCTAGTCCTCCAGTTCCTGCAGGATTAAGGTCCAACTGTATGTA
AAGAACTGGCTAACATTTTGAAATTCTTTGAGATAGGCCTATCTGTTCTTTCTTTGCCTTTGTAACTTTG
TTTTATACAGGCAATACTGGCTTCGCACAAAACCGCAAGACCATAAACATGACCATGTAAGCTGAAATTC
TGCAAAACAATCTTCATGATGAATGGGAAAAACTATTATTATATTGTTCCATGACCTTTAAAATTTTTTT
TGTTGTTAAAACCTTAAAAACTCTGTTATTATCAATAACAACGGCATTGGGAAATGAAAAATAGTAAAGC
TAGTATTTAGTATGTGGTAAATTAAATCATTAGAAACATCGAGAATGAAAGTGTGTTATCAAGAGTAGTT
TGAACAACACTTGTGTGTTCTTCTTGCATAACTTTGGATACAGAGCAAGCATCTTCTCTATGGCTTGGTG
AATTGTCATACCCCTTTCTAAGTTTGGATCGGTTTCTACCGTTTTATCCTTTGCACTCTCAATGTCATGA
AAGAATTCCTTTAATGTTTTTTTGAATGTTTAAAGTTTATTTTATTGCCAGTCACTTCCTCTGGGACATC
TTTATTCTTTTTGTTACATCCTCCTCACTTTCCTCATTTATATCAGTAAGTCCACCTTCGCTGAGTTCTT
CTGGCTTCATCTCTAGAGTTTCTTGCATTGCAGTAATGTCGACATTCGCACAATCAGCTATTTCTTCTCT
TACTCCATTTATGGTCTATTCAAATTTCACTCCTAGCATTTGTCAGTTTTTATTTCTTTACTCTTTCATC
TTTCTTGCCTAGTTTCCTCTTTTGATTTATCCATTATAAAATGTCACATGGGTTTATCACTGGGAAACAA
GGAGGTAACAAAACTCCATACTTTGCTGTCTGTGCATGACCTAAATATCAGATGTACAGTGACTAGTCAC
CAACAGTCTTCAAAAGAAGTGACATGGTTGATCACTGATCATGATGGGACATCTGCTATTTACATAGTTA
TTTTTGAACTGAAGAAATAGCAGTGAAGTTGTACTTTATGCAGTTACTCAGTTAATATATTGTGGTAATT
GAAATTTGGACTGTTTATGAGGGATTTATTTGATTAAACCATGGTAACTGGAATTCATATCAGAATAGTG
CAAAGTGAGGACTGCTGTACTTGATTCCAAATTTAAAACTGTATTCTAGGCATCTTTAATTTTTTTTTTT
CAACTTTCCCCTCCCTGCTCAAAATTACTCCACAGAGATTTTGGTTACGGCTCAGATGTCTTTGAGTAGA
TGTTCCTGTAGAAATCATTGTTAGAAAATTCAAGGAAAAAAGTTCTCATTACACTACCTTGAGATCCTAC
CAAGTCCTTGAGTTTTGACTTGAGGACAGCTTAATAATGAAAGTAATTCAGCCATGAAAGCATATTTAAA
ATAAGTAATGGAACAGGAAATTTCTTCATAGATTAGAAAAATTATTCTGAAAAAGTGAAGACATAGCCTA
TCTTGGACTAGGAAATTTCCATCCAAGAAAGTAAAATATACAATAAATATTATCACAAAGAATCATTCTG
TGACCTGGTTGATGGCCCCTGGTAATAAGACTTTGTTATTAAGTTTACTGTGAATTCTTTTTTTTTTTTT
TTTTTTTTTTTTTTTTTTTTGAGACGGAGTCTCGCTCTGTTGCCCAGGCTGGAGTGCAGTGGCGTGATCT
CGGCTCACTGCAACCTCCGCCTCCCAGGTTCAAGCGATTCTTCTGTCTCAGCCTCCCGAGTAGCTACAGG
CTACTGGCGTGCACCACCATGCCTGGGTAATTTTTGTATTTTTAGTAGTGATGGGGTTTCACCATATTGG
CCAGGCTGGTCTCGAACTCCTGACCTCGTGATCCGTCTGTCTCGGCCTCCCAAAGTGCTGAGATTACAGG
CATTAGCCACCATGCATGGCCCCTTACAGTGAATTCTGTTCAATAATCTTGAACTCCCAGTGCTTTCCCT
TGGTCCTGTCCATATAATGATCACATTCTGTTATTAAATAATGTGCTTATGTGCTGATTTTATTGTAGGA
GGAATTGAACAAATCTATAGCTCAGCATTTTACTCTTTTGTAGAAATCCCTTGACCCTTTCCAAATCCAG
ACATCACAACCTTCTCCATATACTCAAAAATTGTGAAATCCACAAGGCAGGGAAAGTTGATAATGAAAAA
AAGGAAGTTTGGTCTGTTGGACATCCTCTTCTTTCATCTTTTATGTTCAAAGTACTTGATGCACAAAGCC
TGGACTCTTTTATCTCTGTCCTATAGACTGATGTCTAGTCAGTATTCATTGTGACAAAATTGTTTCATTA
GAGCAAGTTGGATAGTGTATGGAGATTATTATAAAATGATTTAACTTGTCTTCTGTCAAGTAATTTTATA
ATTGGTATTAATGTTCATTTTTTTCCAGTACCAGTTTCAGAAGCTTTCATTTGTGTATGCATGTGTGTGT
TAAATAAGTGTATTAGATACTTGAAAAATAGTAATTTAAATTTAAACAATTTAAAAAATGAAATTGTAAT
TTTATGTGTAAAGGTGTCTGATGTGCTTTATTCTGCACTGAAAACAATATTCATTTACAGCTCAACAGAC
ACCAAATATATTCTAAAATTACTTTCCTAGAGTTATCAGAAAACAGCACTGTAATAATGAAATCAAACCC
ATCTTTCTTTATGATTTATTCTTAGTCTGATACGCATCAGCCTGGTAAGCCTTCCTGTCTCTCTGCTTAC
TTACCAATCACTCCAAATGTCATGTCTTTGGGCAGGCATTAAATTCTTGGGTTTTGGGTTTTGTTGGATG
GACTGCAGTCTGTGTGAGCCTATATGGGTGTGTCAAATCCAGTCTTTGGGGTGTCATGGAAACTTAGCAT
GATAGACTTGATTTTATCCCCAAGTTGACTTGGTAATTTCATTAGATTTCATCAGTCACAACCTGCATTT
TATCTTGTATGTGCTGTCTATTGGTCACAAAATCAGAAAACCTTCTTGTCCATTCATAACATTAGCTGTT
TTTTCAGGTGGCTAGAGGGACATGTCATTGCTTCATCTGCATGAATTTGAAAGATTAAATGCATAAAGGA
ATTTTCTTAGAGTAGAGTAGGCCTTCACCATCTCTTTAACTGGGAAAGAAGTTTTGGGAGTAACATACTC
ATCACTCACACCCCCCTCCCCTCAAACACACACACTCACCCATTAGAATGTAAGGGCCTTGAGGCAGGCC
TTGTGTTTGCTTTTCGTGTTCACTGTCAGCATTTAGAATAGTGATAGTCATATAGATGGATAGTGCTCAA
TCAATTATTGTCAAATAAAGTAATCTACTTGTTCCTTGATTTAGACTAGCAAAAGGGGCTGGTACATTGT
AGGTACACAGTAAATATTTGTAGAACAGATGAATGAACCAACCCAACAGATTTCTTAGAAGTAGCCTGCT
TTTGGTTACTAATTATTTTATAGAACATATAAAAGAAAATTTTAGAATACCTAATTTGTTCACAAAAATG
TTATATTGTCTCCCCTGATACTTGGTATTCCTGTGTCAGGCTGAGCACTAAGGTTATACATTTTTTGTTA
AGCATGTAATTACTGTTATTTCTCATGTACTTTATTTTGTGTATTGATTTCAGTAGTTGTAGCTGAGGGT
AATCCTTAAGAGCATGCAGATTTTAAAAATTACATAGATTGCCTTGCCAAGCCGTCTGAAAGTCATACTA
GAAAATTTGATAAGAGCCTGCTGTTAACAACACATTGAGTTATTTTTATCTTAATGCTAAGTGGAGAATT
ACTTGAATTTATTTTTTTTGCTGCATTTAATGTTTTGATACATTCAATAAATAAGGATAAATCCACTTAC
TGAAGGACAAGAAAAGATCAATAAGAGATCCTTGAGGTTATTGGGTTCTATGGCATCTATTATATCAAAT
TCCTTGCCCTTTCACTATACTGAGAAATTTGACGACTGGGCTAGCAACAAGATACCTACCCCACATTAGC
TGTGGTACCTGCTGAAATGTGACCATGTGAGCAAAGAAAATGAGCCAAAAGAAACTCTTTCTGAAGATTC
ACTGAAGAAAACCTGGAGATACTCTTTCATTAGTAGACCCCACAGAGACAGGTGGAGTGTCCGTTATCCA
GAATGCCTGGGACCAGAAGTGTTTTGGATTTCAGATTTTTTCACACTTGGGATCCTCAACATGTGTATTT
GTTGGAAATGGCTACTTAATTTAAGGAAAAGTTTAAGGTGGCCTGGAAAATAAGTAGTAGTTGAACTAAT
CAACAGGAACCAAAACTACTTTCAATATATAGTGTACTTATACTCAAAGAGAGACGGACCATTTTATGGT
GGAACCCTGTCTATGGTAGTATTTTGTGATGTTTTATTTTTGTTGCTATTGTTTGCACTGTTTTCTCCTA
TAACAGCTCTTCTAAGCCTTAAGAGGATAAATTTTATATGAGATTCAAACTCTTATTTTTGTTAAGAAGG
ATGTAAGTCACCAGGCATGGTGGCTCACACCTGTAATCCCAGCACTTTGGGAGGCCGTGGCAGGTGGATC
ATGAGGTAAGGAGCTCGAGATCAGCCTGGCCAACAATGTGAAACCCCGTCTCTACTAAAAAAAAAAAAAA
AAGAATTCAAAAATTAGCCAGGCCTGGTGGCGCATGCCTGTAGTCCCAGCTACTCGGGAGGCTGAGGCAG
GAGAATCATTGAACCTGGGAGGCAGAGTTTGCAGTGAGCCGAGATTGTGCCACTGCACTCCAGCCTGGGT
AACGGAGTGAGAGACTCTGTCTCCAAAAAAAAAAAAAAAGAAAAGAAAAAGAAAAATATAGAAGAAGAAC
ATAAGTCAACCTTTTTCCATGAAATTATACTGTACTCTAAGGCAAATTCTTGTTGCTTGATCTTAGTAGC
TTTTTTATGCATTGACTTAAACCTGGAAGAGTTTCTTATGAATGTTTGATTGACTGTGAGGTGCATTTGA
AACAGCTTCTACTTTATAACCCTTTGCAGAACTTTCAAGCTCTATTTAGATAACAATATATATGGGATTA
AAATGGAAAATGTGACATATGTCTAGAGAAAGTTCCTTTTTCTGTGTATGTGCCTTTTGTGACTTATACA
CTCAGTTTCATTCTAGCCAGCATTTGAGCCTAGAAAATAGGGAAGTTATGAAAACTCTGTGCCTTATAAG
AAGGCAAGGCTATGACTGAGTATTAGACACATAAGTCCAGGGCTGGGCGGAAGTAATGAATCAAATAAAA
ACTTGGGGAACTTGCCATAGGATGTTTTCTGATTAATATGGGTTTCTCTTCTCTGATTAATGTGGATTTC
AGTTACTCAACTGTGTAACTGAAATCCACATTAATAAATTGGTTATTTATTGCTTAGGACTTATCTCCCC
ATAAAGAGACTAAAATTGAGGTTATAAATTATGGGTAGATAGAAATTCCAGAAATATTTAGGTACCTGTC
ATATACCTTAAACATAGATTTTTATCACAATCATTTAGGGGCTTATTTATTGCTTTTACTTTATTTAATG
TTTGTCACTGTAGAAGAAAAAAAACTAAATGCTAAATATAACATTTAAAATATTTTCCCCTTCATGACAG
CCACCAATTGATTACTGTCATGGAGACTATCTCTATGTGGATGACACACAGCAGCATTTCAATCACACGC
TGTTTCTTTCCGCTACTCAGTTTGCTTTTAGGTTGCCTTAAACAACTTCCTTGGTGAAAAAATCAACTTC
AATATTAACCAAAATTTAAAAGATTCATATATAGTAAAAGAACTAATATTCGCAGATTGACCCATTCCAT
TCTTTCAGAGAAGGTATGAGATACTTGACAGTGGAGCTAGCAGCAGAAACAATAACATGTATGATGAATC
CCATTGAGTCTTTGCAGTTTATTTTTATTAAAATATTTTAATTGAACAATTTAAGCTTTTTTTCTTCATC
AGGATTTCACAAGGGTGTAATCTGCCCAGTTTTATTCTGTCATTTTTTTCAAAACAAAATAACTAGATTT
CCAGATTGTTTACTAATTTTTTATAAGGTAGGACAAAATTCTCTTTTTCTCAATATTGTTAATTAACCCA
TTATTTCCCATTAGTATTATGTACATCAGTGTTGTGTTGTCACTTGGAGTGGTTTTGCTCATCGGGGGCA
TTGACAATGTCTGAAAACAGTTTGATTAACATAACTGGCATCTAGTTACTAGCATCTAGTATGTAGAGGC
CAGGGATGCTCCTCAACATTCTATATTATGCAGAGCAGTGTCCCCCCTCCCCCCAAAAACTTATCTAGCT
CAATTTATTAGTAATACATCAACCGAGAAAGACTGATGTATAGAACCTCATTTGTTAGTGTAGGAAAATA
AGGTGTCACCTATAAATTCACCATCCATATTTATACTGCCGTGACGTTATCCATTTGCTTATGAAAGAGA
TGTGAGGTGACTTGATGATATTAAGGAGTTGTTCCTCATAAGTTATTACATTATAGTACTTCTGTCAGTT
TGTCTCTGTACCTTACAAGTTATTAAAATGGCTTCACTTGTGATTGAGTTCATATAATTCTTTGTTTTTC
TTTTTTAAGCACTTAAATATAGATCCGGTGGTATGGATGAGAAAACAATTGCTTTACTTGTTGCTGGACT
AGTGATCACTGTCATTGTCATTGTTGGAGCCATTCTTTTCGTCCCAGGTAAGATGTGCAGTTCCTAGGCA
GGAACGCAGGAGGTAGATGAGTGCATTCCAAGGTGAGGAGGGCTGCATTAGTTTCCTAGGGCTGTTGGAA
CAGATGACCACAAACTGGGTGGCTTAAACAATAGCAATTCCCTTTCCATGCTGGAGGCCATAAGTCTGAA
ATCAGGACGTCATCAGGGCCACACTCCCTCTCAAGGCTGTATGGAAGAATCCATTTCTTGGCTCTTCTAG
CTTCTGGAATTTGCTGAAGAATGACAGTGTTTATCATTCCTTAGTTTGCGGCAGCATAACTCCACCCTCT
GCCTCTCTGGTCACATTGCCTCCTCCTTCCTCTATGTCTTTGCCTCTGTGTTTTTGTTAAATCTCCCTCA
GCCTCTCTCTTATGAGCACATTTGTCATTGGAATTAGAGCCCACTCATATAATCCAGGGTAAGCTCCTCC
TTTCAGATCCTTAACTTCATCATATCTTTTGCCATATAATGCAGTATTCACTCTTTTGCTGTATTAGGTA
ATATTCACAGGTTTTAGGGATTAGGAGGTAGAAATAATTTTAGGGCCATCATTCAACTCACTACAGAGGC
AAACTTCATTGGCCAAGACATGAAAGTAGAATGAATGTGGGAACCAAGTCTGGAAATTGGCAGTTGCATT
TGGAGGATGGGAATGTGAGTGGGAAATGAGGATATGTAGCACATGTAAAGAAAAGCAAAGGAAGGCTGGG
ACTCAACCTAATAACTATAGCACACCATCGTTTGTGGAGAAGAATGCAGTGCAGGTGTAATATTTAGGAA
CATGGGCTTGGCAGCTCTATTGGTATCTGAAGTTAGTGAAGACAGCAGGAAGAAGGAGGTGAATTCAGAA
GACAACTTGAAGGAACAATTGATTTGACTTTGGGGCATACTGAATACAGCAGATGAAGGGAACAGTAAAA
GATGAATACACAGTTTCTCACTGGAATTCTGTAAACTAGTTAAGAAGGAGGCAGAGCCAATTCAAGGTTT
CAGCATGTTGAAACTTAAATGTTGAGATTTAGATGTCTTAAGTTTGGTCTTAATCAAAAAATAGTCACTA
ATTTTGTGTGAGGTTTTCAGGGAAACGTATATTTTTAAAATTTTCACAGTTGTCAAGCACAGAATTTAGA
TTAGTACATTTAAAAAGTATGTAGGAAAATATTTTAAATGTTTTTATTTATTGACAGGTGAATATTCATT
AAAGAATGCTACTGGCCTTGGTTTAATTGTGACTTCTACAGGGATATTAATATTACTTCACTACTATGTG
TTTAGTACAGGTGAGTTTTCATTGTTACAGTATGATTTTGTCTACCTTTTTCATTTACAAAACAGCAGTT
TTGGTGAAAATGCTCATATAAATTTTTTACAACTCAATAAGAGTAGGTTTATTAAAAGATTTTTCATGCT
ATTCTTAGTGACATTTTCCCATTCATATTAATTTTAAGGTTATTCTAGGTTAGCTGTTTTGTAAAAAGTG
ACTTTCATATGTATTTGATGCCAATCAGCATAATTTTAAATTATGCCATATAACTTCTTAATGATTATTT
TCATATTCTAATTTCAGTTTTTTGAAGTTATAAGTGGATGTTAAACGCAGTCTTTCTCTTCTTTTGCTAA
GCTTCTGCTTCTGCATTAAGAAGACTGGTTCTATAAAATGGAATTATGAATCACAAATAGTAGAAACATA
TTTGTTTTAATTATTAGCAAACCTTAATACTGTAGTTTTAAGAGATGGTATGGAAATCCAAACTATAATC
AGTATCATTTTCACTGCATTTTGAAAGTAGATCACTACCATATTTAGTTATTACTATTAAAGAGTCACAT
TTAACACTTGGTCTTAGAGCCTATCTCTGGCTCGTTGTACATTGGAAAGGTATTAGTTTGTTATAAAATG
ACATCTAGAGATAGAGAGCATGATGTTTACAGTCAGGTATTTGCATAATGGTTCCCTCAGCAAGAGCAAT
TCCATGTGCATGTGGAGAAGACATTCATAATAGCCATTCATATTGTAAAATGCACAAGTGTGGTAAAAGC
AGCATTGTTCTAAGATTTAGGGTAAAAACTTCCAGTCCAGCTTAGCTGACTGTGAAAATTAAAGGCTGCC
GAATGTGTGAATTTGGAGCTGACTACTTGTGTGGAAGGGTTAGATCACTGAGTTAACATCTACCGTCAAA
CAATGAACTCCAGAGTCAGTCTTTGGTCTTAGGAGATCCCTGATTATACCAAATGCAAGTGGAAGTTATT
GCTTTTTAAATACTCTTGACATGCTTCTGTTACATCCTTTTCCTTCCTCCAGCGATTGGATTAACCTCCT
TCGTCATTGCCATATTGGTTATTCAGGTGATAGCCTATATCCTCGCTGTGGTTGGACTGAGTCTCTGTAT
TGCGGGTAAGAGTCATCTTTCTGTAGACCTAATTTGGGTTACTTTTGGACAGAGCTCTTCTTCCTTTTTC
TTTTTCTTTCTCTCTTTTTAAAAATATATAGACTTGATTTTTTTTTTTAGTGCAGTTTTTGGTTCATGGC
AAAATTGAGTGGAAAGTACATTCACATGGATATTTTTATGTGGACATAAGTTTTCAATTCCATTTGGGCA
AATACCAAGGAGTGCAATTACGGGACTGTAAAGAGTATGTTGAAATAAACTGCCAAACTCTCTGTATCAA
AATAGCTGTACCATTTTGTATTATCACCACCAATAAATAAGAGTTTTTGTTCATTCATATACTTGACAGC
ATTTAGCATTCTGAGCTTTTCTTTTTAAACTTCCAATTTACCCCAGTGGTAATAGTGCTTTCCTCCTCGC
TACAAAGATATTAGCTGTATATATGGCTTGGTGGCTGATTGTTCTAGCACCCAAACTGATATGCCTGTAT
TTGTGGAAGAGCTTTTAAAATAACTGGGCTCAAATTGGTTGGAGCCTTAGACTTGAAACACCAGTTCCCA
TTTCTTGATATGATAAGGTATGTGTTATGCAAAGGAGGGCTTTGTTCTTCTAATAATTTTGAGTCATTTT
ACTGGTTAAGTTAATAAACATATATGGATAATTTTTGTTTTTTGATCGTTAGAATAACTCTCTTAAAACT
TGGGATTATTACTGTTTTTTAGTAAGTTATTTCATATGCTTTTCTAATACAGAATTTTATTTGTTTTTAC
AGCGTGTATACCAATGCATGGCCCTCTTCTGATTTCAGGTTTGAGTATCTTAGCTCTAGCACAATTACTT
GGACTAGTTTATATGAAATTTGTGGGTAAGTCAATCTTATTTTCATTAACCTATGCCAATAATTTCAGAT
ATATCACTTAGAAAATGCTTTTTAGTTTGTTCTTCCAGTTTAGGACCAAAAATGAGAAAATACAATTGGA
GTGATTCGAGGATAATTAAAGAGGGTAGAAGACATATAGGATTTTTAGTTGGTTCTTCCAGTTTAAGACC
AGAAGTCAGAAAATACAATTGGAATGATTTGAGGGTAATTAAAGAGGGTAGAAGACATATAGGATTAATG
AAAATTTGGTTTCCAAAGTAGTTTAAAGGAAAATGGCTTTATCTATTAGAATGTGTACCTTTTTATACTA
AGTAAAAGGGGAGAGATCTTTGAGGATCCATTTTAAGTAATAGAATAGGATTTTTAATTGTTCCAGTGTT
TCTGTGATAGAGCTGTCCTGCACAGACCTGTTTGTTTGTCACTTGCTCTTTTTCTTGCAGACATAGACAC
CCCAGACAGGAATTAAAATTCACAATCTATCAATTTTGTTCATTTAAAGAGCAGTGACCTCTAATGCATG
ACTTTAAAACAGTTCTAGTTAAAACCAATATAATGAAAACATTGAGTTTCAAAATTTAGGCTTTTACTCC
TTTTAAAATCAATTATTAGTAAGTATGGAATTTACTTCATTGTTTCTAACTTGTATATTTAATCTGCCAA
TTTTCAAGTAACATTTCTGCATAAATTCTTATTTTTTATTGAGATATATGTACACAGAGAGATATTTTCA
ATTGTGCCTGAAACTAATGTTATCTTACCTAAGCTCAAGATGTTCCCAATAATGTAATTTATATTAGTTT
CCGTTTTTTAAAAAAATTATATTTTTATGAAATAAAACATACTCTTAACCACCTATCAAAATAATCAAAA
GTTATAAATTAATGGAGTAAAAAAATAGTGTTTCTGCTTTGCTTTAGGTAAACTTTGCTGTATGTGTTTC
TAAAACTTAATACGAAACTTGAATTGTTATAGTCAAATAATTTCTCATATGACTCACATAATAGTTTCAA
AAAACTTTTACCTTTATTTCTGAACTTTGGTTCTTGATGATTGTTAATTGAATTCAATTCTGTCATATAT
TCTGTGTCTTTCTTTAATTAATGCTTATTAGATAAATAATTAAAATACTTAACTAAAATCTGCGTATCCT
TAGCATATGAGTTCATAAGTCTTAGTTGTTGCTCAATGAAATTTTCTAATTTTATACCACATAATGCCAT
AAAATACAATGGAGACATCTAAAGCAGAATGGAATTCATGTGGTAGCTACAGTGAACATCTTGAATGTTG
GTGCATATTCTATTTTTGTTACATCTTCCAATCACCATGTGTCTGGTTCTGGAAGATGACACTCCTGGTT
TTGTTGCTCCCCACAAATGCCTGAGAATAGTGTGTGATTTGCAGTATCCATACAACTCTGGTGAAGTAGT
ATGAGATACCTTTGGCTGACGGGCAGCACGCTCTTATTTTTTCCTCACTATCTGGGTTGTCCTCCCTTTT
ACTCCCATGCCACCCCATGCCTTCCATATCTAGCATAGAATGATCTTCAGTACAGTTGCCAGCAGGTCTG
GTGACAATGTCTCAAGTGGAACTAAGCATTGTCTATCTGCCACCTCCTTAACTTCACTCTCCTGCCTTCT
CCATCACTTACGTTCCTCCAAGCCTGTGAAACCACTGTACTGTACCTGTCACTGTTCTGACATTAAAATT
AAAATGACTTAAATCTTGACAAGTACCCCAAATTATTTTTTCTTTGTCATAGGTTAGCATATAAGTATAC
TATATGCTAAAATTTATGCTATATGTTTAAAATTTAGTGCAATTTTATTGATAGTGTCCTAATTTTATTG
ATAGTATCCTAAATTAACTTTTTAAATCAACTTGTCTGATGCCAGGGTTCAGAGGGACACCTACAGTCAG
TTGAAAGGCAAGAAGAGACAAGGTACAGGAAAGTTGCTCTTTAGATAACATGGTAGACTAGGAGGAACAT
TAATATGGTTTGCTTATATAATCTGACTGTGTAAATCTGAATCTATGTAACATTAAGGTTGCAAATTTGA
GCGTTTATATTAGGAAGTTAAAATTTTAAGTGTCTCCAAAATAATTTTTACTCATTGCACGTGTTCTGTT
TTAGAAAAGCCTAATGATTGTGTTTTGATTTAAATGCAATAAAATCCTCAAATAGTTAAAAATCCAAGCT
TTCTCTTCAAGAAGAAGTTAATGTCGCTATGAGATTTTTAACTTTTATAATTTTTATTATTTCCAACTTT
AAATTTGTAGCCTTAATTTGCTATTTTAAAGAGTAGGCCTTTCACTTTCTACAACTTTCTGTGAAAGTGA
CTTTCACTTTCTATTTTTTAACTTTTTAAACTGTGTTGTATTTTTTTTCTTTTATTGGAAGCATTTTAAT
TTTATAAGATGAGAAAAAGGACTGGGCACAATAACTTAATGTGAAAGCATAGAAAAGATTACAAGAACCT
AACCAAACTCACTAAAGTTGGGCTTGTTGTTTGTAGAGAACGTTTATATAATTATAAGGATCAATACTTT
CTCATTTTTAAAGCCATTACCAGTTAGTTCAATATAAGGGCATATAGTGTTTTGATACAAATCAATCTGG
TAGCAGTAAGTACCATATTTACCACAACATCCCAGATATTTTAGAATGATGCAGATGCAGAATATATACG
TAGAATTTATATCTATGTATAGATACAAATTCAGATATTTTCTTGTTCAATTTAAGGAGAGGTAAATTTG
GTATCAATAGAAAAAATGTTTCTGAAAAATTTAAACCCTGGAAATGTATTTATGGCATGGAGTCAGATGT
TTCAGGGAGAGAAGAACAAATCAAGAAGCATTGCAAGTATGCTCATATGGAATGCTTAAGGCTTGTGGTT
AAAAAATATATATATATGGCTGTCAATGTCTTAGGCTCATGGTAGCAGCAGAAATCGTAATAATTCTTTT
GTCACATGGGTTATATCCATATTGGAGAGAATTAACTCAGGTGAAATTAACTTGTACACTGTTTGGTTTT
ATAATATTTAGAGGGATCACAACTGACTGATGTCCCTTTGAAGTACCATTCTTCATAAATCTTTTTTTTT
CAGAATGGGCCAGCCAACTGTGACATCCCTTGGATCGGAGATTTAGAACTAGAAAGTATTCTTTCTACAT
TATTAGGGAAGAAAAGGAGTTACTTGGCGGTTAGCAATATTCTATTTTGTTTTGTTTTGTTTTTAGAGAC
AGGGTCTCATTATGTTGACCAGGCTGGCCTCGAGCTCCTGGGCTCAAGCAATGCTCCCACCTCAGCCTCC
CAAGTAGCTGGGACTACAGGCATGTGCCACTACACCTGGCAGTGTTTATTCTGATAAATACATTTATGAG
CTCAAAAATGTAACTCTAAAACCTTATCTCTGAACTTCCATATTACCATCAGAAATTTAGATAGTTGTTT
AGTTCTCTTTTTCTTTGTAGAACATAGATATAAGGCATGGTTTCATTGAAGTCAGTTGTATATACATGTA
ACTATCCTGATGTTCCCAAATAAAGCTCTGTATTTCTGCTTAGTTTATTGGGGAGGCTGCTAAATGTAGT
GCATCCCAACCCATTTTACCCTGTTCTACTTTAAAAAGAGGTTGGCTTCTTGTTTGGATACAAGGACCAA
GTCACTCCCCCAGGTTCCTCCACAGTAAGGGAGGCCTATTTAAAGCCGCCCATGGCACTAACAGAAACTG
GACTCCTATGAGCTCAGATACATAACTGGGCCTCACAGGGGTGGGACAGTATGTAGTCTAGGAATTGGAA
GGATCCATTCCATATCAAAGAACTGAAGCATCGTGTTGCCCTCTCAGCAGCAAGAGTAAGGTGATGCCCC
TGTCAGTTATAGTTCCTGAGTTCCTCTGTCTTTGATTCTTTGCCTATTAGCCAGCTAGCTCACCCTCTTG
TTTATGCCACTGTTTTTTATCCTATTCATGCCTTCTCACAGACAACTTTTCTTACCTACAGCTTTGGACT
CATCCTTGTCTCCTTTCTGTTTCTTTTTCACTTTCCCTTCCCATCACCAACTTTCTGGGTTTTTTTCTGT
TTCTTCTTAGAGTCCAGTGGCAGGGAGAAACTTGTCAGTCCAGTCTGTTGCCATTTTTCCTGTTTGAGAA
AGACTCACCAGCTTTTGGCTGGCTCACAGATTGGCTTTCCTTGGGTCAGGACCCACCCTTTTCCCTGCCA
GCTTTGGAAGCTTGACAGAATTCGAGTGTGCAGTGGTGGTAAATAAATAGTAAGGAACACAGAGCAGTCC
TGGAGGCGTGCCTCCATCTGCTGATGAGAAAATCCAGTGCTGTCATCCAGCCCAGGTCCCAGCGGAATGG
GCCTCTCTGTTCAGTAGGATCCCCCTCCTGCTGAGTGGTTCATGGCATGTTTCTGTTCAACGCTTTTCCA
TCTGTAGGATTCTTATTCTGTATTTATTTGTTTTTTTGGGTTTTTTTATTTTTTGAGATGGAGTCTCGCT
CTGTCGCCCAGGCTGGAGTGCAGTGGCACGACCCCAGCTCGCTGCAGCCTCTGCCTCCCAGGACGAGGGA
GATCCTCCCACCTCAGCCTTCCACGTAGCTGGGACTACAGGCATGCACCACAGGCATGCACCACCACGCC
AGCTAATTTTTGTATTTTTGGTAGAGACAGGGTTGCATCATGTTGCCCAGGCTGGTCTTGAATGCCTGAG
CTCAAGCAATCTATTTGCCTTGGCCTCCCAAAGTGCTGGGATTACAGGCATGAGCCACCACGGCCAGCCT
TCTCATTTGTTTTTTTTATAAGGAAGCTATCTCTTCTTCCCTCCCCAACTAGGGTATTCTTTTTCCCTTT
CGTCACTTTGCTCATGTACTGTATTCCTTCAACTTCATTAATGAATCCATTTGGAAGCAGTGAAAAAGGC
AACTCAGAAAGCTAAGAAGAAATAGATAGAGGAATACTCAGAGCTATCTGAGTATTTTCTTTAGTTTGTT
AGCTCTTTGGAGCTTTGAAACTGGAAAGACCCAGGGAGTGATGTGGAGAAAGAGACTGAGCTTGTAAGAC
ACAGGAGCAGTGAGCTAAGGGAGATGGAGTAGTGGGGACAAATTCTGGCACATTCTGTCTACACTCTGGG
TAGATAGAGGAGGGAGGATGGAGCACCCATGGTGGGGGTATGTTGGTGACAGCATTTTCCCACCAGCCAG
TGTAACAAGTGGCTGATTTGGGGGAAAGATGGCATAAACAAATGAGAGAATGTGTTTACTATTTGATGTA
GATGGGTTATTTGCTTCATTTTTCAAATCAGTGTATATAATCAAGAATATTCAGCATGTTTGAATAGACT
GTCAGAGCTGGAACTCTTTCATTAACATCTCTGGCACCTTTAGTTTTAGCCCTGAACATTTTATCTTAAA
ATTAAACATTACCAAATGCCTTAGTTTATTTCATTTATTAAATTTATATTCTTATTTGTTATTTATATCA
GCTTCCAATCAGAAGACTATACAACCTCCTAGGGTAAGTTAAAGTTTATTAAATGAATTGTGAATGATCA
TTTGAGGGATTAGACTGAGGAACTTGGTAATTGAGATATTTTGCTATCTGTTTTGTCTCACGTCAAATTA
AGAGAATGTTGAAGTCATTGCATGACCTTTGCATGAATGGGTCCAGTTCTATTTTAAAACCTGTGTTTGG
TCATTTTAGTGTCAATGGGATGGAATAAATGATTTCTTAAGATTGTACTGACTTCTCACACCCAAAACTG
GAAAGTAGGAATAATGGCTATATTATCTCTGCAATCAGAAGGAAGCTGATTCCAATATATCACCTCACCT
GTTGGATTCATTGGATGTGCATACACAGAATGACAATTTCAGGCTTAAAAATGAGGAGAAATCTATACTA
AGTTGACATCACTGATAATTATAATCTATAAAATAAATGTAAATATTGCTGAAAACATCTGTTCGGAGTT
ATGATTCGATCCTCTCCCATACAAATGTTTTATAAACATTTTTTCCCTTAAAACTGTGCTTAAGGTTTCA
TTGTACCTTAGATACCTTATTAAGCCATCTGAGGAAATTGCAAGAAAGGAGTAATTTTAGGAGGGCATAA
ATGAAGAGAAAAGCATATTTATAAACATAGACTTATCACAGTGACAGGCCCAAGAGGTATGTTGTGGACA
TAAGCTCTGGAAAGGATTATATTGTACTTAGCTTCCTATAACGGAGTGATGATAGATGTATCTGGAATGC
CAAAGAGAGTCTTCTGTCTGTGGCCGGAGGTAGAGGTCACATATGCTTCTAAGTCTGACAAGCCTCATTG
TGCCTAAAGGGCAGGTGGGGCGGGTATGTGTGTCTACCACAGGGGTATATTCTAGAAAGATTGGTGCCAT
AGCTATGTTGGTCACAAGAGGCCAGCAACTTAACTGGACCTGTGAATCCTAACACATTTTCTTTCCCAGT
TACTGAGTTCAATTTGCGATACTTAAAGATGATTCTGTTTTGCTTCCACCTCTTCACTGTGTTATTTATT
CTGTTGTTGCTATTTATGCTTGCACTTTCATATTTTTAGAAGTTAGAATTTCTTGAGCTGAGAGTGGTGA
AGTGGGAAATTCTGTTTAGAAAAATAATATTAAGAGAATAATACAGTTATTATTAAACTATTAACCCGAC
TTGGCAAGCTTTGCTTTAACATTTACAGGCTTATTTCGTTGTTTTGTTTTTTCTTTTTTTTTTGACTGTA
GGATTTACTGCTTACTACGTAATTTAAATATTAGCATATATAAGTTTTACTATAAAATGACATGACATAA
ATTATATTTTTATGTAAATATATTTTAAATATTTTTCAGAAAGCTGTAGAGGAACCCCTTAATGGTATGT
GGTTATTTCACTCTTAATCCTTTACCAGATCATAATTTGATCTGGCCCGCAAAACAGTTAGAATGCCCTG
TCTATGCCTTAGGAAGAATCTAGGTTTTTTTCCTCTTTTTTTCTTTCCTTGGCATCTCTACTCTTGATTA
TTCATCAAGAATTATGGGCTGGGTGCGGTGGCTCACGCTTGCGATCCCGGCACCTTGGGAGGCCAAGGCG
GGCAGATCACGAGGTCAAGAGATTGAGACCATCCTGGCCAACATGTTGAAACCCTGTCTCTACTGAAAAT
ACAAAAATTACCTGGGCATGGTGGTGTGTATCTGCAGTCCCAGCTACTCGGGAGGCTGAGGCAGGAGAAT
TGCTTGAACCCGGAAGGCAGATGTTGCAGTGAGTTGAGATCATGCCACCGCACTCCAGCCTGGTGACAGA
GTGAGACTCTGTCTCAAAAAAAATAAAAAAGAATTATGAATATTACTTTTATAATATTCTCACCACTGGG
AAAAATGCACTATTCTGTGTCTAAGTAGCTGCTTACCTTAACCAAGTGATATTTGGGCAAGGGGATCGTT
GCCTTTTGCTACTGGTTGAGACGAAGCATGGCACCCCCTAGTAGAGAAGGATCCCAATTACTTCCAATTT
GTGATGTACACATTTTAGAAAGATACAGGCTATTGCCACAGAGATAGACCAAAACATCTCATTTTCTTTC
TTTGTAAACCTAGATCTGATTTCCCAACTAAGTCTGTTTCCTTTGTGAATGCTGTGGGTATGATCCACAG
AAAGGCTACATAATGAAATGATAGCTTTACAATTAATTTGGCTGTAGAGTTGTAGACTAGTTAGCATATC
ATTGCATATTTGTTTATTTAGAAATGATTTCCAATTGTGGAACCTCACTAACTGCCTGCTTGGCTTGTTA
CTAATCCTAGCATTCAAAGAATCAAAAGGAATGATGAATGATGGTAAGTATAAAATGCACTTAATAATTA
TAGATCAGTTAAAACATGGACATTGGAAAACAAAAAAGCTTCTTGAAAATGTGGCTCTTTTTTAGTAAAA
GGGACACTGTCAGATGATAAAGGTTCACATTTCTTGATGTATACAACTTAAATCTACTTTGCTAAAAATT
GCAAAACTACTACTGTAAAAACTGTAGGGTGTCACGAATCAGACTCCAGTCATATGGCTCCCAGCAAAGA
GAAATTACCACTTTTTGTAAAATGTTTTTCAGATTCCGTGTCTGGTGACTGTAACTTTAAGATGCCTTTT
ATAAGGCACATAAATAATCTGGCACAAATCTTTATCATTTTGACAGAGTTTCTTTTATGCTTGTGTTGGT
GATTTTGTTGCATTTAACCCATGGGGACTTAACATCTCTGCTTTTCCAATCAGGAGCTTGGTTCTAACCT
TTTGGGAGATGATTAAAGAGAGAGGTTGAGAGAGAGAGAGAGAGAGAGAGAGAGAGAGAGAGAGAGAGAG
AGAGAGATGTTTCGTCCTCAGCTTTTGCTTCCATTTTTTTTTTAAGAACTCTGGGCTAATAACTTCTAAT
CTTTATAGAATATTTCAAAGAAATATATTTGTTCTTAAAGATACATAGGTTTGAGATATTGAGTGCTACA
AGCATTTATTTTGGTTTTACCTTAACATATTATGATTCCTCAGTTTTGTTGGCATTTAGTAATTATGTTT
ATGTTTTTATCTTATCAAAAAATGTCTTCTTACCTTTGATATTTATAATCACTCCTCGGTCATGTAAATA
GTTTGCTTTATATTTTACTGTTTTAAAGTCTGTGACCTTACCTGCCCTCTTCTGTAGCAAAGTGCAGCAT
TTAACTCAGGAAGCTATATTCCCCCCAAGTGTCATTAATATTTGCATAAGATTAAAAACACTCCAGTCGG
CCGGGCGCGGTGGCTCACGCCTGTAATCCCAGCACTTTGGGAGGCCGAGGCGGGCGGATCACAAGGTCAG
GAGATCGAGACCATCCTGGCTAACACGGTGAAACCCCGTCTCTACTAAAACTACAAAAAATTAGCCGGGC
ATGGTGGCGGGCACCTGTGGTCCCAGCTACTTGGGAGGCTGAGGCAGGAGAATGGCGTGAACCCGGGAGG
CAGAGCTTGCAGTGAGCCAAGATCGTGCCACTACACTCCAGCCTGGGTGACAGAGCAAGACTCCGTCTCA
AGAAAAAAAAAAAAAAAAAAAAAAAAAAAACACTCCAGTCATGCATTGGTGAACAAAGTTTAAAACAACG
TGTATTCAGCATGGAGTCACAGAATGATCCTACTTTTGTATGTTTGTGTCACAGCCTTTAAAAGCATGTC
TTGTTATATAAGCCATTACCCTCCTAAAAAAGACTATAGTTCACAGGAATAAGTTAAAAGACATAACAAA
ACATAAAATGACTAGTACCAGGAATTGTGACCATGGTTTGTTCTGGTAACTGTGGCATGGCATGGTTTGT
TCCAGGAATTGTGGCATGGTTTGTTCATGTTTACATTCTGATGTCCTATTTTTTTTTTTTAATTTCTATG
TCCTTTCCTTTTCCTTGGTGGTGTCATTGTTCTGTAGCTGTATGAAGAAACTAAACTTTTCTCCATTTTC
AGGAAAGCAATCTAAGAATCTTGAGTGCCTCTTCCTTTGTTAATTTCTCTTAAGATGTGACTTTTTTAAA
CTACTGCATCAGGAAATATTGTAAAACAGTTTTGCCTTGAATATTTGTGATGAAATCTACGATGATCTTC
AAGATTCTCTTAATTTTGCTAATATTCAGCTGATCAGAATTTGTTTTTAAAATGTCTGGCTGGTGGGTAC
TTCCCACTGACAACTGCTTATTGCTTACAGTATGTCTGCCTTGTCAATGAATGAGGTTCAGGGTGCTTCC
TAGGGATCAGAGTCAGTACCATTTTTCTCTTTCATCTACAGCTGATCAGATGTTTATTTTACTTACATTA
AATGAATGATGGAGATCCAAAGTGAATATTATAGAATATTATTCTAGGATCAACATCTTTTGCTTTGAAA
AATCAACATCTCTTGGCTTTTCCTCAGCCAACCCAGCAAACAGAGATTATCAGACTCTGTTGATTTTTTA
CTTTCATTTGGCATTGGCCTTTTTCTTACTGAAATTAAAAAGGCTAATGATTTGCCTGGTTTCTGTCTCT
GACCTTTGCAGGTCTATTTTCTTAATTTTTAGATACTATATATCTGAAACTTTTTTTAATGTGTCAACTT
TTTAATGGATAGAAAATAGACACGAATAGTGATTATGTGTTCATTTTTCAATTTTCCAGAATAACTGAAG
TGAAGTGATGGACTCCGATTTGGAGAGTAGTAAGACGTGAAAGGAATACACTTGTGTTTAAGCACCATGG
CCTTGATGATTCACTGTTGGGGAGAAGAAACAAGAAAAGTAACTGGTTGTCACCTATGAGACCCTTACGT
GATTGTTAGTTAAGTTTTTATTCAAAGCAGCTGTAATTTAGTTAATAAAATAATTATGATCTATGTTGTT
TGCCCAATTGAGATCCAGTTTTTTGTTGTTATTTTTAATCAATTAGGGGCAATAGTAGAATGGACAATTT
CCAAGAATGATGCCTTTCAGGTCCTAGGGCCTCTGGCCTCTAGGTAACCAGTTTAAATTGGTTCAGGGTG
ATAACTACTTAGCACTGCCCTGGTGATTACCCAGAGATATCTATGAAAACCAGTGGCTTCCATCAAACCT
TTGCCAACTCAGGTTCACAGCAGCTTTGGGCAGTTATGGCAGTATGGCATTAGCTGAGAGGTGTCTGCCA
CTTCTGGGTCAATGGAATAATAAATTAAGTACAGGCAGGAATTTGGTTGGGAGCATCTTGTATGATCTCC
GTATGATGTGATATTGATGGAGATAGTGGTCCTCATTCTTGGGGGTTGCCATTCCCACATTCCCCCTTCA
ACAAACAGTGTAACAGGTCCTTCCCAGATTTAGGGTACTTTTATTGATGGATATGTTTTCCTTTTATTCA
CATAACCCCTTGAAACCCTGTCTTGTCCTCCTGTTACTTGCTTCTGCTGTACAAGATGTAGCACCTTTTC
TCCTCTTTGAACATGGTCTAGTGACACGGTAGCACCAGTTGCAGGAAGGAGCCAGACTTGTTCTCAGAGC
ACTGTGTTCACACTTTTCAGCAAAAATAGCTATGGTTGTAACATATGTATTCCCTTCCTCTGATTTGAAG
GCAAAAATCTACAGTGTTTCTTCACTTCTTTTCTGATCTGGGGCATGAAAAAAGCAAGATTGAAATTTGA
ACTATGAGTCTCCTGCATGGCAACAAAATGTGTGTCACCATCAGGCCAACAGGCCAGCCCTTGAATGGGG
ATTTATTACTGTTGTATCTATGTTGCATGATAAACATTCATCACCTTCCTCCTGTAGTCCTGCCTCGTAC
TCCCCTTCCCCTATGATTGAAAAGTAAACAAAACCCACATTTCCTATCCTGGTTAGAAGAAAATTAATGT
TCTGACAGTTGTGATCGCCTGGAGTACTTTTAGACTTTTAGCATTCGTTTTTTACCTGTTTGTGGATGTG
TGTTTGTATGTGCATACGTATGAGATAGGCACATGCATCTTCTGTATGGACAAAGGTGGGGTACCTACAG
GAGAGCAAAGGTTAATTTTGTGCTTTTAGTAAAAACATTTAAATACAAAGTTCTTTATTGGGTGGAATTA
TATTTGATGCAAATATTTGATCACTTAAAACTTTTAAAACTTCTAGGTAATTTGCCACGCTTTTTGACTG
CTCACCAATACCCTGTAAAAATACGTAATTCTTCCTGTTTGTGTAATAAGATATTCATATTTGTAGTTGC
ATTAATAATAGTTATTTCTTAGTCCATCAGATGTTCCCGTGTGCCTCTTTTATGCCAAATTGATTGTCAT
ATTTCATGTTGGGACCAAGTAGTTTGCCCATGGCAAACCTAAATTTATGACCTGCTGAGGCCTCTCAGAA
AACTGAGCATACTAGCAAGACAGCTCTTCTTGAAAAAAAAAATATGTATACACAAATATATACGTATATC
TATATATACGTATGTATATACACACATGTATATTCTTCCTTGATTGTGTAGCTGTCCAAAATAATAACAT
ATATAGAGGGAGCTGTATTCCTTTATACAAATCTGATGGCTCCTGCAGCACTTTTTCCTTCTGAAAATAT
TTACATTTTGCTAACCTAGTTTGTTACTTTAAAAATCAGTTTTGATGAAAGGAGGGAAAAGCAGATGGAC
TTGAAAAAGATCCAAGCTCCTATTAGAAAAGGTATGAAAATCTTTATAGTAAAATTTTTTATAAACTAAA
GTTGTACCTTTTAATATGTAGTAAACTCTCATTTATTTGGGGTTCGCTCTTGGATCTCATCCATCCATTG
TGTTCTCTTTAATGCTGCCTGCCTTTTGAGGCATTCACTGCCCTAGACAATGCCACCAGAGATAGTGGGG
GAAATGCCAGATGAAACCAACTCTTGCTCTCACTAGTTGTCAGCTTCTCTGGATAAGTGACCACAGAAGC
AGGAGTCCTCCTGCTTGGGCATCATTGGGCCAGTTCCTTCTCTTTAAATCAGATTTGTAATGGCTCCCAA
ATTCCATCACATCACATTTAAATTGCAGACAGTGTTTTGCACATCATGTATCTGTTTTGTCCCATAATAT
GCTTTTTACTCCCTGATCCCAGTTTCTGCTGTTGACTCTTCCATTCAGTTTTATTTATTGTGTGTTCTCA
CAGTGACACCATTTGTCCTTTTCTGCAACAACCTTTCCAGCTACTTTTGCCAAATTCTATTTGTCTTCTC
CTTCAAAACATTCTCCTTTGCAGTTCCTCTTCATCTGTGTAGCTGCTCTTTTGTCTCTTAACTTACCATT
CCTATAGTACTTTATGCATCTCTGCTTAGTTCTATTAGTTTTTTGGCCTTGCTCTTCTCCTTGATTTTAA
AATTCCTTCTATAGCTAGAGCTTTTCTTTCTTTCATTCTCTCTTCCTGCAGTGTTTTGCATACATCAGAA
GCTAGGTACATAAGTTAAATGATTGAGAGTTGGCTGTATTTAGATTTATCACTTTTTAATAGGGTGAGCT
TGAGAGTTTTCTTTCTTTCTGTTTTTTTTTTTTGTTTTTTTTTTTTTTTTTTTTTTTTTTTTTTTTGACT
AATTTCACATGCTCTAAAAACCTTCAAAGGTGATTATTTTTCTCCTGGAAACTCCAGGTCCATTCTGTTT
AAATCCCTAAGAATGTCAGAATTAAAATAACAGGGCTATCCCGTAATTGGAAATATTTCTTTTTTCAGGA
TGCTATAGTCAATTTAGTAAGTGACCACCAAATTGTTATTTGCACTAACAAAGCTCAAAACACGATAAGT
TTACTCCTCCATCTCAGTAATAAAAATTAAGCTGTAATCAACCTTCTAGGTTTCTCTTGTCTTAAAATGG
GTATTCAAAAATGGGGATCTGTGGTGTATGTATGGAAACACATACTCCTTAATTTACCTGTTGTTGGAAA
CTGGAGAAATGATTGTCGGGCAACCGTTTATTTTTTATTGTATTTTATTTGGTTGAGGGATTTTTTTATA
AACAGTTTTACTTGTGTCATATTTTAAAATTACTAACTGCCATCACCTGCTGGGGTCCTTTGTTAGGTCA
TTTTCAGTGACTAATAGGGATAATCCAGGTAACTTTGAAGAGATGAGCAGTGAGTGACCAGGCAGTTTTT
CTGCCTTTAGCTTTGACAGTTCTTAATTAAGATCATTGAAGACCAGCTTTCTCATAAATTTCTCTTTTTG
AAAAAAAGAAAGCATTTGTACTAAGCTCCTCTGTAAGACAACATCTTAAATCTTAAAAGTGTTGTTATCA
TGACTGGTGAGAGAAGAAAACATTTTGTTTTTATTAAATGGAGCATTATTTACAAAAAGCCATTGTTGAG
AATTAGATCCCACATCGTATAAATATCTATTAACCATTCTAAATAAAGAGAACTCCAGTGTTGCTATGTG
CAAGATCCTCTCTTGGAGCTTTTTTGCATAGCAATTAAAGGTGTGCTATTTGTCAGTAGCCATTTTTTTG
CAGTGATTTGAAGACCAAAGTTGTTTTACAGCTGTGTTACCGTTAAAGGTTTTTTTTTTTATATGTATTA
AATCAATTTATCACTGTTTAAAGCTTTGAATATCTGCAATCTTTGCCAAGGTACTTTTTTATTTAAAAAA
AAACATAACTTTGTAAATATTACCCTGTAATATTATATATACTTAATAAAACATTTTAAGCTATTTTGTT
GGGCTATTTCTATTGCTGCTACAGCAGACCACAAGCACATTTCTGAAAAATTTAATTTATTAATGTATTT
TTAAGTTGCTTATATTCTAGGTAACAATGTAAAGAATGATTTAAAATATTAATTATGAATTTTTTGAGTA
TAATACCCAATAAGCTTTTAATTAGAGCAGAGTTTTAATTAAAAGTTTTAAATCAGTCCAA

A representative mRNA sequence of CD47 is provided by NCBI Reference Sequence No: NM_001777.4, shown below:

| (SEQ ID NO: 186) | |
|---|---|
| 1 | gcagcctggg cagtgggtcc tgcctgtgac gcgcggcggc ggtcggtcct gcctgtaacg |
| 61 | gcggcggcgg ctgctgctcc ggacacctgc ggcggcggcg gcgaccccgc ggcgggcgcg |
| 121 | gagatgtggc ccctggtagc ggcgctgttg ctgggctcgg cgtgctgcgg atcagctcag |
| 181 | ctactattta ataaaacaaa atctgtagaa ttcacgtttt gtaatgacac tgtcgtcatt |
| 241 | ccatgctttg ttactaatat ggaggcacaa aacactactg aagtatacgt aaagtggaaa |
| 301 | tttaaaggaa gagatattta cacctttgat ggagctctaa acaagtccac tgtccccact |
| 361 | gactttagta gtgcaaaaat tgaagtctca caattactaa aaggagatgc ctctttgaag |
| 421 | atggataaga gtgatgctgt ctcacacaca ggaaactaca cttgtgaagt aacagaatta |
| 481 | accagagaag gtgaaacgat catcgagcta aaatatcgtg ttgtttcatg gttttctcca |
| 541 | aatgaaaata ttcttattgt tattttccca atttttgcta tactcctgtt ctggggacag |
| 601 | tttggtatta aaacacttaa atatagatcc ggtggtatgg atgagaaaac aattgcttta |
| 661 | cttgttgctg gactagtgat cactgtcatt gtcattgttg gagccattct tttcgtccca |
| 721 | ggtgaatatt cattaaagaa tgctactggc cttggtttaa ttgtgacttc tacagggata |
| 781 | ttaatattac ttcactacta tgtgtttagt acagcgattg gattaacctc cttcgtcatt |
| 841 | gccatattgg ttattcaggt gatagcctat atcctcgctg tggttggact gagtctctgt |
| 901 | attgcggcgt gtataccaat gcatggccct cttctgattt caggtttgag tatcttagct |
| 961 | ctagcacaat tacttggact agtttatatg aaatttgtgg cttccaatca gaagactata |
| 1021 | caacctccta ggaaagctgt agaggaaccc cttaatgcat tcaaagaatc aaaaggaatg |
| 1081 | atgaatgatg aataactgaa gtgaagtgat ggactccgat ttggagagta gtaagacgtg |
| 1141 | aaaggaatac acttgtgttt aagcaccatg gccttgatga ttcactgttg gggagaagaa |
| 1201 | acaagaaaag taactggttg tcacctatga gacccttacg tgattgttag ttaagttttt |
| 1261 | attcaaagca gctgtaattt agttaataaa ataattatga tctatgttgt ttgcccaatt |
| 1321 | gagatccagt tttttgttgt tatttttaat caattagggg caatagtaga atggacaatt |
| 1381 | tccaagaatg atgcctttca ggtcctaggg cctctggcct ctaggtaacc agtttaaatt |
| 1441 | ggttcagggt gataactact tagcactgcc ctggtgatta cccagagata tctatgaaaa |
| 1501 | ccagtggctt ccatcaaacc tttgccaact caggttcaca gcagctttgg gcagttatgg |
| 1561 | cagtatggca ttagctgaga ggtgtctgcc acttctgggt caatggaata ataaattaag |
| 1621 | tacaggcagg aatttggttg ggagcatctt gtatgatctc cgtatgatgt gatattgatg |
| 1681 | gagatagtgg tcctcattct tgggggttgc cattcccaca ttcccccttc aacaaacagt |
| 1741 | gtaacaggtc cttcccagat ttagggtact tttattgatg gatatgtttt ccttttattc |
| 1801 | acataacccc ttgaaaccct gtcttgtcct cctgttactt gcttctgctg tacaagatgt |
| 1861 | agcacctttt ctcctctttg aacatggtct agtgacacgg tagcaccagt tgcaggaagg |
| 1921 | agccagactt gttctcagag cactgtgttc acacttttca gcaaaaatag ctatggttgt |
| 1981 | aacatatgta ttcccttcct ctgatttgaa ggcaaaaatc tacagtgttt cttcacttct |
| 2041 | tttctgatct ggggcatgaa aaaagcaaga ttgaaatttg aactatgagt ctcctgcatg |
| 2101 | gcaacaaaat gtgtgtcacc atcaggccaa caggccagcc cttgaatggg gatttattac |
| 2161 | tgttgtatct atgttgcatg ataaacattc atcaccttcc tcctgtagtc ctgcctcgta |
| 2221 | ctccccttcc cctatgattg aaaagtaaac aaaacccaca tttcctatcc tggttagaag |
| 2281 | aaaattaatg ttctgacagt tgtgatcgcc tggagtactt ttagactttt agcattcgtt |
| 2341 | ttttacctgt ttgtggatgt gtgtttgtat gtgcatacgt atgagatagg cacatgcatc |
| 2401 | ttctgtatgg acaaaggtgg ggtacctaca ggagagcaaa ggttaatttt gtgcttttag |
| 2461 | taaaaacatt taaatacaaa gttctttatt gggtggaatt atatttgatg caaatatttg |
| 2521 | atcacttaaa acttttaaaa cttctaggta atttgccacg ctttttgact gctcaccaat |
| 2581 | accctgtaaa aatacgtaat tcttcctgtt tgtgtaataa gatattcata tttgtagttg |
| 2641 | cattaataat agttatttct tagtccatca gatgttcccg tgtgcctctt ttatgccaaa |
| 2701 | ttgattgtca tatttcatgt tgggaccaag tagtttgccc atggcaaacc taaatttatg |
| 2761 | acctgctgag gcctctcaga aaactgagca tactagcaag acagctcttc ttgaaaaaaa |
| 2821 | aaatatgtat acacaaatat atacgtatat ctatatatac gtatgtatat acacacatgt |
| 2881 | atattcttcc ttgattgtgt agctgtccaa aataataaca tatatagagg gagctgtatt |
| 2941 | cctttataca aatctgatgg ctcctgcagc actttttcct tctgaaaata tttacatttt |
| 3001 | gctaacctag tttgttactt taaaaatcag ttttgatgaa aggagggaaa agcagatgga |
| 3061 | cttgaaaaag atccaagctc ctattagaaa aggtatgaaa atctttatag taaaattttt |
| 3121 | tataaactaa agttgtacct tttaatatgt agtaaactct catttatttg gggttcgctc |
| 3181 | ttggatctca tccatccatt gtgttctctt taatgctgcc tgccttttga ggcattcact |
| 3241 | gccctagaca atgccaccag agatagtggg ggaaatgcca gatgaaacca actcttgctc |
| 3301 | tcactagttg tcagcttctc tggataagtg accacagaag caggagtcct cctgcttggg |
| 3361 | catcattggg ccagttcctt ctctttaaat cagatttgta atggctccca aattccatca |
| 3421 | catcacattt aaattgcaga cagtgttttg cacatcatgt atctgttttg tcccataata |
| 3481 | tgctttttac tccctgatcc cagtttctgc tgttgactct tccattcagt tttatttatt |
| 3541 | gtgtgttctc acagtgacac catttgtcct tttctgcaac aacctttcca gctacttttg |
| 3601 | ccaaattcta tttgtcttct ccttcaaaac attctccttt gcagttcctc ttcatctgtg |
| 3661 | tagctgctct tttgtctctt aacttaccat tcctatagta ctttatgcat ctctgcttag |
| 3721 | ttctattagt tttttggcct tgctcttctc cttgatttta aaattccttc tatagctaga |
| 3781 | gcttttcttt ctttcattct ctcttcctgc agtgttttgc atacatcaga agctaggtac |
| 3841 | ataagttaaa tgattgagag ttggctgtat ttagatttat cactttttaa tagggtgagc |
| 3901 | ttgagagttt tctttctttc tgtttttttt ttttgttttt tttttttttt tttttttttt |
| 3961 | tttttttgac taatttcaca tgctctaaaa accttcaaag gtgattattt ttctcctgga |
| 4021 | aactccaggt ccattctgtt taaatcccta agaatgtcag aattaaaata acagggctat |
| 4081 | cccgtaattg gaaatatttc ttttttcagg atgctatagt caatttagta agtgaccacc |
| 4141 | aaattgttat ttgcactaac aaagctcaaa acacgataag tttactcctc catctcagta |
| 4201 | ataaaaatta agctgtaatc aaccttctag gtttctcttg tcttaaaatg ggtattcaaa |
| 4261 | aatggggatc tgtggtgtat gtatggaaac acatactcct taatttacct gttgttggaa |
| 4321 | actggagaaa tgattgtcgg gcaaccgttt attttttatt gtattttatt tggttgaggg |
| 4381 | atttttttat aaacagtttt acttgtgtca tattttaaaa ttactaactg ccatcacctg |
| 4441 | ctggggtcct ttgttaggtc attttcagtg actaataggg ataatccagg taactttgaa |
| 4501 | gagatgagca gtgagtgacc aggcagtttt tctgccttta gctttgacag ttcttaatta |
| 4561 | agatcattga agaccagctt tctcataaat ttctcttttt gaaaaaaaga aagcatttgt |
| 4621 | actaagctcc tctgtaagac aacatcttaa atcttaaaag tgttgttatc atgactggtg |
| 4681 | agagaagaaa acattttgtt tttattaaat ggagcattat ttacaaaaag ccattgttga |
| 4741 | gaattagatc ccacatcgta taaatatcta ttaaccattc taaataaaga gaactccagt |
| 4801 | gttgctatgt gcaagatcct ctcttggagc ttttttgcat agcaattaaa ggtgtgctat |
| 4861 | ttgtcagtag ccattttttt gcagtgattt gaagaccaaa gttgttttac agctgtgtta |
| 4921 | ccgttaaagg tttttttttt tatatgtatt aaatcaattt atcactgttt aaagctttga |
| 4981 | atatctgcaa tctttgccaa ggtacttttt tatttaaaaa aaaacataac tttgtaaata |
| 5041 | ttaccctgta atattatata tacttaataa aacattttaa gctattttgt tgggctattt |
| 5101 | ctattgctgc tacagcagac cacaagcaca tttctgaaaa atttaattta ttaatgtatt |
| 5161 | tttaagttgc ttatattcta ggtaacaatg taaagaatga tttaaaatat taattatgaa |
| 5221 | ttttttgagt ataataccca ataagctttt aattagagca gagttttaat taaaagtttt |
| 5281 | aaatcagtcc aa |

A representative amino acid sequence of CD47 is provided by NCBI Reference Sequence No. NP 001768.1, shown below.

(SEQ ID NO: 187)
MWPLVAALLLGSACCGSAQLLENKTKSVEFTFCNDTVVIPCFVTNMEAQN
TTEVYVKWKFKGRDIYTFDGALNKSTVPTDESSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQKTIQ
PPRKAVEEPLNAFKESKGMMNDE

The present disclosure provides a number of CD34 target sites and corresponding gRNAs that are useful for targeting an RNA-guided nuclease to human CD34. Table 13 below illustrates preferred target domains in the human endogenous CD34 gene that can be bound by gRNAs described herein. The exemplary target sequences of human CD34 shown in Table 13, in some embodiments, are for use with a base editor, e.g., CBE or ABE.

TABLE 13
Exemplary base editor target site sequences of
human CD34 are provided, as are exemplary gRNA
targeting domain sequences useful for targeting
such sites. For each target site, the first
sequence represents the DNA target domain
sequence, the second sequence represents the
reverse complement thereof, and the third
sequence represents an exemplary targeting
domain sequence of a gRNA that can be used
to target the respective target site.

| gRNA Name | Target domain sequence |
|---|---|
| CD34 BE-g12 | TCCCTGGGTAGGTAACTCTG (SEQ ID NO: 120) |
| | CAGAGTTACCTACCCAGGGA (SEQ ID NO: 121) |
| | UCCCUGGGUAGGUAACUCUG (SEQ ID NO: 122) |
| CD34 BE-g11 | TTCCCTGGGTAGGTAACTCT (SEQ ID NO: 123) |
| | AGAGTTACCTACCCAGGGAA (SEQ ID NO: 124) |
| | UUCCCUGGGUAGGUAACUCU (SEQ ID NO: 125) |
| CD34 BE-g10 | GTTCCCTGGGTAGGTAACTC (SEQ ID NO: 126) |
| | GAGTTACCTACCCAGGGAAC (SEQ ID NO: 127) |
| | GUUCCCUGGGUAGGUAACUC (SEQ ID NO: 128) |
| CD34 BE-g9 | TTTGAAAATGTTCCCTGGGT (SEQ ID NO: 129) |
| | ACCCAGGGAACATTTTCAAA (SEQ ID NO: 130) |
| | UUUGAAAAUGUUCCCUGGGU (SEQ ID NO: 131) |
| CD34 BE-g8 | AACATTTGAAAATGTTCCCT (SEQ ID NO: 132) |
| | AGGGAACATTTTCAAATGTT (SEQ ID NO: 133) |
| | AACAUUUGAAAAUGUUCCCU (SEQ ID NO: 134) |
| CD34 BE-g7 | AAACATTTGAAAATGTTCCC (SEQ ID NO: 135) |
| | GGGAACATTTTCAAATGTTT (SEQ ID NO: 136) |
| | AAACAUUUGAAAAUGUUCCC (SEQ ID NO: 137) |
| CD34 BE-g2 | ACCCCAGAGTTACCTACCCA (SEQ ID NO: 138) |
| | TGGGTAGGTAACTCTGGGGT (SEQ ID NO: 139) |
| | ACCCCAGAGUUACCUACCCA (SEQ ID NO: 140) |

A representative DNA sequence of CD34 gene is provided by NCBI Gene ID: 947, shown below.

(SEQ ID NO: 188)
AGTGTCTTCCACTCGGTGCGTCTCTCTAGGAGCCGCGCGGGAAGGATGCTGGTCCGCAGGGGCGCGCGCG
CAGGGCCCAGGATGCCGCGGGGCTGGACCGCGCTTTGCTTGCTGAGTTTGCTGCGTGAGTACCGCCCGCG
CGCCGCGGCCGCTTGGCTTCGCCGCGGGGAGGGTGGAGGCTTTCTGGGAGGCTGAACAGCAGAGCAGAGT
CTCACGGAGGGAAGGGACCCCTGCCCAACCCACGCACTGCCGCCCACAGCTGCTTCCCCCCGGGGCCAGC
GCCTCACCTGGGAGCTGACGGGGGTGGGAGGGGAAGGGAAGGCCATCACCCCCGCGAGTGTGCGTTAGCC
GAGGTGTGAATCGGTCAGCACGACTGGTTCCAATGGACTGAGATAAAGCGCTTTGGAGATGCCAGGGTCT
CTTCTGGTTGCCAGAGGCGCGGAGTGCGAAGTTGCAGCCAGAACCGGGAGACTGGGGAGGAGAGAAGCAG
GAAAAGTTTTGTGGCTCTTGGTTGATTCAGAAAGTATAGACACGGAGCGGATTGCTGGGAAGGGGCCGGT
GTGCCCACCTTGCACAGGGACTGGGAGAGCCAGAAGTGGACATTAAGGAATTCGAGGGAAGCGAATCAGG
GATGAGGCTCCAGGTCCCAGGCCAGGGGTGTTCGGAATGAACAGTCCGTGAAAAGGAAAAACAAAACCAA
ACAACCAACCAAACAAAAACCTTGCATTTAAGATTGGGAAGCTGAGTTTGAATTCCCACTTCAGCAACCC
CACCGCGGAGGAGGTAGAAGTAGACAGAATTTGCTCTGCGCCCAGCCTCGCTCGCCTTACCAAAAACCGT
CACCCCTGCACAAATTTCAAAACTTGTCACCTTTTGGAGTTTTCTTCCTCTGCCCCTGCTCCAAGTTCTA
AAGCTAGTGGATGGGTTAGAACACTGCCCCCACTCACCTACACATATCCTTACTCTTAAGGTCTTGCTCT
CACTTCGGTTAAAAAAAAAATAGGCACCCGGTAAATATTTATACTCGATTACTTTCCTTGGGGAATGACA
TTCTCCACCTCATCTCTTTGCACTTGGAAGGTTTGAAACTTTTGAAACTTTGTACATTGCTCTCCCTTTT
GCTTACAGCACTCTAGGGCTCCCAATTCTGTACTCTCACTACATCCTAAGGGCCACCCACTGTGGCCCTG
CAGAGTGACCTGGACTAGACCTCTTTGTTCCCAGTCTCCTCTGTTGTCCATCTTTGGGTCCGCCCAGCTG
GGGACAGCTGCCCCTGGGCTGGAGGAAGCTCTGAGAGAAGTTTTGGGTTGCCCTGTTTGGCAGTCCTGGA
TGTGTGCTTTGGGGCAAGGAAAAGCTATCAATGAGGTTTCAAAATTCTAGAGTGGGTGCTAGACTAGGCC
CTCATAACAGGAGTGAGTCGGCCGGGCGCAGTGGCTCACGCTTGCAATCCCAGCACTTTGGGAGGCCGAG
GCAGGCAGATCATGAGGTCAAGAGATCGAGACCATCCTGGCCAACATGGTGAAACCCCATCTCTACTAAA
ATTACAAAAATTAGCTGGGCATGGTGGCACACCCCTATAGTCCCAGCTACTCGGGAGGCTGAGGCAGGAG
AATCCCTTGAACCCAGGAGGCGGAGGTTGCAGCGAGCCAAGATCCTGGCACTGCACTCCAGCCTGGGAGA
CAGAGCGAGACTCCTTCTCAAAACAAACAAACAAACAAAAAACAAAAAACAAAAAAACAGGGGTGAGTTG
GCCAGTGGTGCAGATTCCAATCAGATGCTCCATTCTCCTTTGGCTACACAGCTTGTGATGACCTGGTCCT
CAGCCGAATGAGGTCCACCCTTTTCTTATTTCCTCAGCTGGTAAGTGCCAGTCTCGCCTTGGCCTGCCGA
GGTTGGGGTTGGGGGAGCAACACAACCTTGGCAAAACTTTGGACTGATATGCTGAGAATATCCCAGCTTT
TGGTGGGGAGGGATGAGGCACAAGGCCCCATTCCCAGCCTTGCCAGACTACTTGAGCCAATTGTAGTTTT
ATGCCCTTCCCAAGAGCTTTCTAGGGTTTTTGCTTGGATTTGTTTTGTTTTGTTAATACTAGGAGTCATT
TTCAGTCCTGGGATACATTGTGGGATAAATGAGATAACTGAGTGTACCTTGAAATGCTACCATAGGGCCA
TTGCAGAAATGCTATTATTATTGTAAAATTTAGGGTAGAGGGGATTTCTGTGGTTTGTTTTCTCATCATT
TACCCCAACAAAGAGCTTGTTTTGGGAAGTTCTTTTTTTCTGTCCCTTTGAGAAGGTGGCATGTGCATAG
GCACTGCAACTAACCAGAAGTGGGCTGTCTTTGTTGGAAGTGGGGGAGATCACAGAGCAGAGTCAGGGTA
AGGGGCTCAAGGGCCACCAGGCAGCACTGCCAGAGTGCTGGCTGGAGCAGGAAGCCTTTCAGGTAATTAG
GGAACCCCTTTGTCAGGCTCAGGGTATCTATTTCCTCCAAGCTTTCCAAGGCCTGTCCCTATCCTCACTC
TAACAACACCCCCAGGCCCTCCCCGTCACATTCCTCTTCTTCCTTTGGGTCCTTCGTGCCTGCTGACCCC
ACACCCATATCCTGTCCAGGGCTACATGTAAGAGGGGAAAAATAAAAGGGAAAAGAATTTTATCAGCTAT
TTTCCAGAGGGGAGAGGACTCCACCCTTTGAATCTCTGCCATATGGCGACAGAGGAGGAGCAGGCTTGGG
GACCTCTGACTAATTCAGTGGTCCCCAGTGCTCCGCACTTGTAGACCCAACCCAGTTGGCACCACCTTTG
CAGATGCATGTCTTCAGCAGCATTTCTGTTCGGCTGATAAAACACACAGGGGCCTGGGGCCTCTTTTTCT
TATCCCTTTGGCTCCACCTCCTGGGCCTCCTCATTCCCTTGTCCTCATCTCCTTCCTTCGCAATCTTTGT
CTCAAAAAGCTTAGGGCAAGAAGTGCCAAACTGCAGCAGATAAACTCAGCTGCAGAGTCCTACGAAAACA
AATGAAAATGAGGAACAAATTCCACTTCTTACCGTTGGTCTCATTGCCTGATTTCCTTTAGACCCTGAGC
TGGAGGGCCTGGGTTTACTTTCAGGGCTGGATGGTTACCCAACTCCACTCTCAGGTACTTGGGCCTCCCT
CTCTCATTGACTTTGGTTGATCTAACTAAATGGCTTGCAGTGTGGAGAATTAAAAATAAGAAGGTATGGA
AAAAGAACACAACACCACCCTCCAGAAGAAAATTCTCACAGTCGCCAGGGCTGTGAGAATTCCATATTTT
TGTGGGCTTGGACTTCTCTAATGAGAACATTCTCTTCCCTCCTTAGCTAATAAATGAAAACATATTATCT
ATGACTTTTAGTCCCAGAGCTGGACGTTGTGCTAAGGAGAAAGATGCGTGCATGTTGGAAAGATGGAGAG
ACAAACAAATAGGGATGCATATAAAGGTGTACTTCTTAGTACCTGTGCTCTTGGAGTCCGTCGGAATGAG
TTACTTCCCTTCAGATGCAACGGAAAAGGCCTTTGGGCTTAATTGGGAAGTAGGCTTCTTCTTTATTTGG
AAAAATGAAATCAAACTGATACTTCTTAGAGAGGAGTGGCATCCCTAGGCAGCTGATTTAAGAAAATGTC
TTAAGAGGTCTTTGAGTGACAAAGCCTAGAACCCCAGACAATCCTGGATGAGCAAAGGTAGGCAGGAGGC
TGAATTGCTGAGCATGATAAAATGAAGTGAAGCCTTGTGTTTAGGTGGGCACAAGAGACACAAGGCTGAT
AGGACTCCAGAGCCCAGGAAGAGCATATAAAGGGTGGAGAGGTGCCTGGAGTCATAGCAGGACAGTGGTG
GGTGGAGGATGGAAAAGACCTGGGAGGAAATTTTTTTTTAATACATGGCTTCATTAGCACAGTAATGTCT
AATATTTCAGGGTTATGCCATTGTCTCCCCTGGGCTGCCCTGTAATCTCGTTCCATGCCCTCAACCTTTC
AGTAAATACTGGCTACACACACTAGCCTTGTGTTAGTGGCTCTCTGGGGAGGGTAAAGTAACTTACTTTA
CCTTGGCTAACTTACGGTGAAGTGGTATACTAGTGCGGACAGGGAGCCATTGAGCCATGGAGCCACAACC
CAGGAGTCTGTGGTGCTGGTAAAATGGAGGAAGAGGTGACAGGGAGAAATGTAGGTGCCCAGAGAATCTT
CAAAATAATTAGGGTTCCACAGTAAGTGGATGGGGTCCAAGACATGCTATTTTATTTATTTATTTATTTA
TTTATTTATTTGAGAAGAAGTTTCTCTCTTGTTGCTCAGGCTGGAGTACAATGGTGTGATCTCGGATCAC
CGCAACCTCTGCCTCCCAGGTTCAAGCAGTTCTCCTTCCTCAGCCTCCTAAGTAGCTGGGATTACAGGCA
TGCGCCACCATGCCCAGCTAATTTTGTATTTTTAGTAGAGACGGGTTTTCTCTATATTGGTCAGGCTGGT
CTCGAACTCCTGACTTCAGATGATCCACCCACATCGGCCTCCCAAAATGTTGGGATTACAGGCGTGAGCC
ATGGTGTCTGGCCAAGACATGCTATTTGAATATAAAACAGAAATCTCGGTACACTGGGAACCCTCAGGCC
ACAGACCCTTGGTTTATATATACTCACACCTTAGCTTAGCCTTTGAAGCTTAAGAAGGCATCTGCTCAGT
CCCTTGTTCTAGTCTAGGATGACTGAAATTCATGGTAGGCCTCAGAGTTGGACAGCCCCCTAACACGGAT
GCCTCCTGAGTGTTTGCAGGAAGCATGGATGGATCACTTGTCTCCCAAAGTGGCTCATTTCATTCTTATA
AATAGCTACATCATTTTACACTTTTTCCTTGGATTGCAGTGAAATTGGCCTTCCCATAACTTCTAATCAT
TAGTTCTAATTCTATTCTTTGGGCTGGTACAGATTTAATCTGGTCCTTCATGACAATACCTGAAGATGTT
TCTAGTGGGCCCCACTAGATTACAAACTCCTTGAGGGATTGATTTAAATGACAATGACACTAACACCTAA
TGGAGGAAAGAGATCCAGCTCTCATTTTGTACTCTCCACAGCACTGAGCGAGATCCTTTGCACATCACTG
GCACTGCAGAAAAATGAGAATGAATACACAGAATGAATGTATCCCTTATCCAAAATGCTTAGGACCAGAA
GTGTTTCAAATTTCAGATTTTTTCAGATTTTGGAATATCTGTGTAAACATAATGAGATGTCTCAGCAATA
GGACCCAAGTCTAAGCATGAATTTGTGTTTCCTGTATACCTTATATACATAGCCTGAAGATAATTTTAGA
CAATATTTTTAATAAACTTGTGGATGAAACAAAGTGTTGACTGTGTTTTGACTGTGACCTGTCACATGAG
GTCAGGTGTGGAATCTTCCACTTGCGGCATCATGTTGGCACTCAAAAAGTTTCGTATTTTGGAGTATTTT
AGAATTTCTGATTTTCAGATTAGGGGTGCTCAACCAGTGTAAATAACATGCATCTTAAAGAAAGTTATTT
ATTTTTCCTCCCCAAATTCTTCTTTTCCATATCAGGGGTCAGCAAACTTTTTCTATAAAGGGCCAGAGAG
TAAATATTGTAGGCTTTAGGGGCCATATATGGTTTCTGATGGATGGTCTACTTTGTCTTATCTTACGACA
CTTTTAGAAATGTAAAAGCCGGCCACGCACGGTGGCTCATGCCTGCAATCCCAGCACTTCGGGAGGCCGA
GGCAGGCAGATCACCTGAGGTCAGGAGTTTGAGACCAGCTTGGCCAATATGGCTAAACCCCGTCTCTACT
AAAAATACAAAAATTAGCCGGGCATGGTGGCACATGCCTGTAGTCTCAGCTACTTGGGAGGCTGAGGCAG
GAGAATGTCTTGAACCTGGGAGGCAGTGGTTGCAGCCAGCCAAGATCGTGCCACTGCACTCCAGCCTGGG
CAACAGAGTGAGACTGTCTCAAGAAAAAAAGAAAGAAAGGAATGTAAAAGCCATTCTTTACTGACTGACC
ATACAAACACAACCCCTGAGTCAGGTTAGGTTCACTGGCCATAGTTTGCCAACCCCCCATCTAGATGAAA
TAACACTAGACCCTCAACAATATGACTTGGTTTCTAGAACTCTCCTAATCCTGGTTATCTTCACATGAAC
ACTCCCCAATTTAATTAATTCCCCCTTAAAATATAACATGAGAACAAGACTGCCATGACTAGAGGTAGCT
GGCAGGAACACACCTGCCACAGTTGTCCTCCAATCCTATTGCTTTTCCTAAACTCCAAGGATTGTGCTAA
GCACCAGGAGTAAAACACATAAGAATAAGAATGGCTCCAGCACTTTAGAAGTTTATATTCTTACTGAGGT
GACAGTTAGACCTATCCTGAGGAAGCAGCATATATGATTTTGTGGCAGATGAGTCACAGAAATAAGTATC
TCATGCCCACTGAGAAGCAGAAGGAAGGGGCCTAAAACAAAAGATATCCACTATTTCTTGAACTTTTACC
AGACAGGATAACTGCTTCTACTTCTTAGCTCCTTTGTTCCTCAAAACAGCTAAGACATAGGGACTACTTT
TATCTCTGTTTTACAGGTGAGGTTTACAGAGTTGAAGTAACTTCCCAAACCACAGCTAAAACATGGTAAA
GAAGCCTTTGAATGTGGCCTGTCTGGCTGCAGAGCCTGAGCTCTTTGTCTCTGGGCTATACCGCCTTCCT
GGTGATACAGTCTGTGGCTCCCAACGGCCTACTTCCTGGCAGAGGCTGGCATGTCCCTTCATCCTAGTTC
TGTAACTCTATGGACTAAGATTCCCAGGAACTCCTTCGAGATACTCCTCTTGATCCAGGTGCTAGAATAT
GAAAGCATGGGCAAAATAAGACTGGCAGGCTAGGAGATCAGGAAACAGCACCAGGAAAAAACTAAGGAGG
GCTGTGAAAAGCAAGACCAAGTATAGGAAAACTCAGTCTCTTAAAATATGGGGATTTAGTTATTTTGGTA
GCAGAGCTTTCTGGATCGCTGAGGATTTGCAAACTCACTTTAAAGAACAAAAGAGTCTTCAATTTACTAG
TGGCAGGTAGAAATATTTTCAAAATGTATTGCACATGTCTTTGCTTTCTTAGAATACCCCCCCGAAAAGG
ACTTTATGACTCAGATGTATCATGAACATGTCGTGGCTATGTTGCAACTCAGTGACATCATTAATGCTTA
TTAAGGTATAGATATCTAACCTATTCACTCCTACCATAAATGATTCAAGAAAACTGGTGTGAGTTTTCAT
ACTTATGTCTGCTTTTCATTTATTTCTGTTTCTTAGTTTTCAGCTGTTGCTTCTCCTTATTATTTGCTTC
TAATAGGCTCTTCCTACGTTTCAACTTTAGCTAGGATATCAGATAACCAGGGTTATTAGAATTATGTGTA
AAACAAGAATAATTGACTCTGAAGAAGTACCTGAAAGTTCAGTGCATGACTTTTACTACATGAATTTTGT
GACATCTCTTAGAATCCTCATTTTACTAATTTCTTGAGACCTTGTCTCCTCCCTTGCTCCCAGTTCATAA
GTTGAGATCAACTAGAGTAACTAAGGGTCAAATGGAGTCCCTAGGGAAACACAGTTAATAAGGATGGACT
GAGAAGTAAACAACAGAAGAGAGCTTTTTATTCAACTTTAAATTGTACTATAATTCTTGTAGCCTTGTGG
GTAGGGACCGTTCCTCCCATCTAGAATGAAAAAGAGTGGAGGTTTCATCAATTGTCTCTTAGGATGTGAC
AAGCTCTCACTGACTGGTAGGCTCCTGCTACTCCCCTACAGAGTTGGGAGTCAGAAGGTCAGATAAAGAA
AGGTCAGTGTTTGATGGTTTAATGCCATGGATGTGTGTGGGAAGACAGGATAAAAAGAGATCTGTGGTCA
GTCTTGACTCTCATAAATGTCATTCCTCCAGGGCTCTAGAGACTGTGAACCTAAAAGATGCACTCTCCCT
GCTGTGCCGCAGTGTTTATGCCAGGATTGGCTCCAGGCCCCTGGCTAAGGTGTCCCCTTGCTCTGCACCA
GCTCAGGCATTTCCAGTCACTCAGTTTGGTCTTTCTGCCCTCCCACTTGTCCTGTCCCCCTGGTGTCTCA
GAGGGTGCTCTGCTCACCCTGGGGTCAGAGCCCTGACTCTGAAGTTGGGGTCCCTTTCTCATTTGCAGGT
GGATCCTGCTACTCATTCCACAGCGCTCGGCCTGGTGGTGGGTGGAGGGGCTGCTGCAGCTGCGAGCCTG
GCCCCAGTGGGTCAGTAGCTGGCTTGGGTCCAGAGTTGCTTTGCTCTTCTGGAACAGGCACCTCTGTCCT
CAGAACTAGCAGCAGGTCTCACTCCACATCTGGAAAAGTTCAAAAGTGTTTGTCTAGGTGAAGGAGGCCC
TTCGGGGGAGGGGATTCAAAAGCATGACATCATCTCAGTCAGGCCCAGCTTCCTCTGGAGAGAGAAAAGG
GCTGGGCCAGGGTGGACCAGATGGGGTCCCCAGTAATCAGTATGCTGGGGGCGAAGAGGAAGAGAGCAGG
CAAGAAGAGGAGTCTTGGAATGGTGTCTTGAGAAGGTGATCCTTTAATTCAGTGTCTGAATACTGCCCCC
TTGGATGAATTCAGATGAATCTGACCTTAAAGTCAGAGTGGTGAGAGGGTCCCATCTAAACAGGAAAGAA
GCCTGGATTGGCTTCTGCATCAATTTATTTTGATCAACCCACCTCCTGTTTAAGCCAGATGGGAGCCCAT
CCTTTTCTCTACAGAGATGTGGGGAATAAATTCCGATTTTTTGGCAGTTTAGACCCAGCCTAACCTCCTT
GTTGCATCTACTCAAGAGTCAGCTAAACGTCAGTGTTCCATTGCTGCTTCCCTTCATGAACTATGGCCTT
CCTTTTGGGCAGCACCTTGGTGATTCAGGCTCTTCATCTCTAAGACAGAGATAGTGATATTCCTTCTCCC
CACTGCTTACCTGACACATAACAGTAAGACACGATTGCTGCAGTCACCATTAATAGCAATCTGCTTGCAG
CAGGTTGTGGAAGGAAGCAGATATGAGAGAGTGAAACCCTTGTCATCACCTTTAGGGAAACCGTTCTTGG
CCCCAGGTTGATGGCCGTGAAGCCAAAAGTCTAACTTCCCTCTTGATCCTGAGGGAAACAAGAGATGTCA
TTCTACCTTGCTGTTTACCTGGAAATCGCTGAAAAGTACAGATCTAGATCCCAATCAGGAAAATTTAGAG
ACAAGCTTTAGGGTGGCCTGGGACCCTCAGAGGCTCTTCCTTGAAGATGCAACTGCCTTTATGAACTCTG
GATACCAAGTTCACCCTTAACATTTACAAGGCCCAAGGCAAGAGTACAACAGAGGCCTACATACTATATT
TCTAAACATTTCAAGGTTGTAAGTCAGGCCAACTAACTAATAAAATATCTTCTATCCTCTTACCTTGACA
AACATATCTTCGTGATCACCTGGAAGGCCAAGTTTCAATTTGGGATGCTCAGATTCTTGGAATTCACTGC
CACAGTGCAGCTTAGCAGGAAGAGATGGCTCCTGGCCCCAGCCCACTTCCCTTCATCTACTTTCTACCCA
TGACTCTGGCTCACATTGCAAGGCAATTGCATGTGTGCCTGGGACACCCCACCCACAGGCTTAAGCACTA
AGCACTAACTCTTCTCCCTGCAAGCAGTCATCATTTGGCCACCACTGGGCCTTGGGGGTGTACACCCTTG
TGCTGCAGTTCCCCTTAGCAGGATGGACTGGGAGAAGCGGCCCATCGTGGATGTGAGCAAAGTGCCATTT
GATCAAGAAGTGCTAGGACTTCTGGTCCCCACAGTATGACCTAGAAGAGAGGGTACATCCTGATTGGTCC
TCACAGGAAGCAGGACAGGGTGTGGGCTCTTTGAGGCATAGGGTTCAGTCAAGAAGCTTCTCCTTCCTGT
GTCTAATGAGGATACTGACTGGGAATGGTGAGTAACATCTGTAATCCCAGAGCTTTGGGAGGCCAAGACA
GGAGGATCACTTGAGGCCAGGAGCTTAAGGTTACAGTGAGCTATGATCTCACCACTGCATTCTAGCCTGG
GTGCTGTGATGACAGAGCAAGACCCTGTCTTAAAAAAAAAAAAAAAAAAGTATGTATCCCAGGATATACA
GAAGAGATGTGCAAAGACACTCATGAAGATCTTCAGAGAAAAATGAGATCCAAAACATTATACTATAATA
CTATAATAACTTAGCATTTGTTATCTCATTGTTTATCCCAGCAATCCATGTAAGGTAGCCAGAAAAGACC
CTTACTTAAGAGGTAAGAGAGCTGAGATACATACATGCATTAAATTACATGGTTAGTTATTGGAAGAGCC
AGAACAAAGCTTGTCTACCTTCCCACTACATTGATGAGAAACCAGACCTTGTGTTCCCTGGTTCATTAGC
TGAATTCCTAGGTCAGAGCATGCTAAAAGAAGAAAAGGAAAGGCGAATGGGTTTGAAGGCCATGAGCAAA
AGGATGAGGGGAGTATGGGAATGGGAAGTCTGAGAAGTATAGATTTTTAAGGGGATCGCATAGAGACAGT
GAGTCTAGTGATTATTCCAAGAGTTAGTATACTGGATGGAGAGTGACCAAAAAAGAGGATACAGTGAAAA
GAAAAAAGAAGCATGACCTTTTGCACACATTAGATCTTTGCAATATTTATGCAGGTGTAATTTTATATTT
TAGTATGTAACTAAACATACTATGAACATCACCTCATAGTTCCACGTTGCTACAGTTTTTATAATTTTTT
TCTTTCTAATGTTTGTTTATATGATTATATAATGATTATGGTATATTAAGTGCATATATCACTTATCCTA
TCATAATTCATTTAGTTGTTCACGTTTCCCTCCTATAAATAATACTGTATGAGACATTTGTATGCATATA
AATCTTTCCTTCCCTATGTCCTCAGGGCAAATTTGGTAGTGGGAAATTAATCCACTGTCCAGGAGTATGT
TCTTGTTGCTGCCAACAAGGGGCAGCAGGGAATGTTCTAGACGAATTCTACCTGAAGTCAGGAAATTGCA
TCAGGTGATATCACCAGGCTGATATCTTTAGGTTCTGATTCTTCCTCCTCGTTTCCCTATAGCTTCTGGG
TTCATGAGTCTTGACAACAACGGTACTGCTACCCCAGAGTTACCTACCCAGGGAACATTTTCAAATGTTT
CTACAAATGTATCCTACCAAGAAACTACAACACCTAGTACCCTTGGAAGTACCAGCCTGCACCCTGTGTC
TCAACATGGCAATGAGGCCACAACAAACATCACAGGTAAAAACAGCATTTGTGTCAGATCCCGGAGAGAT
GCTGGTGATGCTTGGGTAAAGCATTTAGGATGTTTTCAGACCGCTCCCCTCTCCACAGAGGAAATTATAC
AAGTCCCTAGTATTAATGACTTGAGTATCATGCTTAGGGTGCCCTGAAGTAAGTTCTAGATAATTCTTCC
TTCAGTGACAGTTTTCTCAGGCCCATGTCTTGGGAGCTGATCCTGATCAGTAATGCCTCCATACCCCTTC
TCTCATGCTGAGTCTAGCTTAGTGATAAAATAAGGATCAGAAGACTCAATAGAGATCTCCGATCTCTTCT
AAAGGAAAGAAGGTGGGTACAGGTCCAAACTGGGGGTCTTTGGCTTCTCTAAGGTAGACCAGCATCTATT
TCAGTTTCAGGACACCTCTAATACATTCTAGATTCTAGCTCTTGTTCCAACAGCTTGAAATGAGTTTGGT
CAGGGATGGGACACGAAGTAACTGTTAACTCCCCAAACTCCCTTTTCTGTGTTGAAGTGCATGCCCATAT
CATGACCTGAGATTTTGTGTATCTATGAGCACATGCACACATGCACACCCATGTTTTGGTCTCTTCCAGA
AACGACAGTCAAATTCACATCTACCTCTGTGATAACCTCAGTTTATGGAAACACAAACTCTTCTGTCCAG
TCACAGACCTCTGTAATCAGCACAGTGTTCACCACCCCAGCCAACGTTTCAACTCCAGAGACAACCTTGA
AGCCTAGCCTGTCACCTGGAAATGTTTCAGACCTTTCAACCACTAGCACTAGCCTTGCAACATCTCCCAC
TAAACCCTATACATCATCTTCTCCTATCCTAAGTGACATCAAGGTGGGTGAATTGGGCCAAAAATGGCAG
ATTGCCCCTCACTTCATATGTATGCAGGCAAGCTGTTTCTTTCCCTCCACCCCTCTCCTCATCCCTGCCA
GTGGGATTTGGGTCATGTGGGAATCAGCACGGAAATACACAGTTTAAATATTGCTGGGAGAAGTAGAAAG
AGGAGGAAAGGGGTAGAGTTAGGTGGTAAGGCCCATCCAGGCTTTGGGTATTGCATTTTAGGGAATAGAG
AGTAAACGGGATTCTCAGAGATCCATCCAATCCTCTGGTTCTTTCTAGTACCTATCAGTGGGGCTTTGAC
CAGGACACCATTTCCCTTTGGGAATATTTGGCAACTATTGCTTATTTGCTGGGGCTGCTTCTCCCACAAT
GGTTAGGACAAATAACATTTCCTCTACATGAGAGGGTTTGGTGGTTGTGCCAGGCACAGAGAGGCAGTAA
AGGGGGGCATTGGCAATGGCAGGAACTGGGCAGACCAGGGATGGTGAGCTCAGCCTGGCCTTCTCAGCCT
TAGGGCCTATGACTGTCAACAGCTTTCAGCATGCAGGACAGAAAATATGAGGGCCTCAGTCATGAGATAT
GCCCGAGGGATCTTTCTGCTTTCTGTTTTTAAAGGAGCCAGGGCCAGGCGTGGTGGCTCATGCCTGTAAT
CCCAACACTTTGGGAGCCTGAAGTGGGCAGATCACTTGAGGTCAGGAGTTCAAGACCAGCCCGGGCAACA
TGGTGAAACCCCGTCTCTACCAAAACATACAAAAATTAGCTGGGTGTGGTCATGCACCTGTAGTCCCAGC
TACCCAGGAGGCTGAGGCAAGAGAATCACTTGAACCAGGGAGGTGGAGGTTGCAGTGAGCCAAGATCATG
CCACTGAACTCTAACCTGGGCAAGACTCGATCTCAAAATAAATAAATAAATAAATAAATAAATAAATAAA
TAAAAGAGCCAAAATGGGAATTTGGAGAGTCCTGGAGGCCAGGAGAAAAAACAGGTACCCCAGCATCTGC
TGGTCCACCCCATTATCCCTGTTTGCCTATATGGCCTTCCTGCAAGTTTGTGATTGACTGGAGAGAAAAC
AACCCAAATGGGAAAAGATCCTCCCCTCTCCCAGTCCTGCACCATCCAAGCCAGTGTTTACCAAGGTTGC
AAATAGCTGCTTGCCAGTAAGCCCAGGCTAGTAACAAAAGTTTTGTCATCTGATGAGGATAGATATGAAA
TAGACGAGAGTATGACTATTAGAAATCTCTAGTCCCCTGACAAATTTTAAATGTCCTTTTCCTTAAAACC
TCCTAAGAGAGCACCTCACAGAAAGCAAACTGGAAAAAGTTGGGAAAGAAATGAGGAGCAAAGATACTGG
CTTATTTACTTTGTTTTTGTTATTGTTTTTGTTTTAATACAGGCAGAAATCAAATGTTCAGGCATCAGAG
AAGTGAAATTGACTCAGGGCATCTGCCTGGAGCAAAATAAGACCTCCAGCTGTGTAAGTCAACCCCCCAC
CCAACCTCTTCCTCCCGCCCCTGTCCCTTTCCTCCATCCCTTCTGAACACCCTTAAACCTTCTTGGATTG
CACTGGATTTGAGTAGGGGTCCGGGGAGTTTAGCTTGGTCAGCCTGCCTTTAATACTGAGCTTTCTGTTT
AGGGAGTAAGAGGCCCCATCTGGTGGGCAAAAAAGACAATAACAATTTTAATTATTAATAAATTTTGTTA
TTTTTCCATCCTCTATAGTACTTTTTCCCCCTCCTCTATAGTACTTTTTAAATTTTCCCCTCCTCTGTAG
TACTTTTAAATCTGAGAGAGTCAGATTTAAGTTGCTTTTTGGTTAAGAAAGTCCTTCTCACTCACCAGAT
ATAAACTAGTCACCTAAAATGTCAGTTCATTTATTATTTCATTTATTATTATTAAATATTTACACGTAAC
TCTTTAATCCATATAGAATTTATTTTGCTGTAATGTTATATTTTCCATGTAACCGTTTTTCCTAACATTA
TTTGTTGAATGACTCCCTCTTCTGTTGCATGGGATGTATCCTTTAGCTTATATTAAGTTTTTATATGTCT
TAGGGCATACTCTTCAGACATATTTTATTTGTTCAGTTTGTCTGTACTTTTGCCTATTTTAAATTTTCAA
AATTATTGTATGTTTTTACTACAGTATTTGTAATATCCAGTCAGGCAGATCTTTTTTTATTACACTGTAA
AAAATGTAAATTATTTTTTTCTTTTTGAAAAAAATGCAAAAAAAATGCTTTAAAATTTGAATTGTAATTG
CTCTAAAATTTCTTCATTTTTTCTGGTTTTCTTTTGCATCTCTCAGCATCTTGTTTCTTTAGCTTTTTTC
TCTAGATTTGATGGATGTCATAGGCCTATTTGTCACATAGATCTCCCACTATATATTCAAATTGATTGTC
ACTGATAGATTAAAAAGTTAATAGAGTTTTGAACAATTATTTTGGGTTTGGCCAGTTTCTTGAATTTGCC
TATTGATTCTAGGAGCTTTTTAGTTGATTCCTTTCAATTTTAAGGTAAGCTATCTTGTTATCTACTACAA
TATATTGCTTTTCTCCTCCTGTTTAGTAGTTATGCCTCTTGTTTCAGTTTCATGTCTTACTGCATTGGAA
AGAATGTACAGGGAATTGTGTTATTTTAAATGATTCTGGCAGACCTTGCTTTGTTTCAGTAAATCTAATT
TCCTAACAGTAACATGGCTCTGGAGGAAGGATGAGGTGAAGGAAAGAGTGGTATTTACTTACAGAAGAAA
ACATTTTCTTACAAGTTGGGGATCCATCTAAGGGAATCACATATGTGATGTGCTGGCATGCATAGACTGG
AAAGATCGCAGCCTGTAGAGGGCTTAAATTACTTATTTTTTCCATTCTAACTCGGTAGTTTTACTACGAT
CATCAGTTGGGTCTCTCTGACTTACAAATTTGTAAAAATTATACCTGAAAGAATTTAACTTAGGACATCT
AAAAACAACAGAAATAGTGAAGACATGAGTTTGCATAGTAGAATAACTAAATGCAGTCTGCAAAATAGTT
AACTTAAACATGAACATACAATGTACAAGTAACATGCAGCATGTTTCTGATGCTGATTTTCAACTCAACT
CATTAGAAAAAAAATTGTCAAGTTCTTTGCTAGTTGTAGCCAAGGAAAATGACGATCTTTCCCCCTGCAA
CCCCATCGACTTGGTGGAGCATACATTTTGCTGTGTATCACACAGGTATGTGCCAGAATGCTAATTCTTT
GTGCCTGGAATTTAGAGTTAACTGCAGAGCCTTTGCCTTTCTGTTGCTCTTCAAAGCTCACATGTAACTT
GGGTCACTGTATGAGAAAAGTGCCAGAAACTTACTGCTCTTTTAGAAATAATTCCTCCTAACTCTGCTGT
GGTCTTTGCTGTCTGGCCTACTTCGTGCCACAGCTGAGAAGGTCTCTGCATGTTCCTCTAAGATTGAGGT
AGTATCTGTGAGTGGGGATGTGAAAAGAGAGCAGCTCCCCTCCAGTTCCTATGCCAAAGAAGACTTCCAT
CCACACTGCCCCTTCATAAAGCAACTCTGTGGCAGCTCATATGAGTGCTTTTGTTCCCCTGGTCTCTCCA
CCAGTGTTGATCCCCTTTCTTCCTGAGGGCTCCTCATCCTCAGCCCCCATGCGGCAAAGCCTCCTGCTAC
TCCCCAAGGTAATTTATCCTCAGCTTGCTATCCTGTTCCAAAATGTTTAGCCACAGCTCCCGATTATGTC
CAAGATGACCTTTCCTTGGTCCCTACTCCCTGCTTGTCTGATAAACAATCTGTTCCAAAAGCTGCGAATG
CTGCAGAGCCTCCTAATTTTCCTTAGGTTGGATTTGTACTTTCGGTGTCAGAGGGCCTTAAGGTATTGGT
TCTAATGTTGGGAGCCTCAGGCCCTACCTAATACCCTGGTGAAGAGCAGGAAGGATTTGGTTTCCCCGAG
CATCACATCCTTGATGTATGTTGGTATGCTCCTCATCTAATATATGCCCATAACAGGAATATATGTGCAT
GAGTCTAGCTATCCAGACACTCATTCTGCACTGACACATAGTGAGTACCAATATGTTAGCTCACCCTATG
TGGATTCTGTAGGCGTGTCTTCTGTATGTGCATTCTGTAGACATGTCTATCCCAATCACTCTTTTTTTTT
TGCTCCCCAGCCATCATCTCTAGGCAGCCAGGGGAAGGGAAGGTGATACCACTCTCATCTCAAACACCTT
TTACCATATCTCAGCACCCTCATTAGTAGGATGTGTCTTCCTGAGTCTAACTGTCATTCTTTTGGCAGCA
GTGTCAGCTTAATTTAGTTTTTGTGGTTGTGGTTCCCCAAATAGTTTCCAAAATGTAAGATTACTTTATA
CTATTATGCTACACCCATTTGTCCAGAGAAACCCACCAAAATATTTTTAATTGTGGTAAAATATACATAA
CATAAGTCATCACCTTAACCACTTTTAAGGGTACCATTGAGTAGTAGTAAGTACGTTCACATTATTGTAC
AACCAATCTCTAGAACTGTCTGCATCTTCCATCACTGAAACTGTGTACCCATGAAACAACTCCCTGCTCT
CCCCAGACCCTGGCAACTTTCTGTTCCTATGTTCCTATGACTTTGCCTACTCTAGGTACCTCATATGAGT
AGAGTCATAGTGTTTGTCTTGTTATGTGACTGGTTGATTTCACTCAGTATAATGTCTTTAAAGTCCAACC
ATGTTGTAGCACGTGTCAGAATTTCCTTCCTTTTTAAGGATGAATGATATTCCATTGTAAGTGTAAACCT
TGTTTTGCTTCTCCATTCATCTGTTGATGGACACTTGGGCTTTTTCCCCTTTAGGCTAATGTGAATAATA
CTGCTTTTAACATAAGTGTACAAATATCTCCTTGGCAACCTGATTTGAGTCCTTTTGGGGATATATCCAA
AAGTGAAATGGCTGGACCACATGGGAATTCTATTTTTGAATTTTTGAGGAACCACCAGACCGTTTTACAT
TCCCACCAGTAGGGTACAAGGGTTCCAATTTCTTCTCATCCTCATCAACACGTGTTATTTTCTGGGTTTT
TAAAATAGCAGCCATCCTAATGGGTATGCGGTGGTATTTCATGGTGGTTTTGATTTGCATTTCCCTGATG
ATTAGTGATGTTGAGCCTCTTTCATGTGTTTGTTGGCCATTTGTATATCTTCTTTGGGGAAAAGTCTAGT
CAAGTCCTTTGTCCGCCAATTTGTATACCTATCTTTAGTACCACATCTGTTTATATGCAGAGTTTGTAAT
TACTCAGCTAGCTCACTTTACTCTTATTCACATTGACTTCATCTGAGGCAGGCTGCCTCCAGCTTGCACT
AAAGGTCAGTTTCCATGCGGAAAACATAAGTTACAGATGAAGGATTTATGAAGATGGTGTGTCTACTGGA
AAAAGCCTGCAATCTGGAAACGATCTCACTTCTCATCCTTCGTTAGCTGCCCCCACTTGTTAGGTGACTT
GAAACAGGTCCCTTCATTATTCCATTTTCTATGTGAAAATAGCGTTTTCACCTGCATTGTCAAACTCACA
CCAAAATAGATAAAATTATATTAAAAGGGGTTGGGGGAGATGATTGATCCTAGAATCTCAGCGTTCTAGG
ATCTTAATATCTTAAATATCTTAAAGGTCATTAGAAAAATACCTGTCCCTCCCTCACCCCCATCTCCGTC
CCCATTGCAATGCTTGATGTTAAGAGTCTTTGCAAAGTTTGAGAATGCTACTCTAGAGAGGTTAGATGGG
TTTATTGTGTTTGCAGAGAAACATGTGTGAGCTGATGTAGTTAGGAGCTTGTAAATCTGATTCTCATGAA
CAAAATGGTGAGGCTGTGTCTTCCTCCTGCCCACTCTCCCGTGATGTTTGCCCACAGCCTCTCTTGGGGG
AGGATGTTTATTCCAAGTGTCCGGAGAAGCATTGTGCTTTGTTCAGGATCCACCTGCTCCTCTTGGGGCC
CAAACACAATTCACTGATGCGGTTGGATTCCAGAGTGGGAGGCTGGCCGCTATTATTCCCTTGAATCTAG
TCCAAGAAACTGTGCAAGGGGTGGAGGGACCCTGTGTGGAGGCCAACTGGTTGGGATGTTAAGTTGAACA
ACAATTCCCTGAGTTCTTTTGTTTGGTGCCACGGTTCATGCAGTCTGGGTTGTAAATGCTATAATGTGCA
CTCCCCCCAACACACACTTAGGAGTCTTACAATCTAGTCACAGAAAAATGACAATAACAATGCATAGAAT
ATGATCAATATGTAGACTCTAGATGATATGGTATATAAGGTATTACTATTATTAATAATGATAGTAGCAG
CTGCTATTTATCTAGTGCCCACTAGATTCTCAACACCACATTGGACACTTTTTTTTTTTTGAGACAGAGT
CTTGCTCTGTTGCCCAGGCCGGAGTGCAGAGTGTAGTGTTGTGATCTCAGTTCACTGCAACCTCTGTCTC
CTGGGTTCAAGCGATTCTCGTGCCTCAGCCTGCCGAGTAGCTGGGATTACAGATGCCCACCACCATGCCC
AGCTAATTTTTGCATTTTTTAGAAGAGACAAAGTTTCACCATTTTGGCCAGGCTGGTCTCGAACCCTTGG
CCTCATCTGATTTGTCCATCTCGGCCTCCCAAAGTGCTGGGATTACAGGCATGAGCCACTGCACCCAACT
GGACACTTTCATACAATATCTATTTAGTCACCACAACAACCTAGTGTACAGCTGAGGAACATGACACTCA
GACTAGTTGCATGACTTGCAAAGAATCCAGTAAAGAGCAGAGTAAGGATTGATGCCCAGTCTGGTTGAAG
GTAGGACCAGCTTCATTGAGGAGGTAGAAACTGAGTTGAGTCTTGAAGGATAGTGAATACCAGGGAGAAG
GTAGTGTTCTTCATTCTCTTTGAAATGAGGGTTGTGGAATAATGCATGCTGTCCCCATCTTGGGCTTCCT
GGAGGTGGGGCCTCAGTTCCAAATGCTCTTCCCCACTGGCTTCCTCTGCTCACCCTCCAGCCATTCTTCA
AGGCCTATAGTGACATGTGTCTGCTAACCTTAACCAGACGTGTCCTCACGCTCCTTAGTATGTCCATAGC
ACTTTGGTTGTACCTTTCTTATGGTGCTTACCATGGCAGTAGCTCTGGCCTCTGGAGTCTCTTCACGCCT
CCATTTCTTCATCTACAAAATTAGTAGAATAACTCTACCAATCTCGTAGGTCGTGTGAGGCCTAAATGAG
TTAATATATGGAAAGCACTCAGAACTGTGCCTGGATATCATAGGCATCTGTTACTTTATTTTTTATTTCT
TTATTTTTGAGCCAGGGTCTCTGTCACCCAGGGTGGAGTACAGTGGACTCAACCTCTCAGGGTGGAGAGA
CCCTCCCACCTCAGCCCCCCAAGTAGCTGGTACTATAAGTGCATGCCACCACACCCGGTTGAATTTCTAA
ATTTTTTATAGAGATAGGGTTTCCCTGTGTTGCCCAGGCTGGTCTCGAATTCCTGGGCTGAAGTAATCCT
CCCACCTCAGCCTCCCAAAGTGCTAGGATGACAGGCATGAGTCACCGCGCCTGGTTGGCATCCATCATTT
TAATTGCCGTACATGCAAACTGAAAGCTTTGACAAGGTGGAATTCATAGATACCTCCCAATCCCCACCCC
CCTACACATGACACATGAATACAACACACATACACATGACACTTGCACAGAGGCTTTCACGCAGTGTCCT
ATATTCAGGGCTTGATACATTGTCAAGGTGTGGCTTCTTGCACTCAGCAGGTGGAGGAAGGTGCCATTTG
GTTTGGCTAGTTTACAACCTGCAGGAGCCCTGTCCCTCTGCCTCTCTTGGGAAGCAGACCTATTTCAACT
GGAGATACTTCAGAAGCCCAGAGTCATCCAGGCAAGAAACCCCCTCCTCCTCATCCAGACTCTCGGTGCT
GGGGAGCGGGGGTGCTCAAGGGGAAGCCATGTAAGGCTCCCCTGAGACAACTGGGTTTAGAGAGGTGGAG
ACTGTTGATTGGTTCAGTGTGGCATTCAGACTACTTAGTTCAAATGCTGTTCAGAAAAACGGATTTTTCC
AGAGTTAGAACGTCTATCCAAGGACTTACTGGGAGACCTGCAGAATTGCTCCTTTTCCTGAGGAATGAAG
CAGCAGTGGCCTGAGAACTCATTTCTCTGTAGCCTTGTTTCCTGGGGGTTTTTTGAGGCTCCAGTTTGGG
CTCGTGTCTCTGTGACCTGGAGTTTGGCTAACCACACTCTCCTGGCCTTATCCAAGCCCAGTTGTTTTCC
CTCAGCTGCTTCAAATTCCAGCTGGGTCCTGAGGCCAATCTTGACCTTGCTTTGTGTAGGAGCAAAGGAG
CCTGGGTTTTCCTGCCTTGGGTCACAGCAGTGGGAAAATACCCAGGCTCCATTCCAACTGGGAGGACCCT
GTGGCCTTGTTGCAAGCAGCGGCCCTGCCCGCAAACAGGAAGCTTTCTCCTCCACAGAGACCCAGTTCTG
ATGATGGTCACACACCCCAGCAGTTTTCCCCTAACAGGAAAGTTGTCAGGGCTGTTCAGGCATTTCCTTC
TCTGCCATCTGCCATCCGGACTGAAGAGAAAGTCTTAGTTTCAATTCCTTTCCTGTTCAGGGGAGGAGGA
CACTCTGATTGGAGGCTGCTGGAATAAATTCTGACTCACTATTGAAGAATTATCAGAGTTTTTCTTCTGA
GTCCAAATTCCTGGGTCTGATGGCCCAGTCAGCAACCTGAAGAAAAATCTATGAACTTCATTAAAACTGG
GTTCCATGCTTTTAATGGAAAACAGAGATGGAAGACGAGACAAAAATGCCATTTCAGTTTGAGCAATGCA
CTTTTTGAGACTCCTTTGGGTAGAAAGAGGGAGGAGGCCACTGAGGTTTCCACTGAGTGTATATTATAGA
TTTGTTCTCTCCTCCTCACCTCCCTTGGCTTTTCTTCCATCATTGGGGACTGCTGTAATCTTGAAACAGA
ATATTTTCTCTTAGTTATTATTGCATTATTAATATTTTCTGTTTTTGGCCATTTCTTTTGAGTTTAATTT
GCATATGTGTACATGGGCAATGCTTCAACGAGACAATTTTTTTAAGTTTGTTCTCTAACATCTTAGATAC
TAGAAGATATGGATTTTGGTCCTGGGAGATTATTTAGGTTTTTTTCTTATTGTGTTGTTGCACATATACA
GTTAGCAGTTTTTTTTTTTTCTGTTGGAATTTTAGAAACAAGGATCCTTCTTGCGGACGGGGTAAGAGTA
TGACTGTAATCAGAGACTCTGGGTGCAGTTTAGGGCAGAGCAGTAATTTCATCTCTTGCTCTCCATGTTT
CTCTGTCTTCCCAGGCGGAGTTTAAGAAGGACAGGGGAGAGGGCCTGGCCCGAGTGCTGTGTGGGGAGGA
GCAGGCTGATGCTGATGCTGGGGCCCAGGTATGCTCCCTGCTCCTTGCCCAGTCTGAGGTGAGGCCTCAG
TGTCTACTGCTGGTCTTGGCCAACAGAACAGGTAAGGTGCACCTCTGCCTGGGGGAACAGGGAAGGAGTA
GGGCTGAGGTTAGAGAATCCTGGGTGGAGATGGGGCATCTTAGATCCAGAGAGACCACAGGTGCTGGGGA
GAAGGACTTGGCTGGCTTTGGGAGCGGTCCCCCCGAGATGGACCACCCTGGCCATCAGAGCATCTTCTAG
AACAGCATGGGAGGGTGGAGCAGGATAAGCTGGTTTCTCTTTAGATTTAGCAACCCTTGTTTCTAATTCT
AGAAATTTCCAGCAAACTCCAACTTATGAAAAAGCACCAATCTGACCTGAAAAAGGTAAGTCCTCTGAGA
TGATGGGCCTGAGAGGGAATGCCGGGGCAGGGGGGCTCCCTGGAGTAAGGGGGGAAGTCATTATCTTCCC
CAGTTCTACCTACCATCCTTCTCTGAGTCTTCTCCTTTCTAGTCTTGAACCAAAAATGGTGGGGTATGAC
TGAATTAAGAGACGTCAGTCTCAAATCCCATTTCAAAAATTCCTGAGGTCAATGCCCTCAAGCATCTGTT
CACTGTTGAGTCACACACAGAGGGCTGGAGCGGGCTGGGCTCCACTTTTGGCAAGTGACAGGCTGTATCT
TCTTTGTCTTCTTTTCAAGCTGGGGATCCTAGATTTCACTGAGCAAGATGTTGCAAGCCACCAGAGCTAT
TCCCAAAAGACCCTGATTGCACTGGTCACCTCGGGAGCCCTGCTGGCTGTCTTGGGCATCACTGGCTATT
TCCTGATGAATCGCCGCAGCTGGAGCCCCACAGGAGAAAGGCTGGTCAGTTCTGGGGGCCAGGGTAAAGG
AAATGAGGAAGATAGTGGGTTTCTGGGGAGTTCAGTGGATGTCATGGAGCAGGAGGAGAAATACTAGAAA
AAGCCCTTCTGTGAGCTTACATAAAGATATGCATGTGTGCACACACAGTAATCGGTGGAAGATTCAAAAT
ATTATGTAGCAAACTGGGGAGGGGACAGTAATGGTGCCAGCCCACCTACTCGGTGTGGTAGCAGATGATT
CGTGTCATCTATTCATTATGTTTTCTGGGATAGTTCAGCTTGGGGCCACCAGAACAGGCTGTTTAATCAG
CCACACTGTATTTGCAACCGTGTTAAATGCCCAGTGGGTGCCCCCTTGCTCAAAAGGAGGTATACGGAAG
GAGAATCCCCATTTGCCATTCTGGATGAGGGAGGACAAGGCTGAGGTCTGAATCTTGGCCTCTGGCCTGT
CCCCTACCCTGGGGAGGTCATCCCACCCTTCTTGGAACTGCCCGTTTTCCTGCGGGAGCTGGCTGCCGAG
CTGCGGTGGCATGGTGTGGTGCCTCTCCTATGTCCTTTCTCCTCTAGGAGCTGGAACCCTGACCACTCTT
CAGGAAGAAAGGAGTCTGCACATGCAGCTGCACCCTCCCTCCGATCCTTCCTCCCACCTCCCCCTCCCCC
TTCTCCCACCCCTGCCCCCACTTCCTGTTTGGGCCCCTCTCCCATCCAGTGTCTCACAGCCCTGCTTACC
AGATAATGCTACTTTATTTATACACTGTCTAGGGCGAAGACCCTTATTACACGGAAAACGGTGGAGGCCA
GGGCTATAGCTCAGGACCTGGGACCTCCCCTGAGGCTCAGGGAAAGGCCAGTGTGAACCGAGGGGCTCAG
GAAAACGGGACCGGCCAGGCCACCTCCAGAAACGGCCATTCAGCAAGACAACACGTGGTGGCTGATACCG
AATTGTGACTCGGCTAGGTGGGGCAAGGCTGGGCAGTGTCCGAGAGAGCACCCCTCTCTGCATCTGACCA
CGTGCTACCCCCATGCTGGAGGTGACATCTCTTACGCCCAACCCTTCCCCACTGCACACACCTCAGAGGC
TGTTCTTGGGGCCCTACACCTTGAGGAGGGGCAGGTAAACTCCTGTCCTTTACACATTCGGCTCCCTGGA
GCCAGACTCTGGTCTTCTTTGGGTAAACGTGTGACGGGGGAAAGCCAAGGTCTGGAGAAGCTCCCAGGAA
CAATCGATGGCCTTGCAGCACTCACACAGGACCCCCTTCCCCTACCCCCTCCTCTCTGCCGCAATACAGG
AACCCCCAGGGGAAAGATGAGCTTTTCTAGGCTACAATTTTCTCCCAGGAAGCTTTGATTTTTACCGTTT
CTTCCCTGTATTTTCTTTCTCTACTTTGAGGAAACCAAAGTAACCTTTTGCACCTGCTCTCTTGTAATGA
TATAGCCAGAAAAACGTGTTGCCTTGAACCACTTCCCTCATCTCTCCTCCAAGACACTGTGGACTTGGTC
ACCAGCTCCTCCCTTGTTCTCTAAGTTCCACTGAGCTCCATGTGCCCCCTCTACCATTTGCAGAGTCCTG
CACAGTTTTCTGGCTGGAGCCTAGAACAGGCCTCCCAAGTTTTAGGACAAACAGCTCAGTTCTAGTCTCT
CTGGGGCCACACAGAAACTCTTTTTGGGCTCCTTTTTCTCCCTCTGGATCAAAGTAGGCAGGACCATGGG
ACCAGGTCTTGGAGCTGAGCCTCTCACCTGTACTCTTCCGAAAAATCCTCTTCCTCTGAGGCTGGATCCT
AGCCTTATCCTCTGATCTCCATGGCTTCCTCCTCCCTCCTGCCGACTCCTGGGTTGAGCTGTTGCCTCAG
TCCCCCAACAGATGCTTTTCTGTCTCTGCCTCCCTCACCCTGAGCCCCTTCCTTGCTCTGCACCCCCATA
TGGTCATAGCCCAGATCAGCTCCTAACCCTTATCACCAGCTGCCTCTTCTGTGGGTGACCCAGGTCCTTG
TTTGCTGTTGATTTCTTTCCAGAGGGGTTGAGCAGGGATCCTGGTTTCAATGACGGTTGGAAATAGAAAT
TTCCAGAGAAGAGAGTATTGGGTAGATATTTTTTCTGAATACAAAGTGATGTGTTTAAATACTGCAATTA
AAGTGATACTGAAACACATCTGTTATGTGACTCTGTCTTAGCTGGGTGTGTCTGCATGCAAGAGTGACAC
CCTCCATTAGACCTAGCTAGACTGTGCAGTGATGTGGTGGGGAGGACCAGCCAGGGAAGAGGGAGCACCT
CAGCAGACACAGGCACCAGCCAGGATGCTAAGGACCTTTAGCCAAGTCTGCCAACTATTCTCCTCCATGG
GGAGAGGAAACATCCATTTCCAGTGGTAGAAAGGCAGACCCGAATGTACCAGGGAGCTTCCAAATGGAGG
GTGGTATGTTGGGTTCTTAGGAGCTGTACCCTTCATGAACACCCTTCTGAGAAGAGGAGCATGCTGATCA
CTGCTGCAAAATATGCAAAACAAAGGGAAGGGGCAATGTCCTGTGCACCCTTTATTATCAGGCCACCCCC
CTCCCCAGCCCCCCAGGTCAGAGTAGACACAGTGAAGGACTATGTGGGGACTGTTGTTCTAGAGACCTGG
CAGCCAACTCAGGGAGGGGGCTGGTTTCCACCCTCAAGATTAAGACAGCAGCCTAATTAAAAAAAAAATC
TGTAAGCATGTACCTCCCCCCAGCTTCCAAAACAACCCCCACCCCACCCCTACCAGGCCATAGGAAGTTG
GGGAGGGAGTGCTGAGGAGCTCCAGGAAACACTCCCAAGTGTGTCGACAGTGGCAGAGGCAGTTGGGGCC
AAACAAAGGTTGATTCTTCCATTCTTATCTCCATAAAGCCAGACCTTTCCCTTCAGCACTCCTCCACCCC
CATCTCCTTCTTGCTTTTCTCCAACTCCTCTAATCATAGGTTCTTCCCTAGGACAGAGGGGAGGCGAAAT
GATGAGGTTCAGAGTCTTCCCTCAAAGGCGATGGCTGCCTTGAGGGTTGGAGCAAAGGATGATGAGCAAA
AGACGATGGTAATCAGTAGGGAAGTCCAGCCCACTTGCATCTAGTTGCACATCTTGCCTTGAGAGTAATC
CAGTGAGGGTCTGTCCCAGCTAGGACATCAAGTAGGAGGGGTGGGTTCAGGGTTCAGATTCCTAGGAAAT
ATGGGAGGAGAGGAAAAGGCAACTTGGATGCACCTCCAGCTTCAGGCCTAGCAACCTGCAATGCATCTCA
CCCTGAGTTTGCTGGAATGTGTATGTATGCTTTGGGAGGAAGGGCTGTGTGTGTATTGCGGGGTGGGGTG
GGGCAGCTGGTTCCCTCTGACAGCTGGACAGCTTGCCCTGAAGAATTTGCCTGCTTTCTGGAAAAATCCA
ACTTTCCCACCGTGGGCCTGAGCGTCCTGGTACAGCAATGGCGCCACCTGCTGGCCTTATTGAGGTCCTA
CTGCTCAGCCTCAGCTCAATCGCCTCCATGTTGGGCTTCTCTCCCTGGCTGCCCCACCCTCTAGTCCAAT
TTCTCTTGTACACAAAGCTCATATAACTATAGAACGTCACTGTTGAAGAGAACTTTAAAGATACATTTAA
TTAAACTCCCTTATGGTATAGTTAAAGACAAACTAAGGCTCAGAGAAGGGAGGTGGCTTGCCCAATCACC
CAGAATTCCAAAGTCCTGAATCTGTAGTTTTCCCTTCCATCATATCATCCTACTCTTCTGCCGAGTCCTC
CGTGTTACTCCAGTTGGATGTCATGAAGCCAGTGTGGCAGTGTGAAGATAGGTTTGGGACTTCACTTCTG
GAGCATTTCATCAACATAAGCTATCCTAGGCCTGGCCAGCCAAGCAGGTCCTGGAGGAGCCCCAGGACAA
AGATCACAGGAGGCCATGAGGTTCGGCTTCTTCGGCGCCCACAGTGAGCCCAGGAAAATTAGCTGTAGGG
TATTACACTGTTGACTATGGAGAGCATATCTGGAATTATCTTCAGCCAGATTTTCATCTGAATGGATAAA
TGGGAATACCATCTAAGTCCAGATAAATAGATCACTTCCATCTCATCCCTTCTAGGTAGATTAATCCCAC
ACTTCCTCTTCACACAAAACCAGTAATAGGTCATCGATTTTGTGCAACAGGATGCTGCTTCTCTTCCTAA
AGCCCCCATCGAAGAGGCTTCCAGCCACCATTCAATCATTCATCAAGTCTTATGATGTGCCAGACACTGC
GCGAAATGTGCCAGAACATCTGTTATGTGCCAGACACTGTTCTTGAGACTGGGGATACAGCAAACACTCA
TGAAGCTTATAATTCTAGCAGAAGAGGACAGTAAACAATGTCATCTCAGTAAGTATATACATGTGTTTTC
AGGATTGAGAGCTATGAAAAACATAAAATATATTGAGAATAATGGTTGGTATTTTACATATGGTGGTTAC
TTTTAGAAAAATAACAGTGGAGAGCACAGCTTCACTTGAATGAAGTGGAGAAGCAGGTTGTATGCCAAGC
TGGGAGAGATTATCCCACACAGGGGAAAGGACAAGTGCAAAGCCCTATGATGAAAAGCTGCCAAGTGCAG
AAAGCCTCAGATGGCAGGGGGCAAGATGGCCATGAGGTTGTGTCAGTGAGTGGGGGTGGGGAGAGGCAGG
AGGTCAGACTACATGGGGCCTTTTTAGTTGTAGATTGGGAAGCCACTGGAGGGTTTTGAGCAGAGAAGTC
ATATCATCTGCTTTATGTTTTAAAAGGATCATGCTGGCTGCTGAGTAGAGAATAGAGGTTGAGGGATAAG
AAAGTAGAAGGAGACCGTAGCAAGAAGAACGATCATGGCTGGGAGCAGGTGATCATATTGGCAGTGATGA
GATCAAGCAGAATTCAAAAAGTGGTTTCAAAGTAGAGGTAACAGGACTTGCTCAGTCTATTTATTTCTTC
AAATAATAATCATATTTACAATGATAGTAGCTAACAGTTTTTGAGTGCTTACTGTATGAAAATTGAGATA
TGGTGCCAATATTTAAATAGCATATTTTACTTAACATTCACAGAAACCCTGTGAAGTAGGTTCTATTATC
TCAGAAAAAGAAACTGAAACTCAGAGAATAACAAGGGACTGTGTTACGTGCACAGTGGCAGAGGCAAAGA
TGAATAGGATGTGAGTTTATTTGAACCCCAAATGTTTAAATCTTGGGGATAATACAACACACATTTAAAC
AAAGAAGCAAGAAAAAAAATGCACAACAGAAAGTGAGAAATAACACGAGGAAAGACTAAATGAAGTGCTT
TGTATCTAGATGTGGGCAGGACCCTTTCCAGCTGAGAAGATCTGAGACTGGGTCATGAACAGGTGGTTTC
TGAGTGGGTCCTGTAAAAATGAATACGATTTTGATGATAGTAATGAGTAAGGACATTTGAGACTGATAGA
AGAGTACATACAATATGTAGTGATGGGGAAAGATAAGGTACTGTCAAAGGACAATGTGTTTTCTGGTATG
ACAGAGAAGTAGAATGTGTTAAGGGAAGCCGAGTACCAGAAAGATCCGGGTGTCACAGTTTGTGTAGGGT
GTTTAAAGCTAAACCACAGAGTTTAATTTTATCCAATAGAAGAGGAGCCACAGAAGAGTTTCCATTTATT
CATTAATTTATTCATTTATTCAAAAAATATTTGAGTGCTTATTATAAGCCAGGTACTATGCCAGGCACCT
GGGATAAGACATAGTCCCTTCTGTCAAGTCTTTACATTGGGTGGATGTGGGAGGGACAGATGACAGAACA
ATATGCATTGAGTGTAAGTGCTATGGTATAGGAAGCTCTGAGTGGGAGGGGCATGGAAGCCGTGGAAGAC
CATGGAAGGCTTCCCAGGAGAAGTGACGTCTGGACTGATCCTTTGGTCAAGCAGGAGTTAAAGAGGAGAA
AAGGAGAGATATGGGTGTTCCCGAGAGAGGAAGAAGCCTTGTCCCAGGAGCAAAGTGAGGGTGATTGTTC
CAGAAATGTGAGTGATTCTTTTAAGGCTCAAGCAAAGCATGTGATTCTTCTTTATACCTTCTATTTCTTT
GCTGAGTGTTTCTGTTCTTTTGTTTCAAGCATGCTGCAATTGCTCATTAAAGCATGTTTATGATGGCTGT
CTGTTTTAAAATTCTTGTCAGATGGTTTCAACATCTTTATCATCTCAATGTTGGCATCTGTTAATGGTTT
TTTCTCAATCAAATTGAGATTTTCCTGGTTCTTGGTATTACCAGTGATTTTAATTGCATCTGGAAATTTG
GGATTTATGTTGAAAGACTGGATCTTATTGAAAGATTCTGTTTAGCACCCCTCCTTTGATACCACACTGG
TGGGTCCAGGTTCCCCATTCAGCTGTTGACACCTTCAGGGCAGAGAGGTGGGATGGGGTGAAGGGGGTAC
CTCATTATTGCTGGCCCAGGTTAGAAGTTCAGGCTTCCCAGTAGATCTCTGCTGATACCACCCTGGTGCC
ATGTCATTCCTTGAGTCCAAAAGTCCCTCCCAATTCTGCCTTCTTCTCTCTACATATCGGAGTCTCCCTA
TGTTTGACTTATATATAATGTCCAGGGTTTTTAGAGTTAGTTAACAGGAGGCATAAGAAAAAGTGTGTCC
ACTCCATCTTGTCTGGAACTGGAAGTTCAAGTCGAATATAAGAGAGAGGAGAGGAAATTACAAGCCATGA
GACTGGAGAGTTAGGCAGGTTCTACACCAGCTATTCTCAAAGCCCTCTTACACTCTTAAAAATTTAGAAC
TTCAAAGAGCTTTTGATTTTGAAAGTTACATCTATCAATTATTACTGTTTCAAAAATTAAAATTGAGAAA
ATTTTATTTATTAATTTGTTTAAAAATAACAATAATTATTCAATTACATGATAATGTAAGTAATGCTTTT
CTTAATGAAAAATAATTATATTTTCCAAAACAAAAACAATTAGGAAAAAGAGTGTCATTGTTTTAGACTT
TGGTAAATCTCTCTAATATCTGGCTGAAGAGAAGAATGCTGATTCTTTTTTTTTTTTTTTTTTTTTGAGA
CGGAGTCTCGCTCTGTCACCCAGGCTGGAGTGTAGTGGTGTGATCTCGGCTCACTGCAAGCTCTGCCTCC
CGGGTTCACGCCATTCTCCTGCCTCAGCCTCCCAAGTAGCTGGGACTACAGGCACCCGCCACCACGCCCG
GCTAATTTTTTTGTATTTTTAGTAGAGATGGGGTTTCACCGTGTTAGCCAGGCTGGTCTCGATCTCCTGA
CCTCATGATCCACCCACCTCAGCCTCCCAAAGCGCTGGGATTACAGGTGTGAGACACCGCGCCCAGCCCC
CGAATGCTGATTCTTTTATCTGCTTCTGTATTCAATCTGTTGTGATATGATGGGTAGCCTCTGAAACACT
CCACTGTATACTTGTGAAAGAATGAATGTGAAAAAGGAAAATAGATTTGTAGTATTATTATTCAAATTGT
TTTGACCTCAGAGACCACTTGGAAATGTTTTAGGGAACCCCCAGAGGACCTTGGATCATGCTTTGAGAAC
CGCGGCTCTAGATATGTTACTATTTCAGTAGCATCTAAGTACATGTGGCTGCTGAGCACTTGTAATGTGG
CTAGTGCAAATGAGAGACAGGACTTCCAGCTATATGTAATTTAATAAACTCAAATTTAAAAACTGGAACC
TCATAAAATGTTTTGTTGTTGTTGTTAAACATGACCTTATAGTTTTGGTAGGAA

A representative mRNA sequence of CD34 is provided by NCBI Reference Sequence No: NM_001025109.2, shown below:

| (SEQ ID NO: 189) | |
|---|---|
| 1 | agtgtcttcc actcggtgcg tctctctagg agccgcgcgg gaaggatgct ggtccgcagg |
| 61 | ggcgcgcgcg cagggcccag gatgccgcgg ggctggaccg cgctttgctt gctgagtttg |
| 121 | ctgccttctg ggttcatgag tcttgacaac aacggtactg ctaccccaga gttacctacc |
| 181 | cagggaacat tttcaaatgt ttctacaaat gtatcctacc aagaaactac aacacctagt |
| 241 | acccttggaa gtaccagcct gcaccctgtg tctcaacatg gcaatgaggc cacaacaaac |
| 301 | atcacagaaa cgacagtcaa attcacatct acctctgtga taacctcagt ttatggaaac |
| 361 | acaaactctt ctgtccagtc acagacctct gtaatcagca cagtgttcac caccccagcc |
| 421 | aacgtttcaa ctccagagac aaccttgaag cctagcctgt cacctggaaa tgtttcagac |
| 481 | ctttcaacca ctagcactag ccttgcaaca tctcccacta aaccctatac atcatcttct |
| 541 | cctatcctaa gtgacatcaa ggcagaaatc aaatgttcag gcatcagaga agtgaaattg |
| 601 | actcagggca tctgcctgga gcaaaataag acctccagct gtgcggagtt taagaaggac |
| 661 | aggggagagg gcctggcccg agtgctgtgt ggggaggagc aggctgatgc tgatgctggg |
| 721 | gcccaggtat gctccctgct ccttgcccag tctgaggtga ggcctcagtg tctactgctg |
| 781 | gtcttggcca acagaacaga aatttccagc aaactccaac ttatgaaaaa gcaccaatct |
| 841 | gacctgaaaa agctggggat cctagatttc actgagcaag atgttgcaag ccaccagagc |
| 901 | tattcccaaa agaccctgat tgcactggtc acctcgggag ccctgctggc tgtcttgggc |
| 961 | atcactggct atttcctgat gaatcgccgc agctggagcc ccacaggaga aaggctgggc |
| 1021 | gaagaccctt attacacgga aaacggtgga ggccagggct atagctcagg acctgggacc |
| 1081 | tcccctgagg ctcagggaaa ggccagtgtg aaccgagggg ctcaggaaaa cgggaccggc |
| 1141 | caggccacct ccagaaacgg ccattcagca agacaacacg tggtggctga taccgaattg |
| 1201 | tgactcggct aggtggggca aggctgggca gtgtccgaga gagcacccct ctctgcatct |
| 1261 | gaccacgtgc tacccccatg ctggaggtga catctcttac gcccaaccct tccccactgc |
| 1321 | acacacctca gaggctgttc ttggggccct acaccttgag gaggggcagg taaactcctg |
| 1381 | tcctttacac attcggctcc ctggagccag actctggtct tctttgggta aacgtgtgac |
| 1441 | gggggaaagc caaggtctgg agaagctccc aggaacaatc gatggccttg cagcactcac |
| 1501 | acaggacccc cttcccctac cccctcctct ctgccgcaat acaggaaccc ccaggggaaa |
| 1561 | gatgagcttt tctaggctac aattttctcc caggaagctt tgatttttac cgtttcttcc |
| 1621 | ctgtattttc tttctctact ttgaggaaac caaagtaacc ttttgcacct gctctcttgt |
| 1681 | aatgatatag ccagaaaaac gtgttgcctt gaaccacttc cctcatctct cctccaagac |
| 1741 | actgtggact tggtcaccag ctcctccctt gttctctaag ttccactgag ctccatgtgc |
| 1801 | cccctctacc atttgcagag tcctgcacag ttttctggct ggagcctaga acaggcctcc |
| 1861 | caagttttag gacaaacagc tcagttctag tctctctggg gccacacaga aactcttttt |
| 1921 | gggctccttt ttctccctct ggatcaaagt aggcaggacc atgggaccag gtcttggagc |
| 1981 | tgagcctctc acctgtactc ttccgaaaaa tcctcttcct ctgaggctgg atcctagcct |
| 2041 | tatcctctga tctccatggc ttcctcctcc ctcctgccga ctcctgggtt gagctgttgc |
| 2101 | ctcagtcccc caacagatgc ttttctgtct ctgcctccct caccctgagc cccttccttg |
| 2161 | ctctgcaccc ccatatggtc atagcccaga tcagctccta accettatca ccagctgcct |
| 2221 | cttctgtggg tgacccaggt ccttgtttgc tgttgatttc tttccagagg ggttgagcag |
| 2281 | ggatcctggt ttcaatgacg gttggaaata gaaatttcca gagaagagag tattgggtag |
| 2341 | atattttttc tgaatacaaa gtgatgtgtt taaatactgc aattaaagtg atactgaaac |
| 2401 | acatctgtta tgtgactctg tcttagctgg gtgtgtctgc atgcaagagt gacaccctcc |
| 2461 | attagaccta gctagactgt gcagtgatgt ggtggggagg accagccagg gaagagggag |
| 2521 | cacctcagca gacacaggca ccagccagga tgctaaggac ctttagccaa gtctgccaac |
| 2581 | tattctcctc catggggaga ggaaacatcc atttccagtg gtagaaaggc agacccgaat |
| 2641 | gtaccaggga gcttccaaat ggagggtggt atgttgggtt cttaggagct gtacccttca |
| 2701 | tgaacaccct tctgagaaga ggagcatgct gatcactgct gcaaaatatg caaaacaaag |
| 2761 | ggaaggggca atgtcctgtg caccctttat tatcaggcca cccccctccc cagcccccca |
| 2821 | ggtcagagta gacacagtga aggactatgt ggggactgtt gttctagaga cctggcagcc |
| 2881 | aactcaggga gggggctggt ttccaccctc aagattaaga cagcagccta attaaaaaaa |
| 2941 | aaatctgtaa gcatgtacct ccccccagct tccaaaacaa cccccacccc acccctacca |
| 3001 | ggccatagga agttggggag ggagtgctga ggagctccag gaaacactcc caagtgtgtc |
| 3061 | gacagtggca gaggcagttg gggccaaaca aaggttgatt cttccattct tatctccata |
| 3121 | aagccagacc tttcccttca gcactcctcc acccccatct ccttcttgct tttctccaac |
| 3181 | tcctctaatc ataggttctt ccctaggaca gaggggaggc gaaatgatga ggttcagagt |
| 3241 | cttccctcaa aggcgatggc tgccttgagg gttggagcaa aggatgatga gcaaaagacg |
| 3301 | atggtaatca gtagggaagt ccagcccact tgcatctagt tgcacatctt gccttgagag |
| 3361 | taatccagtg agggtctgtc ccagctagga catcaagtag gaggggtggg ttcagggttc |
| 3421 | agattcctag gaaatatggg aggagaggaa aaggcaactt ggatgcacct ccagcttcag |
| 3481 | gcctagcaac ctgcaatgca tctcaccctg agtttgctgg aatgtgtatg tatgctttgg |
| 3541 | gaggaagggc tgtgtgtgta ttgcggggtg gggtggggca gctggttccc tctgacagct |
| 3601 | ggacagcttg ccctgaagaa tttgcctgct ttctggaaaa atccaacttt cccaccgtgg |
| 3661 | gcctgagcgt cctggtacag caatggcgcc acctgctggc cttattgagg tcctactgct |
| 3721 | cagcctcagc tcaatcgcct ccatgttggg cttctctccc tggctgcccc accctctagt |
| 3781 | ccaatttctc ttgtacacaa agctcatata actatagaac gtcactgttg aagagaactt |
| 3841 | taaagataca tttaattaaa ctcccttatg gtatagttaa agacaaacta aggctcagag |
| 3901 | aagggaggtg gcttgcccaa tcacccagaa ttccaaagtc ctgaatctgt agttttccct |
| 3961 | tccatcatat catcctactc ttctgccgag tcctccgtgt tactccagtt ggatgtcatg |
| 4021 | aagccagtgt ggcagtgtga agataggttt gggacttcac ttctggagca tttcatcaac |
| 4081 | ataagctatc ctaggcctgg ccagccaagc aggtcctgga ggagccccag gacaaagatc |
| 4141 | acaggaggcc atgaggttcg gcttcttcgg cgcccacagt gagcccagga aaattagctg |
| 4201 | tagggtatta cactgttgac tatggagagc atatctggaa ttatcttcag ccagattttc |
| 4261 | atctgaatgg ataaatggga ataccatcta agtccagata aatagatcac ttccatctca |
| 4321 | tcccttctag gtagattaat cccacacttc ctcttcacac aaaaccagta ataggtcatc |
| 4381 | gattttgtgc aacaggatgc tgcttctctt cctaaagccc ccatcgaaga ggcttccagc |
| 4441 | caccattcaa tcattcatca agtcttatga tgtgccagac actgcgcgaa atgtgccaga |
| 4501 | acatctgtta tgtgccagac actgttcttg agactgggga tacagcaaac actcatgaag |
| 4561 | cttataattc tagcagaaga ggacagtaaa caatgtcatc tcagtaagta tatacatgtg |
| 4621 | ttttcaggat tgagagctat gaaaaacata aaatatattg agaataatgg ttggtatttt |
| 4681 | acatatggtg gttactttta gaaaaataac agtggagagc acagcttcac ttgaatgaag |
| 4741 | tggagaagca ggttgtatgc caagctggga gagattatcc cacacagggg aaaggacaag |
| 4801 | tgcaaagccc tatgatgaaa agctgccaag tgcagaaagc ctcagatggc agggggcaag |
| 4861 | atggccatga ggttgtgtca gtgagtgggg gtggggagag gcaggaggtc agactacatg |
| 4921 | gggccttttt agttgtagat tgggaagcca ctggagggtt ttgagcagag aagtcatatc |
| 4981 | atctgcttta tgttttaaaa ggatcatgct ggctgctgag tagagaatag aggttgaggg |
| 5041 | ataagaaagt agaaggagac cgtagcaaga agaacgatca tggctgggag caggtgatca |
| 5101 | tattggcagt gatgagatca agcagaattc aaaaagtggt ttcaaagtag aggtaacagg |
| 5161 | acttgctcag tctatttatt tcttcaaata ataatcatat ttacaatgat agtagctaac |
| 5221 | agtttttgag tgcttactgt atgaaaattg agatatggtg ccaatattta aatagcatat |
| 5281 | tttacttaac attcacagaa accctgtgaa gtaggttcta ttatctcaga aaaagaaact |
| 5341 | gaaactcaga gaataacaag ggactgtgtt acgtgcacag tggcagaggc aaagatgaat |
| 5401 | aggatgtgag tttatttgaa ccccaaatgt ttaaatcttg gggataatac aacacacatt |
| 5461 | taaacaaaga agcaagaaaa aaaatgcaca acagaaagtg agaaataaca cgaggaaaga |
| 5521 | ctaaatgaag tgctttgtat ctagatgtgg gcaggaccct ttccagctga gaagatctga |
| 5581 | gactgggtca tgaacaggtg gtttctgagt gggtcctgta aaaatgaata cgattttgat |
| 5641 | gatagtaatg agtaaggaca tttgagactg atagaagagt acatacaata tgtagtgatg |
| 5701 | gggaaagata aggtactgtc aaaggacaat gtgttttctg gtatgacaga gaagtagaat |
| 5761 | gtgttaaggg aagccgagta ccagaaagat ccgggtgtca cagtttgtgt agggtgttta |
| 5821 | aagctaaacc acagagttta attttatcca atagaagagg agccacagaa gagtttccat |
| 5881 | ttattcatta atttattcat ttattcaaaa aatatttgag tgcttattat aagccaggta |
| 5941 | ctatgccagg cacctgggat aagacatagt cccttctgtc aagtctttac attgggtgga |
| 6001 | tgtgggaggg acagatgaca gaacaatatg cattgagtgt aagtgctatg gtataggaag |
| 6061 | ctctgagtgg gaggggcatg gaagccgtgg aagaccatgg aaggcttccc aggagaagtg |
| 6121 | acgtctggac tgatcctttg gtcaagcagg agttaaagag gagaaaagga gagatatggg |
| 6181 | tgttcccgag agaggaagaa gccttgtccc aggagcaaag tgagggtgat tgttccagaa |
| 6241 | atgtgagtga ttcttttaag gctcaagcaa agcatgtgat tettctttat accttctatt |
| 6301 | tctttgctga gtgtttctgt tcttttgttt caagcatgct gcaattgctc attaaagcat |
| 6361 | gtttatgatg gctgtctgtt ttaaaattct tgtcagatgg tttcaacatc tttatcatct |
| 6421 | caatgttggc atctgttaat ggttttttct caatcaaatt gagattttcc tggttcttgg |
| 6481 | tattaccagt gattttaatt gcatctggaa atttgggatt tatgttgaaa gactggatct |
| 6541 | tattgaaaga ttctgtttag cacccctcct ttgataccac actggtgggt ccaggttccc |
| 6601 | cattcagctg ttgacacctt cagggcagag aggtgggatg gggtgaaggg ggtacctcat |
| 6661 | tattgctggc ccaggttaga agttcaggct tcccagtaga tctctgctga taccaccctg |
| 6721 | gtgccatgtc attccttgag tccaaaagtc cctcccaatt ctgccttctt ctctctacat |
| 6781 | atcggagtct ccctatgttt gacttatata taatgtccag ggtttttaga gttagttaac |
| 6841 | aggaggcata agaaaaagtg tgtccactcc atcttgtctg gaactggaag ttcaagtcga |
| 6901 | atataagaga gaggagagga aattacaagc catgagactg gagagttagg caggttctac |
| 6961 | accagctatt ctcaaagccc tcttacactc ttaaaaattt agaacttcaa agagcttttg |
| 7021 | attttgaaag ttacatctat caattattac tgtttcaaaa attaaaattg agaaaatttt |
| 7081 | atttattaat ttgtttaaaa ataacaataa ttattcaatt acatgataat gtaagtaatg |
| 7141 | cttttcttaa tgaaaaataa ttatattttc caaaacaaaa acaattagga aaaagagtgt |
| 7201 | cattgtttta gactttggta aatctctcta atatctggct gaagagaaga atgctgattc |
| 7261 | tttttttttt tttttttttt tgagacggag tctcgctctg tcacccaggc tggagtgtag |
| 7321 | tggtgtgatc tcggctcact gcaagctctg cctcccgggt tcacgccatt ctcctgcctc |
| 7381 | agcctcccaa gtagctggga ctacaggcac ccgccaccac gcccggctaa tttttttgta |
| 7441 | tttttagtag agatggggtt tcaccgtgtt agccaggctg gtctcgatct cctgacctca |
| 7501 | tgatccaccc acctcagcct cccaaagcgc tgggattaca ggtgtgagac accgcgccca |
| 7561 | gcccccgaat gctgattctt ttatctgctt ctgtattcaa tctgttgtga tatgatgggt |
| 7621 | agcctctgaa acactccact gtatacttgt gaaagaatga atgtgaaaaa ggaaaataga |
| 7681 | tttgtagtat tattattcaa attgttttga cctcagagac cacttggaaa tgttttaggg |
| 7741 | aacccccaga ggaccttgga tcatgctttg agaaccgcgg ctctagatat gttactattt |
| 7801 | cagtagcatc taagtacatg tggctgctga gcacttgtaa tgtggctagt gcaaatgaga |
| 7861 | gacaggactt ccagctatat gtaatttaat aaactcaaat ttaaaaactg gaacctcata |
| 7921 | aaatgttttg ttgttgttgt taaacatgac cttatagttt tggtaggaa |

A representative amino acid sequence of CD34 is provided by NCBI Reference Sequence No. NP_001020280.1, shown below:

(SEQ ID NO: 190)
MLVRRGARAGPRMPRGWTALCLLSLLPSGEMSLDNNGTATPELPTQGTFS
NVSTNVSYQETTTPSTLGSTSLHPVSQHGNEATTNITETTVKFTSTSVIT
SVYGNTNSSVQSQTSVISTVFTTPANVSTPETTLKPSLSPGNVSDLSTTS
TSLATSPTKPYTSSSPILSDIKAEIKCSGIREVKLTQGICLEQNKTSSCA
EFKKDRGEGLARVLCGEEQADADAGAQVCSLLLAQSEVRPQCLLLVLANR
TEISSKLQLMKKHQSDLKKLGILDFTEQDVASHQSYSQKTLIALVTSGAL
LAVLGITGYFLMNRRSWSPTGERLGEDPYYTENGGGQGYSSGPGTSPEAQ
GKASVNRGAQENGTGOATSRNGHSARQHVVADTEL

Some aspects of this disclosure provide genetically engineered cells comprising a modification in their genome that results in expression of a variant form of a lineage-specific cell-surface antigen that is not recognized by an immunotherapeutic agent targeting the lineage-specific cell-surface antigen. In some embodiments, the modification in the genome of the cell is a mutation in a genomic sequence encoding a lineage-specific cell-surface antigen, e.g., in a sequence encoding an epitope bound by an agent that specifically binds the lineage-specific cell-surface antigen. In some embodiments, the modification is effected via genome editing, e.g., using a Cas nuclease and a gRNA targeting a target site encoding an epitope of a lineage-specific cell-surface antigen provided herein or comprising a targeting domain sequence provided herein. In some embodiments, the modification is effected using HDR, e.g., as described herein.

While the compositions, methods, strategies, and treatment modalities provided herein may be applied to any cell or cell type, some exemplary cells and cell types that are particularly suitable for genomic modification in the gene encoding a lineage-specific cell-surface antigen according to aspects of this invention are described in more detail herein. The skilled artisan will understand, however, that the provision of such examples is for the purpose of illustrating some specific embodiments, and additional suitable cells and cell types will be apparent to the skilled artisan based on the present disclosure, which is not limited in this respect.

Some aspects of this disclosure provide genetically engineered hematopoietic cells comprising a modification in their genome that results in expression of a variant form of a lineage-specific cell-surface antigen that is not recognized by an immunotherapeutic agent targeting the lineage-specific cell-surface antigen. In some embodiments, the genetically engineered cells comprising a modification in their genome results in reduced binding by an immunotherapeutic agent targeting the lineage-specific cell-surface antigen, e.g., as compared to a hematopoietic cell (e.g., a hematopoietic stem or progenitor cell, alternatively referred to as “HASPS”) of the same cell type but not comprising a genomic modification. In some embodiments, a hematopoietic cell is a hematopoietic stem cell (HSC). In some embodiments, the hematopoietic cell is a hematopoietic progenitor cell (HPC).

In some embodiments, a hematopoietic cell is a B cell or B cell-committed progenitor cell. As used herein, a B cell-committed progenitor cell is a hematopoietic cell having at least one characteristic of a B cell or B cell lineage cell that precludes it from differentiating into a non-B cell lineage cell (e.g., expression of one or more B cell lineage-specific markers). In some embodiments, a B cell-committed progenitor cell is selected from a Pro-B cell, a Pre-B cell, Immature B cell, or a Mature B cell. In some embodiments, a B cell committed progenitor is a hematopoietic stem cell expressing one or more B cell lineage-specific markers. In some embodiments, a B cell lineage-specific marker is chosen from CD19, CD20, CD34, CD38, CD45, CD45R, or IgM. In some embodiments, a B cell-committed progenitor cell can be engrafted into a subject, wherein the B cell-committed progenitor cell expands and may generate and/or reconstitute cells of the B cell lineage. In some embodiments, a B cell or B cell-committed progenitor cell expresses one or more cell-surface markers, e.g., CD19 and/or CD38. In some embodiments, a genetically engineered cell (e.g., genetically engineered B cell or B cell-committed progenitor cell) described herein expresses a variant cell-surface marker not recognized by an immunotherapeutic agent targeting the cell-surface marker, but nevertheless is capable of expanding and generating and/or reconstituting cells of the B cell lineage.

In some embodiments, the hematopoietic cell is a hematopoietic stem or progenitor cell. As used herein, an HSC refers to a cell capable of self-renewal and which can generate and/or reconstitute all lineages of the hematopoietic system. In some embodiments, an HSC can be engrafted into a subject, wherein the HSC expands and generate and/or reconstitute all lineages of the hematopoietic system. In some embodiments, an HSC expresses one or more cell-surface markers, e.g., CD33, CD123, CD19, CLL-1, CD30, CD5, CD6, CD7, CD34, CD45, CD38, CD47, EMR2/CD312, and BCMA. In some embodiments, a genetically engineered cell (e.g., genetically engineered HSC) described herein expresses a variant cell-surface marker not recognized by an immunotherapeutic agent targeting the cell-surface marker, but nevertheless is capable of self-renewal and can generate and/or reconstitute all lineages of the hematopoietic system.

In some embodiments, a hematopoietic cell (e.g., an HSC or HPC) comprising a modification in their genome that results in expression of a variant form of a lineage-specific cell-surface antigen (e.g., CD19 CD33, CD123, CD19, CLL-1, CD30, CD5, CD6, CD7, CD34, CD38, CD47, EMR2/CD312, and BCMA) that is not recognized by an immunotherapeutic agent targeting the lineage-specific cell-surface antigen, is created using a nuclease and/or a gRNA targeting a lineage-specific cell-surface antigen, and optionally a template polynucleotide, as described herein. In some embodiments, such a cell can be created by contacting the cell with the nuclease and/or the gRNA (and optionally a template polynucleotide), or the cell can be the daughter cell of a cell that was contacted with the nuclease and/or gRNA (and optionally a template polynucleotide). In some embodiments, a cell described herein (e.g., a genetically engineered HSC or HPC) is capable of populating the HSC or HPC niche and/or of reconstituting the hematopoietic system of a subject. In some embodiments, a cell described herein (e.g., an HSC or HPC) is capable of one or more of (e.g., all of): engrafting in a human subject, producing myeloid lineage cells, and producing and lymphoid lineage cells. In some embodiments, a genetically engineered hematopoietic cell provided herein, or its progeny, can differentiate into all blood cell lineages without any differentiation bias as compared to a hematopoietic cell of the same cell type, but not comprising a genomic modification that results in expression of a variant form of a lineage-specific cell-surface antigen that is not recognized by an immunotherapeutic agent targeting the lineage-specific cell-surface antigen. In some embodiments, a genetically engineered hematopoietic cell provided herein, or its progeny, can differentiate into all B cell types and/or exhibits a differentiation bias toward B cells.

In some embodiments, a genetically engineered cell provided herein comprises only one genomic modification, e.g., a genomic modification that results in expression of a variant form of a lineage-specific cell-surface antigen that is not recognized by an immunotherapeutic agent targeting the lineage-specific cell-surface antigen. It will be understood that the gene editing methods provided herein may result in genomic modifications in one or both alleles of a target gene. In some embodiments, genetically engineered cells comprising a genomic modification in both alleles of a given genetic locus are preferred.

In some embodiments, a genetically engineered cell provided herein comprises two or more genomic modifications, e.g., one or more genomic modifications in addition to a genomic modification that results in expression of a variant form of a lineage-specific cell-surface antigen that is not recognized by an immunotherapeutic agent targeting the lineage-specific cell-surface antigen.

In some embodiments, a genetically engineered cell provided herein comprises a genomic modification that results in expression of a variant form of a lineage-specific cell-surface antigen that is not recognized by an immunotherapeutic agent targeting the lineage-specific cell-surface antigen, and further comprises an expression construct that encodes a chimeric antigen receptor, e.g., in the form of an expression construct encoding the CAR integrated in the genome of the cell. In some embodiments, the CAR comprises a binding domain, e.g., an antibody fragment, that binds the lineage-specific cell-surface antigen. In other embodiments, the genetically engineered cell provided herein does not comprise a CAR and/or does not comprise a nucleic acid encoding the CAR.

Some aspects of this disclosure provide genetically engineered immune effector cells comprising a modification in their genome that results in expression of a variant form of a lineage-specific cell-surface antigen that is not recognized by an immunotherapeutic agent targeting the lineage-specific cell-surface antigen. In some embodiments, the immune effector cell is a lymphocyte. In some embodiments, the immune effector cell is a T-lymphocyte. In some embodiments, the T-lymphocyte is an alpha/beta T-lymphocyte. In some embodiments, the T-lymphocyte is a gamma/delta T-lymphocyte. In some embodiments, the immune effector cell is a natural killer T (NKT) cell. In some embodiments, the immune effector cell is a natural killer (NK) cell. In some embodiments, the immune effector cell is a B cell. In some embodiments, the immune effector cell does not express an endogenous transgene, e.g., a transgenic protein. In some embodiments, the immune effector cell expresses a chimeric antigen receptor (CAR). In some embodiments, the immune effector cell expresses a CAR targeting a lineage-specific cell-surface antigen (e.g., CD33, CD123, CD19, CLL-1, CD30, CD5, CD6, CD7, CD34, CD38, CD47, EMR2/CD312, and BCMA). In some embodiments, the immune effector cell does not express a CAR targeting the lineage-specific cell-surface antigen (e.g., does not express a CAR).

In some embodiments, a genetically engineered cell provided herein comprises a genomic modification that results in expression of a variant form of a lineage-specific cell-surface antigen that is not recognized by an immunotherapeutic agent targeting the lineage-specific cell-surface antigen, and does not comprise an expression construct that encodes an exogenous protein, e.g., does not comprise an expression construct encoding a CAR.

In some embodiments, a genetically engineered cell provided herein expresses substantially none of a wild-type lineage-specific cell-surface antigen (e.g., CD33, CD123, CD19, CLL-1, CD30, CD5, CD6, CD7, CD34, CD38, CD47, EMR2/CD312, and BCMA) protein, but expresses a mutant lineage-specific cell-surface antigen protein variant, e.g., a variant not recognized by an immunotherapeutic agent targeting the lineage-specific cell-surface antigen, e.g., a CAR-T cell therapeutic, or an antibody, antibody fragment, or antibody-drug conjugate (ADC) that specifically binds the lineage-specific cell-surface antigen.

In some embodiments, the genetically engineered cells provided herein are hematopoietic cells, e.g., hematopoietic stem cells. Hematopoietic cells are typically characterized by pluripotency, self-renewal properties, and/or the ability to generates cells of the hematopoietic system. In some embodiments, hematopoietic stem cells (HSCs) are capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. In some embodiments, HSCs are characterized by the expression of the cell surface marker CD34 (e.g., CD34+). In some embodiments, CD34 can be used for the identification and/or isolation of HSCs. In some embodiments, HSCs are characterized by lack of expression of one or more cell surface markers (e.g., one or more lineage-specific cell surface markers).

In some embodiments, a genetically engineered HSC disclosed herein (e.g., an HSC that comprises a genetic modification to a gene encoding a lineage-specific cell-surface antigen) can generate a differentiated hematopoietic cell, e.g., a T cell, NK cell, B cell or a progenitor cell of any thereof that expresses a variant of the lineage-specific cell-surface antigen (e.g., not recognized by an immunotherapeutic agent targeting the lineage-specific cell-surface antigen).

In some embodiments, a population of genetically engineered cells described herein comprises a plurality of genetically engineered hematopoietic stem cells. In some embodiments, a population of genetically engineered cells described herein comprises a plurality of genetically engineered hematopoietic progenitor cells. In some embodiments, a population of genetically engineered cells described herein comprises a plurality of genetically engineered hematopoietic stem cells and a plurality of genetically engineered hematopoietic progenitor cells.

In some embodiments, the genetically engineered HSCs are obtained from a subject, such as a human subject. Methods of obtaining HSCs are described, e.g., in International Application No. WO 2017066760, which is herein incorporated by reference in its entirety. In some embodiments, the HSCs are peripheral blood HSCs. In some embodiments, the mammalian subject is a non-human primate, a rodent (e.g., mouse or rat), a bovine, a porcine, an equine, or a domestic animal. In some embodiments, the HSCs are obtained from a human subject, such as a human subject having a hematopoietic malignancy. In some embodiments, the HSCs are obtained from a healthy donor. In some embodiments, the HSCs are obtained from the subject to whom the immune cells expressing the chimeric antigen receptors will be subsequently administered. HSCs that are administered to the same subject from which the cells were obtained are referred to as autologous cells, whereas HSCs that are obtained from a subject who is not the subject to whom the cells will be administered are referred to as allogeneic cells.

In some embodiments, a population of genetically engineered cells is a heterogeneous population of cells, e.g. heterogeneous population of genetically engineered cells containing different lineage-specific cell-surface antigen mutations. In some embodiments, at least 40%, at least 50%, at least 60%, at least 70%, at least 75%, at least 80%, at least 85%, at least 90%, or at least 95% of copies of a gene encoding lineage-specific cell-surface antigen in the population of genetically engineered cells comprise a mutation effected by a genome editing approach described herein, e.g., by a CRISPR/Cas system, base editing using a gRNA provided herein. By way of example, a population of genetically engineered cells can comprise a plurality of different lineage-specific cell-surface antigen mutations (e.g., CD123 mutations, CD38 mutations, CD47 mutations, CD5 mutations, CD34 mutations, EMR2 mutations, or CD19 mutations) and each mutation of the plurality may contribute to the percent of copies of the lineage-specific cell-surface antigen in the population of cells that have a mutation.

In some embodiments, the expression of a lineage-specific cell-surface antigen on the genetically engineered hematopoietic cell (e.g., HSC) is compared to the expression of the lineage-specific cell-surface antigen on a naturally occurring hematopoietic cell (e.g., a wild-type counterpart), e.g., a naturally occurring HSC. In some embodiments, the genetic engineering results in substantially no reduction in the expression level of the lineage-specific cell-surface antigen, or an expression level of at least 50%, at least 60%, at least 70%, at least 80%, at least 90%, at least 91%, at least 92%, at least 93%, at least 94%, at least 95%, at least 96%, at least 97%, at least 98%, or at least 99% of the expression of the lineage-specific cell-surface antigen on a naturally occurring hematopoietic cell (e.g., a wild-type counterpart) or on an otherwise similar cell not containing the genomic modification.

### Methods of Administration to Subjects in Need Thereof

Some aspects of this disclosure provide methods comprising administering an effective number of genetically engineered cells as described herein, comprising a modification in their genome that results in expression of a variant form of a lineage-specific cell-surface antigen that is not recognized by an immunotherapeutic agent targeting the lineage-specific cell-surface antigen, to a subject in need thereof.

A subject in need thereof is, in some embodiments, a subject undergoing or about to undergo an immunotherapy targeting the lineage-specific cell-surface antigen. A subject in need thereof is, in some embodiments, a subject having or having been diagnosed with an autoimmune disease, e.g., characterized by detrimental immune activity of lineage-specific cell-surface antigen-expressing cells. A subject in need thereof is, in some embodiments, a subject having or having been diagnosed with, a malignancy characterized by expression of the lineage-specific cell-surface antigen on malignant cells. In some embodiments, a subject having such a malignancy or autoimmune disease may be a candidate for immunotherapy targeting the lineage-specific cell-surface antigen, but the risk of detrimental on-target, off-disease effects may outweigh the benefit, expected or observed, to the subject. In some such embodiments, administration of genetically engineered cells as described herein, results in an amelioration of the detrimental on-target, off-disease effects, as the genetically engineered cells provided herein are not targeted efficiently by an immunotherapeutic agent targeting the lineage-specific cell-surface antigen.

Examples of autoimmune diseases for which the cells, compositions, and methods described herein may be useful include, without limitation, Achalasia, Addison's disease, Adult Still's disease, Agammaglobulinemia, Alopecia areata, Amyloidosis, Ankylosing spondylitis, Anti-GEM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, or Vogt-Koyanagi-Harada Disease.

In some embodiments, a subject having such a malignancy or autoimmune disease is a candidate for a radiation therapy, e.g., to ablate malignant cells (e.g., lineage-specific cell-surface antigen-expressing malignant cells). In some embodiments, the risk of detrimental off-target effects (e.g., to adjacent or surrounding cells or tissue) and on-target off-disease effects (e.g., to non-malignant lineage-specific cell-surface antigen-expressing cells), may outweigh the benefit, expected or observed, to the subject for radiation therapy. In some embodiments, administration of genetically engineered cells (e.g., genetically engineered hematopoietic cells, e.g., B cells, B cell-committed progenitor cells, or HSCs) described herein after radiation therapy results in an amelioration of the detrimental on-target, off-disease effects. In some embodiments, the combination of an immunotherapeutic approach, e.g., comprising lymphocyte effector cells targeting a lineage-specific cell-surface antigen, such as CAR-T cells or CAR-NK cells, and genetically engineered cells (e.g., genetically engineered stem cells, e.g., HSCs, or genetically engineered B cells or B cell-committed progenitor cells) that express a variant form of the lineage-specific cell-surface antigen that is not recognized by an immunotherapeutic agent targeting the lineage-specific cell-surface antigen is an alternative to radiation therapy for a subject having a lineage-specific cell-surface antigen-expressing malignancy or an autoimmune disease characterized by detrimental immune activity of lineage-specific cell-surface antigen-expressing cells. An immunotherapeutic approach targeting a lineage-specific cell-surface antigen is thought to avoid or significantly decrease the risk of off-target effects (e.g., to adjacent or surrounding cells or tissue). Replenishment of depleted stem cell or differentiated hematopoietic cells (e.g., immune effector cells, B cell, or B cell-committed progenitor cell) populations with immunotherapy-resistant genetically engineered cells (e.g., genetically engineered stem cells, e.g., HSCs) that express a variant form of lineage-specific cell-surface antigen that is not recognized by an immunotherapeutic agent targeting the lineage-specific cell-surface antigen is thought to ameliorate or eliminate on-target off-disease effects of the immunotherapeutic approach.

In some embodiments, the malignancy is a hematologic malignancy, or a cancer of the blood. In some embodiments, the malignancy is a lymphoid malignancy. In general, lymphoid malignancies are associated with the inappropriate production, development, and/or function of lymphoid cells, such as lymphocytes of the T lineage or the B lineage. In some embodiments, the malignancy is characterized or associated with cells that express CD19 on the cell surface.

In some embodiments, the malignancy is associated with aberrant T lymphocytes, such as a T-lineage cancer, e.g., a T cell leukemia or a T-cell lymphoma.

Examples of T cell leukemias and T-cell lymphomas include, without limitation, T-lineage Acute Lymphoblastic Leukemia (T-ALL), Hodgkin's lymphoma, or a non-Hodgkin's lymphoma, acute lymphoblastic leukemia (ALL), chronic lymphocytic leukemia (CLL), large granular lymphocytic leukemia, adult T-cell leukemia/lymphoma (ATLL), T-cell prolymphocytic leukemia (T-PLL), T-cell chronic lymphocytic leukemia, T-prolymphocytic leukemia, T-cell lymphocytic leukemia, peripheral T-cell lymphoma not otherwise specified (PTCL-NOS), enteropathy associated T-cell lymphoma, B-cell chronic lymphocytic leukemia, mantle cell lymphoma, peripheral T-cell lymphoma (PTCL), anaplastic large-cell lymphoma, cutaneous T-cell lymphoma, angioimmunoblastic lymphoma, anaplastic large cell lymphoma, enteropathy-type T-cell lymphoma, hematosplenic gamma-delta T-cell lymphoma, lymphoblastic lymphoma, or hairy cell leukemia.

In some embodiments, the malignancy is associated with aberrant B lymphocytes, such as a B-lineage cancer, e.g., a B-cell leukemia or a B-cell lymphoma. In some embodiments, the malignancy is B-lineage Acute Lymphoblastic Leukemia (B-ALL) or chronic lymphocytic leukemia (B-CLL), primary mediastinal B-cell lymphoma.

In some embodiments, cells of the malignancy express CD33, CD123, CD19, CLL-1, CD30, CD5, CD6, CD7, CD34, CD38, CD47, EMR2/CD312, and/or BCMA, e.g., on their surfaces. In some embodiments, the malignancy comprises a population of cells characterized by expression of a lineage-specific cell-surface antigen, e.g., CD33, CD123, CD19, CLL-1, CD30, CD5, CD6, CD7, CD34, CD38, CD47, EMR2/CD312, and BCMA. In some embodiments, the population of cells characterized by expression of the lineage-specific cell-surface antigen are cancer stem cells. Without wishing to be bound by theory, the cancer stem cell theory suggests that for some malignancies, cancer stem cells share many properties with normal healthy stem cells. In some embodiments, a cancer stem cell expresses the lineage-specific cell-surface antigen, e.g., CD33, CD123, CD19, CLL-1, CD30, CD5, CD6, CD7, CD34, CD38, CD47, EMR2/CD312, and BCMA on its surface. In some embodiments, an immunotherapeutic approach described herein, e.g., comprising lymphocyte effector cells targeting the respective lineage-specific cell-surface antigen, such as CAR-T cells or CAR-NK cells, specifically targets the cancer stem cells of a malignancy. In some embodiments, an immunotherapeutic approach described herein that targets cancer stem cells also has detrimental on-target off-disease effects, e.g., on healthy stem cells, e.g., on non-malignant hematopoietic stem cells, hematopoietic progenitor cells, or lineage-committed blood cells. In some embodiments, genetically engineered cells (e.g., genetically engineered stem cells, e.g., HSCs) that express a variant form of the lineage-specific cell-surface antigen comprising a modified epitope that is not recognized by an immunotherapeutic agent targeting the lineage-specific cell-surface antigen are used to replenish or replace non-cancer stem cells (e.g., healthy stem cells) targeted by the immunotherapeutic approach.

Also within the scope of the present disclosure are malignancies that are considered to be relapsed and/or refractory, such as relapsed or refractory hematological malignancies. A subject in need thereof is, in some embodiments, a subject undergoing or that will undergo an immune effector cell therapy targeting a lineage-specific cell-surface antigen, e.g., CAR-T cell therapy, wherein the immune effector cells express a CAR targeting the lineage-specific cell-surface antigen, and wherein at least a subset of the immune effector cells also express the lineage-specific cell-surface antigen on their cell surface or healthy cells (e.g., stem cells (e.g., HSCs) or endogenous immune effector cells (e.g., B cells)) in the subject undergoing the therapy express the lineage-specific cell-surface antigen on their cell surface.

As used herein, the term “fratricide” refers to self-killing. For example, cells of a population of cells kill or induce killing of cells of the same population. In some embodiments, cells of the immune effector cell therapy kill or induce killing of other cells of the immune effector cell therapy. In such embodiments, fratricide ablates a portion of or the entire population of immune effector cells before a desired clinical outcome, e.g., ablation of malignant cells expressing a lineage-specific cell-surface antigen within the subject, can be achieved. In some such embodiments, using genetically engineered immune effector cells, as provided herein, e.g., immune effector cells that do not express a lineage-specific cell-surface antigen variant recognized by the CAR, as the immune effector cells forming the basis of the immune effector cell therapy, will avoid such fratricide and the associated negative impact on therapy outcome. In such embodiments, genetically engineered immune effector cells, as provided herein, e.g., immune effector cells that do not express a lineage-specific cell-surface antigen variant recognized by the CAR, are be further modified to also express the lineage-specific cell-surface antigen-targeting CAR. In some embodiments, the immune effector cells are lymphocytes, e.g., T-lymphocytes, such as, for example alpha/beta T-lymphocytes, gamma/delta T-lymphocytes, or natural killer T cells. In some embodiments, the immune effector cells are natural killer (NK) cells. In some embodiments, the immune effector cells are B cells.

In some embodiments, cells of the immune effector cell therapy kill or induce killing of stem cells (e.g., HSCs) expressing a lineage-specific cell-surface antigen on their cell surface in the subject. In some embodiments, methods described herein result in depletion of a target stem cell niche (e.g., an HSC niche) in a subject. In some embodiments, methods described herein do not alter or do not appreciably alter the level or viability of stem cells in at least one non-target stem cell niche in a subject. In some embodiments, methods described herein target all stem cell niches of a particular type in a subject (e.g., all HSC niches). In some embodiments, methods described herein result in complete depletion of a stem cell niche (e.g., an HSC niche) in a subject. As used herein, a “stem cell niche” refers to an anatomical area of a subject comprising a specific microenvironment comprising a population of stem cells in an undifferentiated and self-renewable state.

In some embodiments, administering to the subject genetically engineered stem cells expressing a variant form of a lineage-specific cell-surface antigen that is not recognized by an immunotherapeutic agent targeting the lineage-specific cell-surface antigen replenishes the supply of stem cells (e.g., HSCs) in the subject. In some embodiments, a subject is administered a genetically engineered stem cell expressing a variant form of a lineage-specific cell-surface antigen that is not recognized by an immunotherapeutic agent targeting the lineage-specific cell-surface antigen in combination with immune effector cells targeting the lineage-specific cell-surface antigen (e.g., genetically engineered immune effector cells as provided herein, e.g., immune effector cells that do not express a lineage-specific cell-surface antigen variant recognized by the CAR, which are further modified to also express the lineage-specific cell-surface antigen-targeting CAR).

In some embodiments, an effective number of genetically engineered cells as described herein, comprising a modification in their genome that results in expression of a variant form of a lineage-specific cell-surface antigen that is not recognized by an immunotherapeutic agent targeting the lineage-specific cell-surface antigen, is administered to a subject in need thereof, e.g., to a subject undergoing or that will undergo an immunotherapy targeting the lineage-specific cell-surface antigen, wherein the immunotherapy is associated or is at risk of being associated with a detrimental on-target, off-disease effect, e.g., in the form of cytotoxicity towards healthy cells in the subject that express the lineage-specific cell-surface antigen. In some embodiments, an effective number of such genetically engineered cells are administered to the subject in combination with the immunotherapeutic agent targeting the lineage-specific cell-surface antigen.

It is understood that when lineage-specific cell-surface antigen-modified cells (e.g., genetically engineered hematopoietic cells (e.g., stem cells)) and an immunotherapeutic agent targeting the lineage-specific cell-surface antigen) are administered in combination, the cells and the agent may be administered at the same time or at different times, e.g., in temporal proximity. Furthermore, the cells and the agent may be admixed or in separate volumes or dosage forms. For example, in some embodiments, administration in combination includes administration in the same course of treatment, e.g., in the course of treating a subject with an immunotherapy targeting the lineage-specific cell-surface antigen, the subject may be administered an effective number of genetically engineered, lineage-specific cell-surface antigen-modified cells concurrently or sequentially, e.g., before, during, or after the treatment, with the immunotherapy targeting the lineage-specific cell-surface antigen.

In some embodiments, the immunotherapeutic agent that targets a lineage-specific cell-surface antigen as described herein is an immune cell that expresses a chimeric antigen receptor, which comprises an antigen-binding fragment (e.g., a single-chain antibody) capable of binding to the lineage-specific cell-surface antigen. The immune cell is, e.g., a T cell (e.g., a CD4+ or CD8+ T cell) or an NK cell.

A chimeric antigen receptor (CAR) can comprise a recombinant polypeptide comprising at least an extracellular antigen binding domain, a transmembrane domain, and a cytoplasmic signaling domain comprising a functional signaling domain, e.g., one derived from a stimulatory molecule. In some embodiments, the cytoplasmic signaling domain further comprises one or more functional signaling domains derived from at least one costimulatory molecule, such as 4-1BB (i.e., CD137), CD27, and/or CD28, or fragments of those molecules. The extracellular antigen binding domain of the CAR may comprise a lineage-specific cell-surface antigen-binding antibody fragment. The antibody fragment can comprise one or more CDRs, the variable regions (or portions thereof), the constant regions (or portions thereof), or combinations of any of the foregoing.

A chimeric antigen receptor (CAR) typically comprises an antigen-binding domain, e.g., comprising an antibody fragment, fused to a CAR framework, which may comprise a hinge region (e.g., from CD8 or CD28), a transmembrane domain (e.g., from CD8 or CD28), one or more costimulatory domains (e.g., CD28 or 4-1BB), and a signaling domain (e.g., CD3zeta). Exemplary sequences of CAR domains and components are provided, for example in International Publication No. WO 2019/178382, and in Table 14 below, which is incorporated by reference herein in its entirety.

TABLE 14
Exemplary components of a chimeric antigen receptor

| Chimeric antigen receptor component | Amino acid sequence |
|---|---|
| Antigen-binding fragment | Light chain- Linker-Heavy chain |
| CD28 costimulatory domain | IEVMYPPPYLDNEKSNGTI IHVKGKHLCP |
| | SPLFPGPSKPFWVLVVVGGVLACYSLLVTV |
| | AFI I FWVRSKRSRLLHSDYMNMTPRRPGPT |
| | RKHYQPYAPPRDFAAYRS (SEQ ID NO: |
| | 88) |
| CD8 alpha transmembrane domain | IYIWAPLAGTCGVLLLSLVITLYC |
| | (SEQ ID NO: 89) |
| CD28 transmembrane domain | FWVLVVVGGVLACYSLLVTVAFII |
| | FWVRSKRSRLLHSDYMNMTPRR |
| | PGPTRKHYQPYAPPRDFAAYRS |
| | (SEQ ID NO: 90) |
| 4-1BB intracellular domain | KRGRKKLLYI FKQPFMRVQTTQEEDGCS |
| | CRFPEEEEGGCEL (SEQ ID NO: 91) |
| CD3ζ cytoplasmic signaling domain | RVKFSRSADAPAYQQGQNQLYNELNLG |
| | RREEYDVLDKRRGRDPEMGGKPQRRKNP |
| | QEGLYNELQKDKMAEAYSEIGMKGERRR |
| | GKGHDGLYQGLSTATKDTYDALHMQALPPR |
| | (SEQ ID NO: 92) |

In some embodiments, the number of genetically engineered cells provided herein, e.g., HSCs, HPCs, or immune effector cells that are administered to a subject in need thereof, is within the range of 10⁶-10¹¹. However, amounts below or above this exemplary range are also within the scope of the present disclosure. For example, in some embodiments, the number of genetically engineered cells provided herein, e.g., HSCs, HPCs, or immune effector cells that are administered to a subject in need thereof is about 10⁶, about 10⁷, about 10⁸, about 10⁹, about 10¹⁰, or about 10¹¹. In some embodiments, the number of genetically engineered cells provided herein, e.g., HSCs, HPCs, or immune effector cells that are administered to a subject in need thereof, is within the range of 10⁶-10⁹, within the range of 10⁶-10⁸, within the range of 10⁷-10⁹, within the range of about 10⁷-10¹⁰, within the range of 10⁸-10¹⁰, or within the range of 10⁹-10¹¹

In some embodiments, the immunotherapeutic agent that targets a lineage-specific cell-surface antigen is an antibody-drug conjugate (ADC). In some embodiments, the ADC is a molecule comprising an antibody or antigen-binding fragment thereof conjugated to a toxin or drug molecule. Binding of the antibody or fragment thereof to the corresponding antigen allows for delivery of the toxin or drug molecule to a cell that presents the antigen on the its cell surface (e.g., target cell), resulting in death of the target cell.

Suitable antibodies and antibody fragments binding CD19 will be apparent to those of ordinary skill in the art. Examples of anti-CD19 antibodies include, without limitation B43, FMC63, HIB19, 1D3 (e.g., a variant of 1D3, e.g., eBio1D3), SJ25C1, LC1, 60MP31, 771404, OTI3G7, JF100-06, OTI2F6, 6D5, MB19-1, 4G7, 109, OTI3B10, 2E2B6B10, UMAB103, 1C10A1, OTI2B11, OTIIF9, 2E2, JF099-9, OTI1F2, OTI2G7, OTI2D3, J3-129, LT19, SP110, 303, 410, 1G3, 1C9, OTI1E9, HD37, OTI5F3, tafasitamab, loncastuximab, blinatumomab, or CB19 (e.g., as offered in the ThermoFisher Scientific online catalog).

Suitable antibodies and antibody fragments binding CD38 will be apparent to those of ordinary skill in the art. Examples of anti-CD38 antibodies include, without limitation daratumumab, isatuximab, HB7, MIR202, and TAK-079.

Suitable antibodies and antibody fragments binding CD123 will be apparent to those of ordinary skill in the art. Examples of anti-CD123 antibodies include, without limitation, flotetuzumab, vibecotamab, JNJ-63709178, APVO436, 7G3 (JNJ-56022473, or a humanized variant thereof (e.g., antibody CSL-362)), and SAR440234.

Suitable antibodies and antibody fragments binding CD5 will be apparent to those of ordinary skill in the art. Examples of anti-CD5 antibodies include, without limitation, L17F12, AF1636, MB1636, UCHT2, 5D7, CD5/54/F6, LS-C381164, AB-65200, C5/473, OAEE00905, and A58658.

Suitable antibodies and antibody fragments binding CD47 will be apparent to those of ordinary skill in the art. Examples of anti-CD47 antibodies include, without limitation, B6H12, 2D3, SRF231, AF4670, MAB4670, 5F9, Ligufalimab, CC-90002, REA220, LS-C331720, 12283-T26, 66304-1-Ig, 1/1A4, CD47/2937, ADG153, HPAB-0008-FY, 323102, ANC2F6, TA355193, R35991, A00360-1, MEM-122, and D307P.

Suitable antibodies and antibody fragments binding CD34 will be apparent to those of ordinary skill in the art. Examples of anti-CD34 antibodies include, without limitation, QBend10, 561, MAB72271, 581, 8G12, AC136, EP373Y, CBL496-25UG, MEC 14.7, 4H11, and 43A1.

Suitable antibodies and antibody fragments binding EMR2 will be apparent to those of ordinary skill in the art, and include, for example, those described in PCT Publication No. WO2017/087800, the entire contents of which are incorporated herein by reference.

In some embodiments, the agent that specifically binds the lineage-specific cell-surface antigen is an antibody-drug conjugate. Toxins or drugs compatible for use in antibody-drug conjugates are known in the art and will be evident to one of ordinary skill in the art. See, e.g., Peters et al. Biosci. Rep. (2015) 35 (4): e00225; Beck et al. Nature Reviews Drug Discovery (2017) 16:315-337; Marin-Acevedo et al. J. Hematol. Oncol. (2018) 11:8; Elgundi et al. Advanced Drug Delivery Reviews (2017) 122:2-19.

In some embodiments, the antibody-drug conjugate further comprises a linker (e.g., a peptide linker, such as a cleavable linker) attaching the antibody and the drug molecule.

Examples of suitable toxins or drugs for antibody-drug conjugates include, without limitation, the toxins and drugs comprised in brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIVIA, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab vedotin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BIIB015, MLN2704, AMG 172, AMG 595, LOP 628, vadastuximab talirine/SGN-CD33A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-107, MEDI4276, DSTA4637S/RG7861.

In some embodiments, binding of the antibody-drug conjugate to the epitope of the cell-surface lineage-specific protein induces internalization of the antibody-drug conjugate, and the drug (or toxin) may be released intracellularly. In some embodiments, binding of the antibody-drug conjugate to the epitope of a cell-surface lineage-specific protein induces internalization of the toxin or drug, which allows the toxin or drug to kill the cells expressing the lineage-specific protein (target cells). In some embodiments, binding of the antibody-drug conjugate to the epitope of a cell-surface lineage-specific protein induces internalization of the toxin or drug, which may regulate the activity of the cell expressing the lineage-specific protein (target cells). The type of toxin or drug used in the antibody-drug conjugates described herein is not limited to any specific type.

### Homology-Directed Repair (HDR) Using Template Polynucleotides

In some embodiments, the present disclosure provides genetically engineered cells and cell populations, and methods of producing genetically engineered cells and cell populations using HDR-mediated gene editing, e.g., CRISPR/Cas-based HDR-mediated gene editing. Without being bound by any particular theory, HDR is a process wherein damage to DNA (e.g., a break in the DNA) is repaired using a donor sequence with flanking sequences comprising homology to the site of DNA damage. In some embodiments, a CRISPR/Cas system is used to introduce a break in the DNA (e.g., a double-stranded break (DSB)). In some embodiments, by providing a donor sequence (e.g., via a template polynucleotide) in the presence of a DSB, HDR is promoted (e.g., relative to other DNA repair pathways, e.g., NHEJ). In some embodiments, HDR results in substitution or insertion mutations that replace endogenous or naturally occurring sequences with those of the donor sequence. In some embodiments, methods described herein are used to introduce a mutation into a gene encoding a lineage-specific cell-surface antigen, e.g., to modify an epitope bound by an agent that specifically binds the lineage-specific cell-surface antigen.

In some embodiments, the donor sequence is provided by, for example, a template polynucleotide. When the donor sequence differs at one or more positions relative to a gene encoding a lineage-specific cell-surface antigen, integration of the donor sequence by HDR results in a mutation. In some embodiments, a donor sequence differs from a sequence in the gene encoding a lineage-specific cell-surface antigen in one or more nucleotides, and integration of the donor sequence into the gene encoding a lineage-specific cell-surface antigen produces a genetic modification in the gene encoding a lineage-specific cell-surface antigen. In some embodiments, the donor sequence differs from a gene encoding a lineage-specific cell-surface antigen in a manner that integration of the donor sequence alters the amino acid sequence of an epitope of a lineage-specific cell-surface (e.g., an epitope bound by an agent that specifically binds the lineage-specific cell-surface antigen). In some embodiments, the donor sequence differs from the sequence of a gene encoding a lineage-specific cell-surface antigen such that integration of the donor sequence introduces one or more silent mutations in addition to altering the amino acid sequence of an epitope.

In some embodiments, a template polynucleotide is single-stranded, e.g., a single-strand donor oligonucleotide (ssODN). In some embodiments, a template polynucleotide is double-stranded, e.g., a plasmid or a double-stranded donor oligonucleotide (dsODN). As used herein, a template polynucleotide refers to a nucleic acid that is a template for HDR, e.g., HDR of a mutation in the gene encoding a lineage-specific cell-surface antigen. In some embodiments, a template polynucleotide is approximately 10, 20, 30, 40, 50, 60, 70, 80, 90, 100, 110, 120, 130, 140, 150, 160, 170, 180, 190, or 200 nucleotides long, +/−1, 2, 3, 4, 5, 6, 7, 8, 9, or 10 nucleotides long.

In some embodiments, the donor sequence comprises a modification as compared to the gene encoding a lineage-specific cell-surface antigen, for example, a mutation, e.g., an insertion, deletion, or substitution as compared to the gene encoding a lineage-specific cell-surface antigen nucleotide sequence. In some embodiments, the donor sequence comprises a substitution of a single nucleotide as compared to the gene encoding a lineage-specific cell-surface antigen. Such donor sequences are useful, for example, to effect genetic modifications that alter a single nucleotide, e.g., changing a codon to encode a different amino acid, in a gene encoding a lineage-specific cell-surface antigen sequence encoding an epitope (e.g., bound by an agent that specifically binds to a lineage-specific cell-surface antigen). In some embodiments, the donor sequence comprises a substitution of two or more nucleotides as compared to the gene encoding a lineage-specific cell-surface antigen. Such donor sequences are useful, for example, to effect genetic modifications that alter, e.g., multiple codons, in a gene encoding a lineage-specific cell-surface antigen sequence encoding an epitope (e.g., bound by an agent that specifically binds to a lineage-specific cell-surface antigen). In some embodiments, the donor sequence comprises one or more insertions (e.g., of one or more nucleotides) as compared to the gene encoding a lineage-specific cell-surface antigen. Such donor sequences are useful, for example, to effect genetic modifications that create insertion mutations in a gene encoding a lineage-specific cell-surface antigen sequence encoding an epitope (e.g., bound by an agent that specifically binds to a lineage-specific cell-surface antigen). In some embodiments, the donor sequence comprises one or more deletions (e.g., of one or more nucleotides) as compared to the gene encoding a lineage-specific cell-surface antigen. Such donor sequences are useful, for example, to effect genetic modifications that create deletion mutations in a gene encoding a lineage-specific cell-surface antigen sequence encoding an epitope (e.g., bound by an agent that specifically binds to a lineage-specific cell-surface antigen). In some embodiments, the donor sequence comprises two or more substitutions as compared to the gene encoding a lineage-specific cell-surface antigen, wherein, if integrated into the gene encoding a lineage-specific cell-surface antigen, at least one such substitution results in an amino acid change to an epitope (e.g., bound by an agent that specifically binds to a lineage-specific cell-surface antigen) and optionally wherein at least one such substitution results in a silent mutation in the gene encoding a lineage-specific cell-surface antigen, e.g., a substitution of a wobble base within an amino acid-encoding codon of a gene encoding a lineage-specific cell-surface antigen. Such donor sequences are useful, for example, to effect genetic modifications that disrupt binding of an agent to the lineage-specific cell-surface antigen, while at the same time creating a sequence tag, e.g., a non-naturally occurring sequence or a sequence that was not previously present in the gene encoding a lineage-specific cell-surface antigen, which is useful for identification and/or tracking of the modified cells. In some embodiments, the donor sequence comprises a restriction site or a unique sequence tag, for example, a unique primer binding site. In some embodiments, the sequence comprising the restriction site or a unique sequence tag is an insertion relative to the gene encoding a lineage-specific cell-surface antigen e.g., the gene encoding a lineage-specific cell-surface antigen does not comprise a restriction site or a unique sequence tag where the donor sequence comprises one. In some embodiments, the sequence comprising the restriction site or a unique sequence tag is not an insertion relative to the gene encoding a lineage-specific cell-surface antigen. For example, in some embodiments, the sequence comprising the restriction site or a unique sequence tag comprises a mutation (e.g., a substitution) as compared to the gene encoding a lineage-specific cell-surface antigen that, upon integration into the gene encoding a lineage-specific cell-surface antigen, produces a restriction site or a unique sequence tag. In some embodiments, the sequence comprising the restriction site or a unique sequence tag does not alter an amino acid sequence encoded by the gene encoding a lineage-specific cell-surface antigen. In some embodiments, restriction site or a unique sequence tag introduced in such a manner is used as a tag or “barcode”, e.g., to confirm the success of integration of the donor sequence (e.g., in an experiment where the modified gene encoding a lineage-specific cell-surface antigen, such as a sequence encoding an epitope bound by an agent that specifically binds the lineage-specific cell-surface antigen is cleaved and fragments or sequences thereof are analyzed). In some embodiments, the restriction endonuclease site comprises a Pvul site, e.g., 5′-CGATCG-3′.

In some embodiments, the donor sequence differs from the gene encoding a lineage-specific cell-surface antigen in a manner such that integration of the donor sequence alters the amino acid sequence of an epitope bound by an agent that specifically binds the lineage-specific cell-surface antigen and produces one or more additional mutations (e.g., a second, third, fourth, or fifth mutation relative to the epitope modification (the first mutation)). In some embodiments, the one or more additional mutations comprise one or more silent mutations that do not alter the amino acid encoded by the nucleic acid sequence of the gene encoding a lineage-specific cell-surface antigen. In some embodiments, the one or more silent mutations are contiguous (i.e., directly adjacent) to the amino acid encoding sequence modification. In some embodiments, silent mutations are used, e.g., as identifiers (e.g., “tags” or “bar codes”) of a amino acid alteration or to facilitate confirmation of integration of the donor sequence (e.g., in an experiment where the modified gene encoding a lineage-specific cell-surface antigen sequences, such as a sequence encoding an epitope bound by an agent that specifically binds the lineage-specific cell-surface antigen, are analyzed).

In some embodiments, methods and compositions provided by the present disclosure are applied to a gene encoding a lineage-specific cell-surface antigen, e.g., in order to modify the gene encoding the lineage-specific cell-surface antigen such as a sequence encoding an epitope bound by an agent that specifically binds the lineage-specific cell-surface antigen. For example, in some embodiments, the gene encoding a lineage-specific cell-surface antigen comprises a nucleotide sequence that encodes a lineage-specific cell-surface antigen, e.g., an epitope of the lineage-specific cell-surface antigen that is bound by an agent that specifically binds the lineage-specific cell-surface antigen.

As used herein, a gene encoding a lineage-specific cell-surface antigen (e.g., a sequence encoding an epitope bound by an agent that specifically binds the lineage-specific cell-surface antigen) refers to any nucleic acid in which a break (e.g., a double-stranded break (DSB)) is targeted (e.g., by a CRISPR/Cas system). In some embodiments, a DSB in a gene encoding a lineage-specific cell-surface antigen (e.g., a sequence encoding an epitope bound by an agent that specifically binds the lineage-specific cell-surface antigen) can be repaired by HDR. In some embodiments, the gene encoding a lineage-specific cell-surface antigen (e.g., a sequence encoding an epitope bound by an agent that specifically binds the lineage-specific cell-surface antigen) is a genomic nucleic acid sequence, e.g., in a cell, e.g., in a subject, e.g., a human subject. In some embodiments, the gene encoding a lineage-specific cell-surface antigen (e.g., a sequence encoding an epitope bound by an agent that specifically binds the lineage-specific cell-surface antigen) comprises a gene or a portion thereof (e.g., a coding portion thereof, e.g., an exon). In some embodiments, the gene encoding a lineage-specific cell-surface antigen (e.g., a sequence encoding an epitope bound by an agent that specifically binds the lineage-specific cell-surface antigen) comprises a non-coding portion of a gene, e.g., an intron, a UTR, or a promotor region. In some embodiments, the gene encoding a lineage-specific cell-surface antigen (e.g., a sequence encoding an epitope bound by an agent that specifically binds the lineage-specific cell-surface antigen) comprises a regulatory region, e.g., an enhancer or inhibitor binding sequence. In some embodiments, the gene encoding a lineage-specific cell-surface antigen (e.g., a sequence encoding an epitope bound by an agent that specifically binds the lineage-specific cell-surface antigen) encodes a gene product (e.g., an mRNA and/or protein) characteristic of, or causally associated with, a disease or disorder. In some embodiments, the gene encoding a lineage-specific cell-surface antigen encodes a gene product (e.g., an mRNA and/or protein encoding an epitope bound by an agent that specifically binds the lineage-specific cell-surface antigen) that is not characteristic of, or causally associated with, a disease or disorder. In some embodiments, the gene encoding a lineage-specific cell-surface antigen comprises a sequence encoding a lineage-specific cell-surface antigen (e.g., a lineage-specific cell-surface antigen described herein). In some embodiments, the gene encoding a lineage-specific cell-surface antigen comprises an intronic sequence. In some embodiments, the gene encoding a lineage-specific cell-surface antigen comprises an expression regulatory sequence, e.g., a promoter or an enhancer. In some embodiments, the gene encoding a lineage-specific cell-surface antigen comprises a splice site.

In some embodiments, producing a genetic modification using HDR comprises contacting cells with a template polynucleotide, a CRISPR/Cas system, and one or more other agents (e.g., one or more HDR-promoting agents or expansion agents), e.g., contacting cells with a genetic modification mixture described herein. The disclosure provides, in part, methods and compositions that achieve unexpectedly high editing efficiencies utilizing HDR. In some embodiments, efficiency of HDR-mediated editing and/or efficiency of total/overall editing (HDR- and non-HDR-mediated) is determined by a method described herein (e.g., in Example 2). In some embodiments, the efficiency of HDR is at least 20, 30, 40, 50, 60, 70, 80, 90, 95, or 99% (e.g., 50%, 60%, 70%, 80%, 90% or higher). In some embodiments, contacting cells to produce a genetic modification using HDR comprises contacting cells with one or more HDR-promoting agents as described herein. Without wishing to be bound by theory, some aspects of this disclosure provide the discovery that the presence of one or more HDR-promoting agents may result in unexpectedly and advantageously high efficiency of HDR. Accordingly, methods describing contacting a cell herein also contemplate contacting a population of cells to produce a population of genetically modified cells, e.g., an editing efficiency, percent viability, and/or HDR efficiency described herein.

In some embodiments, producing a genetic modification using HDR comprises contacting a cell with a genetic modification mixture. As used herein, a genetic modification mixture refers to a mixture comprising a plurality of components used to genetically modify a gene encoding a lineage-specific cell-surface antigen (e.g., a sequence encoding an epitope bound by an agent that specifically binds the lineage-specific cell-surface antigen), e.g., in a cell. In some embodiments, a genetic modification mixture comprises one, two, three, or all of a CRISPR/Cas system, a template polynucleotide, one or more HDR-promoting agents, and one or more expansion agents. In some embodiments, a genetic modification mixture promotes HDR and HDR-mediated genetic modification (e.g., relative to another DNA repair pathway or genetic modifications utilizing another DNA repair pathway).

In some embodiments, contacting a cell with the genetic modification mixture comprises adding the genetic modification mixture directly to media comprising the cell. In some embodiments, contacting a cell with the genetic modification mixture comprises adding media comprising the genetic modification mixture to the cell or adding the cell to media comprising the genetic modification mixture. In some embodiments, the media is a growth media, e.g., a growth media suited to a hematopoietic cells (e.g., hematopoietic stem cells (HSCs)). Examples of growth media include, but are not limited to, a Stromal cell Growth Media (SCGM™, e.g. as available from Lonza Bioscience) or serum- and feeder-free media (SFFM). In some embodiments, contacting a cell with the genetic modification mixture comprises electroporating the genetic modification mixture or one or more components of the mixture into the cell. In some embodiments, contacting a cell with the genetic modification mixture comprises solvating the mixture in a lipid-permeable buffer, e.g., to serve as a carrier for movement of mixture components across the cell membrane. Examples of lipid-permeable buffers include, but are not limited to, DMSO and lipofectamine.

In some embodiments, the genetic modification mixture comprises a template polynucleotide, e.g., a single-strand donor oligonucleotide (ssODN), comprising a donor sequence, a first flanking sequence and a second flanking sequence. In some embodiments, the genetic modification mixture comprises a CRISPR/Cas system capable of producing a break, e.g., a double-stranded break, at a target site in the genome of the cell. In some embodiments, the genetic modification mixture comprises one or more other agents (e.g., an expansion agent and/or HDR-promoting agent) that promote genetic modification. In some embodiments, the template polynucleotide, e.g., ssODN, and the CRISPR/Cas system of the genetic modification mixture is mixed with the one or more other agents that promote genetic modification.

In some embodiments, HDR is induced by a DNA damage event that is capable of being mutagenic if left unrepaired or unprocessed, e.g., a double-stranded break. In some embodiments, the DNA damage event is induced by a CRISPR/Cas system, e.g., comprising a Cas nuclease, e.g., Cas9. Examples of DNA damage capable of producing a mutation include, but are not limited to, DNA alkylation, base deamination, base depurination, incidence of abasic sites, single-stranded breaks, and double-stranded breaks. Once DNA is damaged, the damage is repaired in multiple steps wherein cellular nucleases degrade nucleotide sequences at and proximal to the sites of the damage on one strand of the DNA. As used in this context, sequence “proximal” to the sites of damage is defined as a sequence that is found anywhere 10, 20, 30, 40, 50, 60, 70, 80, 90, 100, 150, 200, 250, 300, 350, 400, 450, or 500 nucleotides in the 5′ or 3′ direction of site of damage. Processing by nucleases, in turn, generates single-stranded overhangs comprised of a stretch of nucleotides that are not participating in base pairing interactions with nucleotides on the cognate strand to which the strand bearing the overhang is hybridized. Strand invasion follows, wherein the overhangs transiently base pair with a donor sequence that is located in close physical proximity to the damaged DNA molecule. In this way, template polynucleotide homology to a target site provided by the flanking sequences directs template polynucleotide participation in HDR. Strand invasion is followed by cellular polymerase-dependent recombination wherein the donor sequence serves as the template to direct the repair of the damaged DNA. Recombination between the donor sequence and the damaged DNA can incorporate the sequence of the donor sequence into the damaged DNA molecule. Following recombination, the repair is completed by a cellular ligase enzyme.

In some embodiments, a template polynucleotide comprises a first flanking sequence and a second flanking sequence, also referred to herein as a first homology sequence and a second homology sequence. In some embodiments, the first flanking sequence and second flanking sequence direct the binding of the template polynucleotide to a gene encoding a lineage-specific cell-surface antigen (e.g., a sequence encoding an epitope bound by an agent that specifically binds the lineage-specific cell-surface antigen) sequence in the cell. In some embodiments, a first flanking sequence is at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, or at least 250 nucleotides long (and optionally no more than 1000, no more than 750, no more than 500, no more than 400, no more than 300, or no more than 250 nucleotides long). In some embodiments, the first flanking sequence has at least 50%, at least 60%, at least 70%, at least at least 80%, at least 90%, at least 91%, at least 92%, at least 93%, at least 94%, at least 95%, at least 96%, at least 97%, at least 98%, at least 99%, or at least 100% identity to a sequence upstream of a DSB in the gene encoding a lineage-specific cell-surface antigen (e.g., upstream of a site where a DSB is produced by a CRISPR/Cas system described herein), or a sequence complementary thereto. In some embodiments, the first flanking sequence has 100% identity to a sequence upstream of a DSB in the gene encoding a lineage-specific cell-surface antigen (e.g., upstream of a site where a DSB is produced by a CRISPR/Cas system described herein), or a sequence complementary thereto. As used in this context, sequence “upstream” and “downstream” refer to a region within 10, within 20, within 30, within 40, within 50, within 60, within 70, within 80, within 90, or within 100 nucleotides of a feature in the DNA (e.g., a DSB), with each term referring to a different direction from the target site, and, in the case where the gene encoding a lineage-specific cell-surface antigen (e.g., a sequence encoding an epitope bound by an agent that specifically binds the lineage-specific cell-surface antigen) is a gene or portion thereof upstream is toward the transcription start site for the gene and downstream is away from the transcription start site for the gene. In some embodiments, the first flanking sequence is a 5′ homology arm of a template polynucleotide and is 5′ of a donor sequence, e.g., in an ssODN. In some embodiments, a second flanking sequence is at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, or at least 250 nucleotides in length (and optionally no more than 1000, no more than 750, no more than 500, no more than 400, no more than 300, or no more than 250 nucleotides in length). In some embodiments, the second flanking sequence has at least 50%, at least 60%, at least 70%, at least 80%, at least 90%, at least 91%, at least 92%, at least 93%, at least 94%, at least 95%, at least 96%, at least 97%, at least 98%, at least 99%, or at least 100% identity to a sequence downstream of a target site (e.g., downstream of a DSB produced by a CRISPR/Cas system in the target site), or a sequence complementary thereto. In some embodiments, the second flanking sequence has 100% identity to a sequence downstream of a DSB in the gene encoding a lineage-specific cell-surface antigen (e.g., downstream of a site where a DSB is produced by a CRISPR/Cas system described herein), or a sequence complementary thereto. In some embodiments, the second flanking sequence is a 3′ homology arm of a template polynucleotide and is 3′ of a donor sequence, e.g., in an ssODN. In some embodiments, the first flanking sequence and the second flanking sequence have identity or complementarity to different sequences within or proximal to the gene encoding a lineage-specific cell-surface antigen (e.g., a sequence encoding an epitope bound by an agent that specifically binds the lineage-specific cell-surface antigen). For example, in some embodiments the first flanking sequence has identity or complementarity to a first target sequence within or proximal to a gene encoding a lineage-specific cell-surface antigen (e.g., a sequence encoding an epitope bound by an agent that specifically binds the lineage-specific cell-surface antigen) and the second flanking sequence has identity or complementarity to a second target sequence within or proximal to the gene encoding a lineage-specific cell-surface antigen (e.g., a sequence encoding an epitope bound by an agent that specifically binds the lineage-specific cell-surface antigen). In some embodiments, the first target sequence and second target sequence are no more than 5, no more than 10, no more than 20, no more than 30, no more than 40, no more than 50, no more than 100, no more than 150, no more than 200, no more than 250, no more than 300, no more than 500, or no more than 1000 bases apart in the nucleic acid molecule comprising the gene encoding a lineage-specific cell-surface antigen. In some embodiments, the first flanking sequence has 100% identity to a sequence upstream of a DSB in the gene encoding a lineage-specific cell-surface antigen, or a sequence complementary thereto, and the second flanking sequence has 100% identity to a sequence downstream of a DSB in the gene encoding a lineage-specific cell-surface antigen, or a sequence complementary thereto.

In some embodiments, a flanking sequence (e.g., a 3′ homology arm or 5′ homology arm) comprises 2-100, 10-100, 20-100, 30-100, 40-100, 50-100, 60-100, 70-100, 80-100, 90-100, 2-150, 2-200, 2-250, 10-150, 10-200, 10-250, 50-150, 50-200, 50-250, 100-150, 100-200, 100-250, 150-200, 150-200, or 200-250 consecutive nucleotides that are 100% identical to a target sequence within a gene encoding a lineage-specific cell-surface antigen (e.g., a sequence encoding an epitope bound by an agent that specifically binds the lineage-specific cell-surface antigen). In some embodiments, a flanking sequence (e.g., a 3′ homology arm or 5′ homology arm) comprises at least 2, at least 10, at least 20, at least 30, at least 40, at least 50, at least 60, at least 70, at least 80, at least 90, or at least 100 consecutive nucleotides that are 100% identical to a target sequence within a gene encoding a lineage-specific cell-surface antigen (e.g., a sequence encoding an epitope bound by an agent that specifically binds the lineage-specific cell-surface antigen) (and optionally no more than 200, no more than 180, no more than 160, no more than 140, no more than 120, or no more than 100 consecutive nucleotides that are 100% identical to a target sequence within a gene encoding a lineage-specific cell-surface antigen. In some embodiments, a flanking sequence (e.g., a 3′ homology arm or a 5′ homology arm) comprises a nucleotide sequence that is 100% identical to a PAM sequence in the gene encoding a lineage-specific cell-surface antigen. In some embodiments, the nucleotide sequence identical to the PAM sequence is 2-3, 2-4, 2-5, 2-6, 3-4, 3-5, 3-6, 4-5, 4-6, or 5-6 nucleotides in length (e.g., 2, 3, 4, 5, or 6 nucleotides in length).

In some embodiments, a template polynucleotide comprises a donor sequence. In some embodiments, the donor sequence is integrated into a gene encoding a lineage-specific cell-surface antigen (e.g., a sequence encoding an epitope bound by an agent that specifically binds the lineage-specific cell-surface antigen) at the site of a DSB. In some embodiments, the donor sequence is homologous to the gene encoding a lineage-specific cell-surface antigen (e.g., a sequence encoding an epitope bound by an agent that specifically binds the lineage-specific cell-surface antigen) or a portion thereof, e.g., the sequence of the gene encoding a lineage-specific cell-surface antigen surrounding or adjacent to the DSB. In some embodiments, the donor sequence is contiguous with the first and second flanking sequences in a template polynucleotide. For example, in some embodiments a gene encoding a lineage-specific cell-surface antigen (such as a sequence encoding an epitope bound by an agent that specifically binds the lineage-specific cell-surface antigen) comprises a gene or a portion thereof, and the donor sequence is homologous to the gene encoding a lineage-specific cell-surface antigen (such as a sequence encoding an epitope bound by an agent that specifically binds the lineage-specific cell-surface antigen) or a portion thereof (e.g., in proximity to a DSB or a site targeted for a DSB by a CRISPR/Cas system as described herein). In some embodiments, the first and second flanking sequences guide binding of the template polynucleotide to a gene encoding a lineage-specific cell-surface antigen, facilitating interaction of the donor sequence with its homologous sequence in the gene encoding a lineage-specific cell-surface antigen and/or with cellular DNA repair (e.g., HDR) pathway components. In some embodiments, the donor sequence differs from a homologous sequence of the gene encoding a lineage-specific cell-surface antigen at 1, 2, 3, 4, 5, 6, 7, 8, 9, or 10 bases (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 bases), or at a number of positions corresponding to up to 1, 5, 10, 15, or 20% of the length of the donor sequence. In some embodiments, the donor sequence differs from a homologous sequence of the gene encoding a lineage-specific cell-surface antigen (e.g., a sequence encoding an epitope bound by an agent that specifically binds the lineage-specific cell-surface antigen) at no more than 1, no more than 2, no more than 3, no more than 4, no more than 5, no more than 6, no more than 7, no more than 8, no more than 9, or no more than 10 bases. In some embodiments, a donor sequence is 1-100, 1-80, 1-60, 1-40, 1-20, 1-15, 1-10, 1-9, 1-8, 1-7, 1-6, 1-5, 1-4, 1-3, 1-2, 5-100, 5-80, 5-60, 5-40, 5-20, 5-15, 5-10, 5-9, 5-8, 5-7, 5-6, 10-100, 10-80, 10-60, 10-40, 10-20, 10-15, 20-100, 20-80, 20-60, 20-40, 60-100, or 60-80 nucleotides in length (e.g., 1-10, 1-7, 1-5, or 1-3 nucleotides in length). In some embodiments, a donor sequence is no more than 100, no more than 90, no more than 80, no more than 70, no more than 60, no more than 50, no more than 45, no more than 40, no more than 35, no more than 30, no more than 25, no more than 20, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 base long. In some embodiments, a donor sequence is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 bases long. In some embodiments, a donor sequence differs from a homologous sequence of the gene encoding a lineage-specific cell-surface antigen at a position or positions corresponding to an epitope modification (e.g., a point mutation) in the gene encoding a lineage-specific cell-surface antigen (e.g., characteristic of, or causally associated with, a disease or disorder, or risk of developing a disease or disorder). In some embodiments, the donor sequence comprises sequence corresponding to the wild-type, functional, and/or naturally-occurring sequence at a position or positions corresponding to an epitope modification (e.g., a point mutation) in the gene encoding a lineage-specific cell-surface antigen (e.g., a sequence encoding an epitope bound by an agent that specifically binds the lineage-specific cell-surface antigen). In some embodiments, the donor sequence comprises an artificial or heterologous sequence.

A schematic of an exemplary template polynucleotide, an ssODN, is provided below:
[5′-homology arm]-[donor sequence]-[3′ homology arm]

Each homology arm (e.g., a flanking sequence described herein) has homology to a sequence in the gene encoding a lineage-specific cell-surface antigen proximal to the sequence homologous to the donor sequence.

In some embodiments, a homology arm comprises a sequence homologous to a PAM sequence in the gene encoding a lineage-specific cell-surface antigen (e.g., a sequence encoding an epitope bound by an agent that specifically binds the lineage-specific cell-surface antigen). In some embodiments, a CRISPR/Cas system for use in a method of the disclosure comprises a Cas nuclease that recognizes a PAM sequence in the gene encoding a lineage-specific cell-surface antigen and cuts the gene encoding a lineage-specific cell-surface antigen at a position near to the PAM sequence (e.g., 5′ or 3′ of the PAM sequence). Accordingly, in some embodiments a PAM homologous sequence is present in a 3′ homology arm or a 5′ homology arm of a template polynucleotide. In some embodiments, the PAM homologous sequence is positioned such that HDR of a DSB produced by a Cas nuclease promotes integration of a donor sequence. In some embodiments, the DSB is positioned in a gene encoding a lineage-specific cell-surface antigen (e.g., a sequence encoding an epitope bound by an agent that specifically binds the lineage-specific cell-surface antigen) sequence homologous to the donor sequence.

A schematic of an exemplary 3′ homology arm (e.g., where a CRISPR/Cas system (e.g., comprising Cas9) cuts a gene encoding a lineage-specific cell-surface antigen (e.g., a sequence encoding an epitope bound by an agent that specifically binds the lineage-specific cell-surface antigen) 5′ of a PAM sequence) is provided below:
[N]ₓ-[PAM]-[N]_{y}.

For example, an exemplary Cas nuclease, Cas9, cuts a gene encoding a lineage-specific cell-surface antigen (e.g., a sequence encoding an epitope bound by an agent that specifically binds the lineage-specific cell-surface antigen) 3-4 nucleotides 5′ of a PAM sequence. In some embodiments, x is 3-4, and y is the number of nucleotides in the remaining length of the homology arm (e.g., wherein the length of the homology arm is described herein). For example, for x=3, and a homology arm length of 100 nucleotides, y would be 100 minus 3 and minus the length of the PAM homologous sequence (e.g., where the PAM sequence is 3 nucleotides long, y would be 94 (100-3-3). In some embodiments, x is 2 and the homology arm is 50-60 nucleotides long. In some embodiments, x is 2 and the homology arm is 60-70 nucleotides long. In some embodiments, x is 2 and the homology arm is 70-80 nucleotides long. In some embodiments, x is 2 and the homology arm is 80-90 nucleotides long. In some embodiments, x is 2 and the homology arm is 90-100 nucleotides long. In some embodiments, x is 2 and the homology arm is 100-110 nucleotides long. In some embodiments, x is 2 and the homology arm is 110-120 nucleotides long. In some embodiments, x is 2 and the homology arm is 120-130 nucleotides long. In some embodiments, x is 2 and the homology arm is 130-140 nucleotides long. In some embodiments, x is 2 and the homology arm is 140-150 nucleotides long. In some embodiments, x is 2 and the homology arm is 150-160 nucleotides long. In some embodiments, x is 2 and the homology arm is 160-170 nucleotides long. In some embodiments, x is 2 and the homology arm is 170-180 nucleotides long. In some embodiments, x is 2 and the homology arm is 180-190 nucleotides long. In some embodiments, x is 2 and the homology arm is 190-200 nucleotides long. In some embodiments, x is 2 and the homology arm is 210-220 nucleotides long. In some embodiments, x is 2 and the homology arm is 220-230 nucleotides long. In some embodiments, x is 2 and the homology arm is 230-240 nucleotides long. In some embodiments, x is 2 and the homology arm is 240-250 nucleotides long. In some embodiments, x is 3 and the homology arm is 50-60 nucleotides long. In some embodiments, x is 3 and the homology arm is 60-70 nucleotides long. In some embodiments, x is 3 and the homology arm is 70-80 nucleotides long. In some embodiments, x is 3 and the homology arm is 80-90 nucleotides long. In some embodiments, x is 3 and the homology arm is 90-100 nucleotides long. In some embodiments, x is 3 and the homology arm is 100-110 nucleotides long. In some embodiments, x is 3 and the homology arm is 110-120 nucleotides long. In some embodiments, x is 3 and the homology arm is 120-130 nucleotides long. In some embodiments, x is 3 and the homology arm is 130-140 nucleotides long. In some embodiments, x is 3 and the homology arm is 140-150 nucleotides long. In some embodiments, x is 3 and the homology arm is 150-160 nucleotides long. In some embodiments, x is 3 and the homology arm is 160-170 nucleotides long. In some embodiments, x is 3 and the homology arm is 170-180 nucleotides long. In some embodiments, x is 3 and the homology arm is 180-190 nucleotides long. In some embodiments, x is 3 and the homology arm is 190-200 nucleotides long. In some embodiments, x is 3 and the homology arm is 210-220 nucleotides long. In some embodiments, x is 3 and the homology arm is 220-230 nucleotides long. In some embodiments, x is 3 and the homology arm is 230-240 nucleotides long. In some embodiments, x is 3 and the homology arm is 240-250 nucleotides long. In some embodiments, x is 4 and the homology arm is 50-60 nucleotides long. In some embodiments, x is 4 and the homology arm is 60-70 nucleotides long. In some embodiments, x is 4 and the homology arm is 70-80 nucleotides long. In some embodiments, x is 4 and the homology arm is 80-90 nucleotides long. In some embodiments, x is 4 and the homology arm is 90-100 nucleotides long. In some embodiments, x is 4 and the homology arm is 100-110 nucleotides long. In some embodiments, x is 4 and the homology arm is 110-120 nucleotides long. In some embodiments, x is 4 and the homology arm is 120-130 nucleotides long. In some embodiments, x is 4 and the homology arm is 130-140 nucleotides long. In some embodiments, x is 4 and the homology arm is 140-150 nucleotides long. In some embodiments, x is 4 and the homology arm is 150-160 nucleotides long. In some embodiments, x is 4 and the homology arm is 160-170 nucleotides long. In some embodiments, x is 4 and the homology arm is 170-180 nucleotides long. In some embodiments, x is 4 and the homology arm is 180-190 nucleotides long. In some embodiments, x is 4 and the homology arm is 190-200 nucleotides long. In some embodiments, x is 4 and the homology arm is 210-220 nucleotides long. In some embodiments, x is 4 and the homology arm is 220-230 nucleotides long. In some embodiments, x is 4 and the homology arm is 230-240 nucleotides long. In some embodiments, x is 4 and the homology arm is 240-250 nucleotides long.

A schematic of an exemplary 5′ homology arm (e.g., where a CRISPR/Cas system (e.g., comprising Cas12a) cuts a gene encoding a lineage-specific cell-surface antigen (e.g., a sequence encoding an epitope bound by an agent that specifically binds the lineage-specific cell-surface antigen) 3′ of a PAM sequence) is provided below:
[N]ₐ-[PAM]-[N]_{b}.

As a further example, another exemplary Cas nuclease, Cas12a, cuts a gene encoding a lineage-specific cell-surface antigen (e.g., a sequence encoding an epitope bound by an agent that specifically binds the lineage-specific cell-surface antigen) 18-19 nucleotides 3′ of a PAM sequence. In some embodiments, b is 18-19, and a is the number of nucleotides in the remaining length of the homology arm (e.g., wherein the length of the homology arm is described herein). For example, for b=18, and a homology arm length of 100 nucleotides, a would be 100 minus 18 and minus the length of the PAM homologous sequence (e.g., where the PAM sequence is 3 nucleotides long, a would be 79 (100-18-3). In some embodiments, b is 17 and the homology arm is 50-60 nucleotides long. In some embodiments, b is 17 and the homology arm is 60-70 nucleotides long. In some embodiments, b is 17 and the homology arm is 70-80 nucleotides long. In some embodiments, b is 17 and the homology arm is 80-90 nucleotides long. In some embodiments, b is 17 and the homology arm is 90-100 nucleotides long. In some embodiments, b is 17 and the homology arm is 100-110 nucleotides long. In some embodiments, b is 17 and the homology arm is 110-120 nucleotides long. In some embodiments, b is 17 and the homology arm is 120-130 nucleotides long. In some embodiments, b is 17 and the homology arm is 130-140 nucleotides long. In some embodiments, b is 17 and the homology arm is 140-150 nucleotides long. In some embodiments, b is 17 and the homology arm is 150-160 nucleotides long. In some embodiments, b is 17 and the homology arm is 160-170 nucleotides long. In some embodiments, b is 17 and the homology arm is 170-180 nucleotides long. In some embodiments, b is 17 and the homology arm is 180-190 nucleotides long. In some embodiments, b is 17 and the homology arm is 190-200 nucleotides long. In some embodiments, b is 17 and the homology arm is 210-220 nucleotides long. In some embodiments, b is 17 and the homology arm is 220-230 nucleotides long. In some embodiments, b is 17 and the homology arm is 230-240 nucleotides long. In some embodiments, b is 17 and the homology arm is 240-250 nucleotides long. In some embodiments, b is 18 and the homology arm is 50-60 nucleotides long. In some embodiments, b is 18 and the homology arm is 60-70 nucleotides long. In some embodiments, b is 18 and the homology arm is 70-80 nucleotides long. In some embodiments, b is 18 and the homology arm is 80-90 nucleotides long. In some embodiments, b is 18 and the homology arm is 90-100 nucleotides long. In some embodiments, b is 18 and the homology arm is 100-110 nucleotides long. In some embodiments, b is 18 and the homology arm is 110-120 nucleotides long. In some embodiments, b is 18 and the homology arm is 120-130 nucleotides long. In some embodiments, b is 18 and the homology arm is 130-140 nucleotides long. In some embodiments, b is 18 and the homology arm is 140-150 nucleotides long. In some embodiments, b is 18 and the homology arm is 150-160 nucleotides long. In some embodiments, b is 18 and the homology arm is 160-170 nucleotides long. In some embodiments, b is 18 and the homology arm is 170-180 nucleotides long. In some embodiments, b is 18 and the homology arm is 180-190 nucleotides long. In some embodiments, b is 18 and the homology arm is 190-200 nucleotides long. In some embodiments, b is 18 and the homology arm is 210-220 nucleotides long. In some embodiments, b is 18 and the homology arm is 220-230 nucleotides long. In some embodiments, b is 18 and the homology arm is 230-240 nucleotides long. In some embodiments, b is 18 and the homology arm is 240-250 nucleotides long. In some embodiments, b is 19 and the homology arm is 50-60 nucleotides long. In some embodiments, b is 19 and the homology arm is 60-70 nucleotides long. In some embodiments, b is 19 and the homology arm is 70-80 nucleotides long. In some embodiments, b is 19 and the homology arm is 80-90 nucleotides long. In some embodiments, b is 19 and the homology arm is 90-100 nucleotides long. In some embodiments, b is 19 and the homology arm is 100-110 nucleotides long. In some embodiments, b is 19 and the homology arm is 110-120 nucleotides long. In some embodiments, b is 19 and the homology arm is 120-130 nucleotides long. In some embodiments, b is 19 and the homology arm is 130-140 nucleotides long. In some embodiments, b is 19 and the homology arm is 140-150 nucleotides long. In some embodiments, b is 19 and the homology arm is 150-160 nucleotides long. In some embodiments, b is 19 and the homology arm is 160-170 nucleotides long. In some embodiments, b is 19 and the homology arm is 170-180 nucleotides long. In some embodiments, b is 19 and the homology arm is 180-190 nucleotides long. In some embodiments, b is 19 and the homology arm is 190-200 nucleotides long. In some embodiments, b is 19 and the homology arm is 210-220 nucleotides long. In some embodiments, b is 19 and the homology arm is 220-230 nucleotides long. In some embodiments, b is 19 and the homology arm is 230-240 nucleotides long. In some embodiments, b is 19 and the homology arm is 240-250 nucleotides long.

In some embodiments, the first and second flanking sequence of the template polynucleotide (e.g., ssODN) comprise sequences complementarity to a first and second portion of a gene encoding a lineage-specific cell-surface antigen. In some embodiments, the first and second portions of a gene encoding a lineage-specific cell-surface antigen comprise or are proximal to a sequence encoding an epitope bound by an agent that specifically binds the lineage-specific cell-surface antigen.

In some embodiments, the lineage-specific cell-surface antigen is CD123. In some embodiments, the first portion of the CD123 gene comprises a portion of exon 3 or a sequence proximal to exon 3 wherein “proximal is defined as a sequence that is found anywhere 10, 20, 30, 40, 50, 60, 70, 80, 90, or 100 nucleotides in the 5′ or 3′ direction of exon 3 of the CD123 gene. In some embodiments, the second portion of the CD123 gene comprises a portion of exon 3 or a sequence proximal to exon 3. In some embodiments, the first portion of the CD123 gene comprises a portion of exon 4 or a sequence proximal to exon 4 wherein “proximal” is defined as a sequence that is found anywhere 10, 20, 30, 40, 50, 60, 70, 80, 90, or 100 nucleotides in the 5′ or 3′ direction of exon 4 of the CD123 gene. In some embodiments, the second portion of the CD123 gene comprises a portion of exon 4 or a sequence proximal to exon 4. In some embodiments, the first flanking sequence of the ssODN comprises a flanking sequence set forth in any of SEQ ID NO: 93-99. In some embodiments, the second flanking sequence of the ssODN comprises a flanking sequence set forth in any of SEQ ID NOs: 93-99.

In some embodiments, the donor sequence of the template polynucleotide (e.g., ssODN) comprises a homologous sequence to the sequence encoding amino acids 51, 59, 61, 82, or 84 in a wildtype CD123 gene as set forth in the nucleotide sequence provided in SEQ ID NO: 13 or as set forth in the amino acid sequence provided in SEQ ID NO: 15, or the sequence of a corresponding amino acid position in a homologous CD123 gene. In some embodiments, the donor sequence of the template polynucleotide, e.g., ssODN, comprises a sequence homologous to 1, 2, 3, 4, or all of the codons encoding E51, S59, P61, T82, or R84 in the wildtype CD123 gene, or a corresponding position in a homologous CD123 gene, and encodes a different amino acid at said position(s). In some embodiments, the donor sequence of the template polynucleotide, e.g., ssODN, comprises a sequence homologous to the codon encoding E51 in the wildtype CD123 gene, or a corresponding position in a homologous CD123 gene, and encodes an amino acid other than glutamic acid at said position (e.g., lysine or glycine). In some embodiments, the donor sequence of the template polynucleotide, e.g., ssODN, comprises a sequence homologous to the codon encoding S59 in the wildtype CD123 gene, or a corresponding position in a homologous CD123 gene, and encodes an amino acid other than serine at said position (e.g., phenylalanine or cysteine). In some embodiments, the donor sequence of the template polynucleotide, e.g., ssODN, comprises a sequence homologous to the codon encoding P61 in the wildtype CD123 gene, or a corresponding position in a homologous CD123 gene, and encodes an amino acid other than proline at said position (e.g., leucine). In some embodiments, the donor sequence of the template polynucleotide, e.g., ssODN, comprises a sequence homologous to the codon encoding T82 in the wildtype CD123 gene, or a corresponding position in a homologous CD123 gene, and encodes an amino acid other than threonine at said position (e.g., alanine). In some embodiments, the donor sequence of the template polynucleotide, e.g., ssODN, comprises a sequence homologous to the codon encoding R84 in the wildtype CD123 gene, or a corresponding position in a homologous CD123 gene, and encodes an amino acid other than arginine at said position (e.g., glutamine or alanine). In some embodiments, the donor sequence of the template polynucleotide, e.g., ssODN, comprises a donor sequence set forth in any one of SEQ ID NOs: 93-99. For example, a template polynucleotide comprising the sequence of any one of SEQ ID NOs: 93-99 can be used, for example, to genetically engineer a cell (e.g., a hematopoietic cell) to express a variant lineage-specific cell-surface antigen that is not bound or bound to a reduced degree by an agent that specifically binds the lineage-specific cell-surface antigen.

In some embodiments, the lineage-specific cell-surface antigen is CD38. In some embodiments, the first portion of the CD38 gene comprises a portion of exon 7 or a sequence proximal to exon 7 wherein “proximal is defined as a sequence that is found anywhere 10, 20, 30, 40, 50, 60, 70, 80, 90, or 100 nucleotides in the 5′ or 3′ direction of exon 7 of the CD38 gene. In some embodiments, the second portion of the CD38 gene comprises a portion of exon 7 or a sequence proximal to exon 7.

In some embodiments, the donor sequence of the template polynucleotide (e.g., ssODN) comprises a homologous sequence to the sequence encoding amino acids 202, 237 270, 271, 271, 272, 273, or 274 in a wildtype CD38 gene as set forth in the nucleotide sequence provided in SEQ ID NO: 61 or as set forth in the amino acid sequence provided in SEQ ID NO: 63, or the sequence of a corresponding amino acid position in a homologous CD38 gene. In some embodiments, the donor sequence of the template polynucleotide, e.g., ssODN, comprises a sequence homologous to 1, 2, 3, 4, or all of the codons encoding D202, T237, N270, Q272, or S274 in the wildtype CD38 gene, or a corresponding position in a homologous CD38 gene, and encodes a different amino acid at said position(s). In some embodiments, the donor sequence of the template polynucleotide, e.g., ssODN, comprises a sequence homologous to the codon encoding D202 in the wildtype CD38 gene, or a corresponding position in a homologous CD38 gene, and encodes an amino acid other than aspartic acid at said position (e.g., glycine). In some embodiments, the donor sequence of the template polynucleotide, e.g., ssODN, comprises a sequence homologous to the codon encoding T237 in the wildtype CD38 gene, or a corresponding position in a homologous CD38 gene, and encodes an amino acid other than threonine at said position (e.g., alanine). In some embodiments, the donor sequence of the template polynucleotide, e.g., ssODN, comprises a sequence homologous to the codon encoding N270 in the wildtype CD38 gene, or a corresponding position in a homologous CD38 gene, and encodes an amino acid other than asparagine at said position (e.g., alanine). In some embodiments, the donor sequence of the template polynucleotide, e.g., ssODN, comprises a sequence homologous to the codon encoding Q272 in the wildtype CD38 gene, or a corresponding position in a homologous CD38 gene, and encodes an amino acid other than glutamine at said position (e.g., alanine, histidine or arginine). In some embodiments, the donor sequence of the template polynucleotide, e.g., ssODN, comprises a sequence homologous to the codon encoding S274 in the wildtype CD38 gene, or a corresponding position in a homologous CD38 gene, and encodes an amino acid other than serine at said position (e.g., phenylalanine). In some embodiments, the donor sequence of the template polynucleotide, e.g., ssODN, comprises a donor sequence set forth in any one of SEQ ID NOs: 93-99. For example, a template polynucleotide comprising the sequence of any one of SEQ ID NOs: 93-99 can be used, for example, to genetically engineer a cell (e.g., a hematopoietic cell) to express a variant lineage-specific cell-surface antigen that is not bound or bound to a reduced degree by an agent that specifically binds the lineage-specific cell-surface antigen.

TABLE 15
Exemplary ssODNs for HDR Modification of CD123

| | Alternate | |
|---|---|---|
| | ssODN | |
| ssODN Name | Name | Sequence (length in nucleotides) |
| g29-ssODN | ss29 | aataaaataaaaaaaaaaaacaatagagagtatgatttaccggcataga |
| (E51K-w/SM) | | atagtcggcgtctttGacGcactTAatatcggtcacgttcctattgagat |
| | | cccaggtcaactgctgagcctttgctttcatccttaggttcgtgattggt |
| | | gggtttggatctaaaacggtgacaggttggagttcgaagagatacgtaag |
| | | (SEQ ID NO: 93; 200 nucleotides) |
| g31-ssODN | ss31 | aaataaatacataaatacataaataaataaaaaaaaaaaaaaaacaat |
| (E51K-w/SM) | | agagagtatgatttaccggcatagaatagtcggcgtctttGacGcactTA |
| | | atatcggtcacatttctgttaaggtcccaggtcaactgctgagcctttgc |
| | | tttcatccttaggttcgtgattggtgggtttggatctaaaacggtgacag |
| | | (SEQ ID NO: 94; 200 nucleotides) |
| g31-ssODN | ss31 180 | ataaatacataaataaataaaataaaaataaaaaaacaatagagagtatg |
| (E51K-w/SM)180 | | atttaccggcatagaatagtcggcgtctttGacGcactTAatatcggtca |
| | | catttctgttaaggtcccaggtcaactgctgagcctttgctttcatcctt |
| | | aggttcgtgattggtgggtttggatctaaa (SEQ ID NO: 95; 180 |
| | | nucleotides) |
| g31-ssODN | ss31 150 | aataaaataaaaataaaaaaacaatagagagtatgatttaccggcataga |
| (E51K-w/SM)150 | | atagtcggcgtctttGacGcactTAatatcggtcacatttctgttaaggt |
| | | cccaggtcaactgctgagcctttgctttcatccttaggttcgtgattggt |
| | | (SEQ ID NO: 96; 150 nucleotides) |
| g31-ssODN | ss31 120 | aaaaaacaatagagagtatgatttaccggcatagaatagtcggcgtcttt |
| (E51K-w/SM)120 | | GacGcactTAatatcggtcacatttctgttaaggtcccaggtcaactgct |
| | | gagcctttgctttcatcctt (SEQ ID NO: 97; 120 |
| | | nucleotides) |
| g31-ssODN | ss31 100 | agagagtatgatttaccggcatagaatagtcggcgtctttGacGcactTA |
| (E51K-w/SM)100 | | atatcggtcacatttctgttaaggtcccaggtcaactgctgagcctttgc |
| | | (SEQ ID NO: 98; 100 nucleotides) |
| g31-ssODN | ss31 80 | atttaccggcatagaatagtcggcgtctttGacGcactTAatatcggtca |
| (E51K-w/SM)80 | | catttctgttaaggtcccaggtcaactgct (SEQ ID NO: 99; 80 |
| | | nucleotides) |

### Nucleic Acid Modification

In some embodiments, a template polynucleotide, e.g., ssODN, provided herein comprises one or more nucleotides that are chemically modified. Nucleic acids comprising one or more nucleotides that are chemically modified are also referred to herein as modified nucleic acids. Chemical modifications of nucleotides have previously been described, and suitable chemical modifications include any modifications that are beneficial for nucleotides function and do not measurably increase any undesired characteristics, e.g., off-target effects, of a given gRNA. Suitable chemical modifications include, for example, those that make a nucleic acid less susceptible to endo-or exonuclease catalytic activity, and include, without limitation, phosphorothioate backbone modifications, 2′-O-Me-modifications (e.g., at one or both of the 3′ and 5′ termini), 2′F-modifications, replacement of the ribose sugar with the bicyclic nucleotide-cEt, 3′thioPACE (MSP) modifications, or any combination thereof. Additional suitable nucleic acid modifications will be apparent to the skilled artisan based on this disclosure, and such suitable nucleic acid modifications include, without limitation, those described, e.g., Eckstein, Antisense Nucleic Acid Drug Dev. 2000 Apr. 10 (2): 117-21, Rusckowski et al. Antisense Nucleic Acid Drug Dev. 2000 Oct. 10 (5): 333-45, Stein, Antisense Nucleic Acid Drug Dev. 2001 Oct. 11 (5): 317-25, Vorobjev et al. Antisense Nucleic Acid Drug Dev. 2001 Apr. 11 (2): 77-85, Duffy. BMC Bio. 2020 Sep. 2 (8): 112, and U.S. Pat. No. 5,684,143, each of which is incorporated herein by reference in its entirety. In some embodiments, a template polynucleotide comprises a modified nucleotide positioned within the template polynucleotide as described herein with regard to guide RNAs (e.g., with regard to proximity to a 3′ or 5′ end of the template polynucleotide).

### Genetic Modification Mixtures

Some aspects of the present disclosure provide genetic modification mixtures. In some embodiments, producing a genetic modification using HDR comprises contacting cells (e.g., HSCs) with a genetic modification mixture comprising one or more other agents that promote genetic modification. In some embodiments, the one or more other agents comprise one or more expansion agents. In some embodiments, the one or more other agents comprise one or more HDR-promoting agents. In some embodiments, the one or more other agents comprise one or more expansion agents and one or more HDR-promoting agents. In some embodiments, producing a genetic modification using HDR comprises contacting HSCs with one or more HDR-promoting agents and/or one or more expansion agents.

As used herein, an “HDR-promoting agent” refers to a compound that increases the repair of DNA damage by the HDR pathway (e.g., relative to other DNA repair pathways and/or compared to otherwise similar conditions lacking the HDR-promoting agent). Examples of HDR-promoting agents include, but are not limited to: (a) SCR7 which is an inhibitor of DNA ligase IV that is responsible for the repair of DNA double-strand breaks via the non-homologous end joining repair pathway; (b) NU7441, which is an inhibitor of DNA-dependent protein kinase (DNA-PK), an enzyme involved in the non-homologous end joining DNA repair pathway; (c) Rucaparib, which is a poly ADP ribose polymerase (PARP) inhibitor that plays a role in the repair of single-stranded breaks in DNA through the base excision repair and nonhomologous end-joining pathways such that inhibition of PARP with rucaparib causes accumulation of single-strand breaks which ultimately results in double-stranded breaks enhancing homology-directed repair activity to promote genome integrity; and (d) RS-1, which is a stimulator of the human homologous recombination protein RAD51 that functions by stimulating binding of human RAD51 to single stranded DNA and enhances recombinogenic activity by stabilizing the active form of human RAD51 filaments without inhibiting human RAD51 ATPase activity.

In some embodiments, the genetic modification mixture comprises one or more HDR-promoting agents comprising SCR7. In some embodiments, the genetic modification mixture comprises one or more HDR-promoting agents comprising NU7441. In some embodiments, the genetic modification mixture comprises one or more HDR-promoting agents comprising rucaparib. In some embodiments, the genetic modification mixture comprises one or more HDR-promoting agents comprising RS-1. In some embodiments, contacting comprises culturing the cell (e.g., the HSCs) in media comprising the one or more HDR-promoting agents. In some embodiments, the cell is contacted with the one or more HDR-promoting agents prior to being contacted with a CRISPR/Cas system, e.g., Cas9, and/or prior to being contacted with a template polynucleotide. In some embodiments, a cell is contacted with a single HDR-promoting agent, e.g., a genetic modification mixture comprises a single HDR-promoting agent. In some embodiments, a cell is contacted with 2, 3, or 4 different HDR-promoting agent, e.g., the genetic modification mixture comprises 2, 3, or 4 different HDR-promoting agents. In some embodiments, a cell is contacted with the different HDR-promoting agents at the same time (e.g., by addition to culture media or by contact with a genetic modification mixture).

As used herein, an expansion agent refers to a compound that specifically promotes the proliferation, differentiation, and/or growth of CD34+ cells such as HSCs. In some embodiments, an expansion agent can be added to culture media. Examples of expansion agents include, but are not limited to: (a) human stem cell factor (hSCF), which is a protein that is critical for hematopoiesis and mast cell differentiation and also plays roles in survival and function of other cell types such as tumor and myeloid-derived suppressor cells wherein hSCF binding to receptor tyrosine kinases induces activation of AKT, ERK, JNK, and p38 pathways in target cells; (b) Fms-like tyrosine kinase 3 Ligand (FLT3-L), which is a hematopoietic cytokine that plays an important role as a co-stimulatory factor in the proliferation, differentiation, and survival of hematopoietic stem and progenitor cells and in the development of the immune system wherein FLT3-L exists as membrane-bound and soluble isoforms such that both isoforms are biologically active and signal through the class III tyrosine kinase receptor; (c) thrombopoietin (TPO), which is a key regulator of megakaryocytopoiesis and thrombopoiesis in vitro and in vivo, wherein TPO stimulates the proliferation and maturation of megakaryocytes and has an important role in regulating the level of circulating platelets in vivo; promoting the survival, self-renewal, and expansion of hematopoietic stem cells and primitive multilineage progenitor cells; (d) interleukin 6 (IL-6), which is a pleiotropic growth factor with a wide range of biological activities in immune regulation, hematopoiesis, and oncogenesis such that IL-6 is produced by a variety of cell types including T cells, B cells, monocytes and macrophages, fibroblasts, hepatocytes, vascular endothelial cells, and various tumor cell lines. IL-6 signals through a cell surface type I cytokine receptor complex consisting of the ligand-binding IL-6a (CD126) and the signal-transducing gp130 subunits and the binding of IL-6 to its receptor system induces activation of JAK/STAT signaling pathway; (e) StemRegenin (SR1), which is an antagonist of the aryl hydrocarbon receptor and promotes ex vivo expansion of CD34+ human hematopoietic stem cells and the generation of CD34+ hematopoietic progenitor cells from non-human primate induced pluripotent stem cells such that SR1 has been shown to collaborate with UM729 in preventing differentiation of acute myeloid leukemia (AML) cells in culture and stimulating the proliferation and differentiation of CD34+ hematopoietic progenitor cells into dendritic cells; and (f) UM171, which is a pyrimidoindole small molecule that was discovered in a screen of compounds capable of promoting CD34+ cell expansion when used in combination with other cytokines in culture.

In some embodiments, the genetic modification mixture comprises one or more expansion agents comprising hSCF. In some embodiments, the genetic modification mixture comprises one or more expansion agents comprising FLT3-L. In some embodiments, the genetic modification mixture comprises one or more expansion agents comprising TPO. In some embodiments, the genetic modification mixture comprises one or more expansion agents comprising IL-6. In some embodiments, the genetic modification mixture comprises one or more expansion agents comprising SR1. In some embodiments, the genetic modification mixture comprises one or more expansion agents comprising UM171. In some embodiments, contacting comprises culturing the cell (e.g., the HSCs) in media comprising the one or more expansion agents. In some embodiments, the cell is contacted with the one or more expansion agents prior to being contacted with CRISPR/Cas system, e.g., Cas9, and/or prior to being contacted with a template polynucleotide. In some embodiments, a cell is contacted with a single expansion agent, e.g., a genetic modification mixture comprises a single expansion agent. In some embodiments, a cell is contacted with 3, 4, or 5 different expansion agents, e.g., a genetic modification mixture comprises 2, 3, 4, or 5 different expansion agents. In some embodiments, a cell is contacted with the different expansion agents at the same time (e.g., by addition to culture media or by contact with a genetic modification mixture).

In some embodiments, a cell is contacted with 1, 2, 3, 4, or 5 expansion agents and 1, 2, 3, or 4 HDR-promoting agents, e.g., by addition to culture media or by contact with a genetic modification mixture comprising the aforementioned). In some embodiments, the cell is contacted with the one or more expansion agents and one or more HDR-promoting agents prior to being contacted with a CRISPR/Cas system, e.g., Cas9, and/or prior to being contacted with a template polynucleotide.

Other aspects of the present disclosure relate to kits for genetic modification of epitopes of lineage-specific cell-surface antigens. In some embodiments, producing a genetic modification using HDR comprises using a kit described herein. In some embodiments, producing a genetic modification using a base editor comprises using a kit described herein. In some embodiments, a kit comprises a collection of agents that, when used in combination with each other, produce a result such as genetic modification of HSCs. In some embodiments, a kit comprises instructions for use, e.g., instructions for producing a genetically modified HSC. In some embodiments, the instructions comprise instructions for a method described herein. In some embodiments, a kit, e.g., for genetic modification of HSCs, comprises: (a) a template polynucleotide (e.g., a single-strand donor oligonucleotide (ssODN) comprising a donor sequence, a first flanking sequence and a second flanking sequence); and (b) a CRISPR/Cas system capable of producing a double-stranded break at a target site in the genome of a cell, e.g., an HSC. In some embodiments, a kit comprises (c) one or both of: one or more expansion agents described herein, and one or more HDR promoting agent described herein. In some embodiments, a kit, e.g., for genetic modification of HSCs, comprises: (a) a gRNA; and (b) a base editor (or nucleic acid encoding a base editor) capable of introducing mutations at a target site in the genome of a cell, e.g., an HSC.

### Definitions

Antibody: As used herein, the term “antibody” refers to a polypeptide that includes canonical immunoglobulin sequence elements sufficient to confer specific binding to a particular target antigen. As is known in the art, intact antibodies as produced in nature are typically approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxy-terminal constant (CL) domain, separated from one another by another “switch”. Intact antibody tetramers comprise two heavy chain-light chain dimers in which the heavy and light chains are linked to one another by a single disulfide bond; two other disulfide bonds connect the heavy chain hinge regions to one another, so that the dimers are connected to one another and a tetramer is formed. Naturally-produced antibodies are also typically glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized in accordance with the present invention (e.g., as a component of a CAR) include glycosylated Fc domains, including Fc domains with modified or engineered glycosylation. In some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal. In some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody,” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as is used herein in the broadest sense and encompasses various antibody structures, including but not limited to monoclonal antibodies, polyclonal antibodies, multispecific antibodies (e.g., bispecific antibodies), and/or antibody fragments (preferably those fragments that exhibit the desired antigen-binding activity). An antibody described herein can be an immunoglobulin, heavy chain antibody, light chain antibody, LRR-based antibody, or other protein scaffold with antibody-like properties, as well as other immunological binding moiety known in the art, including, e.g., a Fab, Fab′, Fab′2, Fab2, Fab3, F (ab′) 2, Fd, Fv, Feb, scFv, SMIP, single domain antibody, single-chain antibody, diabody, triabody, tetrabody, minibody, maxibody, tandab, DVD, BiTe, TandAb, or the like, or any combination thereof. The subunit structures and three-dimensional configurations of different classes of antibodies are known in the art. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.).

Antigen-binding fragment: An “antigen-binding fragment” refers to a portion of an antibody that binds the antigen to which the antibody binds. An antigen-binding fragment of an antibody includes any naturally occurring, enzymatically obtainable, synthetic, or genetically engineered polypeptide or glycoprotein that specifically binds an antigen to form a complex. Exemplary antibody fragments include, but are not limited to, Fv, Fab, Fab′, Fab′-SH, F (ab′) 2; diabodies; single domain antibodies; linear antibodies; single-chain antibody molecules (e.g. scFv or VHH or VH or VL domains only); and multispecific antibodies formed from antibody fragments. In some embodiments, the antigen-binding fragments of the antibodies described herein are scFvs. In some embodiments, the antigen-binding fragments of the antibodies described herein are VHH domains only. As with full antibody molecules, antigen-binding fragments may be mono-specific or multispecific (e.g., bispecific). A multispecific antigen-binding fragment of an antibody may comprise at least two different variable domains, wherein each variable domain is capable of specifically binding to a separate antigen or to a different epitope of the same antigen.

Antibody heavy chain: As used herein, the term “antibody heavy chain” refers to the larger of the two types of polypeptide chains present in all antibody molecules in their naturally occurring conformations.

Antibody light chain: As used herein, the term “antibody light chain” refers to the smaller of the two types of polypeptide chains present in all antibody molecules in their naturally occurring conformations.

Synthetic antibody: As used herein, the term “synthetic antibody” refers to an antibody that is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Antigen: As used herein, the term “antigen” or “Ag” refers to a molecule that is capable of provoking an immune response. This immune response may involve either antibody production, the activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequences or a partial nucleotide sequence encoding a protein that elicits an immune response encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

Autologous: As used herein, the term “autologous” refers to any material derived from an individual to which it is later to be re-introduced into the same individual.

Allogeneic: As used herein, the term “allogeneic” refers to any material (e.g., a population of cells) derived from a different animal of the same species.

Hyperproliferative disease: As used herein, the term “hyperproliferative disease” refers to a disease characterized by the rapid and uncontrolled growth of aberrant cells. In some embodiments, a hyperproliferative disease is a benign or a malign disease. Malign diseases are typically characterized by the presence of malign cells, e.g., cancer cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

In certain embodiments, the hyperproliferative is a hematopoietic malignancy, such as a myeloid malignancy or a lymphoid malignancy. In some embodiments, the hematopoietic malignancy is Hodgkin's lymphoma, non-Hodgkin's lymphoma, leukemia, multiple myeloma, myelodysplastic syndrome, or blastic plasmacytoid dendritic cell neoplasm (BPDCN). In some embodiments, the hematopoietic malignancy is acute myeloid leukemia (AML), B-cell acute lymphoblastic leukemia (B-ALL), chronic myelogenous leukemia, acute lymphoblastic leukemia, or chronic lymphoblastic leukemia. In some embodiments, the hematopoietic malignancy is acute myeloid leukemia. In some embodiments, the hematopoietic malignancy is B-cell acute lymphoblastic leukemia. In some embodiments, the hematopoietic malignancy is myelodysplastic syndrome (MDS).

Conservative sequence modifications: As used herein, the term “conservative sequence modifications” refers to amino acid modifications that do not significantly affect or alter the binding characteristics of an antibody containing the amino acid sequence. Such conservative modifications include amino acid substitutions, additions and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Co-stimulatory ligand: As used herein, the term “co-stimulatory ligand” refers to a molecule on an antigen presenting cell (e.g., an APC, dendritic cell, B cell, and the like) that specifically binds a cognate co-stimulatory molecule on an immune cell (e.g., a T lymphocyte), providing a signal which mediates an immune cell response, including, but not limited to, proliferation, activation, differentiation, and the like. A co-stimulatory ligand can include, but is not limited to, CD7, B7-1 (CD80), B7-2 (CD86), CD28, PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on an immune cell (e.g., a T lymphocyte), such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

Cytotoxic: As used herein, the term “cytotoxic” or “cytotoxicity” refers to killing or damaging cells. In one embodiment, cytotoxicity of the metabolically enhanced cells is improved, e.g. increased cytolytic activity of immune cells (e.g., T lymphocytes).

Effective amount: As used herein, an “effective amount” as described herein refers to a dose that is adequate to prevent or treat a neoplastic disease, e.g., a cancer, in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, perhaps using the genetically engineered cells of the disclosure (e.g., CAR cells) in each or various rounds of administration, for example in temporal proximity with edited hematopoietic stem cells, as described herein.

For purposes of the invention, the amount or dose of a genetically engineered cell comprising a heterologous nucleic acid comprising a CAR construct described herein that is administered should be sufficient to effect a therapeutic or prophylactic response in the subject or animal over a reasonable time frame. For example, the dose should be sufficient to bind to antigen, or detect, treat, or prevent cancer in a period of from about 2 hours or longer, e.g., about 12 to about 24 or more hours, from the time of administration. In certain embodiments, the time period could be even longer. The dose will be determined by the efficacy of the particular genetically engineered cells of the disclosure (e.g., CAR cells) and the condition of the animal (e.g., human), as well as the body weight of the animal (e.g., human) to be treated.

Effector function: As used herein, “effector function” or “effector activity” refers to a specific activity carried out by an immune cell in response to stimulation of the immune cell. For example, an effector function of a T lymphocyte includes, recognizing an antigen and killing a cell that expresses the antigen.

Endogenous: As used herein “endogenous” refers to any material from or produced inside a particular organism, cell, tissue or system.

Exogenous: As used herein, the term “exogenous” refers to any material introduced from or produced outside a particular organism, cell, tissue or system.

Expand: As used herein, the term “expand” refers to increasing in number, as in an increase in the number of cells, for example, immune cells, e.g., T lymphocytes, B lymphocytes, NK cells, and/or hematopoietic cells. In one embodiment, immune cells, e.g., T lymphocytes, B lymphocytes, NK cells, and/or hematopoietic cells that are expanded ex vivo increase in number relative to the number originally present in a culture. In another embodiment, immune cells, e.g., T lymphocytes, B lymphocytes, NK cells, and/or hematopoietic cells that are expanded ex vivo increase in number relative to other cell types in a culture. In some embodiments, expansion may occur in vivo. The term “ex vivo,” as used herein, refers to cells that have been removed from a living organism, (e.g., a human) and propagated outside the organism (e.g., in a culture dish, test tube, or bioreactor).

Functional Portion: As used herein, the term “functional portion” when used in reference to a CAR refers to any part or fragment of the CAR constructs of the invention, which part or fragment retains the biological activity of the CAR construct of which it is a part (the parent CAR construct). Functional portions encompass, for example, those parts of a CAR construct that retain the ability to recognize target cells, or detect, treat, or prevent cancer, to a similar extent, the same extent, or to a higher extent, as the parent CAR construct. In reference to the parent CAR construct, the functional portion can comprise, for instance, about 10%, about 25%, about 30%, about 50%, about 68%, about 80%, about 90%, about 95%, or more, of the parent CAR.

The functional portion can comprise additional amino acids at the amino or carboxy terminus of the portion, or at both termini, which additional amino acids are not found in the amino acid sequence of the parent CAR construct. Desirably, the additional amino acids do not interfere with the biological function of the functional portion, e.g., recognize target cells, detect cancer, treat or prevent cancer, etc. More desirably, the additional amino acids enhance the biological activity as compared to the biological activity of the parent CAR construct.

Functional Variant: As used herein, the term “functional variant,” as used herein, refers to a CAR construct, polypeptide, or protein having substantial or significant sequence identity or similarity to a parent CAR construct, which functional variant retains the biological activity of the CAR of which it is a variant. Functional variants encompass, for example, those variants of the CAR construct described herein (the parent CAR construct) that retain the ability to recognize target cells to a similar extent, the same extent, or to a higher extent, as the parent CAR construct. In reference to the parent CAR construct, the functional variant can, for instance, be at least about 30%, about 50%, about 75%, about 80%, about 90%, about 91%, about 92%, about 93%, about 94%, about 95%, about 96%, about 97%, about 98%, about 99% or more identical in amino acid sequence to the parent CAR construct. A functional variant can, for example, comprise the amino acid sequence of the parent CAR with at least one conservative amino acid substitution. Alternatively or additionally, the functional variants can comprise the amino acid sequence of the parent CAR construct with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR construct.

gRNA: The terms “gRNA” and “guide RNA” are used interchangeably throughout and refer to a nucleic acid that promotes the specific targeting or homing of a gRNA/Cas9 molecule complex to a target nucleic acid. A gRNA can be unimolecular (having a single RNA molecule), sometimes referred to herein as sgRNAs, or modular (comprising more than one, and typically two, separate RNA molecules). A gRNA may bind to a target domain in the genome of a host cell. In some embodiments, the gRNA (e.g., the targeting domain thereof) is partially or completely complementary to the target domain. The gRNA may also comprise a “scaffold sequence,” (e.g., a tracrRNA sequence), that recruits a Cas9 molecule to a target domain bound to a gRNA sequence (e.g., by the targeting domain of the gRNA sequence). The scaffold sequence may comprise at least one stem loop structure and recruits an endonuclease. Exemplary scaffold sequences can be found, for example, in Jinek, et al. Science (2012) 337 (6096): 816-821, Ran, et al. Nature Protocols (2013) 8:2281-2308, International Publication No. WO2014/093694, and International Publication No. WO2013/176772, which are incorporated by reference herein in their entireties.

Guide RNAs may vary in sequence but retain substantially the same activity and specificity. Thus, for the gRNAs used as described herein, the gRNA sequence preferably has at least 50%, at least 60%, at least 70%, at least at least 80%, at least 90%, at least 91%, at least 92%, at least 93%, at least 94%, at least 95%, at least 96%, at least 97%, at least 98%, at least 99%, or at least 100% identity to the sequences of the gRNAs provided herein and retain substantially the same activity and specificity. Alternatively, for the gRNAs used as described herein, the gRNA sequence can vary by 10, 9, 8, 7, 6, 5, 4, 3, 2, or 1 nucleotide relative to the sequences of the gRNAs provided herein and retain substantially the same activity and specificity.

Heterologous: As used herein, the term “heterologous” refers to a phenomenon occurring in a living system, e.g., a cell, that does not naturally occur in that system. For example, expression of a protein in a cell, where the protein does not naturally occur in that cell (e.g., the cell does not naturally encode that protein), would be heterologous expression of the protein. In some embodiments, the heterologous nucleic acid encodes a chimeric antigen receptor construct.

Immune cell: As used herein, the term “immune cell,” used interchangeably herein with the term “immune effector cell,” refers to a cell that is involved in an immune response, e.g., promotion of an immune response. Examples of immune cells include, but are not limited to, T-lymphocytes, natural killer (NK) cells, macrophages, monocytes, dendritic cells, neutrophils, eosinophils, mast cells, platelets, large granular lymphocytes, Langerhans' cells, or B-lymphocytes. A source of immune cells (e.g., T lymphocytes, B lymphocytes, NK cells) can be obtained from a subject.

Immune response: As used herein the term “immune response” refers to a cellular and/or systemic response to an antigen that occurs when lymphocytes identify antigenic molecules as foreign and induce the formation of antibodies and/or activate lymphocytes to remove the antigen.

Immunotherapeutic agent: As used herein the term “immunotherapeutic agent” refers to an agent that targets (e.g., specifically binds to) a lineage-specific cell-surface antigen, e.g., CLL-1, CD30, CD6, CD7, BCMA, CD123, CD38, CD5, CD47, CD34, EMR2, or CD19. Examples of immunotherapeutic agents include antibodies that target a lineage-specific cell-surface antigen, including multispecific antibodies (e.g., bispecific T cell engagers); antibody-drug conjugates (ADCs) comprising an antibody that targets a lineage-specific cell-surface antigen linked to a cytotoxic molecule; chimeric antigen receptors (CARs) that target a lineage-specific cell-surface antigen; and cells (such as immune effector cells, e.g. T cells or NK cells) comprising a chimeric antigen receptor that targets a lineage-specific cell-surface antigen (CAR T cells).

Mutation: As used herein, the term “mutation” refers to a change (e.g., an insertion, deletion, inversion, or substitution) in a nucleic acid sequence as compared to a reference sequence, e.g., the corresponding sequence of a cell not having such a mutation, or the corresponding wild-type nucleic acid sequence. In some embodiments provided herein, a mutation in a gene encoding lineage-specific cell-surface antigen results in expression of a variant form of the lineage-specific cell-surface antigen that is not bound by an immunotherapeutic agent targeting the lineage-specific cell-surface antigen, or bound at a significantly lower level than the non-mutated lineage-specific cell-surface antigen encoded by the gene. In some embodiments, a cell harboring a genomic mutation gene encoding a lineage-specific cell-surface antigen as provided herein is not bound by, or is bound at a significantly lower level by an immunotherapeutic agent that targets the lineage-specific cell-surface antigen, e.g., an anti-CD123 antibody or a CD123-targeted chimeric antigen receptor (CAR).

Nucleic acid: As used herein, the term “nucleic acid” refers to a polymer of at least three nucleotides. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid.” In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid is prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Single chain antibodies: As used herein, the term “single chain antibodies” refers to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird. Science (1988) 242:423-442; Huston et al. Proc. Natl. Acad. Sci. USA (1988) 85:5879-5883; Ward et al. Nature (1989) 334:54454; Skerra et al. Science (1988) 242:1038-1041.

Specifically binds: As used herein, the term “specifically binds,” with respect to an antigen binding domain, such as an antibody agent or a portion of a chimeric antigen receptor, refers to an antigen binding domain or antibody agent which recognizes a specific antigen, but does not substantially recognize or bind other molecules in a sample. For example, an antigen binding domain or antibody agent that specifically binds to an antigen from one species may also bind to that antigen from one or more species. But, such cross-species reactivity does not itself alter the classification of an antigen binding domain or antibody agent as specific. In another example, an antigen binding domain or antibody agent that specifically binds to an antigen may also bind to different allelic forms of the antigen. However, such cross reactivity does not itself alter the classification of an antigen-binding domain or antibody agent as specific. In some instances, the terms “specific binding” or “specifically binding,” can be used in reference to the interaction of an antigen binding domain or antibody agent, a protein, or a peptide with a second chemical species, to mean that the interaction is dependent upon the presence of a particular structure (e.g., an antigenic determinant or epitope) on the chemical species; for example, an antigen binding domain or antibody agent recognizes and binds to a specific protein structure rather than to proteins generally. If an antigen binding domain or antibody agent is specific for epitope “A”, the presence of a molecule containing epitope A (or free, unlabeled A), in a reaction containing labeled “A” and the antigen binding domain or antibody agent, will reduce the amount of labeled A bound to the antibody.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments, a human subject is an adult, adolescent, or pediatric subject. In some embodiments, a subject is suffering from a disease, disorder or condition, e.g., a disease, disorder, or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Target: As used herein, the term “target” refers to a cell, tissue, organ, or site within the body that is the subject of provided methods, systems, and/or compositions, for example, a cell, tissue, organ or site within a body that is in need of treatment or is preferentially bound by, for example, a CAR, as described herein.

Therapeutic: As used herein, the term “therapeutic” refers to a treatment and/or prophylaxis. A therapeutic effect is obtained by suppression, remission, or eradication of a disease state.

Transfected: As used herein, the term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Transgene: As used herein, the term “transgene” refers to an exogenous nucleic acid sequence comprised in a cell, e.g., in the genome of the cell, in which the nucleic acid sequence does not naturally occur. In some embodiments, a transgene may comprise or consist of a nucleic acid sequence encoding a gene product, e.g., a CAR. In some embodiments, a transgene may comprise or consist of an expression construct, e.g., a nucleic acid sequence encoding a gene product under the control of a regulatory element, e.g., a promoter.

Treat: As used herein, the term “treat,” “treatment,” or “treating” refers to partial or complete alleviation, amelioration, delay of onset of, inhibition, prevention, relief, and/or reduction in incidence and/or severity of one or more symptoms or features of a disease, disorder, and/or condition. In some embodiments, treatment is administered to a subject who does not exhibit signs or features of a disease, disorder, and/or condition (e.g., prophylactic). In some embodiments, treatment is administered to a subject who exhibits only early or mild signs or features of the disease, disorder, and/or condition, for example for the purpose of decreasing the risk of developing pathology associated with the disease, disorder, and/or condition. In some embodiments, treatment is administered to a subject who exhibits established, severe, and/or late-stage signs of the disease, disorder, or condition. In some embodiments, treating comprises administering to a subject an immune cell comprising a genetically engineered cell expressing a CAR (e.g., a T lymphocyte, B-lymphocyte, NK cell) or administering to a subject a hematopoietic stem cell transplant comprising genetically engineered stem cells.

Tumor: As used herein, the term “tumor” refers to an abnormal growth of cells or tissue. In some embodiments, a tumor comprises cells that are precancerous (e.g., benign), malignant, pre-metastatic, metastatic, and/or non-metastatic. In some embodiments, a tumor is associated with, or is a manifestation of, a cancer. In some embodiments, a tumor is a disperse tumor or a liquid tumor. In some embodiments, a tumor is a solid tumor.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D.N. Glover ed. 1985); Nucleic Acid Hybridization (B. D. Hames & S. J. Higgins eds. (1985); Transcription and Translation (B. D. Hames & S. J. Higgins, eds. (1984); Immobilized Cells and Enzymes (IRL Press, (1986); and B. Perbal, A practical Guide To Molecular Cloning (1984).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Epitope Modification of CD123

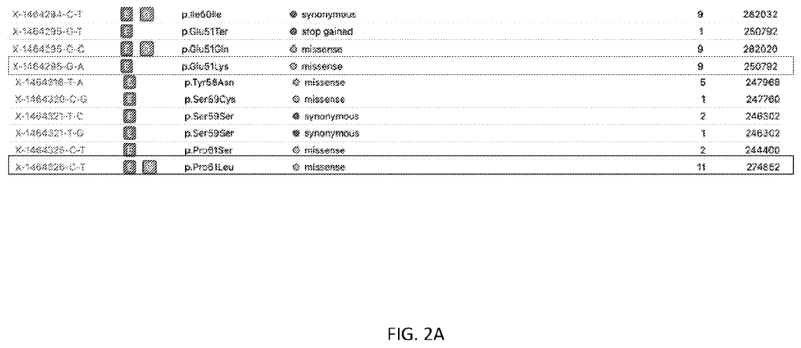

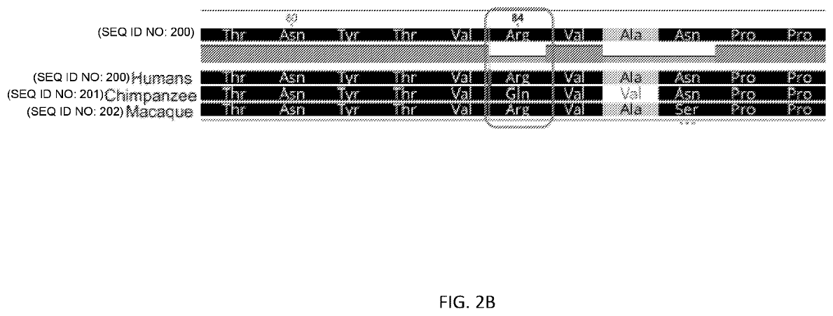

In some embodiments, the present disclosure provides methods for cell-specific targeting of therapeutic agents (e.g., antibodies) that recognize antigens (e.g. CD123) present on the cell surface. Analysis of a CD123 crystal structure identified binding sites important for CD123 antibody clone 7G3 (BD Biosciences catalog #561058) binding (FIGS. 1A and 1B). Five amino acids were identified for being important for 7G3 recognition of CD123 including E51, S59, P61, T82, and R84 (FIG. 1C). Anti-CD123 mouse antibody clone 7G3 has been humanized for use in humans and commercialization (FIG. 1D). Residues important for CD123 binding were searched in gnomAD which showed that many of the identified residues contain benign missense mutations in viable human subjects (FIG. 2A) and show conservation amongst non-human primates (FIG. 2B).

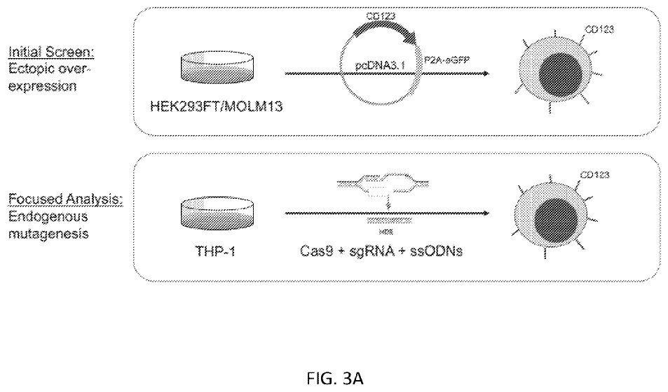

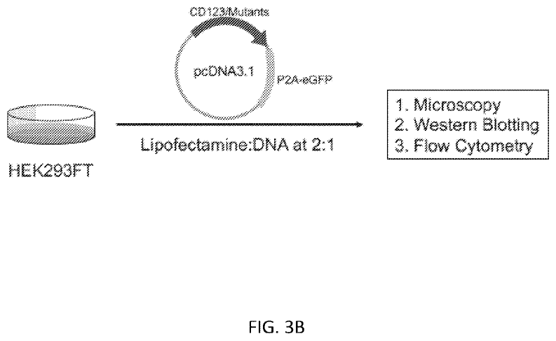

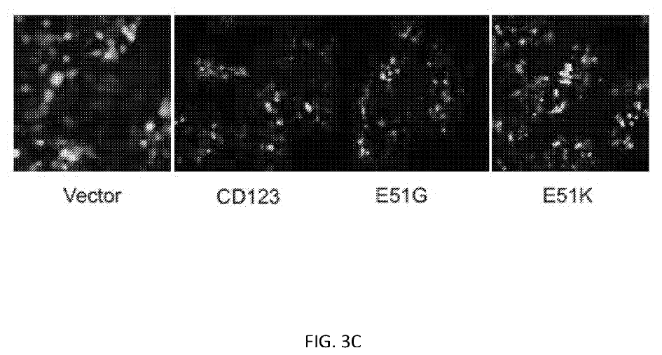

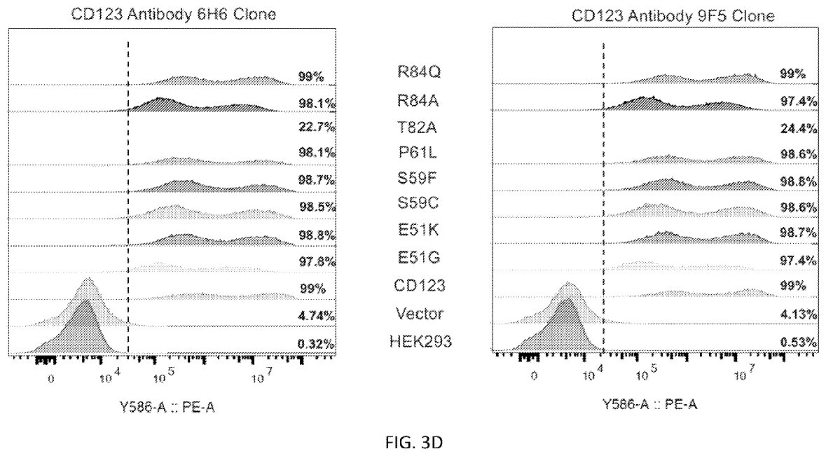

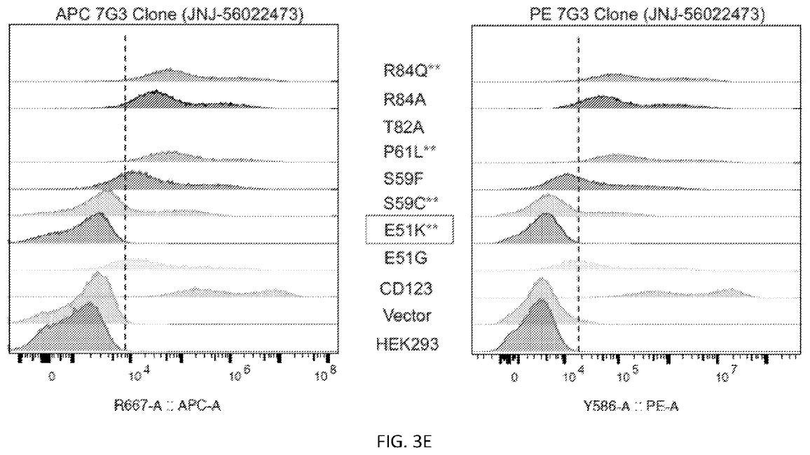

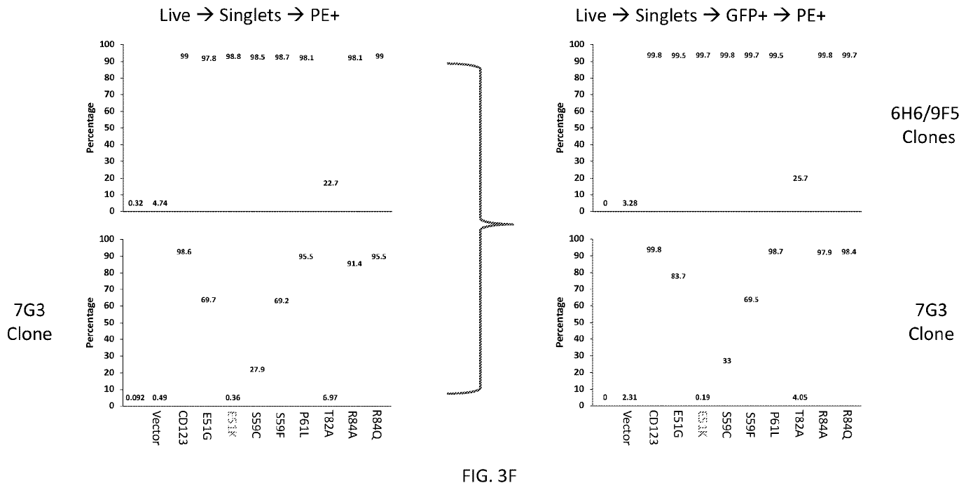

CD123 mutants were evaluated to determine if the mutations occurring at the 7G3 binding sites abolished antibody binding. Cell lines expressing CD123 comprising mutations at the 7G3 binding site were generated and screened using conventional methods well known in the art (FIGS. 3A-3C). After ectopic expression of CD123 and screening analyses, flow cytometry using two other antibodies that bind to CD123 (6H6, BioLegend catalog #306006; 9F5, BD Biosciences catalog #555644) was employed as positive controls for CD123 detection as these two antibody clones do not recognize amino acid residues E51, S59, P61, T82, or R84. Cells that lack CD123, such as HEK293 cells and cells expressing the vector backbone alone, were not detected when stained with 6H6 or 9F5. Flow cytometry analysis of cells expressing CD123 and CD123 mutants (R84Q, R84A, P61L, S59F, S59C, E51K, or E51G) showed positive staining with both 6H6 and 9F5 antibodies. The T82A CD123 mutant was not detectible by flow cytometry indicating that this mutant is not expressed on the cell surface (FIGS. 3D and 3F).

HEK293 cells and cells expressing vector alone showed no staining when contacted with 7G3. Cells expressing wild-type CD123 showed positive staining when contacted with 7G3. CD123 mutants showed varying levels of staining when contacted with 7G3 (FIG. 3E). The greatest reduction in staining in cells contacted with 7G3 relative to cells contacted with 6H6 or 9F5 was seen in the E51K mutant (FIG. 3F).

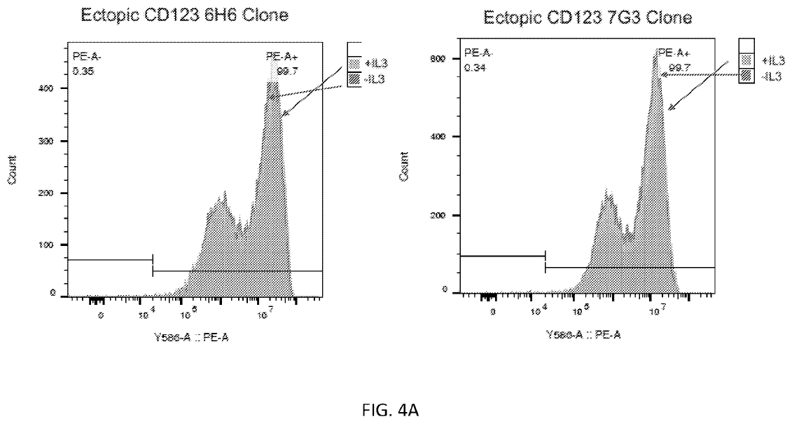

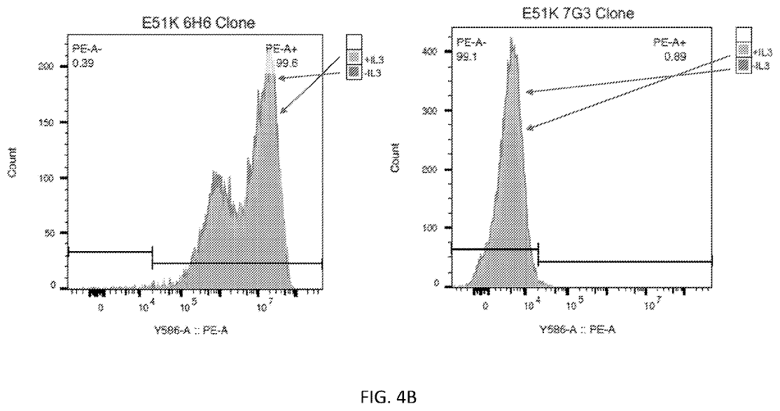

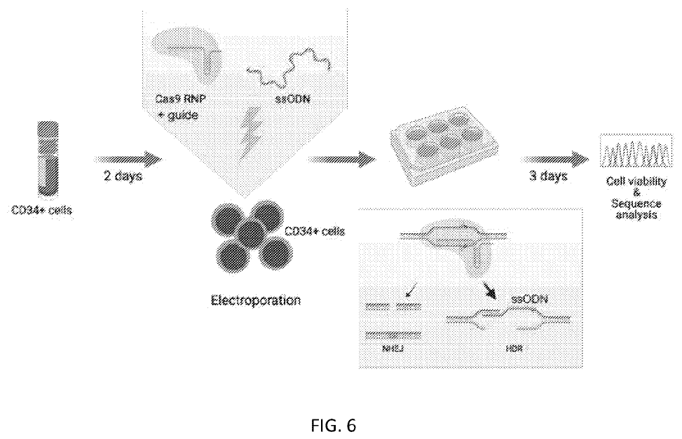

Flow cytometry was used to determine that IL3 does not inhibit binding of 6H6 or 7G3 to wild-type CD123 (FIG. 4A) or 6H6 or 7G3 binding to CD123 E51K (FIG. 4B). These results indicate that select CD123 epitopes, such as E51, can be mutated for selective targeting of antibody therapies to HSCs.

### Example 2: Epitope Modification of CD38

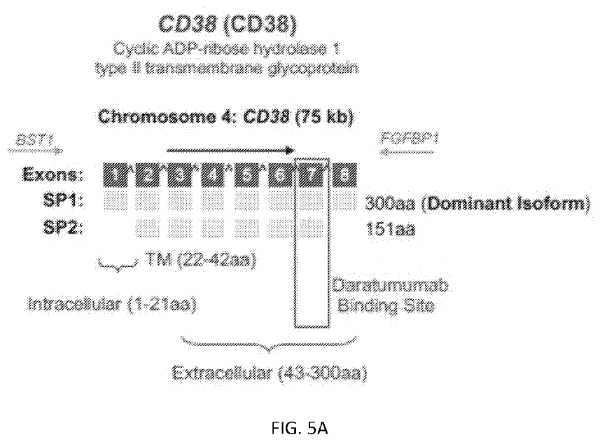

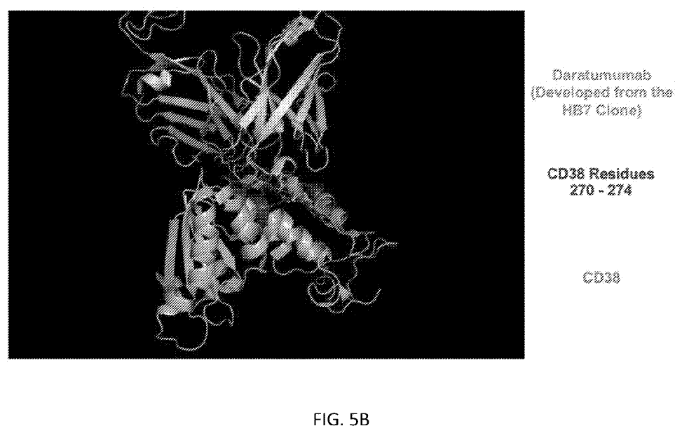

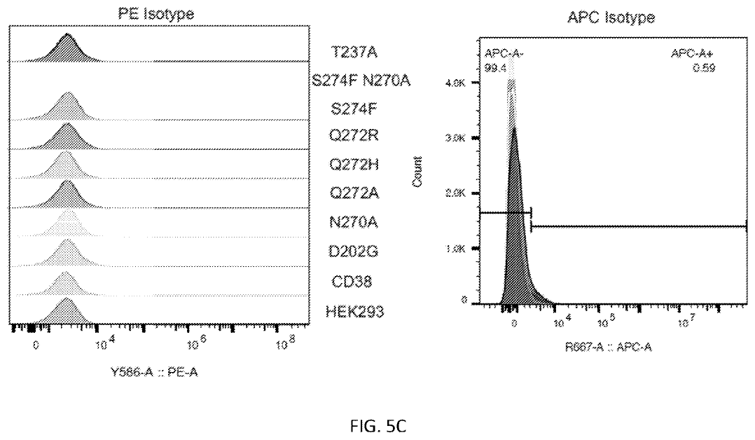

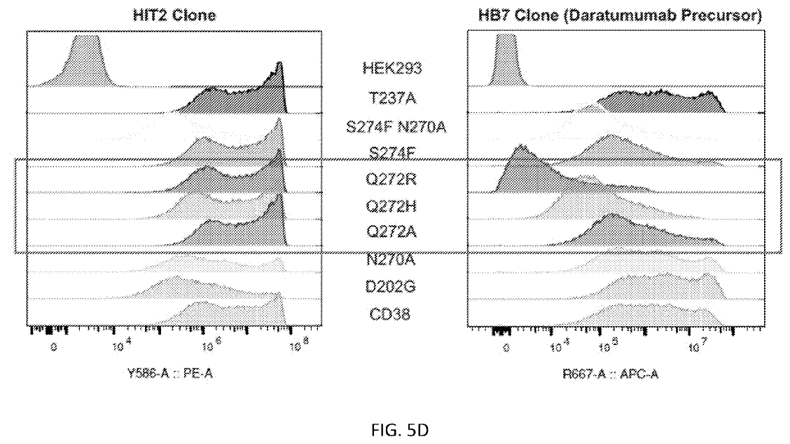

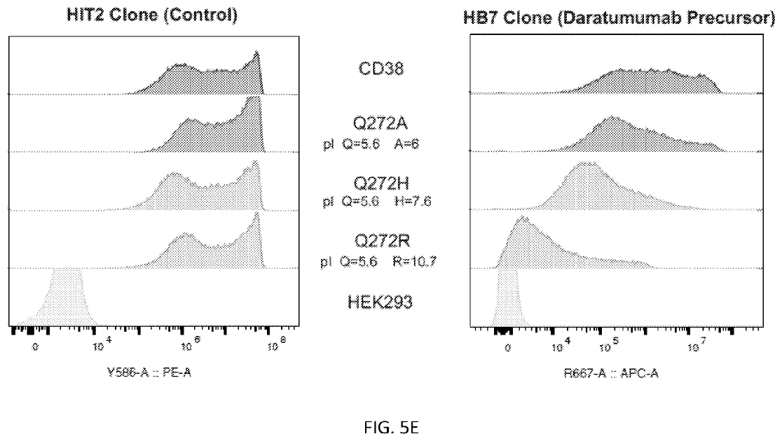

This example describes epitope modification of CD38 for selective targeting of CD38+ cells with antibodies. Daratumumab is an antibody that binds amino acids present in exon 7 of CD38 (residues 270-274) comprising an extracellular domain (FIG. 5A) as confirmed by crystal structure analysis (FIG. 5B).

HEK293T cells expressing CD38 mutants comprising mutations at the daratumumab binding site were constructed using conventional methods well known in the art. When cells were contacted with IgG1k isotype control labeled with fluorophore (either allophycocyanin (APC) or phycoerythrin (PE)), there was no difference in staining between CD38-deficient HEK293 cells, cells expressing wild-type CD38, or cells expressing CD38 mutants (FIG. 5C). Contacting HEK293 cells with a control antibody (HIT2) that binds to CD38 but does not recognize residues 270-274 showed no staining (FIGS. 5D and 5E). However, contacting cells expressing either wild-type or mutant versions of CD38 with HIT2 showed positive staining (FIGS. 5D and 5E). Contacting cells with HB7 (an antibody that binds CD38 in the same region as daratumumab) showed no staining in HEK293 cells and positive staining in cells expressing wild-type CD38 (FIGS. 5D and 5E). Flow cytometry analysis of CD38 mutants indicated that mutation of glutamine 272 in exon 7 of CD38 reduced or abolished binding of HB7 (FIGS. 5D and 5E). This data indicates that epitope modification of CD38 can be employed for selective targeting of antibody therapies to HSCs.

### Example 3: Epitope Modification of CD123 Via HDR-Editing

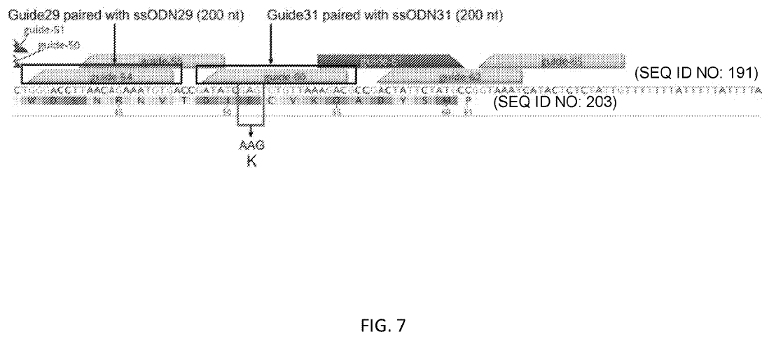

This example describes an exemplary method for HDR-editing of HSCs for epitope modification of CD123. In some embodiments of the present disclosure, methods are provided for editing of HSCs using CRISPR and HDR pathways by employing a ssODN as a template for genomic repair. In some embodiments, CD34+ HSCs are electroporated with guide RNAs (gRNAs; alternatively referred to as “g” followed by a number, such as g29 and g31), Cas9, and ssODNs (alternatively referred to as “ss” followed by a number, such as ss29 and ss31). Cells are then evaluated for gene editing outcomes by sequencing (FIG. 6). Two gRNAs (g29 (Guide 29) and g31 (Guide 31)) targeting select regions of CD123 were designed along with two ssODNs (ss29 and ss31) in order to induce an E51K mutation (FIG. 7). Sequencing analysis of HDR-edited cells showed that design of ssODNs to encode mutations closer to the cut site resulted in higher HDR efficiency.

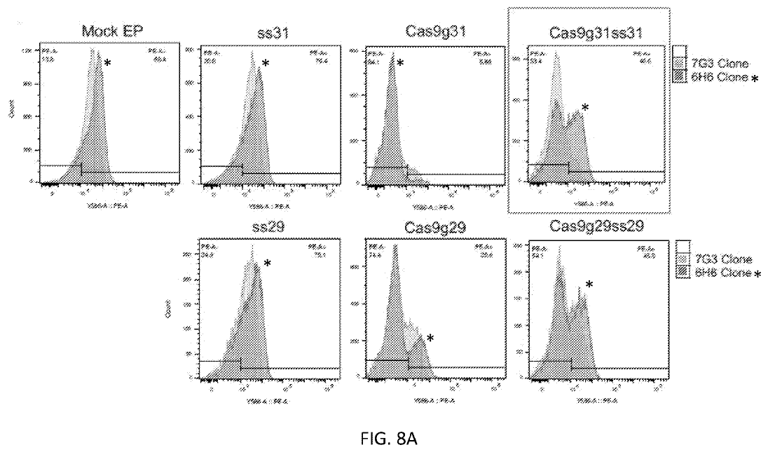

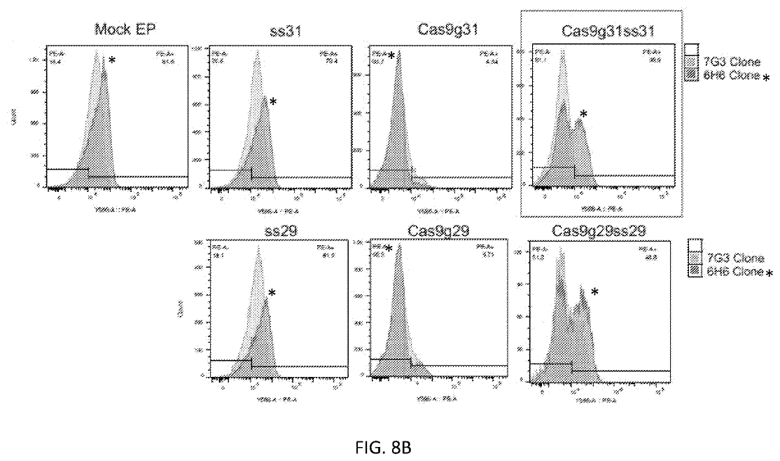

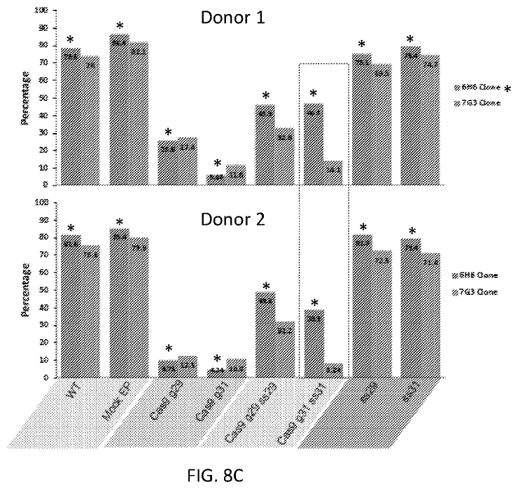

CD34+ cells from two donor HSC lines derived from healthy subjects were used for HDR editing. The donor cells were electroporated with Cas9, gRNAs, and ssODN as described above to generate a CD123 epitope modification comprising a E51K mutation. Flow cytometry analysis of wild-type cells, mock electroporated cells, and cells electroporated with ssODN alone (ss29 or ss31) showed similar levels of staining from both 6H6 and 7G3 after both donor lines were contacted with antibodies. Electroporation with Cas9 and gRNAs (g29 or g31) together resulted in a decrease in staining from both 6H6 and 7G3 in both donor cell populations after being contacted with antibodies. This result indicated that the presence of an ssODN is needed to prevent deletions in the edited locus following electroporation with Cas9 and gRNAs. When donor cell populations were electroporated with Cas9, gRNA, and ssODN, flow cytometry analysis showed 7G3 staining was decreased relative to 6H6 staining after contacting edited cells with these antibodies (FIGS. 8A and 8B). Electroporation with the combination of g31 and ss31 resulted in greater reductions in 7G3 staining relative to 6H6 staining when compared to cells that were electroporated with the combination of g29 and ss29 (FIGS. 8A-8C). These results indicate that epitope modification was achieved via HDR-editing in the donor cells.

Genome editing outcomes were characterized in HSCs to determine the effect of electroporation conditions on mutation incidence. Two separate CD34+ donor cells (Donor land Donor 2) and a CD123+ cell line “TIB-202 (THP-1)”) were untreated, mock electroporated, or electroporated with Cas9+ ssODNs, Cas9+ gRNAs, or with Cas9+ssODN+ gRNAs (FIG. 9A; note that “g54”=g29 and “g60”=g31 as used above, see FIG. 7). DNA was then harvested and used to prepare a next generation sequencing (NGS) library and subjected to NGS analysis (FIG. 9A). DNA sequencing controls were verified using RAMP-Seq (FIG. 9B).

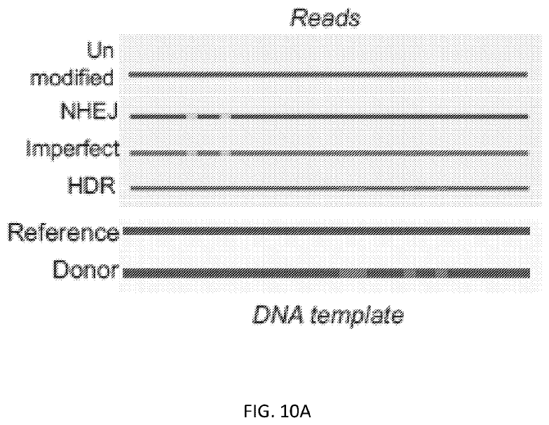

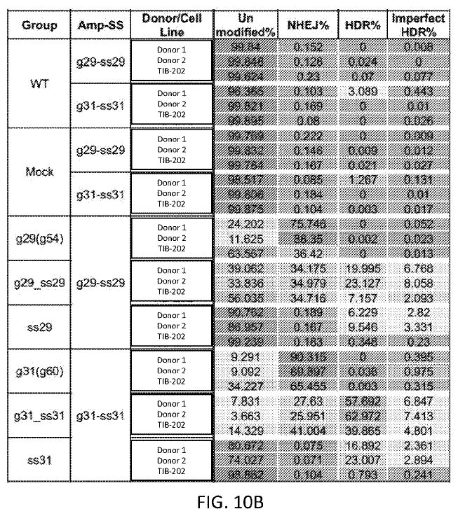

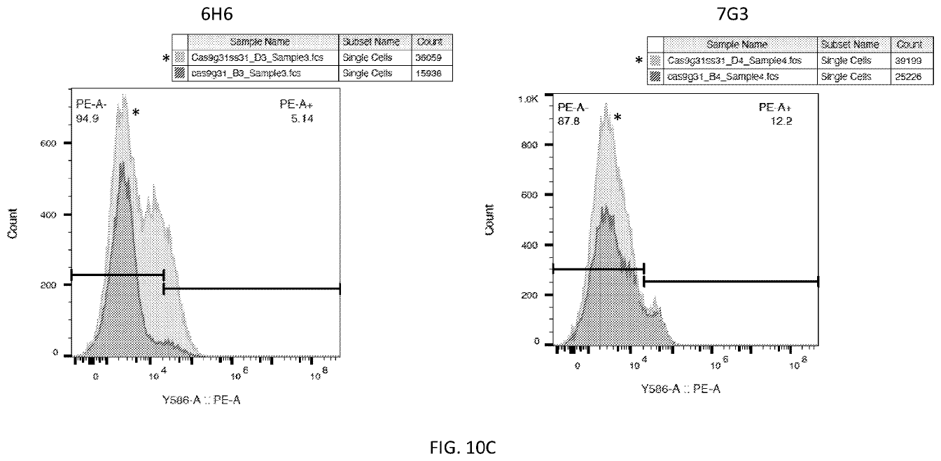

As shown in FIG. 10A, it was predicted that four outcomes were possible based on electroporation conditions: (1) no mutational change in the genome (“unmodified”); (2) non-homologous end-joining due to failure to use a repair template (e.g., an ssODN) for repair of Cas9-dependent genomic cleavage (“NHEJ”); (3) imperfect HDR of the cleaved genomic locus comprising a combination of HDR and NHEJ events (“imperfect”); and (4) HDR-dependent repair incorporating the mutation encoded by the ssODN (“HDR”). When both donors and TIB-202 cells were untreated or mock electroporated, NGS analysis showed that genomic loci were almost entirely unmodified in all three cell populations. When donor 1 and donor 2 cells were electroporated with ssODN and Cas9, NGS analysis (FIG. 10B) showed a slight increase in HDR and imperfect HDR editing events in the donor 1 and donor 2 cell populations but no significant increase in the TIB-202 cell population. When donor cells were electroporated with gRNAs and Cas9, NGS analysis (FIG. 10B) showed that NHEJ events comprised the vast majority of genomic editing outcomes in all three cell populations (both donor and TIB-202 cells) with HDR and imperfect HDR events not exceeding background levels. When donor or TIB-202 cells were electroporated with the combination of Cas9, ssODN, and gRNAs, NGS analysis (FIG. 10B) showed a significant increase in HDR events and a significant decrease in NHEJ events in both donor cells and TIB-202 cell populations. Electroporation with the combination of Cas9, ssODN, and gRNAs resulted in a negligible increase in imperfect HDR events relative to donor cell populations electroporated with Cas9 and ssODN (FIG. 10B).

Flow cytometry was employed for analysis of donor 2 cells that were electroporated with Cas9+g31 or Cas9+ss31+g31 to verify the epitope modifications on CD123 proteins expressed in donor cells. Flow cytometry analysis (FIG. 10C) showed that electroporation with Cas9+g31 resulted in significantly decreased 6H6 staining relative to cells electroporated with Cas9+ss31+g31, indicating substantial deletions in the CD123 amino acid sequence and/or reduced CD123 localized to the cell surface when electroporated with Cas9+g31 but not ss31. Flow cytometry analysis of electroporated donor cells stained with 7G3 showed no significant difference in 7G3 staining between the two edited populations indicating that both Cas9+g31 and Cas9+ss31+g31 abolished epitopes required for 7G3 antibody binding (FIG. 10C).

### Example 4: Base Editing for Epitope Modification on Lineage-Specific Cell-Surface Antigens

This example describes base editing as a genetic engineering approach to generate cells comprising variant forms of lineage-specific cell-surface antigens (e.g., CLL-1, CD30, CD6, CD7, BCMA, CD123, CD38, CD47, CD5, CD34, EMR2, or CD19).

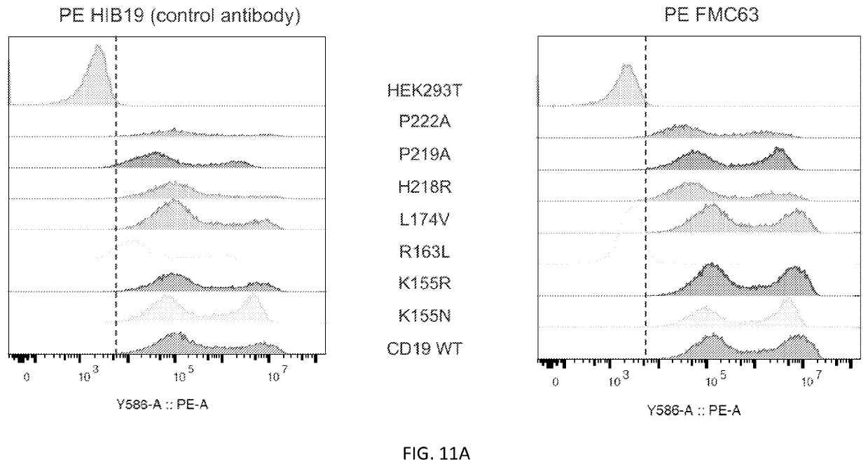

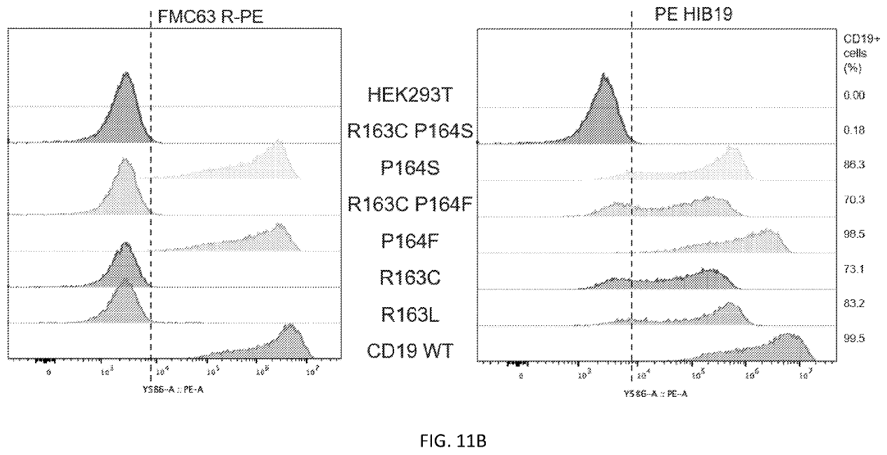

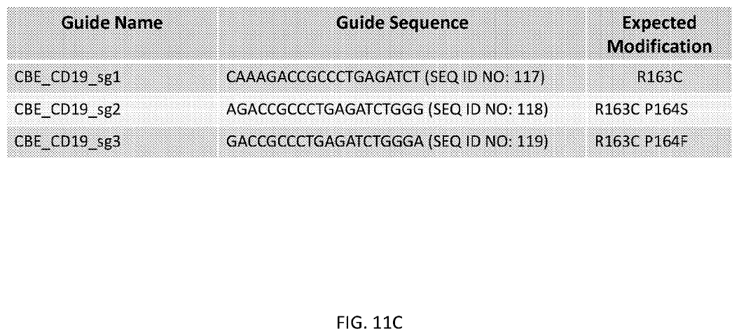

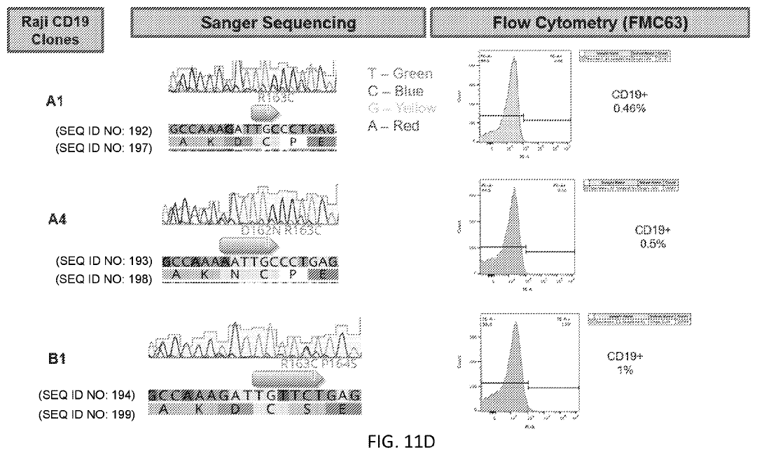

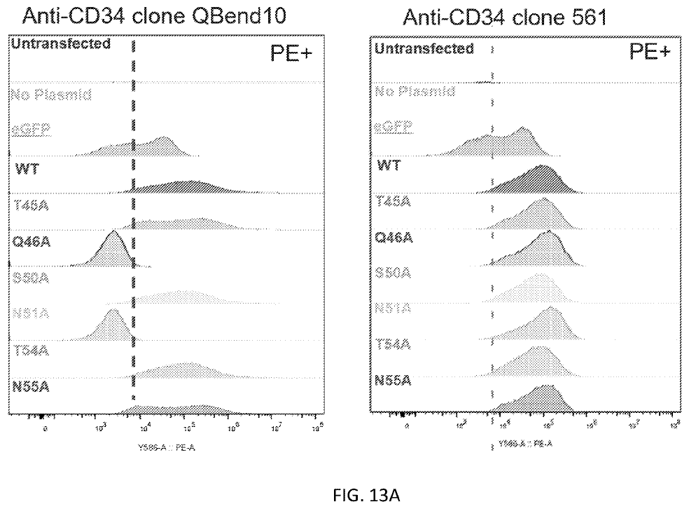

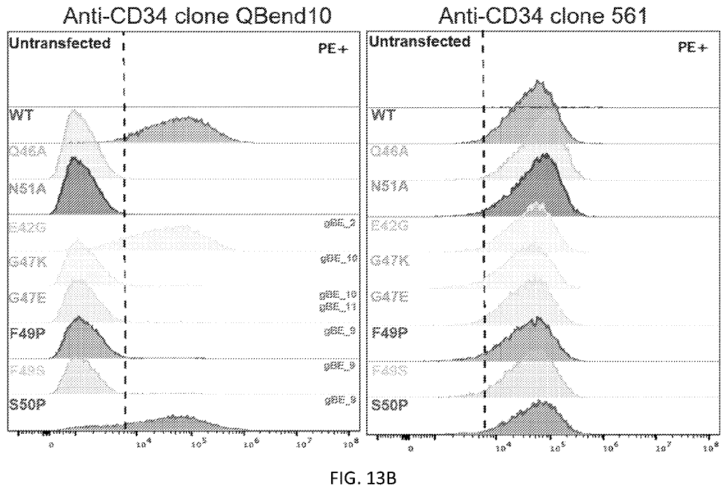

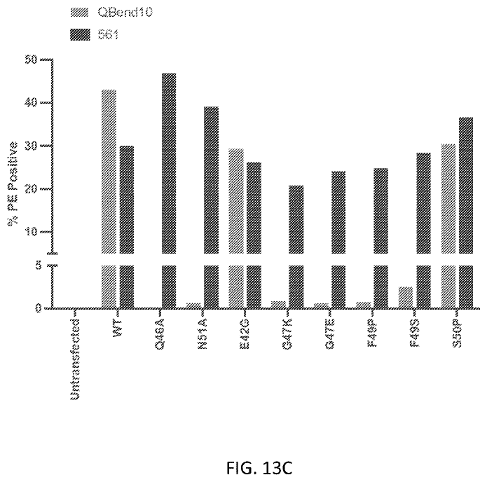

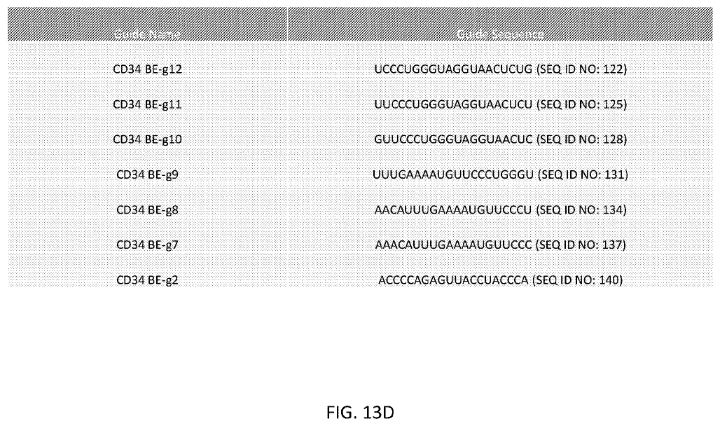

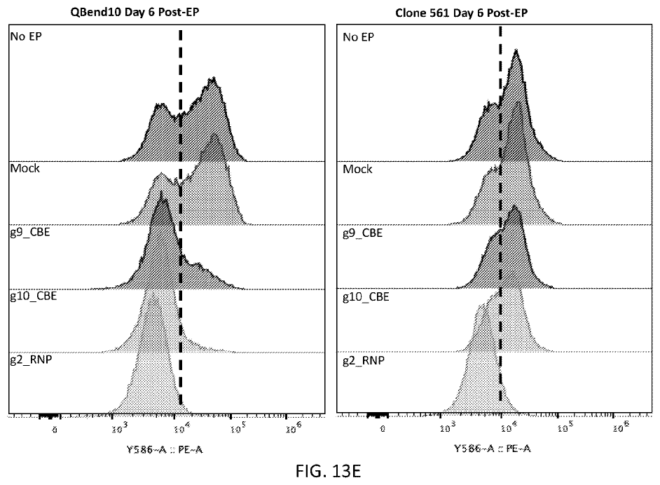

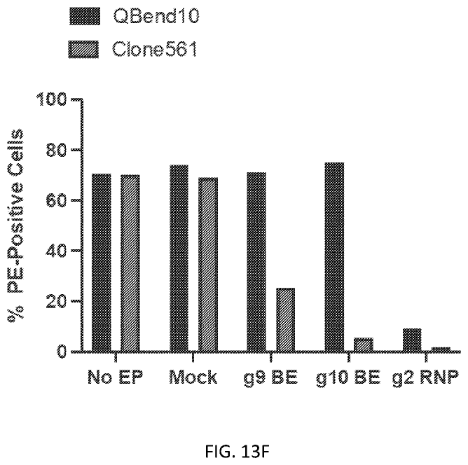

A collection of gRNAs was designed for use with either cytosine base editors (CBEs) or adenosine base editors (ABE) in order to generate epitope modifications in CD123 (see, e.g., Tables 1-3), CD38 (see, e.g., Tables 4-6), CD19 (see, e.g., FIG. 11C, Tables 7-9), CD34 (see, FIG. 13D, Table 13), CD5 (see, e.g., Table 12), and EMR2 (see, e.g., Tables 10-11) in cells (e.g., CD34+ cells such as HSPCs). The gRNAs required a PAM sequence, or were either “PAM-flexible” (i.e., comprising a PAM sequence “NG” and may be used with a base editor comprising, for example, SpG Cas9) or “PAM-less” (i.e., does not require a PAM sequence and may be used with a base editor comprising, for example, SpRY Cas9).

To determine CD19 epitopes to be modified with base editing, a screen was performed in which HEK293T cells were transfected with plasmids encoding different mutations in the CD19 protein. At 24 to 48 hours post-transfection, the HEK293T cells expressing wildtype CD19 or CD19 mutants were contacted with anti-CD19 antibody clones FMC63 and HIB 19 and analyzed via flow cytometry to determine which epitopes were required for antibody recognition. The results indicated that CD19 amino acid position R163, and amino acids proximal to this position, were involved in binding of the FMC63 clone but not substantially alter binding to the HIB19 clone (see FIG. 11A). Further analyses of HEK293T cells expressing CD19 mutations, in particular mutants containing changes to position R163 and proximal residues, indicated that variants comprising mutations at P163C+P164F, R163C, or P163L did not exhibit FMC63 binding, whereas recognition by the HIB19 was minimally affected (see FIG. 11B).

Based on the results from the epitope screening, gRNAs were designed for base editing the CD19 epitopes using CBE/ABE, as shown in Tables 7-9. For base editing, Raji cells were electroporated with the indicated gRNAs (CBE_CD19_sg1, CBE_CD19_sg2, CBE_CD19_sg3) (see FIG. 11C) and mRNA encoding a CBE. Single cell colonies were prepared from bulk edited cells and single cell clones were analyzed by Sanger sequencing and flow cytometry with the FMC63 antibody clone. The resulting cell clone populations showed different levels of CD19+ cells, however all the clone populations exhibited a drastic decrease in surface CD19 protein recognition by the FMC63 antibody (see FIG. 11D).

### CD47

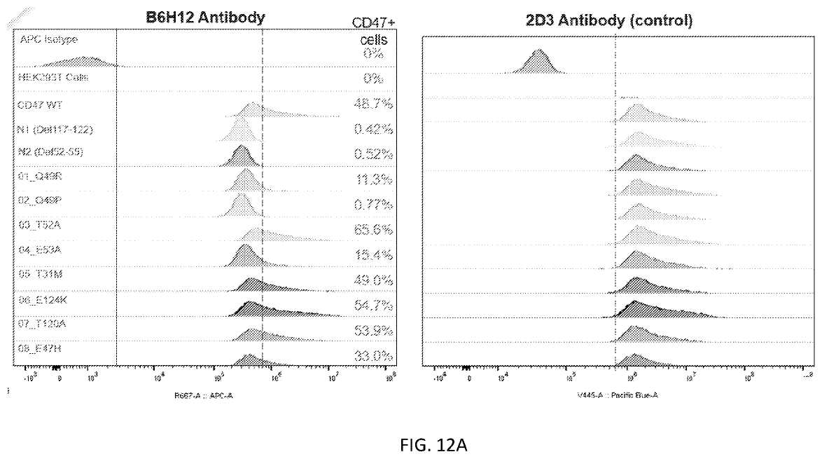

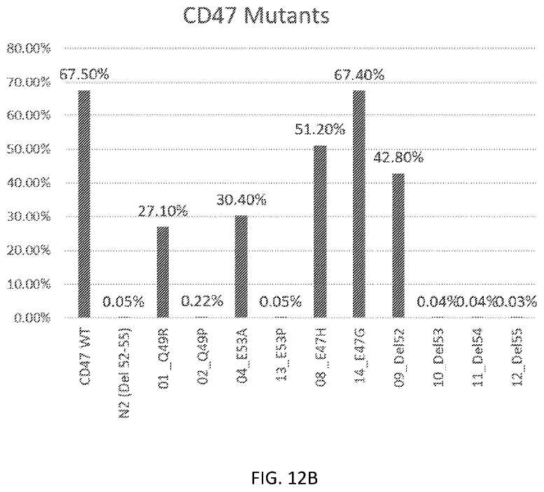

To determine CD47 epitopes that can be modified with base editing, a screen was performed in which HEK293T cells were transfected with plasmids encoding different mutations in the CD47 protein. At 24 to 48 hours post-transfection, HEK293T expressing wildtype CD47 or CD47 mutants were contacted with anti-CD47 antibody clones B6H12 and 2D3 and analyzed via flow cytometry to determine which epitopes were required for antibody recognition. The results indicated deletion of amino acids 117-122, amino acids 52-55, and substitution of Q49P drastically reduced binding by the B6H12 clone but not the 2D3 antibody clone. Additionally, Q49R, E53A, and E47H mutations partially reduced binding by the B6H12 clone but did not affect binding to the 2D3 clone. Also, T52A, T21M, E124K, and T120A mutations did not affect binding of either antibody clone (see, FIG. 12A).

CD47 epitopes involved in binding to the B6H12 antibody clone were further refined. HEK293T cells were transfected with plasmids encoding different mutations in the CD47 protein. At 24 to 48 hours post-transfection, flow cytometry was performed by contacting cells with the B6H12 antibody clone. CD19 containing Q49P or E53P mutations or deletion of the amino acids at position 53, 54, or 55 resulted in drastic reduction in antibody recognition, whereas Q49R and E53A partially reduced B6H12 clone binding (see FIG. 12B).

Based on the results from the epitope screening, gRNAs were designed for base editing the CD47 epitopes using CBE/ABE.

### CD34

To determine CD34 epitopes to be modified with base editing, a screen was performed in which HEK293T cells were transfected with plasmids encoding different mutations in the CD34 protein. At 24 to 48 hours post-transfection, HEK293T cells expressing wildtype CD34 or CD34 mutants were contacted with anti-CD34 antibody clones QBend10 and 561 and analyzed via flow cytometry to determine which epitopes were required for antibody recognition. The results indicated that CD34 Q46A and N51A mutations drastically reduced binding by the QBend10 antibody clone but did not affect binding to the 561 clone (see, FIG. 13A).

CD34 epitopes involved in binding to the QBend10 were further refined. HEK293T cells were transfected with plasmids encoding different mutations in the CD34 protein. At 24 to 48 hours post-transfection, flow cytometry was performed by contacting cells with QBend10 and 561 antibody clones. CD34 mutants Q46P, N51A, G47K, G47E, F49P, and F49S were each found to drastically reduce binding to the QBend10 antibody clone but not to the 561 clone (see, FIGS. 13B-13C).

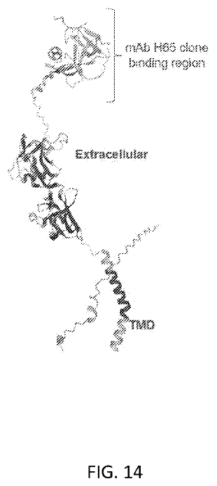

Based on the results from the epitope screening, gRNAs were designed for base editing the CD34 epitopes using CBE/ABE, as shown in FIG. 13D. CD34+ donor 2 cells were then modified via electroporation with gRNA of SEQ ID NO: 131 (CD34 BE-sg9), SEQ ID NO: 128 (CD34 BE-sg10), or SEQ ID NO: 140 (CD34 BE-sg2) (see FIG. 14D) and mRNA encoding the CBE (PpAPOBEC, BE4Max). Then, cells were contacted with either anti-CD34 antibody clone QBen10 or 561. As shown, in FIGS. 13E and 13F, the results indicated that base editing of CD34 using CD34 BE-sg9 and CD34 BE sg10 resulted in cells that had reduced binding to antibody QBend10, indicating that the epitope recognized by the antibody had been modified. Base editing cells with CD34 BE-sg2 resulted in reduced binding to both Qbend10 and 561 antibody clones, indicating epitope modification at recognition sites required by both antibodies.

### CD5

Analyses of the CD5 protein crystal structure were used to map target regions of CD5 domain 1 that bind to the anti-CD5 antibody clone H65 (see FIG. 14). A collection of gRNAs for use in the base editing methods described herein were designed for epitope modification of the H65 monoclonal antibody recognition sites in CD5 (see Table 12).

### EMR2

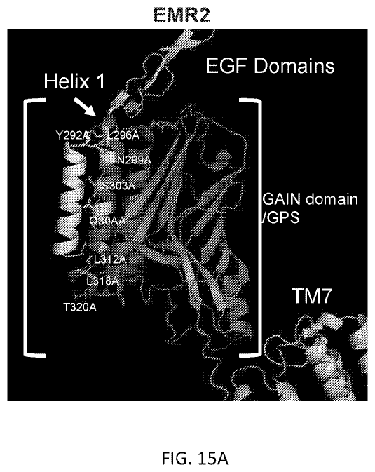

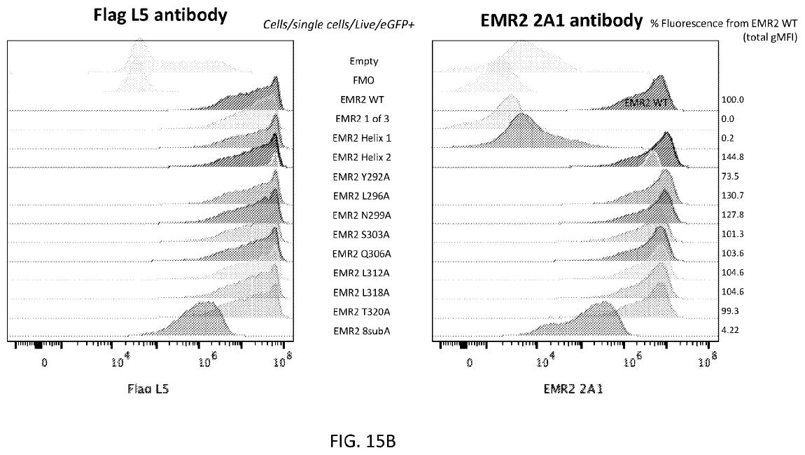

To determine EMR2 epitopes to be modified with base editing, a screen was performed in which HEK293T cells were transfected with plasmids encoding different mutations in the EMR2 protein. The EMR2 antibody clone 2A1 binding epitope for EMR2 was identified through deletion screen. Multiple regions of in EMR2 were deleted and ectopically expressed in HEK293T cells through plasmids. At 24 to 48 hours post-transfection, HEK293T cells expressing wildtype EMR2 or EMR2 mutants were contacted with anti-EMR2 clone 2A1 antibody or Flag L5 antibody and analyzed via flow cytometry to determine which epitopes were required for antibody recognition. Flowcytometry with the EMR2 clone 2A1 antibody showed that deletion of Helix 1 (amino acids 290-320) removed 2A1 antibody recognition (see FIG. 15B).

Based on the results from the epitope screening, gRNAs were designed for base editing the EMR2 epitopes using CBE/ABE, as shown in Tables 10 and 11.

### REFERENCES

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods described herein, can be excluded from any one or more claims. For purposes of brevity, all of the embodiments in which one or more elements, features, purposes, or aspects is excluded are not set forth explicitly herein.

What is claimed is:

1. A genetically engineered hematopoietic cell, or descendant thereof, comprising a genomic modification in a gene encoding a lineage-specific cell-surface antigen, wherein the genomic modification alters the amino acid sequence of an epitope that is recognized by an agent that specifically binds the lineage-specific cell-surface antigen resulting in a modified lineage-specific cell-surface antigen, and wherein the modified lineage-specific cell-surface antigen is characterized by reduced binding or no binding of the agent.

2. The genetically engineered hematopoietic cell, or descendant thereof, of claim 1, wherein the genomic modification alters 1, 2, 3, 4, or 5 amino acid residues of the lineage-specific cell-surface antigen.

3. The genetically engineered hematopoietic cell, or descendant thereof, of claim 1, wherein the genomic modification alters no more than 1, no more than 2, no more than 3, no more than 4, or no more than 5 amino acid residues of the lineage-specific cell-surface antigen.

4. The genetically engineered hematopoietic cell, or descendant thereof, of any one of claims 1-3, wherein the genomic modification results in a deletion, a substitution, an insertion, or an inversion of one or more amino acid residues, or a combination thereof.

5. The genetically engineered hematopoietic cell, or descendant thereof, of any one of claims 1-4, wherein the genomic modification results in a substitution of one or more amino acid residues.

6. The genetically engineered hematopoietic cell, or descendant thereof, of any one of claims 1-5, wherein the epitope is characterized by an endogenous post-translational modification.

7. The genetically engineered hematopoietic cell, or descendent thereof, of claim 6, wherein the endogenous post-translation modification is a glycosylation.

8. The genetically engineered hematopoietic cell, or descendant thereof, of any one of claims 1-7, wherein the agent is an immunotherapeutic agent.

9. The genetically engineered hematopoietic cell, or descendant thereof, of claim 8, wherein the immunotherapeutic agent comprises an antibody or an antigen-binding fragment thereof.

10. The genetically engineered hematopoietic cell, or descendant thereof, of any one of claims 1-7, wherein the modified lineage-specific cell-surface antigen is not recognized by the agent.

11. The genetically engineered hematopoietic cell, or descendant thereof, of any one of claims 1-8, wherein the modified lineage-specific cell-surface antigen is recognized by a second agent that specifically binds to a different region of the lineage-specific cell-surface antigen than the epitope recognized by the first agent.

12. The genetically engineered hematopoietic cell, or descendant thereof, of any one of claims 1-11, wherein the genomic modification does not substantially alter the function of the lineage-specific cell-surface antigen.

13. The genetically engineered hematopoietic cell, or descendant thereof, of any one of claims 1-12, wherein the genomic modification does not substantially alter the expression of the lineage-specific cell-surface antigen.

14. The genetically engineered hematopoietic cell, or descendant thereof, of any one of claims 1-13, wherein the genomic modification does not substantially alter the viability or growth of the cell.

15. The genetically engineered hematopoietic cell, or descendant thereof, of any one of claims 1-14, wherein the hematopoietic cell, or descendant thereof, retains the capacity to differentiate normally compared to a reference population of hematopoietic cells, optionally a population of hematopoietic cells not comprising the genomic modification.

16. The genetically engineered hematopoietic cell, or descendant thereof, of any one of claims 1-15, wherein the hematopoietic cell is a hematopoietic stem cell (HSC).

17. The genetically engineered hematopoietic cell, or descendant thereof, of any one of claims 1-16, wherein the hematopoietic cell is a CD34+ cell.

18. The genetically engineered hematopoietic cell, or descendant thereof, of any one of claims 1-17, wherein the hematopoietic cell is obtained from bone marrow, blood, umbilical cord, or peripheral blood mononuclear cells (PBMCs).

19. The genetically engineered hematopoietic cell, or descendant thereof, of any one of claims 1-18, wherein the hematopoietic cell is a human cell.

20. The genetically engineered hematopoietic cell, or descendant thereof, of any one of claims 1-17, wherein the lineage-specific cell-surface antigen is selected from the group consisting of CD123, CD47, CD34, CD38, CD19, CD33, CLL-1, CD30, CD5, CD6, CD7, EMR2, and BCMA.

21. The genetically engineered hematopoietic cell, or descendant thereof, of any one of claims 1-20, wherein the lineage-specific cell-surface antigen is CD123.

22. The genetically engineered hematopoietic cell, or descendant thereof, of any one of claims 1-20, wherein the lineage-specific cell-surface antigen is CD38.

23. The genetically engineered hematopoietic cell, or descendant thereof, of any one of claims 1-20, wherein the lineage-specific cell-surface antigen is CD19.

24. The genetically engineered hematopoietic cell, or descendant thereof, of any one of claims 1-20, wherein the lineage-specific cell-surface antigen is EMR2.

25. The genetically engineered hematopoietic cell, or descendant thereof, of any one of claims 1-20, wherein the lineage-specific cell-surface antigen is CD5.

26. The genetically engineered hematopoietic cell, or descendant thereof, of any one of claims 1-20, wherein the lineage-specific cell-surface antigen is CD47.

27. The genetically engineered hematopoietic cell, or descendant thereof, of any one of claims 1-20, wherein the lineage-specific cell-surface antigen is CD34.

28. The genetically engineered hematopoietic cell, or descendant thereof, of any one of claims 1-21, wherein the epitope is encoded by exon 3 and/or exon 4 of the gene encoding CD123.

29. The genetically engineered hematopoietic cell, or descendant thereof, of any one of claim 1-21 or 28, wherein the epitope is a region of CD123 bound by murine anti-CD123 antibody 7G3, a humanized variant thereof (e.g., antibody CSL-362), or talacotuzumab.

30. The genetically engineered hematopoietic cell, or descendant thereof, of any one of claim 1-21, 28, or 29, wherein the agent comprises murine anti-CD123 antibody 7G3, a humanized variant thereof (e.g., antibody CSL-362), talacotuzumab, or an antigen-binding fragment thereof.

31. The genetically engineered hematopoietic cell, or descendant thereof, of any one of claim 1-21 or 28-30, wherein the epitope comprises 1, 2, 3, 4, or 5 of the amino acids at positions 51, 59, 61, 82, or 84 of a wildtype gene encoding CD123.

32. The genetically engineered hematopoietic cell, or descendant thereof, of any one of claim 1-21 or 28-31, wherein the genomic modification results in a deletion, a substitution, an insertion, or an inversion of one or more of the amino acids at positions 51, 59, 61, 82, or 84 of a wildtype gene encoding CD123 or at corresponding positions in a homologous CD123 gene.

33. The genetically engineered hematopoietic cell, or descendant thereof, of any one of claim 1-21 or 28-32, wherein the genomic modification results in a substitution of one or more (e.g., 1, 2, 3, 4, or all) of the amino acids at positions 51, 59, 61, 82, or 84 of a wildtype gene encoding CD123 or at corresponding positions in a homologous CD123 gene.

34. The genetically engineered hematopoietic cell, or descendant thereof, of any one of claims 4-33, wherein the one or more substitutions are conservative substitutions.

35. The genetically engineered hematopoietic cell, or descendant thereof, of any one of claim 1-21 or 28-34, wherein the genomic modification results in a substitution of the amino acid at position 51 of a wildtype gene encoding CD123 or at a corresponding position in a homologous CD123 gene.

36. The genetically engineered hematopoietic cell, or descendant thereof, of any one of claim 1-21 or 28-34, wherein the genomic modification results in a substitution of a lysine for a glutamic acid at position 51 of a wildtype gene encoding CD123 or at a corresponding position in a homologous CD123 gene.

37. The genetically engineered hematopoietic cell, or descendant thereof, of any one of claim 1-20 or 22, wherein the epitope is encoded by exon 7 of the gene encoding CD38.

38. The genetically engineered hematopoietic cell, or descendant thereof, of any one of claim 1-20, 22, or 37, wherein the epitope is a region of CD38 bound by murine anti-CD38 antibody HB7, a humanized variant thereof, or daratumumab.

39. The genetically engineered hematopoietic cell, or descendant thereof, of any one of claim 1-20, 22, 37, or 38, wherein the agent comprises murine anti-CD38 antibody HB7, a humanized variant thereof, daratumumab, or an antigen-binding fragment thereof.

40. The genetically engineered hematopoietic cell, or descendant thereof, of any one of claim 1-20, 22, or 37-39, wherein the epitope comprises 1, 2, 3, 4, or 5 of the amino acids at positions 270-274 of a wildtype gene encoding CD38.

41. The genetically engineered hematopoietic cell, or descendant thereof, of any one of claim 1-20, 22, or 37-40, wherein the genomic modification results in a deletion, a substitution, an insertion, or an inversion of one or more of the amino acids at positions 270-274 of a wildtype gene encoding CD38 or at corresponding positions in a homologous CD38 gene.

42. The genetically engineered hematopoietic cell, or descendant thereof, of any one of claim 1-20, 22, or 37-41, wherein the genomic modification results in a substitution of one or more (e.g., 1, 2, 3, 4, or all) of the amino acids at positions 270-274 of a wildtype gene encoding CD38 or at corresponding positions in a homologous CD38 gene.

43. The genetically engineered hematopoietic cell, or descendant thereof, of any one of claim 4-20, 22, or 37-42, wherein the one or more substitutions are conservative substitutions.

44. The genetically engineered hematopoietic cell, or descendant thereof, of any one of claim 1-20, 22, or 37-43, wherein the genomic modification results in a substitution of the amino acid at position 272 of a wildtype gene encoding CD38 or at a corresponding position in a homologous CD38 gene.

45. The genetically engineered hematopoietic cell, or descendant thereof, of any one of claim 1-20, 22, or 37-44, wherein the genomic modification results in a substitution of an arginine, histidine, or alanine for glutamine at position 272 of a wildtype gene encoding CD38 or at a corresponding position in a homologous CD38 gene.

46. The genetically engineered hematopoietic cell, or descendant thereof, of any one of claim 1-20 or 23, wherein the epitope is encoded by exon 2 or exon 4 of CD19.

47. The genetically engineered hematopoietic cell, or descendant thereof, of any one of claim 1-20, 23, or 46, wherein the epitope is a region of CD19 bound by anti-CD19 antibody B43, anti-CD19 antibody FMC63, or an antigen-binding fragment thereof.

48. The genetically engineered hematopoietic cell, or descendant thereof, of any one of claim 1-20, 23, 46, or 47, wherein the agent comprises anti-CD19 antibody B43, anti-CD19 antibody FMC63, tafasitamab, loncastuximab, blinatumomab, or antigen-binding fragments thereof.

49. The genetically engineered hematopoietic cell, or descendant thereof, of any one of claim 1-20, 23, or 46-48, wherein the epitope comprises 1, 2, 3, 4, 5, 6, 7, 8, 9, 10, 11, 12, 13, 14, 15, 16, 17, 18, 19, or 20 of the amino acids at positions 216-224 or 218-238 of a wildtype gene encoding CD19.

50. The genetically engineered hematopoietic cell, or descendant thereof, of any one of claim 1-20, 23, or 46-49, wherein the genomic modification results in a deletion, a substitution, an insertion, or an inversion of one or more of the amino acids at positions 163, 164, 216-224, or 218-238 of a wildtype gene encoding CD19 or at corresponding positions in a homologous CD19 gene.

51. The genetically engineered hematopoietic cell, or descendant thereof, of any one of claim 1-20, 23, or 46-50, wherein the genomic modification results in a substitution of one or more (e.g., 1, 2, 3, 4, 5, 6, 7, 8, 9, 10, 11, 12, 13, 14, 15, 16, 17, 18, 19, 20, or more, e.g., all) of the amino acids at positions 163, 164, 216-224, or 218-238 of a wildtype gene encoding CD19 or at corresponding positions in a homologous CD19 gene.

52. The genetically engineered hematopoietic cell, or descendant thereof, of any one of claim 4-20, 23, or 46-51, wherein the one or more substitutions are conservative substitutions.

53. The genetically engineered hematopoietic cell, or descendant thereof, of any one of claim 1-20, 23, or 46-52, wherein the genomic modification results in a substitution of the amino acid at position 163 of a wildtype gene encoding CD19 or at a corresponding position in a homologous CD19 gene.

54. The genetically engineered hematopoietic cell, or descendant thereof, of any one of claim 1-20, 23, or 46-53, wherein the genomic modification results in a substitution of the amino acid at position 163 and 220 of a wildtype gene encoding CD19 or at a corresponding position in a homologous CD19 gene.

55. The genetically engineered hematopoietic cell, or descendant thereof, of any one of claim 1-20, 23, or 46-53, wherein the genomic modification results in a substitution of the amino acid at position 163 and 164 of a wildtype gene encoding CD19 or at a corresponding position in a homologous CD19 gene.

56. The genetically engineered hematopoietic cell, or descendant thereof, of any one of claim 1-20, 23, or 46-53, wherein the genomic modification results in a substitution of a cysteine or a leucine at the amino acid at position 163 of a wildtype gene encoding CD19 or at a corresponding position in a homologous CD19 gene.

57. The genetically engineered hematopoietic cell, or descendant thereof, of any one of claim 1-20, 23, or 46-53, wherein the genomic modification results in a substitution of a phenylalanine at the amino acid at position 164 of a wildtype gene encoding CD19 or at a corresponding position in a homologous CD19 gene.

58. The genetically engineered hematopoietic cell, or descendant thereof, of any one of claim 1-20, 23, or 46-53, wherein the genomic modification results in a substitution of the amino acid at position 163 and 164 of a wildtype gene encoding CD19 or at a corresponding position in a homologous CD19 gene, wherein the substitution of the amino acid at position 163 is a cysteine or a leucine and the substitution of the amino acid at position 164 is a phenylalanine.

59. The genetically engineered hematopoietic cell, or descendant thereof, of any one of claims 1-20 and 24, wherein the epitope comprises 1, 2, 3, 4, 5, or 6 of the amino acids at positions 124, 132, 146, 292, 294, 295, 296, 298, 299, 303, 304, 305, 306, 307, 308, 312, 318, 320, 328, 329, 331, 332, 335, 340, 347, 527, or 708 of a wildtype gene encoding EMR2.

60. The genetically engineered hematopoietic cell, or descendant thereof, of any one of claim 1-20, 24, or 59, wherein the genomic modification results in a deletion, a substitution, an insertion, or an inversion of one or more of the amino acids at positions 124, 132, 146, 292, 294, 295, 296, 298, 299, 303, 304, 305, 306, 307, 308, 312, 318, 328, 329, 331, 332, 335, 340, 347, 527, or 708 of a wildtype gene encoding EMR2 or at corresponding positions in a homologous EMR2 gene.

61. The genetically engineered hematopoietic cell, or descendant thereof, of any one of claim 1-20, or 26, wherein the epitope is a region of CD47 bound by anti-CD47 antibody B6H12, anti-CD47 antibody 2D3, or antigen-binding fragments thereof.

62. The genetically engineered hematopoietic cell, or descendant thereof, of any one of claim 1-20, 26, or 61, wherein the agent comprises anti-CD47 antibody B6H12, anti-CD47 antibody 2d3, Ligufalimab, or antigen-binding fragments thereof.

63. The genetically engineered hematopoietic cell, or descendant thereof, of any one of claim 1-20, 26, 61, or 62, wherein the epitope comprises 1, 2, 3, 4, 5, or 6 of the amino acids at positions 117-122 of a wildtype gene encoding CD47.

64. The genetically engineered hematopoietic cell, or descendant thereof, of any one of claim 1-20, 26, or 61-63, wherein the epitope comprises 1, 2, 3, or 4 of the amino acids at positions 47, 49, 52-55 or 117-122 of a wildtype gene encoding CD47.

65. The genetically engineered hematopoietic cell, or descendant thereof, of any one of claim 1-20, 26, or 61-64, wherein the genomic modification results in a deletion, a substitution, an insertion, or an inversion of one or more of the amino acids at positions 31, 47, 49, 52-55, 117-122, or 124 of a wildtype gene encoding CD47 or at corresponding positions in a homologous CD47 gene.

66. The genetically engineered hematopoietic cell, or descendant thereof, of any one of claim 4-20, 26, or 61-65, wherein the one or more substitutions are conservative substitutions.

67. The genetically engineered hematopoietic cell, or descendant thereof, of any one of claim 1-20, 26, or 61-66, wherein the genomic modification results in a substitution of one or more of the amino acids at positions 31, 47, 49, 52-55 117-122, or 124 of a wildtype gene encoding CD47 or at a corresponding position in a homologous CD47 gene.

68. The genetically engineered hematopoietic cell, or descendant thereof, of any one of claim 1-20, 26, or 61-67, wherein the genomic modification results in a substitution of the amino acid at position 49 of a wildtype gene encoding CD47 or at a corresponding position in a homologous CD47 gene.

69. The genetically engineered hematopoietic cell, or descendant thereof, of any one of claim 1-20, 26, or 61-68, wherein the genomic modification results in a substitution of
(i) a histidine at the amino acid at position 4,
(ii) an arginine at the amino acid at position 49,
(iii) a proline at the amino acid at position 49,
(iv) an alanine at the amino acid at position 52,
(v) an alanine at the amino acid at position 53,
(vi) a proline at the amino acid at position 53,
(v) an alanine at the amino acid at position 120, or
(vi) a lysine at the amino acid at position 124;
of a wildtype gene encoding CD47 or at a corresponding position in a homologous CD47 gene.

70. The genetically engineered hematopoietic cell, or descendant thereof, of any one of claim 1-20 or 27, wherein the epitope is a region of CD34 bound by anti-CD34 antibody QBend10, anti-CD34 antibody 561, or antigen-binding fragments thereof.

71. The genetically engineered hematopoietic cell, or descendant thereof, of any one of claim 1-20, 27, or 70, wherein the genomic modification results in a deletion, a substitution, an insertion, or an inversion of one or more of the amino acids at positions 42, 45, 46, 47, 49, 50, 51, 54, or 55 of a wildtype gene encoding CD34 or at corresponding positions in a homologous CD34 gene.

72. The genetically engineered hematopoietic cell, or descendant thereof, of any one of claim 4-20, 27, 70, or 71, wherein the one or more substitutions are conservative substitutions.

73. The genetically engineered hematopoietic cell, or descendant thereof, of any one of claim 1-20, 27, or 70-72, wherein the genomic modification results in a substitution of one or more of the amino acids at positions 42, 45, 46, 47, 49, 50, 51, 54, or 55 of a wildtype gene encoding CD34 or at corresponding positions in a homologous CD34 gene.

74. The genetically engineered hematopoietic cell, or descendant thereof, of any one of claim 1-20, 27, or 70-73, wherein the genomic modification results in a substitution of an alanine at the amino acid at any one or more of positions 45, 46, 50, 51, 54, 55 of a wildtype gene encoding CD34 or at a corresponding position in a homologous CD34 gene.

75. The genetically engineered hematopoietic cell, or descendant thereof, of any one of claim 1-20, 27, or 70-74, wherein the genomic modification results in a substitution of
(i) phenylalanine at the amino acid of position 46,
(ii) lysine at the amino acid of position 47,
(iii) glutamic acid at the amino acid position 47,
(iv) phenylalanine at amino acid position 49, or
(v) serine at amino acid position 49;
of a wildtype gene encoding CD34 or at a corresponding position in a homologous CD34 gene.

76. A method, comprising administering to a subject in need thereof:
(i) a population of the genetically engineered hematopoietic cells, or descendants thereof, of any one of claims 1-75.

77. The method of claim 76, further comprising administering (ii) an effective amount of an agent that specifically binds the lineage-specific cell-surface antigen.

78. The method of claim 76 or 77, wherein the subject has a hematopoietic malignancy.

79. The method of claim 77 or 78, wherein the agent is a single-chain antibody fragment (scFv).

80. The method of any one of claims 77-79, wherein the agent is an antibody or an antibody-drug conjugate (ADC).

81. The method of claim 77 or 78, wherein the agent is an immune cell expressing a chimeric antigen receptor that comprises an antigen-binding fragment.

82. The method of claim 81, wherein the immune cells are T cells.

83. The method of claim 82, wherein the T cells express CD3, CD4, and/or CD8.

84. The method of any one of claims 81-83, wherein the chimeric antigen receptor further comprises:
(a) a hinge domain,
(b) a transmembrane domain,
(c) at least one co-stimulatory domain,
(d) a cytoplasmic signaling domain, or
(e) a combination thereof.

85. The method of claim 84, wherein the chimeric antigen receptor comprises at least one co-stimulatory signaling domain, which is derived from a co-stimulatory receptor selected from the group consisting of CD27, CD28, 4-1BB, OX40, CD30, ICOS, CD2, CD7, LIGHT, NKG2C, B7-H3, GITR, HVEM, and a combination thereof.

86. The method of claim 84 or claim 85, wherein the chimeric antigen receptor comprises a cytoplasmic signaling domain, which is from CD3ζ.

87. The method of any one of claims 84-86, wherein the chimeric antigen receptor comprises a hinge domain, which is from CD8α or CD28.

88. The method of any one of claims 77-87, wherein the agent comprises: murine anti-CD123 antibody 7G3, a humanized variant thereof (e.g., antibody CSL-362), or talacotuzumab; murine anti-CD38 antibody HB7, a humanized variant thereof, or daratumumab; B43; or antiCD19 antibody blinatumomab, FMC63, or HIB19; or anti-CD47 antibody B6H12 or 2D3; or anti-CD34 antibody QBend10 or 561; or anti-CD5 antibody H65.

89. The method of any one of claims 78-88, wherein the hematopoietic malignancy is Hodgkin's lymphoma, non-Hodgkin's lymphoma, leukemia, multiple myeloma (MM), myelodysplastic syndrome (MDS), or blastic plasmacytoid dendritic cell neoplasm (BPDCN).

90. The method of any one of claims 78-89, wherein the hematopoietic malignancy is acute myeloid leukemia, B-cell acute lymphoblastic leukemia (B-ALL), chronic myelogenous leukemia, acute lymphoblastic leukemia, or chronic lymphoblastic leukemia.

91. The method of any one of claims 78-90, wherein the hematopoietic malignancy is B-cell acute lymphoblastic leukemia (B-ALL).

92. The method of any one of claims 78-90, wherein the hematopoietic malignancy is acute myeloid leukemia (AML).

93. The method of any one of claims 78-90, wherein the hematopoietic malignancy is multiple myeloma (MM).

94. The method of any one of claims 78-90, wherein the hematopoietic malignancy is myelodysplastic syndrome (MDS).

95. A method comprising:
genetically modifying a hematopoietic cell to introduce a genomic modification in a gene encoding a lineage-specific cell-surface antigen, wherein the genomic modification alters the amino acid sequence of an epitope that is recognized by an agent that specifically binds the lineage-specific cell-surface antigen resulting in a modified lineage-specific cell surface antigen, wherein the modified lineage-specific cell-surface antigen is characterized by reduced binding or no binding of the agent,
thereby producing a genetically engineered hematopoietic cell having reduced binding or no binding to an agent targeting the lineage-specific cell-surface antigen.

96. The method of claim 95, further comprising:
providing a hematopoietic cell.

97. The method of claim 95 or 96, wherein the genetically engineered hematopoietic cell is a genetically engineered hematopoietic cell of any one of claims 1-75.

98. The method of any one of claims 95-97, wherein genetically modifying the hematopoietic cell comprises contacting the cell with:
(a) a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a gene encoding a lineage-specific cell-surface antigen (e.g., a sequence encoding an epitope bound by an agent that specifically binds the lineage-specific cell-surface antigen) in the genome of the hematopoietic cell; and
(b) a template polynucleotide.

99. The method of claim 98, wherein the contacting further comprises contacting the hematopoietic cell with:
(c) one or both of:
(i) an expansion agent;
(ii) a homology-directed repair (HDR) promoting agent.

100. The method of either one of claim 98 or 99, wherein the CRISPR/Cas system creates a double-stranded break (DSB) in the gene encoding the lineage-specific cell-surface antigen in the genome of the hematopoietic cell.

101. The method of any one of claims 98-100, wherein the template polynucleotide is a single-stranded donor oligonucleotide (ssODN) or a double-stranded donor oligonucleotide (dsODN).

102. The method of any one of claims 98-101 wherein the template polynucleotide hybridizes to a genomic sequence flanking the DSB in the gene encoding the lineage-specific cell-surface antigen and integrates into the gene encoding the lineage-specific cell-surface antigen.

103. The method of any one of claims 98-102, wherein the template polynucleotide comprises a donor sequence, a first flanking sequence which is homologous to a genomic sequence upstream of the DSB in the gene encoding the lineage-specific cell-surface antigen and a second flanking sequence which is homologous to a genomic sequence downstream of the DSB in the gene encoding the lineage-specific cell-surface antigen.

104. The method of claim 103, wherein the donor sequence of the template polynucleotide is integrated into the genome of the hematopoietic cell by homology-directed repair (HDR).

105. The method of any one of claims 99-104, wherein the expansion agent comprises SR1 and UM171.

106. The method of any one of claims 99-105, wherein the HDR promoting agent comprises at least one of SCR7, NU7441, Rucaparib, and RS-1.

107. The method of any one of claims 101-106, wherein the ssODN is between 50 to 200 nucleotides in length.

108. The method of any one of claims 101-107, wherein the ssODN is 120 nucleotides in length.

109. The method of any one of claims 98-108, wherein contacting comprises contacting a population of hematopoietic cells.

110. The method of claim 109, further comprising sorting the population of hematopoietic cells.

111. The method of claim 110, wherein sorting comprises selecting for viable hematopoietic cells.

112. The method of claim 110 or 111, wherein sorting comprises selecting for hematopoietic cells that integrated the donor sequence into their genome.

113. The method of any one of claims 110-112, wherein sorting comprises Fluorescence Activated Cell Sorting (FACS).

114. The method of any one of claims 110-113, wherein sorting comprises selecting for viable long term engrafting HSCs.

115. The method of any one of claims 110-114, wherein the editing efficiency in the population of hematopoietic cells is at least 30%, at least 40%, at least 50%, at least 60%, at least 70%, at least 80%, at least 90%, at least 95%, or at least 99%.

116. The method of any one of claims 110-115, wherein the percent viability in the population of hematopoietic cells is at least 50%, at least 60%, at least 70%, at least 80%, at least 90%, at least 95%, or at least 99%.

117. The method of any one of claims 110-116, wherein the efficiency of HDR is 50% or higher.

118. The method of any one of claims 110-117, wherein the efficiency of HDR is 60% or higher.

119. The method of any one of claims 110-118, wherein the efficiency of HDR is 80% or higher.

120. The method of any one of claims 95-119, wherein the lineage-specific cell-surface antigen is selected from the group consisting of CD33, CD123, CD19, CLL-1, CD30, CD5, EMR2, CD6, CD7, CD38, CD34, CD47, and BCMA.

121. The method of any one of claims 95-120, wherein the lineage-specific cell-surface antigen is CD123.

122. The method of claim 121, wherein the gRNA comprises a nucleic acid sequence selected from the group consisting of SEQ ID NOs: 3, 6, 9, and 12.

123. The method of claim 121 or 122, wherein the first flanking sequence is homologous to a first portion of the CD123 gene and the second flanking sequence is homologous to a second portion of the CD123 gene.

124. The method of claim 123, wherein the first portion of the CD123 gene comprises a portion of exon 3 or a sequence proximal thereto.

125. The method of claim 123, wherein the first portion of the CD123 gene comprises a portion of exon 4 or a sequence proximal thereto.

126. The method of any one of claims 123-125, wherein the second portion of the CD123 gene comprises a portion of exon 3 or a sequence proximal thereto.

127. The method of any one of claims 123-125, wherein the second portion of the CD123 gene comprises a portion of exon 4 or a sequence proximal thereto.

128. The method of any one of claims 123-127, wherein the first portion and second portion are not identical.

129. The method of any one of claims 121-128, wherein the donor sequence comprises a sequence corresponding to the codon(s) encoding 1, 2, 3, 4, or 5 of the amino acids at positions 51, 59, 61, 82, or 84 of a wildtype gene encoding CD123.

130. The method of any one of claims 121-129, wherein the first flanking sequence comprises a flanking sequence set forth in any one of SEQ ID NOs: 93-99.

131. The method of any one of claims 121-130, wherein the second flanking sequence comprises a flanking sequence set forth in any one of SEQ ID NOs: 93-99.

132. The method of any one of claims 121-131, wherein the donor sequence comprises a donor sequence set forth in any one of SEQ ID NOs: 93-99.

133. The method of any one of claims 121-132, wherein the template polynucleotide comprises a nucleic acid sequence selected from the group consisting of SEQ ID NOs: 93-99.

134. The method of any one of claims 95-120, wherein the lineage-specific cell-surface antigen is CD38.

135. The method of claim 134, wherein the gRNA comprises a nucleic acid sequence selected from the group consisting of SEQ ID NOs: 18, 21, 24, 27, 30, 33, 36, 39, 42, 45, 48, 51, 54, 57, and 60.

136. The method of claim 134 or 135, wherein the first flanking sequence is homologous to a first portion of the CD38 gene and the second flanking sequence is homologous to a second portion of the CD38 gene.

137. The method of claim 136, wherein the first portion of the CD38 gene comprises a portion of exon 7 or a sequence proximal thereto.

138. The method of claim 136 or 137, wherein the second portion of the CD38 gene comprises a portion of exon 7 or a sequence proximal thereto.

139. The method of any one of claims 136-138, wherein the first portion and second portion are not identical.

140. The method of any one of claims 134-139, wherein the donor sequence comprises a sequence corresponding to the codon(s) encoding 1, 2, 3, 4, or 5 of the amino acids at positions 270-274 of a wildtype gene encoding CD38.

141. The method of any one of claims 95-120, wherein the lineage-specific cell-surface antigen is CD19.

142. The method of claim 141, wherein the gRNA comprises a nucleic acid sequence selected from the group consisting of SEQ ID NOs: 66, 69, 72, 75, 78, 81, and 84.

143. The method of claim 141 or 142, wherein the first flanking sequence is homologous to a first portion of the CD19 gene and the second flanking sequence is homologous to a second portion of the CD19 gene.

144. The method of claim 143, wherein the first portion of the CD19 gene comprises a portion of exon 2 or a sequence proximal thereto.

145. The method of claim 143, wherein the first portion of the CD19 gene comprises a portion of exon 4 or a sequence proximal thereto.

146. The method of any one of claims 143-145, wherein the second portion of the CD19 gene comprises a portion of exon 2 or a sequence proximal thereto.

147. The method of any one of claims 143-145, wherein the second portion of the CD19 gene comprises a portion of exon 4 or a sequence proximal thereto.

148. The method of any one of claims 143-147, wherein the first portion and second portion are not identical.

149. The method of any one of claims 141-148, wherein the donor sequence comprises a sequence corresponding to the codon(s) encoding 1, 2, 3, 4, 5, 6, 7, 8, 9, 10, 11, 12, 13, 14, 15, 16, 17, 18, 19, or 20 of the amino acids at positions 216-224 or 218-238 of a wildtype gene encoding CD19.

150. The method of any one of claims 95-149, wherein the genomic modification results in expression of a variant form of the lineage-specific cell surface antigen that is not recognized by the agent.

151. The method of any one of claims 95-150, wherein the genomic modification results in expression of a variant form of the lineage-specific cell surface antigen that is recognized by a second agent that specifically binds to a different region of the lineage-specific cell-surface antigen than the agent that binds the epitope.

152. The method of any one of claims 96-151, wherein the Cas nuclease is a Cas9 nuclease.

153. The method of any one of claims 96-152, wherein the Cas nuclease is a Streptococcus pyogenes Cas9 (spCas9) nuclease.

154. The method of any one of claims 96-152, wherein the Cas nuclease is a Staphylococcus aureus Cas9 (saCas9) nuclease.

155. The method of any one of claims 96-152, wherein the Cas nuclease is a Cas12a nuclease.

156. The method of any one of claims 96-152, wherein the Cas nuclease is a Cas12b nuclease.

157. The method of any one of claims 96-156, wherein the contacting comprises introducing the CRISPR/Cas system into the cell in the form of a pre-formed ribonucleoprotein (RNP) complex.

158. The method of claim 157, wherein the ribonucleoprotein complex is introduced into the hematopoietic cell via electroporation.

159. The method of any one of claims 98-158, wherein the template polynucleotide and CRISPR/Cas system are electroporated into the cell simultaneously.

160. A genetically engineered hematopoietic cell, where the cell is obtained or obtainable by the method of any one of claims 95-159.

161. A population of genetically engineered hematopoietic cells comprising a plurality of the genetically engineered hematopoietic cells of any one of claims 1-75 or the genetically engineered hematopoietic cell of claim 160.

162. A pharmaceutical composition comprising the genetically engineered hematopoietic cell, or descendant thereof, of any one of claims 1-75, the genetically engineered hematopoietic cell of claim 160, or the population of genetically engineered hematopoietic cells of claim 161.

163. A method of producing a genetically engineered hematopoietic stem or progenitor cell, or a plurality thereof, comprising at least one nucleotide substitution in a gene encoding a lineage-specific cell surface antigen, wherein the method comprises introducing into a hematopoietic stem or progenitor cell:
(i) a guide RNA (gRNA) comprising a targeting domain targeting a nucleotide sequence within the genome of the hematopoietic stem or progenitor cell; and
(ii) a base editor comprising a catalytically impaired Cas9 endonuclease fused to a cytosine (CBE) or adenosine deaminase (CBE), thereby producing the genetically engineered hematopoietic stem or progenitor cell or a plurality thereof.

164. The method of claim 163, wherein the at least one substitution produces a missense variant in the gene encoding the lineage-specific cell-surface antigen.

165. The method of claim 163, wherein the at least one substitution produces an alteration in the translation start site of the gene encoding the lineage-specific cell-surface antigen.

166. The method of claim 163, wherein the at least one substitution produces a splice region variant in the gene encoding the lineage-specific cell-surface antigen.

167. The method of any one of claims 163-166, wherein the gene encoding the lineage-specific cell-surface antigen is selected from the group consisting of CD123, CD47, CD34, CD38, CD19, CD33, CLL-1, CD30, CD5, CD6, CD7, and BCMA.

168. The method of any one of claims 163-167, wherein the gene encoding the lineage-specific cell-surface antigen is selected from the group consisting of CD123, CD47, CD34, CD38, CD19, and CD5.

169. The method of any one of claims 163-168, wherein the gene encoding the lineage-specific cell-surface antigen is CD123.

170. The method of any one of claims 163-169, wherein the gene encoding the lineage-specific cell-surface antigen is CD47.

171. The method of any one of claims 163-169, wherein the gene encoding the lineage-specific cell-surface antigen is CD34.

172. The method of any one of claims 163-169, wherein the gene encoding the lineage-specific cell-surface antigen is CD38.

173. The method of any one of claims 163-169, wherein the gene encoding the lineage-specific cell-surface antigen is CD19.

174. The method of any one of claims 163-169, wherein the gene encoding the lineage-specific cell-surface antigen is CD5.

175. The method of any one of claims 163-174, wherein the gRNA comprises a nucleotide sequence set forth in any one of SEQ ID NOs: 1-12, 16-60, 64-84, 100-181, 195, 196, and 204-423.

176. The method of any one of claims 163-175, wherein the catalytically impaired Cas9 nuclease is a SpRY Cas9.

177. The method of any one of claims 163-175, wherein the catalytically impaired Cas9 nuclease is a SpG Cas9.

178. The method of any one of claims 163-177, wherein the base editor is introduced into the cell as an mRNA.

179. The method of any one of claims 163-178, wherein the base editor and gRNA are introduced into the cell via electroporation.

180. The method of any one of claims 163-179, wherein the method further comprises sorting the genetically engineered hematopoietic stem or progenitor cell, or plurality thereof, via fluorescence-activated cell sorting (FACS).

181. The method of any one of claims 163-180, wherein the substitution results in reduced or eliminated expression of a gene encoding a wild-type version of the lineage-specific cell-surface antigen.

182. A genetically engineered hematopoietic stem or progenitor cell produced by the method of any one of claims 163-181.

183. A cell population comprising a plurality of the genetically engineered hematopoietic stem or progenitor cell of claim 182.

184. A pharmaceutical composition comprising the genetically engineered hematopoietic stem or progenitor cell of claim 182 or the cell population of claim 183.

185. A method of treating a hematopoietic disease, comprising administering to a subject in need thereof an effective amount of the genetically engineered hematopoietic stem or progenitor cell of claim 182, the cell population of claim 183, or the pharmaceutical composition of claim 184.

186. The method of claim 185, wherein the hematopoietic disease is a hematopoietic malignancy.

187. The method of claim 185 or 186, wherein the method further comprises administering an effective amount of an agent that targets a wildtype version of lineage-specific cell-surface antigen.

188. The method of claim 87, wherein the agent comprises an antibody or antigen-binding fragment that binds to the wildtype version of the lineage-specific cell-surface antigen.

189. The method of claim 188, wherein the agent is an immune cell.

190. The method of claim 189, wherein the immune cell is a cytotoxic T cell.

191. The method of claim 190, wherein the cytotoxic T cell expresses a chimeric antigen receptor (CAR) which comprises the antibody or antigen-binding fragment that binds the wildtype version of the lineage-specific cell-surface antigen.

192. The method of any one of claims 188-191, wherein the antibody is selected from the group consisting of a anti-CD123 antibody 7G3, talacotuzumab, anti-CD38 antibody HB7, daratumumab, anti-CD38 antibody B43, blinatumomab, anti-CD19 antibody FMC63, anti-CD19 antibody HIB19, anti-CD47 antibody B6H12, anti-CD47 antibody 2D3, anti-CD34 antibody QBend10, anti-CD34 antibody 561, and anti-CD5 antibody H65.

193. The method of any one of claims 185-192, wherein the genetically engineered hematopoietic stem or progenitor cell, the immune cell, or both, are allogenic.

194. The method of any one of claims 185-193, wherein the genetically engineered hematopoietic stem or progenitor cell, the immune cell, or both, are autologous.

195. The method of any one of claims 185-194, wherein the subject is a human patient having Hodgkin's lymphoma, non-Hodgkin's lymphoma, leukemia, acute myeloid leukemia (AML), chronic myelogenous leukemia, acute lymphoblastic leukemia, or chronic lymphoblastic leukemia.


SEQUENCE LISTING (425 sequences)

SEQ ID NO: 1 - DNA, 20 bp, Homo sapiens
  source 1..20: mol_type = genomic DNA
cacatttctgttaaggtccc

SEQ ID NO: 2 - DNA, 20 bp, Homo sapiens
  source 1..20: mol_type = genomic DNA
gggaccttaacagaaatgtg

SEQ ID NO: 3 - RNA, 20 nt, Homo sapiens
  source 1..20: mol_type = genomic RNA
cacatttctgttaaggtccc

SEQ ID NO: 4 - DNA, 20 bp, Homo sapiens
  source 1..20: mol_type = genomic DNA
tatcggtcacatttctgtta

SEQ ID NO: 5 - DNA, 20 bp, Homo sapiens
  source 1..20: mol_type = genomic DNA
taacagaaatgtgaccgata

SEQ ID NO: 6 - RNA, 20 nt, Homo sapiens
  source 1..20: mol_type = genomic RNA
tatcggtcacatttctgtta

SEQ ID NO: 7 - DNA, 20 bp, Homo sapiens
  source 1..20: mol_type = genomic DNA
gtctttaacacactcgatat

SEQ ID NO: 8 - DNA, 20 bp, Homo sapiens
  source 1..20: mol_type = genomic DNA
atatcgagtgtgttaaagac

SEQ ID NO: 9 - RNA, 20 nt, Homo sapiens
  source 1..20: mol_type = genomic RNA
gtctttaacacactcgatat

SEQ ID NO: 10 - DNA, 20 bp, Homo sapiens
  source 1..20: mol_type = genomic DNA
agacgccgactattctatgc

SEQ ID NO: 11 - DNA, 20 bp, Homo sapiens
  source 1..20: mol_type = genomic DNA
gcatagaatagtcggcgtct

SEQ ID NO: 12 - RNA, 20 nt, Homo sapiens
  source 1..20: mol_type = genomic RNA
agacgccgactattctatgc

SEQ ID NO: 13 - DNA, 32767 bp, Homo sapiens
  source 1..32767: mol_type = genomic DNA
aacatgataattttcaaagaaagagtctttctttcaaggaaagtcaggttcatggttacgaagctgctgaccccaggatcccagcccgtgggagagaagggggtctctgacagcccccacccctccccactgccagatccttattgggtctgagtttcaggggtggggccccagctggaggttataaaacagctcaatcggggagtacaaccttcggtttctcttcggggaaagctgctttcagcgcacacgggaagatatcagaaacatcctaggatcaggacaccccagatcttctcaactggaaccacgaaggctgtttcttccacacagtactttgatctccatttaaggtaaggtcccccctccagggtgggatgagggaaaaagagggcaggcaggggaggcggtggactgtgggttcccaaatccaagctggcagacacggggcattggcatgtaacaggtttctgctgagacctgctgtccgtgtgggcaccacaccacacatagaatgacccggtaccagagaagtatgtttgcgatctcattcacccttttgtaagtggatcggacgctgaagccatcatagcatgttagatgccttgggtttataaaggtgatagcattaggagtaatttgacagtgctttctgctttttctgtgtgcttgctagaaaatgtacacacctacacatggctagagttctctgtttattagaaagcgctggttcagttagtcaatgagctcacgcccagggcgagctgactgccagccggggagccgcttttggggctgggaccccagcctgcttgtagtgtttctcagtggttttcaaaggctggcgggactcagaatgttccagatggctggtgaaacctcaggtccctcctagcagctttattcacagtagccaagaggtggagacagccctcgtacccatctatagatgaatggaaaaatataatgtccatccacacagtggaatattacgcagccacgtttctcctgcagcaggcacctctgtcctgcgttccggagctgcgttcccgatggtcctcctttggctcacgctgctcctgatcgccctgccctgtctcctgcaaacgaaggaaggtaagaactggagaaaaaatgcacgtgccacctggggagcggtgggggtagacagacacacaatgtcagcgtgccgtccttcagggaaacttttcatgctgagctcatggcagagtctcatgcagtggtcgggaatgactcagacacttccctgtacccgtcaccaagttccctgtaatgcagcatcttgcaaaacggtaatacgacctcacaggcagggacctgatcgtgacacagatgccatgtgaggtgttttgatgaaactcacacgctgggatcaacagcaccaataacaatttccagtttccttcattgtttatcttactttttcattttcttattatttttattttggagacagggtcttactgtcttgcccaggctggagtgcaatagtgtgatctcggctcactgcaacctctgcctcccggattcaagcgattcttctgcctcagcctcccgaggagctgagattacagtcgcgcaccaccatacccggctaatttctgtatttttgatagagacgggatttcactatgtcggtcaggctggtctagaactgctgaccacaagtgatccgtctgccttggcctcccaaagtgctgggattacaggtgtgagccacagcgcccggccttttttttcattggtttttacagtctattactgcagggtttcactttaccttgaatttcttttaactttaatttgcttttcattctttaacttcttttttttttttttttttgacacagagtttcattctggtcgcccaggctggagtgtaatagcgtgaccatggctcactgcaacctctacctgctgggttcaattgattctcctgtctcagcctcccaagcacctgggattacaggtgtccgccaccacgcccagctaatttttctgtttttactagagacggggtttcaccgtgttagacaggatggtctcgatctcctgacctcatgatccgcctgcctcggggttgggattacacactttgggaggccaaggcaggtggacgatcacaaggtcaggagttcgtgaccagcctgactaacacggtgaaaccccgtctctactaaaaatacaaaaatcagctgggcgtggtggcgggcgcctgtaatcccagttactcgggaggctgaggcaggagagtcgcttgaacccgggaggaggaggttgcagtgagcctagatcacgccattgcactccagcctgggcgacagagtgagactccgtctcaaaatgaatgaatgaatgaatgaatttcttataagaatttttttccccagacagtttgttttaagggataaattatccttctaagttagaagaaaataatgccagaagtctagacattcttatgctttggtcccgcttatcaaaccaaggttgctgaccttgataaccactcaagatccttacagtttataaagtcatttcctcaagttttctaagtggccgatcagagataaaccctagagaaatagttgatgtatgtttctagctttgggtgaccagcaaaatgtgatagaatattgccttttactggccgggtgcaacggctcacgtctgtaatcccagcactttgggaggctgaggcgggtggatcacttgaggtcaggagttcaaaaccagcttggccatcatggtgaaaccccgtctgtactaaaaatacaaaaaaattagctgggcgcggtgctgtgcacctgtaatccctgctactcaggaggctgaggcaggagaatcacttgaacccgggaggcggaggttgccgtgagccaagatcacgccattgcactccaacctgggtgacagagcgaggctccatctcaaaaaaaaaaaaaaaaaaagaatattgcctttaacatctttgtacaggtcatttatgaaatatcttgagctctgtgatggctaagagagaccttctttttctttctttcttttttttttttttgagacggagtttttttgtttttttgagatggagtctcgctctgttacccaggctggagtgcagtggcacgatctcagctcaccgcaacctccgcctcccgagttccagtgattctcctgcctcagcttcctgattagctgggattacaggcgcccgccaccacgcccaactaatttttgtatttttagtcgcgacggggtttcaccgtgttagccaggatggtcttgatcacttgacctggtgatccgcccaccttggcctcccaaagtgctgggatgacgggcgtgagccaccacgcccgtttgctttatgacttctaccagctcacagaagtctcctgtgtacatagaactccacttcccagccaggctcagtaactcacgtctgtgatcccagcactttgggaggctgaggcaggcagatcatgtgaggttgggagttcgagaccagcctggccaacatggtgaaaccccatctctagtaaaaatacaaaaattagccgagtgtggtggcaggcacctgttatcccagctgcacagcaggctgacacaggataatcgcttgaacccgggaggcggaggttgtagtgagccgagatcgcgccactgaattccagcctgggcgacagagtgagactccgtctcaaaacaaacaagcaaacaaaaatacccattacaatgttgttttaagattgttgtatatcaactgggcatggtggctcatgcttgtaatcccagcactttgagaggccgaggcggacagatcacgaggtcaggagatcgagaccatcctggccaacatggtgaaaccccatctctagtaaaaatacaaaaattagccgggcgtggtggtgggctcctgtaatcctagctactcgggaggctgaggcaggagaatcgcttgaacccgggaggtagaggttgcggtgagctgagatcgtgccactgcactccagcctgggtgacaagagcaaaactccgtctcaaaaaaataaaaaatttaaaaaaataaagaactcgacctcccaaaggtattggctaactccacgggcaaaaaaaccatacccattacaatgctgttttaagattgttgacctggtgcggtggctcatgcctgtaatcccagcactttgggaggctgaggcggacgaatcatgaggtcaggagatcgagaccatcctggctaacacggtgaaacgccatctctactaaaaatacaaaaaaaaaaaaattagccgggcgtggtggcgggcgcctgtagtcccagctactcgggaggctgaggcaggagagttgcttgaaccaggaggcggagcttgcagtgagctgagatcgtgccactgtagtccagccagggcgacaagagtgaaactccatgtcaaaaaatttaaaaaaattaaataaaagaactccacctcccaaaggtattggctaactccacgggcaaaaaaaaaaaaccataccccttacaatgccgttttaagattcttacgtatctcttcgaactccaacctgtcaccgttttagatccaaacccaccaatcacgaacctaaggatgaaagcaaaggctcagcagttgacctgggaccttaacagaaatgtgaccgatatcgagtgtgttaaagacgccgactattctatgccggtaaatcatactctctattgtttttttatttttattttatttatttatgtatttatgtatttatttattttttgagacggagtcttgctctgtcgcccaggctggactgcggtgacccgatctccgctctctgcaacctccacctcccaggtccatgccattctcctgcctcaacctcccgagtagctgggactacaggcgcccgccaccatgcccggctaatttttttgtatttttagtagagatggggtttcactgtgttagccaggagggtctcgatctcctgacctcgtgatctgcccgcctcggcctcccaaagtgctgggattacaggcgcgagtcaccgcgcatggcccagactctctaatgttgacgaacaagacgtttccgtcttctgcaggaatctcagaaccaatactgctcccatcgctggtgtcatgactacctggtttctgccccgaagtcaggtgtgggatttgaagtgactttggagggtctggttccccgtccgcgggacatctaagatggcacacactggacagggtggatgtgaaagtgaaatgaggctaagctatgactggtgcgaaacccaaccccacgctgggcgtggtggctcacgtctgtaatcccagcactttgggaggctgaggcgggcggatcatgaggtcaggagttcgagaccagtctggccaacacggtgaaacgctgtctctgctaaaaatacgaaagttagccgggcacagtggctcacaccagcactttgagaggctgaggcggatggatcacctggggccaggagttcgagactagtctgggcaacatggtgaaaccccgcctccactaaaaatataaaaattagctgggcatggtggtgggcgcctgtaatcccagttacttgggaggctgaggcaggagaatcgcttgaacccgggaggcagaggttgcattgagccgagactgtgccactgcactccagcctgggggacgagagcaagacttcatgtcaaaaaagaaaaagaaaaattagccggatgtggtggcacatgcctgtaataccagctattcaggaggctgaggcaggagaattgcttgaacctgggaggccgaggttgcactgagccgagattgtgccactgcactccagcctgggtgacagagtgagactccatctcaaaacaaaaacaaaaacaaaagcaaaaacaaaacaaaagtgtgtgctcaggaaacaaggtcctcatcacgaaatccttccaaatcccccatcttgtcatcacctgcgttctcagggtttgagaacagcgccagacctcatggggtggcccaggtgacactgtgagctatttacaagtcagtgtcttatgggaaaggagcacgtttccctgagaacctatttggtcccctccaagagctatgttcgttcaatacaattcaaatcacggcccttcatgcgtctgctcgggccaccattataaaatcctacccccagctccaaatacagtcccattgaactttgtgattttggagagtagagataaaacagtctagaatcccagagcgattttaccataccatggcaaactgactctcaactttagaaacacaaatgctgaaaaaaaaactaaggaaattttgaaaaagaaggtgaatgaaggagaacctgccttacctatatcaaaaggcactgaaaagttcatacccaatgtgcggattgctataagaatacacaagtaggccgggcgtggtggctcacgcctgtcatcccagcactttggaggccgaggcgggtggatcacgaggtcaggagatcgagaccctcctggctaacacggtgaaaccccgtctctactaaaaatataaaaattagccgggcgtggtggctggtgcctgtagtcccagctactcgggaggctgaggcaggagaatggcttgaacctgggaggcagaagcttgcagtgagccgagatcgcgccactgcactccagcctgggcgacagagcgagactctgtctcaaaaaaacaaaaacaaaagcaaacaaaacgaagaatatacaagtagattaatgaaatgtggccgggtgcggtggtgaggcaggagaattgcttgaacccgagaggtggaggttgcagtgagctgagatcgcaccactgaagtctagcctgggcaagcggagtgaggccctgtcttaacaaaaaaacaaagaaacaaaaaacaaacaaacaagaaaaaaacaaagcaaaacaaacagaaaagtatttcactaatatttactgctaagtgggattatttttattcaagcttttgtatctttagaaaaaaattgtggccgggcgcggtggctcacgcctgtcatcccagcactttgggaggccgaggagggtggatcacgaggtcaggagatcgagaccctcctggctaacacggtgaaaccccgtctctactaaaaatataaaaattagccgggcgtggtggcgggcgcctgtagtcccagctacttgggaggatgaggcaggagaatggcatgaacccgggagggagaggctacagtgagccgagatcgcgcccctgcactccagcctgggcgacagagcgagactccctctcaaaaaaaaaaaaaacagaaaaaagtcttggccgggcacggtggctcacgcctgtaatcccagcactttgggaggctgaggcaggcggatcacatgaggtcaggagttcgagaccagcctgaccaacatggtgaaaccctgtctctacccagaaaaatactttaaaaattagctgggcgtggtggcgggcacctgtaatcccaggtgctcgggaggttgaagcaggagaatggcttgaacccgggagggagaggctgcagtgagccgagatcacgccactgcactccagcgtgggcgacgagagcgaaactctgcctcaaaaaaaatctgaacatcattagcgtcaaattaagcatggtctgtcagcagccatcatagtcctatgtctctcttaggcagtgaacaatagctattgccagtttggagcaatttccttatgtgaagtgaccaactacaccgtccgagtggccaacccaccattctccacgtggatcctcttccctgagaacagtgagaaaaatgttcattgtttgtttattctctattccctccctccttccctctctccctccctctcgccttcgctgtgtcttttttcttttctttttctctttctttctttctttctttctttctttctttctttctttctttctttttctttctttctgtttctgtttctttcttcctttctttttctttctttctttccttctttcctctctttcttttctttctctttccctccctcccttctttcttttcttcacttccttccctccctccttctctccttttcctccctcctccttcccaccctacttcctctctctccttccttccctttcgttttcttttcctccctgcctcactcccttccttccttctctccctcctcttttccttccttcttccctccttccctttctctctctctctctttctctctttccccctcccctcccttcccctcctctcccctctcctttcctgacacggtcttgctctgttgcccaggctggagcgcagtggtgcaatcacagcttactgcaggcttcacctcctgagctcaaacaatccttctgcctcagcctcccacgtaggtggaactacacccatgtaccaccatgcccacctaattttttaaacacctttttttttttttagacagagtctcactctgtccaccaggctggagtgcactggcgcgatctcggctcactgcaacctctggctcccgggttcaagcgattctcctgcctcagcctcccgagtagctgggattataggcgcccaccgccacgcccagctaattcttgtatttttagtagagatggggtttcaccatgttggtcaggctggtctcaaactcctgacttcgtgatctgcccgcctcggcctcccaaagtgctgggagtacaggggtgagccaccgcgcccagccttaaatactttttgtagagatggggtgtcgctatgttacctgagctggtctcaaactcctaggctcaagtgatcctcccgcctcagtctcccaaagtgcagggattgtaagcatcagccaccacgcctggcctcttctttgttttttaattaattagaagtccatggtttatttttttgttttgttttgtttgtttttctgtgatggagtcttgttcttgtcgcccaggctggagtgcagtggtgcgatttcagcttaccgcaaactccgcctcccgggttcaagcgattctcctgcctcagcctcacaagtagctgggattacaggcacccgccaccaagcccggctaaattttgtattttaagtagagacggggcttcaccatgttggtctcgaactcctgaccttgtgatccacccgcctcagcctcccaaagtgctgggatgacaggcgggagccactgtgccgggcctcaatttaattatttttaagtttaatggatttccagggagttatgccgagtcggaaaaaaaagaccactccagagcgtgacacacacagtgatttcattaatacaactgtcttgaaatggcaaaattttacaaatagaaaacagtctcctgggttgcagggatataagcaggagtgagagctgagagaggtgggtttggctgtaaaagtgctccttggggaggccgggcgcagtggctcacgcctgtaatcccagcactttgggaggccgaggcgggcggatcacctgaggtcaggagttcgagaccagcctggccaacatggtgaaaccccgtctctactaaaaacacaaaaattagccgggcatggtggctcacgcctgtaatcccagcactttgggaggccgaggcaggcggatcacctgaggtcaggaattcgagaccagcctggctaacatggtgaaaccccatctctactaaaaatacaaaattagccaggggtggtggcacatgcctgtagtcccagctactcaggaggctgaggcaggagaatcacttgaacccgggaggcggagattgcggtgagctgagatcgtgccattgctctccagcctgggcaacaagagtgaaattccatctaaaaaaaaaaaaaaattaaaaagaaattagcggccgggcccagtggctcacacctgtaatcccagcactttgggaggccgaggtgggtggatcgcgaggtcaggagatcgagaccatcccggctaacatggtgaaaccccatctctactaaaaatacaaaattagccaggggtggtggcacatgcctgcagtcccagctactcaggaggctgaggcaggagaatcacttgaacctgggaggcggaggttgtggtgagccattgagctccagcctgggcaacaagagtgaaactctgtcttaaacacacacacagatgcacacacacgcacacacacacaaacaatgaaagaaaaacatctgtagtcccagcagtttgggaggctgaggcgggtggatcacttgaggtcaggaattcgcgaccagtctgggcaacatggtgaaaccccatctctactaaaaatacaaaaattagctgggtgtggtagtgggtgcctgttgtcccagatacttgggaggctgaggcaggagaattgcttgaacccgggaggtggaggttgcagtgagccgagatggcgccattgtactccagcctaggcgacggagcgagactctgtaaaaatgaataaataaatcattaaaagaatatgcaggatctctccatattattattattttttacaacggcttgtgtatctaccatggtctcaaaatacaaaaggcactctaaaaaggaattagagttttatttttttttttgagacggagtctcgctcttgtcacacaggctggagtgcagtgacgtggcctgggctcactgcaacctctgcctcctgggttcaagaaattctcctgcctcagcctccctagtagctgggattacagcggcccgacatcacgcccggctaatttttgtatttttagtagaggcagggtttcaccatgttagccaggccggtctcgaactccggacctcaggtgatcctcctgcctcggcctcccaaagtgctgggattacaggcgtgagccaccatgcctgggtttgtttttttatttttttgagacgaaggctcactctgtcgcccaggctggagtgcagtggcgtgatctcagctccatgcaacctctgcctcctgggttcaagaaattctcctgcctcagcctcccgagtagctgggattacagcggcccgacatcacacctggctaattttttatatttttagtagagactgggtttcaccatgttagccaggctggtctcgaactccggacctcgggtgatccacctgcctcggcctcccaaagtgctgggatcacaggcgcgtgccaccagacacagctaatttttgcatttttagtagagattgggtttctccatgttggccaggctggtctcgaactcctgacctcatgtgatccacccgcctcggcctcccaaaatgctggggtgacaggcgtgagccaccgcgcccggtcccgattcgagttctctttcatgtttgtgaacccaggtgggaagccttgggcaggtgcggagaatctgacctgctggattcatgacgtggatttcttgagctgcagctgggcggtaggcccgggggcccccgcggacgtccagtacgacctgtacttgaacgttgccaagtaggtgtgcccgtgggcagaggccgggctgtccctggtgcgggtgccatcggcgtggggtcgtcccccaaccttaccgcttaccgcagcaggcgtcaacagtacgagtgtcttcactacaaaacggatgctcagggaacacgtatcgggtgtcgtttcgatgacatctctcgactctccagcggttctcaaagttcccacatcctggtgcggggcaggagcgcagccttcggtatcccctgcacagataagtttgtcgtcttttcacagattggtgagtagcccgggacactccctcccaccctcagttctgtgataccacggctttagcgccaggccagatcccacgggaccacgtggctcccaacgcagacgttggcctctcacatttccagaggctggacgttggaggtcagcgtgctggctggctgggctcctcgggaggtctcttcctggcttggagagagggtcatcttctcactgtgtcttcacgtggtgtagagagagagagagatgaagttctggggtctcttgttagaagggcactaacaccatcatgggtcccatcatgggtcataggatccctcaccatgggtcatgggaccccccatcatgggttccatcatgggtcccatcatgggtcatagaatcccccatgatgggtttcatcatgagtcatagaaccccctatcatggattccatcatgggtcatgggaacccccatcatgggtcccatcatggttcataggatcccccatcatgggtttcatcatgagtcatagaaccccctatcatggattccatcatgggtcatgggaacccccatcatgggtcccatcattggtcataggaccccctatcatgggttccatcatgggtcatgggacaccccccatcatgggttccaccatgagtcatgggatccctcatcgtgggttccatcatgggtcatgggaccccccatcatgggttccatcatgggtaataggacccccccatcatgggttccaccatgagtcatgggatcccttatcatggattccatcatgggtcatgggaccccctatcatggattccatcatgggtcatgggacccccccatcatgggtcccatcatgagtcatgggatccctcatcatgggtttcatcatgggtcataggacccccatcatggattccatcatgggtcatgggaacctccatcatgggtcccattatgggtcatagaattccccatcatgggttccatcatgagtcatgggaccccccatcatgggttccacatgggatccctcatcattggttccatcatgggtcataggatcccccatcataggtcccatcatgggttccacatgggatcccccatcataggtcccatcatgggtcatgggaccccccccatcatgggttccatcacgggtcatgggaccccccccatcatgggttccatcacgggtcatgggaccccccccaatcatgggttccatcatgggtcatgggaacccccatcatgggtttcatcatgggtcataggatcccctatcatgggttccatcatgggtcatgggaccccccccatcacgggttccatcatgggtcatgggaacccccatcatgggtttcatcatgggtcataggatcccctatcatggattccatcatgagtcatgggatccctcatcatgggttccatcatgggtcatgggacccccccccccatcatgggtcgtgggacccccacccccatcatgggtcatgggagcccccattgtgagtcatggaatccctcatcatgggtctcatcatgggccatgggtcccaccatgggtcatgggagcccccatcatgagtcatggaatccctcatcatgggtcatgggtcccatcatgggtcatgggagcccccatcatgggtcatgggtcccatcatgggtcacgggagcccccatcatgggggtccaccctcataacttcagcccacgccagtcacctcccaaagaccccaccttctaacaccgcccaaccaggggttagagcttcagtggaggaatttgggacagaaggacacacactttcagtccacgatacccgagctccgaggaacctcccaggtggtgagaatgtcaatatgcccagagctgacgtgccctgaacccaagggcagggtgctcggatgcttcagaagaggagggggaaacgaggaagaggaggagaaggacaaggagaagagaagggaggagaggaagaacatgagcagggggaggaggaggagaaggaaaagaaacagaggaaaaggagggggaggaggtagagatggagagggaaggaagaggaggaagagaagaaaacggagaaagaggagggggaggaggaggaggaagagaaaatggagaaagaggagggggaggaggtggagatggggaggggaggaagaggaggaagagaagaaaacaggagggggaggaagagaagaaaatggagggaaaggagggggaggaggtggagatggggagagaaggagcaggaggaggagaagaaagaagaggaagaaaggaggaggagagggtggaggaggaagagggtgaaggagagagggaggagaatggagaggaggaggaggcgggggagggaagagaaggaggtgaggaggaggaaggggaggatagagaaggtggggtagaagggtggtgaggtggggagggagaaggagggggaggaggagagaggaggactggggggaggaagggggaggaggggagagaggaggagggaggagggagaaggagggggagaggatggaggaaggggagcagatggaggaaggcgagcaggagggggaggaaaaaggaggggcaggaggaagaaggagcgggaggagaggaggaggaggaggaggagaatgggcagggaggaggagagggaagaaaaggagggggaggaggaaggagggggaggagggtggagggggaggagggtggagggggaggagggtggagggggaggaggaaggagggagaaggaggggagaggatggaggaagaggagcaggagggggaggaaaaaggaggaggggcaggaggggaggaggagaaaggagagtggaggaggagggtgacaaggagaatggggaggagagaggagtggtagaaaaaggagagagaggaagagtaggagcggagggggaggaggggaggggaggggaggggagaaagaccaggagggtaggaggagggggaggaggaggagggggccagggctggacttccttccaggggcccaggggagacccagccaggcagccggacacacagggctgggctggtagtcgggtcacacctgaggatgtggaagtgcctggggttcccgggaagtggaggatgtcctgggccactgggagaggcggcacatcccctggggggcggaggtgggggaggtgagaagccaggcaggggccaggaagtggaagcttcaggaggatgtgtgcaggtcaaagtgtaggagtctgcatcttgaatgatgcagagagggcctccaaggctccagtgtgctgcaggtgggcagagggggcatgggaagtaggggttggccctgggcaggggtggggagtgggccaggctgcccagcggggctgagcctagagatggaaggggcagggacaaagatgtgcagctgccagtccttggaaaagctgaacgcctggtacttcagagaacaaaagggtggttctgaaggctgctcccaggacagtggggctccgagggtgcaaccccaaggctcactcctcccagtgcccccaacgcggctcagtcctgtgtctctgcctgtagagtttctttgttccctcttccttcctggtgtttttctctcccgctctccaaatgcataggagaagtaatttgaagtatctccagaaaaaaaaagagaaaaagaaaaagaattgatttcttgtactcctaaatcctaaaagtgtttttctcgttgctagagatattaactccacccaacatgactgcaaagtgtaataagacacattcctttatgcactggaaaatgagaagtcatttcaatcgcaaatttcgctatgagcttcagatacaaaaggtaaactttcaccccgcccccagcccccccacccccgtggacatcccttatttttggtaagtcgcactctggggccttgaaacgggcaacaatctcctctgataacgtcacagaaggcatggatcattaaaaaacaaaaacaaaaacaaaaggccgggcgctgtggctcacgcctgtcatcccagcactttgggaggctgaggcaggtggatcacaaggtcaggagatcgagaccatcctggctaacgcggtgaaaccccgtctctactaaaaatacgaaaaaaaattagctgggcgtggtggcacggtcttgtagttccagctactggggaggccgaggcaggagaattgcttgaacccaggaggtgggggttgcagtgagccgagatcgcgccattgcactccagcctgggcgacagagcgagactccgtctcaaaaaaaaaaaaaaagtttgagactgtatgtggtctgttgttttttatttttattatttctattgttatgtggttttttaattttttcttgaactttcttttttcttttgtagtgatctacagattcaatgcaagcttcccagatatttttgatccacagttcgttggatcagttgaacataaatattgatttatttattgagccactctggctctgtagcccagactggagtgcagtggcttaaatcttgactcactgcaacctctgcctcccgggttcaagtgattctcctgcctcagcctcccgagtagctgggattatgggcacccatcaccacacccagctaagtttttgtatttttagtagaaacagagtttcatcatgttggccaggctggtctcaaactcctgacctcaggtgatccacccgcctcggcctcccaaagtgctgggattacaggtgtgagccttcgtgcgcagcctatgtgttatatttagtttgtattttattttattgtattttattatttatttatttatttttgagacggagtcctgctctgtcacccaagctgtttgtttgtttgtttgtttgtttattatttttgagacggagtcctgctctgttgcccaggctggggtgcagtggcatgatctcagctcactgtaacctccgcctctcgggttcaaccaattctcagcttcagcctcctgagtagctggaattacaggcacctgccaccatgcctggctaatttttttttgtatttttagtagagatggggtttcaccatcttgtcctggctggtcttgaactcctgacctcatgatccacccgcctcagcttcccaaagtgctgggattacaggcgtgaggcactgtgccccgccatatatttattatttatgctcaaatactaattatttcatatgcaatttttctgtaagtctaaatctgctaaaaaacgttaggtctattaatttcttttatattaccaagtgttttttagccaatctgtttgggttttttttttttttaagaaaataaatggctgggtgcagtagcttacgcctgtaatcccagcactttgggaggccgaggcaggacgatcacgaggtcaggagatagagaccatcctggctaacatggtaaaaccccgtctctactaaaaatacaaaaaaaaattagctgggcgtggtggcgggcgcctgtagtcccagctacttgggaggctgaggcaggagaatggcgtaaacccagaaggcggagcttgcagcgagctgagattgtgccactgcactccatcctgggcaacagagtgagactccgtctcaggagaaaaaaaaaaaaagaaaataaatacagcacagacttctttctttcactgatttgagggagcaggcatagctgcagccacaggcagagtcgtagctagtctgatgttgcaccccctacctagttcgctggcctggcataggctgtctgtggctacccctgagtgcatctggacacagtctgggaggacggtgggtcttgtttgtccaccggcctcacaaagccccctcccaccaaggtcttgccacagtggcccataagaaaccttttgggccaggcgcgatggggcatgcctgtaatcccagcactttgggaggccgaggtgggcagatcatgaggtcaagagatcaagaccatcctggccaacatggtgaaactttgtttccactaaaaatacaaaaaattagccaggcgtggtggcacgcacctgtagtcccagctactcgggagactgaggcaaaagaatcacttgaatccgggaggcggaggttgcagtgagccgagatcacgccactgcattccagcctgggtgacggagtgagactccgtctcataaataaataaacaaacaaaccttttggtcaggtgctatttactcctaagctcattattttgcccccactgctgcccgaaggccttcccagagccctcactgttttgctggttttcctggagggagaaatttgagtttgggaggaggaggctttcagggacggtccagacactcaaaagtttgcttgcttttgtgttgcagagaatgcagcctgtaatcacagaacaggtgagtgttccctacccccagccgctgtacttgacattgcaaagggtgagttttattattattaagaataaaatgataaaaaatattaataattcttattaataaaataatgaaaatattattaataataaatgttattattcaatgttcagtgactttcattggacagactcttgagtgtcacccttactgcgatcttgcaaaattgggatatttcacatccccaaattgagggatgggaaaaggaagagtcagggatgacacctcccaaggtgtgagagccagatgctatggctggccaggtgctgtccaaacgaggtccacccatttgccccagattccttaccctgggccaggcagccccagtccagcaggaacaagctaccaaaccatagctccacccagcagagacaagcattcagacaggtggcccagacctcagacagaggaccctcccccagcccctgcctgggatctgcaggaacagacagccggaccacagcattctctgtcccctctctctctgtccatctctttccccgtatctctttctctgtctctgaatctctctgcctttctccctccctctctttctctctctctcgtctctatctccctccccctctctgtatctccctccctttttccctccctctatctttctggctctctccctgtgtctatctctccctgtccccatctctctctgtgtctctttccctccctctctcattctccttccctctctctatctttctctccctgtctctatctcccgctccgtgtctgtctctatctttctccctttctccctccatctctctctccctgtctctctctccccgttcccatctctctctctgtgtctctgtctctccctctctcattctccctccctcttttattctccctccctctctttctctctctctctctctctcctggtctctgtctcccccctccccatctctttctctgtgtctgtctctgtaaccttctccatttctccctccatctctctgtctctccctgtgtctatctcccactgtccctgtctgtttctccgtgtctgtctctgtatctctctccctttctccctccatctttctctctctcccccggtctctatcttcccttctccccatctctctctctctgtctgtctctgtatctctctccctttctccctccatctttctctctctctcccggtctctatctccccctctccccgtctctttctccgtgtctgtctctgtgtctctctccctttctccctccctccatctttctctctctctcccggtctctatttcctccctccccatctctttctctgtgtctgtctctgtatctctctccctttctccctccatctttctctctctctcctggtctctatctccccctctccctgtctctctgtgtctgtctctgtatctctctccctttctccctccctctctttatctctctctctctctctgcctctatctcccactgtccaaatctgtgtctctgtctctccctctttcattctccctctctttctctatctctctccttgtctatatctccccctctcccagtctctctgtatctctgtatctctctccctttctccttccctctcttcatctttctttttgaaacggagtttttctcttgttgcccaggctggagtgcgatggcacgatctcggctcactgcaacctccgcctcccaggttcaagcgaatctactccctcagcctcccaagtagctgggattacaggcactcgccaccatgcccaactaacttttttttttttttgtatttttagtagagacagggtttcactagtgggccaggctggtctcgaactcctgacctcaggcgatccacctgcctcatcctctcaaagtgctggaattacaggtgtgagccaccgtgcccggcccctctctttatctttctagctctctccctgtctctctctcccttcccctctctgtctctctctcccttcccctctctgtctctctctcccttcccctctctgtctctctctcccttcccctctgtctctctctcccttcccctctctgtctctctctcccttcccctctctgtctctctctcccttcccctctgtctctctctcccttcccctctctgtctctctctcccttcccctctctgtctctctctcccttcccctctctgtctctctctcccttcccctctctgtctctctctcccttcccctctctgtctctctctccctttccttctctgtctctctctcccttcccctctctctctctccattcccctgtctctatgtctctccctttctctctcccattatttctctgtgattgtctctttctttctctgcctctgtctgtctgtccccctgtattagtccattgtcacactgctgataaacatatacccaagactgggtaatttataaacaaaagagggttcctggactcacagttccatgtggctggggaggcctcacgatcacggcagaaggtgaaggaggaggaaaggcatgtcttacatgtcagcgggcaagacagaatgagacagtcgccgggcgcagtgactcatgcctgtaatcacagcaccttgggaggacgaggtgggtggaccacgaggtcaggagttcaagaccagcctggccaacatggtgaaacctagctctactaaaaataaaaaaaattacccagacgtggtggtggcaggcgcctgtggtcccagctactcgggaggctgaagcaggagaatcgcttgaacccgggaggcagaggttgcagtgagttgagatcgcgccactgcactccagcctgggtgacagagtgagactccgtctcgagaaaaaaaaaaaaaaaaaaaaaatgagagccgagagaaaggtttcccttattaaaccattagatctcgtgagacttattcactaccccgagaacagtgtggggggaaactgcccccatgattcagttatgtcctacccagtccctctcacaacacgtgggagttataagggctacaattcaagatgagatttgggtggggacacagccaaactacatcacccactctctctgtctctctgcttctgttttcctctctgtctctgtttttctttccctctctctgtctctttgtatctctgtctctctctctctgtctccgtttctatctctgtctctctctgtccatcacccactctgtctctttgtctctctatgtctctctgtttctgtttttctgtttttctctttccctctctctttgtatctatgtctttctgtctctccgtttctgtctctctctctgtccatcacccactctgtctgtctttgtctatgtctctctgtttttctgtctctttttctctttccctctctgtctcccctctctgtgtctctttgtatctccgtctctccctgtctgtttctatctctgtctctctctgtccatcacctactctgtctctttgtctctgtctgtttctgtttttctttctttttccctctgtctcccttctctgtgtctctttgtatctctgtctctctctgtttctatctgtctctctctgtccatcacccactatctctgtctatgtctgtttttctgtctctgtttttctctctccctctctctgtctcccctctctgtgtctctttgtatctctttctctctctctgtctctccctgtctgtttctatctccgtcctcctgtattagtccattttcatggactaatgaggatccttcccgcttctcccagctcctggggactctggggtccctgggcttgtggccatatcactccactctctgcctccgtctccacatggccttctcctctgcatccgtgtctcctttttatttatttatttatttattttgagacagagtctcgctctgtcacccaggctggagtgcagtggtgcgatctcagctcactgcaacctccgcctcccaggttcaagcgattcccctgcctcagcctcccgagtagctgggactacaggtgcacaccaccacgcctggctaatttttgtattattagtaaagatgggattttaccttgttgctcaggttgacctcgaactcctgacctcaggtgatctacccacctcagcctcccaaagtgctgggattacaggtgtcagccaccgcgcccggccacgtctcctcttcttataaggatattggtcattgcatttagggccccccctgatccaaaatgacatcatctcaatctatatcttaacgacatctgccatgaacaggtatttcatcataaggccccattgtaaggtgctgggggctaagactgcaacatatgaattttgggggatcacaatgcagcctcattcttttgtacccatcaaatatgaatggtcttttcttcttttttttttttttttgagacggagtctcgctctgtcacccaggctggagtgcagtggcgcaatctcggctcactgcaagctccgcctcccgggttcccgccactctcctgcctcagcctcctgagtagctgggacctcaggcgcccgccaccgggcccggctaattttttgtatttttagtagagacggggtttcactgtggtctcgatctcctgaccttgtgatctgcccgccttggccctccaaagtgctgggattacaggcgtgagccaccgcgcctggccgagctatttcatcataaggccccactgtaaggagctgggggctaagactgcaacatatgaattttgggggatcacaatgcagactcattcttttgtacccatcaaatatgaatggtcttttcttctacttttttgtttggtttttaatttcagacaggctctcactctgtcacccacgctagagtgcagtggcacagtcacggctcactgcaatctctgcctcctgggctcaagggatcctcctgcctcagccccccaagtagctgaaactacaggcgtttgccaccaggtcagctcatttaaaaaaaaatttggctgggcgcggtggctcacgctggtaatcccagccctttgggaggccgaggcgggtggatcatgaggtcaggaaatcaagaccatcctggctaacatggtgaaaccctgtctctactaaaaaaaaaaatacaaaaaattagctgggcatggtagcgggtgcctgtagtcccagctactcgggaggctgaggcaggagaatggcatgaacccgggaagcggagcttgcagtgagccgagatcacgccactgcactccagcctgggggacagagtgagactccatctcaaaaaaaaaaaaatttttttttggctggacgtggtggctcacactggtaatcccagcactttgggaggctgaggcgggtggatcaccggaggttgggagttcgagaccagcctgaccaacatggagaaaccccgactctactaaaaatacaaaattaaccgagcgtggtggtgggcgcctgtaatcccagctactcgggaggctgaggcaggagaatcgcttgaatctgggaggcggaggttgcggtgagccgagttggtgccactgcactccagcctgggcaacaagagtgaaactctgtctcaaaacaaaacaaaacaaattttcttctagagatggggtcttggtttgttgcccaggctggtctcaaactcctaagctcgaggtatccccccacctcagcctccctggtagctgggaccaaagacatgtgtcaccacgtctggtaatttttacagttttttttttttggtaaagatggggtcttgctatgttgcccaggctggtgtcaaactcctgggcttaaggaattctcccacctcagcctccaaaagtgctgtggtgagaggcataaaccgtagcacccagcccctcttttcttcttttatttatttatttatttatttatttatttatttagagacagagtctcactctgttgcccaggctgcagtgcagtggtgccatctcgcctcactgcaacctccacctctcaggtttaagctattctcctgcctcagcctcctgagtagctgggattacaggtgcctgccaccatgccaggctaatttttgtatttttagtagagacagggtttcaccatgttggccaggctggtctcgaactcctgacctcaggtgatccacctgccttggcctccctaagtgcccaggtgagagtcatgagccaccatacctggcccctcttctttattttctttcaaaccacaggtcagagacagaacctccttccagctactcaatcctggaacgtacacagtacaaataagagcccgggaaagagtgtatgaattcttgagcgcctggagcaccccccagcgcttcggtgagtgggctgtgcggggtgcgcggggtgagcggggtgagcggggtgcgcggggtgagcggggtgcgcggggtgagcggggtgcgcggggtgagcggggtgagcggggtgagccgggtgcgcggggtgagccggctgcgcggggtgagcggggtgagcggggtgagcggggtgagcggggtgcgcggggtgagcggggtgagcggggtgcgcggggtgagcggggtgagccgggtgcgcggggtgagccgggtgagcggggtgcgcggggtgagcggggtgagcggggtgcgcggggtgagccgggtgcgcggggtgagcggggtgagccgggtgcgcggggtgagcggggtgagcggggtgagccgggtgagccgggtgagccgggtgcgcggggtgagccgggtgcgcggggtgagcggggtgcgcggggtgagcggggtgcgcggggtgagccgggtgcgcggggtgagcggggtgcgcggggtgagcggggtgcgcggggtgagccgggtgcgcggggtgagcggggtgagcggggtgagccgggtgccccgggtgagcggggtgcgcggggtgagccgggtgcgcggggtgagcggggtgagcggggtgcgcggggtgagccgggtgcgcggggtgagccgggtgcgcggggtgagccgggtgagcggggtgagcggggtgcggggtgcgcggtgcgcggggtgagcggggtgcgcggggtgagccgggtgcgcggggtgagcggggtgagccgggtgccccgggtgagcggggtgcgcggggtgagccgggtgcgcggggtgagcggggtgcgcggggtgagcggggtgcgcggggtgcgcggcgtgagccgggtgcgcggggtgcgcggggtgagcggggtgcgcggggtgcgcggggtgcgcggggtgagcggggtgcgcggggtgcgcggcgtgagccgggtgcgcggggtgagcggggtgcgcggggtgagcggggtgcgcggggtgagccgggtgcgcggggtgagccgggtgagccgggtgcgcggggtgagcggggtgcgcggggtgagccgggtgagccgggtgcgcggggtgagccgggtgcgcggggtgagcggggtgcgcggggtgagccgggtgcgcggggtgagcggggtgcgcggggtgagcggggtgcgcggggtgagcggggtgcgcggggtgagccgggtgcgcggggtgagcggggtgagcggggtgagccgggtgccccgggtgagcggggtgcgcggggtgagcccggtgagccgggtgcgcggggtgagccgggtgcgcggggtgagccgggtgagcggggtgagcggggtgcgcggtgcgcggggtgagcggggtgcgcggggtgagccgggtgcgcggggtgagcggggtgagccgggtgccccgggtgagcggggtgcgcggggtgagccgggtgcgcggggtgagcggggtgcgcggagtgagcggggtgcgcggggtgcgcggcgtgagccgggtgcgcggggtgcgcggggtgagcggggtgcgcggggtgcgcggggtgcgcggggtgagcggggtgcgcggggtgcgcggcgtgagccgggtgcgcggggtgagcggggtgcgcggggtgagcggggtgcgcggggtgagccgggtgcgcggggtgagcggggtgcgcggggtgagccgggtgcgcggggtgcgcggggtgcgcggggtgcgccgggtgcgcggggtgagccgggtgcgcggggtgagcggggtgcgcggggtgcgcggggtgcgcggggtgagccgggtgcgcggggtgagcggggtgagccgggtgcgcggggtgagcggggtgagccgggtgcgcggggtgagcggggtgcgcggggtgagccgggtgcgcggggtgagcggggtgcgcggggtgagccgggtgcgcggggtgagcggggtgcgccgggtgagcggggtgagcggggtgagccgggtgcgcggggtgcgcggggtgcgccgggtgcgcggggtgagccgggtgcgcggggtgagcggggtgcgcggggtgcgcggggtgcgcggggtgagccgggtgcgcggggtgagcggggtgagccgggtgagcggggtgcgcggggtgagccgggtgcgcggggtgagcggggtgcgcggggtgagccgggtgcgcggggtgcgcggggtgagccgggtgcgcggggtgagcggggtgcgcggggtgagcggggtgcgcggggtgagccgggtgcgcggggtgagccgggtgcgcggggtgcgcggggtgcgcggcgtgagccgggtgcgcggggtgcgccgggtgcgcggggtgagcggggtgagccgggtgcgcggggtgagcggggtgcgcggggtgagcggggtgcgccgggtgagccgggtgcgcggggtgagccgggtgcgcggggtgagcggggtgcgcggggtgagccgggtgcgcggggtgagcggggtgcgccgggtgagcggggtgcgcggggtgagcggggtgcgccgggtgagcggggtgcgcgggctgagcggggtgcgccgggtgagcggggtgcgcggggtgagcggggtgcgccatcctgggtcacggaaacactcctctcctgcaaaggagaggagattcactcccccagtttctgtgaccccaaaaaggaccctgaacccgacggtgaactcacagcttgctcttactcacgagaggagacgtggaggggaaacaaggtcgtcccactgacagacaccccctgggccttgtaataaagaccgaggcgggcggatcacaaggtcaggagatcgagaccatcccggctaacacggtgaaacccggtctctacaaaaaatgtaacaaattagccaggcgtggtgatgggcgcctgtagtcccagctactcgggaggctgaggcaggagaatggcgtgaacccgggaggcggaggttgcagtgagccgagatcgcgccactgtgctccatccagcctgggcgacaagagcgagactccatctcaacaacaaaacaaaaggatcgcctcagagtagaacttctggccgggcacggtgactcatgcctgccatcccagcactttgggaggctgaggtgggtagatcacctgaggtcaggagttcgagaccagcctgaccaacatggaaaaaccctgtctctactaaaaatagaaaaattaggtcaggtgctgtggcttaagcctgtaacctcagctaccagggaggctgaggcaggagaatcgcttgaattgtgttccctcaaaattcgtgtgttgaagctttgatcccccaggacctcagaatgtgactgtgtttggagttggggtgtttaaagaggcgattaaggtaaaatgaggtcattagggtgggccctaatctaacaggactggggtccttataagaagaggagatgaggacacagacacacacagagggacgaccctgtgggacacagggagaagacggcgtctccaagcccaggagaggggcctcaggaggaaccagcactgcccacacctggatctcagacctccagcctccagggctgtgggagaatcaatgtgttttgtttctaagccgcccagcctctggtattctgtgacagcagcctgagatggactaagccatctcataagaagagacgaggacacagacacacacagagggacgaccctgtgagggcacagggagaagacggcgtctccaagcccaggagaggggcctcaggaggaaccagcactgcccacacctggatctcagacctccagcctccagggctgtgggagaatcaatgtgttttgtttctaagccgcccagcctctggtattctgtgacagcagcctgagatggactaagacatctcataagaagaggagatgaggacacagacacacacagagggacgaccctgtgagggcacagggagaagacggcgtctccaagcccaggagaggggcctcaggaggaaccagccctgcccacacctggatctcagacctccagcctccagggctgtgggagaatcaatgtgttttgtttctaagccgcccagcctctggtattctgtgacagcagcctgagatggactaagacatctcataagaagaggagatgaggacacagacacacacagagggacgaccctgtgagggcacagggagaagacggcgtctccaagcccaggagaggggcctcaggaggaaccagcactgcccacacctggatctcagacctccagcctccagggctgtgggagaatcaatgtgttttgtttctaagccgcccagcctctggtattctgtgacagcagcctgagatggactaagccatctcataagaagagacgaggacacagacacacacagagggatgaccctgtgagggcacagggagaagacggcgtctccaagcccaggagaggggcctcaggaggaaccagcactgcccacacctggatctcagacctccagcctccagggctgtgggagaatcaatgtgttttgtttctaagccgcccagcctctggtattctgtgacagcagcctgagatggactaagacatctcataagaagaggagatgaggacacagacacacacagagggatgaccctgtgagggcacagggagaagacggcgtctccaagcccaggagaggggcctcaggaggaaccagccctgcccacacctggatctcagacctccagcctccagggctgtgggagaatcaatgtgttttgtttctaagccgcccagcctctggtattctgtgacagcagcctgagatggactaagccatctcataagaagagacgaggacacagacacacacagagggacgaccctgtgaggacacagggagaagacggcgtctccaagcccaggagaggggcctcaggaggaaccagcactgcccacacctggatctcagacctccagcctccagggctgtgggagaatcagtgttttgtttctaagccgcccagcctctggtattctgtgacagcagcctgagatggactaagccatctcataagaagagacgaggacacagacacacacagagggacgaccctgtgagggcacagggagaagacggcgtctccaagcccaggagaggggcctcaggaggaaccagccctgcccacacctggatctcagacctccagcctccagggctgtgggagaatcaatgtgttttgtttctaagccgcccagcctctggtattctgtgacagcagcctgagatggagtaagacatctcataagaagaggagatgaggacacagacacacacagagggacgaccctgtgggacacagggagaagacggggtctccaagcccaggagaggggcctcaggaggaaccagccctgcccacacctggatctcagacctccagcctccagggctgtgggagaatcaatgtgttttgtttctaagccgcccagcctctggtattctgtgacagcagcctgagatggactaagccatctcataagaagagacgaggacacagacacacagagagggacgaccctgtgaggacacagggagaagacggcatctccaagcccaggagaggggcctcaggaggaaccagcactgcccacacctggatctcagacctccagcctccagggctgtgggagaatcaatgtgttttgtttctaagccgcccagcctctggtattctgtgacagcagcctgagatggactaagacatctcataagaagaggagatgaggacacagacacacacagagggacgaccctgtgaggacacagggagaagacggcgtctccaagcccaggagaggggcctcaggaggaaccagccctgcccacacctggatctcagacctccagcctccagggctgtgggagaatcaatgtgttttgtttctaagccgcccagcctctggtattctgtgacagcagcctgagatggactaagccatctcataagaagagacgaggacacagacacacacagagggacgaccctgtgagggcacagggagaagacggcgtctccaagcccaggagaggggcctcaggaggaaccagccctgcccacacctggatctcagacctccagcctccagggctgtgggagaatcaatgtgttttgtttctaagccgcccagcctctggtattctgtgacagcagcctgagatggactaagccatctcataagaagagacgaggacacagacacacacagagggacgaccctgtgagggcacagggagaagacggcgtctccaagcccaggagaggggcctcaggaggaaccagccctgcccacacctggatctcagaactccagcctccagggctgtgggagaatcaatgtgttttgtttctaagccgcccagcctctggtattctgtgacagcagcctgagatggactaagacatctcataagaagaggacatgaggacacagacacacacagagggacgaccctgtgggacacagggagaagacggggtctccaagcccaggagaggggcctcaggaggaaccagccctgcccacacctggatctcagacctccagcctccagggctgtgggagaatcaatgtgttttgtttctaagccgcccagcctctggtattctgtgacagcagcctgagatggactaagacatctcataagaagaggagatgaggacacagacacacacagagggacgaccctgtgggacacagggagaagacggggtctccaagcccaggagaggggcctcaggaggaaccagccctgcccacacctggatctcagaactccagcctccagggctgtgggagaatcaatgtgttttgtttctaagccgcccagcctctggtattctgtgacagcagcctgagatggactaagacatctcataagaagaggagatgaggacacagacacacacagagggacgaccctgtgaggacacagggagaagacggcgtctccaagcccaggagaggggcctcaggaggaaccagccctgcccacacctggatctcagacctccagcctccagggctgtgggagaatcaatgtgttttgtttctaagccgcccagcctctggtattctgtgacagcagcctgagatggactaagccatctcataagaagagacgaggacacagacacacacagagggacgaccctgtgagggcacagggagaagacggcgtctccaagcccaggagaggggcctcaggaggaaccagccctgcccacacctggatctcagacctccagcctccagggctgtgggagaatcaatgtgttttgtttctaagccgcccagcctctggtattctgtgacagcagcctgagatggactaagccatctcataagaagagacgaggacacagacacacacagagggacgaccctgtgagggcacagggagaagacggcgtctccaagcccaggagaggggcctcaggaggaaccagccctgcccacacctggatctcagaactccagcctccagggctgtgggagaatcaatgtgttttgtttctaagccgcccagcctctggtattctgtgacagcagcctgagatggactaagacatctcataagaagaggacatgaggacacagacacacacagagggacgaccctgtgggacacagggagaagacggggtctccaagcccaggagaggggcctcaggaggaaccagccctgcccacacctggatctcagacctccagcctccagggctgtgggagaatcaatgtgttttgtttctaagccgcccagcctctggtattctgtgacagcagcctgagatggactaagacatctcataagaagaggagatgaggacacagacacacacagagggacgaccctgtgggacacagggagaagacggggtctccaagcccaggagaggggcctcaggaggaaccagccctgcccacacctggatctcagaactccagcctccagggctgtgggagaatcaatgtgttttgtttctaagccgcccagcctctggtattctgtgacagcagcctgagatggactaagacatctcataagaagaggacatgaggacacagacacacacagagggacgaccctgtgggacacagaagacggggtctccaagcccaggagaggggcctcaggaggaaccagccctgcccacacctggatctcagacctccagcctccagggctgtgggagaatcaat

SEQ ID NO: 14 - DNA, 1541 bp, Homo sapiens
  source 1..1541: mol_type = genomic DNA
cttcggtttctcttcggggaaagctgctttcagcgcacacgggaagatatcagaaacatcctaggatcaggacaccccagatcttctcaactggaaccacgaaggctgtttcttccacacagtactttgatctccatttaagcaggcacctctgtcctgcgttccggagctgcgttcccgatggtcctcctttggctcacgctgctcctgatcgccctgccctgtctcctgcaaacgaaggaagatccaaacccaccaatcacgaacctaaggatgaaagcaaaggctcagcagttgacctgggaccttaacagaaatgtgaccgatatcgagtgtgttaaagacgccgactattctatgccggcagtgaacaatagctattgccagtttggagcaatttccttatgtgaagtgaccaactacaccgtccgagtggccaacccaccattctccacgtggatcctcttccctgagaacagtgggaagccttgggcaggtgcggagaatctgacctgctggattcatgacgtggatttcttgagctgcagctgggcggtaggcccgggggcccccgcggacgtccagtacgacctgtacttgaacgttgccaacaggcgtcaacagtacgagtgtcttcactacaaaacggatgctcagggaacacgtatcgggtgtcgtttcgatgacatctctcgactctccagcggttctcaaagttcccacatcctggtgcggggcaggagcgcagccttcggtatcccctgcacagataagtttgtcgtcttttcacagattgagatattaactccacccaacatgactgcaaagtgtaataagacacattcctttatgcactggaaaatgagaagtcatttcaatcgcaaatttcgctatgagcttcagatacaaaagagaatgcagcctgtaatcacagaacaggtcagagacagaacctccttccagctactcaatcctggaacgtacacagtacaaataagagcccgggaaagagtgtatgaattcttgagcgcctggagcaccccccagcgcttcgagtgcgaccaggaggagggcgcaaacacacgtgcctggcggacgtcgctgctgatcgcgctggggacgctgctggccctggtctgtgtcttcgtgatctgcagaaggtatctggtgatgcagagactctttccccgcatccctcacatgaaagaccccatcggtgacagcttccaaaacgacaagctggtggtctgggaggcgggcaaagccggcctggaggagtgtctggtgactgaagtacaggtcgtgcagaaaacttgagactggggttcagggcttgtgggggtctgcctcaatctccctggccgggccaggcgcctgcacagactggctgctggacctgcgcacgcagcccaggaatggacattcctaacgggtggtgggcatgggagatgcctgtgtaatttcgtccgaagctgccaggaagaagaacagaactttgtgtgtttatttcatgataaagtgatttttttttttttaaccca

SEQ ID NO: 15 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 16 - DNA, 20 bp, Homo sapiens
  source 1..20: mol_type = genomic DNA
tgagttcccaacttcattag

SEQ ID NO: 17 - DNA, 20 bp, Homo sapiens
  source 1..20: mol_type = genomic DNA
ctaatgaagttgggaactca

SEQ ID NO: 18 - RNA, 20 nt, Homo sapiens
  source 1..20: mol_type = genomic RNA
tgagttcccaacttcattag

SEQ ID NO: 19 - DNA, 20 bp, Homo sapiens
  source 1..20: mol_type = genomic DNA
tcagaccgtaccttgcaaca

SEQ ID NO: 20 - DNA, 20 bp, Homo sapiens
  source 1..20: mol_type = genomic DNA
tgttgcaaggtacggtctga

SEQ ID NO: 21 - RNA, 20 nt, Homo sapiens
  source 1..20: mol_type = genomic RNA
tcagaccgtaccttgcaaca

SEQ ID NO: 22 - DNA, 20 bp, Homo sapiens
  source 1..20: mol_type = genomic DNA
gtaccttgcaacaaggtaat

SEQ ID NO: 23 - DNA, 20 bp, Homo sapiens
  source 1..20: mol_type = genomic DNA
attaccttgttgcaaggtac

SEQ ID NO: 24 - RNA, 20 nt, Homo sapiens
  source 1..20: mol_type = genomic RNA
gtaccttgcaacaaggtaat

SEQ ID NO: 25 - DNA, 20 bp, Homo sapiens
  source 1..20: mol_type = genomic DNA
taccttgcaacaaggtaatt

SEQ ID NO: 26 - DNA, 20 bp, Homo sapiens
  source 1..20: mol_type = genomic DNA
aattaccttgttgcaaggta

SEQ ID NO: 27 - RNA, 20 nt, Homo sapiens
  source 1..20: mol_type = genomic RNA
taccttgcaacaaggtaatt

SEQ ID NO: 28 - DNA, 20 bp, Homo sapiens
  source 1..20: mol_type = genomic DNA
accttgcaacaaggtaattg

SEQ ID NO: 29 - DNA, 20 bp, Homo sapiens
  source 1..20: mol_type = genomic DNA
caattaccttgttgcaaggt

SEQ ID NO: 30 - RNA, 20 nt, Homo sapiens
  source 1..20: mol_type = genomic RNA
accttgcaacaaggtaattg

SEQ ID NO: 31 - DNA, 20 bp, Homo sapiens
  source 1..20: mol_type = genomic DNA
ccttgcaacaaggtaattgg

SEQ ID NO: 32 - DNA, 20 bp, Homo sapiens
  source 1..20: mol_type = genomic DNA
ccaattaccttgttgcaagg

SEQ ID NO: 33 - RNA, 20 nt, Homo sapiens
  source 1..20: mol_type = genomic RNA
ccttgcaacaaggtaattgg

SEQ ID NO: 34 - DNA, 20 bp, Homo sapiens
  source 1..20: mol_type = genomic DNA
aatcgattataagcaaaagg

SEQ ID NO: 35 - DNA, 20 bp, Homo sapiens
  source 1..20: mol_type = genomic DNA
ccttttgcttataatcgatt

SEQ ID NO: 36 - RNA, 20 nt, Homo sapiens
  source 1..20: mol_type = genomic RNA
aatcgattataagcaaaagg

SEQ ID NO: 37 - DNA, 20 bp, Homo sapiens
  source 1..20: mol_type = genomic DNA
aaaattgaatattccttttg

SEQ ID NO: 38 - DNA, 20 bp, Homo sapiens
  source 1..20: mol_type = genomic DNA
caaaaggaatattcaatttt

SEQ ID NO: 39 - RNA, 20 nt, Homo sapiens
  source 1..20: mol_type = genomic RNA
aaaattgaatattccttttg

SEQ ID NO: 40 - DNA, 20 bp, Homo sapiens
  source 1..20: mol_type = genomic DNA
aatattcaattttcctgcaa

SEQ ID NO: 41 - DNA, 20 bp, Homo sapiens
  source 1..20: mol_type = genomic DNA
ttgcaggaaaattgaatatt

SEQ ID NO: 42 - RNA, 20 nt, Homo sapiens
  source 1..20: mol_type = genomic RNA
aatattcaattttcctgcaa

SEQ ID NO: 43 - DNA, 20 bp, Homo sapiens
  source 1..20: mol_type = genomic DNA
aatcgattccagctctttta

SEQ ID NO: 44 - DNA, 20 bp, Homo sapiens
  source 1..20: mol_type = genomic DNA
taaaagagctggaatcgatt

SEQ ID NO: 45 - RNA, 20 nt, Homo sapiens
  source 1..20: mol_type = genomic RNA
aatcgattccagctctttta

SEQ ID NO: 46 - DNA, 20 bp, Homo sapiens
  source 1..20: mol_type = genomic DNA
gattccagctcttttatggt

SEQ ID NO: 47 - DNA, 20 bp, Homo sapiens
  source 1..20: mol_type = genomic DNA
accataaaagagctggaatc

SEQ ID NO: 48 - RNA, 20 nt, Homo sapiens
  source 1..20: mol_type = genomic RNA
gattccagctcttttatggt

SEQ ID NO: 49 - DNA, 20 bp, Homo sapiens
  source 1..20: mol_type = genomic DNA
cgattccagctcttttatgg

SEQ ID NO: 50 - DNA, 20 bp, Homo sapiens
  source 1..20: mol_type = genomic DNA
ccataaaagagctggaatcg

SEQ ID NO: 51 - RNA, 20 nt, Homo sapiens
  source 1..20: mol_type = genomic RNA
cgattccagctcttttatgg

SEQ ID NO: 52 - DNA, 20 bp, Homo sapiens
  source 1..20: mol_type = genomic DNA
tggaatcgattataagcaaa

SEQ ID NO: 53 - DNA, 20 bp, Homo sapiens
  source 1..20: mol_type = genomic DNA
tttgcttataatcgattcca

SEQ ID NO: 54 - RNA, 20 nt, Homo sapiens
  source 1..20: mol_type = genomic RNA
tggaatcgattataagcaaa

SEQ ID NO: 55 - DNA, 20 bp, Homo sapiens
  source 1..20: mol_type = genomic DNA
tttcctgcaagaatatctac

SEQ ID NO: 56 - DNA, 20 bp, Homo sapiens
  source 1..20: mol_type = genomic DNA
gtagatattcttgcaggaaa

SEQ ID NO: 57 - RNA, 20 nt, Homo sapiens
  source 1..20: mol_type = genomic RNA
tttcctgcaagaatatctac

SEQ ID NO: 58 - DNA, 20 bp, Homo sapiens
  source 1..20: mol_type = genomic DNA
ttacctgtagatattcttgc

SEQ ID NO: 59 - DNA, 20 bp, Homo sapiens
  source 1..20: mol_type = genomic DNA
gcaagaatatctacaggtaa

SEQ ID NO: 60 - RNA, 20 nt, Homo sapiens
  source 1..20: mol_type = genomic RNA
ttacctgtagatattcttgc

SEQ ID NO: 61 - DNA, 32767 bp, Homo sapiens
  source 1..32767: mol_type = genomic DNA
gcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgaggtgggttggcgactaaggcgcaccggtgggcactgcggggacagcagggccccgcgcgcagggaagccgcccggatcgcccggaaccgggcatcttccgtggcgggtcagccgagagcccgccgggtggtgctgagtagggagtcccgggctcggggctccgcgggccgctttcaggagcagctggccttggcaccgagcgtgcccgcgggaggcgggggggggcgctgctcggtggctctgctgcgtagccggtgaacacttggcaccgatgcccgccttctgggcaaggtgccctgagcccagcccctcgccgggctgcagcccaccctcggcgcgctcagcccgcttcaccgcttcagggacggaatagaactcgcagatgcagggtgtcgctgacattttcaactttttctgcggtttccgcccgctgtctctgacccgaaagtgcccccggacggttacagaggacacttaagtggtttgcaaagcctgtggtaggggaggagggtgtagaagggccaaaccacggaacttagttttattcatttatataaagcagcactccgattctttttgcgcggcctgaaatgcatgtgaccagagaagtaattaacaaaacaatgtcaacttctaaaaccgagacattacttagatgataaggcgcagcaactcggtgaatctgtacaaaccttggaaaaaaaacacattagtctatgggaccttccagttttctcatgctcctttccagctactaacctctcctaaagggaacaaccactttttggatttgattcccaggcctcgctttcaccgggaaattatcgttgcttgtaaaacagaagaagccgggaaggcaggcagggggagctgctactttacactctgtgctttgggatagcaaaatcccgcatttaagcaatccgaggaaacgagcaaatagacctccctcgcctctccgagcacactcaacagttccggttgcaaaatgtttgcctcctgggcttcccagcgtcccgttagttgttctatttacacataattagatacttaatggagagagaaactagaagttgaggcgttcctccaggctgtattgtaaagtatgaagtgaaatccaaaatgaaatggtaatgttagaaagcaacctcattaaaaaaaaaaaaagtaacactggtcttgaagatctttcaatgtgagtacataaagatctatctcatttcttttgacagcccatagtatttcataaactagatgtaaccatttcctattgacaggaaattagcttgtttccaatttttcaatcccattcattcatccaacaagtatctgttgagcacccactatgttccagacagtgatctagctactgatgacacaagagtgaatgacgaagttctcactcatgaaattttcaccttagttgggagaaacatgatgcaatgaaaatcttcatacatacattgtgtgtacatatgggagtatttctgtaggatagatttctagtgatgaaactgctgagtaaaagggagaattatgcatattttaagttttgatttttccaaattccaggtattccatatatactccaaaatagttgtgccattttactctcccatcatcagtctatcagaggggatgctttcccacaatctcttgaatgctgaatattttcaacttttttacttaagagaaaaaagagcatctaattgttccctcagtatcagtgagtctaagcatcttgtatatgtttatttgccatttatatttttttctgtgattttcctgtccagatacttgatactttctattgagctgcttatttatttcttatgggagatttttatatattttagataatattctctctctcacacacacacacacacacacacacacacacacacacacacacacacacatacagtcttacagccacatccctgaaatcttgaccttgtgaacatgttttactggcagcactctggactcgatcattgccttgagactatttcttttttgatattctttggaaagactaacaatgacagttttattttcaaacccaacaaatcctggcatggaaatgtttgctcttgattctgcttttaaaaaaataaagaattattttctctctttctttctgcaccttatcagaaacagctaaaagaagtgagttgggccaggcactgtggctcacacctgtaatcccagcactttgggaggcccaggcaggtggatcacttaaggtcaggagtacaagacctgcctggccaacatgctgaaactccgtctctactaaaaatacaaaattagccgggtgttgtggcgcgtgcctgtaatcccagctactctggagactgaggtgggagaatcgcttgaacccaggaggaggaggtagcactgaaccaagatccagcctggccaagagagtaagactccgtctcaaaaccaaaccaaaccaaaccaaaaaaagaagtgagttggcactttcaacattctgcctggaaatctccttaccaaacctataagatcattaggtatattttctgcactttgtattgtgacaggtgacagtgttaccaaactttttaccaggacataatagggtctgcctttcttctagttgctaacaatttcccccaagtccatcttgcctgcactaacagtctcctttagacctctcctctcctgcctgtcacacattcctagtactaatgctacagtatagtagtaagggtctccagagaaacaacatttatataacataatataaatacattaatagagagaaagagattttaagaaattggcttatatcattgtgaagtcgggcaagccccaaatctgctggacaggccagcagcctggagacccagggaagagttgatgttgcagctggagtccaaaggcagtctctggcagaattctcttttacttctgggaccttggtctttctcttaaggccttcaactgattggatgaggcccaccacattatgaagggtaacatgctttacaccgagtctcctgacttaaaatctaaaaaataccttcacatcacaactagatgtgtttgaccaaatatctggataccatgcctgggcgaattgccacttaaaattaatcatcacgtacatgttttaaggttttgttacacaagacctcacttccaagtatcactttctgttttggtcatcagttgctgcataataaacaaccaccctaaaatttagtgacttaaaacaacaatcatttattgtctgccatggttctgtggtttgactgggatcagctgagtggcctgtttcacttggtgtcagctgggggtgtaggcatctgcaacattgtcttggcaggaacatccaagatgtcccacttaacacgatggctcctgggctcagctgggctggtcaggcctcccttcctctctgtgttgccacacggcctctctctatccatgtggcctctccatatggtctctccctggtggaggtgaatttcttcaaggtttctaaactctcaaaagtggagcctggcaagcccactcaaagcttcagatccacaactggcacagcttcccttccacagattctataggttaaaacaatcaccggaccagcccagattcaaaagaaggagaaacaaactccacccctccatggaggaagtagcaaaaataatgtagacagtttttaccctttacatctggatttgttagcttttctgttttcattttcttctggtctttcttttgtccttctcttacacttattttattaccttttatcaattagcttttaagatgataaaaatctaatacatgcctcctggaatgtcttcttgagcctagggacttttgtttaatgatatatcttgaggacctagaatagtgcctgaaatacaatagtcattaaatatttagctgaattaaatgaatggtatataagccagggtattaaaaataacataaacaaagttgtaataaatatacttccccagtgaacgacctaataccattactccccaaaccctcaatttctgtcttgagcatagaaactgttaatttttcctttgtgtagtaggtccttagtatttctttagaggttgtagcactttatcttcctcactgttccttttccttggttgtccttttccaaacatctctcaacaatttctcccaagtccatcttgcctgcactaacagtctcctttagaactcccctctcctgcctgtcacacagtcttagtaatagtgccacagtatagtagtaagggtctccagagaaactcactttctgcaagttttttagtgtgggtaggtgagtattgaagcttgttcttggcgttaccaggttggttctttgagttgaaccaggggcattacatgcggaatattcctgaacagatcacctctggttctgctgtctcaagggccacacacaagagctgccttctgaccaagatgtctctgggcacatgagacctgaaatacacatggccaagactcaacaaagcgtttgctgactgtcagagctgacagcatctcggtactgtgggagggagcccagtgtctggtgatagtcaggacggacccaggtgatgtcaggggtggggtggggcctgcaggagggaatggagagccagcacctaggggaagctgggaatttaggaagatatccagagagtgtaacttcagttccactaatctcacctgggtgaaaaccagccctctccatggatgatggtgattgcaggtactgacgatagctgcagactgctggtggtctaggcaagcatgcagggatgggagcaggcatttctgagagctccccttatccctgccccaagacaaggtgggggccctgtggggaaggactattttattcacttctgcatctccagttgtctaacacattgcttatcacctaacagtctcttaatagaagcttgctatattgagctgccttgagtccacatcatgctggttgaacataagtagaaattgtgggagacttaatcagagaaaatctttcatctgtcttaccttacgttctaatgatcacttctgtgcccacaaggaccatcttttctgattcatgttcatgtcgatttcttttttatttaacttcctcttcaaaatttctggcaggatttcttgggagccaattctccatttttacttccccatgcctcccattttaatgactgtaggattttctcagggtctactcagcaaaacttgttagtacaacatgagcaaaacagcaaatttatgcaaacacttagcaaagacttagctgtctgcctaatgctagggtgagacatgggagattcacaaatgaagaaacacaagcattgatctccaggatcctgcagtgggaggtatgcggaggaccaacctgggaacagagcaacgcaacaccatgtgatctgtaatgaaaaagaggcctgtacacagccaagaggtcactgaggaaggagccattgcttctgtgcagttagtgctgcgctagattctgcagggatgtaagaaatgtacccctatcaaacaagaaagactatgtgaattgctgaatatgtgagtggagtaccagcacaatgccatggagatgcagacgtgccccatgtggtggggcagagtcaggaagcactttaagaaagaaatagcatttcaggtcttcctttaaaggtagaatttcaacaagagggtgctctggagagtgtgtgttaccctgctgagaaaatcctggcggtcaggtaagatgctactgccagggaagattggccaattgattgactaaacccttaaaggtttggggatcttggggaggattctgctggtgagagggtctggacttcctcttggtctgtccacagctggaccttctcagcacacaagagactatgagggtgaccattttgcacaggacagaattccagcatgtttttcccctggagtgatggaatgaccacctgctcaacatcagtgtcctcactgagaccatgagattcagtagagtgctggaaagcttcatgcttacctgtgtcttcttaatgcttagtgttatgattgaaggcttccttcagtcctaccttttgttctggggttctaagaatcttaggtgcaggcgaggcacggtggctcacacctgtaatcccagcattttgggaggctgacgtgggtggatgacgaggtcaggggattgagaccatcctggcaaacatggtgaaaccccgtctctactaaaaatacaaaaattagccgggtgtggttgcgtgcacctgtagtcccagctactcaggaggctgaggcaggagaatcgcttgaacctgggaggcggaggttgcaatgagctgagctcacgccactacactccagcctgggtgacagagcaagactctgtcttaaaaaaaaaaaatctcaggtgcacctgagacagattgaatgtggaaggggaagtgaaacaggccttccaggtgtggggcctgggtgctgctatagttacaaatggggaagtgagactataggtctcagttacctgtggaaggaagggtagagtggagtacttacgcaaattagctaattctgggagcttggggtgctaccagggtatcagggagaatacagccagggaatagaatcttcttgaagcaaaggctgtttggaagcccccagagtggatgaaaaggctcagtgggaaacaacagatatcaggagagggagaagaagatacctatttctataccttttggccttgtgttttgcttcagacactgttcccagcaaggtcagtggaacccactgctcaaaacacacacttgctccttgttctggtgtcataatagctctgcaagcagtggtggtgttcagcctggagaacgttccttttctttttttttttatcacaataaacactcatggcttctctgcttcttcctttcttctttgtcttaggactcttgaaaaacagctgccaaatgtcagtttagatattttggagggaaaaaagttgggaatcaatgtttacaggttgcctgcaatgtgctggaaactacatagttggttctttttaaactttctctgaatcctgtcaggaaagttccagcaatcacatcttagtgggtccggaattcgtgggttcttggtctcactaacttcaagaatgaagctgtggaccctcgtggtgagtgttacagttcttaaaggtggtgtgtccagagtttgttccttctggtgttggacatgttcggagtttcttccttctggtgggctcgtggtctcgctggcttcaggagtgaagctgcagaccttcacggtgagtgttacagctcttaaggcagcgcgtctggagttgttcgttcctcccatctggagttgttcgttcctcctggtgggttcatggtctcactgtgctcaggagttaagctgcagactttcgtggtgagtgttacagctcataaaagcactgtggacccaaagagtgagcagcagcaagatttattgcaaagagcaaaagaacaaagcttccccagtgtagaagtgtagaacgggacgccaatgggttgccagtgttggctccccccagcctgcttttattcccttatctggccccacccacatcctgctgattggttcattttacagagggctgattggtctgttttacagagagctgattggtccgttttgacagggtgctgattggtgcatttacaaaccttgagctagacacaaagtgctgattggtgtgtttacaaaccttgagctagacatagagtgctgattggtgtatttacaatcccttagctagacataaagattctctaagtccctagtagattagctagacacagagcactgattggtgcatttacaaaccttgagctagacacagggtgctgattggtccgtttacaaaccttgagccagacacagagtgctgattggtgtatttacaatcccttagctagacataaatgttctccaagtccccactagactcagaagcccagctggcttcacctagccgattgtgcaccaagtcagcaggcggagctgcctgccagtcacctgctatgcacccgcactcctcagcccttggacggtggatgggacgccagggagcagggcgcggtgctcgtcggggaggctccagcggcacaggagcccacggcagggagggtggggggaggctcaggcatggtgggctacaggtcccaagccctgccccgcggggaggcagctgaggcccagcaagaattggagcgcagcgccagtgggccagcactgctgggggacttggcacaccctccacagctgctggcctgggtgctaagcccttcactgtccggggcctgctgcgctcgccggccgctcagagtgcggcctggggagcccacgcccacctggaactcgcgctggcccacgagcgcctctctctctacacctccgctcaagcagagggagccgactccggcctgggccagcccagagagggtctcccacagtgcagctgtgggctgaagggctcctcaagcacggccagagtgggcgcgcagaggccggggggcactgagagcgagcgagggccaccagcacgttgtctcctctcattagggttggggaaatggaccctgagagagattaagtaatttggtgatattctatagtcactctggctatgtaatttatggagcctagtacagaatgaaaatgtggggctcatttttcaaaaagcaggaaacagcttttcctttcttccagggtctcttcctccacctgccatgctggtgtttggttgctatttaatgttgagccctcttgggcacagggatacttgcagggacagggtgtctgctcatttttctgtagacctcaaaggtgagtcctgaggcttcagggtcactggcctccttttagggagtcacgacgccttgtctttgtacttcaggaatgattacgaatctttgtaggtaaagcggcagaatgccacgtcctctcctggttgccaggacgtgttccttgtggtttaattgccgggtctgccctgcagaccctggctgagcgacagatgaaaggagtactcagacacaggtacgcagtgaaagagcggctaggggactgccgaagagtcagcagtctcaataaactggagctgctcacttttattcagtacagacataatgccgaaagcctggagccaacgcagtctgtgggtaattaacattgttgttccgccgtgcagggagcagtctcgccagaggatgatgaaaggttggtttccggaagtaaacaagcttatttagacaaactcccctacattcccttgtacccactcctcgccctctgcgtcagggtaagagaacagctgccttcagcttattctcccccgaagctttgcagagccttctgacctttcaaaaggtcttcttctttccctatcggttctcccactactctgactgatctcctatatttgatctcaccttaacaatcacttcttagagctgggtcaggaagtatgcagcatgcacctggcactcctagtactgtgcccatgatgggcattgctgattgttcagagcatattggatgagcctggttcagcctcagaatcttccacccagtgcaccatggagatgctaccaattggttggagttgctctgagaggtgacatttccttgtgattctgcattagaaacatgttgtttgtcagccgaaacagggaaacctgacacgttatccgcccccaggaagatcccatcatcattccatgcaccttcagtcctgggagcttactttaaaaaaaagtgactgacatatgagcgcaggtccccaaacagaggggaggcaggatgagaagccagatgaagagagtcaaggtcctggggctgcttagcttggatgaatctgatgggaggtggggtgcatctgagtgttctctgctgatgaagaacagacttgttgcacgggggtaggtgtgtgctgtgtaaacacacatcagaatcaggaccccgaatagtgaataggcaagagtaacagctgaatttgcccagctcatcacaatttaacatcagttttcaaaaaggtaagagcgtggctttcatagcatgcagaatcaacacacatcaaagattgatttactcatttatgaaggaatcagcaaaatgacaaacttagttcagagaatattttgaggctctgagtagatataaaactggttaatgtttctcagggcaataaaaagctataaacgttggggatttcttttttatcagacagaaattatttgcatacttaacagaaaagatctccaagttaccatctaacttcataaggttcgaataaaacttcatagagttattaatgaatggtaaatagaaaagacaaatatatgttttaccagataattaagtaattcttggtaaacctggcaaacagtaccccagtgtgactctgaaaagacatgctgcccatctttttgccttatttccacgttttaggtatttttgtaagatatctattcaataaatatgtattgagctcctatgatgtcccagaaactcttttagatcctggagatatagcagtaaacaaaacagatgaaattcttgctcacatggaacttatattctagtaggggagacagacattgaaacagaaaaatacatagtatggcagatggtggaaagtattaaaaagagtgctgtgtagtgtttacaacttactcatttatgaaggaatcagcaagatgataaactcagttcaaagaacatcttgaggcaccgagtacatttaaaagtggttagtttctcagggcgataaaaagccataaactttggggatttcttttttaggtatggaaacctaaagtaaagaagatgctatggtttgcacatttgtcccctccaaaactcatgtttgaaatgtaatcccagaagtggcaggatgagagattggccctttaggaggtgactgggtcatgagagatctgccctcatgaatggattaatccattcatggattactgattaatacgctaatgggttaatggatcaatgggttatccttggaatgaaatggcaggctttacaaggagaggaaaagggacttgagctagcatgctcaccctcctcaccgtgtgatgccctgtcctgcctcaggactctgcggagttctggtaagcaagaaggctgtcaccagatgtgtcccctaaaccttggactttttggcctccataactttaaggaataaattcatttttaaaataaattacccagcttcaggtattctgctataagcaacagaaaatgactaaaacaggaggctttactggaaggtgtcctcttagcaaagacctaaagaaagagggagagtgaaacatagaaatatctggggagaacatcctaggtaaaaggaacagcatgtgcaaaggccttgaaaagcagcaagccgctctccctctccctctccctctccctctccctctccctctccctctccctctccccctctccctccccctccccctccctctccctctctctccacggtctccttccacggtctccctctgatgccgagccaaagctggacggtactgctgccatctcggctcactgcaacctccctgcctgattctcctgcctcagcctgccgagtgcctgcgcacgccgccacgcctgactggttttcgttttttttttttggtggagacggggttttgctgtgttggccgggctggtctccagctcctgaccgcgagtgatccgccggcctcggcctcccgaggtgccgggattgcggacggagtctcgttcactcggtgctcggtggtgcccaggctggagtgcagtggcgtgatctcggctcgctacaacctccacctcccagccgcctgccttggccccccaaagtgccgagattgcagcctctgcccggccgccaccccgtctgggaagtgaggagcgtctctgcctggccccccatcgtctgggatatgaggagcctctctgcctggctgcccagtctggagggtgaggagcgtctctgcccggccgccatcccatctaggaggcgaggagcgcctcttccccgccgccatcccatctaggaagtgaggagcgtctctgcccggccgcccatcgtctgagatgtggggagcacctctgccccgccgccctgtctgggatgtgaggagcgcctctgctgggccgcaaccctgtctgggaggtgaggagtgtctctgcccggccgccccgtctgagaggtgaggagaccctctgcctggcaaccgccccgtctgagaagtgaggagcccctccgtccggcggccaccccgtctgggaagtgaggagcgtctccgcccggcagccaccccgtccgggagggaggtgggggggggtcagccccccgcccggccagctgccccgtccgggaggtgaggggctcctctgcccggccgcccctactgggaagtgaggagcccctctgcccggccagccgccccgtccgggagggaggtgggggggtcagccccccgcccggccagccgcccagtccgggaggtgaggggcgcctctgcccggccgcccgtactgggaagtgaggagcccctctgcccggccagccaccccgtccgggaggggggagggggggtcagccccctgcccggccagccgccccgtccgggagggaggtggtgggggtcagccccccgcccggccggccgccccgtccgggaggtgaggggcgcctctgcccgtccgcccgtactgggaagtgaggacccctctgcccggccagccgccccgtccgggagggaggtgggggggggtcagccccccgcccggccagccgcccagtccgggagggaggtggggggatcagccccccgcccggccagccgccccgtccgggagggaggtgggggggtcagccccccgcccggccagccgccccgtccgggagggaggtgggggggtcagccccctgcccggccagccgccccgtccgggagggaggtggggggatcagccccccgcctggccagccgccccgtccgggaggtgaggggcgcctctgcccggccgcccctactgggaagtgaggatccctctgcccggccagccgccccgtccgggagggaggtgggagggtcagccccccgcccggccagccgccctatccaggaggtgaggggcgcctctgcccggccgcccctactgggaagtgaggagcccctctgcccggccaggaccccgtctgggaggtgtgcccagcggctcattggggatgggccatgatgacaatggcggttttgtggaatagaaaggcgggaagggtggggaaaaaattgagaaatcggatggttgccgggtctgtgtggatagaagtagacatgggagacttttcattttgttctgtactaagaaaaattcttctgccttgggatcctgttgatctgtgaccttatccccaaccctgtgctctctgaaacatgtgctgtgtccactcagggttaaatggattaagggcggtgcaagatgtgctttgttaaacagatgcttgaaggcagcatgctcgttaagagtcatcaccactccctaatctcaagtacccagggacacaaacactgcggaaggccgcagggtcctctgcctaggaaaaccagagacctttgttcacttgtttatctgctgaccttccctccactattgtcctatgaccctgccaaatccccctctgcgagaaacacccaagaatgatcaataaaaataaaataaaataaaaaaaaggaatgaatcaagaaaaaaaaagaaaagaaaagaaaagcagcaagccagccagtgtgtttggaatgttctcttatggaaaatttcaaagatatgtaaaactagggctaataatacaattaacccctacttacccattacccaacttcaacaactatcaacattctgctgttcttatttcatctatttacccattaaaaaaaaagtgtactttaaagcgaattccagagtttgtataattttgtctgtaaatctttcagtctgtatctctaaatctttcagtctgtaactctaaaaagaacattaaaaaacacaactatcataccatcatcctacctgacacaactgagtaatttttcatatcatccaatatcgatcagaagtttaattttccatgattttctttaaaatgtagttttataattgatttgttctaatcaagatttgcacattgcattaaaaatatatgtatcttaaatttcctttaatttacaaccatttccctcccccatttaagagtgcatattaatttattaaacagactgggcaattcatcttgtagaatttcccaccttctgggtttggccaattacatccttgtggtgttatttaaaatcctcctctattccctttattatctgttgactgacagcttggccaatcagaatcacttgaacgagctgattacaccccctctttctgaaacgttttcttcccttgagtctgtacacactgttggcttccaaatttatatctccaacccagacctctctccagtacttctgcttgctactgtgtttttcataactttctgatttttttagtatccaatgctagaaaatatatccattttgagaaagagacaaagtatgagctcatattgataatttcatttcaaagtaaagggaacaaagtttttatttaacatgttaattttatgcttgtttctcttttacactgaaaatcttagttctcaatgacattaatataattatgtatttacttcccatatatatgttgtatataattgttttatatatacatatgtgcatatatattattgctaatgaaacgtctactgaatgatgtaagttttctctgtgattcttttggtccttgggacacaggacttatcccactagtgatgtgtagtcaaaatactatgtaccagtgtttgataccttaattaaaggatctctctggctgctgcattgagaacagatggtagcaggacaaggacatcagcctggaagccatctggaagctctttagtgattcagataagatttgattttggcttggatggtgatggatgatgttgagaagtgggtggattctggatatgttttgaaagtaggttccatgtgatttgccatgggctaatatatggaatgtgagagaaaaagaagaaacaacaatgcttccaagattttggggcagaggtactgaaaaaatgaatttccatttatcagaaagagaaagactgtggtaaagcaagttgggaaggaaacagcagctcagttttccacattcagtaaccctcccttatctagggtttcactttctgcagtttcacttatccatggccagccacagtctgaaaatattaaatggaatattccagaattaaacaactcgtaagttttaaattgagcactcttctgagtagcgtgatgaaatctcatgatgtcctgctgtcttctgccctggatgtgaatcatccctttgtccagcatatccatgctgcatgttgtacctgcctgttacttgcctaagcaagtggatggctacttgcttagtgtccatctcagttatcagattgaaaaagcagtacatatacatagggtttagtactatctgcagtttcaggcatcactgagggggttggaacataaccctggcagataagggagactactgttttagtggagatgttgaccagaagactggttcatatgaatatgggggtcctggagagaggtctgggctggagatataaatttggaaatcaacagcgtatacagacacaaggaaagaaaacatttcagaaagagggggtataatcagcttgttcaagtgattctgattggccaagtaagatgaagattggaaattgaccaatggatcggtgacttggcaaggtcagtttgggaggagtggtagtgatgaaagcttatgtggaacaaattcaagaaaaaaacagaagagagaggaattagagattgttgtgcacagacaactcttgcaagaggttttgctggccaggagaacagaaatacaagtgaagtagtggctggatgttttccaaaacatgaaacttagttttcataggaaaaaaatggtttttctttttctacttattcaattttgtgcacaatttcattacattatataagtaaaaaccacaagcacgaacatgtttataaataggtaaataaataacaaagtagatagaaacaaaaattctcaggtgtgcaaaagagtagtttattagctgtgtagaagacagaaaacttgcttttatagagggaatgaatagtgttgattagtatagtgagtcagttaaataggtatcagttgaattttttgagacaataagtacatttagaattggctaggcattattcttccattaaaggaaactcttagtaatagattaggtcagaaaaccacaaagacagttttcttgaataagggggtggtgggaaaaaatggatgtaagatgctgagaaaaaacaggaaactcaacaatagatgaaatcaagaattagtttcgtgtgtgtgtgtgtgtgtatttcacagaaaagggctgaaaggggaggaatggttatattcacagatttttgtagttgatcttaatagggaataggagattacctttttttcagcaaaatattaccagcaaatgtctactctggaaatagaggataagaatcttctatttaaagaccaaaaaggtgaaaactgaggtcagaaatttaaagagtaaatgtagcaagatgctaacattcttgttccctctaataactcttcttttttaaaaattataaaagtaaaacatgcccattgaagaatatttggatgaatacaaaaatatagaaaagaatgtaaaatcgcttaagcccccaagtagaattctaaatagcggagaaacccacgatttaaaaaaataaataaggagatccacaaaaggaagatatatacctgcttaagtggacggcctcagcataagtagtcttacagttacaattaattttttttctattatacgatgttttaaaaaattgctaagctcagttgtctactccctgctagaatgtatcaaacatgctgcatccacatagtagacagttattctacatttttggtctttggatgttttgaagcaattagcaaagtttgatttgaagagacattataaatttccgacggcatatttttctctggccatgatccatatttgccttgatattgtccaaatgttcattttatcttatgactatcaagcaaatattataaagttatttgtgaatttggcacttcatagatgaaatattgtgaaattctgaataaaactgcttcattgattatcttggttcctaaaagagatagtttatgagttaaattatctctaacgttgtctaagttggcagtaattaaatctccatgggaattcttaataaaggcaaagaaggactgaaaacaccttgctgaaactgagagagatttaaaaccacaaaaaaaatgtaagatttagttagttgttcacgttttggggtcctgcttttatgtgttaatacttccagataaaattttttcaaaggtagttttgaaccccaggatctgaatatattaatttgtttatctctgaaaactgtatactttgtgtttcccaggtctggctttcttcatttttcctacttgagggaactccttttcttccttcaatatcctcatatcctccttcaatgtttctcagtcaatatctcctctcaatttttccatttaaaaattaaaaattaaattattaacattttaaatacactgaaatgttcatggaatagtagtacaaatacccatttacccaccacatggagttaatagacaataaaaatttgccatatttgttttacatcttttttctagtttttaaagaaataacatgttacaaataaaatcgaaatactctttccgccgattttcttttctcctgtctcagaggcaaatactgctacttgcttctcttgtatctttttagaaatattctctgcatatataagcatatatccatatatttttccacacatagtatctcttccgtgtcttagtttttttcattctacaatgtatcttgatgatcagtgtagctttgtttcattctaataattatatagattccactttacaaatttactatactttggacttgtatctatgaacgtttcgattgttttaaacctttggcaacattacagacaatgctgcaatgaaatccttctgtatgtatacatacgtggatgtgtgtataagatataattctggaagtagaattgctaagttaaagcatatgctacttaattttgaaaaaattgtcgaacttctcttcacaaggcaattatcttcttgaggtaatgagaactcctatttccccatacccatactacccagagtatcttcaaattttctgatttttgctggtctgataagggaacgttacctcagtttaatctttatttgcatttactatgtgtttttgccctgtagagtgaattttacttttctacctaacaatatatttttgtgattttaatgacattacttttgagatttacccatgctaatatataaactctggttctgcaattttaagtgctatatagtatcctattgaatgaccatactactcatttatttattattatctacttgttaattagtctgttatcttttttgtcagtttttgtgggcataaacaatgctaaagcatacatgtctacttgcgcatatgattttttcccagtgtgtttaatagaatgactaaggtagaatgactaaagttctgaggtgtggcaggtatgccatcttcacctttactgggtatgctagtgtgctgcagctgccgtgataaagtactacaagccgggtggcttcaacaatagcattgtactgtctcacagttctggaggctggaagtctaaaatcaaggtgtcagaagggtcggtgccttctgaaggtgtgagagagaatctgcttcgtgccctcttccaagcttctagtaacctcaggtgttccttgtcttgttgatggtgttgtcccagtgtcttcatattgtcttctctctgtttgtgtttgtgtttgtctctatgcccaaatttccccttttcataaggacccagtcgtatgggattagggcttaccctaatgatctcatcttaacttgatcatctgtaaaaccttatttccaaataaggtcattttctgaggtactgggtgttaggatttcaacatcgtttgggggggtaaaattcaatcaataacaacaggtataccagttaagatgtttttggctgcaactaacagaacattcaactgaaaaggtttaaaatatattgtaaattttaaacaaatgttttatagagatgagttctgactgtgttgcccaggctggtctcaaaatcctggcctcaagcaatccttccaccttggcctcccaaagtgctgggattacaggcatgagccacagagcctggccactttaaaatgttgttgacacacataacaagacatccagaagtggggtggtcataggatcggttcagcagcacagtgatggccgaggctctttccttccactcttccatctcccttgagtggctttcagcttcttgtcacaagattgctgcattatgtcctcatatacctggaacagagggcagggagaggggcaagagcacacccccatttcaagtctcttttcatcccaaagtaacatatttcccagcagccctctttcaaccctctctaggcttctccttaaatttcactggccagaattgggccacacggttacccaaaggtataaaagaggcttgagaagtgagaatttgatattttgaatcataataggagacatgctttgccaataggaaagaagggtaaaagaaatcttctggagagtacggaaccaacggcgtctgccacgttgcatatggccaaatggtcaccaaatttgttgtatcactttattttcacaaaattggtgtaaagtcctcattgctattcatcctcgtcatcattcggaattctcaaatttaaaaaatgcccatttccttgttgttttatttttttaattccctgatcattaacataagtaattatctttttatgtgtttatttgtcattcaggtttcttctgtgaaaagcctatttctgtcttttgcttgttcttatgttgaattgtctctacacattcaggattctaatcttctgtcagttatgtggattgcaaataaagtctcccagtctatggcttaactcttgacactatatttatcacgtcccttatttactaaagatttaaatatgatcaaatttatgttttcttttagggttgtgccttttgtatcttatttaagaactgatttctttaatgtgagatcacaaaagggttataaatattctaaaaatgttaaacttttgcttttcatagttagatctttgatccacttggaatttatctttgtgtgtggtgaaattaggaagcccatttcatttttccccagatggatagtcagttgtgtaagtgctgtttatcaaataatccatcctttatcgagggttttctgatgccacttctgttgcatcttgtgtttctttatgtatgtcagtttatttctggactctccaatctgttttatggtctgtttgtcgctataccactgccacgttgtttaaattactaatgctttgatatccaatagaacatgtctgctttctgttacgggagataaatttacatttttaagaagtttatataaatggaatacatatcatttactcttttgtgtctgagttgtatgtctttgtcatgatgtttttgtgattcatccatgttgtatgtattaatagcttgttcctttttattattgggtctattgtgtgtatatatcacaatttgtttatttatctattgatagacatttggcttgtagtcactttttggatacaatgaatagaggttctatgaacattagtgtacaagttattgtatggacatactgacagagcaggagcacagtcctcttggacaaacactgccactttaagttccagctccatttttagcctcatgcatctcagggaaatcacttctcttctaactacaagtagccagaaagagcaaacagtaaaccacagataaaacagctcaggcacagagggaggagggagaaaagtctcttgggtaactgccacacttcaccctcatacagtgggccccagtaaaacagtgggcgttaataaacacattattttcccttcaggtgcactaaaatagggaagctaaaagcagactcggggggtatgcctgcagctgcagaaaaatgtataaaaacagacacacaactctccctccaaaataagcacaacaaaaaacacaaaagcagtccaagcctctaataaactctcctatcctaaatccttaaaaactcttagtctgtaagagagtgtgctgttgacctagctcagccaaaagctcctcacaggttcgttttctctaaaataaacctgtcttaactggcaagccacctttcgtgttttttttcctctttctttaattcttacacatactgttttatttctcttgagtgaacacccagaaatagaatagcagagccatatggtacataagttgattagctttttgagaaaccaccaaactgttttataaggcaattgtatagttttacatgtgtagcatcagtgtgtgaatattctagttgttctacgtccttgttaacatttggtattgtcagacttttaaattttagccatctaaaaatttatagtgttattttatggtggttatagtttgcatttcccccatgactaatgatgctgaggatcatctcataggctttttgaagtatgtgttcaaatcttttgcccatctttaaaaattagggtttttgttttgtacaaatacttggaaattaagcaacatactccagaatgaccaatgggtcaatgaagaaattaagaaaaataaaaaaacttactgaaaatgatgaaaacatgtctaacaaataaaaattgatacacaacataccaaaatctatggaatacagtaaaagcagtactaggaggaaagttcatagtaatgattgcctacgtcaaaaaagtagaaagatttaaaacaacttaacagtgaacctcaggaaactataaaagcaaaacaacaacaacaaaacccccaaactccaaattagtagaaggaagtaaataataaagatcagaacagaaataaatgaaataggttggaaaagtaatacaaaagatcaacaaaatgaaaagttgttttttaaaaaaaattgactaagcattacctagactaactaagaaaaaagagggaagaaccaaataaatgaaaaaggagatgttacaattgataccacaaaaatataaaggatcgtaagagactattatgaacaccaataaattggaaagcccagaggagatggataaatttctgggcacctacaacctaccaagattgaaccaggatgagatacaaaatccgaatagaccaataacaattattgaggaacctcaataataatttttattaaacaacaataaaaagtttcccaattaaaaaaaaaagctcaggactggatggctttactgctggattctaccaaactttgaaaaataactaccaattcttctcaaactattccaaaaaattgaagggaagagaattcttccaaactcattctataaggccagaattaacctgatacaaaaccagacaaggatacaacaacaaaaaaagaattttgcaggccagtatccctgatgaacataaatggaaagttcctcaacaaaatactagcaaactgaatccaacagcacattaataagtttatttactaaaaccaggtgggattcattccagggatgcaagagtggttcaacatatgcaaatcaacaaacataatacatcccatcaacggaatgaaggacaaaaatcatatgatcaccacaatagatgcagaaaaacagttgataaaattcaacatccctccatgataaaaactctcaaacaattaggtttagaagaaggaacacacttcatcttaataaaggccatatatgacaaatccacagctaatattgtaccaaacagggaaatgttggaagttttttctctaaaaactggaacaagataaggatgcttaccctcactactctgattccacatagtactggaagttctagccagagcaattaggcaacagaaagaaataaaagacatccaaatttggaaggaataagtcaaattgaccatgtttgcagatgacatcctcttacctacagaaaaatctaaagactccaccaaaaaactcttagaattgatatacaaattcagtaaagttgtgagatacaaaatcaacatacaaaaatcagtagcatttctatacaccaataataaactatctgtaaaaggaaccccacttacaatagctaccccccaaaaaaaacctccacctaggagtaaatttaaccaaagaggtgaaagatctctagaataaagactacagaacactaataaaagaaattgaagaggacataaaaaattggatagatatcccatgttcatggattggaaaaattaatattgttaagattccatactacccaaaggaatctacagattcagtgcaatctctatcgaattttcaatggcatttttcacagaaatggaaaaaaagattcttaaatttgttaggaaccataaaagaccccaaatagccaaagcaattgtttgttttattttattttattttattttattttattttattttattttattttattttattttatttcacttcatttcattttatttttgagacagtctccctctgtcacacaggttggagtgcagttgcatgatctcagctcactgcaatctctccctcctgggttcaagcaattctcctgcttcagccacctcagtagctagaattacagacatgcactaccatgccctatttttagtagagatagggtttcaccatgttggccaggctggtctcaaactcctgacctcaagtgatccacctgcctcagcctcccaaagtgctggtattacaggcatgagcctctgctcctagccaagccaaagcaattctgagcaaaaagaactggagggatcacactactttactttaaaatataatactatagtaaaacagcattgtattggcattaaagcaacacataaatcaatggaacagaatagagaacccagaaataaattcacatatttatggcctattgattttctgcaacagcactaagaacataaactgaagaaaggacacctttttcaataaatattgctggggaaactggatattcatatgcagaagaatgaaactagagccccatctatcataatataaaaaagtaactcaaaacgaatcaaagacttaagtgtaagaccccaaattatgaaactactaagagaaaacataaggaaaatgttctgagcaaagattttatggataagacctccaaaacacagtcaacaaaggcaaaaatagactaatgggattacatcaaactaaacatttctgtgcagcaaagtaaacaatcaacagagtcatgtgacaaactacagaatgggataaaatatttgcaaacggtttctctgacaaggaattaatatctagaatatacaaggaacttaacagcaaaaagccaaataatctgatttttaaaatcggcaattgatctgaacagacgtttctcaaaagaagacataaaatggccactaagtatttgaaacagtgctctatgtgactaatgattagggaaatgcaaatcaaaatcacagtgagatattatctcacgccagctagaatggctatcatcaaaaagaagaaaaaataacaaagctagcaagaatgcagataaaaggaaacttaaaacatttcagcttttactttagattcaggggttacatgtgcaggtgtattgcatgatgttgaggtttcagaatatgattgaacccatctcccaggtggtgagcatagtacccaatatgtggttttgcaacccttccttcctccttccctccctcctcttatactccccagtgcctagcattcctatttttatgtccatgcgtacccaatgtttagctcccacttataagtgagaaatgtagtatttggttttctgtttctgcgttaacttgtttaggataatggcctccagctgcattcatgttgctgcaaaatacatgatttcattcttctttttgtggctgcatggtattccatggtgtatacataccaccacattttctttatccaatctgccattattgggcatctaggttgattccatgtctttgctactgtgaatagtgctgtaatgaacatataagtgcatgtcttttttttggtagaacaatttattttcctgtgggccatatacccagtagtgggatttctgggttgaatggtaattcagtttttattaatagttctttaagaaatctccaaagtgatattcacagcggttgaactaatttacattcccaccaacagtgtataagcgacaaagaaaattctcacacattgttggtaggaatgtaaattagtacagctattatggaaagcagtgtggaagttccttaacaggctaaaaatagaactaccatatgatccagtaatctcgctactggacctatatccaaagaaaataaaatcatatgtcgaagagatacctgcactcccatctttactgtagtttcatttataataatgaagatatggaatccaccgaagtgtctatcaacagatgaagagataaagaaaatgtgggatatatagacaatggaatgcagccataaaagagaatgaaatcctatcattggtggcaacatggatgagcctggaggacattatgttaagtgatataagccaggcacagaaagacaagtttcatatattctcacttacatgtgggagctaaaaaagttgatctcagagaagtagagagtagaatagtggttactagaagctgagaagggtagggagacagagattgatcaatgaatacaaaattatatatatggatagaggaaataagttttagttttagtgttctatagcattgtagggtgactatagtgaacaataacttattgtatattttcaagtactgagaggagaaaattttgtacattaccagcacaaagaaatgataaacctttgagataatggatatgccaattacacagattcgatcattatgcattgtatgcacgtattgaaatgtcacttcaccccataaatatgtgcaattacatgtcatttaaaagtgataagaaaaattagtttttttgatcttattattgactcgtaggaagcttatatattctgaaaagaagtccattttcagatgtgtactaaaaatattttctttcattttatgacttacctttccattttctttaatggaaacttttaaaaacttttaaaaaacaaagttttaaaaaaatctagttaaggtccagtttgttaacttttttctaatatggcttgtgacgcttattccttctgcctagaatgttcctgggatgtttgtggagctaagtcctcgcttccttcaattctttactcaaatatgtccaccctatttaatgtcaactgtccaccgtatttaatgccaccctatttaatatcactaacacctcctcccccctcactcttgacattcattctagtctattttacatttttttctcatagaactcataaatttctagcatgctttataacttacatattcattatgtttattgtttattgtccgtctttgttccagtaaaatgtaaactcctagaagaacagagacctgtgttttgttcactgatgtaccctaagtgctcacaagtgtttctagcacctagtatttgctcaataaatatttgctaggttgatgaattaatgatttctaagctttccttcagcctgaagagttttctgattgtaagattctacttagataatcctaattgtctcagtgactctcaccagtcactcacttctcccacaaggtggcagtctttaccttcaacacaggttctggtagcctcaaatttgagaattaatagctgagttaacctgcttgttttctttgagcccagacagcctgccctatgggaactgacagctgtaaaatttaaaggacgagtgtaattaccctgcaagatctgagtgcttttaggcaagaggatttagggggtgagagttttcctggagagggacacattatgaaggtgatattgcttaattgatggggactttgaaacatagttgctctttgtgagaatggtataggtttagagagaggtgctagcacagagctgtgacacctgaagtaggctgaccgcagacaaattggatttaaccaccaaatatatctgtgttttcatgtcttcctgccccgtgccctcttatctgactcactttaccccagcactggggaataactgtgccctattctggtcctgacccttttgtaccatctagggaaatgagaactcctcttggggtctcagatcctcatttctgttagaaccaatcctattctgtgggtagggccatggttgtaaatttcctgtgggaggcagcattgctttgcaaaaagaacacagtttggcatgtgaggcagctctgccacttggacaaggtgataacgctttagtctctttatttctaaaacagggaagatgctaataccccgcccatggactagtatgagatttaaatggcaggtacttggcacagtggcaggtggtgaatgctctttggtgatcatgactatccctttctcctggtagtgctgcctcctccctctgagcacctggagtcaatccaccttgggtaggtcagagaaggcagaagaaagtggtgggaggtgaactcgacggaatgatgtacagggcgatagggtgagtgagaggtctgggatctattggcaggagcagaatggtaggaaagggaaaacatgccattgaccttgaatcttgacatttgtgcccatcctatgctgtgttgagcctcaggtcaccgtttgcggaggtgagcagaaaactgctaacagatcgaggcttctccagccttctaggtaaactttcatcagtgggttagttgtcttgttcagaagctgatcacggagctttggccaagcataaacactgattatggcagtccaattgtcataatccctttgattctttaatatcaccttcaagattgtttgttattgtcaatgcccccacaacctaagaccaccaggaacacactgtaattgaaaaaggtgggtttgttgctctctgcaagaagggaggacactcagcgtagagactcatgagggtggggcaggttttatccgatgacgttagaaaggacttactgaggatttgggcttgttttaggagatttgggggaaaggttcaaggagactggcttttcctggatgctgccaggaagtagtgggatggtagtaagtctgtggtaggatgtttaaataaatttcctctactgggctggaagaatgagaaggctgaagctgtaataagtaaagaagtggcagtcactcctatcagctatgataaaaggatgtttggctattactttatggtttggatgctatttttgcttgtgttcacatcatggtctatcatggtgataggccacgtacacagtggccttgtctgatgctggtgtcccatggagttgattatgctcagctgaaggacactaaggcccaactgtgggggccaggccagctcctgagtgtcagggggggctgcactgctttgtcattatcaacatctccacatacaatacagcctgtgcatgtgaggtcccagaaggaaggagctaaagcagctagactgggatcactttactcaatttgaggagaggagtgctcttgagggaaccagaggaaggcagatgagctggtctgatctccttttctattggagctctatgcagatagactgaaaatatttgctaaaacaaagagctccatctctagaatacccttagcaggatgtccttgattaaaggattatttctgaaaactaaatccagaatccgtgaggcatgattccctggaagatcatgtaagctgtacaattctctatggaataaattggagacttcatcccttagatccctttgactgtgtgaggaaccccacgaaacctcacttagtgactttattcattcttctgggcctgaaagcatatgtctcatgcaggaaggaaggcaggaccagtggggctttgcaggttgtgccattctctccttgctttgcttgagactctttattacctgtctccctgacattatttgtagtgtgattctgggtgtgttctgtgattcttgtgactaggtatccaattttgcttaaggatgcaaggaagtgtttggggagaaagctctattggaagaggtctgtagtcctagcctccctccccaccccacatttcacatcattagacctcagcacatgggtctggggcaccaacactgtcttacctgttacacagtgtggtctttatctggatgagggatgcgaaaggatacattgtgaccaagagacctgggagaggcacaaaaataacaggtgaccaccaagggtgcttggacctgagatgtttccatttcctaagaccctccaagattctccaacatttggtatagttgcccaggcaatttaacaagaaaatcaaatttttgttatgacccccattgtaatttatgcttatcgcagaaaaattgagactataagaaggagaatagaaggtcacaaaaccactctatactagtccagggatagctattcttacaacatggattgatcagtgtggggtgatttctcctagtgttttttggacagagaagcattgaagatgccctggttttaaggtcttaggatgaaggaattatagttgaacagttcaaaatgatgttatgaattactttcagatttgtttgcttgattgcattagccttgcctggccctacggtaactatttggttccatcatggtggctgagtaggtggctctggaaaaagagctattcaagaaaagcttttctttctctaaaaatattgtagggggctcgccctctgttcttggaagcaacgtttgggatggcctcttgggaggctgtctggtgaagtgtctaggggtatgtggtctggacttggacaggacaagatgcaaattctggctgggatattctagttgtggagtgttgggcaagttacttggtcttctgagtttataggtaaactggagataataggtatgtgtgaatgaggatccaatgagatgcctgtaaaacacttagccagatggctgcgtgaagaaagcacttggtaaatgctaattgttgtggttgttataattagtacaatgattagtcattgctgattgttggctaactggcggtaagaaatgaaagtaaagtaaggcagtagcagctgagggaggtggtggagggaatcaggagacacttggaggttctggttctgcctgagtttaagtgctggggaaaactagttgaataactgctggtctaacatttaacagctgtgtgacctcgggctagtcacatttcctttataaacttcctttttctcatttgcgaaatgaaggggtttcgttaggttacttctcatcacccctggttgaccattagaatcgtttgggagacctttttagaaattcttggttctggggtcttccattttccccattctcacttggtgggtctgaaagcaggcactgcagcttttccaaagctcttcaggtgaccttgaagtgtggccagggctgagaacctctgacttccaacagcacttctggtttaggaaggagcaaatcaccggcacagaatgagctctcaggaacggctgctgagctagtaattgccgtgacactgtctccctgtcccaactgcaggcacccctagacgtctcctgatgaagacttccaattttggaacagaagaatctttgaaaaaaatattattgaacttccagaaatgattcattccttctctgctccttctttagttggaaagatctgcccccatccctgtctactgcagtcccaatccctttttatttcaacatatatatccaaaccaacaaaaaaattgactcgcacaaccaaggtgaggtgtttggctttaaggataaaataaatagtttcataaaacctgcccccagatttctcattgcctctactcattttctctaatttgtagggcacactgaaagctcggattcataagatgtagaaagggcagagagttaagttacaaactcttcctaggtctgtttcaactctaacgttctatagctctgctccgtctaaacaaggaattctgttaaatttgtaacctggactttcttgaatactgaggtaatgtttcttgaagtaggatatatatacccaagaagaataaaaataattctggaggtgtcttaattctctgtgggactcaataaaagttttggtgattatatataaacacacttatgaaagcatctggtacatgtaggtgctcagtgcacatgaatttctctttcctgccagatcttttgtagtggaaaattatcttattcttccatctttgtctgcaaagatgctgctaaggaaagatgtagaagagttttacaagggagttggagtatgggaacaggaagttcccaagaaggccactgcattaatagaattgaaacaagatcccctaaaggaaaatcgctgccaaatctcttttctctaaactatccaaaatggtgccccataaattttcattgacattgaagtacataatgtaataagcttttttttccttaaatatataatgtacggagaaaaccaggttgataatggtttcctggcatagctttcaaaggcaagtgtggatgaagtggaaatatggtgcacagatattggaaagaaaccgctgttgaactcttcacattttcatgtataacccaatgattctcaaacctaaacttgcatcagaattgcctggagggcttattaaaacatgattgttggatgccacccttgcattaattaattacacctgcaactgttctatttttaaatggtcacattttaaggtactgggctgaggacttcattatgtgagtttcaaatgggacataattcaacccctagcccaatcctagtgagtggtgagtggtatctcttcgttttgatttctacttccctagtgactaatgatgttgagcatttttatgtgtgtttattagccatttttatatctcttatgaaaaaatgtgtataaaat

SEQ ID NO: 62 - DNA, 5620 bp, Homo sapiens
  source 1..5620: mol_type = genomic DNA
gcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgagccagtcgctgtggttgttttagctccttgactccttgtggtttatgtcatcatacatgactcagcatacctgctggtgcagagctgaagattttggagggtcctccacaataaggtcaatgccagagacggaagcctttttccccaaagtcttaaaataacttatatcatcagcatacctttattgtgatctatcaatagtcaagaaaaattattgtataagattagaatgaaaattgtatgttaagttacttcactttaattctcatgtgatccttttatgttatttatatattggtaacatcctttctattgaaaaatcaccacaccaaacctctcttattagaacaggcaagtgaagaaaagtgaatgctcaagtttttcagaaagcattacatttccaaatgaatgaccttgttgcatgatgtatttttgtacccttcctacagatagtcaaaccataaacttcatggtcatgggtcatgttggtgaaaattattctgtaggatataagctacccacgtacttggtgctttaccccaacccttccaacagtgctgtgaggttggtattatttcattttttagatgagaaaatgggagctcagagaggttatatatttaagttggtgcaaaagtaattgcaagttttgccaccgaaaggaatggcaaaaccacaattatttttgaaccaacctaataatttaccgtaagtcctacatttagtatcaagctagagactgaatttgaactcaactctgtccaactccaaaattcatgtgctttttccttctaggcctttcataccaaactaatagtagtttatattctcttccaacaaatgcatattggattaaattgactagaatggaatctggaatatagttcttctggatggctccaaaacacatgtttttcttcccccgtcttcctcctcctcttcatgctcagtgttttatatatgtagtatacagttaaaatatacttgttgctggtactggcagcttatattttctctcttttttcatggattaaccttgcttgagggctttaacaattgtattactttttcaaagaactaagctttagcttcattgatttttttctatttaattgggttttgctcttctctttagcattggaaacatagaaatgctttctgatttctttgggtagatttacgtattcagcttcttgagatggaagtttagatcactgatccttcagcttgttttcttttttgtatacatagattttaggacgatatattttcccttgagttctgctttagctgcagctcttatgttttgatatgcctctctttattatccttcagttaaaaatatctttcaattcattgttatataaaaatatgtgcctagtttttaacatctggagattttctagttttgaaaaaaacataagccaggcatggtggctcacacctgtatccccagcactttgggaggccgagacgggaggatcgcctgagctcaggagtttttacaccagcctgggaataacagtgagacattatctccaaaaaaattacctgggtatggtgttgtgcacctgtagtcccagctactctggagactgaggtgggaggattgtttgagcttgggaggttgaggctgcagggagctgtgatcacaccactgcactctggcctgagtgacagattgagaccctgtctcaataaaagcaaaaataaagaaaataaaccatatgtgttgaacaaaggattaataaattaatttgagactccttcagggaatgaccacaatttattgaaaatagcctaaatgttggagtcaggcatttctggattcatattttgacatcatgctgtcatcttgaacaaaatgcctaacctttctgaacttcaacttccttgccactcaaataaggattacaaaacttaaaatgtggtaagtactaaagacgacagcaaaaattgagtccagcacagagcttcctaaataagcaagcactcaacagagttggttcctttcttcctcccctgcttgacaatccagtttcccacaggagcctttgtagctgtagccaccatggtcagtccagggattcttcactagccccttctcccctggcagacatccttgtgggagtttagtcttggctcgacatgaggatgggggtttgggaccagttctgagtgagaatcagacttgccccaagttgccattagctccccctgcagaatgtcttcagaatcggggcccggtcagtctcctgggtgacctgctgttttcctcttaagatcctttccactttggttgctgctttcgggactcatcgagtccttgctcaacaggataccccttgaagtggctgcctgggccacatccccttccaaacaagaaatcaaaatattagaaatcaatttttgaaatttcccctaggaagactcatttgagtgttcaagttcagagccagtggagaccttaggggagggtggtcacaaggattttgcacagtgctttagagggtcccagggagccacagaggtggtgaggggctgggtgctcttttctccgtgcatgaccttgtgtgtctatcttcattaccacaatgcctcatctctacctcctttccccctgtagttccaacgtgggtatctttgccatctctggcccgaaggactttctgacctacatgtataaataccccctcacaatatatattacttttcctataagtgacttctctactggattactggttgctcatacacctcatattttactcgtaaatctactactccctgtctgcctactccattctcatttgctgtagaaaattctcttaccatcccaactttcacccaccatcatgcttacccaaaggctgtgggaatgacctgggccctaatgccccttttctaaattcctaaggctcaccattttcctattgtaatggttcttgaccttataatgtttgaggcaccttttcaaatatagtcctttgatttcagactgaatacttgaaaggacacacacacacatacgtaagtgcatatgactgcatacacccacacacacacacgtgcctgtatacagtcatatgatacatacacaaacacacgcacacaagcctgcatacatcatatgccaacagtggggatatgttctgagaaatgcatcattagatgattttgtcattgtgtgaacatcatagagtgtacttacactaacctagatggtctaacctactacacacccaggctacatggtatcacctattcctcctaggctacaagcctgtacagcgtgtgtctgtactaaatgctgtgggcaattttaacctgatggtaaatgtttgtgtatctaaacatatctaaacatagaaaaggtacagtaaacatgcagtattataatcttatgagaccgtcatcatatatgtggtccactgtttgggccatcattggctgaaaagtggttatgcgacacatgactgtatatatactttcctgttacaacaacagtgtctctcaatccacagtaattgcagcatccagtaggtcttactttagccctgagtcaccatttgtgtcaacgtgtttagtgccatgtccacgtctctcatgtaactggcagagctatcaaatattttggcaaaacacattgtttctttggctttgccttggtaactttctgtgccttttgtagctcttgtttggaagaagctcaacccatgtctgcacactgtgatacaagggggacagcatcgacatcgacttacttcttggtgccttattcctccttagaacaattcctaaatctgtaacttaagtttctcaggaagattccatactgcacagaaaactgcttttgtgggtttttaaaaggcaagttgttatatgtgctggatagtttttaagtatgacataaaaattgtataaagtaaaatattaaaatacacctagaatactgtataactttaagtcattttatcaacacattgctaatccagatattttcccgcagtttttctttgaataacagagcaattaatttacttttactatgaagagtcatcattttagtatgtattttaagcaatccaccaagaactcagtaggcagctgagaggtgctgcccagagaagtggtgattagcttggccttagctcacccacacaaagcacaacaggctttgaactattccctaacggggcatttattcttttttttttttttttttgggagacggagtctcgctgtcgcccaggctagagtgcagtggcgcgatctcggctcactgcaggctccaccccctggggttcacgccattctcctgcctcagcctcccaagtagctgggactgcaggcgcccgccatctcgcccggctaattttttgtatttttagtagagacggggtttcaccgtgttagccaggatagggcatttattcttgaacttgattcagagaggcacacattaccattctctaatcagaatgcaagtagcgcaaggcggtggaaactatggaattcggaggcaggtgatgcattgggcgagtttattaacatctgtgactctctagtttgaaatttatttgtaacagacaaaaatgaattaaacaaacaataaaagtataataaagaa

SEQ ID NO: 63 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 64 - DNA, 23 bp, Homo sapiens
  source 1..23: mol_type = genomic DNA
ctggacccatgtgcaccccaagg

SEQ ID NO: 65 - DNA, 23 bp, Homo sapiens
  source 1..23: mol_type = genomic DNA
ccttggggtgcacatgggtccag

SEQ ID NO: 66 - RNA, 23 nt, Homo sapiens
  source 1..23: mol_type = genomic RNA
ctggacccatgtgcaccccaagg

SEQ ID NO: 67 - DNA, 23 bp, Homo sapiens
  source 1..23: mol_type = genomic DNA
tggacccatgtgcaccccaaggg

SEQ ID NO: 68 - DNA, 23 bp, Homo sapiens
  source 1..23: mol_type = genomic DNA
cccttggggtgcacatgggtcca

SEQ ID NO: 69 - RNA, 23 nt, Homo sapiens
  source 1..23: mol_type = genomic RNA
tggacccatgtgcaccccaaggg

SEQ ID NO: 70 - DNA, 23 bp, Homo sapiens
  source 1..23: mol_type = genomic DNA
ggacccatgtgcaccccaagggg

SEQ ID NO: 71 - DNA, 23 bp, Homo sapiens
  source 1..23: mol_type = genomic DNA
ccccttggggtgcacatgggtcc

SEQ ID NO: 72 - RNA, 23 nt, Homo sapiens
  source 1..23: mol_type = genomic RNA
ggacccatgtgcaccccaagggg

SEQ ID NO: 73 - DNA, 23 bp, Homo sapiens
  source 1..23: mol_type = genomic DNA
ccaaggggcctaagtcattgctg

SEQ ID NO: 74 - DNA, 23 bp, Homo sapiens
  source 1..23: mol_type = genomic DNA
cagcaatgacttaggccccttgg

SEQ ID NO: 75 - RNA, 23 nt, Homo sapiens
  source 1..23: mol_type = genomic RNA
ccaaggggcctaagtcattgctg

SEQ ID NO: 76 - DNA, 23 bp, Homo sapiens
  source 1..23: mol_type = genomic DNA
cccaaggggcctaagtcattgct

SEQ ID NO: 77 - DNA, 23 bp, Homo sapiens
  source 1..23: mol_type = genomic DNA
agcaatgacttaggccccttggg

SEQ ID NO: 78 - RNA, 23 nt, Homo sapiens
  source 1..23: mol_type = genomic RNA
cccaaggggcctaagtcattgct

SEQ ID NO: 79 - DNA, 23 bp, Homo sapiens
  source 1..23: mol_type = genomic DNA
ccccaaggggcctaagtcattgc

SEQ ID NO: 80 - DNA, 23 bp, Homo sapiens
  source 1..23: mol_type = genomic DNA
gcaatgacttaggccccttgggg

SEQ ID NO: 81 - RNA, 23 nt, Homo sapiens
  source 1..23: mol_type = genomic RNA
ccccaaggggcctaagtcattgc

SEQ ID NO: 82 - DNA, 23 bp, Homo sapiens
  source 1..23: mol_type = genomic DNA
ccatgtgcaccccaaggggccta

SEQ ID NO: 83 - DNA, 23 bp, Homo sapiens
  source 1..23: mol_type = genomic DNA
taggccccttggggtgcacatgg

SEQ ID NO: 84 - RNA, 23 nt, Homo sapiens
  source 1..23: mol_type = genomic RNA
ccatgtgcaccccaaggggccta

SEQ ID NO: 85 - DNA, 7613 bp, Homo sapiens
  source 1..7613: mol_type = genomic DNA
aaacagaaatgaacaaataaacacacaagatcatttcccgtggtagtgagagctgggatgaaaataaaacagcgtggcagggaggaggcaagtgttgtgagtctggagggttcctggagaatggggcctgaggcgtgaccaccgccttcctctctggggggactgcctgccgcccccgcagacacccatggttgagtgccctccaggcccctgcctgccccagcatcccctgcgcgaagctgggtgccccggagagtctgaccaccatgccacctcctcgcctcctcttcttcctcctcttcctcacccccatggaagtcaggcccgaggaacctctagtggtgaaggtggaaggtatgtccaaagggcagaaagggaagggattgaggctggaaacttgagttgtggctgggtgtccttggctgagtaacttaccctctctgagcctccattttcttatttgtaaaattcaggaaagggttggaaggactctgccggctcctccactcccagcttttggagtcctctgctctataacctggtgtgaggagtcggggggcttggaggtcccccccacccatgcccacacctctctccctctctctccacagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggtgagggccgggctggggcaggggcaggaggagagaagggaggccaccatggacagaagaggtccgcggccacaatggagctggagagaggggctggagggattgagggcgaaactcggagctaggtgggcagactcctggggcttcgtggcttcagtatgagctgcttcctgtccctctacctctcactgtcttctctctctctgcgggtctttgtctctatttatctctgtctttgagtctctatctctctccctctcctgggtgtctctgcatttggttctgggtctcttcccaggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccagggtatggtgatgactggggagatgccgggaagcgggggtccagagacagaggggaggggaaactgaagaggtgaaaccctgaggatcaggctttccttgtcttatctctccctgtcccagacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccaggtagagtttctctcaactgggaggcatctgtgtgggggtactgggaagaagtggaagccagtcaatcttagattcccccaacccgagggctactcccagcctcaccccaaaccccaacttccacacagaacactgactccaagtctttcttttttttgacagagtctcgctctgttgcctaggctggagtgcagtggtgccatcttgtcttggctcactgcaacctccgcctcccaggttcaagtgattcccctgcctcagcctcctgagtagctgggattacaggtgcccaccaccacgcctggctaattttttttttttttttgagacggagtcttgcactgtcacccaggctggagtgcagtggcacgatctcagctcactgcaacctccaccttccaggttcaagtgattctcctgcctcagcctcccgagtagctgggattaaagcctggctaattttttttgtatttttagtagagatggggtttcattatgttggccaggctggtctcaaactcctgacctcgtgatccacccgcctcggcctcccaaagtgctgggattacagacatgagccacagggccgggccaagcctaattttgtatttttagtagagatggggtttctccctgttggaccaggctggtcttgaactcctgacttcaggtgatctgcctgccttggcctcccaaagtactgggattacaggcataagccaccgcacctggcctagacttcaagtctttcttccctcgcttccaagacactacttttctgggtcttcacctaccattgcttgcgcctgcccaccagcttgggtggagtcttccttcctccccaactcctcactcttggagccctgggccctcttcttatccctgtctgcacactttcctatttgaacttgactctcaatggcttcttgggtcaccatgccttggtgactctattccaggctccatactcagccatctcctgtgccatttgatatcccatggacacctcaggctcaacagatacaaaatcaaactcaatgtcttccccaagtatagtcttcttggtggcccagtgtaagcagagggcaccaccacctgctccctcgcccaggctaagaacctgggcatccttctttttcctcaccccgtccaacaaactggtcacagtgttctgccaattctctctccatgcaatcctatcatgctatcctaactgcaattcacaaacccaaccccaactttcactccaaacttgatccaagcaatgtgctggatcccaactgtaaccttgcaaactcaactctgcccttcactttgaccgtgactatccttaattgcagcaggaaactgatcattatgctcccctcaatccacacattgcctctgagtacagccatggtttgtccacgatttgctcaaagacactgccccatgtcctgtgccagggtctgtgacaatccctgacctcctgggacatggctccttagagagaggagagcctttctcacagcttgggactttgagtctgtgtctttttttttttcttgagacggagttttgctgtggttgcccaggctggagtgcagtgatctcggctcactgaaacctccgcctcccgggttcaaacgattctcctgcctcagcctcccaagtagctgggattacaggcacccaccaccatgcccagctaatttttttgtatttttagtagagatggggtttcaccatgttggccaggctggtctcgaactcctgacctcaggtgatccacccgcctttgcctcccaaagtgctgggattacaggcgtcaaccaccgcgcccggccgagtctgtgtcttgcctctgtgcctcagacttgcggttccttgagatctcaggattgggacgtaagatgccagcctggggtcctcgtctcatagccccttccccctagtactatggcactggctgctgaggactggtggctggaaggtctcagctgtgactttggcttatctgatcttctgcctgtgttcccttgtgggcattcttcatcttcaaagaggtgagtcatgtccccagtgggtctgtccaaaccctactccatcttccccaggataagccggctctggccagtctgacaaccatctttctttcctcccatccctcccttcaagaccccagaatcctgttctccccagtcttcctctagcctccctcaaacttcccaagcctcttgcaattttttttttttttttgagacagggtctcattctgtcaccccagctggagtgcagtggcacaatctgagctcactgtaacctctgcctcccaggcttaagtgattcttgtgcttcagcctcccgagtacctgggactacaagtgtatgccaccacacccggccaattttttatatttttagtagagacgaggtttcaccatgttggccagactggtctcgaactcttgacctcaaatgatccgcccacctcggcctcccaaagtgctgggattacaggcacgagccaccgcgcccgtccgcctcgcaatttgaactcctgtctcctttgttgaaccaagtgacctccccagcacctggccccacaaatcctcaccctgccaagcagcccctcctctgatcacgccctttaactcccaccagccctggtcctgaggaggaaaagaaagcgaatgactgaccccaccaggaggtaatgcaaccagtgcaccccgcggtaacaccctccaccttcactttatgccttgcacttactgtttcctctgcccaggggttctttgctccgtctctactgtttcaaatactgcccaacctcaaagcccagctccaaagctacctcctctgtgaagaactccttggaaatgatcatctcagactcctctattggctgtcccagcacaagtgatcacgtttaacttctgaaggcctggacagaatcttgagtgggtccgccattccattccaagtcggccctcaccgtgcacttcctcttctcccgccagattcttcaaagtgacgcctcccccaggaagcgggccccagaaccagtacgggaacgtgctgtctctccccacacccacctcaggcctcggtaagaggcaccgcccctccagcctatagctccgccccagatccggggctccacccccactctcctcatccctccaatccgctgtgcgccaagccttctggagctcggaactccgcccccggggcggggagtcccgcccagctatgagccccgcctctagaaccagaccccgcctccagggctcagagccacgcccccaggacccagagcctgaagtcgtaatcaagagcagaacttcgccccagaactgaaggcctcggccctagatttagattccgccccagggttcaaggccgggttcctagacccagagtccattcgcagagcccaaaacatcctcttcccgtgccccgccgcgcggacccttagccttgaccgcccccatctcttctgaccccgtcttacaatgcccctctcaccaggacgcgcccagcgttgggccgcaggcctggggggcactgccccgtcttatggaaacccgagcagcgacgtccaggcggatggagccttggggtcccggagcccgccgggagtgggtgaatgactgggagagggaagggtcgttccccacatggagggggttggagcggtctgtggcccgaatagtggactgggccctggaggagagggggcatgactcggttccccatccccatccccaaacccccaggcccagaagaagaggaaggggagggctatgaggaacctgacagtgaggaggactccgagttctatgagaacgactccaaccttgggcaggaccagctctcccagggtaaggctgccctcccccgtggccccccacctctgcggtggcctgtggactcccatggacacccctccttctccaccagatggcagcggctacgagaaccctgaggatgagcccctgggtcctgaggatgaagactccttctccaacggtaacttggggcctttgtgggacctcagagacttaggtgtaattgcagcgctgtgacactcctagaaggggtccctggagttctctctcttctgccacagctgagtcttatgagaacgaggatgaagagctgacccagccggtcgccaggacaatgggtgtgtgtgaggatggcaacagtccaggggggaggcggaggacacctggaggccaggaggaatagtaacctccctcttccctttccagacttcctgagccctcatgggtcagcctgggaccccagccgggaagcaacctccctgggtgagagatgctttcaatcagactgccttgcccagcttgggtgacctggcctcagctctgacaccagatccaactttgacctgaccctgaccccaaacccgaacccaatcctgtgactcctctcacctcaacactgagccccatcccccatcctgagccccatcccccatcctgacccccaatatttaccccctccctaactgtgaatatcaacaccgatcccaatgcagtatcagcctggacttgatctccacctcacctcagccccagtgcagacctcaacttggaccccagcttactctgcagcttcttcatgactctgactccgactccctccagtttcttctttttctttttcttttttttgagacggagtctccctctgttgcccaggctggagtgcagttgccacctctgcctcctaggttcaagcgattctcatgcctcagcctcctgagtagctgggattatagacgtttgccaccacacctggctaatttttgtattttcagtagagacagggtttcgccatgttggccagactggtctccaactcctggcctctagtgatctgcccgccttggcttcccaaagtgctgggattacaggcatgagccaccacgcccagcccagttctgttcttgaccccttccttagccataatctaacccatatctaaccctgaccctacagctaactggggccccaaactcaatgctaaccaaatcaccccttcccagcacagcatgggtaatgctcctcaccttcctctgcccctcagtcttcctccttaccgtaggctgtacttcccatgccctagcctccaattctccatcccccgcccaagcagggtcccagtcctatgaggatatgagaggaatcctgtatgcagccccccagctccgctccattcggggccagcctggacccaatcatgaggaaggtgggtgcttctgccgctgtcccctgctgtcccctgggctgactttgccttccagcctacttccagtgccacccatgttctcctcctccctggtcctatccagatgcagactcttatgagaacatggataatcccgatgggccagacccagcctggggaggagggggccgcatgggcacctggagcaccaggtgatcctcaggtggccaggtgagctgggactgcccctagggaaagcggggagggagggagataggcacggatggcagtggctgctggctttcagggagggagagggaacagggttcctagggcctggtgggcagggggaggactgctggacccctccccatcaccgtttcttctgcatagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaatccta

SEQ ID NO: 86 - DNA, 1918 bp, Homo sapiens
  source 1..1918: mol_type = genomic DNA
aagctgggtgccccggagagtctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgatcctcaggtggccagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaa

SEQ ID NO: 87 - protein, 556 aa, Homo sapiens [sequence omitted]

SEQ ID NO: 88 - protein, 107 aa, Synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 89 - protein, 24 aa, Synthetic construct
IYIWAPLAGTCGVLLLSLVITLYC

SEQ ID NO: 90 - protein, 68 aa, Synthetic construct
FWVLVVVGGVLACYSLLVTVAFIIFWVRSKRSRLLHSDYMNMTPRRPGPTRKHYQPYAPPRDFAAYRS

SEQ ID NO: 91 - protein, 41 aa, Synthetic construct
KRGRKKLLYIFKQPFMRVQTTQEEDGCSCRFPEEEEGGCEL

SEQ ID NO: 92 - protein, 113 aa, Synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 93 - DNA, 200 bp, Synthetic construct
  source 1..200: mol_type = other DNA
aataaaataaaaataaaaaaacaatagagagtatgatttaccggcatagaatagtcggcgtctttgacgcacttaatatcggtcacgttcctattgagatcccaggtcaactgctgagcctttgctttcatccttaggttcgtgattggtgggtttggatctaaaacggtgacaggttggagttcgaagagatacgtaag

SEQ ID NO: 94 - DNA, 200 bp, Synthetic construct
  source 1..200: mol_type = other DNA
aaataaatacataaatacataaataaataaaataaaaataaaaaaacaatagagagtatgatttaccggcatagaatagtcggcgtctttgacgcacttaatatcggtcacatttctgttaaggtcccaggtcaactgctgagcctttgctttcatccttaggttcgtgattggtgggtttggatctaaaacggtgacag

SEQ ID NO: 95 - DNA, 180 bp, Synthetic construct
  source 1..180: mol_type = other DNA
ataaatacataaataaataaaataaaaataaaaaaacaatagagagtatgatttaccggcatagaatagtcggcgtctttgacgcacttaatatcggtcacatttctgttaaggtcccaggtcaactgctgagcctttgctttcatccttaggttcgtgattggtgggtttggatctaaa

SEQ ID NO: 96 - DNA, 150 bp, Synthetic construct
  source 1..150: mol_type = other DNA
aataaaataaaaataaaaaaacaatagagagtatgatttaccggcatagaatagtcggcgtctttgacgcacttaatatcggtcacatttctgttaaggtcccaggtcaactgctgagcctttgctttcatccttaggttcgtgattggt

SEQ ID NO: 97 - DNA, 120 bp, Synthetic construct
  source 1..120: mol_type = other DNA
aaaaaacaatagagagtatgatttaccggcatagaatagtcggcgtctttgacgcacttaatatcggtcacatttctgttaaggtcccaggtcaactgctgagcctttgctttcatcctt

SEQ ID NO: 98 - DNA, 100 bp, Synthetic construct
  source 1..100: mol_type = other DNA
agagagtatgatttaccggcatagaatagtcggcgtctttgacgcacttaatatcggtcacatttctgttaaggtcccaggtcaactgctgagcctttgc

SEQ ID NO: 99 - DNA, 80 bp, Synthetic construct
  source 1..80: mol_type = other DNA
atttaccggcatagaatagtcggcgtctttgacgcacttaatatcggtcacatttctgttaaggtcccaggtcaactgct

SEQ ID NO: 100 - DNA, 20 bp, Homo sapiens
  source 1..20: mol_type = genomic DNA
accgatatcgagtgtgttaa

SEQ ID NO: 101 - DNA, 20 bp, Homo sapiens
  source 1..20: mol_type = genomic DNA
gatatcgagtgtgttaaaga

SEQ ID NO: 102 - DNA, 20 bp, Homo sapiens
  source 1..20: mol_type = genomic DNA
atcgagtgtgttaaagacgc

SEQ ID NO: 103 - DNA, 20 bp, Homo sapiens
  source 1..20: mol_type = genomic DNA
aatgtgaccgatatcgagtg

SEQ ID NO: 104 - DNA, 20 bp, Homo sapiens
  source 1..20: mol_type = genomic DNA
ctcgatatcggtcacatttc

SEQ ID NO: 105 - DNA, 20 bp, Homo sapiens
  source 1..20: mol_type = genomic DNA
atattcaattttcctgcaag

SEQ ID NO: 106 - DNA, 20 bp, Homo sapiens
  source 1..20: mol_type = genomic DNA
attcaattttcctgcaagaa

SEQ ID NO: 107 - DNA, 20 bp, Homo sapiens
  source 1..20: mol_type = genomic DNA
ttcccgcagggtaagtacca

SEQ ID NO: 108 - DNA, 20 bp, Homo sapiens
  source 1..20: mol_type = genomic DNA
cccgcagggtaagtaccaag

SEQ ID NO: 109 - DNA, 20 bp, Homo sapiens
  source 1..20: mol_type = genomic DNA
ccgcagggtaagtaccaagt

SEQ ID NO: 110 - DNA, 20 bp, Homo sapiens
  source 1..20: mol_type = genomic DNA
gcagggtaagtaccaagtag

SEQ ID NO: 111 - DNA, 20 bp, Homo sapiens
  source 1..20: mol_type = genomic DNA
tgggaactcagaccgtacct

SEQ ID NO: 112 - DNA, 20 bp, Homo sapiens
  source 1..20: mol_type = genomic DNA
ggaactcagaccgtaccttg

SEQ ID NO: 113 - DNA, 20 bp, Homo sapiens
  source 1..20: mol_type = genomic DNA
gaactcagaccgtaccttgc

SEQ ID NO: 114 - DNA, 20 bp, Homo sapiens
  source 1..20: mol_type = genomic DNA
actcagaccgtaccttgcaa

SEQ ID NO: 115 - DNA, 20 bp, Homo sapiens
  source 1..20: mol_type = genomic DNA
ctcagaccgtaccttgcaac

SEQ ID NO: 116 - DNA, 20 bp, Homo sapiens
  source 1..20: mol_type = genomic DNA
cagaccgtaccttgcaacaa

SEQ ID NO: 117 - DNA, 20 bp, Homo sapiens
  source 1..20: mol_type = genomic DNA
caaagaccgccctgagatct

SEQ ID NO: 118 - DNA, 20 bp, Homo sapiens
  source 1..20: mol_type = genomic DNA
agaccgccctgagatctggg

SEQ ID NO: 119 - DNA, 20 bp, Homo sapiens
  source 1..20: mol_type = genomic DNA
gaccgccctgagatctggga

SEQ ID NO: 120 - DNA, 20 bp, Homo sapiens
  source 1..20: mol_type = genomic DNA
tccctgggtaggtaactctg

SEQ ID NO: 121 - DNA, 20 bp, Homo sapiens
  source 1..20: mol_type = genomic DNA
cagagttacctacccaggga

SEQ ID NO: 122 - RNA, 20 nt, Homo sapiens
  source 1..20: mol_type = genomic RNA
tccctgggtaggtaactctg

SEQ ID NO: 123 - DNA, 20 bp, Homo sapiens
  source 1..20: mol_type = genomic DNA
ttccctgggtaggtaactct

SEQ ID NO: 124 - DNA, 20 bp, Homo sapiens
  source 1..20: mol_type = genomic DNA
agagttacctacccagggaa

SEQ ID NO: 125 - RNA, 20 nt, Homo sapiens
  source 1..20: mol_type = genomic RNA
ttccctgggtaggtaactct

SEQ ID NO: 126 - DNA, 20 bp, Homo sapiens
  source 1..20: mol_type = genomic DNA
gttccctgggtaggtaactc

SEQ ID NO: 127 - DNA, 20 bp, Homo sapiens
  source 1..20: mol_type = genomic DNA
gagttacctacccagggaac

SEQ ID NO: 128 - RNA, 20 nt, Homo sapiens
  source 1..20: mol_type = genomic RNA
gttccctgggtaggtaactc

SEQ ID NO: 129 - DNA, 20 bp, Homo sapiens
  source 1..20: mol_type = genomic DNA
tttgaaaatgttccctgggt

SEQ ID NO: 130 - DNA, 20 bp, Homo sapiens
  source 1..20: mol_type = genomic DNA
acccagggaacattttcaaa

SEQ ID NO: 131 - RNA, 20 nt, Homo sapiens
  source 1..20: mol_type = genomic RNA
tttgaaaatgttccctgggt

SEQ ID NO: 132 - DNA, 20 bp, Homo sapiens
  source 1..20: mol_type = genomic DNA
aacatttgaaaatgttccct

SEQ ID NO: 133 - DNA, 20 bp, Homo sapiens
  source 1..20: mol_type = genomic DNA
agggaacattttcaaatgtt

SEQ ID NO: 134 - RNA, 20 nt, Homo sapiens
  source 1..20: mol_type = genomic RNA
aacatttgaaaatgttccct

SEQ ID NO: 135 - DNA, 20 bp, Homo sapiens
  source 1..20: mol_type = genomic DNA
aaacatttgaaaatgttccc

SEQ ID NO: 136 - DNA, 20 bp, Homo sapiens
  source 1..20: mol_type = genomic DNA
gggaacattttcaaatgttt

SEQ ID NO: 137 - RNA, 20 nt, Homo sapiens
  source 1..20: mol_type = genomic RNA
aaacatttgaaaatgttccc

SEQ ID NO: 138 - DNA, 20 bp, Homo sapiens
  source 1..20: mol_type = genomic DNA
accccagagttacctaccca

SEQ ID NO: 139 - DNA, 20 bp, Homo sapiens
  source 1..20: mol_type = genomic DNA
tgggtaggtaactctggggt

SEQ ID NO: 140 - RNA, 20 nt, Homo sapiens
  source 1..20: mol_type = genomic RNA
accccagagttacctaccca

SEQ ID NO: 141 - DNA, 20 bp, Homo sapiens
  source 1..20: mol_type = genomic DNA
tggaacgggtgagccttgcc

SEQ ID NO: 142 - DNA, 20 bp, Homo sapiens
  source 1..20: mol_type = genomic DNA
ctggcacttcgagttggaac

SEQ ID NO: 143 - DNA, 20 bp, Homo sapiens
  source 1..20: mol_type = genomic DNA
ccgttccaactcgaagtgcc

SEQ ID NO: 144 - DNA, 20 bp, Homo sapiens
  source 1..20: mol_type = genomic DNA
cctggcacttcgagttggaa

SEQ ID NO: 145 - DNA, 20 bp, Homo sapiens
  source 1..20: mol_type = genomic DNA
cgttccaactcgaagtgcca

SEQ ID NO: 146 - DNA, 20 bp, Homo sapiens
  source 1..20: mol_type = genomic DNA
ctcgaagtgccagggccagc

SEQ ID NO: 147 - DNA, 20 bp, Homo sapiens
  source 1..20: mol_type = genomic DNA
gaagtgccagggccagctgg

SEQ ID NO: 148 - DNA, 20 bp, Homo sapiens
  source 1..20: mol_type = genomic DNA
ggtagacctccagctggccc

SEQ ID NO: 149 - DNA, 20 bp, Homo sapiens
  source 1..20: mol_type = genomic DNA
ccagctggaggtctacctca

SEQ ID NO: 150 - DNA, 20 bp, Homo sapiens
  source 1..20: mol_type = genomic DNA
ccttgaggtagacctccagc

SEQ ID NO: 151 - DNA, 20 bp, Homo sapiens
  source 1..20: mol_type = genomic DNA
ctggaggtctacctcaagga

SEQ ID NO: 152 - DNA, 20 bp, Homo sapiens
  source 1..20: mol_type = genomic DNA
aggtctacctcaaggacgga

SEQ ID NO: 153 - DNA, 20 bp, Homo sapiens
  source 1..20: mol_type = genomic DNA
ccatgtgccatccgtccttg

SEQ ID NO: 154 - DNA, 20 bp, Homo sapiens
  source 1..20: mol_type = genomic DNA
cctcaaggacggatggcaca

SEQ ID NO: 155 - DNA, 20 bp, Homo sapiens
  source 1..20: mol_type = genomic DNA
acatggtttgcagccagagc

SEQ ID NO: 156 - DNA, 20 bp, Homo sapiens
  source 1..20: mol_type = genomic DNA
tttgcagccagagctggggc

SEQ ID NO: 157 - DNA, 20 bp, Homo sapiens
  source 1..20: mol_type = genomic DNA
tggagctccggccccagctc

SEQ ID NO: 158 - DNA, 20 bp, Homo sapiens
  source 1..20: mol_type = genomic DNA
gggccggagctccaagcagt

SEQ ID NO: 159 - DNA, 20 bp, Homo sapiens
  source 1..20: mol_type = genomic DNA
ccggagctccaagcagtggg

SEQ ID NO: 160 - DNA, 20 bp, Homo sapiens
  source 1..20: mol_type = genomic DNA
cctcccactgcttggagctc

SEQ ID NO: 161 - DNA, 20 bp, Homo sapiens
  source 1..20: mol_type = genomic DNA
agacttttgacgcttgactg

SEQ ID NO: 162 - DNA, 20 bp, Homo sapiens
  source 1..20: mol_type = genomic DNA
cagacttttgacgcttgact

SEQ ID NO: 163 - DNA, 20 bp, Homo sapiens
  source 1..20: mol_type = genomic DNA
gcagacttttgacgcttgac

SEQ ID NO: 164 - DNA, 20 bp, Homo sapiens
  source 1..20: mol_type = genomic DNA
gtctgccagcggctgaactg

SEQ ID NO: 165 - DNA, 20 bp, Homo sapiens
  source 1..20: mol_type = genomic DNA
tctgccagcggctgaactgt

SEQ ID NO: 166 - DNA, 20 bp, Homo sapiens
  source 1..20: mol_type = genomic DNA
ctgccagcggctgaactgtg

SEQ ID NO: 167 - DNA, 20 bp, Homo sapiens
  source 1..20: mol_type = genomic DNA
gcaccccacagttcagccgc

SEQ ID NO: 168 - DNA, 20 bp, Homo sapiens
  source 1..20: mol_type = genomic DNA
aggaaggggccaaggcttaa

SEQ ID NO: 169 - DNA, 20 bp, Homo sapiens
  source 1..20: mol_type = genomic DNA
aaggaaggggccaaggctta

SEQ ID NO: 170 - DNA, 20 bp, Homo sapiens
  source 1..20: mol_type = genomic DNA
aggtgacaaggaaggggcca

SEQ ID NO: 171 - DNA, 20 bp, Homo sapiens
  source 1..20: mol_type = genomic DNA
gtgtgtaggtgacaaggaag

SEQ ID NO: 172 - DNA, 20 bp, Homo sapiens
  source 1..20: mol_type = genomic DNA
ggtgtgtaggtgacaaggaa

SEQ ID NO: 173 - DNA, 20 bp, Homo sapiens
  source 1..20: mol_type = genomic DNA
gattgagctctgaggtgtgt

SEQ ID NO: 174 - DNA, 20 bp, Homo sapiens
  source 1..20: mol_type = genomic DNA
tagcagatgattgagctctg

SEQ ID NO: 175 - DNA, 20 bp, Homo sapiens
  source 1..20: mol_type = genomic DNA
cagagctcaatcatctgcta

SEQ ID NO: 176 - DNA, 20 bp, Homo sapiens
  source 1..20: mol_type = genomic DNA
aatcatctgctacggacaac

SEQ ID NO: 177 - DNA, 20 bp, Homo sapiens
  source 1..20: mol_type = genomic DNA
atcatctgctacggacaact

SEQ ID NO: 178 - DNA, 20 bp, Homo sapiens
  source 1..20: mol_type = genomic DNA
gacacatgtcatttctgctg

SEQ ID NO: 179 - DNA, 20 bp, Homo sapiens
  source 1..20: mol_type = genomic DNA
aaatgacatgtgtcactctc

SEQ ID NO: 180 - DNA, 20 bp, Homo sapiens
  source 1..20: mol_type = genomic DNA
aatgacatgtgtcactctct

SEQ ID NO: 181 - DNA, 20 bp, Homo sapiens
  source 1..20: mol_type = genomic DNA
gttacccacctaagcaggtc

SEQ ID NO: 182 - DNA, 32306 bp, Homo sapiens
  source 1..32306: mol_type = genomic DNA
gagtcttgctgatgctcccggctgaataaaccccttccttctttaacttggtgtctgaggggttttgtctgtggcttgtcctgctacatttcttggttccctgaccaggaagcaaagtgattaacggacagttgaggcagccccttaggcagcttaggcctgccttgtggagcatccccgcggggaactctggccagcttgagcgacacggatcctcagagcgctcccaggtaggcaattgccccagtggaatgcctcgtcagagcagtgcatggcaggcccctgtggaggatcaacgcagtggctgaacacagggaaggaactggcacttggagtccggacaactgaaacttggtaagactagtccttggaacttgcccactccatttgagtggaagcatggcccaatcacccatggcatgcctttatcagcactttggttttggttttggttttgacttggtttgaatcgcttgacaggactggtcttgggaacttgcctactccatttgagtagaagcgtgtcctgatcacccacagtgtgcctgtaccagcactttggtttttgtttttgacttgtcttggattgcttgatactttggttttggttttgacctggcttggatttcttgatactctgattttggttttgattctggcttggcgtaaactgtaaaagtgtgtgtgtgccctttttacccgttctttgttttgtggtgtgagcatggtgttttgtctcgaggaaacatgggtcagacacaaagtaagcctaccccactaggaactatgttgaaaaatttcaaaaagggatttaagggagactctggagtcactatgacaccaggaaaacttagagctttgtgtgagatagactggccagcattagaggtgggttggccatcggaaggaagcctggacaggtcccttgtctcgaaggtatggcacagggtaacctgtaagccagggcacccagatcagttctcacatatagattcttggttacagctagttttggaccccccgcagtggttaagaggacaggcagcagcagtactagtagcaaagggacagttagttaaggaaggttctcgatcccacccgccgagggaagtcagcaccaaaagtcctgtccaacccaacaccagaagagtcacagcaggaattggtaccagcagtatcccctccttatcaagaggaagggctccccactcctgagcccacagcacctacaactccaccagataaccacaccccagaccacccagagtagacaaaagaggaagtgaagccacgggagaaactcctcccttgaaagctcgcttacagcccaagactggaatccaaatgcccctgagagagcagtgatatactggggtagatgaggacggacacatggtggaaaggcgtgcctttgtgtatcaaccttttacctctgctgacctcctcaattggaaaaataatactccatcttacactgaaaagcctcaagctttaattgacttgctccaaactattatacagactcataatcctacttgggctgactgccaccagctgctcatgtacctctttaatacaggtgaaaggtgaagggtgctccaggcggcaactaagtggctagagaagcacgtcccagccaattaccaaaacccccaagaatatataagaattcagctgccaggaacagacccccaataggacctgaacgagggaccagacatggagaggctaagacggtaccgtgaggcattaatagaaggtctaaggaaaggggctcaaaaggctacaaatgtaaataaggtctctgaggtcatccaaggaaaagaggagagtccagcacaattctatgaaagactgtgtgaggcttactgtatgtacactccttttgatccagatagccctgagaatcagcccatgattaacatggccttacttagtcaaagcacggaatatatcaggagaaaattgcagaaacaggctgggtttgcgggtatgaatacctcacagttactggggtttgcgggtatgaatacctcacagttactggaaatagtcaatcaagtgtttgagaatgcaacaagccacagagaaagccgtaaggaaggcgaacgccaggctaggcgaaacgccgacttactggccatggccattatgggaattcccccgaaaggagagggaaaggggggttctgggaagaatacccagtctaatctcccatgcttgcaatgtaaccaatgcgcctgttgtaaggaagtagggcattggaaagataagtgtccccaactgaaggaaaagcaaggtgattcagaacaaaagacctcagataaagatgagggagctttgttcaatctggctgaagggctactggactgaaggggaccgggcgcaagcacccccaaggagcccaaggtcaggattacaactgggggcaaggacgttaagtttttggttgatagtggtgctgaaccttcagtagtgaccaccccggtcaccccctcatccaagaaaaccattgatataatcagagcaacaggagtttccactaagcaggctttctgtctaccacggacctgctcggtggggggacatgagatagctcaccagttcttgtacatgcctgactgtcccttgcccttgctgggaagagacttgcttagcaagttgagaaccaccatctcctttacaaagcagggctctttacagctaaagttaccagcaacaggagttatcatggcccttacggtcccccgggaagaagaatgaagactttttctaaccaagccaggccaagagataaaaccagctctagctaagcaatggccccaagtatggacagaggataatcctccaggactggtggtcagccaagcccctgtactcatagaagttaagcctggggcccaaccaattagacaaaagcagtatctggttcccagagaagctctcgaaggaatccaggttcctctcaggcgcttgacagcctatggaattatagttccttgccagtctccacggaacacccccctcctacctgtccctaaaccagggaccaaggactactggccagtacaggacttggtccgaggagaagtttacaaaccctctcctgagccagtgtttcaaactttctaagatgaactcaatgtgccagtaccacaaattccaggaaaaacaagaaatttgtttttgcaattagccgagcacgtagcccagtctctcaatgtcacttcatgttatgtatgcggaggaactgtaatgggaggtcaatggccatgggaagcccaagaattactacctacagacccagttcctgatgaattcttggctcaaaagaatcaccctgataatttctgggtcccaaaagcctcaattattggacaatattgcatagctagagaaggaaaagaattcactcaccccgtaggacgacttagttgtctgggacagaaactgtataatggtaccacagaaacagtcacttggtggagttcaaatcacatagagaggaatccatttagtaaattcccaaagttgcaaaccgtgtggacccacccggagtcccaccaggactggacagcccccactggattatactggatatgtgggcatagagcttacgccaaattacctgaccactgggcaggtagttgtgttattggcactattaaaccatctttcttccaactgcccataaaaacaggtgaactcctgggcttccctgtctatgcttcccgcaaaaagagaagcatagctatagaaaattggaaagatgatgaatggccgccccctgagagaatcatacaatattatgggcctgttacttgggcacaagacggcttgtggggataccagacccccatttacgtgctcaaccgaatcatacggttacaagctgccttagaaataatcactgataaaatcagcagagccttgactattctggcctggcaagaaactcagatgagaaatgctatctctcaaaatagattggctctcgactacttgctagcagctgaaggaggggtctatgggaaatttaaccttactaattgctgtctacacgtagatgatcaaggacaagtagttgaagatatagttagagataggacaaaactggcacatgtgcccgtgcaagtgtggcatgaatttgatcctggggccatgtttggaaaatggttcccagcactaggaggatttaaaactcttataataggagttttaatagtaataggaacctgcttactgctcccttgtttgctacctgtacttcttcaaatgataaaaagcttcatcgctaccttagttcaccaaaatgcttcagcccaagtgtactatgtgaatcactattgatctgtcttgcaagaagacatggtagtgaaaatgaaagtgagaactcccactcatgagtgagattctcaaaagaggggaataagggaggagaccacccctcatactgtcttatgcccaatttctgcctccaaagaaagaagaagtaaaaactaaaaggcagaaatgaaatctgctcggcaccacacctgggcttggtagttaaagatcgacccctgacctaatctgttatgttatctatagattacagacattttatagaaaagcactgtgaaaatccctgtcctattctgttccattctaattatcagtgcatgcagcccccagtcatgtaccccctgcttgctcaatcgatcatgaccctctcaagcagactcccttagagttgtgagcccttaaaagggacaggaattgctcactcagagagctcagctcttgagacaggagtcttgctgatgctcccggctgaataaaccccttctttctttaacttggtgtctgaggggttttgtctgtggcttgtcctgctacaggtctaagtggaatctaagtggaggcatcactgagccagcctctccagcccagggatggggaggccccaggccctagtggacctggaccagtgatcacgtccttgaactacttcctacctgcctggggtggcccagagtacaggaagcccaaggagccaggagctttctcatctgggagcacaaaggatgtgaggaaaaaacggaacccagcccagcccagggaaacacagggactttcccctgccccgggcttcccatcttctttgcagagaccctgcctcttcttcaagggggatgattccagaccctctaagagcaacagggacacggagctcacatacataccccacatcaacatggagatcacacacctcaacacggagatcacacacatcaacatggaaatcacacacacatatcgaatggggattacacacatcaacgtggagctcatacatacaccccaacatggagatcacacccacaacacagagatcacacaagtcaacatggagatcacacacacacatcaacatggaaatcacacacacacatcaacatggaaatcacacacatcaacatggagatcacacacacacatcaacatggagattacacatacatcaacatggagatcacacacacatcaacatggagatcacacacacacatcaacatggagatcacattcgcacacatcaacatggagatcacacacacacatcaacatggagattcacacatatcaacatggagatcacattcacacacatcaacatggagatcacattcacacacatcaacatggagatcacaaacacacatcaacatggagatcaaattcacacatcaacatggagattacacacacatcaacatggagatcacattcacacacatctacatggagatcacattcacaacacaaacatggagatcacacacacatatatcaaatggggattacacacacatcaaccccaacatggagatcacacacacagcacagagatcacacatgtcaacatggagatcatacacacacatcaacatggagatcacacacacatcaacatggagatcacacacacatcaacatggagatcacacacactcgtcaacatggagatcacattcacacacatcaacatggagatcacacacacacatcaacatggagatcacacacacacgacatggagatcacacacacacatcaacatggagatcactcacacacatcaacatggagatcacacacacatcaacatggagatcacattcacacacatcaacatggagatcacacacacacaacatggagattacacacacacatcaacatggagatcacacacacatcaacatggagatcacacacacacaacatggagatcacacacacacatcaacatggagatcacacacacatcaacatggagatcacacatacatcaacatggagatcacacacacacgacatggagatcacacacacacatcaacatggagatcactcacacacatcaacatggagatcacacacacacatcaacatggagatcacattcacacacatcaacatggagatcacacacacacaacatggagattacacacacacatcaacatggagattacacacacaacatggagatcacattcacacatatcaacatggagatcacattcacacacataaacatggagatcacacacacacatcaacatggagatcacacacacatcaacatggagatcacacacacaacatggagatcacacacacacatcaacatggagattacacacacatcaacatggagatcacattcacacacatcaacatggagatcacaaacacacatcaacatggagatcacacacacacatcaacatggagatcacattcacacacatcaacatggagatcacaaacacacatcaacatggagatcacacacacacatcaacatggagattacacacacatcaacatggagatcacattcacacacatcaacatggagatcacacacacatcaacatggagatcacacacacatcaacatggagatcacacacacacatcaacatggagattacacacacatcaacatggagatcacattcacacacatcaacatggagatcacacacacacatcaacatggagatcacacacacacatcaacatggagattacacacacatcaacatggagatcacattcgcacacatcaacatggagatcacacacacacaacatggagatcacacacacacatcaacatggagattacacacacatcaacatggagatcacacactcatccacatggagatcacacacacacatcaacatggagatcacacacacatcaacatggagatcacattcacacacatcaacatggagatcacaaacacacatcaacatggagatcacacacacacatcaacatggagattacacacacatcaacatggagatcacattcacacacatcaacatggagatcacacacacacatcaacatggagatcacacacacacgtcaacatggagatcacattcacacacatcaacatggagatcacattcacacacacaaacatggagatcacacacacatatatcaaatggggattacacacacatcaaccccaacatggagatcacacacacaacacagagatcacacaagtcaacatggagatcatacacacacatcaacatggaaatcacaaacacacaacatggaaatcacacacacatatcaacatggagatcacacacacacatcaacatggagatcacacacacatcaacatggcgatcacaatcacacacatcaacatggagatcacacacaacacagaaatcacacacgtcaacatggagattacacacacatcaacatggagttcacacacacatcaacatggagatcacacacactcaacatggagctcacacatacacctcaacatggagatcacacaacacagagatgacacacacaacacagagatcacacaagtcaacatggagatctctctctctctacacacacacacacacacacacacacacacacacacacacactggcacaagcccagcattcatggcgcagccatctcttgtacttgctagcaaaacaccacctgtggaaagtcagctcaaacttccaacctcatccctcacaaagggggaatggagggtttggttttggctttcactggagtctgcaacaagaactggcatcatgctgcccatttcccgcctctccccacccagacccctgcctcagggacgcctgtcctcagcccagccctcagctgcagccaggccttcagcctccgtaacccccgctcagggtccccaccccctgcagccctgtccctccaggatgcatggccttgtcctgtgtgggggtggccgagagcactgccccagccctgggtaccttgggcaggaagctggcagaggccagggctgccattcaaacaggggcaggtggttttgccaggaggaagttgacagttcaacttcaaacatgggtgacgcaggccccacactgcctgctccccgtcccacccctccctgagcacgccaccccgccctctccctctctgagagcgagatacccggccagacaccctcacctgcggtgcccagctgcccaggctgaggcaagagaaggccagaaaccatgcccatggggtctctgcaaccgctggccaccttgtacctgctggggatgctgggtgagtacccctcccaggtgtcctgcgaacacccgggctcgctccagtgcaaggaaggagttcccagttttacccaaggctgactctgggatccacatgtcagccctctggagcgttgtggagatttggggccactgggatccctgcctgcccccactaagccgcagcttggccctctgtcctgcatgtcccacccgccaggagcacaaccttgcctctctcatgcgctgttgagaaccctgctttacccttccagtgcaagagagactgcaggggggacccgcatttgatggggcccagacaacttgattcctaggctgagttggattttagcagagcattcaggcctccctctgcgaggtcccccactgacagcccagcctttacttggtcgcctccagagacatggaaactcgccgtctccgaggcagctctgatgatgctctggacagacagtgggccgaggcagggtggagccgcctctgaggacgatgtctgagcaaatgtcaaggactcatagcggaagtcctaggccctctcatgcccagtcctgggtcagtcccagaggggcccagccaggccggctgggatgagtacacgctgggcctgctctcactccagccctgggttgtaagcccaaccctgtgtgacctcggataggtccccaagcctctccaggccttgatctccccatttatgaaatgtgggggttgggggtgtgaactggatgaggcctgacccacttcctgaggatttagaaggtctggcagggtgacagaggtgcgtgactctgggctggaaacaggaggatgcgagcagcttccaggctggcagggagagggtccggaggccttgcctgcctgagcagggagcctcagcagccttctagggaaaccgggtgtgcaggtgtgagtgtgggggaatgcatgtgtgtgtgtctgtgtgcatgtgtgtgagaactgtgtgtgggggaatgtgtgagtctatgtgtgtctgtgtgagtctgtgtgtacgtgtgtgagaactgtgttggggggtaatgtgtgactctgtgtgtgagtctgtgtgtgagtctctgggcatgtgtgtgagtactgtgtgtaggggaatgtgtgagtctgtgtgtgagtctgtgtgtgagtctctgggcatgtgtgtgagtactgtctgggggggaatgtgtgagtctgtgtgtgagtctctgggcatgtgtgtgagtactgtgtgggggggaatgtgtgagtctgtgtgtgagtctgtgtgtgagtctctgggcatgtgtgtgagtactgtgtgtggggggggaatgtgtgactctgtgtgtgagtctctgggcatgtgtgtgagtattttgtgggggggaaagtgtgagtctgtgcgtgtgagtctgtgtgtgagtctgtgttcatgtgtgtgagaactgtgtatgggggaatgtgtgagtctgtgtgtgagtctgtgtgtgagtctctgggcatgtgtgtgagtactgtgtggggggaatgtgtgagtctgtgtgtgagtctgtgtgtgagtctgtgtgtgggagtctgtgcgcatgtgtgtgagaactgtgtgtgtgtgctttcatgagcacaatgcggcctccaaaggcaggattatctccccaacacatgtgacaatggcacctgtgcacacatgttcacatggttcttctcacatgtaacaacaacaggtacaaaatgccttctgtcccaaagtaagaggcctgagacagacctctggctggcttcagggaccctgctaaggagggaccaggcccttaaaccccccacctgctcctcacctctggcctcagacttcattgagatagggcggctcccagctgagtatgggaacccgtgtcctgctctagggccctcccaactgaatcccatcatgggtgaggctcctgatggcaccaggcaagtcccctggcccaacacaggccacagtcaggaagacttggaggtagccccttcaccccagtatggggacctccttgaaccccaggaatactcagttcccagtgtctgagtgagccggtgggtaccaagcattccctttgttcagtgggtaacggtttacaaagcacttttaaagccattttctgctgcaagcttcacaacagccaatgaagtaggtgggcccattggccccattttacagtggggaaaagcgaggcccaggaaggccgagtgcaaacccagggtgcagcagctggtgcacagccgagctgaccccagcccagcattcctgccttcacagaggcccctcccttagctaaaagggaaggccaccaaaggcacttccgtgtgcaggaaaatggccctgggacacaacctccaccagcagagatacagccaccagagctggggacaccaagggccagggttgctggagtcaaacagtgcaggcaccctttgagccattccaaggagagaggcagcaatgccagagccctggggacaccacagcctcctggctggcatcggggcttggggacctttggatcctgaggcatgaagatgcccccagcaggcctgggtgctagtgacaggccagggcttcccacagttgctcccctctcagccccagcctgcctggacagctgctggcagtgaggcggggaagcggctctaacacaaactgcacgtccctgactggcctagccgatgcgactctgaaagccagcactgggacgtgggaaagtcatggactctgcagtttgaattccagtctgccacttaccaactgggcgaccttggactagttctgtaagcctcagttttctcatccgtaaactgggtgcaacagtacctcatagaggcttataaggattcaatgaatcaatgcacacagagcttaagtacaatcctggcatatagtatagtaagtactcaataaacaataataacagcaacaactatgattattattattaccagggtcagcaagaaacaatgcgaggtaggagccaatggggaggagggtcttgccagaggcctgcacagccgtcaatctctctacccctcaatcctcttgcacaggctatttattcactaattcattcatttttcatctacaaatattattgagcatctcctgtgcatccagctctgtgcaaaacactggggatacagggaccaaggcaggagtccctgccctcaaggatttgatcagctaacaggagagctgttagagaacacaagtaaacaagcaccaaagaccgggcatggtggctcatgcctgtaatcccagcacttcgggaggccaagacgggtggatcacctgaggtcagaagttcaagaccagcctggccaacatggtgaaacctcatctctactaaaaatacaaaaattagccgggagtggtggcacgcacctgtaatgccagctactcaggaggctgaggcaggagaaccgcttgaacctgggaggcggaggttgcggtgagccgagatcgcgccattgcactccagcctgggcaacagagcaagactccatctcaaaaaaaaaaaaaaaaaaaaggcacccgaaatatttacacattttgaaatactgagtgcaatgaggagaacagcaagttcctcaggcacaatacagtctgcactaggataggaggacatccctttggctgggcaggcagggagggcctctctaaggaggcgaggtctcatctgagaatccgagacctgaatgagggacggagccagccaggcagagaaaaaagggaagagggtcccaggcagagcggatggcagggcaaaggcaagggggaaggacaggcctgagagggtggggctcctactgggagcagggcagcaggcccactgtcccaggctgggcgaggccctggttggatcatcctcctcgaagacccagagctccccacctccagcagggaatgaagggacctgggggcccaggaagggacctggccttcctgggtcactctgtcaccatcagttcagaggggttttccctgcagtgaccaggcggcgaattcagctcaacctgacaaaattgattgaacatctattctgtgtgtgtgtgtgaaattgaacatctactgtgtgtgtgtgtgtccatgtgctgtgtttgcgtatgaacacatatgtgcacagccctgtggaggggagctttggagcaatgcagacctagtttgatttctgcttggccacccacaagtgaacatctctcagggtctcatctataaaaataggggttccttgctgtgtccacctcaaaagatcactgcaataattacatcgggtttattcagagactaggtatggattgaacacctactatgtgccagatgctgttctaagtgctgaggacacagcagtgaacaaaacggtggaaaatccctgccctggtggatctggcataatagtcagggtgtgatggggagacaaacaagacacaagctaagctagtgattatgcgaatactagacggtcatgagaggtgtggagacagacagatagaggagggctgggaccggaagccttggtggggtgtgaggtacctggggaggattcagcggctctcatgagaagatgatatttgagtcaatatttgaaagaagggagtgagcccaccgtgtgggacctgaggagaagcgttgcaggcagaggggcctccagctgcaggtcagtgggggatcgatgaggcagccacgagggccacggatgcccccgtgaggacttgggctttttctcccatgcactggacagccacacaggagcagagaggtgagcataactctcacctctccaggtcctcaagtgcatcttgaaccccaggccagatgcatttggaactccagaagctttcagattgtagaaggaaatactgtgtacatactgtgtgctcttaacacacgcagtgaggcctggggcagcaccctgcgttcaacacattcccatttctgcagtgaaacatgaatttgcacacttagagggatgtaaagacagcaaacaccctcccaccaaatgcattagaacaagacttttagattctggattttgaattgcagataagagactgtgggcctggatcacttcttctatagctaccactatcaccgtgaccagcatcatcatcaccatcatgtacctttcaaggcctgactttcatggtgtccagagggaccctaatactcctctccgggggacagatacggtaggaatagcgtaggaaatgggtggtggcagtctgcagagacgacaagggtcccatgcactgccacagattcccacagagacatctggaatgaacccctccctccttctggccccagtttcccccatctgtactccagagcaagcaggctctggggcctccctggggtagactcccacaggccctgtcaggtgatggagacagggtttctctcctgactctaccaccagctagctgtgtgaccttgggcaagtcactcagcctctctgaacctcggtttcctgatctgaaaatggaggtgagcatggtcccttcttcacagggctgtgggtagaaagaacaagtctagtgcatgagagagctttggggtaggtaaagctctaaacaaaaccaaagccttgtctctgtgacagcagagacaccgtatatggtgcgaaactcatgcaacagcagactactagtgggagagagaaataacaattcaaacagcaaaggtcattctgcccacgacccactgtccgctcactgggacaggccctgggtgagccagattagagacctgaatctgctgttccttcctgcgggctccattcccaggcctctcctggtgtaagcccagtgtgactctatatgtgttttgcacataaaagccgcctaatcataaaccaatcatttcatctgtgctcaacccaagaaagggaattttggtcctttacgaccttcgtttccctaaagccctgatacagctccaactctacagatgactgtcgtttcagaaaagaggggcttctgacctttcaagtcagccaacatataggattcgaggaaggaaaatccagaatcctacccaaaacacaggcagatctattgtatttcatggacatcgatggctgtagctatggggaggtctgtggactgttagaaggaatcactgtttacgggaaaaagggaacacctgacttgcaaactacactaaatcagtcataggtgttttatctgtggaacaaagtggtgatagcaccccgtcttctgaggctctagaaagaaatggggtcttctgtgaaaaatccttgtcctggggtctgcacagggtaaggatgctgtcattttattatcatcggtcattatcacaggcctgtgaatccgggtgcctgccaaaccctaagcaggccacacccttgcctccatttcccagggtgcaggggcggcaggacctaggagtgagccagaggcaggggccaggagtgcaggttacaaagccagccgccctgggctgaattctggccctgccgagttctcactcccatgcaccacgacagcctcctcctctgtaaactgggacccatcatgcaagtcaacaaatattcatctgtaattccaaagcccaaaaaacatttgtaagtttctggcaaatgcatttggtgcaaatcctgacctgaacgaatgggaggctatttataatcttgacacatcccccttagtgtgaaactgcatatgtttcgctgcagacactgccatgtgtttgaagccaggtctctccacacacatggcgaccgtaaggtgccagcgaggcgtggacgaaacagaagtcaagggccaccgccactgcctggggcctccagagatgcagccagtggagccaggatgtcctatttttcaaaaacgcctggatcttgaaatgtgtttaaaaagcaaaacaaaattaaaagaaacctttttttttttaatttggctcaaaaaatttttaaacactgtgcagagcaaataaaactcatgggatgccgtaatgagagccaaagtgagaattatcctgaatactgtgggggcaagaggaatgagagggacagggaggaggatatcctcaccaagacctcagggcaggcagggccaaaaatattgtctctgccaatgcaaagacaagagacgggagctccaggggtgtgataagccgtctgacttctttagccctgaattttgcctgggtttgcccagtccaggccctaaattgtgggagctgcaaggggaggtgggcaggagaaggaaggggtcttcagagaattctcatccgtagccccagttgctcggggtgaggggtggctggcggataaggaaacggcatagcacttccaggcctcgggggcctcctggtctggagtctcggggattctgtccacagttttggcaatagcaggagcctcaggcttcatgaggctaggaaggatggtgcaggagtaagctgtagagcccccagagggggtcagggagaccctgaacacagaccgcagggctcaggggatggagggtgctggtctgacccctgtgagaagcaactaaaaaatagaaataggttatggaccctcaaaacctcaatgttcattctgctccctagattataagtgaaaaacaaacaagcaaataaaaaaaaaacctgaacatattcatggacaggctgaaagaatatacacaacacactcaggaagcttccaagtcagctgtacatctcaccgccctccctccaccagctgctgaaatccatcaggagctaaagattatagatcacaggtgtttgaaccagggggaaggtgagggactgtggaatctaacttccttactttacagattaagaaactgagcccagagaggctgagtggcttgccaaggtcacacagcacttcatagctaagtcttctcactgcatgttttactgactgaacggaatcacaagttcattccctttgatcaggattgcacaccagggtggtgcaggaatcaatgaaacctggggcaggccatcatgggcttcctgctggagggaagggaggcagtttgggcctcactggcaaaagctcaagactcagtgccaggcggctaggtttgagttcgggccccagcactcattagctgcttggacaagtcatttaacccctctcaagtttcctggtctgtaaaatcgggaccaaaataatagcacctacttcatagcactgtggtgaggactgggagagttaatgtgtaaaaggcccggaagagtgaaaacttttaaagtgttggtgatggtgacagtgatggtgatgatgatggtgacggtgatggtgatgatgatggtgacggtgatagagatggaaggagaggagaggaactgctgctcatctggggaccagacaggcacccagaggaaggttccagggcgacactctagaatgcataaatatcttttttattaaagcatttgttagagcattttttaaagaatttcacttggttctccagcttgttcagtctttcctgagaatgttctctgagcaagacctaggctgttctgtgtggaatctgccaagattgtcaggcccagtccctgtccccaggccttcagggtctggctgcacgagcgcttccaggtcagacccaggtggtgttcatctctgcatctccaaagctggcactctggaggagcaattgggtggctgggagcagagagcatgggttttggagtcagacacatggttcgactccacctgccagccatattacccgggacagggcatttgccctctcctgacatttataccaggaacgtactcaactctcgtgatggggctgtgtggaagattaacaacaatatctgtgatgaaccataccgtgggctaagtatgcagcaggtgctcaacagttggcagctgctgttaataatgaacgttgagtatgatgactcgggaaatagagatgctccttgacctacagtggggttgcctctcaataaacccattgtaagctgaaaacatcctaagtcaaaaattcatttaataccccgacaaacccattgcaaacttgaaaagtcgtaagttgaaccaccctaagtctggaccatctgcgcgaagtttccaagttaggagaaggaagtggtgagccccatggcttggctctctgtgctcctggacagggaatcttcccactgggaaagctccccgagggtctgctgccctgggcacagcttcccccagcccaccctactccacagatcagtagcaccagctctgagcactaggccaggaagcactgggctaagccctttacaccgattagctcatctcatcttcacaacggctctagaaagctagtcccatgctcatcctcatgctatgagtgaggaaatcgtggcttggaggcacagagacaggacagacgtgctccccagactctataggcaggaagtggcagagctgatactcagaactggagcagtagaaagtcatcggtgactgaaaacccatcctgagctgtgtagcactgaagggcagatatttggactcccactccccacaggtggcacttcaagctgccttagccctcgagacttagccagagggatgactccttcaaggcagactgccctgctcctgatgggcagctgggatgagagggaggtgggaatgagaggtcaggagtgagaggtgagggcaggacaaaagggcctccttcctgatttctctcgctagctggacatctccctgatggcctcctgaggctgccctgcccactgcaatagatcagatgattggctgggcgcggtggctcatgcttgtaatcccagcactttgggaggctgaggggggtggatcacctgaggtcaggagttcaagaccagcctggccaacatggtgaaacctcatctctactaaaagtacaaaaattagtcgggcgtggtggtgcatgtctgtagtcccagctactcgggaggttgaggcaggagaattgcttgaaccagggaggcagtggttatagtgagccaagatcatgccactgcattccagactgggcaagagaggaagactctgtttcaaaaaaaaaagaaaaaattgatcagacgattgacaactagaccatccttttccacccaggccagagctctgtacacttccctgttggtgagaataaccttccccttccaaggcccgtgactcctagagcaacagccacgaaaggatcttgcagattcaggggaaacttatcacatggccatgatgacttcattacaaagagtcttgattcggggacaaggatgaactgtggagcacatcaccccttaaaattcaaccgccagcttgagaacactcaagcaagaacagctttttcaaagcacacctattttttaagagacggggtcttgctatgttgtccaggctggactcgaactccttggctcaagtgatcttcctgcctcagcctgctgagtagctgggactacggccttgagccattggcccagctaaacacacctattttttaacaccaagcaaaaatttctcacaaacaatttgaacacagcctttcactattaatatataaagcagtctccttgacccagtgccgtaaatccaagagtaaacccaagtcggacagatggaaatcaaaataccgtacttgcacctaactgatcacgctaggtggtgaatacagaggttaagctttgaagccacgtgagcttgctacttcacctcgtccccacccaaattaaacctcttccccagccacagatgagctgctctgctgagagccccgcaatgggcaactttgcccccactccacagtgagaccaggctacaaggaggcagagcttccacgtggtccacatgggggaaggctggttccctgcagacccgtgttccctgggcctgtcgccttggctcccttgtcaactagctggctacactactgtgggcaggtcacgccatctctttgagcttggtcttctccagaaatgggcagctgttaaagataagtggctgtcattcttatcctgccttgggaatgctagaaagtagcaaatgttctcttggttccctttcttgctcatttctttctttccttttttttttcttgtaaagacagaatctcactctctcacccagtctggagtatagtggtgcaatctcagctcactgcagcctccacctcccaggttcaagcaattctcctgcctcagcctccagaggagctgggattacaggcatgcatcaccacgccgagctaatttttgttatttttagtagagacagggtttcaccatattggctaggccagtctcaaactcctgacctcaagtgatccccccacctcggcctcccaaagtgcttggattataggcatgggccaccgtgcccggcccctttctggatcatttctccattctctagaactaaggagctcaaacttttttgagtggaaacatgcgggacatttttacattgcaacccccaacacacaaaaagtgatcagtgcattctgacattctcttttttatttatttaaaacaaaacaaacaaaaaaaaacagggtcttactctgtcacccagctagagtgctgtggcataatcatagctcactgcagggtccaacttctgggctcaagcggtcctcccaccttggcccccttaagtgctgggattgcagtcataagccaccgtgcacggcctctctattctactccatttcatttttaaaaaaattgatggcctgagtgctgtggctcacacatgtgaatcccagcacttcaggaagctgatgcacgaggatcacttgagcccaggagttcaaaaccagcctggacaacatagtgagaccccatctctacatacacacacacatacatgcatacatacatacatacacacatacatagtaaaaaaattagccaggcatagtggctcatgcctgtaatcctagctactcaagaggctgaggtgggaggattctttgagccagggaggttgaggctgcagtgagctgtgatcatgccactgcactccagcctgggtgaggagagtgatatcctgtctcaaaaagtaataataataataattgatggttacattattacaaaggttagcatgagggaatttgaggtagggagtaatggaactggtgtataccttgattgtgatggtggtcacacacatctatatatgtgacattcacggaatcatgcagtaaagaaaaatcaatttcacgtctgttcattttaaaagtaacgttttttaagaagaaaaaaaatcgatagttgcagcccactagatagaattcatatcactcaggggttccaacctggagtatgaaaattcctgtccctaaaacccatgatagtggatagggggaggcagaaagggccattgctcgggctgtgggtgggtgagctggggagaagggagagagtgggaggtttcacttcctgaccctcctctcttctttctgcagtcgcttcctgcctcggacggctcagctggtatgacccaggtaaggaagagccacatggagaaaggcctggggcagggggagagtggggctgtggtttcatcaggccatcggggacctctcgatgaagccatcacttctgccagagtgaaccccaccctatagagagagtgaaccccagcatacacacaggcacatagatgcagacactgcacattaagatgctcacatgcaggtgggtgccctcgacagccgtaaatcacccacaaatgccagatctcatgataattattatgacccgctcaccatgcacagaagacatcccagctcataaatgtaccttgcaaagtcttatttcccacccaatcctgacagatgctccatggtcaaagatgtttagagcggagtctgcagagagaggccgcagactgatggtaaagtgtgtggaacgtccagccttagacgttggagtttagtcgtagaggctgtttcccaaatagggttccatggagcatgttggacaaagggcaggcaacaccgcatctcccactgaagactgacagtgtacaccggcccagtaaaggatctgagaaatcctgcagcgaagaaatacatgacactttttaacccagcactttgcacccttatttcaccacagacctctccttcacattgactgggataacatcgtgctgaatgtacctggggagaattgtggctgaagggtcttgagatgccactgtgcagccagagggggtgacaggggccttggcaggccctaagcacgatctgggccctgcactgacatcctagctccaagctttggcacactgcttgacctcttgagcccgtttccccagctgtgaaatgggaagaaaatctcttgcctggtgaagcggcaacgagccagctcgctgggcggacagcctgcaaatcaccccaccaatgttaggtgttgagacgcgctcgttctccctctaaccagccaagtgcccttggcgagccctggccctgagctttggccccagagtgtcctcgtcggaagaaaagggtagcctgacccgtggttcccgaccagggaaaacctccccctcagaatcacctgggagagcctttcacatttctaaaaatttcttgaatcagtgacacattcatccagctcaaaaatttaaaaaaatataaaaagatctgtagtgaagagtctcacttccatctctattcccgtctatcccagtcgcccccgacgtcgaccccaacaccccaggtaatgactacgtttatttgtttcttgcatattcttcaaggagctctttatgcaaatacacacacacacaccccacacagaccccacacacacaccacacatacacacaaccacaccacacacacacaccaccacaaacaccacacacacaccgcacaccacatacacaccccacacacaccaccacaccacacacacacaccaccacacacacaccccacaccacacacacacaccacacacaccacacacacaccacacacaccacatgcacactacacaccacacaccacacacataccacacacacaccacacacaccacacacaccccacacacaccacacacaccacacacacacactacacacaccacatacacaccacacacaccacatgcacactatacacaccacacacacaccacatacaccacacacacaccacacacaccacgcaccaccacacacacacaccacctgcacactacacacaccacacacacatacaacacacacaacacacacaccaccacacacacatcacacacaccacacacactacaccacacagacacacacaccacacacacacatatatttttcccctcatggagtttgataaaatcacacaagacacatgtctacccatgacccagcaattccattcctaggtatttacccaagagaaatggaaacgcccacacaaagacctgtatgtgaatgttcacagcagccttattcatagtaggcaaaacccggaaacaaatcaaatggtgaatggaaaacaaattggcatgtccgtgtagtgaactatcattcggcaataaaaaggaatgaactacttatgcgtccagtgacatagctaaatctcaaatacatgctaagcgaaaaaagccaaatcaaacaaaataaaatccatgctgtatgagtccatgtctatgacattgtagcacaggcaaaactcatcttttgtgacaaaaagcagatcagtgtttgcctgaggctagattcagggaaattaactataaaggggccttagggaactttctagatgatggaaatgttccatatctagattatggtggtggttacatgcatgtatacatttgtcaaattccatcgaacagtatccttaaaatgagtgcatttcactgtatgtaaaattacctcaataaaattttttttttttgagatggaatcttgatctgttacccaggctggagtgcagtggcgcgatcttggctcactgcaacctctgcctcccgggttcaagcaattctcctgcctcagcctcccgagtagctaggattacaggcgcctgccaccacacccagctaatttttggtattttttctttttttagtagagatggggtttcaccatgttggccaggctggtctcaaactcctgacctcaggtgatctgcccgcctcagcttcccaaagtgctgggattacaggcatgagccaccacacctggcctaaaattaatttttaaagatctcttaaaaagcagacaccagccccaatctcagaccccttgagacagaatttccaggacaggggccatcctgctggacagtgggtgccgagaacaccttgcccatttatctgagctcccttctgactctgaaatctggagccccaccctcctgggtctagcttcggggctgcctgggtcagggtcctctgggaagcccctgcagtgccccagaagggacgaagctcacaaggggcaaggcaggcagcccacggggcaggagggagctcaactgggcgtcctagggagagggcagtgaggggtgccagtggggaacccctcccagcctgacccccaccacacctttctgacccccagatttccaggcaaggctcacccgttccaactcgaagtgccagggccagctggaggtctacctcaaggacggatggcacatggtttgcagccagagctggggccggagctccaagcagtgggaggaccccagtcaagcgtcaaaagtctgccagcggctgaactgtggggtgcccttaagccttggccccttccttgtcacctacacacctcagagctcaatcatctgctacggacaactgggctccttctccaactgcagccacagcagaaatgacatgtgtcactctctgggcctgacctgcttaggtgggtaactagccagccacacgggcaccctgggcctgggcgccagccccgaggagactgcccgaggcctgtgatctagggtctgagcaggctggtggaaggggtggggggaccccagtttataaccactccccaagacacatacccaggagggggactggaaggggccagcacccatctgtaggatggcaatggaggacctagttctgccaatcactgacttcatcgtcgcctctgaacctccattctcccatctgtgaagtggggtggtacttcccgcctcgcaggaggcttagagacaacgtgtgggtcaagtggacctggtgtgccaagcggcactcatgccaggagctcctggtcctctcaaggctgctggctgcccccggccctccccacaccacccattcctccctcaccagagtgtctcattgcagaaccccagaagacaacacctccaacgacaaggcccccgcccaccacaactccagagcccacaggtaagaggattctgaaccccccacagggagtcagagctagcaaataaaaacccaggatgcccagttacattggaatttctgacaaaggtggaaatgtttagtattggtgtgttctacgcaatatttgggaccccatcacctcccaaggctaagcgttagtcagtagttgtccacaagttggggccaaacagcaaggagtgcccaggaagccctcggcgctcagggtggctccccctcctgctctctcctctcctagctcctcccaggctgcagctggtggcacagtctggcggccagcactgtgccggcgtggtggagttctacagcggcagcctggggggtaccatcagctatgaggcccaggacaagacccaggacctggagaacttcctctgcaacaacctccagtgtggctccttcttgaagcatctgccagagactgaggcaggcagagcccaagacccaggggagccacgggaacaccagcccttgccaatccaatggaagatccagaactcaagctgtacctccctggagcattgcttcaggaaaatcaagccccagaaaagtggccgagttcttgccctcctttgctcaggtaagtgagacctggccaagccccatgacaccttctgctgccctaggtggggtcacagagcatcccagaaggtcagggaacatgtgtgcagcacagggcactatggagaatacaagggaagtggaggcctggtcttggcctctaagaggtaacaagggttggggtggggaggatgcatccacactcaatgccttggtaatctctgcaaagctacacaccccaagcccaaaggaaccgctggctcagctgccacatggggaaggggagtcggcaggacttccaggaggaggcaaggtctcttcgtgggtctgggggagaaaaaaaaatgtctaggtaggaattatggcttgtgccaaagatggggcacttggaggggtgggccttgcaggaagggcatttgtaaatcccaagagttggcattgcagcaatggtgagaggtgggaggtccctgaggcccgggagtcagaggacagcaggcaaagagagaaacagagacagcccccaagagggctctgcagagggtcctgcgccgtgtgctgacaaggtaaagagaatgatgccgctgtgtggttgaggggaggaggcaggtgagactgagtctgtgagaccccggtgggccaaaccaggatcaatgaagaaaactaaagggaagaaaccacagctgaaacgaagggagaaacggagaaacttcgcagtacgcctccctccggaagtagtgggttcccatggccggaagtgttctcagtgaagtccacactgtacttaccgagagcttacttcatgcagggaccattcctggacattctaaatttttcacatggccttaggaggtgggtgttattattatcatccctttttacagtgaggaggctgaggtttagagaggctaaacctctcgcccagtactatggagcacttgctgggatttctgttcaagcagggtcactctagagcctgttatctgagccttggtgactcagtgtggtcctggaacagcagcagctggttagaaatgcaggtgctcaggccccagcctggaccttccgaatcagaatccacttttcagcaaggtcctcagtgatttatgtgcacaaatctaggaatcactgctcggggccacctggctgtcctgcaagcacgctgggtacctggtgtaaattagtaaaatgctgcccctcccccagcagaagcagctcccaaggggctcttggcctgatgaggagagcaggggctggctcgcagtcccaccggcccctcagctactatccttcctgcaggctacaacctgtaaaaccatacccagggttcttgcctttgaagcttcttctaaatagatttactagggctttgaaggtggatgttgctatgggaactgggcaaagctggtgagatcacaactctctctaaactgagcatgtgtgggcctcagctacagactcccaggccccaccctggcaggcagaaataaatgtgtgcctctgttgagccaggccccacttgctgaaagcctggccctcagcgttcagtgcctgggcagcacgatctgtcccaactgtgaactgccaggactcagggcagtaggtctttccaacccccaggcctcagtttccctcttctgaaaagtcctccttcttcccaaagtgcagcgggataatgacaagatgtttccaagtgagaagtggacctaggtggagcctatttcccacgctcttctcttatctccacctggcatggagccgactccctgctggtggctgccaacagcctccctgcagaaagcgcctatgctgctgtgtctcaggatggtgctggcagctgcccagaggcaccagagacagtgctgggtcaggggaagagccacagagggtgccagaaggagggaagggcagaaaagaaggctgcctctcctggtggtcccacacagtgccttcccctagccagggccccgatttgccagtggcatggggccccaggaagcagcacacaggctcaggttcacacttgcaccctcctttcccattgcttcccctctcaggtttccagcccaaggtgcagagccgtctggtggggggcagcagcatctgtgaaggcaccgtggaggtgcgccagggggctcagtgggcagccctgtgtgacagctcttcagccaggagctcgctgcggtgggaggaggtgtgccgggagcagcagtgtggcagcgtcaactcctatcgagtgctggacgctggtgacccaacatcccgggggctcttctgtccccatcagaagctgtcccagtgccacgaactttgggagagaaattcctactgcaagaaggtgtttgtcacatgtgagttggccacagcccacagtgggtggaagcagttactactttacctctaggacccggtcagggtgcatgtctctaaagggaagccctggggagctagacagagagtcccagagacccagaaaggatggagacagggaaattggaaggctagggagcaagagtactcagacagcaatgggcatgaacaataataggagacagggcaagcaacaggcagatcactgtcaggcagctgatgagtcatagaacttctccttctaccttctacttttgcaaggtggaagccagaaaatcaaatggtagatagcacatatttgctacatgaatggtaactaaacgggaagtaaaagagagaaatggatggatggatggatggatggattggtgggtgggtgggtgggtgggtggatggatggatggatggatggatggatggatggatggatcggtgggtgggtggatggatgatggatggatggatggatggatggatggatggatggatggatggatcagtgggtgggtggatggatggatggttggatggatggatggatgagtggactgatggatgggtgagtggatggatggatggatggatggacagaagggcggatggacagatggatggatggatcaagaatagaggccccgctggctattggtatgcaatcatatattggtttctgatggtatatgatggcaaggtgatcactaataactggcaaatggtagaaaattaaatagcttataacaagtgtggcaaatgatagctagatgatcaactgttagatgacaaatggtgggtgggtgagtgggtagcagggaatcctagagatagatggagggatagccaaatagatcattgccttccgtgcatgctggtgaatttctcagatgagcaaggatgacggcggaagccaggcagccagcagcttccctcccacagtttttctccccccaggactgggactgacctaactcttcctccttccccaggccaggatccaaaccccgcaggcctggccgcaggcacggtggcaagcatcatcctggccctggtgctcctggtggtgctgctggtcgtgtgcggcccccttgcctacaagaagctagtgaagaaatgtaggtgtcacggccctgagtggctccgttcccacgtgcagagactggaggggctgcactagagtcctccggaggaggggtcatgcctccagagagctgggcacgcaggacacctctgcttcaccacccagccttcccctctcctgcccattagccattttctgccccaagtaacagaaacacaaagtaaacaaaattgcaattgctgatttgtccatctctggagcccagctgagacccactccaggtcagagagaaggtgtggccattcccaaggctgtggcaggcccagagggtgttcagtacagcctctgatggtaggagacacacatcccaggcctagccccaccacttaccactgcccaagatgctgattgggacacttcctctctctgaccctcagtttcttcatctaccaaatgggcaatcgcacaccttgtgaacgcggggggttgttctccttgccttgcagaacttaagaggaaattattcagacaaagaagtgagaggggagagattgggaacagagggcccagcaggacagtgttaggtcctagtgcctgcccaggtctagttctaaacgcctgtggtcttttctcctggggagccgggcacctctacaccatcctcggcctgctccttcggcacaggatggctgcaattcccttcccgagcaagttaggaaactgaggcctacgagagactccagggggcagagcccagcagccctgccctcagctgcacctgccgccaccatctccctcccaggcccagccccatccccacccctgcctgcccccacccatacctgcccccaccactctgatgtgcctttcttgtctcttgcccagtccgccagaagaagcagcgccagtggattggcccaacgggaatgaaccaaaacagtaagtgtccccggaggcaggagcctccctcaggccctggacagaggccatggaggcagagtcgaggctctctgctgaccacagacggagcctgtggctgcttgaagcctctgatctccacggtgcttgtctgacatgtgactgtggccaacagacgtccccacaccagggagggcggaggcttcttgatgggctgatggctgagcggtttctaccgctgaagcctcagagtgtagaaacatctcatggtatcctttctgtccctcgccccatctctagggtccccagcagccctcacctcctccagcagtattccccaacacagtagccagtggatgttaacaggcagcactcacaagaggcctccctgatcaacagatctggaaaaccatgagttaggcacagctaagcatagctatttcaggacttatcagaaccttaatgggcatttttgtattgcccatctccaaagcaaggatccattaagaagggctgtccaagttattttcatcatggaacaacttacagggccagtattaggacattttggtgcagccagcccaacaccatccctggtcttcatcctccctccttccagccacagtcagagacaccctaatggatggagacatggcgctcttcctgcctcccattctgccttcttcccccttcccaagtccacagccaaatccttcaagagggctctgttgaatttgttattaagaaaggctctggaaactttcttaataacatacaggccctcagctcctggagggcagggagcagatctgtcccgtcacctgtgctccagtctcctgcttggtaaatgcttgatgaccgactcacggaaggccccaccctgccagtcagattgctgggttacccaaacctgctctaaaagtctgctgctgatcctgctagcatgaacctcccaggaagaaaggagagggggatgcattgaaggggctgtgggtgcctttgtcctctccccgctaacatcatgggaataggagattaaagttctgggcacctgctggggaaggagacggaggacacagccacaagtctgacactgtctcctaccatctgcccagtgtctttccatcgcaaccacacggcaaccgtccgatcccatgctgagaaccccacagcctcccacgtggataacgaatacagccaacctcccaggaactcccacctgtcagcttatccaggtaagcaccagcgggtgctcccaggcacgcaggcagggctgcagcaggccgccaaggcaggtcacgtgatgcccttgaaggtcgggtgatggcccaaagacagaatggagaccccagggtgcaaggggatcaaacatcgcaggatgcagcaggggtacggaagggatatagcctttgcaggccccagctgtcccgtttgagaccacaaactactcatctggagagaagaatgcactcagaaagccatcaaccccagccttgctgtgacctaatgggcctgtttcccttcagggccttgaggggcctggatggtaaatgaggccccttggattcctccgggctagaagagagccattaagtgggctgcaggagcgctgtactaaaggctcccagactggcactaccaattaagccccagggagcctggagttcagcatcattagtgaacgtttggctgatttgatcaaaacagataaggggttgtcaccggctcataaagcccctgtgatcttccaacccactgtgggcagcggctctaggatccaaggggctctgtggagtcaaaaaccctctgcaaacagcgtcctttctttccccagctctggaaggggctctgcatcgctcctccatgcagcctgacaactcctccgacagtgactatgatctgcatggggctcagaggctgtaaaggtgagcccgtctccagcctgaccccagcaccccagggtcccccacagtcctgggggtgagcagcagccactcaatgcctcccctcagtcccaccctgcccatacatgaataggggacccccagcatcttgattcttgtaaagggaaagacaaacgtcagctgtttaggttatacgagctgatttgacaattagagcagagcaatcatgaactttaacaatcaactcttaaactccattcggaaggccacacaggcagagtccctaagagctccgtgatggagtcacacccctctagagctgccaacctgccagcgggcagaaggagcccctggctcaggacagcagccgtccccaagatggagcccagagcatgggtcctgtccccgacctccctgcatcacctctgcggtgtcttcctctaacacg

SEQ ID NO: 183 - DNA, 3127 bp, Homo sapiens
  source 1..3127: mol_type = genomic DNA
gagatacccggccagacaccctcacctgcggtgcccagctgcccaggctgaggcaagagaaggccagaaaccatgcccatggggtctctgcaaccgctggccaccttgtacctgctggggatgctggtcgcttcctgcctcggacggctcagctggtatgacccagatttccaggcaaggctcacccgttccaactcgaagtgccagggccagctggaggtctacctcaaggacggatggcacatggtttgcagccagagctggggccggagctccaagcagtgggaggaccccagtcaagcgtcaaaagtctgccagcggctgaactgtggggtgcccttaagccttggccccttccttgtcacctacacacctcagagctcaatcatctgctacggacaactgggctccttctccaactgcagccacagcagaaatgacatgtgtcactctctgggcctgacctgcttagaaccccagaagacaacacctccaacgacaaggcccccgcccaccacaactccagagcccacagctcctcccaggctgcagctggtggcacagtctggcggccagcactgtgccggcgtggtggagttctacagcggcagcctggggggtaccatcagctatgaggcccaggacaagacccaggacctggagaacttcctctgcaacaacctccagtgtggctccttcttgaagcatctgccagagactgaggcaggcagagcccaagacccaggggagccacgggaacaccagcccttgccaatccaatggaagatccagaactcaagctgtacctccctggagcattgcttcaggaaaatcaagccccagaaaagtggccgagttcttgccctcctttgctcaggtttccagcccaaggtgcagagccgtctggtggggggcagcagcatctgtgaaggcaccgtggaggtgcgccagggggctcagtgggcagccctgtgtgacagctcttcagccaggagctcgctgcggtgggaggaggtgtgccgggagcagcagtgtggcagcgtcaactcctatcgagtgctggacgctggtgacccaacatcccgggggctcttctgtccccatcagaagctgtcccagtgccacgaactttgggagagaaattcctactgcaagaaggtgtttgtcacatgccaggatccaaaccccgcaggcctggccgcaggcacggtggcaagcatcatcctggccctggtgctcctggtggtgctgctggtcgtgtgcggcccccttgcctacaagaagctagtgaagaaattccgccagaagaagcagcgccagtggattggcccaacgggaatgaaccaaaacatgtctttccatcgcaaccacacggcaaccgtccgatcccatgctgagaaccccacagcctcccacgtggataacgaatacagccaacctcccaggaactcccacctgtcagcttatccagctctggaaggggctctgcatcgctcctccatgcagcctgacaactcctccgacagtgactatgatctgcatggggctcagaggctgtaaagaactgggatccatgagcaaaaagccgagagccagacctgtttgtcctgagaaaactgtccgctcttcacttgaaatcatgtccctatttctaccccggccagaacatggacagaggccagaagccttccggacaggcgctgctgccccgagtggcaggccagctcacactctgctgcacaacagctcggccgcccctccacttgtggaagctgtggtgggcagagccccaaaacaagcagccttccaactagagactcgggggtgtctgaagggggccccctttccctgcccgctggggagcggcgtctcagtgaaatcggctttctcctcagactctgtccctggtaaggagtgacaaggaagctcacagctgggcgagtgcattttgaatagttttttgtaagtagtgcttttcctccttcctgacaaatcgagcgctttggcctcttctgtgcagcatccacccctgcggatccctctggggaggacaggaaggggactcccggagacctctgcagccgtggtggtcagaggctgctcacctgagcacaaagacagctctgcacattcaccgcagctgccagccaggggtctgggtgggcaccaccctgacccacagcgtcaccccactccctctgtcttatgactcccctccccaaccccctcatctaaagacaccttcctttccactggctgtcaagcccacagggcaccagtgccacccagggcccggcacaaaggggcgcctagtaaaccttaaccaacttggttttttgcttcacccagcaattaaaagtcccaagctgaggtagtttcagtccatcacagttcatcttctaacccaagagtcagagatggggctggtcatgttcctttggtttgaataactcccttgacgaaaacagactcctctagtacttggagatcttggacgtacacctaatcccatggggcctcggcttccttaactgcaagtgagaagaggaggtctacccaggagcctcgggtctgatcaagggagaggccaggcgcagctcactgcggcggctccctaagaaggtgaagcaacatgggaacacatcctaagacaggtcctttctccacgccatttgatgctgtatctcctgggagcacaggcatcaatggtccaagccgcataataagtctggaagagcaaaagggagttactaggatatggggtgggctgctcccagaatctgctcagctttctgcccccaccaacaccctccaaccaggccttgccttctgagagcccccgtggccaagcccaggtcacagatcttcccccgaccatgctgggaatccagaaacagggaccccatttgtcttcccatatctggtggaggtgagggggctcctcaaaagggaactgagaggctgctcttagggagggcaaaggttcgggggcagccagtgtctcccatcagtgccttttttaataaaagctctttcatctatagtttggccaccatacagtggcctcaaagcaaccatggcctacttaaaaaccaaaccaaaaataaagagtttagttgaggagaaa

SEQ ID NO: 184 - protein, 495 aa, Homo sapiens
MPMGSLQPLATLYLLGMLVASCLGRLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 185 - DNA, 32306 bp, Homo sapiens
  source 1..32306: mol_type = genomic DNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggtgagtggctcctcgctcccagccctgcggctgctgtcgcttcgcccccgcgggcgtgtgggctgcgccccagccagcccggcggcgccctgaagagggtggccggggcgcagaacactcgggccctgagcgcccgaagtgcagacgtgggagggccccacggggaatcgggcgccccccttcttcctcccttcctttccctggtcgtcttcttccccctgggtgagagcggggctcatctcctccacccggttctccatctccaaatgcacacacacaggaagactccaaaccgcgcacctcgcgcaaaaattaactagcaagaaagggcgcgatcagaggcaaggggctgcagctgctagggatccccttctttcgcacccctctcccttctcagtgcttagacgcatttggggttgagggagggagagtgggagggcccgggcctcttgcgatggaagtgcgcattttggcgaagtcggtgagaaggggtttctgccgtttgcctcccacatgaacataggagcgaagaggacgtaaaggacacaattaagtttcattttcaactcagcattcaatcagaagaatcttccggccgtgtaatttttgctgtcgtttttaatcctaaaaataagcttgctggaaactcttcctttctcgtgaccctcacgccccatcagccacttgcatacattctaaattgtacgttgaagtttttcccactttatttgggggaaccgttttaaaagtaatctggttttcgcctgaaataggaagacagtaacctccagtcaaaacatgtgcagcagaatgggatttggtgtttttcgacgccaaagaacctccacccccccacccccaccccgagcttttggaatccatttcgcttttgtaaaacgtgtgcttcgtctgtaaaaactgagcaaggaatagaaacattcgtagctctacagtgagtgcctgtcacacttcacatccatagagccttttgtgaacttttataagattcagaattcagcttgagcaccagtgacagcaattgttacatttattctagaatgctaaattaattcgattttaaactgattattagcctcggtgtgccgttcctaaaggcttgtgactttaggtatttccaagatgcccttaagtctctgcttaccactttcctccctccccgagcatcctggatgttgggactgtgaatccaggtctccgttatctaaatggttgatgttagtgtttcctgtcatcacgtttagtatgcttgttgcctttactatcattatagctaaaataatactgctttcagagatgtgttgtataatcgcataataatttcagaacgccttcatattgaaccagatatgaagtgatacagtatcatttattcaaactgcctaaaaagtaaaaagtataaaccatatactatttttaaaacaggtaggttagatttaaatcacgtagtttagaactgttggaatggtactttgagtgataggatttatgttaggccttctttagtaatagttaatatcatgtaattagcactcgtttacacacaattttaatgtgttcgaatccctagagtcatcgaattcagaatttatagtattttattttacttagttaatattaacctaaaaaaaaagcaaaatacaggcattatgcagttgagttgtgttaagtgtggactgtaaaacaggatagtatttgttataaaatatgtttttgtatgtgtttaatatatagcttcaaaaggacatgtatgaagaaagatgtctcagcacataggtagttataaaccaagggtttgagagactgactttagaatctagaatgagtaagaaagtgatggatctgtacattcatttgtattgaagtttgattttgctttcagtttcgttaattaagccccgggaatcagggacctttcctggcaccctctacagtgttagtggcctttgtggtaaaagaagttatctcagatactcatttcatgcaattacaggcaaactggaaggcaccttaatggtatgcaagatgtgaaatgaattactatgttgcattccactctgcccccactcatgtacaattatactttgtttaaaagtgcatagtttcagtggtatttattagctagcaaataaaactttaaataaataatgatggtgttgtgaactgtcttttcagtggttgaatgattcctctttgttcaaaatgagttgtttttttttttaaatcagggtacatatgattaaataaaatttttttccatcgtctaagtctagtagtaatttggcttgtttaaataaaaatgttttctcttaaagaatatattatttttaattacaagttgccattttaaggaagtaaatgcctatttaaaagtacgtatttatggggccgccccagcagtctagaggcagtgttttttaaagcattacactaaatgcctaccattaggacacttgctcatgctgtcatctgtgttttggcagatgttttatctttgtcactgagttgttcatggagatttgaaaggccaagttctaaagatgagagatagatgactttccccccaaattgatacatattctaaatccaaaataatcacgcatattcgcaaaactagatttgcatttcatagtatgaattcagctatagctagtatataggcattgcttttaatatagtaatgttctttttgtggcataattttcaatcactttccctgtcttaggtttctgcctacctttttatacaagataaacatttctattttcatatcttatctctcagtctgattttaataatatcgtcttgggttatcataaagttactttcactttgtatactagtgtagatatttcttctgtgataaaaagaacaagaataatagtgtaggatcagttttgttaaactcattgtagtactaaagtagaaaatacagagcacccaggaaatttgattctgatatacctagactaagacaatagaatctgggtttttcctctttacgattatacagtgttgaaacagaaatcaagctacacccacctctgaatatttcattccaaatccatggagcagttgcataagactcagaatctggtcaatctcatggtttgagattcagattaggagaattaatgtgcatatttgaatatatatcacacttgtaggttaaaaggtaaagcaaggggtttggctagttcaaggcctgtcaaaaccacttttttccatttcaagcaagtatgagcctatactacacaccaagcagtttgcaaagccctctttggcatatagacaaatttcagacagtatgtcagtatgtctcttaatgacttaagatgtagtatatagacagtagggatatcttgtttttattccctcatggtacttaggtgcttgttaaggccataattctggatgttattgactttaaactgccctgccctttttaattctaagttggcctcttccattcttataaggccacttttaaattcattagggggattgcgttgggtcagaactaagtacaacttgtggacaccccactctaccccagacctttattttagtgtccattgaggagagatctgacttcatcctttttgtacgtaacccagtgtagctagagactccagttcttcaagccagacactagttctaaggggcaaaagtaggtaggattgctttctggcatgtgtctttactgatgatctttgcttctttacctaagaggctgctgagacttcttactccattcctgaacatccattcccagcagataattaacttcctactctactgcaaaaaatagtggtgtgtggtttggactccctctgcttccctcccaccattactaaactgatctataacatcttagataaccttcgtctcattctcttccgtcatccagcttcagaagaaaacctactcacttttccaacgaaaggacaacctgtctaccaccttttcacttctaccatcttggagacctgctcagtcagcataattcttcagtcttgcatcttagatctttccctctccattgactccttccccgtagcttgcacacatgatcaaattttaccccatattaaaaaataaaacaaaaaacctcaagaaccaaaatgtccttgcccttgttcctgcctgcacattgtcagttgttgccttctctgctttcttacactgccaaatttattgtaaaagaatcactgccacttaatatattttaagtgcttgataaagccatcttgtttctctctcatttctcaaacatagtgtgcatcagatctcactagtccattgacagatggaaagatattctccccaaatatttaagcctcttttctctataattagcctaccatctaactagctacacatactattttggttcacaattttaattttcccctctttatctcccctgcttcccacaactaacttctgttgattactgtaagttttaggtcttctctccagttgttctttacctggactattgcagtagtctgttaacggatctccctttctcttttcccttgctgccctcagtccttcctttacactgctgtcaatgttttttttttaatgaaattaaatctgagtggtttacagttgtttatgttatcaagtctaaactcagcatgacattcaaggaagccctttagaatttttcttattcatcttttccaacctcatttcctagagctcacctaaataattctctgctttcagttcttcactttctttatctggctgtacacgttactctgcctagaaagccccttctttcccataacattcttactcatcctttaagacccagtttaaacatcaccctctaggaagatcattcatagacacattcttgtgatttttaaggaacttttctatccaaccatacaagtgacttgagattttccatgaaaatatgcagcttcatgatttcataatcaagtctatacaagtgagaagcagtggcaggttctcttgaaatacagaagaaaaatctatcttctcccacatgatttttagatttttcttctatggaatttatactttaaactttttacattcacaaggaaggtattgaatcctactttctggaccctgtgttagattcttaggataccaaatgaagacagtgtcttactttctgagcctggagagatcagttaagtaaaatagtaattacacagtagtaggtacagtgacaacatttgaacacaaggcaatgggagcaataaggaggggaagaatacaattgagtaatggtagggaaggggaacaagattgaacaactcaactgtgctttagtaggaagagcaaaagttagcaaactgtagctgccatttattgagtacttgctgtatatgtgaaaggtgatacaaaaacatgttatcttattaaatcttaacaatatctctatgaggtatataccatcactatgcacattttatagatgaggaaactgaggagcagaggtaagtaacttgcctaaggttatacagctaagaagtacatgaggtgaatcttaaactagggtcgaccccttaatgtgtgcacttaactattatacaccctatgtactcagtggggtaatagtgtataacagttaagaggctatttaggtttgaggaacagtatgtgctaaggcatggggatgagagtgcatttgcatagtcatggaattgcaataacgtcattatgattggagcttagtgtggaaatgagggagtacaggaggtggggctggaaaggatcttgaaggacctcgtttgtattccatgctaatgtacttagaatttagcctgaaaagggttgagggtgttgttaaagtatcttaggcaaaggagtgcacatttgcatttagaacagttattttgggaactgtagaaaatacatcagggccaggatggagaaaccttgaggatggaggcagggagctggttagaagggtatggcagtagttcgaggggggaggatgagtgtctaattcattcattcaacagacattggatttaggaactattaattaggttgaacctactaagaattctccatggctaaggagagggagtaatctaatgtgattcttaggtccaggtctggttcactggtagatgatagaaaatactttagcaaagtgatttaggctgagggtaagaagtgggggtacgtaagtttttggacatactgactttgaagtaactgtattacatccaagtggaagtgcacagcagacagttggctgtccaacatctgcagtttagaagaaaggcagtgcatgattcagttttttatttaaaagtttgcaggtgaagaaaaccatgggagtcagggaaaatactagggggcagagggtactgaatctgaagcccttagggacactaacatttagtgtgagtcaaggaatagatttcaacaaagaaagcaaactaggatggggtggacaaagcaggaggaaattcgtaatcaggagataccatggaggccaagagagaagagcaagaaagagactgcccactggctcaagaactgtggaaaagtcaagacagaaagtgaaccatgcatatttgatttttcacaagggattggtggtgactttggcaagagctgtagtaatgaagaagtaggggtgaatgggtggaggccacgtaacagatttaaggagtgaattggaagtgaggaaatgcttatagattgttaatttgggatgctagggagagagaaagtatgagggattttttttaagttggcaaatattcaagcaatttttaaagacacacacaaaaacaacaccttgtaaatggaatgggggatgtgggaggctgataatcaactaaggaaagtcgttgaggagactgatgggtatggaggcagaaatggaaactagctttcaggtgatgggattcatttcagaaaaggaagcaaaaaaaaaaaaaaggtgtggatagttggggttacaggtaggtttatagggaaggagtattggaagcttcaggattctcctctgaaggtttcagtttgtattaagaaatagggagagaggactgttggagagtatgggataaagggcagaagggagtaatttaagagttgttaaaaagaagcgaacatttacatgaaatatgtaaaatggtaaagattgaaggcccggaggagcagggactatgacattcttctgtttttgcctattagcaacatgaattttcttagaaatctgtaagaccaattactcttctgcccatccataaggacgttgtccatgcagaaaagataacacttgtaaccttgtattatatacttatcatcgcccatttgagaattgtaagctcctaaaagataataactatattttttcatcactatatccccacatcctatcacaatatttcatcacaggtaattcttgacaaatgttgattgcatttttaaaatttctaacctgaacttgtgtgctgtgaccaccatggattgagtcttctctgccactacaaagctcttttctagactatgatatgagatggtttgggctgatagtctatattcaccaatacttgtacagttccaatgaaggtttcaagtctaatacttttggcatttgatataaaatcattttcccattttatttgctaatttatctataactctggcattactcttggttacatttgtctgctgctatctgatttagactgctataagcatatcttgtttattcagagttttcttattcatgttaattatctgtgtttctttatttgctctctacattttaaatagtttcttcactttgctgttttatgagaagggagtaggcaaaaggaaaaaaccccaaatcagacagttttactacttaatagttttttaatgcatctttatagagattgaagttgtagttaacagctagagtgatatttttggcctgcccttatttattaattactagtgcaaaggttattcaaattgtggtttatccaggtcaattttactgttatttttactaatagcatttattctacttaaattgcttcagctataaaatgtttttattgtaacaaataatgcagtataattattgctttctgtattcctttgaaaattcattctctaaacatatgttatgaaatggtggattgttcaccaactacttcttacttacttaattaaagcctttgcaaaaagtttcataggatgactgagttcttcattcgtacctcttttttttaagaataatctcttgaaagtcaaaccatgattcatacaaatataaaatgaacatgtgtcaaagattttatttcactaattaattaacaagcaaactagcaagaaggcaaaacccttttaaaagaaaattggaaaaacagacacatttataggcaatcaagaatgcctaaatgaatttgctaataaatgcaaaattggtcaatatcctcagggcagtaaaatgtagtcttttggaatcctctcttccaccaggtggaaatcaattgcagaacaagatttatttttatctgtatggacagcaatccatttgaattactctcagttttctacaacttatgaaactgtaaaacagcccagtcaaagtcagtgaaaggcacaggcttctatagagtcagataattccaaagcgtctgttagacattgcccagcacttgactgaaaggatacccagtagtctttgtatccctttcaaaatcttcaccatattttctgatactttccttcctttataatttgaacatcttcacccctttattcttctctggtatactatgtcctctctccctcagtgtttctctctcaggggaaataattcacaattccaaagttttaaagaatgcaacggaagtccaggttttgccttgggcttcctagtatttgggtggctagaaatgtagaaaactgggaagaggtgagctgtggatgcccacaatataggttcagactgcaatttcccagaaatataacaatttggactagtcaaagagggcccatatcattacattaaaatgccagattatctagtttttatagtatcaccctacatttttacagcatagtattgtttagagagcacttgccgctatgttttccatgttaatgctcaacacagttctgtgaagtagcccggtgtttgtttttgatcctcctacttaagagatcctcctattttagagagtaaaccaaggcatagaaaagtgagttgcttaaggcagcatattaaaaaggggcagaaatagtatttttactcaggtgttttgaacattgtccagtacctatattccagtatgcatccttgcattcaattcatggggtatttattgagtaaacatagtgttcttacaatagaaacattctatgcctctgatttttattccagtttatgtagaagtaaatcttaagtgtgaactattaacaaagttgatattttatttatatttgttagtaatttgtgttttgtttttgtttatgttttgaggggaaggccaagtagctacttaggtaaaagagttgctgagtggctgaagaatatggaagacaactacaattcctacacattcttgtacattttagttgaacaatgagtgattacatttatttacccagtgcctcttctataaggcaacaactggtagtttatcctattgagaagcgtagataggatgcttattagcagtaactgctcttggtttcaacttgcatcttacttagctttttcaccgttttgtggtttcttggaggaagaatacccataatatacatttggagactgttgttctgtagtgtcaatgaaatgtgggggtgggaaaatgtcattcaagactcccatacaaaatgtctattgctgcctatattttgctatgggaaagtagccacagataatgttttttttttcctcattagtattttaagattttccatcctagtggaaagatatgatttgattcatcctatttactttgtatattaaagtacagtagaacctgccactttttttggaaatgcagcataaggataaagataaatttcatatcagttcagcaagttctatttagcagtgtgttgaagttgagactgaataaaatatttggtttggttttctgttcaaaattttaccttgataaggacaatatttttctacatatatcagtaggcagtaatgattacttcaaagcttccaaagccagatactacacctgcatgttccaacatagttgctgaatttattcccaagatgcatgtaatgtatactttgtattattgagaatgaataaagaaaagtcataatgatgccttccagctgtgcaagttaatattaaaatataatttgtttgcatatttcacctaataggtcttcttcattgctatactgtttacttaagtgaacaatggaaatgttgctgtttatcttaaggatttgtaacatgcctaagatcttacagtacagacttctataattaatgaaacatttttctttttcctttccaggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttggtaagacttctatgaaagcttctttttttatttgtcctggtgcaacctgatcctctttcgagaggaggccaaatggggataggtactcctttgaatcaaaaagcaggctgttattaataaatgttgatgtaatgtttagcaagtttaagattgtgatttctatcctatttgttaatctactcttcatggtaaaacacatttactatttaatttgtattacttgttaatttgtatattgtgtggttctcaaacttttggtctaaggaccattttatttggctgattggtcctggggtaatgtactcttcatttctgtttgctgccatccaagtgctttctttcctgtgactaattttcaagtctctttccaattattcagccttctgttttttgtgttttttttttttttttttttttttttttaaccttacactttgaccaagggatgacaagtcccagccatgctccctctggagagaatagaggagagtcaggagattaaatcacgttatcatggatgatttcttcagattttctgtgcttagccttactttggtcttctttttgttggccaaaatatgagagaactagagattcaaattcatttattcaaattcagacttgatgatagcaccatgattctgccctttttatcacaaacttgggttgctgaggtgtagaaggtagtaaaattagctgaagtaaatagtctcctctccttgctgtttcctgctgtgctagctgagttcttcaaccccatatgagtatgttctttctgttttgaaatctctatttagaattacctgtcatattaccgatcatggcttaaccacattttgagaaccttaactctagataggttcagttctgttcagttggatcaactatttgcctagcttttataaaataatctactaccagattaggaatggggtgtttggtttgatgaaatgtttgcacattacttaggattgagaatcaggaggtctgagttacagcccctgttcagcaacttactattcacctgtgtgatcttgagaaggtgcttaaccgagtccaagttacctcatgtccgaaatggagatgacaatagatacttgcttttatgtattctacagtctaatgagcataaaccaaagtgtgttgtaaatgttagttgttttcctgggatgtttattttatgaagtaaggtatcaagttgttgaagttagcctagagcattgtgagagggatttgtgtgcagtgaggaatgggcaagggatggctccgagtaggcaaggagaatttgaaaggtctttctgctcagaaatctctcttacattgtagcttttactattgatattaatttgggcaaaggagctttttttgttgttgttaagtatacctttaatgaagggtgtaatcagtcagtcatatccactgcaactaactggtattagatcatctaatacctagttgaaatttttctgaggtaatgtacaattttgtggaaatgaaagaataagatatgggggaacagtaacttttggggggtaatgcaggtcactgagaggtaaactggaaaactcaaaatatgagatttgaaaggccagaagatgaaatgaagatggaagagaaaggaatgccaggatctgagataaaaacagtttattattttgcgtgtgattgaaagactgttttatctttgtttgcaaaataccctataaaaataaaaactagacttattttgggagccattaaaaggagaaagtattgttttattatagagaatgtagaaaattctaccagaggtttgagacatttggctttgcatccattttaattttttgaaataattattgatttacaggacgattcaaagatagagagcttccatgtgcccttcacccagtccccccaatggtttcctcttatgtgacacaatatgaaaatcaggaagttcacaatggtgcagtgtgtgttaatggttcgctctcattttatcacgtgtttatttgcgtaactacctacacaatcaggacatagacctatccatcataacaaacatctccctcatgcttattcttactagtcataccgccacctcccactgtccctaacctggcaagcaccaatttgtcctccatatctataattttgtcatttcaacaatgctatataaatggaatgatacaagatgtgaccttttgagaagggctttttcactaagcacaatgcccttgagatctatccaagtttttgcatctatcaatagttcttttttccttacttttttttttgctgagtagtagtccatagtatgaatatatcacagtttgtttaaccacttacctatagtaggacattttggttgtttccaagtttgggctgttacaaatgaaactgctctgaacaattgtgtacaggttcttgtgtagacacagttttcatttctctgggataaatgctcagaagtatggttactgggctgtatggtaagtatatgcttagtttttaaagaaactgccagacttttccagagtggctatttcattttacatttccatcaacaatgtaagagtgatctggtttctccacatcctcaccagtatctggtgccactatgttgtaaccatttttaactgaaaaaaacaacaaacaaacaaaaaccagttaaaaagttatctgcacaaaaaggttaaatggggcaggactcttactatggagtataagttcttttctaataagaatacttacttgtcacggactttgaggatttaagcattagaaaccatttttactatgtcgtgatttttgcaaatacaggcataaatgagaaacagaatttgctcaatagagaagtgaattctacttaatagaatgcagataatagtgaccattctttgtgcctttttaaattttttggaggactaggactgaatacatggaaacactataagatatagttttatatacctctgtgcctatgtatgatatagtccattagaaggagcactggacttgaaattagaaagctgttttctagacttcactgtttaatagcttaagaaaagttggataaaaaactcaacctatctgtccctcaaattcttcttagtacagtaaagatggcatcaccatcatggaaatgtttgagatataaataatacctctcttacttaacaaaattttactaagtgcctaccatgtgccagtcaccgtactaggcatcacagattctgtgatgaataagacattattgctgatttcaaggaagtggttgcagtacaaatatgaagagtgaagtgacctaacatttatttgcacatgtgccacatactttatgtaagtcgtgtcctgttatcctctcaagaactctaggagatagatggagttcttaccatttaacaaatagggaagcaaaagagattcaaggtattgttcaaggtcagagtaatagccaagaattaaacctagatctctttggcatcaaaacccagtatttttaccaccgtaatatgtggttcatgtgaaaacaccttgtaaataactaaatactgaatgcaaatactagtaatcataaaatttatcattaatatttctgttctattagcaatacaaaaacttgaaaacttaaagtttatttttctctaagctattagagttttgtttagaaaggtcagttcaagttccgcagtgttgccaattccattattatcaacaggacatatggtctgtttataatgaagacatgaaggcattgcaagatctttgtattagttttctagtgctgcataacaactacaaatatagcagcttaaaacactacccattttttagctcaaagttttgtaggtggcaagcctggtcatggcatgactgggttctctgatcaaagtcttaaaagactaaaatcaaggtgttgcccagggcatactttatttggagtttggggtcttgttctaggctcacataattgtgacagaattcaattccttgtgattgtaagactggggtccctgtttctttgctaagtatctgccaaggaattgtagcagctcctagagattgccctcatttcttgccccatggccccttgcatatttaaagccagcaaaggagaatcttcctcttgttgaatccctctcacatattgagtttattttgccaggaagaacccagacccttttaagagaccacttggttaggtcagctctctctccctcaaaaataatctctctttcttaaagtcagctgatttgggatcttaattacatcagcaaactcccttttgctgggtgatataatcatgaaaatgaaatccaatacattcacagtcctagtctcccacacacaaggggagggaattataaaagatgcatggctagggcaggggttttgggaccaccttagaattctgggtttgattaaataaataatgggactgtagcctagcaagtcgacacatcagaaaagccatcacaatcctgaaacatcaagtaaaaatttggtttgcattttaggattgtaaatgttactgtggtgtatgcgtgtgtgtgtgtgtgtaacctggtgttccctatgtttgtgggagtttgaaggagacactttgttgataggaatggtgtcttcacttttttaggttgtctcatttttgtacagtgataagacaaatgaggtcctgggttttaataacacttcagcttgaaagcaaaaattaaacacttattcattgactacacccttgtaatatcacttctttgcctttccactagcaagaagttcattttcgtggaagccattctgcctgtccaggaattggaggagagtgatagacacagttgtcagccatagcttgggtagaataaggatgtgaatgtccttggcttatctttattaatcttgtgatggaaaaatatctgacattgttcttagtccattttaagcttaatttatgttcttagtggcatagaaattcagagctgaaagaagtatcatgtctcactctccccttgaggaacaagagttaacgtcatctgacagtactaccataacaaatcaatagcttaataggcatataagtggctttatataaaatgttggtttttttcccccagcatctcagttggttcttaaatatctaattccatgatcctcaaacttttcccactgtaacaaattagagagaggaggaacatgttcatcagtgactgtagttcaaatacagcagaaatgtgcagcagtgatttcaaactgagagaatcctggatgcccttctccattgtgtgccccccccccccacccgccccacatatcagggaacaatttaaaatcctggcacaataatgagaagggagagtgacaaactgataagttccagttaagaatcacttagacaggccaggtgtggcagcccacgcctgtaataccagcactttgggaggctgaggcaggtggatcacctgaggtcaggaatttgagaccagcctggccaacatgataaaaccctgtctctaccaaaattacaaaatttagctgggcatggtggcgcctgcctataatcccagctacttgggaggctgagacaggagaattgcttgaacccgggaggtggaggttgcagtgagctgagaccacaccactgcactccagcctgggcaacagagtgagactccatctcaaaaataaaaaaaaaagaatcacttagacagtattttttgttctgtaatcttcctctctgtcattgaaatgtctcatatttcttgtttcacatagaatctgggagtcatcaagtagtcatctagtctatctcatcatctaatttacgaattatttctatcacaccccaagtagctggcgattcagattttttactgaaagcttgacatgatgagacattattacttcccagaggatcttggtctgttatcaggcaatttcagttcttccttaaatcaagctaaaacttgctactattctcactcactgggagggctgtagtatttctctcaagtttatacctctctgtgatgaaaatctctgttcttttagccattgctcatggtttcgggatccccattctagtcatcttcctttggagcaattccctgttagatcttcaaatgtgatgagtaaaactgagctcaggactgcgtttgttggtcacacccttctcccttccaaatgactgctgctgtaactttctgacaggtcctgcttccctgtaatccattctccatgtggctgccaaagaagatttgaaataagtcaggttatttttcttccctatttaaagccttccagccgcttgtcattgctcttagaataaaatccaaactctcccaacaagcttttaagactctacatggtcttgccccagcaaactcttcccatttgatctcatcactgaattccagtcacaatggccatcctttggtttctcaaagttgccgttcctttggcatctcttcctccctgtcttcacaatgtaggctccttcccatcctaggcttagtttcgttcctctccctagaggcctttccttactgcctcagcttctcccactctgcacactcagggtcctctgcattgttcactgctggccttcaagagcctagaggagttcctccccatggtgggctttcaataagtgtttgtggaataagtgaaaaatgagtggtcacaccaggatcgaatgcacctcttacttttgttaatatattcaaattaatattaatatattaatattctattaatatatcctaaaattttgttgtgtataggggcagggaccaatgaaagaacatttcattgtcagctcatcttgactatgagatcagatattggctatattttgaaggtagagtcagcaggatttgctgtttaactagagtgggatgtgggagaaatcagggagtcaggtataactccaaggttttcagcctcaagcaactgaagaaatggggtcaccacgactgaaatgaggaaaatgagagggggagtttagtgggggatggggtgatgaaaatatcaggattcattgtggaacatgaacagttaagattaactgaagagcctgaattcactgttcctcagtttctacaactataaggaggggttaataatttctcacatcataattgtgaggatttgaggagttgaggtacacaattaaaaacaaaagcacaggggaagtaaggaaaaatacatcaattaagcatggctattagaccatacagccttaccaaatccattggggattagatacaaaaatccaagcatgatctccaaaattttttcctggtagggttttaggctatactgaagtgacttttctttggcataagaagatattcagttatacagttggaaataaaggtattgatttggagtatccaaaaacatctctcagtagagatccacaaccaaagaagcataaaaaaaagtcttccattcactcgcaaaactgtcttatgaccattgcaaccctcaacagcaaacatatggagtctctactacatatagaggaaaatgctagattctgaaaagctaatatctataaaacagagtttatgatgttgttatattctggggattgatgtaagcattttaccaggacttattatagcattataagcttaacacaagaaatatgtggctatcattatggagtgaaaggctggaaaattcctcacctgtgacctgagagatagtgcgtggtagaatttgaaagaagtgttgatttcagaggaatgctagtgcttgcttagggcagtattcagaagaactcttccaatcacacagccccttgtacaggcaaactccggaatactttcagtacatttacttggctttattgttagagcagaaacttcacagctaaaattatggtcttaggggtttataagatgcaattaaacttaatttttaaaagttctcacaaatcttatttgaagtcttaatatcttaatttcctttatataggatatgtggatatatttttatgataaaagtaacatgtgaccaacttaataacagcttcgttaatatcctaaagccactcatcttactttacatataaacagtccttctgaagcctaattatgcatggataattgcagagtgagtttgggaaaagaccaaccgggcacatctctctccattttaaccacaatgagaagagagtacctagtggtgcatcttcctcgcttaggttccctgagtctgtctttacaggaagacttcattgttacttgaaggtacattcttggaactttacacacccagccactcaacacatgaatacatacttattttagttaactgagtacttagtgagtgctaagctttgttttaagccctttgcatgtagtttattaactttgttaatagttacaacagtccttcaagatacatgtactgtgttatactagtgtaacagttgaggacaccaaagaacaaagaggtgttgacacgtggtcacggttccatagagtgttaagttagagttgggttcaaaccctggcagtgtggccacagagccttgttctcagccactgcactgcactacctcctcccgtgaaacataagaaaaatgtgagaaatgcttaagtaagtgtagtttttattcataaataaaatttacataagtacattatgtgtaatttgttttatgtatatgtgtgtatgtataaataaatacatgtaaaaataaggccacagttttaatttttttccatctctataataaagcatgtattatagaccattagcagaatttaaagtgttatagtaaatattaattgtgacttttgttttcttcttccccagtttcatggttttctccaaatgaaaatattcttattgttattttcccaatttttgctatactcctgttctggggacagtttggtattaaaagtaagtattatttctacttttcatttatgtttcagtgatgatatagttatttctacgagacattgtcagcgaaatatttaaagttgtactaggaaaagtgctattatgataaatatgagtatgtaatttgaatactactagtctccttgaagtatatgttgtcgcccacattttgctgcagttcacttttaattcctaagaaggttgttttcacttggtgtttttttaatctcttaagaatgaatagtaggaatattagtaccaacaccttaaactcatgtcacattttaatattcacagaacatctacacacacattatgttattaggtaaacaggtggtgacagcctgcattagttttaaggtaggacgttatactttggagcatttagattcccctctttttattttcccagtttgattttctctgtgtacacgtgttcacccttggaaaagtccagtcggaactatgttttgtcatcctctgcgtgcagttctgcagcctctaaagaagcagccaccagagagttaggttctttgatcttgctttcctataatagtaacgtaaccagacttctgaaggcagatcttgatgctgcattagatttagcttcaacaacacagaattgtcattactaggcaaataggtaatatgcattacggttaatgtttaatcaaccatattttcatattttggtaaagaaaatttacaaaattaatgaagttctgaggtgacagtctaaacttttaagctttttaaatacaagatttattccttctttccttgtagcatcctgaagaccagtaaacatttatataagcaaggaataaatactgcttatttaatttattctgcaacctttaaacacacaaaagctagtaaactattgcagtggattgccctgttgtatattttatgaattttacttttactcagcagtttaagctgtctatatctatggtggtgtataaacatggaagggagatgactgattgattatatgttttaagcgcttttctcagtgtatggcattctggaaatgcttagtgatttcagaaatgttctcaacttttgtctgaaaggaaaaaagggggaagaaaggggtggcagtggcaactgtcaagacattttataacttttactttcaagatagtgtctagacttcttttggaaattttcttataatctcttagttttttatgtcaagaaaaggactggtgtagcattaagacctattctggcatcaatgatattagggaaagcttttaaccatattggcagagccagactttaagggctaagtcatatgacttgggcagggaacagcctttttcaaagatcatgaaattatttcacatagtgcgattttttgagtttggcttgatggatgtttgctgatccagacgttatcattggtgacattatttttagtgaaatgaaccaggagtgagtttgttcactgttggctgtatttttacaaaatgagctttacaatattttttctgacttaaaaaagtcatacatatcactttagaacacctcgtacaaagtagggagttatttaaaaaaaaaaaaaaaaaactcatctgtagtccccaaacctagaaataaactattggtatattctggtgaatttcctgtttccctttctgtctttttctattcatttttgttatcttttttttaaaaaaaggtaaccataaatggactcgtacagcttatatgcttgtacgttctgatgttcccccatgtcttcagaaatcttgttatagctgcctgtgttctacttttgtggatgcactataatttacttaactctcatcttgatggattttaaggatgtttccaatttttttgatactatgaaaataactctgctaaagatatgaagcctggaattggcagagatattttaagactctagatacatactggcaaaatgttttccagaaacgtttgtatcaactactattgcagatgagaatacctgtctttcacctcagttataattctgatattataacgctaaaatctctgcaaatttgatgggtgaaaaagcatcttattgtaattgtaatttttgtgttagtaaggtggaatgttcttttttatattttttgtactggtcatattttaaggaacgtgcttataaacctaaagaaatattttggtgggaattttttgttttggctcatcttgaaacaggtagatgtgtgtgtatgtgcatggaagaggtatgtctatacatgattcacccagcctgcgctcacatttaaaggtgttgatgataatagtagctaaccatttgtggagctcttgctctgcttgacaggttctgtgcaaagtactctatatctgaaatgacatttatttctcccagaaactctatgggcatagacactgttgttattcccgttttgtagataagaaacaggcacagatagattaggcaatttgcccgcaatcacccagccgtttcctgatagtactgggatttgaactagtactgtttaccactgcactgtactgcctccccgttttgcatttattttgaggattttatttccatgaagggtgaacctatatctaagcacataatactgagtagctaaaacttattaggagagcagaatgttgacctgatttgtttacttattctgcaaatacctagtgacagggtgcctactgattgctggacaccaagctacatgcctgaaatgtgggtgtgatgtgcatattgccctggttttgtagcacccacatttaagccagggagacacgaatgtatgtgcatcccatgccttgtcacatctttgagatagtcatggatccaaatctaacttttctgttagtgcctctgttgatggcctgagcattccaccaaatagaaatagaaagcacctctattctccacctgcttagatgtatattttttagaatccagtatggtaaatcttctaaagcattcataataactcagacccatgacttttattttatagatttctagttctgcttaacttttcctgattaccatttatggtctgtacttggctacaggggatttctctcagtggtttttccagctctgcatgtgagtctttgtggtccaagacaaacctggacctttaccaggctaaactttcagtaaagacagcaggttatgccctcatttgctcactctgaggagaaagagtttctttataccagagctgtatcttgaaagatgtctcaggatgccattggtcctactgaggaagaagcctgagaagactcttaaactcccagagcccagccagcacttggtgagccctggaccacgtttcaaagataaaggcctcttacagggaaatgttcctaaataccttcacctttagcttagctttaacttaggaacttttaagcagaatctctatgcttagcaaacagctcagagattgctagaatcaaacaccaaggcttaactgatagtattgaatttcagacgctttgtttgtgttctgaaaactacaccaactcacagtttgccactctactgacaattaagttcctggctgattttcaggattcttctttctcactctgatatcattttaagtgctgtccacctagtcctccagttcctgcaggattaaggtccaactgtatgtaaagaactggctaacattttgaaattctttgagataggcctatctgttctttctttgcctttgtaactttgttttatacaggcaatactggcttcgcacaaaaccgcaagaccataaacatgaccatgtaagctgaaattctgcaaaacaatcttcatgatgaatgggaaaaactattattatattgttccatgacctttaaaattttttttgttgttaaaaccttaaaaactctgttattatcaataacaacggcattgggaaatgaaaaatagtaaagctagtatttagtatgtggtaaattaaatcattagaaacatcgagaatgaaagtgtgttatcaagagtagtttgaacaacacttgtgtgttcttcttgcataactttggatacagagcaagcatcttctctatggcttggtgaattgtcatacccctttctaagtttggatcggtttctaccgttttatcctttgcactctcaatgtcatgaaagaattcctttaatgtttttttgaatgtttaaagtttattttattgccagtcacttcctctgggacatctttattctttttgttacatcctcctcactttcctcatttatatcagtaagtccaccttcgctgagttcttctggcttcatctctagagtttcttgcattgcagtaatgtcgacattcgcacaatcagctatttcttctcttactccatttatggtctattcaaatttcactcctagcatttgtcagtttttatttctttactctttcatctttcttgcctagtttcctcttttgatttatccattataaaatgtcacatgggtttatcactgggaaacaaggaggtaacaaaactccatactttgctgtctgtgcatgacctaaatatcagatgtacagtgactagtcaccaacagtcttcaaaagaagtgacatggttgatcactgatcatgatgggacatctgctatttacatagttatttttgaactgaagaaatagcagtgaagttgtactttatgcagttactcagttaatatattgtggtaattgaaatttggactgtttatgagggatttatttgattaaaccatggtaactggaattcatatcagaatagtgcaaagtgaggactgctgtacttgattccaaatttaaaactgtattctaggcatctttaatttttttttttcaactttcccctccctgctcaaaattactccacagagattttggttacggctcagatgtctttgagtagatgttcctgtagaaatcattgttagaaaattcaaggaaaaaagttctcattacactaccttgagatcctaccaagtccttgagttttgacttgaggacagcttaataatgaaagtaattcagccatgaaagcatatttaaaataagtaatggaacaggaaatttcttcatagattagaaaaattattctgaaaaagtgaagacatagcctatcttggactaggaaatttccatccaagaaagtaaaatatacaataaatattatcacaaagaatcattctgtgacctggttgatggcccctggtaataagactttgttattaagtttactgtgaattctttttttttttttttttttttttttttttttttgagacggagtctcgctctgttgcccaggctggagtgcagtggcgtgatctcggctcactgcaacctccgcctcccaggttcaagcgattcttctgtctcagcctcccgagtagctacaggctactggcgtgcaccaccatgcctgggtaatttttgtatttttagtagtgatggggtttcaccatattggccaggctggtctcgaactcctgacctcgtgatccgtctgtctcggcctcccaaagtgctgagattacaggcattagccaccatgcatggccccttacagtgaattctgttcaataatcttgaactcccagtgctttcccttggtcctgtccatataatgatcacattctgttattaaataatgtgcttatgtgctgattttattgtaggaggaattgaacaaatctatagctcagcattttactcttttgtagaaatcccttgaccctttccaaatccagacatcacaaccttctccatatactcaaaaattgtgaaatccacaaggcagggaaagttgataatgaaaaaaaggaagtttggtctgttggacatcctcttctttcatcttttatgttcaaagtacttgatgcacaaagcctggactcttttatctctgtcctatagactgatgtctagtcagtattcattgtgacaaaattgtttcattagagcaagttggatagtgtatggagattattataaaatgatttaacttgtcttctgtcaagtaattttataattggtattaatgttcatttttttccagtaccagtttcagaagctttcatttgtgtatgcatgtgtgtgttaaataagtgtattagatacttgaaaaatagtaatttaaatttaaacaatttaaaaaatgaaattgtaattttatgtgtaaaggtgtctgatgtgctttattctgcactgaaaacaatattcatttacagctcaacagacaccaaatatattctaaaattactttcctagagttatcagaaaacagcactgtaataatgaaatcaaacccatctttctttatgatttattcttagtctgatacgcatcagcctggtaagccttcctgtctctctgcttacttaccaatcactccaaatgtcatgtctttgggcaggcattaaattcttgggttttgggttttgttggatggactgcagtctgtgtgagcctatatgggtgtgtcaaatccagtctttggggtgtcatggaaacttagcatgatagacttgattttatccccaagttgacttggtaatttcattagatttcatcagtcacaacctgcattttatcttgtatgtgctgtctattggtcacaaaatcagaaaaccttcttgtccattcataacattagctgttttttcaggtggctagagggacatgtcattgcttcatctgcatgaatttgaaagattaaatgcataaaggaattttcttagagtagagtaggccttcaccatctctttaactgggaaagaagttttgggagtaacatactcatcactcacacccccctcccctcaaacacacacactcacccattagaatgtaagggccttgaggcaggccttgtgtttgcttttcgtgttcactgtcagcatttagaatagtgatagtcatatagatggatagtgctcaatcaattattgtcaaataaagtaatctacttgttccttgatttagactagcaaaaggggctggtacattgtaggtacacagtaaatatttgtagaacagatgaatgaaccaacccaacagatttcttagaagtagcctgcttttggttactaattattttatagaacatataaaagaaaattttagaatacctaatttgttcacaaaaatgttatattgtctcccctgatacttggtattcctgtgtcaggctgagcactaaggttatacattttttgttaagcatgtaattactgttatttctcatgtactttattttgtgtattgatttcagtagttgtagctgagggtaatccttaagagcatgcagattttaaaaattacatagattgccttgccaagccgtctgaaagtcatactagaaaatttgataagagcctgctgttaacaacacattgagttatttttatcttaatgctaagtggagaattacttgaatttattttttttgctgcatttaatgttttgatacattcaataaataaggataaatccacttactgaaggacaagaaaagatcaataagagatccttgaggttattgggttctatggcatctattatatcaaattccttgccctttcactatactgagaaatttgacgactgggctagcaacaagatacctaccccacattagctgtggtacctgctgaaatgtgaccatgtgagcaaagaaaatgagccaaaagaaactctttctgaagattcactgaagaaaacctggagatactctttcattagtagaccccacagagacaggtggagtgtccgttatccagaatgcctgggaccagaagtgttttggatttcagattttttcacacttgggatcctcaacatgtgtatttgttggaaatggctacttaatttaaggaaaagtttaaggtggcctggaaaataagtagtagttgaactaatcaacaggaaccaaaactactttcaatatatagtgtacttatactcaaagagagacggaccattttatggtggaaccctgtctatggtagtattttgtgatgttttatttttgttgctattgtttgcactgttttctcctataacagctcttctaagccttaagaggataaattttatatgagattcaaactcttatttttgttaagaaggatgtaagtcaccaggcatggtggctcacacctgtaatcccagcactttgggaggccgtggcaggtggatcatgaggtaaggagctcgagatcagcctggccaacaatgtgaaaccccgtctctactaaaaaaaaaaaaaaaagaattcaaaaattagccaggcctggtggcgcatgcctgtagtcccagctactcgggaggctgaggcaggagaatcattgaacctgggaggcagagtttgcagtgagccgagattgtgccactgcactccagcctgggtaacggagtgagagactctgtctccaaaaaaaaaaaaaaagaaaagaaaaagaaaaatatagaagaagaacataagtcaacctttttccatgaaattatactgtactctaaggcaaattcttgttgcttgatcttagtagcttttttatgcattgacttaaacctggaagagtttcttatgaatgtttgattgactgtgaggtgcatttgaaacagcttctactttataaccctttgcagaactttcaagctctatttagataacaatatatatgggattaaaatggaaaatgtgacatatgtctagagaaagttcctttttctgtgtatgtgccttttgtgacttatacactcagtttcattctagccagcatttgagcctagaaaatagggaagttatgaaaactctgtgccttataagaaggcaaggctatgactgagtattagacacataagtccagggctgggcggaagtaatgaatcaaataaaaacttggggaacttgccataggatgttttctgattaatatgggtttctcttctctgattaatgtggatttcagttactcaactgtgtaactgaaatccacattaataaattggttatttattgcttaggacttatctccccataaagagactaaaattgaggttataaattatgggtagatagaaattccagaaatatttaggtacctgtcatataccttaaacatagatttttatcacaatcatttaggggcttatttattgcttttactttatttaatgtttgtcactgtagaagaaaaaaaactaaatgctaaatataacatttaaaatattttccccttcatgacagccaccaattgattactgtcatggagactatctctatgtggatgacacacagcagcatttcaatcacacgctgtttctttccgctactcagtttgcttttaggttgccttaaacaacttccttggtgaaaaaatcaacttcaatattaaccaaaatttaaaagattcatatatagtaaaagaactaatattcgcagattgacccattccattctttcagagaaggtatgagatacttgacagtggagctagcagcagaaacaataacatgtatgatgaatcccattgagtctttgcagtttatttttattaaaatattttaattgaacaatttaagctttttttcttcatcaggatttcacaagggtgtaatctgcccagttttattctgtcatttttttcaaaacaaaataactagatttccagattgtttactaattttttataaggtaggacaaaattctctttttctcaatattgttaattaacccattatttcccattagtattatgtacatcagtgttgtgttgtcacttggagtggttttgctcatcgggggcattgacaatgtctgaaaacagtttgattaacataactggcatctagttactagcatctagtatgtagaggccagggatgctcctcaacattctatattatgcagagcagtgtcccccctccccccaaaaacttatctagctcaatttattagtaatacatcaaccgagaaagactgatgtatagaacctcatttgttagtgtaggaaaataaggtgtcacctataaattcaccatccatatttatactgccgtgacgttatccatttgcttatgaaagagatgtgaggtgacttgatgatattaaggagttgttcctcataagttattacattatagtacttctgtcagtttgtctctgtaccttacaagttattaaaatggcttcacttgtgattgagttcatataattctttgtttttcttttttaagcacttaaatatagatccggtggtatggatgagaaaacaattgctttacttgttgctggactagtgatcactgtcattgtcattgttggagccattcttttcgtcccaggtaagatgtgcagttcctaggcaggaacgcaggaggtagatgagtgcattccaaggtgaggagggctgcattagtttcctagggctgttggaacagatgaccacaaactgggtggcttaaacaatagcaattccctttccatgctggaggccataagtctgaaatcaggacgtcatcagggccacactccctctcaaggctgtatggaagaatccatttcttggctcttctagcttctggaatttgctgaagaatgacagtgtttatcattccttagtttgcggcagcataactccaccctctgcctctctggtcacattgcctcctccttcctctatgtctttgcctctgtgtttttgttaaatctccctcagcctctctcttatgagcacatttgtcattggaattagagcccactcatataatccagggtaagctcctcctttcagatccttaacttcatcatatcttttgccatataatgcagtattcactcttttgctgtattaggtaatattcacaggttttagggattaggaggtagaaataattttagggccatcattcaactcactacagaggcaaacttcattggccaagacatgaaagtagaatgaatgtgggaaccaagtctggaaattggcagttgcatttggaggatgggaatgtgagtgggaaatgaggatatgtagcacatgtaaagaaaagcaaaggaaggctgggactcaacctaataactatagcacaccatcgtttgtggagaagaatgcagtgcaggtgtaatatttaggaacatgggcttggcagctctattggtatctgaagttagtgaagacagcaggaagaaggaggtgaattcagaagacaacttgaaggaacaattgatttgactttggggcatactgaatacagcagatgaagggaacagtaaaagatgaatacacagtttctcactggaattctgtaaactagttaagaaggaggcagagccaattcaaggtttcagcatgttgaaacttaaatgttgagatttagatgtcttaagtttggtcttaatcaaaaaatagtcactaattttgtgtgaggttttcagggaaacgtatatttttaaaattttcacagttgtcaagcacagaatttagattagtacatttaaaaagtatgtaggaaaatattttaaatgtttttatttattgacaggtgaatattcattaaagaatgctactggccttggtttaattgtgacttctacagggatattaatattacttcactactatgtgtttagtacaggtgagttttcattgttacagtatgattttgtctacctttttcatttacaaaacagcagttttggtgaaaatgctcatataaattttttacaactcaataagagtaggtttattaaaagatttttcatgctattcttagtgacattttcccattcatattaattttaaggttattctaggttagctgttttgtaaaaagtgactttcatatgtatttgatgccaatcagcataattttaaattatgccatataacttcttaatgattattttcatattctaatttcagttttttgaagttataagtggatgttaaacgcagtctttctcttcttttgctaagcttctgcttctgcattaagaagactggttctataaaatggaattatgaatcacaaatagtagaaacatatttgttttaattattagcaaaccttaatactgtagttttaagagatggtatggaaatccaaactataatcagtatcattttcactgcattttgaaagtagatcactaccatatttagttattactattaaagagtcacatttaacacttggtcttagagcctatctctggctcgttgtacattggaaaggtattagtttgttataaaatgacatctagagatagagagcatgatgtttacagtcaggtatttgcataatggttccctcagcaagagcaattccatgtgcatgtggagaagacattcataatagcca

SEQ ID NO: 186 - DNA, 5292 bp, Homo sapiens
  source 1..5292: mol_type = genomic DNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 187 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 188 - DNA, 30154 bp, Homo sapiens
  source 1..30154: mol_type = genomic DNA
agtgtcttccactcggtgcgtctctctaggagccgcgcgggaaggatgctggtccgcaggggcgcgcgcgcagggcccaggatgccgcggggctggaccgcgctttgcttgctgagtttgctgcgtgagtaccgcccgcgcgccgcggccgcttggcttcgccgcggggagggtggaggctttctgggaggctgaacagcagagcagagtctcacggagggaagggacccctgcccaacccacgcactgccgcccacagctgcttccccccggggccagcgcctcacctgggagctgacgggggtgggaggggaagggaaggccatcacccccgcgagtgtgcgttagccgaggtgtgaatcggtcagcacgactggttccaatggactgagataaagcgctttggagatgccagggtctcttctggttgccagaggcgcggagtgcgaagttgcagccagaaccgggagactggggaggagagaagcaggaaaagttttgtggctcttggttgattcagaaagtatagacacggagcggattgctgggaaggggccggtgtgcccaccttgcacagggactgggagagccagaagtggacattaaggaattcgagggaagcgaatcagggatgaggctccaggtcccaggccaggggtgttcggaatgaacagtccgtgaaaaggaaaaacaaaaccaaacaaccaaccaaacaaaaaccttgcatttaagattgggaagctgagtttgaattcccacttcagcaaccccaccgcggaggaggtagaagtagacagaatttgctctgcgcccagcctcgctcgccttaccaaaaaccgtcacccctgcacaaatttcaaaacttgtcaccttttggagttttcttcctctgcccctgctccaagttctaaagctagtggatgggttagaacactgcccccactcacctacacatatccttactcttaaggtcttgctctcacttcggttaaaaaaaaaataggcacccggtaaatatttatactcgattactttccttggggaatgacattctccacctcatctctttgcacttggaaggtttgaaacttttgaaactttgtacattgctctcccttttgcttacagcactctagggctcccaattctgtactctcactacatcctaagggccacccactgtggccctgcagagtgacctggactagacctctttgttcccagtctcctctgttgtccatctttgggtccgcccagctggggacagctgcccctgggctggaggaagctctgagagaagttttgggttgccctgtttggcagtcctggatgtgtgctttggggcaaggaaaagctatcaatgaggtttcaaaattctagagtgggtgctagactaggccctcataacaggagtgagtcggccgggcgcagtggctcacgcttgcaatcccagcactttgggaggccgaggcaggcagatcatgaggtcaagagatcgagaccatcctggccaacatggtgaaaccccatctctactaaaattacaaaaattagctgggcatggtggcacacccctatagtcccagctactcgggaggctgaggcaggagaatcccttgaacccaggaggcggaggttgcagcgagccaagatcctggcactgcactccagcctgggagacagagcgagactccttctcaaaacaaacaaacaaacaaaaaacaaaaaacaaaaaaacaggggtgagttggccagtggtgcagattccaatcagatgctccattctcctttggctacacagcttgtgatgacctggtcctcagccgaatgaggtccacccttttcttatttcctcagctggtaagtgccagtctcgccttggcctgccgaggttggggttgggggagcaacacaaccttggcaaaactttggactgatatgctgagaatatcccagcttttggtggggagggatgaggcacaaggccccattcccagccttgccagactacttgagccaattgtagttttatgcccttcccaagagctttctagggtttttgcttggatttgttttgttttgttaatactaggagtcattttcagtcctgggatacattgtgggataaatgagataactgagtgtaccttgaaatgctaccatagggccattgcagaaatgctattattattgtaaaatttagggtagaggggatttctgtggtttgttttctcatcatttaccccaacaaagagcttgttttgggaagttctttttttctgtccctttgagaaggtggcatgtgcataggcactgcaactaaccagaagtgggctgtctttgttggaagtgggggagatcacagagcagagtcagggtaaggggctcaagggccaccaggcagcactgccagagtgctggctggagcaggaagcctttcaggtaattagggaacccctttgtcaggctcagggtatctatttcctccaagctttccaaggcctgtccctatcctcactctaacaacacccccaggccctccccgtcacattcctcttcttcctttgggtccttcgtgcctgctgaccccacacccatatcctgtccagggctacatgtaagaggggaaaaataaaagggaaaagaattttatcagctattttccagaggggagaggactccaccctttgaatctctgccatatggcgacagaggaggagcaggcttggggacctctgactaattcagtggtccccagtgctccgcacttgtagacccaacccagttggcaccacctttgcagatgcatgtcttcagcagcatttctgttcggctgataaaacacacaggggcctggggcctctttttcttatccctttggctccacctcctgggcctcctcattcccttgtcctcatctccttccttcgcaatctttgtctcaaaaagcttagggcaagaagtgccaaactgcagcagataaactcagctgcagagtcctacgaaaacaaatgaaaatgaggaacaaattccacttcttaccgttggtctcattgcctgatttcctttagaccctgagctggagggcctgggtttactttcagggctggatggttacccaactccactctcaggtacttgggcctccctctctcattgactttggttgatctaactaaatggcttgcagtgtggagaattaaaaataagaaggtatggaaaaagaacacaacaccaccctccagaagaaaattctcacagtcgccagggctgtgagaattccatatttttgtgggcttggacttctctaatgagaacattctcttccctccttagctaataaatgaaaacatattatctatgacttttagtcccagagctggacgttgtgctaaggagaaagatgcgtgcatgttggaaagatggagagacaaacaaatagggatgcatataaaggtgtacttcttagtacctgtgctcttggagtccgtcggaatgagttacttcccttcagatgcaacggaaaaggcctttgggcttaattgggaagtaggcttcttctttatttggaaaaatgaaatcaaactgatacttcttagagaggagtggcatccctaggcagctgatttaagaaaatgtcttaagaggtctttgagtgacaaagcctagaaccccagacaatcctggatgagcaaaggtaggcaggaggctgaattgctgagcatgataaaatgaagtgaagccttgtgtttaggtgggcacaagagacacaaggctgataggactccagagcccaggaagagcatataaagggtggagaggtgcctggagtcatagcaggacagtggtgggtggaggatggaaaagacctgggaggaaatttttttttaatacatggcttcattagcacagtaatgtctaatatttcagggttatgccattgtctcccctgggctgccctgtaatctcgttccatgccctcaacctttcagtaaatactggctacacacactagccttgtgttagtggctctctggggagggtaaagtaacttactttaccttggctaacttacggtgaagtggtatactagtgcggacagggagccattgagccatggagccacaacccaggagtctgtggtgctggtaaaatggaggaagaggtgacagggagaaatgtaggtgcccagagaatcttcaaaataattagggttccacagtaagtggatggggtccaagacatgctattttatttatttatttatttatttatttatttgagaagaagtttctctcttgttgctcaggctggagtacaatggtgtgatctcggatcaccgcaacctctgcctcccaggttcaagcagttctccttcctcagcctcctaagtagctgggattacaggcatgcgccaccatgcccagctaattttgtatttttagtagagacgggttttctctatattggtcaggctggtctcgaactcctgacttcagatgatccacccacatcggcctcccaaaatgttgggattacaggcgtgagccatggtgtctggccaagacatgctatttgaatataaaacagaaatctcggtacactgggaaccctcaggccacagacccttggtttatatatactcacaccttagcttagcctttgaagcttaagaaggcatctgctcagtcccttgttctagtctaggatgactgaaattcatggtaggcctcagagttggacagccccctaacacggatgcctcctgagtgtttgcaggaagcatggatggatcacttgtctcccaaagtggctcatttcattcttataaatagctacatcattttacactttttccttggattgcagtgaaattggccttcccataacttctaatcattagttctaattctattctttgggctggtacagatttaatctggtccttcatgacaatacctgaagatgtttctagtgggccccactagattacaaactccttgagggattgatttaaatgacaatgacactaacacctaatggaggaaagagatccagctctcattttgtactctccacagcactgagcgagatcctttgcacatcactggcactgcagaaaaatgagaatgaatacacagaatgaatgtatcccttatccaaaatgcttaggaccagaagtgtttcaaatttcagattttttcagattttggaatatctgtgtaaacataatgagatgtctcagcaataggacccaagtctaagcatgaatttgtgtttcctgtataccttatatacatagcctgaagataattttagacaatatttttaataaacttgtggatgaaacaaagtgttgactgtgttttgactgtgacctgtcacatgaggtcaggtgtggaatcttccacttgcggcatcatgttggcactcaaaaagtttcgtattttggagtattttagaatttctgattttcagattaggggtgctcaaccagtgtaaataacatgcatcttaaagaaagttatttatttttcctccccaaattcttcttttccatatcaggggtcagcaaactttttctataaagggccagagagtaaatattgtaggctttaggggccatatatggtttctgatggatggtctactttgtcttatcttacgacacttttagaaatgtaaaagccggccacgcacggtggctcatgcctgcaatcccagcacttcgggaggccgaggcaggcagatcacctgaggtcaggagtttgagaccagcttggccaatatggctaaaccccgtctctactaaaaatacaaaaattagccgggcatggtggcacatgcctgtagtctcagctacttgggaggctgaggcaggagaatgtcttgaacctgggaggcagtggttgcagccagccaagatcgtgccactgcactccagcctgggcaacagagtgagactgtctcaagaaaaaaagaaagaaaggaatgtaaaagccattctttactgactgaccatacaaacacaacccctgagtcaggttaggttcactggccatagtttgccaaccccccatctagatgaaataacactagaccctcaacaatatgacttggtttctagaactctcctaatcctggttatcttcacatgaacactccccaatttaattaattcccccttaaaatataacatgagaacaagactgccatgactagaggtagctggcaggaacacacctgccacagttgtcctccaatcctattgcttttcctaaactccaaggattgtgctaagcaccaggagtaaaacacataagaataagaatggctccagcactttagaagtttatattcttactgaggtgacagttagacctatcctgaggaagcagcatatatgattttgtggcagatgagtcacagaaataagtatctcatgcccactgagaagcagaaggaaggggcctaaaacaaaagatatccactatttcttgaacttttaccagacaggataactgcttctacttcttagctcctttgttcctcaaaacagctaagacatagggactacttttatctctgttttacaggtgaggtttacagagttgaagtaacttcccaaaccacagctaaaacatggtaaagaagcctttgaatgtggcctgtctggctgcagagcctgagctctttgtctctgggctataccgccttcctggtgatacagtctgtggctcccaacggcctacttcctggcagaggctggcatgtcccttcatcctagttctgtaactctatggactaagattcccaggaactccttcgagatactcctcttgatccaggtgctagaatatgaaagcatgggcaaaataagactggcaggctaggagatcaggaaacagcaccaggaaaaaactaaggagggctgtgaaaagcaagaccaagtataggaaaactcagtctcttaaaatatggggatttagttattttggtagcagagctttctggatcgctgaggatttgcaaactcactttaaagaacaaaagagtcttcaatttactagtggcaggtagaaatattttcaaaatgtattgcacatgtctttgctttcttagaatacccccccgaaaaggactttatgactcagatgtatcatgaacatgtcgtggctatgttgcaactcagtgacatcattaatgcttattaaggtatagatatctaacctattcactcctaccataaatgattcaagaaaactggtgtgagttttcatacttatgtctgcttttcatttatttctgtttcttagttttcagctgttgcttctccttattatttgcttctaataggctcttcctacgtttcaactttagctaggatatcagataaccagggttattagaattatgtgtaaaacaagaataattgactctgaagaagtacctgaaagttcagtgcatgacttttactacatgaattttgtgacatctcttagaatcctcattttactaatttcttgagaccttgtctcctcccttgctcccagttcataagttgagatcaactagagtaactaagggtcaaatggagtccctagggaaacacagttaataaggatggactgagaagtaaacaacagaagagagctttttattcaactttaaattgtactataattcttgtagccttgtgggtagggaccgttcctcccatctagaatgaaaaagagtggaggtttcatcaattgtctcttaggatgtgacaagctctcactgactggtaggctcctgctactcccctacagagttgggagtcagaaggtcagataaagaaaggtcagtgtttgatggtttaatgccatggatgtgtgtgggaagacaggataaaaagagatctgtggtcagtcttgactctcataaatgtcattcctccagggctctagagactgtgaacctaaaagatgcactctccctgctgtgccgcagtgtttatgccaggattggctccaggcccctggctaaggtgtccccttgctctgcaccagctcaggcatttccagtcactcagtttggtctttctgccctcccacttgtcctgtccccctggtgtctcagagggtgctctgctcaccctggggtcagagccctgactctgaagttggggtccctttctcatttgcaggtggatcctgctactcattccacagcgctcggcctggtggtgggtggaggggctgctgcagctgcgagcctggccccagtgggtcagtagctggcttgggtccagagttgctttgctcttctggaacaggcacctctgtcctcagaactagcagcaggtctcactccacatctggaaaagttcaaaagtgtttgtctaggtgaaggaggcccttcgggggaggggattcaaaagcatgacatcatctcagtcaggcccagcttcctctggagagagaaaagggctgggccagggtggaccagatggggtccccagtaatcagtatgctgggggcgaagaggaagagagcaggcaagaagaggagtcttggaatggtgtcttgagaaggtgatcctttaattcagtgtctgaatactgcccccttggatgaattcagatgaatctgaccttaaagtcagagtggtgagagggtcccatctaaacaggaaagaagcctggattggcttctgcatcaatttattttgatcaacccacctcctgtttaagccagatgggagcccatccttttctctacagagatgtggggaataaattccgattttttggcagtttagacccagcctaacctccttgttgcatctactcaagagtcagctaaacgtcagtgttccattgctgcttcccttcatgaactatggccttccttttgggcagcaccttggtgattcaggctcttcatctctaagacagagatagtgatattccttctccccactgcttacctgacacataacagtaagacacgattgctgcagtcaccattaatagcaatctgcttgcagcaggttgtggaaggaagcagatatgagagagtgaaacccttgtcatcacctttagggaaaccgttcttggccccaggttgatggccgtgaagccaaaagtctaacttccctcttgatcctgagggaaacaagagatgtcattctaccttgctgtttacctggaaatcgctgaaaagtacagatctagatcccaatcaggaaaatttagagacaagctttagggtggcctgggaccctcagaggctcttccttgaagatgcaactgcctttatgaactctggataccaagttcacccttaacatttacaaggcccaaggcaagagtacaacagaggcctacatactatatttctaaacatttcaaggttgtaagtcaggccaactaactaataaaatatcttctatcctcttaccttgacaaacatatcttcgtgatcacctggaaggccaagtttcaatttgggatgctcagattcttggaattcactgccacagtgcagcttagcaggaagagatggctcctggccccagcccacttcccttcatctactttctacccatgactctggctcacattgcaaggcaattgcatgtgtgcctgggacaccccacccacaggcttaagcactaagcactaactcttctccctgcaagcagtcatcatttggccaccactgggccttgggggtgtacacccttgtgctgcagttccccttagcaggatggactgggagaagcggcccatcgtggatgtgagcaaagtgccatttgatcaagaagtgctaggacttctggtccccacagtatgacctagaagagagggtacatcctgattggtcctcacaggaagcaggacagggtgtgggctctttgaggcatagggttcagtcaagaagcttctccttcctgtgtctaatgaggatactgactgggaatggtgagtaacatctgtaatcccagagctttgggaggccaagacaggaggatcacttgaggccaggagcttaaggttacagtgagctatgatctcaccactgcattctagcctgggtgctgtgatgacagagcaagaccctgtcttaaaaaaaaaaaaaaaaaagtatgtatcccaggatatacagaagagatgtgcaaagacactcatgaagatcttcagagaaaaatgagatccaaaacattatactataatactataataacttagcatttgttatctcattgtttatcccagcaatccatgtaaggtagccagaaaagacccttacttaagaggtaagagagctgagatacatacatgcattaaattacatggttagttattggaagagccagaacaaagcttgtctaccttcccactacattgatgagaaaccagaccttgtgttccctggttcattagctgaattcctaggtcagagcatgctaaaagaagaaaaggaaaggcgaatgggtttgaaggccatgagcaaaaggatgaggggagtatgggaatgggaagtctgagaagtatagatttttaaggggatcgcatagagacagtgagtctagtgattattccaagagttagtatactggatggagagtgaccaaaaaagaggatacagtgaaaagaaaaaagaagcatgaccttttgcacacattagatctttgcaatatttatgcaggtgtaattttatattttagtatgtaactaaacatactatgaacatcacctcatagttccacgttgctacagtttttataatttttttctttctaatgtttgtttatatgattatataatgattatggtatattaagtgcatatatcacttatcctatcataattcatttagttgttcacgtttccctcctataaataatactgtatgagacatttgtatgcatataaatctttccttccctatgtcctcagggcaaatttggtagtgggaaattaatccactgtccaggagtatgttcttgttgctgccaacaaggggcagcagggaatgttctagacgaattctacctgaagtcaggaaattgcatcaggtgatatcaccaggctgatatctttaggttctgattcttcctcctcgtttccctatagcttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacaggtaaaaacagcatttgtgtcagatcccggagagatgctggtgatgcttgggtaaagcatttaggatgttttcagaccgctcccctctccacagaggaaattatacaagtccctagtattaatgacttgagtatcatgcttagggtgccctgaagtaagttctagataattcttccttcagtgacagttttctcaggcccatgtcttgggagctgatcctgatcagtaatgcctccataccccttctctcatgctgagtctagcttagtgataaaataaggatcagaagactcaatagagatctccgatctcttctaaaggaaagaaggtgggtacaggtccaaactgggggtctttggcttctctaaggtagaccagcatctatttcagtttcaggacacctctaatacattctagattctagctcttgttccaacagcttgaaatgagtttggtcagggatgggacacgaagtaactgttaactccccaaactcccttttctgtgttgaagtgcatgcccatatcatgacctgagattttgtgtatctatgagcacatgcacacatgcacacccatgttttggtctcttccagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggtgggtgaattgggccaaaaatggcagattgcccctcacttcatatgtatgcaggcaagctgtttctttccctccacccctctcctcatccctgccagtgggatttgggtcatgtgggaatcagcacggaaatacacagtttaaatattgctgggagaagtagaaagaggaggaaaggggtagagttaggtggtaaggcccatccaggctttgggtattgcattttagggaatagagagtaaacgggattctcagagatccatccaatcctctggttctttctagtacctatcagtggggctttgaccaggacaccatttccctttgggaatatttggcaactattgcttatttgctggggctgcttctcccacaatggttaggacaaataacatttcctctacatgagagggtttggtggttgtgccaggcacagagaggcagtaaaggggggcattggcaatggcaggaactgggcagaccagggatggtgagctcagcctggccttctcagccttagggcctatgactgtcaacagctttcagcatgcaggacagaaaatatgagggcctcagtcatgagatatgcccgagggatctttctgctttctgtttttaaaggagccagggccaggcgtggtggctcatgcctgtaatcccaacactttgggagcctgaagtgggcagatcacttgaggtcaggagttcaagaccagcccgggcaacatggtgaaaccccgtctctaccaaaacatacaaaaattagctgggtgtggtcatgcacctgtagtcccagctacccaggaggctgaggcaagagaatcacttgaaccagggaggtggaggttgcagtgagccaagatcatgccactgaactctaacctgggcaagactcgatctcaaaataaataaataaataaataaataaataaataaataaaagagccaaaatgggaatttggagagtcctggaggccaggagaaaaaacaggtaccccagcatctgctggtccaccccattatccctgtttgcctatatggccttcctgcaagtttgtgattgactggagagaaaacaacccaaatgggaaaagatcctcccctctcccagtcctgcaccatccaagccagtgtttaccaaggttgcaaatagctgcttgccagtaagcccaggctagtaacaaaagttttgtcatctgatgaggatagatatgaaatagacgagagtatgactattagaaatctctagtcccctgacaaattttaaatgtccttttccttaaaacctcctaagagagcacctcacagaaagcaaactggaaaaagttgggaaagaaatgaggagcaaagatactggcttatttactttgtttttgttattgtttttgttttaatacaggcagaaatcaaatgttcaggcatcagagaagtgaaattgactcagggcatctgcctggagcaaaataagacctccagctgtgtaagtcaaccccccacccaacctcttcctcccgcccctgtccctttcctccatcccttctgaacacccttaaaccttcttggattgcactggatttgagtaggggtccggggagtttagcttggtcagcctgcctttaatactgagctttctgtttagggagtaagaggccccatctggtgggcaaaaaagacaataacaattttaattattaataaattttgttatttttccatcctctatagtactttttccccctcctctatagtactttttaaattttcccctcctctgtagtacttttaaatctgagagagtcagatttaagttgctttttggttaagaaagtccttctcactcaccagatataaactagtcacctaaaatgtcagttcatttattatttcatttattattattaaatatttacacgtaactctttaatccatatagaatttattttgctgtaatgttatattttccatgtaaccgtttttcctaacattatttgttgaatgactccctcttctgttgcatgggatgtatcctttagcttatattaagtttttatatgtcttagggcatactcttcagacatattttatttgttcagtttgtctgtacttttgcctattttaaattttcaaaattattgtatgtttttactacagtatttgtaatatccagtcaggcagatctttttttattacactgtaaaaaatgtaaattatttttttctttttgaaaaaaatgcaaaaaaaatgctttaaaatttgaattgtaattgctctaaaatttcttcattttttctggttttcttttgcatctctcagcatcttgtttctttagcttttttctctagatttgatggatgtcataggcctatttgtcacatagatctcccactatatattcaaattgattgtcactgatagattaaaaagttaatagagttttgaacaattattttgggtttggccagtttcttgaatttgcctattgattctaggagctttttagttgattcctttcaattttaaggtaagctatcttgttatctactacaatatattgcttttctcctcctgtttagtagttatgcctcttgtttcagtttcatgtcttactgcattggaaagaatgtacagggaattgtgttattttaaatgattctggcagaccttgctttgtttcagtaaatctaatttcctaacagtaacatggctctggaggaaggatgaggtgaaggaaagagtggtatttacttacagaagaaaacattttcttacaagttggggatccatctaagggaatcacatatgtgatgtgctggcatgcatagactggaaagatcgcagcctgtagagggcttaaattacttattttttccattctaactcggtagttttactacgatcatcagttgggtctctctgacttacaaatttgtaaaaattatacctgaaagaatttaacttaggacatctaaaaacaacagaaatagtgaagacatgagtttgcatagtagaataactaaatgcagtctgcaaaatagttaacttaaacatgaacatacaatgtacaagtaacatgcagcatgtttctgatgctgattttcaactcaactcattagaaaaaaaattgtcaagttctttgctagttgtagccaaggaaaatgacgatctttccccctgcaaccccatcgacttggtggagcatacattttgctgtgtatcacacaggtatgtgccagaatgctaattctttgtgcctggaatttagagttaactgcagagcctttgcctttctgttgctcttcaaagctcacatgtaacttgggtcactgtatgagaaaagtgccagaaacttactgctcttttagaaataattcctcctaactctgctgtggtctttgctgtctggcctacttcgtgccacagctgagaaggtctctgcatgttcctctaagattgaggtagtatctgtgagtggggatgtgaaaagagagcagctcccctccagttcctatgccaaagaagacttccatccacactgccccttcataaagcaactctgtggcagctcatatgagtgcttttgttcccctggtctctccaccagtgttgatcccctttcttcctgagggctcctcatcctcagcccccatgcggcaaagcctcctgctactccccaaggtaatttatcctcagcttgctatcctgttccaaaatgtttagccacagctcccgattatgtccaagatgacctttccttggtccctactccctgcttgtctgataaacaatctgttccaaaagctgcgaatgctgcagagcctcctaattttccttaggttggatttgtactttcggtgtcagagggccttaaggtattggttctaatgttgggagcctcaggccctacctaataccctggtgaagagcaggaaggatttggtttccccgagcatcacatccttgatgtatgttggtatgctcctcatctaatatatgcccataacaggaatatatgtgcatgagtctagctatccagacactcattctgcactgacacatagtgagtaccaatatgttagctcaccctatgtggattctgtaggcgtgtcttctgtatgtgcattctgtagacatgtctatcccaatcactcttttttttttgctccccagccatcatctctaggcagccaggggaagggaaggtgataccactctcatctcaaacaccttttaccatatctcagcaccctcattagtaggatgtgtcttcctgagtctaactgtcattcttttggcagcagtgtcagcttaatttagtttttgtggttgtggttccccaaatagtttccaaaatgtaagattactttatactattatgctacacccatttgtccagagaaacccaccaaaatatttttaattgtggtaaaatatacataacataagtcatcaccttaaccacttttaagggtaccattgagtagtagtaagtacgttcacattattgtacaaccaatctctagaactgtctgcatcttccatcactgaaactgtgtacccatgaaacaactccctgctctccccagaccctggcaactttctgttcctatgttcctatgactttgcctactctaggtacctcatatgagtagagtcatagtgtttgtcttgttatgtgactggttgatttcactcagtataatgtctttaaagtccaaccatgttgtagcacgtgtcagaatttccttcctttttaaggatgaatgatattccattgtaagtgtaaaccttgttttgcttctccattcatctgttgatggacacttgggctttttcccctttaggctaatgtgaataatactgcttttaacataagtgtacaaatatctccttggcaacctgatttgagtccttttggggatatatccaaaagtgaaatggctggaccacatgggaattctatttttgaatttttgaggaaccaccagaccgttttacattcccaccagtagggtacaagggttccaatttcttctcatcctcatcaacacgtgttattttctgggtttttaaaatagcagccatcctaatgggtatgcggtggtatttcatggtggttttgatttgcatttccctgatgattagtgatgttgagcctctttcatgtgtttgttggccatttgtatatcttctttggggaaaagtctagtcaagtcctttgtccgccaatttgtatacctatctttagtaccacatctgtttatatgcagagtttgtaattactcagctagctcactttactcttattcacattgacttcatctgaggcaggctgcctccagcttgcactaaaggtcagtttccatgcggaaaacataagttacagatgaaggatttatgaagatggtgtgtctactggaaaaagcctgcaatctggaaacgatctcacttctcatccttcgttagctgcccccacttgttaggtgacttgaaacaggtcccttcattattccattttctatgtgaaaatagcgttttcacctgcattgtcaaactcacaccaaaatagataaaattatattaaaaggggttgggggagatgattgatcctagaatctcagcgttctaggatcttaatatcttaaatatcttaaaggtcattagaaaaatacctgtccctccctcacccccatctccgtccccattgcaatgcttgatgttaagagtctttgcaaagtttgagaatgctactctagagaggttagatgggtttattgtgtttgcagagaaacatgtgtgagctgatgtagttaggagcttgtaaatctgattctcatgaacaaaatggtgaggctgtgtcttcctcctgcccactctcccgtgatgtttgcccacagcctctcttgggggaggatgtttattccaagtgtccggagaagcattgtgctttgttcaggatccacctgctcctcttggggcccaaacacaattcactgatgcggttggattccagagtgggaggctggccgctattattcccttgaatctagtccaagaaactgtgcaaggggtggagggaccctgtgtggaggccaactggttgggatgttaagttgaacaacaattccctgagttcttttgtttggtgccacggttcatgcagtctgggttgtaaatgctataatgtgcactccccccaacacacacttaggagtcttacaatctagtcacagaaaaatgacaataacaatgcatagaatatgatcaatatgtagactctagatgatatggtatataaggtattactattattaataatgatagtagcagctgctatttatctagtgcccactagattctcaacaccacattggacacttttttttttttgagacagagtcttgctctgttgcccaggccggagtgcagagtgtagtgttgtgatctcagttcactgcaacctctgtctcctgggttcaagcgattctcgtgcctcagcctgccgagtagctgggattacagatgcccaccaccatgcccagctaatttttgcattttttagaagagacaaagtttcaccattttggccaggctggtctcgaacccttggcctcatctgatttgtccatctcggcctcccaaagtgctgggattacaggcatgagccactgcacccaactggacactttcatacaatatctatttagtcaccacaacaacctagtgtacagctgaggaacatgacactcagactagttgcatgacttgcaaagaatccagtaaagagcagagtaaggattgatgcccagtctggttgaaggtaggaccagcttcattgaggaggtagaaactgagttgagtcttgaaggatagtgaataccagggagaaggtagtgttcttcattctctttgaaatgagggttgtggaataatgcatgctgtccccatcttgggcttcctggaggtggggcctcagttccaaatgctcttccccactggcttcctctgctcaccctccagccattcttcaaggcctatagtgacatgtgtctgctaaccttaaccagacgtgtcctcacgctccttagtatgtccatagcactttggttgtacctttcttatggtgcttaccatggcagtagctctggcctctggagtctcttcacgcctccatttcttcatctacaaaattagtagaataactctaccaatctcgtaggtcgtgtgaggcctaaatgagttaatatatggaaagcactcagaactgtgcctggatatcataggcatctgttactttattttttatttctttatttttgagccagggtctctgtcacccagggtggagtacagtggactcaacctctcagggtggagagaccctcccacctcagccccccaagtagctggtactataagtgcatgccaccacacccggttgaatttctaaattttttatagagatagggtttccctgtgttgcccaggctggtctcgaattcctgggctgaagtaatcctcccacctcagcctcccaaagtgctaggatgacaggcatgagtcaccgcgcctggttggcatccatcattttaattgccgtacatgcaaactgaaagctttgacaaggtggaattcatagatacctcccaatccccacccccctacacatgacacatgaatacaacacacatacacatgacacttgcacagaggctttcacgcagtgtcctatattcagggcttgatacattgtcaaggtgtggcttcttgcactcagcaggtggaggaaggtgccatttggtttggctagtttacaacctgcaggagccctgtccctctgcctctcttgggaagcagacctatttcaactggagatacttcagaagcccagagtcatccaggcaagaaaccccctcctcctcatccagactctcggtgctggggagcgggggtgctcaaggggaagccatgtaaggctcccctgagacaactgggtttagagaggtggagactgttgattggttcagtgtggcattcagactacttagttcaaatgctgttcagaaaaacggatttttccagagttagaacgtctatccaaggacttactgggagacctgcagaattgctccttttcctgaggaatgaagcagcagtggcctgagaactcatttctctgtagccttgtttcctgggggttttttgaggctccagtttgggctcgtgtctctgtgacctggagtttggctaaccacactctcctggccttatccaagcccagttgttttccctcagctgcttcaaattccagctgggtcctgaggccaatcttgaccttgctttgtgtaggagcaaaggagcctgggttttcctgccttgggtcacagcagtgggaaaatacccaggctccattccaactgggaggaccctgtggccttgttgcaagcagcggccctgcccgcaaacaggaagctttctcctccacagagacccagttctgatgatggtcacacaccccagcagttttcccctaacaggaaagttgtcagggctgttcaggcatttccttctctgccatctgccatccggactgaagagaaagtcttagtttcaattcctttcctgttcaggggaggaggacactctgattggaggctgctggaataaattctgactcactattgaagaattatcagagtttttcttctgagtccaaattcctgggtctgatggcccagtcagcaacctgaagaaaaatctatgaacttcattaaaactgggttccatgcttttaatggaaaacagagatggaagacgagacaaaaatgccatttcagtttgagcaatgcactttttgagactcctttgggtagaaagagggaggaggccactgaggtttccactgagtgtatattatagatttgttctctcctcctcacctcccttggcttttcttccatcattggggactgctgtaatcttgaaacagaatattttctcttagttattattgcattattaatattttctgtttttggccatttcttttgagtttaatttgcatatgtgtacatgggcaatgcttcaacgagacaatttttttaagtttgttctctaacatcttagatactagaagatatggattttggtcctgggagattatttaggtttttttcttattgtgttgttgcacatatacagttagcagttttttttttttctgttggaattttagaaacaaggatccttcttgcggacggggtaagagtatgactgtaatcagagactctgggtgcagtttagggcagagcagtaatttcatctcttgctctccatgtttctctgtcttcccaggcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacaggtaaggtgcacctctgcctgggggaacagggaaggagtagggctgaggttagagaatcctgggtggagatggggcatcttagatccagagagaccacaggtgctggggagaaggacttggctggctttgggagcggtccccccgagatggaccaccctggccatcagagcatcttctagaacagcatgggagggtggagcaggataagctggtttctctttagatttagcaacccttgtttctaattctagaaatttccagcaaactccaacttatgaaaaagcaccaatctgacctgaaaaaggtaagtcctctgagatgatgggcctgagagggaatgccggggcaggggggctccctggagtaaggggggaagtcattatcttccccagttctacctaccatccttctctgagtcttctcctttctagtcttgaaccaaaaatggtggggtatgactgaattaagagacgtcagtctcaaatcccatttcaaaaattcctgaggtcaatgccctcaagcatctgttcactgttgagtcacacacagagggctggagcgggctgggctccacttttggcaagtgacaggctgtatcttctttgtcttcttttcaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctggtcagttctgggggccagggtaaaggaaatgaggaagatagtgggtttctggggagttcagtggatgtcatggagcaggaggagaaatactagaaaaagcccttctgtgagcttacataaagatatgcatgtgtgcacacacagtaatcggtggaagattcaaaatattatgtagcaaactggggaggggacagtaatggtgccagcccacctactcggtgtggtagcagatgattcgtgtcatctattcattatgttttctgggatagttcagcttggggccaccagaacaggctgtttaatcagccacactgtatttgcaaccgtgttaaatgcccagtgggtgcccccttgctcaaaaggaggtatacggaaggagaatccccatttgccattctggatgagggaggacaaggctgaggtctgaatcttggcctctggcctgtcccctaccctggggaggtcatcccacccttcttggaactgcccgttttcctgcgggagctggctgccgagctgcggtggcatggtgtggtgcctctcctatgtcctttctcctctaggagctggaaccctgaccactcttcaggaagaaaggagtctgcacatgcagctgcaccctccctccgatccttcctcccacctccccctcccccttctcccacccctgcccccacttcctgtttgggcccctctcccatccagtgtctcacagccctgcttaccagataatgctactttatttatacactgtctagggcgaagacccttattacacggaaaacggtggaggccagggctatagctcaggacctgggacctcccctgaggctcagggaaaggccagtgtgaaccgaggggctcaggaaaacgggaccggccaggccacctccagaaacggccattcagcaagacaacacgtggtggctgataccgaattgtgactcggctaggtggggcaaggctgggcagtgtccgagagagcacccctctctgcatctgaccacgtgctacccccatgctggaggtgacatctcttacgcccaacccttccccactgcacacacctcagaggctgttcttggggccctacaccttgaggaggggcaggtaaactcctgtcctttacacattcggctccctggagccagactctggtcttctttgggtaaacgtgtgacgggggaaagccaaggtctggagaagctcccaggaacaatcgatggccttgcagcactcacacaggacccccttcccctaccccctcctctctgccgcaatacaggaacccccaggggaaagatgagcttttctaggctacaattttctcccaggaagctttgatttttaccgtttcttccctgtattttctttctctactttgaggaaaccaaagtaaccttttgcacctgctctcttgtaatgatatagccagaaaaacgtgttgccttgaaccacttccctcatctctcctccaagacactgtggacttggtcaccagctcctcccttgttctctaagttccactgagctccatgtgccccctctaccatttgcagagtcctgcacagttttctggctggagcctagaacaggcctcccaagttttaggacaaacagctcagttctagtctctctggggccacacagaaactctttttgggctcctttttctccctctggatcaaagtaggcaggaccatgggaccaggtcttggagctgagcctctcacctgtactcttccgaaaaatcctcttcctctgaggctggatcctagccttatcctctgatctccatggcttcctcctccctcctgccgactcctgggttgagctgttgcctcagtcccccaacagatgcttttctgtctctgcctccctcaccctgagccccttccttgctctgcacccccatatggtcatagcccagatcagctcctaacccttatcaccagctgcctcttctgtgggtgacccaggtccttgtttgctgttgatttctttccagaggggttgagcagggatcctggtttcaatgacggttggaaatagaaatttccagagaagagagtattgggtagatattttttctgaatacaaagtgatgtgtttaaatactgcaattaaagtgatactgaaacacatctgttatgtgactctgtcttagctgggtgtgtctgcatgcaagagtgacaccctccattagacctagctagactgtgcagtgatgtggtggggaggaccagccagggaagagggagcacctcagcagacacaggcaccagccaggatgctaaggacctttagccaagtctgccaactattctcctccatggggagaggaaacatccatttccagtggtagaaaggcagacccgaatgtaccagggagcttccaaatggagggtggtatgttgggttcttaggagctgtacccttcatgaacacccttctgagaagaggagcatgctgatcactgctgcaaaatatgcaaaacaaagggaaggggcaatgtcctgtgcaccctttattatcaggccacccccctccccagccccccaggtcagagtagacacagtgaaggactatgtggggactgttgttctagagacctggcagccaactcagggagggggctggtttccaccctcaagattaagacagcagcctaattaaaaaaaaaatctgtaagcatgtacctccccccagcttccaaaacaacccccaccccacccctaccaggccataggaagttggggagggagtgctgaggagctccaggaaacactcccaagtgtgtcgacagtggcagaggcagttggggccaaacaaaggttgattcttccattcttatctccataaagccagacctttcccttcagcactcctccacccccatctccttcttgcttttctccaactcctctaatcataggttcttccctaggacagaggggaggcgaaatgatgaggttcagagtcttccctcaaaggcgatggctgccttgagggttggagcaaaggatgatgagcaaaagacgatggtaatcagtagggaagtccagcccacttgcatctagttgcacatcttgccttgagagtaatccagtgagggtctgtcccagctaggacatcaagtaggaggggtgggttcagggttcagattcctaggaaatatgggaggagaggaaaaggcaacttggatgcacctccagcttcaggcctagcaacctgcaatgcatctcaccctgagtttgctggaatgtgtatgtatgctttgggaggaagggctgtgtgtgtattgcggggtggggtggggcagctggttccctctgacagctggacagcttgccctgaagaatttgcctgctttctggaaaaatccaactttcccaccgtgggcctgagcgtcctggtacagcaatggcgccacctgctggccttattgaggtcctactgctcagcctcagctcaatcgcctccatgttgggcttctctccctggctgccccaccctctagtccaatttctcttgtacacaaagctcatataactatagaacgtcactgttgaagagaactttaaagatacatttaattaaactcccttatggtatagttaaagacaaactaaggctcagagaagggaggtggcttgcccaatcacccagaattccaaagtcctgaatctgtagttttcccttccatcatatcatcctactcttctgccgagtcctccgtgttactccagttggatgtcatgaagccagtgtggcagtgtgaagataggtttgggacttcacttctggagcatttcatcaacataagctatcctaggcctggccagccaagcaggtcctggaggagccccaggacaaagatcacaggaggccatgaggttcggcttcttcggcgcccacagtgagcccaggaaaattagctgtagggtattacactgttgactatggagagcatatctggaattatcttcagccagattttcatctgaatggataaatgggaataccatctaagtccagataaatagatcacttccatctcatcccttctaggtagattaatcccacacttcctcttcacacaaaaccagtaataggtcatcgattttgtgcaacaggatgctgcttctcttcctaaagcccccatcgaagaggcttccagccaccattcaatcattcatcaagtcttatgatgtgccagacactgcgcgaaatgtgccagaacatctgttatgtgccagacactgttcttgagactggggatacagcaaacactcatgaagcttataattctagcagaagaggacagtaaacaatgtcatctcagtaagtatatacatgtgttttcaggattgagagctatgaaaaacataaaatatattgagaataatggttggtattttacatatggtggttacttttagaaaaataacagtggagagcacagcttcacttgaatgaagtggagaagcaggttgtatgccaagctgggagagattatcccacacaggggaaaggacaagtgcaaagccctatgatgaaaagctgccaagtgcagaaagcctcagatggcagggggcaagatggccatgaggttgtgtcagtgagtgggggtggggagaggcaggaggtcagactacatggggcctttttagttgtagattgggaagccactggagggttttgagcagagaagtcatatcatctgctttatgttttaaaaggatcatgctggctgctgagtagagaatagaggttgagggataagaaagtagaaggagaccgtagcaagaagaacgatcatggctgggagcaggtgatcatattggcagtgatgagatcaagcagaattcaaaaagtggtttcaaagtagaggtaacaggacttgctcagtctatttatttcttcaaataataatcatatttacaatgatagtagctaacagtttttgagtgcttactgtatgaaaattgagatatggtgccaatatttaaatagcatattttacttaacattcacagaaaccctgtgaagtaggttctattatctcagaaaaagaaactgaaactcagagaataacaagggactgtgttacgtgcacagtggcagaggcaaagatgaataggatgtgagtttatttgaaccccaaatgtttaaatcttggggataatacaacacacatttaaacaaagaagcaagaaaaaaaatgcacaacagaaagtgagaaataacacgaggaaagactaaatgaagtgctttgtatctagatgtgggcaggaccctttccagctgagaagatctgagactgggtcatgaacaggtggtttctgagtgggtcctgtaaaaatgaatacgattttgatgatagtaatgagtaaggacatttgagactgatagaagagtacatacaatatgtagtgatggggaaagataaggtactgtcaaaggacaatgtgttttctggtatgacagagaagtagaatgtgttaagggaagccgagtaccagaaagatccgggtgtcacagtttgtgtagggtgtttaaagctaaaccacagagtttaattttatccaatagaagaggagccacagaagagtttccatttattcattaatttattcatttattcaaaaaatatttgagtgcttattataagccaggtactatgccaggcacctgggataagacatagtcccttctgtcaagtctttacattgggtggatgtgggagggacagatgacagaacaatatgcattgagtgtaagtgctatggtataggaagctctgagtgggaggggcatggaagccgtggaagaccatggaaggcttcccaggagaagtgacgtctggactgatcctttggtcaagcaggagttaaagaggagaaaaggagagatatgggtgttcccgagagaggaagaagccttgtcccaggagcaaagtgagggtgattgttccagaaatgtgagtgattcttttaaggctcaagcaaagcatgtgattcttctttataccttctatttctttgctgagtgtttctgttcttttgtttcaagcatgctgcaattgctcattaaagcatgtttatgatggctgtctgttttaaaattcttgtcagatggtttcaacatctttatcatctcaatgttggcatctgttaatggttttttctcaatcaaattgagattttcctggttcttggtattaccagtgattttaattgcatctggaaatttgggatttatgttgaaagactggatcttattgaaagattctgtttagcacccctcctttgataccacactggtgggtccaggttccccattcagctgttgacaccttcagggcagagaggtgggatggggtgaagggggtacctcattattgctggcccaggttagaagttcaggcttcccagtagatctctgctgataccaccctggtgccatgtcattccttgagtccaaaagtccctcccaattctgccttcttctctctacatatcggagtctccctatgtttgacttatatataatgtccagggtttttagagttagttaacaggaggcataagaaaaagtgtgtccactccatcttgtctggaactggaagttcaagtcgaatataagagagaggagaggaaattacaagccatgagactggagagttaggcaggttctacaccagctattctcaaagccctcttacactcttaaaaatttagaacttcaaagagcttttgattttgaaagttacatctatcaattattactgtttcaaaaattaaaattgagaaaattttatttattaatttgtttaaaaataacaataattattcaattacatgataatgtaagtaatgcttttcttaatgaaaaataattatattttccaaaacaaaaacaattaggaaaaagagtgtcattgttttagactttggtaaatctctctaatatctggctgaagagaagaatgctgattctttttttttttttttttttttgagacggagtctcgctctgtcacccaggctggagtgtagtggtgtgatctcggctcactgcaagctctgcctcccgggttcacgccattctcctgcctcagcctcccaagtagctgggactacaggcacccgccaccacgcccggctaatttttttgtatttttagtagagatggggtttcaccgtgttagccaggctggtctcgatctcctgacctcatgatccacccacctcagcctcccaaagcgctgggattacaggtgtgagacaccgcgcccagcccccgaatgctgattcttttatctgcttctgtattcaatctgttgtgatatgatgggtagcctctgaaacactccactgtatacttgtgaaagaatgaatgtgaaaaaggaaaatagatttgtagtattattattcaaattgttttgacctcagagaccacttggaaatgttttagggaacccccagaggaccttggatcatgctttgagaaccgcggctctagatatgttactatttcagtagcatctaagtacatgtggctgctgagcacttgtaatgtggctagtgcaaatgagagacaggacttccagctatatgtaatttaataaactcaaatttaaaaactggaacctcataaaatgttttgttgttgttgttaaacatgaccttatagttttggtaggaa

SEQ ID NO: 189 - DNA, 7969 bp, Homo sapiens
  source 1..7969: mol_type = genomic DNA
agtgtcttccactcggtgcgtctctctaggagccgcgcgggaaggatgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctgggcgaagacccttattacacggaaaacggtggaggccagggctatagctcaggacctgggacctcccctgaggctcagggaaaggccagtgtgaaccgaggggctcaggaaaacgggaccggccaggccacctccagaaacggccattcagcaagacaacacgtggtggctgataccgaattgtgactcggctaggtggggcaaggctgggcagtgtccgagagagcacccctctctgcatctgaccacgtgctacccccatgctggaggtgacatctcttacgcccaacccttccccactgcacacacctcagaggctgttcttggggccctacaccttgaggaggggcaggtaaactcctgtcctttacacattcggctccctggagccagactctggtcttctttgggtaaacgtgtgacgggggaaagccaaggtctggagaagctcccaggaacaatcgatggccttgcagcactcacacaggacccccttcccctaccccctcctctctgccgcaatacaggaacccccaggggaaagatgagcttttctaggctacaattttctcccaggaagctttgatttttaccgtttcttccctgtattttctttctctactttgaggaaaccaaagtaaccttttgcacctgctctcttgtaatgatatagccagaaaaacgtgttgccttgaaccacttccctcatctctcctccaagacactgtggacttggtcaccagctcctcccttgttctctaagttccactgagctccatgtgccccctctaccatttgcagagtcctgcacagttttctggctggagcctagaacaggcctcccaagttttaggacaaacagctcagttctagtctctctggggccacacagaaactctttttgggctcctttttctccctctggatcaaagtaggcaggaccatgggaccaggtcttggagctgagcctctcacctgtactcttccgaaaaatcctcttcctctgaggctggatcctagccttatcctctgatctccatggcttcctcctccctcctgccgactcctgggttgagctgttgcctcagtcccccaacagatgcttttctgtctctgcctccctcaccctgagccccttccttgctctgcacccccatatggtcatagcccagatcagctcctaacccttatcaccagctgcctcttctgtgggtgacccaggtccttgtttgctgttgatttctttccagaggggttgagcagggatcctggtttcaatgacggttggaaatagaaatttccagagaagagagtattgggtagatattttttctgaatacaaagtgatgtgtttaaatactgcaattaaagtgatactgaaacacatctgttatgtgactctgtcttagctgggtgtgtctgcatgcaagagtgacaccctccattagacctagctagactgtgcagtgatgtggtggggaggaccagccagggaagagggagcacctcagcagacacaggcaccagccaggatgctaaggacctttagccaagtctgccaactattctcctccatggggagaggaaacatccatttccagtggtagaaaggcagacccgaatgtaccagggagcttccaaatggagggtggtatgttgggttcttaggagctgtacccttcatgaacacccttctgagaagaggagcatgctgatcactgctgcaaaatatgcaaaacaaagggaaggggcaatgtcctgtgcaccctttattatcaggccacccccctccccagccccccaggtcagagtagacacagtgaaggactatgtggggactgttgttctagagacctggcagccaactcagggagggggctggtttccaccctcaagattaagacagcagcctaattaaaaaaaaaatctgtaagcatgtacctccccccagcttccaaaacaacccccaccccacccctaccaggccataggaagttggggagggagtgctgaggagctccaggaaacactcccaagtgtgtcgacagtggcagaggcagttggggccaaacaaaggttgattcttccattcttatctccataaagccagacctttcccttcagcactcctccacccccatctccttcttgcttttctccaactcctctaatcataggttcttccctaggacagaggggaggcgaaatgatgaggttcagagtcttccctcaaaggcgatggctgccttgagggttggagcaaaggatgatgagcaaaagacgatggtaatcagtagggaagtccagcccacttgcatctagttgcacatcttgccttgagagtaatccagtgagggtctgtcccagctaggacatcaagtaggaggggtgggttcagggttcagattcctaggaaatatgggaggagaggaaaaggcaacttggatgcacctccagcttcaggcctagcaacctgcaatgcatctcaccctgagtttgctggaatgtgtatgtatgctttgggaggaagggctgtgtgtgtattgcggggtggggtggggcagctggttccctctgacagctggacagcttgccctgaagaatttgcctgctttctggaaaaatccaactttcccaccgtgggcctgagcgtcctggtacagcaatggcgccacctgctggccttattgaggtcctactgctcagcctcagctcaatcgcctccatgttgggcttctctccctggctgccccaccctctagtccaatttctcttgtacacaaagctcatataactatagaacgtcactgttgaagagaactttaaagatacatttaattaaactcccttatggtatagttaaagacaaactaaggctcagagaagggaggtggcttgcccaatcacccagaattccaaagtcctgaatctgtagttttcccttccatcatatcatcctactcttctgccgagtcctccgtgttactccagttggatgtcatgaagccagtgtggcagtgtgaagataggtttgggacttcacttctggagcatttcatcaacataagctatcctaggcctggccagccaagcaggtcctggaggagccccaggacaaagatcacaggaggccatgaggttcggcttcttcggcgcccacagtgagcccaggaaaattagctgtagggtattacactgttgactatggagagcatatctggaattatcttcagccagattttcatctgaatggataaatgggaataccatctaagtccagataaatagatcacttccatctcatcccttctaggtagattaatcccacacttcctcttcacacaaaaccagtaataggtcatcgattttgtgcaacaggatgctgcttctcttcctaaagcccccatcgaagaggcttccagccaccattcaatcattcatcaagtcttatgatgtgccagacactgcgcgaaatgtgccagaacatctgttatgtgccagacactgttcttgagactggggatacagcaaacactcatgaagcttataattctagcagaagaggacagtaaacaatgtcatctcagtaagtatatacatgtgttttcaggattgagagctatgaaaaacataaaatatattgagaataatggttggtattttacatatggtggttacttttagaaaaataacagtggagagcacagcttcacttgaatgaagtggagaagcaggttgtatgccaagctgggagagattatcccacacaggggaaaggacaagtgcaaagccctatgatgaaaagctgccaagtgcagaaagcctcagatggcagggggcaagatggccatgaggttgtgtcagtgagtgggggtggggagaggcaggaggtcagactacatggggcctttttagttgtagattgggaagccactggagggttttgagcagagaagtcatatcatctgctttatgttttaaaaggatcatgctggctgctgagtagagaatagaggttgagggataagaaagtagaaggagaccgtagcaagaagaacgatcatggctgggagcaggtgatcatattggcagtgatgagatcaagcagaattcaaaaagtggtttcaaagtagaggtaacaggacttgctcagtctatttatttcttcaaataataatcatatttacaatgatagtagctaacagtttttgagtgcttactgtatgaaaattgagatatggtgccaatatttaaatagcatattttacttaacattcacagaaaccctgtgaagtaggttctattatctcagaaaaagaaactgaaactcagagaataacaagggactgtgttacgtgcacagtggcagaggcaaagatgaataggatgtgagtttatttgaaccccaaatgtttaaatcttggggataatacaacacacatttaaacaaagaagcaagaaaaaaaatgcacaacagaaagtgagaaataacacgaggaaagactaaatgaagtgctttgtatctagatgtgggcaggaccctttccagctgagaagatctgagactgggtcatgaacaggtggtttctgagtgggtcctgtaaaaatgaatacgattttgatgatagtaatgagtaaggacatttgagactgatagaagagtacatacaatatgtagtgatggggaaagataaggtactgtcaaaggacaatgtgttttctggtatgacagagaagtagaatgtgttaagggaagccgagtaccagaaagatccgggtgtcacagtttgtgtagggtgtttaaagctaaaccacagagtttaattttatccaatagaagaggagccacagaagagtttccatttattcattaatttattcatttattcaaaaaatatttgagtgcttattataagccaggtactatgccaggcacctgggataagacatagtcccttctgtcaagtctttacattgggtggatgtgggagggacagatgacagaacaatatgcattgagtgtaagtgctatggtataggaagctctgagtgggaggggcatggaagccgtggaagaccatggaaggcttcccaggagaagtgacgtctggactgatcctttggtcaagcaggagttaaagaggagaaaaggagagatatgggtgttcccgagagaggaagaagccttgtcccaggagcaaagtgagggtgattgttccagaaatgtgagtgattcttttaaggctcaagcaaagcatgtgattcttctttataccttctatttctttgctgagtgtttctgttcttttgtttcaagcatgctgcaattgctcattaaagcatgtttatgatggctgtctgttttaaaattcttgtcagatggtttcaacatctttatcatctcaatgttggcatctgttaatggttttttctcaatcaaattgagattttcctggttcttggtattaccagtgattttaattgcatctggaaatttgggatttatgttgaaagactggatcttattgaaagattctgtttagcacccctcctttgataccacactggtgggtccaggttccccattcagctgttgacaccttcagggcagagaggtgggatggggtgaagggggtacctcattattgctggcccaggttagaagttcaggcttcccagtagatctctgctgataccaccctggtgccatgtcattccttgagtccaaaagtccctcccaattctgccttcttctctctacatatcggagtctccctatgtttgacttatatataatgtccagggtttttagagttagttaacaggaggcataagaaaaagtgtgtccactccatcttgtctggaactggaagttcaagtcgaatataagagagaggagaggaaattacaagccatgagactggagagttaggcaggttctacaccagctattctcaaagccctcttacactcttaaaaatttagaacttcaaagagcttttgattttgaaagttacatctatcaattattactgtttcaaaaattaaaattgagaaaattttatttattaatttgtttaaaaataacaataattattcaattacatgataatgtaagtaatgcttttcttaatgaaaaataattatattttccaaaacaaaaacaattaggaaaaagagtgtcattgttttagactttggtaaatctctctaatatctggctgaagagaagaatgctgattctttttttttttttttttttttgagacggagtctcgctctgtcacccaggctggagtgtagtggtgtgatctcggctcactgcaagctctgcctcccgggttcacgccattctcctgcctcagcctcccaagtagctgggactacaggcacccgccaccacgcccggctaatttttttgtatttttagtagagatggggtttcaccgtgttagccaggctggtctcgatctcctgacctcatgatccacccacctcagcctcccaaagcgctgggattacaggtgtgagacaccgcgcccagcccccgaatgctgattcttttatctgcttctgtattcaatctgttgtgatatgatgggtagcctctgaaacactccactgtatacttgtgaaagaatgaatgtgaaaaaggaaaatagatttgtagtattattattcaaattgttttgacctcagagaccacttggaaatgttttagggaacccccagaggaccttggatcatgctttgagaaccgcggctctagatatgttactatttcagtagcatctaagtacatgtggctgctgagcacttgtaatgtggctagtgcaaatgagagacaggacttccagctatatgtaatttaataaactcaaatttaaaaactggaacctcataaaatgttttgttgttgttgttaaacatgaccttatagttttggtaggaa

SEQ ID NO: 190 - protein, 385 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLGEDPYYTENGGGQGYSSGPGTSPEAQGKASVNRGAQENGTGQATSRNGHSARQHVVADTEL

SEQ ID NO: 191 - DNA, 103 bp, Synthetic construct
  source 1..103: mol_type = other DNA
ctgggaccttaacagaaatgtgaccgatatcgagtgtgttaaagacgccgactattctatgccggtaaatcatactctctattgtttttttatttttatttta

SEQ ID NO: 192 - DNA, 18 bp, Synthetic construct
  source 1..18: mol_type = other DNA
gccaaagattgccctgag

SEQ ID NO: 193 - DNA, 18 bp, Synthetic construct
  source 1..18: mol_type = other DNA
gccaaaaattgccctgag

SEQ ID NO: 194 - DNA, 18 bp, Synthetic construct
  source 1..18: mol_type = other DNA
gccaaagattgttctgag

SEQ ID NO: 195 - DNA, 20 bp, Homo sapiens
  source 1..20: mol_type = genomic DNA
tcgagtgtgttaaagacgcc

SEQ ID NO: 196 - DNA, 20 bp, Homo sapiens
  source 1..20: mol_type = genomic DNA
atagaatagtcggcgtcttt

SEQ ID NO: 197 - protein, 6 aa, Synthetic construct
AKDCPE

SEQ ID NO: 198 - protein, 6 aa, Synthetic construct
AKNCPE

SEQ ID NO: 199 - protein, 6 aa, Synthetic construct
AKDCSE

SEQ ID NO: 200 - protein, 11 aa, Homo sapiens
TNYTVRVANPP

SEQ ID NO: 201 - protein, 11 aa, Pan troglodytes
TNYTVQVVNPP

SEQ ID NO: 202 - protein, 11 aa, Macaca mulatta
TNYTVRVASPP

SEQ ID NO: 203 - protein, 21 aa, Synthetic construct
WDLNRNVTDIECVKDADYSMP

SEQ ID NO: 204 - DNA, 20 bp, Homo sapiens
  source 1..20: mol_type = genomic DNA
ggcaaggctcacccgttcca

SEQ ID NO: 205 - RNA, 20 nt, Homo sapiens
  source 1..20: mol_type = genomic RNA
tggaacgggtgagccttgcc

SEQ ID NO: 206 - DNA, 20 bp, Homo sapiens
  source 1..20: mol_type = genomic DNA
gttccaactcgaagtgccag

SEQ ID NO: 207 - RNA, 20 nt, Homo sapiens
  source 1..20: mol_type = genomic RNA
ctggcacttcgagttggaac

SEQ ID NO: 208 - DNA, 20 bp, Homo sapiens
  source 1..20: mol_type = genomic DNA
ggcacttcgagttggaacgg

SEQ ID NO: 209 - RNA, 20 nt, Homo sapiens
  source 1..20: mol_type = genomic RNA
ccgttccaactcgaagtgcc

SEQ ID NO: 210 - DNA, 20 bp, Homo sapiens
  source 1..20: mol_type = genomic DNA
ttccaactcgaagtgccagg

SEQ ID NO: 211 - RNA, 20 nt, Homo sapiens
  source 1..20: mol_type = genomic RNA
cctggcacttcgagttggaa

SEQ ID NO: 212 - DNA, 20 bp, Homo sapiens
  source 1..20: mol_type = genomic DNA
tggcacttcgagttggaacg

SEQ ID NO: 213 - RNA, 20 nt, Homo sapiens
  source 1..20: mol_type = genomic RNA
cgttccaactcgaagtgcca

SEQ ID NO: 214 - DNA, 20 bp, Homo sapiens
  source 1..20: mol_type = genomic DNA
gctggccctggcacttcgag

SEQ ID NO: 215 - RNA, 20 nt, Homo sapiens
  source 1..20: mol_type = genomic RNA
ctcgaagtgccagggccagc

SEQ ID NO: 216 - DNA, 20 bp, Homo sapiens
  source 1..20: mol_type = genomic DNA
ccagctggccctggcacttc

SEQ ID NO: 217 - RNA, 20 nt, Homo sapiens
  source 1..20: mol_type = genomic RNA
gaagtgccagggccagctgg

SEQ ID NO: 218 - DNA, 20 bp, Homo sapiens
  source 1..20: mol_type = genomic DNA
gggccagctggaggtctacc

SEQ ID NO: 219 - RNA, 20 nt, Homo sapiens
  source 1..20: mol_type = genomic RNA
ggtagacctccagctggccc

SEQ ID NO: 220 - DNA, 20 bp, Homo sapiens
  source 1..20: mol_type = genomic DNA
tgaggtagacctccagctgg

SEQ ID NO: 221 - RNA, 20 nt, Homo sapiens
  source 1..20: mol_type = genomic RNA
ccagctggaggtctacctca

SEQ ID NO: 222 - DNA, 20 bp, Homo sapiens
  source 1..20: mol_type = genomic DNA
gctggaggtctacctcaagg

SEQ ID NO: 223 - RNA, 20 nt, Homo sapiens
  source 1..20: mol_type = genomic RNA
ccttgaggtagacctccagc

SEQ ID NO: 224 - DNA, 20 bp, Homo sapiens
  source 1..20: mol_type = genomic DNA
tccttgaggtagacctccag

SEQ ID NO: 225 - RNA, 20 nt, Homo sapiens
  source 1..20: mol_type = genomic RNA
ctggaggtctacctcaagga

SEQ ID NO: 226 - DNA, 20 bp, Homo sapiens
  source 1..20: mol_type = genomic DNA
tccgtccttgaggtagacct

SEQ ID NO: 227 - RNA, 20 nt, Homo sapiens
  source 1..20: mol_type = genomic RNA
aggtctacctcaaggacgga

SEQ ID NO: 228 - DNA, 20 bp, Homo sapiens
  source 1..20: mol_type = genomic DNA
caaggacggatggcacatgg

SEQ ID NO: 229 - RNA, 20 nt, Homo sapiens
  source 1..20: mol_type = genomic RNA
ccatgtgccatccgtccttg

SEQ ID NO: 230 - DNA, 20 bp, Homo sapiens
  source 1..20: mol_type = genomic DNA
tgtgccatccgtccttgagg

SEQ ID NO: 231 - RNA, 20 nt, Homo sapiens
  source 1..20: mol_type = genomic RNA
cctcaaggacggatggcaca

SEQ ID NO: 232 - DNA, 20 bp, Homo sapiens
  source 1..20: mol_type = genomic DNA
gctctggctgcaaaccatgt

SEQ ID NO: 233 - RNA, 20 nt, Homo sapiens
  source 1..20: mol_type = genomic RNA
acatggtttgcagccagagc

SEQ ID NO: 234 - DNA, 20 bp, Homo sapiens
  source 1..20: mol_type = genomic DNA
gccccagctctggctgcaaa

SEQ ID NO: 235 - RNA, 20 nt, Homo sapiens
  source 1..20: mol_type = genomic RNA
tttgcagccagagctggggc

SEQ ID NO: 236 - DNA, 20 bp, Homo sapiens
  source 1..20: mol_type = genomic DNA
gagctggggccggagctcca

SEQ ID NO: 237 - RNA, 20 nt, Homo sapiens
  source 1..20: mol_type = genomic RNA
tggagctccggccccagctc

SEQ ID NO: 238 - DNA, 20 bp, Homo sapiens
  source 1..20: mol_type = genomic DNA
actgcttggagctccggccc

SEQ ID NO: 239 - RNA, 20 nt, Homo sapiens
  source 1..20: mol_type = genomic RNA
gggccggagctccaagcagt

SEQ ID NO: 240 - DNA, 20 bp, Homo sapiens
  source 1..20: mol_type = genomic DNA
cccactgcttggagctccgg

SEQ ID NO: 241 - RNA, 20 nt, Homo sapiens
  source 1..20: mol_type = genomic RNA
ccggagctccaagcagtggg

SEQ ID NO: 242 - DNA, 20 bp, Homo sapiens
  source 1..20: mol_type = genomic DNA
gagctccaagcagtgggagg

SEQ ID NO: 243 - RNA, 20 nt, Homo sapiens
  source 1..20: mol_type = genomic RNA
cctcccactgcttggagctc

SEQ ID NO: 244 - DNA, 20 bp, Homo sapiens
  source 1..20: mol_type = genomic DNA
cagtcaagcgtcaaaagtct

SEQ ID NO: 245 - RNA, 20 nt, Homo sapiens
  source 1..20: mol_type = genomic RNA
agacttttgacgcttgactg

SEQ ID NO: 246 - DNA, 20 bp, Homo sapiens
  source 1..20: mol_type = genomic DNA
agtcaagcgtcaaaagtctg

SEQ ID NO: 247 - RNA, 20 nt, Homo sapiens
  source 1..20: mol_type = genomic RNA
cagacttttgacgcttgact

SEQ ID NO: 248 - DNA, 20 bp, Homo sapiens
  source 1..20: mol_type = genomic DNA
gtcaagcgtcaaaagtctgc

SEQ ID NO: 249 - RNA, 20 nt, Homo sapiens
  source 1..20: mol_type = genomic RNA
gcagacttttgacgcttgac

SEQ ID NO: 250 - DNA, 20 bp, Homo sapiens
  source 1..20: mol_type = genomic DNA
cagttcagccgctggcagac

SEQ ID NO: 251 - RNA, 20 nt, Homo sapiens
  source 1..20: mol_type = genomic RNA
gtctgccagcggctgaactg

SEQ ID NO: 252 - DNA, 20 bp, Homo sapiens
  source 1..20: mol_type = genomic DNA
acagttcagccgctggcaga

SEQ ID NO: 253 - RNA, 20 nt, Homo sapiens
  source 1..20: mol_type = genomic RNA
tctgccagcggctgaactgt

SEQ ID NO: 254 - DNA, 20 bp, Homo sapiens
  source 1..20: mol_type = genomic DNA
cacagttcagccgctggcag

SEQ ID NO: 255 - RNA, 20 nt, Homo sapiens
  source 1..20: mol_type = genomic RNA
ctgccagcggctgaactgtg

SEQ ID NO: 256 - DNA, 20 bp, Homo sapiens
  source 1..20: mol_type = genomic DNA
gcggctgaactgtggggtgc

SEQ ID NO: 257 - RNA, 20 nt, Homo sapiens
  source 1..20: mol_type = genomic RNA
gcaccccacagttcagccgc

SEQ ID NO: 258 - DNA, 20 bp, Homo sapiens
  source 1..20: mol_type = genomic DNA
ttaagccttggccccttcct

SEQ ID NO: 259 - RNA, 20 nt, Homo sapiens
  source 1..20: mol_type = genomic RNA
aggaaggggccaaggcttaa

SEQ ID NO: 260 - DNA, 20 bp, Homo sapiens
  source 1..20: mol_type = genomic DNA
taagccttggccccttcctt

SEQ ID NO: 261 - RNA, 20 nt, Homo sapiens
  source 1..20: mol_type = genomic RNA
aaggaaggggccaaggctta

SEQ ID NO: 262 - DNA, 20 bp, Homo sapiens
  source 1..20: mol_type = genomic DNA
tggccccttccttgtcacct

SEQ ID NO: 263 - RNA, 20 nt, Homo sapiens
  source 1..20: mol_type = genomic RNA
aggtgacaaggaaggggcca

SEQ ID NO: 264 - DNA, 20 bp, Homo sapiens
  source 1..20: mol_type = genomic DNA
cttccttgtcacctacacac

SEQ ID NO: 265 - RNA, 20 nt, Homo sapiens
  source 1..20: mol_type = genomic RNA
gtgtgtaggtgacaaggaag

SEQ ID NO: 266 - DNA, 20 bp, Homo sapiens
  source 1..20: mol_type = genomic DNA
ttccttgtcacctacacacc

SEQ ID NO: 267 - RNA, 20 nt, Homo sapiens
  source 1..20: mol_type = genomic RNA
ggtgtgtaggtgacaaggaa

SEQ ID NO: 268 - DNA, 20 bp, Homo sapiens
  source 1..20: mol_type = genomic DNA
acacacctcagagctcaatc

SEQ ID NO: 269 - RNA, 20 nt, Homo sapiens
  source 1..20: mol_type = genomic RNA
gattgagctctgaggtgtgt

SEQ ID NO: 270 - DNA, 20 bp, Homo sapiens
  source 1..20: mol_type = genomic DNA
cagagctcaatcatctgcta

SEQ ID NO: 271 - RNA, 20 nt, Homo sapiens
  source 1..20: mol_type = genomic RNA
tagcagatgattgagctctg

SEQ ID NO: 272 - DNA, 20 bp, Homo sapiens
  source 1..20: mol_type = genomic DNA
tagcagatgattgagctctg

SEQ ID NO: 273 - RNA, 20 nt, Homo sapiens
  source 1..20: mol_type = genomic RNA
cagagctcaatcatctgcta

SEQ ID NO: 274 - DNA, 20 bp, Homo sapiens
  source 1..20: mol_type = genomic DNA
gttgtccgtagcagatgatt

SEQ ID NO: 275 - RNA, 20 nt, Homo sapiens
  source 1..20: mol_type = genomic RNA
aatcatctgctacggacaac

SEQ ID NO: 276 - DNA, 20 bp, Homo sapiens
  source 1..20: mol_type = genomic DNA
agttgtccgtagcagatgat

SEQ ID NO: 277 - RNA, 20 nt, Homo sapiens
  source 1..20: mol_type = genomic RNA
atcatctgctacggacaact

SEQ ID NO: 278 - DNA, 20 bp, Homo sapiens
  source 1..20: mol_type = genomic DNA
cagcagaaatgacatgtgtc

SEQ ID NO: 279 - RNA, 20 nt, Homo sapiens
  source 1..20: mol_type = genomic RNA
gacacatgtcatttctgctg

SEQ ID NO: 280 - DNA, 20 bp, Homo sapiens
  source 1..20: mol_type = genomic DNA
gagagtgacacatgtcattt

SEQ ID NO: 281 - RNA, 20 nt, Homo sapiens
  source 1..20: mol_type = genomic RNA
aaatgacatgtgtcactctc

SEQ ID NO: 282 - DNA, 20 bp, Homo sapiens
  source 1..20: mol_type = genomic DNA
agagagtgacacatgtcatt

SEQ ID NO: 283 - RNA, 20 nt, Homo sapiens
  source 1..20: mol_type = genomic RNA
aatgacatgtgtcactctct

SEQ ID NO: 284 - DNA, 20 bp, Homo sapiens
  source 1..20: mol_type = genomic DNA
gacctgcttaggtgggtaac

SEQ ID NO: 285 - RNA, 20 nt, Homo sapiens
  source 1..20: mol_type = genomic RNA
gttacccacctaagcaggtc

SEQ ID NO: 286 - DNA, 20 bp, Homo sapiens
  source 1..20: mol_type = genomic DNA
cagagactacaagccaggct

SEQ ID NO: 287 - DNA, 20 bp, Homo sapiens
  source 1..20: mol_type = genomic DNA
agcctggcttgtagtctctg

SEQ ID NO: 288 - RNA, 20 nt, Homo sapiens
  source 1..20: mol_type = genomic RNA
cagagactacaagccaggct

SEQ ID NO: 289 - DNA, 20 bp, Homo sapiens
  source 1..20: mol_type = genomic DNA
gagactacaagccaggcttg

SEQ ID NO: 290 - DNA, 20 bp, Homo sapiens
  source 1..20: mol_type = genomic DNA
caagcctggcttgtagtctc

SEQ ID NO: 291 - RNA, 20 nt, Homo sapiens
  source 1..20: mol_type = genomic RNA
gagactacaagccaggcttg

SEQ ID NO: 292 - DNA, 20 bp, Homo sapiens
  source 1..20: mol_type = genomic DNA
ggcttggccaataacaccat

SEQ ID NO: 293 - DNA, 20 bp, Homo sapiens
  source 1..20: mol_type = genomic DNA
atggtgttattggccaagcc

SEQ ID NO: 294 - RNA, 20 nt, Homo sapiens
  source 1..20: mol_type = genomic RNA
ggcttggccaataacaccat

SEQ ID NO: 295 - DNA, 20 bp, Homo sapiens
  source 1..20: mol_type = genomic DNA
cttggccaataacaccatcc

SEQ ID NO: 296 - DNA, 20 bp, Homo sapiens
  source 1..20: mol_type = genomic DNA
ggatggtgttattggccaag

SEQ ID NO: 297 - RNA, 20 nt, Homo sapiens
  source 1..20: mol_type = genomic RNA
cttggccaataacaccatcc

SEQ ID NO: 298 - DNA, 20 bp, Homo sapiens
  source 1..20: mol_type = genomic DNA
ccaataacaccatccaggta

SEQ ID NO: 299 - DNA, 20 bp, Homo sapiens
  source 1..20: mol_type = genomic DNA
tacctggatggtgttattgg

SEQ ID NO: 300 - RNA, 20 nt, Homo sapiens
  source 1..20: mol_type = genomic RNA
ccaataacaccatccaggta

SEQ ID NO: 301 - DNA, 20 bp, Homo sapiens
  source 1..20: mol_type = genomic DNA
caataacaccatccaggtaa

SEQ ID NO: 302 - DNA, 20 bp, Homo sapiens
  source 1..20: mol_type = genomic DNA
ttacctggatggtgttattg

SEQ ID NO: 303 - RNA, 20 nt, Homo sapiens
  source 1..20: mol_type = genomic RNA
caataacaccatccaggtaa

SEQ ID NO: 304 - DNA, 20 bp, Homo sapiens
  source 1..20: mol_type = genomic DNA
ccagagcatcttacaggcgc

SEQ ID NO: 305 - DNA, 20 bp, Homo sapiens
  source 1..20: mol_type = genomic DNA
gcgcctgtaagatgctctgg

SEQ ID NO: 306 - RNA, 20 nt, Homo sapiens
  source 1..20: mol_type = genomic RNA
ccagagcatcttacaggcgc

SEQ ID NO: 307 - DNA, 20 bp, Homo sapiens
  source 1..20: mol_type = genomic DNA
cagagcatcttacaggcgct

SEQ ID NO: 308 - DNA, 20 bp, Homo sapiens
  source 1..20: mol_type = genomic DNA
agcgcctgtaagatgctctg

SEQ ID NO: 309 - RNA, 20 nt, Homo sapiens
  source 1..20: mol_type = genomic RNA
cagagcatcttacaggcgct

SEQ ID NO: 310 - DNA, 20 bp, Homo sapiens
  source 1..20: mol_type = genomic DNA
tccagagcatcttacaggcg

SEQ ID NO: 311 - DNA, 20 bp, Homo sapiens
  source 1..20: mol_type = genomic DNA
cgcctgtaagatgctctgga

SEQ ID NO: 312 - RNA, 20 nt, Homo sapiens
  source 1..20: mol_type = genomic RNA
tccagagcatcttacaggcg

SEQ ID NO: 313 - DNA, 20 bp, Homo sapiens
  source 1..20: mol_type = genomic DNA
agcatcttacaggcgctgga

SEQ ID NO: 314 - DNA, 20 bp, Homo sapiens
  source 1..20: mol_type = genomic DNA
tccagcgcctgtaagatgct

SEQ ID NO: 315 - RNA, 20 nt, Homo sapiens
  source 1..20: mol_type = genomic RNA
agcatcttacaggcgctgga

SEQ ID NO: 316 - DNA, 20 bp, Homo sapiens
  source 1..20: mol_type = genomic DNA
acaggcgctggatgagctgc

SEQ ID NO: 317 - DNA, 20 bp, Homo sapiens
  source 1..20: mol_type = genomic DNA
gcagctcatccagcgcctgt

SEQ ID NO: 318 - RNA, 20 nt, Homo sapiens
  source 1..20: mol_type = genomic RNA
acaggcgctggatgagctgc

SEQ ID NO: 319 - DNA, 20 bp, Homo sapiens
  source 1..20: mol_type = genomic DNA
caggcgctggatgagctgct

SEQ ID NO: 320 - DNA, 20 bp, Homo sapiens
  source 1..20: mol_type = genomic DNA
agcagctcatccagcgcctg

SEQ ID NO: 321 - RNA, 20 nt, Homo sapiens
  source 1..20: mol_type = genomic RNA
caggcgctggatgagctgct

SEQ ID NO: 322 - DNA, 20 bp, Homo sapiens
  source 1..20: mol_type = genomic DNA
ggcgctggatgagctgctgg

SEQ ID NO: 323 - DNA, 20 bp, Homo sapiens
  source 1..20: mol_type = genomic DNA
ccagcagctcatccagcgcc

SEQ ID NO: 324 - RNA, 20 nt, Homo sapiens
  source 1..20: mol_type = genomic RNA
ggcgctggatgagctgctgg

SEQ ID NO: 325 - DNA, 20 bp, Homo sapiens
  source 1..20: mol_type = genomic DNA
tgagctgctggaggcccctg

SEQ ID NO: 326 - DNA, 20 bp, Homo sapiens
  source 1..20: mol_type = genomic DNA
caggggcctccagcagctca

SEQ ID NO: 327 - RNA, 20 nt, Homo sapiens
  source 1..20: mol_type = genomic RNA
tgagctgctggaggcccctg

SEQ ID NO: 328 - DNA, 20 bp, Homo sapiens
  source 1..20: mol_type = genomic DNA
gagctgctggaggcccctgg

SEQ ID NO: 329 - DNA, 20 bp, Homo sapiens
  source 1..20: mol_type = genomic DNA
ccaggggcctccagcagctc

SEQ ID NO: 330 - RNA, 20 nt, Homo sapiens
  source 1..20: mol_type = genomic RNA
gagctgctggaggcccctgg

SEQ ID NO: 331 - DNA, 20 bp, Homo sapiens
  source 1..20: mol_type = genomic DNA
ctgtgtggccagtcacctgc

SEQ ID NO: 332 - DNA, 20 bp, Homo sapiens
  source 1..20: mol_type = genomic DNA
gcaggtgactggccacacag

SEQ ID NO: 333 - RNA, 20 nt, Homo sapiens
  source 1..20: mol_type = genomic RNA
ctgtgtggccagtcacctgc

SEQ ID NO: 334 - DNA, 20 bp, Homo sapiens
  source 1..20: mol_type = genomic DNA
tgtgtggccagtcacctgct

SEQ ID NO: 335 - DNA, 20 bp, Homo sapiens
  source 1..20: mol_type = genomic DNA
agcaggtgactggccacaca

SEQ ID NO: 336 - RNA, 20 nt, Homo sapiens
  source 1..20: mol_type = genomic RNA
tgtgtggccagtcacctgct

SEQ ID NO: 337 - DNA, 20 bp, Homo sapiens
  source 1..20: mol_type = genomic DNA
actgtgtggccagtcacctg

SEQ ID NO: 338 - DNA, 20 bp, Homo sapiens
  source 1..20: mol_type = genomic DNA
caggtgactggccacacagt

SEQ ID NO: 339 - RNA, 20 nt, Homo sapiens
  source 1..20: mol_type = genomic RNA
actgtgtggccagtcacctg

SEQ ID NO: 340 - DNA, 20 bp, Homo sapiens
  source 1..20: mol_type = genomic DNA
agtcacctgctggatggcct

SEQ ID NO: 341 - DNA, 20 bp, Homo sapiens
  source 1..20: mol_type = genomic DNA
aggccatccagcaggtgact

SEQ ID NO: 342 - RNA, 20 nt, Homo sapiens
  source 1..20: mol_type = genomic RNA
agtcacctgctggatggcct

SEQ ID NO: 343 - DNA, 20 bp, Homo sapiens
  source 1..20: mol_type = genomic DNA
agaggatgtcctcagaggcc

SEQ ID NO: 344 - DNA, 20 bp, Homo sapiens
  source 1..20: mol_type = genomic DNA
ggcctctgaggacatcctct

SEQ ID NO: 345 - RNA, 20 nt, Homo sapiens
  source 1..20: mol_type = genomic RNA
agaggatgtcctcagaggcc

SEQ ID NO: 346 - DNA, 20 bp, Homo sapiens
  source 1..20: mol_type = genomic DNA
cccactacgatgtgcaggtg

SEQ ID NO: 347 - DNA, 20 bp, Homo sapiens
  source 1..20: mol_type = genomic DNA
cacctgcacatcgtagtggg

SEQ ID NO: 348 - RNA, 20 nt, Homo sapiens
  source 1..20: mol_type = genomic RNA
cccactacgatgtgcaggtg

SEQ ID NO: 349 - DNA, 20 bp, Homo sapiens
  source 1..20: mol_type = genomic DNA
ggcccactacgatgtgcagg

SEQ ID NO: 350 - DNA, 20 bp, Homo sapiens
  source 1..20: mol_type = genomic DNA
cctgcacatcgtagtgggcc

SEQ ID NO: 351 - RNA, 20 nt, Homo sapiens
  source 1..20: mol_type = genomic RNA
ggcccactacgatgtgcagg

SEQ ID NO: 352 - DNA, 20 bp, Homo sapiens
  source 1..20: mol_type = genomic DNA
gtctcacctgcacatcgtag

SEQ ID NO: 353 - DNA, 20 bp, Homo sapiens
  source 1..20: mol_type = genomic DNA
ctacgatgtgcaggtgagac

SEQ ID NO: 354 - RNA, 20 nt, Homo sapiens
  source 1..20: mol_type = genomic RNA
gtctcacctgcacatcgtag

SEQ ID NO: 355 - DNA, 20 bp, Homo sapiens
  source 1..20: mol_type = genomic DNA
tctcacctgcacatcgtagt

SEQ ID NO: 356 - DNA, 20 bp, Homo sapiens
  source 1..20: mol_type = genomic DNA
actacgatgtgcaggtgaga

SEQ ID NO: 357 - RNA, 20 nt, Homo sapiens
  source 1..20: mol_type = genomic RNA
tctcacctgcacatcgtagt

SEQ ID NO: 358 - DNA, 20 bp, Homo sapiens
  source 1..20: mol_type = genomic DNA
ctctctggattttgaaaaac

SEQ ID NO: 359 - DNA, 20 bp, Homo sapiens
  source 1..20: mol_type = genomic DNA
gtttttcaaaatccagagag

SEQ ID NO: 360 - RNA, 20 nt, Homo sapiens
  source 1..20: mol_type = genomic RNA
ctctctggattttgaaaaac

SEQ ID NO: 361 - DNA, 20 bp, Homo sapiens
  source 1..20: mol_type = genomic DNA
acaagccaggcttggccaat

SEQ ID NO: 362 - DNA, 20 bp, Homo sapiens
  source 1..20: mol_type = genomic DNA
attggccaagcctggcttgt

SEQ ID NO: 363 - RNA, 20 nt, Homo sapiens
  source 1..20: mol_type = genomic RNA
acaagccaggcttggccaat

SEQ ID NO: 364 - DNA, 20 bp, Homo sapiens
  source 1..20: mol_type = genomic DNA
agccaggcttggccaataac

SEQ ID NO: 365 - DNA, 20 bp, Homo sapiens
  source 1..20: mol_type = genomic DNA
gttattggccaagcctggct

SEQ ID NO: 366 - RNA, 20 nt, Homo sapiens
  source 1..20: mol_type = genomic RNA
agccaggcttggccaataac

SEQ ID NO: 367 - DNA, 20 bp, Homo sapiens
  source 1..20: mol_type = genomic DNA
caggcttggccaataacacc

SEQ ID NO: 368 - DNA, 20 bp, Homo sapiens
  source 1..20: mol_type = genomic DNA
ggtgttattggccaagcctg

SEQ ID NO: 369 - RNA, 20 nt, Homo sapiens
  source 1..20: mol_type = genomic RNA
caggcttggccaataacacc

SEQ ID NO: 370 - DNA, 20 bp, Homo sapiens
  source 1..20: mol_type = genomic DNA
tggccaataacaccatccag

SEQ ID NO: 371 - DNA, 20 bp, Homo sapiens
  source 1..20: mol_type = genomic DNA
ctggatggtgttattggcca

SEQ ID NO: 372 - RNA, 20 nt, Homo sapiens
  source 1..20: mol_type = genomic RNA
tggccaataacaccatccag

SEQ ID NO: 373 - DNA, 20 bp, Homo sapiens
  source 1..20: mol_type = genomic DNA
agagcatcttacaggcgctg

SEQ ID NO: 374 - DNA, 20 bp, Homo sapiens
  source 1..20: mol_type = genomic DNA
cagcgcctgtaagatgctct

SEQ ID NO: 375 - RNA, 20 nt, Homo sapiens
  source 1..20: mol_type = genomic RNA
agagcatcttacaggcgctg

SEQ ID NO: 376 - DNA, 20 bp, Homo sapiens
  source 1..20: mol_type = genomic DNA
gcatcttacaggcgctggat

SEQ ID NO: 377 - DNA, 20 bp, Homo sapiens
  source 1..20: mol_type = genomic DNA
atccagcgcctgtaagatgc

SEQ ID NO: 378 - RNA, 20 nt, Homo sapiens
  source 1..20: mol_type = genomic RNA
gcatcttacaggcgctggat

SEQ ID NO: 379 - DNA, 20 bp, Homo sapiens
  source 1..20: mol_type = genomic DNA
tcttacaggcgctggatgag

SEQ ID NO: 380 - DNA, 20 bp, Homo sapiens
  source 1..20: mol_type = genomic DNA
ctcatccagcgcctgtaaga

SEQ ID NO: 381 - RNA, 20 nt, Homo sapiens
  source 1..20: mol_type = genomic RNA
tcttacaggcgctggatgag

SEQ ID NO: 382 - DNA, 20 bp, Homo sapiens
  source 1..20: mol_type = genomic DNA
tacaggcgctggatgagctg

SEQ ID NO: 383 - DNA, 20 bp, Homo sapiens
  source 1..20: mol_type = genomic DNA
cagctcatccagcgcctgta

SEQ ID NO: 384 - RNA, 20 nt, Homo sapiens
  source 1..20: mol_type = genomic RNA
tacaggcgctggatgagctg

SEQ ID NO: 385 - DNA, 20 bp, Homo sapiens
  source 1..20: mol_type = genomic DNA
aggcgctggatgagctgctg

SEQ ID NO: 386 - DNA, 20 bp, Homo sapiens
  source 1..20: mol_type = genomic DNA
cagcagctcatccagcgcct

SEQ ID NO: 387 - RNA, 20 nt, Homo sapiens
  source 1..20: mol_type = genomic RNA
aggcgctggatgagctgctg

SEQ ID NO: 388 - DNA, 20 bp, Homo sapiens
  source 1..20: mol_type = genomic DNA
agctgctggaggcccctggg

SEQ ID NO: 389 - DNA, 20 bp, Homo sapiens
  source 1..20: mol_type = genomic DNA
cccaggggcctccagcagct

SEQ ID NO: 390 - RNA, 20 nt, Homo sapiens
  source 1..20: mol_type = genomic RNA
agctgctggaggcccctggg

SEQ ID NO: 391 - DNA, 20 bp, Homo sapiens
  source 1..20: mol_type = genomic DNA
gggacctggagaccctgccc

SEQ ID NO: 392 - DNA, 20 bp, Homo sapiens
  source 1..20: mol_type = genomic DNA
gggcagggtctccaggtccc

SEQ ID NO: 393 - RNA, 20 nt, Homo sapiens
  source 1..20: mol_type = genomic RNA
gggacctggagaccctgccc

SEQ ID NO: 394 - DNA, 20 bp, Homo sapiens
  source 1..20: mol_type = genomic DNA
tggagaccctgccccgctta

SEQ ID NO: 395 - DNA, 20 bp, Homo sapiens
  source 1..20: mol_type = genomic DNA
taagcggggcagggtctcca

SEQ ID NO: 396 - RNA, 20 nt, Homo sapiens
  source 1..20: mol_type = genomic RNA
tggagaccctgccccgctta

SEQ ID NO: 397 - DNA, 20 bp, Homo sapiens
  source 1..20: mol_type = genomic DNA
agcactgtgtggccagtcac

SEQ ID NO: 398 - DNA, 20 bp, Homo sapiens
  source 1..20: mol_type = genomic DNA
gtgactggccacacagtgct

SEQ ID NO: 399 - RNA, 20 nt, Homo sapiens
  source 1..20: mol_type = genomic RNA
agcactgtgtggccagtcac

SEQ ID NO: 400 - DNA, 20 bp, Homo sapiens
  source 1..20: mol_type = genomic DNA
tggccagtcacctgctggat

SEQ ID NO: 401 - DNA, 20 bp, Homo sapiens
  source 1..20: mol_type = genomic DNA
atccagcaggtgactggcca

SEQ ID NO: 402 - RNA, 20 nt, Homo sapiens
  source 1..20: mol_type = genomic RNA
tggccagtcacctgctggat

SEQ ID NO: 403 - DNA, 20 bp, Homo sapiens
  source 1..20: mol_type = genomic DNA
ccagtcacctgctggatggc

SEQ ID NO: 404 - DNA, 20 bp, Homo sapiens
  source 1..20: mol_type = genomic DNA
gccatccagcaggtgactgg

SEQ ID NO: 405 - RNA, 20 nt, Homo sapiens
  source 1..20: mol_type = genomic RNA
ccagtcacctgctggatggc

SEQ ID NO: 406 - DNA, 20 bp, Homo sapiens
  source 1..20: mol_type = genomic DNA
tgctggatggcctagaggat

SEQ ID NO: 407 - DNA, 20 bp, Homo sapiens
  source 1..20: mol_type = genomic DNA
atcctctaggccatccagca

SEQ ID NO: 408 - RNA, 20 nt, Homo sapiens
  source 1..20: mol_type = genomic RNA
tgctggatggcctagaggat

SEQ ID NO: 409 - DNA, 20 bp, Homo sapiens
  source 1..20: mol_type = genomic DNA
aggatgtcctcagaggcctg

SEQ ID NO: 410 - DNA, 20 bp, Homo sapiens
  source 1..20: mol_type = genomic DNA
caggcctctgaggacatcct

SEQ ID NO: 411 - RNA, 20 nt, Homo sapiens
  source 1..20: mol_type = genomic RNA
aggatgtcctcagaggcctg

SEQ ID NO: 412 - DNA, 20 bp, Homo sapiens
  source 1..20: mol_type = genomic DNA
gcaagaacctttccaatggg

SEQ ID NO: 413 - DNA, 20 bp, Homo sapiens
  source 1..20: mol_type = genomic DNA
cccattggaaaggttcttgc

SEQ ID NO: 414 - RNA, 20 nt, Homo sapiens
  source 1..20: mol_type = genomic RNA
gcaagaacctttccaatggg

SEQ ID NO: 415 - DNA, 20 bp, Homo sapiens
  source 1..20: mol_type = genomic DNA
aatgtcagcagaacccaagg

SEQ ID NO: 416 - DNA, 20 bp, Homo sapiens
  source 1..20: mol_type = genomic DNA
ccttgggttctgctgacatt

SEQ ID NO: 417 - RNA, 20 nt, Homo sapiens
  source 1..20: mol_type = genomic RNA
aatgtcagcagaacccaagg

SEQ ID NO: 418 - DNA, 20 bp, Homo sapiens
  source 1..20: mol_type = genomic DNA
cgtgccagtgcctgcctggc

SEQ ID NO: 419 - DNA, 20 bp, Homo sapiens
  source 1..20: mol_type = genomic DNA
gccaggcaggcactggcacg

SEQ ID NO: 420 - RNA, 20 nt, Homo sapiens
  source 1..20: mol_type = genomic RNA
cgtgccagtgcctgcctggc

SEQ ID NO: 421 - DNA, 20 bp, Homo sapiens
  source 1..20: mol_type = genomic DNA
gtaaaagctacggcacctgc

SEQ ID NO: 422 - DNA, 20 bp, Homo sapiens
  source 1..20: mol_type = genomic DNA
gcaggtgccgtagcttttac

SEQ ID NO: 423 - RNA, 20 nt, Homo sapiens
  source 1..20: mol_type = genomic RNA
gtaaaagctacggcacctgc

SEQ ID NO: 424 - protein, 823 aa, Homo sapiens
MGGRVFLVFLAFCVWLTLPGAETQDSRGCARWCPQDSSCVNATACRCNPGFSSFSEIITTPMETCDDINECATLSKVSCGKFSDCWNTEGSYDCVCSPGYEPVSGAKTFKNESENTCQDVDECQQNPRLCKSYGTCVNTLGSYTCQCLPGFKLKPEDPKLCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITYMGLSVSLLCLLLAALTFLLCKAIQNTSTSLHLQLSLCLFLAHLLFLVAIDQTGHKVLCSIIAGTLHYLYLATLTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 425 - DNA, 61372 bp, Homo sapiens
  source 1..61372: mol_type = genomic DNA
tacttacttattttctgagacagagtctcactctgtggcccaggctggagtacagtggcgagagctcactgcaaccttgaactcctgggctcaagagatcctcccacctcagccccgtgagtagctgggattccaggcacatgccatcactcctggctaatttttgcattttttggtagagatggagttttaccatgttgcccaggctggtctcgatcccctgggctcaagggatcctcccgcctcggcctccaaaagtgctgagatcacaggcatgagccactctgcctggccgattttatcttccttgatacaaactcttttcagaattctctttgtttttgctttggtccctgataaaatcacatgtgaatataaccgcaaaacaccacatgcctgcattacatggcgttacgttaaacaaccatcattttggctgggcgcagtgactcatgcccgtctctactaaaaatacaaaaaaattagcctggcgtggtggtgtgcacctgtaatcccagccacttgggaggctgaggcaggagaatggcttgaacgcggggggcggaggttgcagtgagccgagattgtgccactgcactcctgcctgggcgacagagcaagagactctgtctcaagaaaacaaaacaaaacagccatcattttaggaaacattcatatgtggaaactactatgtgtattgttcacaatttgcgggaataaagaactttgctttgcaaaaaatatgtaaattaaaaaataaaagggaaggaaggcatacccatgagcctccctcatcaccaagaactgagacatgaccttgttcccatgctcccatctttatcttgctgattaccaccactctgaatattctttgcaccttattcctttgctcagttacttatttttttattttatttatttttgagacagggtcttgctctgtcacccaggctggagtgcagtggtatgatcacagctcactgccacctcgaattcctgggctcaagccatcctcccacttcagcctcctgagtagctgggaccacaggtgcgagtcaccacacctggcgaattttttatttttattgtagagatggaggtctcattatgttgcccaggatggtctcgaactcctgggctcaagcaatcctcccgtctcagcatccccaagtagctgggattacgagcatgaaccactgcgcccggcccagccttgtttatttttatttatttatttatatttatgtatttttattttattttattttatttagacggagtctcactcttgtctcccaggctggagtgcagtggcgcaatctcagctcactgcagcctctgcctcccgggttcaagtgattctcttgcttcagcctcccaagtagctgggattacaggcacccaccaccatacccggctaatttttgtacttttagtacagacggggtttcaccatgttggccaggctggtcttgaactcctgacctcaggtgatctgcccacctcggcctctctgggattacaggcatgagccaccgcgcccggcagccttgattattttttaaagtcttcttgcatatgtttgtatcctgaaaccaaatcttttattatctttgtatctcagcttttttgcaaggtggctttttcctatggggctttggagactttttcaagattctagtatggaatctgtcatgctgcaatttaagatttttttctcctgacttcactcagtttttaaatttaaaaaaggagtgtggctaggcatggtggcatgcacctgtaaccccagcattttgggaggctgaggtgggcggatcacttgagggtcaggagatcgagaccagcctggccaacatggtgaaaccccgtctctactaaaaaattagctgggcgtggtggtgtgtgcctgtaatcccagcttctagggaggctgaggcaggagaattgcttgaagctgggaggcggaggttgcagtgagccgagatctcgccactgcactctagcctgggtaacagggcaagattccgtccccctcacacacacacaaaaaaagtgaaaagttagccaggcatggcaacatatgcctgtagtcccagctacttggtaggctgaggcaggaggatcacataagcccaggacgtgcaggttgcctgaagccataattgtgccaccgccactgtgctccagcgtgggcaacagagaccctgtctctaaaataataacaataaattatactaatataattaataataccattattattgttggttggataatcttgggttgcttcttttttttttttttttttttttgaaacggagtctggctctatcgcccaggctggagtgcaataacgcgatctcagctcactgcaacctacatctcctgggctcaagcaattctcctgcttcagcctcccaagcagctgggattacaggtaatcttgggttacttcttagctgagcaacctggggccaggggccccaaacccccaggttccagttccccttttgacaaatgaggaccatgttcgcaccccccatcattgcagaggttgtccagaagctgcaaaataggtcagcaggactgagacttctggacaaagagaaggcggagaaacataggcagacaccccggcagggaccagatcagcgaggctgaaggaggcttcctttagctgggcggcagcggagtggagtggcaggagtactactaatttgcatggtttaccatatagggccccactcagcacaggctctgtgctggtgctctgcagagaaaaagacaaaagtcactcagggggctgacagcctaatgcggctcgagggcagatggttgctagggaagaagggagatggtgagaagtattggagagactcaagaaagccgccaggaatggtgactcacgcctgtaatcccaacgctttgggaggctgaggcgcgtggatcatttgaggacaggagttcgagaccagcctggccaacatggtgaaaccctgtctccactaaaaatacaaaaattatctagatgcgatggcaggtgcctataatcccagctactcgggaggctgaggcaggagaattgcttgaacccgggaggcagagattgcagtgagacgagatagcaccactgcactccggcttgggtgacagagcaagacttcgtctcagaaaaaagtaataataaataataataataataataaaataaaaatttaaaaagaatgtttcaggccgggcattgtggttcacgcctgtaatcccagcacttttgggatgccaaggcaggcagatcacttgaggccagaagttcgagaccagcctgaccaacatagcgaaactccatctctgctagaaatacaaaaattagccaggcgtggtggcttgcacctgtaattccagatacttgggaggctgaggcaggagaatctcttgaacccaggaggcagaggttgctgtgagctgagatcacatcaccacactccagcctgggtgacagagcgagactctgtctcaaaataaataaatgaatgaacaaataaataaaataaaataaaaatacaaaaattagccaggtgtggtggtgggcacctgtagtccgagctactctggaggctgaggcaggagaatcgcttgaacccgggaggcggaggttgcagtgagtcgagatggcgccactgtactccaacctgggcaacaccagagtctgtctctaaaatacagtaaaataaaattaaattaagaagagtcgatgatcaccatgaaggggtgtagggacaatccctggcatcaggaagtgccactacctccctgagattctgcagacgccagctgtctgacctgcccgtctgatctgcagagaagggacgttgagccctggggttctagctggagtttctggctttctgagaaaaaaaaggcaaaatccctccagacctggtctggccctctctccttccagcttctgctggtgttcactcccctcccccacagcccaggccacccccacaggagcccgggacaggaaggggtaaattctaggactgtggatgtttgattattcccaggacaatagttctctgacttcagtcattagtgacttttttctccatcgctgcaatctccccctttgaacaactgaactatattttccttagcattcatcacttctgcatcttgaatttactcatgtaacaaggagacttgatttcggcatcctcactgaaaaaccctcctcattgccccagatagtagggaaacctaaaaataaatataaatatgaggaacacaaaaccaagtccttgaattctggctggatacagacacctgctaagcctcagggtcacggaggcgctaaagacatgttagtgcccagtggagactttcccctgggctcatcgcagggactccagggctgggctgccggcctgtggatctgagtctcagctgggtgagtcacttgtgcctcagtttcctcctctgtcaaatgggaatgacaacatcattgcagatgttttaaggagtaaatgagttaatatagattaaaaaaaaaaagcacttaggaccagacctggtacaaattaagtgctccataaataatagcattttcttggcaggatagcgagatctcgtctctacaaaaaaaaaaaacacttaaggagaaaactagccagacgtgatggtgcacagctgtagtcccagctcctcaggaggctgaggcaggaggatcccttgagccctgaagtttgaggctgcagtgagctgtgatggtgaccctgcactccagcctgggtgacagagtaagatctcttaaaaatatatatagcgttttaaaaaatgattacgtacctcaagagacaagctaatcatatgatttatccagaaaggaaagaaccctcctatcacatgattctctgttgattaacgaagtgcccaggggcctctgaaatataagtttcccacaggaggaaactggcttagaaagaccagaaagatccggggggtctgctctgtgcggtgaagcttctcttcttggcacctgcctggcatcggaagagggccccttctccctccctgggcttttatgtggacactgtaatgcctcagttttctttctttctttttgtttttgacacagggtctcactctatcacccaagctggagtgcagtggcaccatcttagctcaccgcagcctcaaactccccagctcgggtgattcttcctgcctcagcctcctgagtagtagcagctgggaccacagacgtctgccaccaagcccaggtaattttttttctttttgaagaaacagagctttgccatgttgcccaggctggtctcaaactcctgagctcaagcgatcctcctgcctcggcctcccagagtgctgggatcacaggtgtggagaaataggaacgcttttacactgttggtgggaatgtaaattagttcaaccattgtggaagatagtgtggtgatttctcaaggatccagaactagaaataccatttgactcagcgatcccattgctgggtatatacccaaaggattataaatcatgctactataaagacatatgcacatgtatgtttattgcggtactattcacaatagcaaagacctggaatcaacccaaatgtccatcaatgatagactggattaagaaagtgtggcacatatacaccagggaatactatgcagctataaaaaaggatgagttcatgtcctttgcagggacacagatgaagctgaaaaccatcattctcagcaaactaacacagaaacagaaaaccaaacaccacatgttctcactcataggtgggaactgagcaatgagaacacctggacacagggcgggaaacatcacacactggggcctgtcggggggtgggggcctgggggagggattgcattaggagaaatacctaatgtaaatgacgagttgataggtgcagcaagccaacatggcacatgtatgcctatgtatcaaacctgctcgttgtgcacatgtaccctagaacttaaagtctaaaaaatttaattaattaattaattaattaatttaaaaagaaagaaagaaaccgagcttcgccatgttgcccaggctggtctcaaactcctgagctcaagcgatcctcctgcctcgccctcccagagtgctgggatcacaggcgtgagcctctgtacctggcctcagtttcctcatctggaaaacagcaggggaagatactgccagctgggacgcaggctctgccaactctggcatgccaaagctctgggcacagggtgagccctaccaaagtgggagcccagagctatttctgacaaggcccctgcgtgcctgcccagggaagagccaagtggccccaggcgagaccgccccgcccggctgccacaccttcccctgcacagcagccagcctggaacacacagaggccagagggttggtcagagccacatggtggaaactccagcagagggatttttaaagcaggttgcacttcattcttgctgagcgagtacacgtgtgtgtgtgtgtgtgtgtgtgtgtgtgtgtgtgtgtgcagcgcccctggttctgtgtttttattgtgctttcctgctggcttccagctgcaccgccagttccggggagggccctgggccagcggctgtccgccccccctcctttataaagtcctggcctcgggacagcccgcacagctgcccagcctgcggagacgggacagccctgtcccactcactctttcccctgctgctcctgccggcagctcagctggaaccatgggaggccgcgtctttctcgtctttctcggtaagtactttggggccccgctgggggtggtagcgaggaagctccagcgggaccccttggatgcgtctgagaaacgggaggcgccgctggtgtctgtggggccggcgatggagaaagccacatggtcaagcaggcactcacgggcacacggcgagaggggctggtggtgcttgggcacccccacaccccggcaggaactccagggtgctctcggagcctgctaccctgcccggagggatcctcaagctttctactggagccagtttggcatctgctccataagaggggaaactgaggcacgggggaaggaaaggggcaaaaagtgagtgctaggaccccagagccactgctcttaatatagcgcatagaaagacatgggcggggggcggggaaaatagaggccaggacttctccactgcagggtacccccagcttctcccttctttctctctctctctctgtctttctccttctgtctctctttcagtctctctatctccctaatctccgttttctctctacctctgcctctttgtctctctctctggtgaacaacaaggagatccctaggtccccatctctaaagcccctggtgacctgaggagggtcccccaggacatgccttggaaaggggaacttcctgagtgctgggaaccgcagcgccacatgcaccccaacaggcggccacagcgttttcctgtgggaggggcgcccggggtgaaatatcaccactgggtttcaggatgggccgccagggctggcggcggttgtggtgtgtgtgcacctgtgtccctgaggaagcatgcggttccagggcatcttagagcctgggggatccagcgccatcatttttttttttttttttctgaggcggagccttgctctgtcgcccaggctggagtgcagtggcacgatctcagcttgctgcaacctccgcctcctgggttcaagcgattctcctgcctcaatctcccaagtagttgggtaacaggagtctgccaccacgcccagctaatgttttgtatttttagtagagacggggtttcaccatattgggcaggctggtctcgaactcccgacctcagatgatctgcccaccttggcctcccaaagtgctgggattacagtcccgagccaccgtgcctgaccatgccttcatttttatctaaagaaatgaagatggcagtaaatgctcaggcacaccagacagcccccaagtcagaagagcggcagctggagctgagacccccaccaggctcatggccctttcctactcctcagttccttaaaccccacccccaagccaagctagggaggctgaggcaggaggatcgcttgaggccaggagttcaagatcagcctgggcaacagagcaagactctgtctctaaaataactttttaaattatttttaatttttttggccaggcgtgatggctcatgcctgtaatcccagcactttgagaggccaaggcgggcagatcacctgaggttaggagttcaagaccagcctggccaacatggtgaaaccccatctctacttaagaaatacaaaaattagctgggcatggtgggtcatgcctgtagtcccagccactcaagaggctgaggcaggagaaccgcttgaacccaggaggtggagattgcagtgagccaagattgcaccactgcattccagcctgggcaacaaagggagactccatctcaaaaaaaaataaataataaaaataaataaatcattttaaaatttttgagctgggagccgtggctcacacctgtaatcctagcactttgggaggccaaggccggcagatcacctgaggtcaggagttcgagaccagcctggccaatatggtgaaaccccatctcactaaaaatacaaaaaattagccaggcaccatggtgggtgcctgtagtcccagctatttgggaggttgaggcaggagaattgcttgaacccaggaggtagagattgcaattagccgagatcgcaccattgcacttcagcctgggcgacagagcaagaccctgtctcaaaaagaaagaaagaaaatttaaaatatggcggtactcacctgtggtcccagctactcgggagggtgaggcagggggatcgcttgagcctaggaattggaggctgcagtgagctataatcgcatcactgcaccccagcctgggtgacccagagagaccctgtgtcaaacaaaacaaaacaaaacccacccccatgaggacagggtgacagactctctggctttgagcatccgagtgggacgtgacatctgctcactctgtttgaacgatgggcaggatctgagcctgagaggggtcagcatcctcctcccacaaagtgcatcacccttacgcctcctttcccaccctgggatcccctctgaccccctttcctttctctgttgcagcattctgtgtctggctgactctgccgggagctgaaacccaggactccaggggtgagtctgctgggaagcagaaagcacagtccacagccagagcctggggagggtcctggacccccgcccagcccccttcagcccagggaaagagagggctcgtgcacgggaaactcagcgctctgccccatctcccccagtgccccctttttgtgtattcccttacccctcaccttctgaccgtgctccctgctcttgcaggctgtgcccggtggtgccctcaggactcctcgtgtgtcaatgccaccgcctgtcgctgcaatccagggttcagctctttttctgagatcatcaccacccccatggagacttgtgacggtacagaggcttgagggcagcgcaggggacatctgcgattatgaggcatagcccagtgccagtgggggacagaggttgttgtgaggggccacagccttaccttccagactatcatcatggccagagaaaagagaaagagggcaggtgtggtggctcccagcactttgggaggcagaggcgggtggatcatttgaggccaggaattcaagaccagcctgggcaacatggtgagaccctgtctctacaaaaaatacacaaaatagccgcgcatggtggcacacatctggagtcccagctactcagaagactgaggcgggaggattgtttgagcccaggagtttgaggttgcagtgagctatgattgcaccattgcactctagcctgggcaacagagtgagatcctgtctcaggaaaaagaaagaaacaaagaaagaggccaggtgtggtggctcatgcctgtaatcccagcactttgggagggattgtctgggcaacaggagcaagaccccatctcaaaaaaaaaaaaaaaagacagaaagagagaaggagagaaagaaaagagaagaaaagaaaaagagagagagggagggagggaaagaaaaaggaaaggagggagggagggagggaggaaagaaggaaggaaggaagggaggaaaaaggaaaaaatatgaagggggagaagtaagaagtgaataggcatggcttcctggagagagagaagctgcgtgctcaggaatctggagtctgtgcctcagtttaccgttaagactgggaagggggtacattctggccataggtttttttttctcccttatttttcttttttctttttttttttgttttttttttttttttttgagatggagtctcactctgtcgctcaggctgaggagtgcagtggcgcgatctcggctcaccgcaacctccacctcccaggttcaagcgattcttctgcctcagcctcccgagtagctgggattacaggcatgcaccaccatgcccggctagtttttgtatttttagtagagatggggttttgccctgttggcaggctggtctcgaactcctgacctcaagtgatctgccggcctcggcctcccaaagtgctgggattacaggtatgagccactgcacccggctgccatggggtttaaatttctacaaaaagactggggatggggatatggcgcttcctgtccatcccccacctcagcactgtccactgtgtccaaatctaggatgccagccagagaactgagatcaagtgtggtcttcaggaatggcctgctccagctgcagcatagcctgtgcagtgcaacttgagatcctttccaaaagatactaagcagcaggccccatgttggggagtcaggacgggggtctctgccctgacggaactcacatctttgggagatgacctcctgtcctgttgtgttccagacatcaacgagtgtgcaacactgtcgaaagtgtcatgcggaaaattctcggactgctggaacacagaggggagctacgactgcgtgtgcagcccaggatatgagcctgtttctggggcaaaaacattcaagaatgagagcgagaacacgtgtcaaggtaagaaccaccccacatcctccatcaccactgtccatgaggtttggggtcaccagagctgtttctgcagcatccagggagcaggtacccaagtataggttcagttcctggagtctgagatgagacaggtgtgcatgtacctgtttcgccagcccagagaggcagcgtggtgtgaaggccaagggaaaactctgggaccagctacctgggcccaaatcctggctttgccacctgttacctaaatgatattaccaacatcactactgctcttagagccttggtttacccatctataaaatgaggatgataataatggcctactcctatgccttctaggaggaccaaggaaagaataggtgcttctaacagagcccagcacacagcaggtgctacaggcatcttcattctttaaacgttattatgcactgtaatcccagcactttgggaggccgaggcggatggatcacggggtcaggagatcgagactatcctggccaacatggtgaaaccctgtctctactaaaagtacgaaaattagccgggtgcagtggtgggtgcctgtaatcccagctactcaggaggctgaggcaggagaatcacttgaaccagggagtcggaggtttcagtgagcggagatcgtgccactgcactccagcctggtgacagtgcaagactccatctaaaataaataaataaataaataaataaataaataaataagcaatggatgccttttaggagttgttcagacaagctgggctcctgtggggccacactcctggctggcactcagaagaaatctatttcgacacagcctgcctcatcccttgccatggtcttattccacactgcagctccagcccatgagaagccaggcccccatgcatcactctgctttgccttctttccagtgggaactagttgggtgcgggagggagcttcagacatctgcaaggccagatgggaaccagtagcaggtggacagagaagggagattaacacagagtacacagagtgggaagggaaggtgagcaaaggcaggaaatgaagaagctgaggctgagccaagggtcccaggggacattaagggggttcagcagacctgccctgctgtaaagagaaaagtggcagtgctcctgttcccagccctgtccctcctgttctccctgcagtcagtagagcctccctggtgggaggaaagtgaggacagagcagaggtgtacgttctgagagcacgcagctgcatagagcccagcaggggggctggttcacaggggtcccctgtgaacagatgctacattgcatgcacatatcttgaccaaaggacagagccttcctgtgaattagaaaaaggactctccgggccaggcacagtggctcacgcctgtaatcccagcactttgggaggctgaggcgggtggatcacctgaggtcaggagttcaagaccagcctgacaaacatagtgaaaccccatctctactaaaaaatacaaaagttagccaggcatagtggtgagcacctgtaatcccagctactcaggaggctgaggcaggagaatcacctgaacccagaggcagaggttgcagtgagccaggatctcaatattgcactccagcctgggtgacagagcaagactttgtctcaaaaaaaaaaaaaaaaaaaaaaaaaaaagaaaagaaagaaacaaaaagaaaaagagaaacagaaaagcctctcctctccaggtagacacagccccatttggggaatggagaggttttcagcctctgctcaccgccttggctgggcatccatatgtagtcagcaacctacacaactgtacgtggtggtcatgggtccttgtcagtgagagcctctgttttagtctatgttgtgttgctataaaggaatacatgaggctggatcatgtataaagaaaagaagctgatttggctcatggttctgcaggccatgtaagacacatggctccagcatctgcttctggtgagggccttagggagcttccactcttggcagacgggaaaggggaagctggcatcccatggcaagaggaaggaagcaagagagaagggaggagataccaggctttttccaacaagcagttttcatgggaactaacaaggcgagaactcactcatgacccaaggacagcaccgagatgttcatgagggatctgcactcaggacccaaacacctcccacgaggctccatctccaacactggggatcacatttcaacgtgagatttggaggggacaaatatccaaactgtatcaccgcatgcacaggtggtgtttgtaattattgaggaggtgtgttgccatgttagaaacaggccagcactggccgggcgcagtggctcatgcctgtaatcctagcactttgggaggccgaggtgggcggatcacaaggtcaggagatcgagaccatcctggctaacatggtgaaaccccgtctctactacaaatacaaaaaaattagctgggcgtggtggtgggtgcctgtagtcccagctaatcagagactgaggcaggagaatggcatgaacctgggaggcggagcttgcagtaagccgagatggcaccactgcactccagcctggacgacagagcgagactccgtctccaaaaaaaagaaaagaaaaggagagaagagaagagagaaaagaaaagaggccaacactgagagagggggcttagttggtggataggcagagacacagatgagcaggaatgaagtgggagagggctctcatggaccccatccgcacagaacagcctgctagttcggagcaggacctgcctcttgaagcctcagctcagggaagggctccgtggggccctgtctgcatccccctcacccctcagcattcttgttttttttgtctacctggtgtcacaggcctcataggcaggagacctggggccactccctcagctccttgccccacttactccaggcatcacagggccgtcccaaacacctggtcacctgtcgctatccaaacctcaaggttcagaaccctccacagagggtcacttccaaaagctcagaacatccccctgcccatctggcatcttatttgtttgttttgttttgttttattagtaaagatggggggggggtgtctcaccatgttgtccaggctggtctcgaactcctggcctcaagtgatccgcccgtctcagcctcccaaagtgctgggattgcaggcgtgagccaccatgtccgacctggtatcttatttgtacaaaacaaaaagggggctggcactcaggatattcgatgagctcactgcgtggtgcattccagcgcagtatccattctctctgctatcatcagcgactgccaggatgagacagtctccacacagctctgctcacctggaactctctctgatcccccagctcatcatggacccaggcaccctgtccagaaatgaaggaggagggagatttgaaggcagggccactgtccccagtcgaacctgccactggccaacaaccccacctggagaagatgcctcgtggagccctgacctctttttctgccactcagtctctgtccctttcactgagcgagactgccctgacccttcctgatagttttggtccacctgagtgcactggaggatgttgtggctaacggtgggcacagtgacatggactccccatacttggagagttgagtgaatggaagaggcactagtgtggacccaatggcaagagaggttaacatcagggccattgctccaagcctgatcctctcccaggaggtcaacccagatcccatgggagtggaaccattcagaaataggtctgtgtccctggtggaatgccaagagatgagatgacagatacacctccatctttgcccatcagcatctgtgcccctctccagccactaaaccttcacccagggccagtctttctcagctatggattctcactacatcctgccatttccaagacaccttcctgaggatcaaaaaaaggcaccttcacagccctggggcaacccagctgtatgtcggtccccactgtccaccctggcctgggacccactgtccaggcagccctggcctatgcagcaccagactcatgctcccctggggactggctcaggggccagtcttggtccatgtgcctggagctgggctctcagagctagtgtggatgggagagcaccatcctgctagggccaccgggattctgataactcagccagtgagcagctgtcctggggaaatcaaggccaccagaagcagatgctggagccatgcatcttacacagcctgcagaaccatgagccaaataaacttcttttcttgcaacacaaaacggactaaaccagagggttctcactgagaaggacccatgaccaccatgtacagttgcataggctgctgactgcatatggatgcccagccaagagggtgagcagaagctgaaaacctgcctactctccattcctcaaatatgactgtgactacttgaagaggagatgcctttttccaattcacaggaaggctctgtcctttggcccaggactgccactgggaaccaagacaggtcctacctgggcaagcagcatgccccctgcacttggtcccaagaaaatcccaccctgacctttgtcctgaatcctcaggctgggacatccctgccccagctgggagctgcccagggaggcaggggagatggggaggactcagatgcttccagccatatctgaacaacagaaggcctgggccaccatgctcaccatgggacacctctgtgtggattagaaaagggaggagatggccaggcatggtggctcatgcctgcaatcccagcactttgggaggccgaggtgggctgatcacgaggtcaggagatcaagaccatcctggctaacacggtgaaaccccgtctaatactaaaaatacaaaaaattagccgggcatggtggcgggcgcctgtagtcccagctattcgggaggctgaggcaggagaatggcgtgaacccgggaggcggaggttgcagtgagttgagatcaggcaattgcactccagcctgggtgacagagcaagactccgtctcaaaaaaaaaaaaaagaaagaaagaaaagaaaagggagcagacagtggtgccttccagatgcttccgtgccaggaaacatggcggaccctcagccccgaaagctgctcacagctacgggcattcttcaccctctccttcctcttgcagatgtggacgaatgtcagcagaacccaaggctctgtaaaagctacggcacctgcgtcaacaccctcggcagctacacgtgccagtgcctgcctggcttcaagctcaaacctgaggacccgaagctctgcacaggtagaggccccaggaagacgctgtgaggctggacgggagctggggatggagctgagtcaggtcctccaaagcagccgaggaggagggagaagatccgcaggttcccacaaggtcaaggacctgctaagcccctgcctaaggattcacctcccaggaaggactcgccacacggcagggaggcggcagggccttcagggcttggagtgcccttgtgggcccccagacctcaccccttcctcatctgtcacgtgcgagtggaagaaggtgtcacttccagttctaagaaggggaacctccaccatagtgagggagggagcaggagagagattttagtcagaaagtccagctcccatgactcagtttcccttttggtttcacctcctataaactggggaccatggtccctgctgtgcccatctggtaggaagaccatcataagatgctttgaaggtgaagttgaaggtcaccaagtggtggcacgtgcaataatggtcttcagtctgaggcaatgaaaagataggggagtgggtgcagtgagtggagggcaggcccaggctggtcagggacagggtttgaccctctggctttgtcctcagatgtgaatgaatgcacctccggacaaaacccatgccacagctccacccactgcctcaacaacgtgggcagctatcagtgccgctgccgcccgggctggcaaccgattccggggtcccccaatggcccaaacaataccgtctgtgaaggtcgagagctcagatcccacgttcccagagacccacaaacatctgatcacatgttcaacggcgcccacacaaaccaagcagaatgagcgctggaggcgcccgactgtgtcaggcgttcattcttctgaggctagatgagaaaagagcaagggtcctgcggaaggagctggggtactgaggggggaggctcagggggaccccaggcaacagctgatgactcactgggaggaaggcgtttcaccatattcataacctgctcatctgcacggggcccacctgctgtgcccaggcctctccacgcttccataacccagcgtccacctctccaaggggggcactaatgccgggaggaacgagctgggggcacagacaggagacaggaccctctccaggctgggacaggacctgacccccttcttcctgtcctcagatgtggacgagtgcagctccgggcagcatcagtgtgacagctccaccgtctgcttcaacaccgtgggttcatacagctgccgctgccgcccaggctggaagcccagacacggaatcccgaataaccaaaaggacactgtctgtgaaggtatgacctggccctagaagctccccacccccagcacacacactgacacgctcccgcctaatgagccgcttgtcttgttccctacagatatgactttctccacctggaccccgccccctggagtccacagccaggtgagtggcccccacagggacgaggcggcgggaactccatccacacagcactgcatccgtctccttgttctaaacttcccacccgccgtccaggctctctgacccccacatctcctctctctgcagacgctttcccgattcttcgacaaagtccaggacctgggcagagactacaagccaggcttggccaataacaccatccaggtaaggacaggacccagggcaagggggcgaggcaggaaggtggctgcatcccacagaggcctggggcagtttgggtctgggaggggacaggacccaatgcagtgggtgctggtctgactcccagcatctttcagggctggtggaagctaatggacacccaagtgcacttaatatctttcctcttgctttccctggactttgggtttaggtcagcattgatattgcacagaagtgttggcctctgaggggccataaccagagtcaaggatcacctagggaatcccatccaatgtcattttttttttagacggagtctcactctgtcgccagtccagagtttagtggcccgatctcagctcactgcagtctctgcctcccgggttcaagcgattctcctccctcagccccctgagtagctaggattacaggcacacgccaccacgcccggctaatttttgtatttttagtagagacggggtttcaccatgttggccaggatggtctcgatctcctgaccttgtgacccaccctcatcggcctcccaaagtgctggaattagaggcgtgagccaccgcgcccagccgccaatgccatcttcatcccccagatagacagtctctaggatctgttccctggggctgagcggttggagtcttcatgcgggccctctggcccatggctcactaggtctgtgtccacatccctccagagcatcttacaggcgctggatgagctgctggaggcccctggggacctggagaccctgccccgcttacagcagcactgtgtggccagtcacctgctggatggcctagaggatgtcctcagaggcctgagcaagaacctttccaatgggctgttgaacttcagttatcctgcaggcacaggtaggtccctgggtctgccccagactccagctctgcatgttttctgtctccttcctttcccagtcccaccagagccaagtgaccacacctgtatatcagtgttactctcatcgacaaactaaaataagagattaaaaagaaatacataccagtctggacaagatggcaatactcaatctctacaaaaaaaaaaaaatcttttaaattagacagatgtgatggcacaactgtggccccagctagttgggaggctgaggtgagaggatcgcttgagcccaggaggttgagtctgcagtgaactatgattgcatcactgtactccagcctggttcacagggcaagaccccatctcaaaaaagagaagacgaagaggaggaggaggaagaagaagaagaagagaaggaggaggaggtgaagaggaggaggaggataaagaggaagaaaaaggaggaagagggcaggagggggaggacagaaggaggaggacaggaggagaggaggggaggaggaagaggaggaaagaggatgggaggaggaggacaagaggaagaggacgggaggaggaggacaggatggggaggagaggaggaggagagaaggaggaggagaggaggaagaggaggaatggagaaagagaggaggaggagaaaaggaggaggagaggaggaggaggagaggagaaggaggaggaatggaggaggagaggaggcagaggaagataggaggaggagaaggagaagaggaggagaagagaaggaggagaggaggaggagacgagaaagagcggaggagaggaggagaggaggaggagcacaggaggaggaggagaggagtagaggaagaaggaagaatagaaagaaggaaaggaaaggaaaaagaaatgcataaataacatgcattttcattgtaatatgaattatcagcagtaaataattatgatgaagaatttaaaataacaaaatagaaatcacttcaaaagagaacaatttatttaatttggaatactataaattaatatcatttgatctctaagtatgatttctttcttgtttgtttttgtttgttttgttttgttttgttttgtttttttgagacagagtctcactctgtcgcccaggctggagagcagtgacgtgatctcggctcactgcaagctccgcctcctgggttgacagcattctcctgtctcagcctcccgagtagctgggactacaggtgcccgccaccacgcccagctaattttttgtatttttagtagagacggggttgcaccgtgttagccaggatggtctctcgatctcctgaccttgtgcataatccacttgcctcggcttcccaaagtgctgggattacaggcgtgagccaccacgcccggcctgcataataatttttaaagcatgaaaatgcatatagcattaattttattaagataaaattcacataacatcaagttcaccactttaaagtgtctgtttcacgtggggcgtgcctgtaatgccaggcgctcacacctgtaatcctagcactttgggaggctgaaatgggtggatcacttgaggtcaggagttcaaatccagcctggccaacatagtgaaaccccatctctactgaaaatacaaaaattagacaggcgtggtggtgcacacctgtaatcctagctactcaagaggctgaggcaggagaatcacttgagcttgggaggcagaggttgcagtgagccaagtttgcaccactgcactccagactacgtgagacagagtgccactccatctcaaacaaaaaaattaaaattaaaaataaaaataagaaaaaattttaaaagcgtgtgtttcggtagaatctagtatattcagaaagttatacaaccgtcacctctatttctaaaatgcttgcatcagcccaaaagtgaacactgcacatggtaagcaaccactcctcattcctcccttcctccaacccatggcagccgctaatctgctttctgtttccatggattttcctaatcaggatatctcacataaatggaatcatacaatatgtgaccatctagtctagctcctttcacctagcataatggttttgaggttcatgcatgccatagcatttatcaatatttcattctttttcatggctgaatactattctattgtatgaatacatcactttttttttttttatccatccatccattgatggacatttgggttgtttccaccttttggctgttgacaaaaagagtcaaactctgtaaaatatttgaagagatttattgtgagctaaatatgaatgtatgactgaccatggcccgtgacacagccctcaggagcccctgagaacatattcccaagtgaagtggtgttgttttctggggtaatacctgaggttcattgcctcatgccaagaaaattaaggacacagacacacaccaggagtgagtttatgagtggaggtttaataggcaaaagaaagagaaaggagaacaactctctctcttgtgagagaaaggaatgcccaaatgggacttcccacccatgccagagggcaccggattttatagacaggcttgaggaggcagtgtctaatttacatagggcccaaagattggttggaacaggtgtgacgtttacataaggtgcagggaagctggctgccccaccctaatcttattatgcaaatggagtcttcgccagctccttactgtatacgtggttggcaaagagatgggaagatggagccgccattttcaacatgcctagtcccaggtggccttttcctattggcacagctgccagcattcacctgtgcaggcttccagcttgcttgcctatgtctgcagctcgattttacaggctgctctttgttagaaaaaaaaaaaatgatataggggctgattttcattaaaaggaaagacttacccaggacttccttacccacactctctgcctaaataattcctttttaacttctatatcacaaggtagtcgtggtacagcttgttgtgattgttgttgttgttgtcttctgttggagacagagtcttgctctgtcaaccaggctggagtgcagtggtgcgatcctggctcactgtagactccacctcccgggttcaagcgattctcctgcctcagcctcccaagtagctgggactacaggcgcccgccaccacgcccggctaattattttgtgtgttttcagtaaagacagggtttcgccatgttaaccaggatggtcttgatgtcctgacctcgtgatccgcctgcctcggcctcccaaagtgctgggattacaggcgtgagacaccacgcctgcccccgcaacttggttttatacattttagggggacatgagacatcaatcaaatatgtttaagctatacattggttcagtccagaaatgcaggacaatttgaagtggaggtgtggggcgggtgcttccagattataggtagatttaagcttttctgattggcagttggttgaaggagttattatccatagaaaagaatgtctggaatacgataaggggctgtaaaggctgaagttgtatcatccagatgaactcttcaggtatcaggcttcagagagaatagatggtaaatgttccttattaagcttcaggtctgtgttatgttaaatgctggtcagcttttcctcaactccaaaagggaggagggcatgacacatgtcagaccccccacttcccaccatagcctgaactagtctttccagttaaatttagagtgccctggccaaaaaggaagtccattcagatggttttgaggggggcgggagaggacttcgaattttttttttttgtttagatggctggtgtgaatagtgctcttatgaatattagtggacacattttgggttgaataactgtcttcagttctttggggtatatactaaggagtggaattgctgggtgatatggtaattctatgttcagcttttggaggaaccaccaaactttttccaaagcagctgcatcattctaattccccaccagaaacatacacgggttatcctctccacaccttcagcaaaacttgttacttccgttttcttttgttctgtttttaattgcttttattgatacataaatcttacatatttatgggtacatgtgatactttgttgcttgcctagaatgtgtaacgatcaagtcagggtatctcaggtatccaccactttgagtacttatcatttgtatgtgttgggaacaattgaagtcctcttttgtaactactttgaaatacatcatacagtcttgttaattatagtcactctgctctgctgtcaaacaatagaacttgggccgggcacggtggctcacgcctgtaatcccagcactttgggaggccgaggtgggaggatcacgaggtcaggagatcgacaccatcctggctaacagggtgaaaccccatctctactaaaaatacaaaaaattagccaggcgtggtggcgggtgcctgtagtcccagctactcgggagactgaggcaggagaatggcgtgaacctgggaggcgaagcttgcagtgagccaagatggtgccactgcactccagcctgggtgacagagtgagactccgtctcaaaaaaaaaaaaaaaaaaaaatctgcttaataagcggagaggtggctggcttggtggctcacacccgtaatcctagcactttgggaggtcaaggcaggcagatcacctgacatcaggagtttgagcccagcctggccaatatggtgaaaccctggctattaaaaatacaaaaattagctgggcttagtgacacgtgcctgtaatcccagctacctgggaagctgaggcaggagattcgctggaacccaggaggcagaggctgcagtgagctgagatcgtgccactgcactccagcctgggtgacagagtgagactccgtctaaaaaaaaaaaatatatatatatatatgtataacttatactttttgtctaactgtatgtctctacctgttaactcacctctcttcatcccagtgatacaggagtgaaaaagaaattatttaggcagttagcaagggtcagagagtcctcagtaaggtttcccttttaataaaaaccagcccccaaatcatttcttttctaataacgagcagcctgaaaattcgagctgcagacatagaaaagcaagctggaagcttgcacgggtgaatgccggcagctgtgccaataggaaaaggttacctgggggcccgacatgttcaacgtgggggctccctcttcctttatctttgtcaaccacgtgtacagtacagaagcaggcaacgtggagccagccaggtagagaatccatttgcataataaaagattaggctggggcagccagtttcttcatgctgtatgtaaatggctcacctgttcagaccaatctttaagccttatgtaaatcggacaccgcctacccaagctcatctataaaacttcgtgcatttcaccacgcaaccggaagacccacttgggagcccctgtctctctgcaggagagggagttattctctttttcctttttttttttttgagacggagtcgtgctgtgtcccgcagactgcagtgcaggggcgcgatctcggctcactgcaagctccgcctcccggggtcacgccattctcctgcctcagcctccggagtagctgggactacaggcgcccaccaccatgcccagctaatttttttgtatttttagtagagacggggtttcaccgtgttatccaggttggtctcgatctcctgacctcgtgatcctcccgcctcagcctcccaaagtgctgggattacaggcgtgagccactgcgcccagccagagctattctttttttctttcgcctattaaatctccactcttaaactaacttcttatgtgtccacatcctcaatttccctggtgtgaaacagtgaaacttgggtatttacaccacttcactagtgagatgatacctcattgtggttttgatttgcccttccctgataactaatgatgtttaacatcttttcatgtgtttgctcaccatttgcatgtcatttttggagaaatgtctattcaaatcctttgcctatttttaattgtgctgtttgtcttttggttgtccagttgtaagcattctttaaacattctgatagtagatcatctgagatgaatgatttgcaaaatattttctcccgttctgtagattgtcttttcattttcttgtcttttcactttaatgaatagaagttttaaatcttgaagacatttattttatctaatttttcttttgttgcatgtgcaattggtgtcatatttaagaattcattgacagggggtggtggctcacgcctgtaatcccagcactttggaaggccaatgtgggcggatcacctgaggtcaggagtttgagaccagcctggccaacaaggcgaaaccctgtccttactaaaaataaaaataaaaataattaaccagatgtgttggcacatgactgtaatcctagctactcaggaggctgaggcaggagaattgcttgaacctgggacacggaggttgcagtgagctgagattgtgccactacactccagcctggatgacagagcaagactctgtcccaaaaataaaaataaaaaaagaattcattaccgcatccaagaccaggaagatttacccctgtttcctcctaagtgttctagttttagcttctaagcttaggtcactggcttattttgagttaatttttgtttgttgtgtgagggtagggttaaaacttccttattttcatgttactatccagtaatcccagcactatttgttgaagagattattctttcccccattgtatagtcttggcaccccttgttgagaatcaactgaccataagatggatgggtttattcctggactctcaattctattctgttgtgtccttatgccagtattactgtcagtttgtagtaagttttggaatcaggtagtgtgaattctccaaccttcttgttcttcttcaagattgtttttgggtatttggattccttacaacaccatatgaattttaagatgaacttttcttattctgcaaagtaataataggctaaaatattgatagggattgcattgagtctctagaccactttgggggagtattgttatcttaacaatagaaagtcttccaatccatgaacataggctgtctttccatttagagagggccttttatttctttcaaacacttttgtaggccagaagcagtggctcacgcccaatattgtcagaggccaccaaggtgggagaattgcttgagcccaggagttcaagcccagccttggaaatatactgaaacatgatctccacaaaaaatttaaaaattagctggctgtggtagtgagcacctgtagtgccagctacttgggaggctgaggtggaaggattggttgagcccaggagactgcagtgagctatgatcatgccactgcactctagcctgaccaacaggtgagaatttgtctcaaaaaaaaaaaaatactcaaaaaactttcctataagtcttgagtctccttcgtaaaatgtattcctaacatttttgctgaaaatttttgcatctatgtccataagaaatattggtctataattttattttcttgtgatgtcttcatctggttttgttatcagagcaatattagactcataaaattacttaaatagtgtttcctcctcttccatatttttaaaaaggtaaaagattcatgttcattctttcttaagctattggtagaactcaccagtgaagtcatctggtctgggctgttagttttgagaggttttgattactgatttaatctgttataggtctgttcagatcttctatttcttcttgagttgatttcagtggtttgtgtgtcactaggaatgtgtccatttcatctaggttacctactttgtagatatacagttgctcacactattctcttccgatattttgtaacttcagactatatatgtatatagtatatatattaagtcatagtatatcaaccactgcaggcacgattcctaacctaaccccccttatgtaattcccacaactcatagaattgtccctggaggtgcagaagcaagtagacaggagtgtcaccttgagacagaatcaggcagtgatgcagctcgactggaatcaggcacagaaatctggtgacccaggtaatggctgagatggggaggtggtcagtgaagcttcatggtgggaaaagatgcccaagaatgtgaaaaataagaaatttaaagattaaagggaagtgtgggatgtttggagtggggctgtaagaggagagtccagctgaggaggaacggaccttgttggggaccctgagatattaaccgtggccttgtcctgcaggatgtgatacacatagaactaaagattgaaggaaagtgtgggaggggtgaagccaaaggaaggtagcccatctgagtagaaagttctgtgcagggcagaggaactctggtgtggacccagagattcaacctactccaagattcaaccttggcctcgccctgcaggcccttctgtggtgggccttgtctccattccagggatgggcaagttgctggctgaggcccctctggtcctggaacctgagaagcagatgcttctgcatgagacacaccagggcttgctgcaggacggctcccccatcctgctctcagatgtgatctctgcctttctgagcaacaacgacacccaaaacctcagctccccagttaccttcaccttctcccaccgtgtgagtgctggtggagttggttggtgggtgaatagtctgagtccgggcatagccttgctgctcagctcagccctggggctcaggggtctctgttatgggtacattatctccccaggaaagtcagtccttcccaagccggctttgggtaagcatttctgagcatccgacccaccagctcacacctgtgttcttttttttattgagacagagtctcgctgtgtcagccaggctggagtgcagtggcacgatctcagctcactgcagtctccgcctcccaggttcgagcgattctcctgcctcagcctccctaatagctgggactacaggtgtgtgctaccacgcccagctaatttttgtattttttttttttttttttttttttagtagagatggggtttcaccatgttggccaggctggtctcaaactcctgacctcaagtgatccacctgcttcagcctcccaaagtgctgggattacaggcgtgagccaccatgcccagcccacacctgtgttctgttcctgcagtcagtgatcccgagacagaaggtgctctgtgtcttctgggagcatggccagaatggatgtggtcactgggccaccacaggctgcagcacaataggcaccagagacaccagcaccatctgccgttgcacccacctgagcagctttgccgtcctcatggcccactacgatgtgcaggtgagacccttaggaggggatgcactctgcatttattgccgtgtaacaaatccccagagacgtagcagccttttaaaaaaatatatgcattatctcacagctctgggtcagcaagctggcatagcaagatggcttttctgctcagggtcttacaagactgaaatcatcatgtcaccccacccccagggctgccatttcatctgagacttgaggtccttctccaagctccctggttgctggcagaattcagtttcttgtgattgtaggactgaagtctcactttcttagctgtcaggaaggcatcacattcagttcctagaggccactcatatcccttctcacatgaccctctggcaaacttctagcattcctatctggaggacaaggaaaaaaatttcctcacgcagaatccctcttaagctttgtattagtcgaggttctctagagggacagaactaatggaatagatagatagatagatagatagatagatagatagatagatagatagataatttctgcctgcttatattctagccacgctggcagctgattagatggtgcccacccagattaagagtggatctgcctttcccaacccactgactcaaatatatacatatatagagatatatagagagagatctatatatatagatagatatagatatatataaaggggagtttattaggtattaactcacatgatcacaaggtcccacgataggccgtctgcaggctgaggagcaaggagagccagtccgagtcccagaactgaagaacttggagtctgatgttcaagggcaggaagcatccagcacaggagaaagatgtaggctgggaggctaggccagtctctcttttcacatttttctgcctgcttatattctagccacgctggcagctgattagacagtgcccacccagattaagagtggatctgcctttcccagtccactgactcaaatgttaatttcctttggcaacaccctgatagacacacccaggatcaatactttgtatccttcaatccaatcaagttgacacccagtattaaccgtcacaagttttgaatctgattctccaggaagagcccagagcttataaagactcatctgattaggtctggcccacccagggtaaactctgccttttttttttttttagacagggtctctgtcacccaggctggagtgcagtggcacagtcatagctcactgcagccttgaccttctgggttcaagggatcctcctgcctcagcctcctgagtagctgggactacaggcatgcaccaccatgcccggctaatttttgtatttttactagagatggggtttcaccaagttggccaggctggtctcgaactccttacctcaagtaatccacccgcctcggcctcccaaagtgctggtattacaggcaggaaccaccacacctcgcccacctgactttcttatctcatcacttagagtagatccaagggacatcatcatcatatcctgcgggagaaagggccaaaccacccttttgttttccatgacaggaacatcttatcaatatcctcccaggcagcaagccataccgcccagcccctcccgcccagacctgtaattaccccagcctgtaagcggcagtgggttctggcacgaagctagctcccccctccacaagtctcctgctggacataaacctgcattgctgtagagctgccaactctctgtctttctttaactctcactttcccttcaaaacctaacgggcccacacctggaatctctgtgctttgggaggctgaaacgggaggatcacttgagcccaggaattcgagaccagcctgtgcaacacagaaagaccctgtctctacaaaaataaaaaaaaaattaggacgggtgtggtggctcacgactgtaatcccagcactttgggaggccgaggcgggtggatcatgaggtcaggagttcaagaccagcctggccaacatggtgtgaccctgtctctactaaaagtataaaaattagcctggcatggaggcacgtgcctgtaatcccaactacttgggaggctgagacacgagaatcgcttgaaccgaggagacagaggctgcagtgagcagagatcgcaccactgcactccagcctgggcgacagagcaagactgtcaaaaaaaaaaattagcagggcatggtgccatacatgactataatgcacacccgttgtcacctacatgactacgcctgtattcctagccacccatgaggctcgggtgacaccttcgcaaattaaattaaattggtgacatcttcacaaatttacctggctccctcattcttgaatgtgctatgccaaagtaaaaattttaaatggtggtggggtcattaccccagagcaggaaagcatcactgtgaactcttcttttccgggtcccaggaggaggatcccgtgctgactgtcatcacctacatggggctgagcgtctctctgctgtgcctcctcctggcggccctcacttttctcctgtgtaaagccatccagaacaccagcacctcactgcatctgcagctctcgctctgcctcttcctggcccacctcctcttcctcgtggcaattgatcaaaccggacacaaggtattgacagcggtgttccagagggctcccttccttgacgcaggacacgctggctcatggaatgatgcggcatttgattccttaatataatagcccggctgggcgtggtggctcatgcctgtaatcccagcactttgggagactgaggtgggcggatcacctcaggtcgggagtttgagaccagcctgaccaacatggagaaaccccgtctctactaaaaatacaaaattagccaggcatggtggcgcatacctgtaatcccagttactcaggaggctgaggcaagagaatctcttgaacccaggaggtggaggttgtggtaagccgagatcgcaccattgcatgccagcctgggcaacaagagtgaaattccgtctcaaaaaaaaaaaaaaaaaaatatatatatatatatatatgtgtgtgtgtgtgtgtgtgtatacgtatatatacacgtatatatatataggcccttgagtcctggggaatattagaagagaacaccaccaatttcccattgcctgactcaactacacagcaaatattctctataatgccatcaaatatctcagaattctccccaacaacatggtacaggagaagccatgggccacactcaaatctgatcgccgctctgggcgtcttttacttctccgccaggtgctgtgctccatcatcgccggtaccttgcactatctctacctggccaccttgacctggatgctgctggaggccctgtacctcttcctcactgcacggaacctgacggtggtcaactactcaagcatcaacagattcatgaagaagctcatgttccctgtgggctacggagtcccagctgtgacagtggccatttctgcagcctccaggcctcacctttatggaacaccttcccggttagtgcaaattctcacaatccttatcttctccggcataaccatggccattgctagaacctagatagcatttttatgacacctgcttttaattagatcagtagggattttaggctctattggttggattacactgcagctccttattcttagctattgagtttatttcaattacatttcaagctaggcattgtagaagagggttttcggaaattttctgaatgaaataaagggcaagcctttggaaagcacgaataggacaaagacactatgtcataatccctagggcatctttctgctagaagattataataattaaggagggtcaggctcagtggctcgcacctggaatccctgtgctttgggaggctgacacaggaggatcacttgagcccaggatcttgagaccagcctgggcaacacagcaagaccccttctttacaaaatttaaaaaattagcagggcctactggcacacacctgtagtcctagctactcaggaggctgaggtgggaggatcgcttgagcccaggagttcgaagttgcagtgagctatgattgcaccactgcactccagcctgagcaagagagaaagaccttgtctctaaaaaataataataatagttagggataatgcatttaaatctgtctcactgaaatagtaagaatgaaatcaaagtgctcaagacagcattttttaaaaatctaacctactttatctaaatcttttaggatactccaatttttcatttgaaaaagtagacctgatttgaataactaaaataattgatattgtcatagttaagatttatgagatgcttactttgtgccaggtacttttctagctagtttgtttgtttgtttgtttgccaattgcactggttgaaacctccaggacactgttgactagaacagcagacaattttgtcttgctcctgtacttagggagaaagcattgactctgtcctcattaatgtatgatgttagctgaggatttttcatagataccctttttcaggttaaagaaattcttttctattcctcatgtgttcggtattttgatgaagaaagagtgtttgattttgtcaaatgcttttccgaagcatataatttttaattatatggggtaaaaattatatgatcacataattagataatcatataattatataattatattagattattttatatgtaatttttatataaatatatatctatgtaatttatatattaatattatataactataaataattatataacatagctataaataattatgtaactatgaataattacataacatagctataaataattatgtaactatgaataattacataacatagctataaataattatgtaactatgaataattacataacatagctataaataattatgtaactatgaataattacataacatagctataaataattatgtaactatgaataattacataacatagctataaataattatgtaactatgaataattacataacatagctataaataattatgtaactatgaataattacataacatagctataaataattatgtaactatgaatacataacatagctataaataattatgtaactatgaataattacataacacagctataaataattatgtaactatgaataattacataacatagctataaataattatgtaactatgaataattacataacatagctataaataattatgtaactataaataattacataacatagctataaataattatataactataaataattacataacatagctataaataattatgtaactataaataattatataacatagctataaataattatataactataaataattacataacataactataaataattatataaccataaataattatattacataactataaataattatattacataactaaataattatattacataactaattatattacataactataaataattatattacataactataaataattatataactataaataattatattatataactataaataatatgatcacattaaattgtataatcatatattatcatatcattataattatattatatatgtatatatcatatataattacatataattatataagcataattatatgcttccttaattctttattttattaatacacatattaatagaataaaggattaaaattatatgatcacattataattatatggttatataattatatggttatatatgattaattatttacctttgagtcctggggaatattatatggttatatatggctaattatatggttatataattataatgtgatcttataattatatgattacataattatataactataatgtgatcatataactttaatcctttattctattaatatgtgtattacattgatggatgattagatgttaaactagccttacattcctgggataattaccacctggtcagggtatataatccttttaatatgttgcaggacttggtcggctagtatttttttgaggatttttgcattcattttcataagggattttggtctgcagttttcttttcttgtgatgtctatttctgggtttggtgtcagagtaacactggcctcgtagaagatttggaaagtgcttcctctttttccgtttttggttttgtgaactattaacactaatcctccaaaagttaaccatagagctaccatatgacccaggtatatacccaaaacaaatgaaaacgatgtccacaaaaaaacttgtacatgatgcagccataaagaataaggagatcggccgggtgcggtggctcacacctggaatcccagcactttgggaggccgaggcgagcagatcacaaggtcaagagattgagaccatcctggccaacatggtgaaaccccgtctctactaaaaatacaaaagttagctgggcgtggtcgtgggtgcctgtaatcccagctacttgggaggctgaggcacaagaatcgcttgaacctgggaggtggagtttgcagtgagccaagatggctccattgcacttcagcctggccacagagcgagactccatctcaaaaaaataaaataacaagatcatgtcctttgcagcaatatggatggagctggaggccattattctaagtgaattaatgcaggaacagaaaatcaaataccgcatgttctcacttataagtaggagctaaacattgggtacccatggacacaaacaaggaaacttcagacaccgaagcctgcttgagggtggagagtgggaggagggcgaggataaataaacgacctatagagtactgtgctgatgaccagggtgacgaaataatctgtacaccaaaccgccgcaacatgcattttacctacataacaaacctacaaaatgtacctctgaacctaaaataaacgtttagaaaaatgttgtacatgaatgctcatagaagcagtatttagaatagtcaaaaattggaaacaatgcctatcactgatgaatgaataaacaaaacgtaatatatccacaatgaaatgttattcagccataaaaaggaacgttaagagaccattcagtcattatttatttatttagaaacactctgtcacccaggctggagtgcagtggtgccatcttggctcactgcaacctctgctctctgcttcctgggttcaagcaattctcatgcctcagcctcccgagtagctgagattacaggcatgcgccaccacacgcagctcattttttgtgtgtttttagtagagaaaggatttcaccacactgggcaagctggtctcaaactcctgatatcaaatgatccacccgcctcggctcccaaagtgctgggattacaggtgtgaaccactgcacctggcctcattcagccattttaaaatgctgcaatataggtcttgctctgttgcccaggctggagtgcagtagtgcaatcacagctcactgcagtctcgacctcctggactcaagcgatcctcccacctcagtctcccaagtaactgggaatacaggtgtgagccaccacacccggctatttttttattttttgtagaaatggggtcttgctatgttgctcaggctggtctcaagctcttggtctcaagcaatcctcccacctcggcctcccaaagtcctggtgtgagccactgtgcctggcacaaccttccatttttgtttcattgtttgaatgtgtctgattgtgtgtatcttccttgagttgaaattttggggatctaagaccgtgttcattttctccaccactgtatcttcagagccaaccacagaccctatcactaaagaaacacttaatattgttgacgtaggaaggaagtgaggaagtggtgaggagggatggaatagatttacataactgttccatatctccaaaaactgatgcattttttttaatctctcttttttcagctgctggctccaaccagaaaagggatttatatggggcttccttggacctgtctgcgccatcttctctgtgagtgacatcatctgagcatcctcgctgccccgctgagggtcattagaaaaacaagataataaagatggtgacaagagcaggaatgtcccctgggttgtcatgggttgggttaggtttcctaagaccactcttgaatcttcaaactcaacactgttactcaatatttaagaaaaagacttacgtgtataattcatattagacaggctaggatgaagagataatgggcttcaggcccggtgtggtggctcacgcctgtaatcccagcactttgggaggccgaggcaggcagatcacttgaggtcaggagttcaagaccagcctggccaacatggtgaaaccctgtctctactaaaaatacaaaaattagccaggcgtggtggtgggtgcctgtactcgggaggctgagacaggagaatcgctcgaacccgggaggcagaggtggcagtgagctgagattgcaccgccacactccagcctgggcaacagagcaagaccctgtctgaaaaaaaaaaaaaaagagataacaggtttcaaaatctattcaacaaatcttctgtggttttctgaaggtgaatttagttctctttctggtgactctctggattttgaaaaacagactctcctccctcaatagtgaagtgtccaccctccggaacacaaggtaagatggagaagggggtgataaccacacagacatgtatggttcccaagagccaagccatgtgcctcagcagccactgaggtcccttctcagggtcttttgaagggacccagaaaaaacagacgcagccatccttctatggcagccatcacagattcagtggtatcctgttgagaccccatgcaacatgttttgtccatgtctaatctcagtttgtgaatcccagagcaacaggaagaatcttcattgactggtaaatggtatatatacaaaataggatacgattcgatcacaaaacagaatgaaaccatgtcatttgcagcaacatggatgaaactggaggtcactctcttaagtgaaataagccaggcacaaaaagacaaataccacaggttgtcacttacatagagaggagccgaagaagctgatcttgtggaggcagaaagtagaatgataaaagttagccgggcatggtggcaggcacctgtagtcccagctactcgggaggctgaggcaggagaaccgcttgaacccaggaggcggaggttgcggtgagccgagatcacaccactgcactccagcctgggcaatagagtgagactctgcctcaaaaactaaaaataaaaataaagtagaatgatagattccagagtctggaaagggtatgtgagtgggcaaggcgatgaagagaggttggttaatgggtacaacatacagtcagatagaaggaataagttataatattggatggcagagtagggtgactctagttcacaatatgttgtatatttcaaaatagctagaggggagaatttgaagtgttcccaacacatacaaatggtagacacgcgaagtgacggataccccaaatactctgacttaatcatgacacactctatgcatgttaacaaaatatcacatatgtccccacaaagatgtacaaacattacgaatcaataaactaaaaggttaaaaacttctttggacttttgtcagcagtcctataacaaaagcacaaagaaggccgggcatggtggctcatgcctgtaatcccagcactttgggaagctgaggggcgggggcagatcacaaggtcaggagttcgagaccagcctggccaaagagaccagcctgaccaacgtggtgaaaccccgtctctactaaaaatacaaaaattagctgggcgtggtggcgggtgcctgtaatcccagctactccagaggctgaggcaggagaattgtttgaacctgagaggcagaggttgcagtgagccgagattgcgccactgcactccagcctaggtgacagagcaagactccaactcaaaaaaaaaagtgcaaagaacacgtacattttcttgcctgttcccacagcccagtctgcaccttcataccgtaccctcagttcaggctcctcatcacttctcatgcaatcctccctctgggaaaatcctaatcaacattaaatcctttacgggggccaggcatggtggctcacgcctgtaatcctaacactttgggaggccgaggtaggcggatcacttgaggtcatgagttcaagaccagcctggccaacatggtgaaaccccatctctactaaaaatacaaaaattagccaggcatggtggtgggcgtctataatcccagctactcaggaggctgaggtgggagagtcacttcacctgggaggcagaggttgcagtgagccaagatcacgccactgcactccagcctgggtgacagagggagattccatctcaaaaaaataaataaataaaaataaaaataattcctctacagggtttgagcagtttgttgttccaggcacccttgtgcaaaattaatgaactttcttctagattcctatcattatcctttccaaattttgtgtgtcatgcctggttgtattggtgatttgaagagactgtgtagttcatgaacacagaaatctctttgcatattcaaaaataatcctttttttttttttgagacagagtctcgctctgtcactcccaggctggagtgcagtggctctatctcgactcactgcaacctccgcctcctgagttcaagcaattctactgccccagcctcctgagtagctgggattacaggcgcccccccgaccacacctggctaatttttgtatttttttagtagagaccaggtttcaccatgttggtcgggctggtctcgaactcctgacctcaggtgatctgcctgccttggccccccaaagtgctgggattacaggcatgagccaccgtggccaaccccaggtggcatttctactgtgggctatagttgacagtaaatatctaagctgaccacagcccagtctgagaaatcatctacaggggagggcaggggactccatgggcacagatgaaatagccttgattctctttctctgggtgcatcaccagcctcctttcttactcaatccaggatgctggcatttaaagcgacagctcagctgttcatcctgggctgcacgtggtgtctgggcatcttgcaggtgggtccggctgcccgggtcatggcctacctcttcaccatcatcaacagcctgcagggtgtcttcatcttcctggtgtactgcctcctcagccagcaggtaccactgcccagctcccacccaggactcttcctgtcctcactgctctctgtgagctgacccagtacacactttgcctctgcaggtccgggagcaatatgggaaatggtccaaagggatcaggaaattgaaaactgagtctgagatgcacacactctccagcagtgctaaggctgacacctccaaacccagcacggtaagatcacgcattgctccagagcacttcactaaccgacccacctgaggagcatgtgcctatcacacaaggaaacctgggaatacagcaggcaatgccctagaaaggctcgcatctgagtacgccttgactcattaaccattagcaatgatctcagtttaaatgtttttttttaatcagtcatagcctgtcatcccagcatcactgtcatcccagcatttgggaggcctaggcaagaggatcacctgaggccaggagtgcaagatgaccctgggcaacatagcaagatcccatctctacaaaaaataaaaataaaaattagccagacatggtggcatacacctgtagtcccagctactcctcgggaggctgaggtgggacaatcacttgagcccagtaggttgaggctgtagtgagccatgatcatgccactgcactccagcccaggcaacagagtgagaccctatcccaaaaattaaaaaaaaaaaaaaaatccttggctcttactccttagggaactgtgcttagtttgactctcacacacaaacactcctattggacacctactatgtgccatgcatggttaactaaatgagcaggaattgatctgaagcagtgatgcacattctacctagaagcatttaccctatccctttcctctacatacaagtctatctcacagtcacttggcaccaccacccaataaggcaagcatgggaggaaatgctgtacatatttatgaattaaacagaaataagtgttaactgggatttaggctttcttaacaagtctgcctctaccaactaccacttagtagtgggtagtgggcagtgcctctactcagcaccctctacccactgcaaaactcagaaagaaaagcagtaatggggccagcgatggtggctcatgcctgtaatcccagcactttaggaggctgaggcaggacaactgcttgaggccaagagttcaagactagcctgagcaacccagtgacacctcgtccctataaagcatttaaaaattagctgagggtggttgtgtgcatctgtagtcccagcaactcaggaagctgaggtgagaggatcacttgagcccaggagtttgaggctgcagtgagctatgattgtgccactgcatttcagcctggatgacagcaagacctcttctctagaaaataatacaaataacaaaattaaataaataaagttttaaaaacacactgggcaacctgggaatcagcgggtgatgccctaagagggttcacatttgagcgactccttgactcattaaccattagcaatgatctcagtttaaatgcttttttataaatcagccaggaacagtggttcaagcttgtaatgccagcactctgggaggccaaagcaggaagatcacttgagcccgggacaacttggggatcacttgaagccatattatcctatgcctcaatggcatgggataattcatgacattgatgactaataaccaacatttattaatgtagacatacaaagttctgttgtatttttctcacctcttcttccatcttctccagattatatgtgtaattctaaccccatttcactgatgagcaaagtaagactacttggatttgtgtcaaatgtacagagtctcggccaggcacagtgactcatgcctgtaatcccagcactttgggaggccgaggcaggtggatcacaaagtcaggagatcgagaccatcctggctaacacggtgaaaccccatctctactaaaaatacaaaaaaaaaaaaaaaatagccaggcgtggtggtgggcgactgtagtcccagctactcgggaggctgtggcaggagaatggtgtgaacccaggaggcagagctggcagtgagccgagatcgcgccaatgcactccagcctgggcaacagaacgagactctgtctcaaaaaaaaaaaaaaaaaaaaaagaaatgtacagagtctcttcagccagtgagatttaggcctcttgagtcttgcttcacatttaaagagcccattcagggccaggcaccgtggctcatacctataatcttagcacttcaggaggccaaggcaagcaaatcacctgaggccaggagttcgagaccagcctggccaacatggcaaaactccatctttactaaaaatacataaattagccaggcatggtggtgcacacctataatcccagctactcaggaggctgaggcacaagaattgcttgagccggggaggtaaaagttgtggtgagccaagattgccccatgccactgtactccaccctgggcaacacagcaatactctgccaaaaaaaaaaaaaaaaaaaacagcctattcagcctgggcaatgtaacgagaccccatctctataaaaaagttttaaaaattcgctgggcgtggtggcacatgcctgtagtcccagctacttgggaggcgagaggatcgcttgagcccaggagttggagaccaacctgtgcaacatggcaaaatcccatctctacacaaaatataaaaactagctgggcatggtggtgcatgcctataatcccagctgcttgggaggctgaggcaggaggatcacttgagcccaggagttcaaggccacggtgagctacgattatgccactgcactacagcctgggtgacagagcaagaccccttgtgtaaagtaaatacataaataataaattttttaaagtcttgaaaagaagcccaccatgctgccaggcactttttccacatttgcatttctctcattacctttttccttttttatttttcttttttttttttttttgagacggagtttcgctcttattacccaggctggagtacaatggtgctatctcagctcaccgcaacctccgcctcctgggttaaagcgattctcctgcctccacctcccaagtagctgggattacaggcatgtgccaccaagcctggctaattttgtatttttactagagacgggatttctccatgttggtcaggttggtctcaaattcccgacctcaggtgatctgcccgcctcagcctcccaaagtactaggattacaagcgtgagccaccacacctggcctcattacctttttcatccaacctgcacaatccctgtttccaacacaggatcttctatttccttttctgttctcactaaatgccctcaccacctatgtcacacacacacacacacacaaatccaagtcatcatgatgacacattgtaaactccatgctctcaatcatacaaccaaacatgaccatccaccttccacattcccatattattgactgtccatcctctttttttttttttagacagagtctcactcgctctgtcacccaggctggagtgcagtgacaccatctgggctcactgcaaactctgcctccagggttcaagtgattctcgtgcctcagcctcccgagtagctaggattacaggcacccgccaccacgcctggctaatttttgtatttttagtagagacagggtttcaccatgttggccaggctagtcttgaactcctggcctcaagccatctgcccgccttggcctcccaaagtgctgggattacagacgtgagccactgcgcccagtccatccctgtctttgactctcctacagccttcatactttataaagaccttgatgattacattgggttcaccaagacaatccagggcaattttcccatctcaagatccttttcataaccgtatctacaaagtctctttctccatgtcaggtagcatttacaggttctggggattagggatgtgaacctctttggggggtcattattctgtctctcatacccatatagtttgggtttacctattgtagaatcatcaaaacctgcctcatattgtagatatttgcataaatatttatcaccctttcaccccacacagccactagaaatgatatatacacattgaagaaaggaaacatgtacttttgtgtagggaattagagttaggtcatcagatagccaacaaaggcttctgaaatttaaggtgtgtgatcagatatttctaccagctactgacaaaggaccaagaaaatccattgcattacttaatgtcattttatcttctactgtttcctaaaacttgttatctctggtgtttgaaaatgtatgtgccttggccggacacgctggctcacacctgtaatcccaacactttgggaggccaaggcgggcagagcacttgaggtcaggagttcgagaccagcctggccaacatggcaagaccccacctctactaaaaacacaaaaactagccaggcatggtggtggatgcttgtaattccagctacttgggaggctgaggcacaagaatcactcgaacccaggaggcagaggttgcagtaaagccaagatcgagccactgcactccagcctgggtgacagagtgagactatgtctcaaaaaaaaaaaaaaaaagaaaagaaaagaaaagaaaatgtgtgtcttgctcttattctgcagtctctgccatggctttctgcagtcttgtttcataaaaacaaaatgtcttgtgatcactactgaataatctcctgaagcacaatatttattaatctcttgagcacatattaattatgcattaaatactaactatataagcaagtgaagctgaccatctcgaaccatagaaattacactgagtttcctatgaacctaggaataaataactcatattggagatggaaaagatatggaaaaaaggaattcaggctgggcgcagtggctcacgcttgtaatcccagcactttgggaggccgaggcaggcagatcactttagctcaggacttcaagaccagcctgggtaacatggcaaaattccgtttctacaaaaagtataaaaattagccaggcatggtggtgcatgcctgtagtcccagctaccgggaggctaagattggagtattgcttgagcccaggaggtcaagtatccagtgagctatgatcccatcactgcactccagcctgggcaacaaagcaagacccctgtctcaggggaaaaaaaaggaattcagtgctttttcctttaaatcttatgtggttaacaatagataacacttctaacatttcattaatttcccacctggcttcttttatgactccacatttaaagcaacattacacaaaccctggagctccgaggtcaagacatacatttggaaacagatagatctttttaaatgttttttaattgatccataatagacgtacatagtttctaggtacatgtgatcatttgctacactcatataagcaaatcagggtaactaagatatctatcaccttaaatacttatcttttctttgtactaataacattcaaattattctcttctagctagtttgaaatgtatggtagattaatgttaactataatcaccctacttcagatacaacttttttttttttttttgagagggagttttactctgtcgcccagactggagtacagtggcaccatcttggctcactgtgacccccacctcccagattcaagcgattctcctgcctcagcctcccgagtagctgggactacaggcgcctgccaccaagcccagctaattgattattattattactactgtattctagtagagacagggtttcaccatgttggccaggctggtctcaaactcctgacctcaagtgatctgcccgcctcggcctcccaaagtgctgggattacaggtgtgagtcaccacacctggcccagatagatactttactgcaacacatttacttcctcttaactttggtaacttggggtttaggaggcatacaaagatggggcttctgagaaagaacaggatggagcaaaatgaaagctgatatggccttttgatgttgccctgacaaatacatatggtaagaaaaccttatgattaaagacttcttggccaggcatggtggctcacacctagaattccagcatttttgggaggttgagatgggaggatcacttgagcccagaagttcaacagcagcttgggctatacagtgagaccccatctctacaaaaaattaaaattaaaaaatgagacaggagtggtggcacatgcctgtagttgcagctactcgggaggctgaggcaggaggatcacttgaacctgggagttggaggttgcagtgagcagagatggtgccactgcactccagactgggtgacagagcaaggccctgtctctaaaaagaaaaaaaaaaaaaagacttcttaaatttcttttttttaataaattttattgtgtatacacacaattgaggtttaacccatgaagttatgggatacatatagataataaaatggttatctatatgtgaagcaaattaatatatctatcatctcacatagttacttatttttgtggcacaagcagctataatctacttatttaactaatttcttatttctttttcaaaatggaaattataggttttctttctatagtcatatatatttctaaatatatatatatttctaaatatatagatattaaatatatatatttctaaatatatagatattaaatatctatatatttctaaatatatagatattaaatatctatatatttctaaatatctatatatttctaaatatctatacttctaaatatctatatttctaaatatctatatatttctaaatatctatatttctaaatatctatatatttctaaatatctatatatttctaaatatctatatatttctaaatatatatatttagaaatatctatgtatttctaaatatctatatatttctaaatatctatatacttctaaatatatctatgtttctaaatatatttagaaatatatatatatttcaggccagctcagtggctcacacctgtaatcccagcactttgggaggctgaggcaggcagatcacctgaggtcaggagttcaaggccagcctggcctacgtggtgaaaccctgtctctactaaaaatacaaaattagccgggtgtggtggcacatgcctctaatcctggctactctggaggctgagacaggagaattgcttgaacctgggaggcgggggttacagtgagtcgaggctgcgccattgcactccagcccaggcaacaagagtgaaactctgtctcaacaacaaaatacatacatatatatatatttcaggttaactagaaaaatcttctgaataagatcttccctctttgcccgtggaaaatctgaacaatctttgagccatctagaggggaaagaaaagactttgttctgtgtgtttcaagaaattcaccatgtcagcaatatgaaggatgttatggaaggcgtgctaggcattcaattcctgcagaaaccggaaatcttccatgccctgcaatgtgctcatcaaactctcagcatatggacggccagctgtggcccatatcttggtcactctgaagcacaatatttatgaagctatagaacgttaagacctctttcacagcctctccttcctacaaagactcctccaaatcttaaaatgaagcaggaaaacgagcctaagaggactttcataccgacaacatctgaaaggactagaatgttcacaccacgatctggatttcttaattttttgtttttgtttttgttgttctctagttctacgggtttgattatttagtcatgtgaaaaatattgattactcacacatagatcaagagagacacggctcctgccttcatggagcttttaggggaaaatgaagtggctcttgcagctagagttgactcagaagccgaaattcctagaaatcaggtttctactgctaggcaattgaagtataaactattttataaacactgtcttctttcgtcttcacaccaacatgcagaaaagtttctaatctcagatcggggatgtgcaacaaattccatttcaaaggaatgacctgcaaaactcctaaatattccaagcaaatgcccttaaccctgtctgttatctgctttccttgaacagaaattctacatgaccataaaacctcgaagatgggtatggcacagttcatgccctgtaatcctagcactttgggagggtgaggcaggaggatggctcaagcccaggagtttgagaccagtgtgggcaacagagtgagaaccatctctacccaaaaaaaaaattaaaaattagccaagcatggtgatgatataggagttaaggagaaatcatttaggcaaatagcaagggtaggaagtcctcagtaaggttttccatttaatgaaaagcagcccccaaaatcattttcttttctaacaaagaacagcctgtaaaatcgagctgcagacatagacaagcaagctggaagcttccacgggtgaatgccggcagctgtgccaataggaaaaagctacctagactaggcatgtccaaaatggcggctccaagttcccttctctttgccagccatgtgtacagtaaaaagcaggcaacatagtgtcagccaaagctcatttgcataataagattagggtggggtggccagctcacataggggtaggccctaggtaaatcagacaccgccttctcaagcctgtctataaaatctggtacactatgacgagggtcagatttcccattcagacgcccctctcccatgcaagagaaagagctgttctcctttctctttcttttgcctattaaacctctgctcctggccaggcacagtggctcacgcctataatcccagcactttgggaggctgaggtggtcagatcacctaaggtcaggagttcaagaccagcctggtcaacatggtgaaatcttgtctctagtaaaaatacaaaaatatatgaaatctcacatagatgataatattaagttccaaaagcaactcaacctggtagattctaattttttttgaggcagggtcttgctttgtcacccatgctggagtacaatggcacaaacactgctcactgcagcctcgacctcccaaggcctaagcaatcctcctgcctcagtccccctccaggtatttgaaactacaggtgtgtaccaccacaccgggctaatttttgtattttttgtagagacgtgggtctcactatgctgcccaggctcaggtcttaatctcctgagctcaggcaatccgcaggcctcagcctccctaagtgcggggattacaggcttgagccactgcacctagcctctatttgttttacaaaagagaaattgagatcctgaatgttaagtgacttgcctgaggccatcccactaacaggagccagggttaggattcaaaccccatccaactggtcccagagctggagcttcttgcactgccctacactacctaccatctccatcctctgggcacctttttataagaaccaaaacattacagagcattgctttgtcaactcagctgggaacatttcccagtgcaactcacatttttcactgctctgtgcctgtccgtataagctcaatgagtattgatttaggggctttggagaactttgaatgctaccccccaagtaaccattgttggcaacctggtacctctacttttagccatttctccttctctataaatagtgcagaagtaacccacttggtaacaggcatccttgccaagcctccaccactaggtcagtgtaagaattaaagaaagaggaaagaaacacaaaaagtggcttgatggttaagacaggtttattttagagaaaacacacctgagaggggctgctggctgaattaggttagagtcttttctacagactaagagtgtttaaggatttagggtgggagagtttcttagaggcttggactgcttctgtgttttttttgttgtgcttatatgggagggagagtggtgtgtttgcttttatacatttttctgcagctgtaggcataccccccaagtctgcttttagcttccctattttagtgcacctggagggaaaggaatgtgcttattaaggcccactgttttactggggcccattgtatgagggtgaagtttggcagttacccaagagacttttcctccaccttcctctgtgcccgagctgttttatctgcattttactgtctgctttttttggctgcttatagtttttaaaaaagtaatttccttaaatccagaaggctaaaaatgaagctgaaacttaaagtggcggtgtttgtccaaaataacggggctcctgctctgccagtcagtaccctcaagtcactcctgatcctcaacctccatgcctaaggctggttcaagagaccacataatatctgccttttattacatacatgatgggtgcatgggattctgcgtgccctttgcttgatatagactgctaaggtgagatggggaatatcagagtcagctgctgcttgaggaagcagaacacacagctggaggcttggaacatgtgggtccctatgagtgtagagcccatatccccatagagtctacctagagcaggggtcgccaaatgttttcttaaagagcctgatagtgtatatgttaggctttgtgagccaggtatttacagcaactcaattctaccactgtggtatgaaaacagctatagacaatcataaatgaatgatcatggctatgttttaataaaactttacagacactgaacttgaacttccattgtgatatgaaaacagctatagacaatcataaatgaatgatcatggctatgttttaataaaactttatggacactgagcttgaacttcatatatcagtcatgtgacacaaaatatcattcttctttttatttgttttcacccatttgaaaaatgtaaaaactattcttagctgtacagaaacagatggtgggtcagatttgactcactgcccatagtttccagaccatgatgttcaggttcatggaagcacttcactctacactaatttattcatccattcatcacatatgtgatgagcacatacagacacacctcagagatattgcaggctccactccagatcaccacaataatgtgaatattgcaataaagcaagtcatacacattttttggtttcccagtgtgcataaaagtatgcttaccctatactgtaatgtgtgcaatagcattatgtcttttttttaatgtacatactttaatttaaaaatagtttattcctaaaaaatgctcaagcagggacacaaagtgagcacatgctgttgggaaaatggcacctacagacttgctcgacacagggctgccataaacgttcaatttgtaaaaaatgcaatatctgtgaagggcaatgaaatgaggtattcctatactctcgtgcaaggcataatgctaaattgagagaaactaaaacatataagacagacatgatcctacatccaggaatattgatgatttgtaagatggaaaatgaggaggaaggaaaggtgcagtgcaatggactctgtggtctgggggacacacagcccctttaaggacatggcagaagtgaaacacagacgaggttggggaaggaaggtcagaggaaactctagggtcagctgatggcagcatccatttgcccccatatcttcactttttaaatttattaagtccattctctttatttatgatggaaataatgttaatttgctcatgggttgtgtttgaatattaagtaagaaaataaagattaagctctggtatgtagtaagaaacccaaagataaacaagagttaaggggcaagtaggcagcagagtttcccatctcatttctccagaattatatcatccagtttgaaacatgaaatacggctttaaggtagagtttggcttctcagctggattttaagtacaagaccacgcccaccaaagacagcagacaaaggagctagggtacacgtttgaagaaccctctccctccagctctccaccgtgggggtccactgtgagcagtttccctggctgttttccccctttctgcatctaacagtcattgcacggctggggaaacaaagacccacacaggccttagtctccttgcggggaacctacaatccggaagccagaagtatgtgactaagctcccggaagccggaagttaaggagcccggatgtcacggaactggaagtttgaaagtccaaggccactgacctgcaagcttcagaatctgcagatttggagtcttcaagccccagtagtgatcctttgatatccagaggttggattctagcgatctgaacatgcaagagagtactggatgtggaaattcttccaaattagttgggtggctgaaaagggaattataaggaggaagaatgagggaaagagaagagaaattgaatgaataaaggggccaatccttggggggagggggcaggaggggcggtgcttgagaccagggcagcatgtaagggactgcctatgaatgtcctaaatatctgctatttagggcattctgggatgggagtttgaaagactgatggtggtgtgttttgcagccacatgaatgcagctggaaccatcatcctaagtgaattaacttagaaaaccaaataccgcatgttctcacctgtaagagagagctaaatattgggtacacgtgaacataaagatgggagcattagacaatggggactactagatggaggagggagggaggagggcaagggttgaaaaactacctatcaggtactatgctcactaactggattcatttgtaccccaaacctcagcatcatgcaatatactcttgtaacaaacctaaacgtgtaccctcagaatctaaaaataaaagttgaaattaaaaaaatcactgttgatagacatttgggttgttcccagtttgggttattacaaactgctttgctagctttaactttttttaaataataaactcttaaatttacagtgcaagaaatacagaaagagagaggaagacaaagaaaaagaaaggagatagagaaaggaaaagcaaagaaagaaaaagaaaaaagtgaaagaaagagaggaagggaggaaggaaagaaggaaggaaggacagactgatggtgggataagagagaaggggctgggaagaactaagtggaacattctgaaaatgaagacagtggccgggcacggtggttcacacctgtaatcccagcactttgggagactgaggcaggcagatcacctgaggtcaggagttcaagaccagcctgaccaacatggtgaaaccccatctctactaaaaatacaaaaattaaccaggcatggtcacgggcacctgtaatcccagctactcaggaggctgcgaggcagaagaatcacttgaacctgggaggcagaggttgcagtgagcagagattgtgccattgtactccagcctgggtgacagagcgagactccatctcaaaaaataaaaaataaaaataactctcttctctctattttccttcaataaattctctttttggctaaagtagtcattttagccaaaatctttctctcaagacaacaaagaactgaggatttctgccctttccagtatacaactagcagggatcccactggcatttttctttggacagataaaattggatgtatttatcaagagcaacatgatgtttaatggatgtatacattggggaataaccagatctagcaaattaatgtatgcctcgtctcatatagttattatttggtggtgagaacaatttacatccactctcttagcattttttaagaatataatttattattaactatagtcaccatgctctacaattgatctcctgaacttcttcctcctgtctaactgaaatttgtatcccaccgggcgcagtggttcacaccataatcccagcactttgggaggctggggtgggcagatcgcttcagcccaagagttcaagcccagcctgggcaaaatggagaaaccctaactctacaaaaaatattagccaggcatggtggtgcatgcctgtggtcccagctactctggaggctgaggaggaaggatcacttgagcccagtagtttgaggctgcagtgagctgtgaccacaccactgcactccaacctgaacaacaattagaccctgtctcaaaaaacaaaaaaaaaaagtgtatcgtttgaccaacatctccccaacccaagctactggcatttagttaatagtccagggatgctgctcaacagtcttcagtgcatagggcagttcctacagcaaagaattatctggccaataaggtcacagttgagaaaccctgactgtaggatgcaatgaatctctcggtttctcttcaaaggttcagtctgttaacttccttgtctctatgcctccctgccccaagttactataaacagccttcccgtcggcactaatcaataactcacatctgttccctcggtcacgcattctgcacccgttccacccctcaaaaccacacacatacccttcaaaactgcacgtcccaccattgtaactcacgtccccttcccccttctttatttgagagaaatattcgcaagtagccaatcgggtcagcttacattgtgccgaccccagcccatgggggaaggacatggaggcagggattgcgttaaggatataaaacccccctggcctcctttgttctctgtgctcttggatcttgattgacgtgggtggcacccttctgcagaagtaaactgccttgctgagagaacttttgcctcagtgctggtttcactttgcagcacccagcatttattgctagagcatttttatattcaacatgacctaaggtgtaaagggattcacatggaagtttttgccattttcttcatcctttccattgtcgtcataggtcactcattcccaggtctagagaaagtttgatgcagtaactgttgtttgagatgagtggcttcacctgctcatctttctattgaatacatttataagtgatgtgcttcaagaaattcttcaaagctaaacataagacaaagtttctacatatcctagcaagataacatgaccatttaaaaactgatatgaagctacagaagagacctcagggatgagaggagaagaccacccccacaacttcagccagacctcagagaaacagactgtcatcacaccctagcataagaagagacactaagcagcgaggtttctgtcttcagcctgtagatagaaaaaatattcgaagaattatctcatgcctttaccaagtctcagctcaaattttgcagcttgagtattctgaaaaaaaaatttttttttgagacggagtctcgctctgtcgcccaggctggagtgcagtggcacaatctcggctcactgcaagctccgcttcccaggctcacgccattctcctgcctcagcctcccgagtagttgggactacaggcgcccaccaccacacccggctaattttcagtagagatggggtttcaccgtgttagccaggatggtctcgatctcctgacctcgtgatccacctgtctcagcctcccaaagtgctgggattacaggcgtgagccaccgcacctggccaaatatatttttaagtactggaaatttttataaactctgaattccaaggtaagatagctcagtttactttataaaggtgtacctgcatgcatgtatacataagccattgattttccaccattgtgggcaagagggtataaggtgagacttaacattaaatgggattaaaggaaatgattaggatactgggactcttaagtttgtaaatcactgcctcagatatttggttatatttaactaataggtacatgatatagtttggatgtttgtcccctccaaatctcacactgaaatgtaatccccagtgctggaggtggggcctggtgggaggtgttggatcatgggggcaaatcgctcatgaatggtttagtaatccctttgctgtaagtcagttcttgctctggtagttcacaccagatcaggttgtttaaaagagtatggaacgtctctactctgtctcttgctcctggtctcactatgtgatgtgcctgctcccgctttgctttctgctgtgattacaagcttcctgaggccctcaccagaagacaagcagatgttggtaccatgtttgtgcagcctgcaaaacagtgagccaattaaacctcttttcttgggccgggcttggtggctcacacctgtaatcccagcattttgggaggccaaggcgggcggatcacaaggtcaggagatcgagaccatcctggctagcacggtgaaaccctgtctctactaaaaatacaaaaaaattagccaggcgtggtggcggacacctgtagtcccagctgctcaggaggctgaagtgagagaatggtgtgaacccaggaggcggagcttgtagtgagccaagattgcgccactgcactgtctgggcaacaaagcaagactccacctcaaaaaaaaaaaaaaaaaaaaaaaagcctcttttctttataaattacccagcctcaggtattcctttatagcaacgcaagaacaggctaatacagaaaatttctgaaaatgtgaaagcagctttagaactgtgtaacaggcagaagttggaagagtttggagggctcagaaggaagaaggaatatgagagaaagtttggaacttcttggagactggttaaagggttgtgaccaaaatactggtagagatatggacagtgaaggtcaggctgaggaggtctcagatgaggaacttactgggaactggagcaaaggtcagccttgttattccctggcaaaggacctggcagcattgcgtctatgtcctaaggctttcgggaagtttgaacttaatagtaatgacttagagagtgtctggtgggagaaatttctaagcagcaaagtgttcaaggagaggcacggctgcttctaacagcttacagtcagatgtgggagcaaaggaattaaagttgaacattatatttaaaaagggaagcagagcataaaaacttggaaaattctcaacctggccatgcggtaaagaaggaaagagcattttcgggggaagaatttaagagggctgcagaaccaccacttgctgaagagactaacgtgactaaaagggagccaagtgctaagatccaagacaaaaggaaaaaggctttgaaggcatttcggggatcttccaggccacctctcccatcacaaacccagaagcctaggagaaaagaatgatttagcgggccagttccagggccccgctgccctgtgcagcctttggacaccattctccacgttccagccactcaaggctccagccttggctcaaaggggcccaggtatagcttgggttgcagctctgcaggttgcaagctgtaagctttggtggttccaggtagtattaagcctgcagatgctcagattgcaagcatgaaggaggcttagtggcttccacctagatttcaaaggatgtatggaaaagccacggtgcccaggaaggagactgacattggggcatagctcccacagaggaactctattaggattggattcccccgtacccccacaagaatccctaccagggcactgcctagtggagctggagggccacctccctccagaccccagaatggtagattcactggcagcttgtgccctctgcctgaaaaacctgcaggcactcaactccaacccataagagcagccatgggggttgcaccctgcaaagccacagggctggagctgcccaaagccttgagatcccatatcttgcactagtgtgtcttgacgcaggacatggagtcaaaggagattttggagctttaagatttaatgactgctcttctgggtttacaacttgcatgaggtctgttgcctttttcttttggcctgtgtctcccttttgaaatgagaatgtttaaccagtgcccataccaccatcttctcttggaagtaaataacatgtctttgatttcacaggctcacggggaaggaattcatcttcagatgagacttttgacttgggacttgggacttttgattgagttgatggtggaacaacttatgactttggggaggtatttagaagggatgattgtattttgcaatttgagaaggtattgagatttaaggggcaggggcagaataatatagtttagatgtttgtctcctccaaatctcttgttgaaatgtaatccccaatattggagatggggactggtgggacgtattcggatcatgggggcaaatccctcatgaatggcttagtgtcatccccatggtgctgtcagttctcactctagtagttcatgcgagatctggttgtttaaaagagcatgcaatctcccctgcctgctcgctcccattctcatcatgtgatgttcctgctcctgcttcactttttgccatgattgtgagcttcctgaggccctcgccagaagctgagcagaggctggtgccatgcttgtagagcctgcagaaccgtgagcccgttaaacctcttttctttataaattacccagtctcaggtattcctttttagtaatgcaaaaacagactaacacagtacatctctccctaaaagtgcttggtatttgcctacatttatgaagaacatagcattaaatacagcaccaaataggtgctcaatattttcacggaaagaattaatgcacatgtgtaagaatcaagcagcacagtcacctaatgaggaatagccaaatttttaaacaaagtttttctttttgagacaaaatttcactcttgttgcccaggctggagtgcaatggcatgatgtcggctcactgcaacctccgcctcctgggttcaagcaattctcctgcctcagcctccagagtagctgggattacaggctcccgccaccacacccaactaatttttgtatttttagtagagatggagcttcaccatgttggccaggctggtctcgagcccctgacctcaggtgatccacccgcctcggcctcccaaagtgctgggattacaggtgtgagccactgcgcccggcttaaacaaactttttaatggagaaaatgtcatccaataaatcaaccaagtgtgacccatcctcgtgtgagccatcctagaacatcacccactgtggaccaggttgccagtgttataaccatgtgctccccaagtgaatcagtaagagaagctatgtggggaagagcaatgtatgatcttactgtgctaggtttgaaggcatcaggcacagcccttctaaagtgtgtgtatttctcagacctagctcttctcattctgatctttttgaaccatttcttgttttgtccccagacctgctagaggcaaagcatcttctgggtcagctcattaagataggcaaggacaagaagagtcttgagtgggacctagacagtggcacctgttgactgaaaaggcagtacaccaaaagatggagacaccctgcagggtgttgatgatggtgaagagataggcaagagcatagcaactgggcccacctgcagaatgcccaaacaccaggtgcagcccaagatgaacagctgatctgtcaccttgaatgtcagcatcctggattggatgagaaaagaggcccataatatacccataataacaaaggctatttcatctccatcccatggatatctctgccctctcctgttggtcatttctcacacttgagttttgatcaacctagatctttactgttgataattccccacaaaagaaatgccatttggatccaggaaagcattttaacttggaccaaggagagtagtgacacttcataatatgggttgcaaaagaagtgtgatgatcaacacaaaaaaacagttgcatttctatgcactaacaatgaacaacccaaaaaggaaattgagaaaacaaatccatttacaacagcattaaaaacaataaaatactttggaattaacttaaccaatgagatgaagaacctgtacactgaaagctaaaaagcattgctgaaagaacttaaaagagacacaaatagacatactatgttcacgggctgaatgacttaatatttttgcagaaacagaaaaatatccatcctaaaactcatatgaaatctcaagaataccaaaccaagaagtcaaaaccatcttgaaaaagaacagagttggaggtctcacaccacacttatttcaaaatttattgtaaaactacagtaatcaacacaatacggtaatggcataaagataagacatatagaccaatggaatcgaggagggagcccagaaataaaacctcacatttatagtcaaatgaattttgacaagggtgccaagaccattcaatggggaaaggacagtcttcaacaaatggtgctgggaaaactggatatgcacatgcaaaagaatgaagttggactcttttcttactccatatataaaaattaacttggctgggcatggtggctcacacctgtaatcccagcactttgggaggctaaggcaggcagatcacaaggtcaggagatcaagaccatcctggctaacatggtgaaaccccatctctactaaaaatacaaaaaaattagccaggcatggtggtatgcgcctgtagtcccagctactcaggaggctgaggcaggagaatcacttgaaccggggaggcggaggttgcggtaagccaagattgcgccattgcactccagcctgggcaacacagcaagactccgtctcaaaaacacaaaaacgaacaaacaaacaaacagaaattaactcataatacatcaaagacctaaatgcaagagctgaaacaataagactcttagaagaaaacatagggggtaagcctcacgacattagatttgcaatgattttttggatatgacatctaaagaaaacaaaaaatatctataagttggactacaccaaaatcttagacttctgcatatcaaaggatacaattaataaagtaaaaaggtaacccctagaatgggagaaaatatttgcaaatcatatatctgataaggggttaatattcagaatatataaagaacttccacaactcaacaacaacaaaaaactaatttaaaaatggtcaaaggacttagacatttttccacagatgacatacaaatggtcacaactaagaaggaaaagcccaacgtgtgttcttccagcctctgcttctgtcatgtttgtcaacactccattggccaaagaaagtcacatgaccaagctcgtcttcaaggtttggagaaataaactccacctcttgataggaggagctgcaaagtcacatggaaaagacatatacatagagggatagaagaagtcattgtggcaaccatctaccactgcatcattactt